(19) United States
(12) Patent Application Publication
(10) Pub. No.: US 2013/0012493 A1
(43) Pub. Date: Jan. 10, 2013

(54) NOVEL SPIRO IMIDAZOLONE DERIVATIVES AS GLUCAGON RECEPTOR ANTAGONISTS, COMPOSITIONS, AND METHODS FOR THEIR USE

(76) Inventors: He Zhao, Madison, CT (US); Ping Chen, Slingerlands, NY (US); Peishan Lin, Schenectady, NY (US); Peng Dai, Guilderland, NY (US); Jason Louis Davis, Albany, NY (US); Duane E. DeMong, Somerset, NJ (US); Michael W. Miller, Scotch Plains, NJ (US); Xing Dai, Cranford, NJ (US); Michael K. Wong, Somerset, NJ (US); Brian J. Lavey, New Providence, NJ (US); Wensheng Yu, Edison, NJ (US); Guowei Zhou, Somerset, NJ (US); Andrew W. Stamford, Chatham Township, NJ (US); Joseph A. Kozlowski, Princeton, NJ (US); William J. Greenlee, Teaneck, NJ (US)

(21) Appl. No.: 13/636,276

(22) PCT Filed: Mar. 22, 2011
(86) PCT No.: PCT/US11/29333

§ 371 Date: Sep. 20, 2012

Related U.S. Application Data
(60) Provisional application No. 61/317,771, filed on Mar. 26, 2010.

(51) Int. Cl. A61K 31/454; A61P 3/10; C07D 403/12; A61K 31/4178; A61K 31/5386; A61K 31/55; A61K 31/496; C07D 413/14; A61K 31/5377; C07D 498/08; C07D 401/14; C07D 403/14

(52) U.S. Cl. 514/210.21; 546/210; 514/322; 548/253; 514/381; 540/603; 514/217.09; 544/370; 514/254.06; 544/139; 514/234.5; 544/105; 514/230.5

(57) ABSTRACT

The present invention relates to compounds of the general formula (I): wherein ring A, ring B, G, R³, Z, L¹, and L² are selected independently of each other and are as defined herein, to compositions comprising the compounds, and to methods of using the compounds as glucagon receptor antagonists and for the treatment or prevention of type 2 diabetes and conditions related thereto.

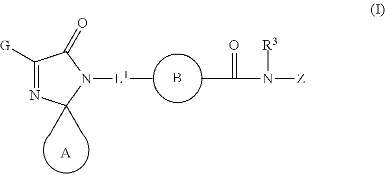

### FIELD OF THE INVENTION

The present invention relates to certain novel compounds as glucagon receptor antagonists, compositions comprising these compounds, and methods for their use in treating, preventing, or delaying the onset of type 2 diabetes and related conditions.

### BACKGROUND OF THE INVENTION

Diabetes refers to a disease state or process derived from multiple causative factors and is characterized by elevated levels of plasma glucose (hyperglycemia) in the fasting state or after administration of glucose during a glucose tolerance test. Persistent or uncontrolled hyperglycemia is associated with a wide range of pathologies. Diabetes mellitus, is associated with elevated fasting blood glucose levels and increased and premature cardiovascular disease and premature mortality. It is also related directly and indirectly to various metabolic conditions, including alterations of lipid, lipoprotein, apolipoprotein metabolism and other metabolic and hemodynamic diseases. As such, the diabetic patient is at increased risk of macrovascular and microvascular complications. Such complications can lead to diseases and conditions such as coronary heart disease, stroke, peripheral vascular disease, hypertension, nephropathy, neuropathy, and retinopathy. Accordingly, therapeutic control and correction of glucose homeostasis is regarded as important in the clinical management and treatment of diabetes mellitus.

There are two generally recognized forms of diabetes. In type 1 diabetes, or insulin-dependent diabetes mellitus (IDDM), the diabetic patient's pancreas is incapable of producing adequate amounts of insulin, the hormone which regulates glucose uptake and utilization by cells. In type 2 diabetes, or noninsulin dependent diabetes mellitus (NIDDM), patients often produce plasma insulin levels comparable to those of nondiabetic subjects; however, the cells of patients suffering from type 2 diabetes develop a resistance to the effect of insulin, even in normal or elevated plasma levels, on glucose and lipid metabolism, especially in the main insulin-sensitive tissues (muscle, liver and adipose tissue).

Insulin resistance is not associated with a diminished number of cellular insulin receptors but rather with a post-insulin receptor binding defect that is not well understood. This cellular resistance to insulin results in insufficient insulin activation of cellular glucose uptake, oxidation, and storage in muscle, and inadequate insulin repression of lipolysis in adipose tissue, and of glucose production and secretion in the liver. A net effect of decreased sensitivity to insulin is high levels of insulin circulating in the blood without appropriate reduction in plasma glucose (hyperglycemia). Hyperinsulinemia is a risk factor for developing hypertension and may also contribute to vascular disease.

The available treatments for type 2 diabetes, some of which have not changed substantially in many years, are used alone and in combination. Many of these treatments have recognized limitations, however. For example, while physical exercise and reductions in dietary intake of fat, high glycemic carbohydrates, and calories can dramatically improve the diabetic condition, compliance with this treatment is very poor because of well-entrenched sedentary lifestyles and excess food consumption, especially of foods containing high amounts of saturated fat. Increasing the plasma level of insulin by administration of sulfonylureas (e.g. tolbutamide and glipizide) or meglitinide, which stimulate the pancreatic beta-cells to secrete more insulin, and/or by injection of insulin when sulfonylureas or meglitinide become ineffective, can result in insulin concentrations high enough to stimulate insulin-resistance in tissues. However, dangerously low levels of plasma glucose can result from administration of insulin or insulin secretagogues (sulfonylureas or meglitinide), and an increased level of insulin resistance due to the even higher plasma insulin levels can occur. The biguanides are a separate class of agents that can increase insulin sensitivity and bring about some degree of correction of hyperglycemia. These agents, however, can induce lactic acidosis, nausea and diarrhea.

The glitazones (i.e. 5-benzylthiazolidine-2,4-diones) are another class of compounds that have proven useful for the treatment of type 2 diabetes. These agents increase insulin sensitivity in muscle, liver and adipose tissue in several animal models of type 2 diabetes, resulting in partial or complete correction of the elevated plasma levels of glucose without occurrence of hypoglycemia. The glitazones that are currently marketed are agonists of the peroxisome proliferator activated receptor (PPAR), primarily the PPAR-gamma subtype. PPAR-gamma agonism is generally believed to be responsible for the improved insulin sensititization that is observed with the glitazones. Newer PPAR agonists that are being tested for treatment of Type II diabetes are agonists of the alpha, gamma or delta subtype, or a combination thereof, and in many cases are chemically different from the glitazones (i.e., they are not thiazolidinediones). Serious side effects (e.g. liver toxicity) have been noted in some patients treated with glitazone drugs, such as troglitazone.

Compounds that are inhibitors of the dipeptidyl peptidase-IV (DPP-IV) enzyme are also under investigation as drugs that may be useful in the treatment of diabetes, and particularly type 2 diabetes.

Additional methods of treating hyperglycemia and diabetes are currently under investigation. New biochemical approaches include treatment with alpha-glucosidase inhibitors (e.g. acarbose) and protein tyrosine phosphatase-1B (PTP-1B) inhibitors.

Other approaches to treating hyperglycemia, diabetes, and indications attendant thereto have focused on the glucagon hormone receptor. Glucagon and insulin are the two primary hormones regulating plasma glucose levels. Insulin, released in response to a meal, increases the uptake of glucose into insulin-sensitive tissues such as skeletal muscle and fat. Glucagon, which is secreted by alpha cells in pancreatic islets in response to decreased postprandial glucose levels or during fasting, signals the production and release of glucose from the liver. Glucagon binds to specific receptors in liver cells that trigger glycogenolysis and an increase in gluconeogenesis through cAMP-mediated events. These responses generate increases in plasma glucose levels (e.g., hepatic glucose production), which help to regulate glucose homeostasis.

Type 2 diabetic patients typically have fasting hyperglycemia that is associated with elevated rates of hepatic glucose production. This is due to increased gluconeogenesis coupled with hepatic insulin resistance. Such patients typically have a relative deficiency in their fasting and postprandial insulin-to-glucagon ratio that contributes to their hyperglycemic state. Several studies have demonstrated that hepatic glucose production correlates with fasting plasma glucose levels, suggesting that chronic hepatic glucagon receptor antagonism should improve this condition. In addition, defects in rapid postprandial insulin secretion, as well as ineffective suppression of glucagon secretion, lead to increased glucagon levels that elevate hepatic glucose production and contribute to hyperglycemia. Suppression of elevated postprandial glucagon levels in type 2 diabetics with somatostatin has been shown to lower blood glucose concentrations. This indicates that acute postprandial glucagon receptor antagonism would also be beneficial. Based on these and other data, glucagon receptor antagonism holds promise as a potential treatment of type 2 diabetes by reducing hyperglycemia. There is thus a need in the art for small-molecule glucagon receptor antagonists with good safety profiles and efficacy that are useful for the treatment of hyperglycemia, diabetes, and related metabolic diseases and indications. The present invention addresses that need.

### SUMMARY OF THE INVENTION

In one embodiment, the compounds of the invention have the general structure shown in Formula (A):

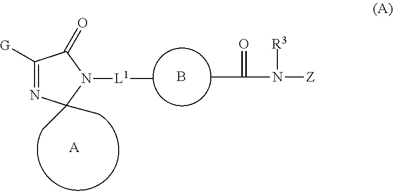

and include pharmaceutically acceptable salts, solvates, esters, prodrugs, tautomers, and isomers of said compounds, wherein ring A, ring B, L¹, G, R³, and Z are selected independently of each other and are as defined below.

The invention also relates to compositions, including pharmaceutically acceptable compositions, comprising the compounds of the invention (alone and in combination with one or more additional therapeutic agents), and to methods of using such compounds and compositions as glucagon receptor antagonists and for the treatment or prevention of type 2 diabetes and conditions related thereto.

### DETAILED DESCRIPTION OF THE INVENTION

In one embodiment, the compounds of the invention have the general structure shown in Formula (A):

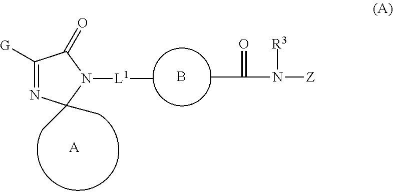

and include pharmaceutically acceptable salts, solvates, esters, prodrugs, tautomers, and isomers of said compounds,

wherein ring A, ring B, L¹, G, R³, and Z are selected independently of each other and wherein:

L¹ is selected from the group consisting of a bond, —N(R⁴)—, —N(R⁴)—(C(R^{5A})₂)—(C(R⁵)₂)_{q}—, —(C(R^{5A})₂)—(C(R⁵)₂)ᵣ—(C(R^{5A})₂)—N(R⁴)—, —O—, —O—(C(R^{5A})₂)—(C(R⁵)₂)_{q}—, —(C(R^{5A})₂)—(C(R⁵)₂)ᵣ—(C(R^{5A})₂)—O—, and —(C(R^{5A})₂)—(C(R⁵)₂)ₛ—,

each q is independently an integer from 0 to 5;

each r is independently an integer from 0 to 3;

s is an integer from 0 to 5;

ring A represents a spirocycloalkyl ring or a spirocycloalkenyl ring, wherein said ring A is substituted on one or more available ring carbon atoms with from 0 to 5 independently selected R² groups,

or, alternatively, ring A represents a spiroheterocycloalkyl ring or a spiroheterocycloalkenyl ring, wherein said ring A is substituted on one or more available ring carbon atoms with from 0 to 5 independently selected R² groups, and wherein said ring A is optionally further substituted on one or more available ring nitrogen atoms (when present) with from 0 to 3 R^{2A} groups;

ring B is a phenyl ring, wherein said phenyl ring is (in addition to the -L¹- and —C(O)N(R³)—Z moieties shown) optionally further substituted with one or more substituents R^{a}, wherein each R^{a} (when present) is independently selected from the group consisting of halo, —OH, —SF₅, —OSF₅, alkyl, haloalkyl, heteroalkyl, hydroxyalkyl, alkoxy, and —O-haloalkyl,

or ring B is a 5-membered heteroaromatic ring containing from 1 to 3 ring heteroatoms independently selected from N, O, and S, wherein said 5-membered heteroaromatic ring is (in addition to the and —C(O)N(R³)—Z moieties shown) optionally further substituted with one or more substituents R^{a}, wherein each R^{a} (when present) is independently selected from the group consisting of halo, —OH, —SF₅, —OSF₅, alkyl, haloalkyl, heteroalkyl, hydroxyalkyl, alkoxy, and —O-haloalkyl,

or ring B is a 6-membered heteroaromatic ring containing from 1 to 3 ring nitrogen atoms, wherein said 6-membered heteroaromatic ring is (in addition to -L- and —C(O)N(R³)Z moieties shown) optionally further substituted with one or more substituents R^{a}, wherein each R^{a} (when present) is independently selected from the group consisting of halo, —OH, —SF₅, —OSF₅, alkyl, haloalkyl, hydroxyalkyl, alkoxy, and —O-haloalkyl;

G is independently selected from the group consisting of:

(1) hydrogen, —NH₂, —OH, halo, —SH, —SO₂H, CO₂H, —SF₅, —OSF₅, cyano, —NO₂, —CHO,

(2) cycloalkyl, —O-cycloalkyl, —C(O)-cycloalkyl, —CO₂-cycloalkyl, —S-cycloalkyl, —S(O)-cycloalkyl, —S(O)₂-cycloalkyl, —N(R¹)-cycloalkyl, —C(O)—N(R¹)-cycloalkyl, —N(R¹)—C(O)-cycloalkyl, —N(R¹)—C(O)—N(R¹)-cycloalkyl, —N(R¹)—S(O)-cycloalkyl, —N(R¹)—S(O)₂-cycloalkyl, —N(R¹)—S(O)₂—N(R¹)-cycloalkyl, —S(O)—N(R¹)-cycloalkyl, —S(O)₂—N(R¹)-cycloalkyl,

(3) heterocycloalkyl, —O-heterocycloalkyl, —C(O)-heterocycloalkyl, —CO₂-heterocycloalkyl, —S-heterocycloalkyl, —S(O)-heterocycloalkyl, —S(O)₂-heterocycloalkyl, —N(R¹)-heterocycloalkyl, —C(O)—N(R¹)-heterocycloalkyl, —N(R¹)—C(O)-heterocycloalkyl, —N(R¹)—C(O)—N(R¹)-heterocycloalkyl, —N(R¹)—S(O)-heterocycloalkyl, —N(R¹)—S(O)₂-heterocycloalkyl, —N(R¹)—S(O)₂—N(R¹)-heterocycloalkyl, —S(O)—N(R¹)-heterocycloalkyl, —S(O)₂—N(R¹)-heterocycloalkyl,

(4) cycloalkenyl, —O-cycloalkenyl, —C(O)-cycloalkenyl, —CO₂-cycloalkenyl, —S-cycloalkenyl, —S(O)-cycloalkenyl, —S(O)₂-cycloalkenyl, —N(R¹)-cycloalkenyl, —C(O)—N(R¹)-cycloalkenyl, —N(R¹)—C(O)-cycloalkenyl, —N(R¹)—C(O)—N(R¹)-cycloalkenyl, —N(R¹)—S(O)-cycloalkenyl, —N(R¹)—S(O)₂-cycloalkenyl, —N(R¹)—S(O)₂—N(R¹)-cycloalkenyl, —S(O)—N(R¹)-cycloalkenyi, —S(O)₂—N(R¹)-cycloalkenyl,

(5) heterocycloalkenyl, —O-heterocycloalkenyl, —C(O)-heterocycloalkenyl, —CO₂-heterocycloalkenyl, —S-heterocycloalkenyl, —S(O)-heterocycloalkenyl, —S(O)₂-heterocycloalkenyl, —N(R¹)-heterocycloalkenyl, —C(O)—N(R¹)-heterocycloalkenyl, and —N(R¹)—C(O)-heterocycloalkenyl, —N(R¹)—C(O)—N(R¹)-heterocycloalkenyl, —N(R¹)—S(O)-heterocycloalkenyl, —N(R¹)—S(O)₂-heterocycloalkenyl, —N(R¹)—S(O)₂—N(R¹)-heterocycloalkenyl, —S(O)—N(R¹)-heterocycloalkenyl, —S(O)₂—N(R¹)-heterocycloalkenyl,

(6) alkyl, —O-alkyl, —C(O)-alkyl, —CO₂-alkyl, —S-alkyl, —S(O)-alkyl, —S(O)₂-alkyl, —N(R¹)-alkyl, —C(O)—N(R¹)-alkyl, —N(R¹)—C(O)-alkyl, —N(R¹)—C(O)—N(R¹)-alkyl, —N(R¹)—S(O)-alkyl, —N(R¹)—S(O)₂-alkyl, —N(R¹)—S(O)₂—N(R¹)-alkyl, —S(O)—N(R¹)-alkyl, —S(O)₂—N(R¹)-alkyl,

(7) heteroalkyl, —O-heteroalkyl, —C(O)-heteroalkyl, —CO₂-heteroalkyl, —S-heteroalkyl, —S(O)-heteroalkyl, —S(O)₂-heteroalkyl, —N(R¹)-heteroalkyl, —C(O)—N(R¹)-heteroalkyl, —N(R¹)—C(O)-heteroalkyl, —N(R¹)—C(O)—N(R¹)-heteroalkyl, —N(R¹)—S(O)-heteroalkyl, —N(R¹)—S(O)₂-heteroalkyl, —N(R¹)—S(O)₂—N(R¹)-heteroalkyl, —S(O)—N(R¹)-heteroalkyl, —S(O)₂—N(R¹)-heteroalkyl,

(8) alkenyl, —O-alkenyl, —C(O)-alkenyl, —CO₂-alkenyl, —S-alkenyl, —S(O)-alkenyl, —S(O)₂-alkenyl, —N(R¹)-alkenyl, —C(O)—N(R¹)-alkenyl, —N(R¹)—C(O)-alkenyl, —N(R¹)—C(O)—N(R¹)-alkenyl, —N(R¹)—S(O)-alkenyl, —N(R¹)—S(O)₂-alkenyl, —N(R¹)—S(O)₂—N(R¹)-alkenyl, —S(O)—N(R¹)-alkenyl, —S(O)₂—N(R¹)-alkenyl,

(10) alkynyl, —O-alkynyl, —C(O)-alkynyl, —CO₂-alkynyl, —S-alkynyl, —S(O)-alkynyl, —S(O)₂-alkynyl, —N(R¹)-alkynyl, —C(O)—N(R¹)-alkynyl, —N(R¹)—C(O)-alkynyl, —N(R¹)—C(O)—N(R¹)-alkynyl, —N(R¹)—S(O)-alkynyl, —N(R¹)—S(O)₂-alkynyl, —N(R¹)—S(O)₂—N(R¹)-alkynyl, —S(O)—N(R¹)-alkynyl, and —S(O)₂—N(R¹)-alkynyl;

wherein said heteroalkyl, said heterocycloalkyl, and said heterocycloalkenyl of G may be connected through any available carbon or heteroatom,

and wherein said cycloalkyl, said heterocycloalkyl, said alkenyl, said alkynyl, said cycloalkenyl, and said heterocycloalkenyl of G (when present) are unsubstituted or substituted with one or more groups independently selected from:

(1a) —NH₂, —OH, halo, —SH, —SO₂H, CO₂H, —Si(R⁷)₃, —SF₅, —OSF₅, cyano, —NO₂, —CHO,

(2a) cycloalkyl, —O-cycloalkyl, —C(O)-cycloalkyl, —CO₂-cycloalkyl, —S-cycloalkyl, —S(O)-cycloalkyl, —S(O)₂-cycloalkyl, —N(R²⁰)-cycloalkyl, —C(O)—N(R²⁰)-cycloalkyl, —N(R²⁰)—C(O)-cycloalkyl, —N(R²⁰)—C(O)—N(R²⁰)-cycloalkyl, —N(R²⁰)—S(O)-cycloalkyl, —N(R²⁰)—S(O)₂-cycloalkyl, —N(R²⁰)—S(O)₂—N(R²⁰)-cycloalkyl, —S(O)—N(R²⁰)-cycloalkyl, —S(O)₂—N(R²⁰)-cycloalkyl,

(3a) heterocycloalkyl, —O-heterocycloalkyl, —C(O)-heterocycloalkyl, —CO₂-heterocycloalkyl, —S-heterocycloalkyl, —S(O)-heterocycloalkyl, —S(O)₂-heterocycloalkyl, —N(R²⁰)-heterocycloalkyl, —C(O)—N(R²⁰)-heterocycloalkyl, —N(R²⁰)—C(O)-heterocycloalkyl, —N(R²⁰)—C(O)—N(R²⁰)-heterocycloalkyl, —N(R²⁰)—S(O)-heterocycloalkyl, —N(R²⁰)—S(O)₂-heterocycloalkyl, —N(R²⁰)—S(O)₂—N(R²⁰)-heterocycloalkyl, —S(O)—N(R²⁰)-heterocycloalkyl, —S(O)₂—N(R²⁰)-heterocycloalkyl,

(4a) cycloalkenyl, —O-cycloalkenyl, —C(O)-cycloalkenyl, —CO₂-cycloalkenyl, —S-cycloalkenyl, —S(O)-cycloalkenyl, —S(O)₂-cycloalkenyl, —N(R²⁰)-cycloalkenyl, —C(O)—N(R²⁰)-cycloalkenyl, —N(R²⁰)—C(O)-cycloalkenyl, —N(R²⁰)—C(O)—N(R²⁰)-cycloalkenyl, —N(R²⁰)—S(O)-cycloalkenyl, —N(R²⁰)—S(O)₂-cycloalkenyl, —N(R²⁰)—S(O)₂—N(R²⁰)-cycloalkenyl, —S(O)—N(R²⁰)-cycloalkenyl, —S(O)₂—N(R²⁰)-cycloalkenyl,

(5a) heterocycloalkenyl, —O-heterocycloalkenyl, —C(O)-heterocycloalkenyl, —CO₂-heterocycloalkenyl, —S-heterocycloalkenyl, —S(O)-heterocycloalkenyl, —S(O)₂-heterocycloalkenyl, —N(R²⁰)-heterocycloalkenyl, —C(O)—N(R²⁰)-heterocycloalkenyl, and —N(R²⁰)—C(O)-heterocycloalkenyl, —N(R²⁰)—C(O)—N(R²⁰)-heterocycloalkenyl, —N(R²⁰)—S(O)-heterocycloalkenyl, —N(R²⁰)—S(O)₂-heterocycloalkenyl, —N(R²⁰)—S(O)₂—N(R²⁰)-heterocycloalkenyl, —S(O)—N(R²⁰)-heterocycloalkenyl, —S(O)₂—N(R²⁰)-heterocycloalkenyl,

(6a) alkyl, —O-alkyl, —C(O)-alkyl, —CO₂-alkyl, —S-alkyl, —S(O)-alkyl, —S(O)₂-alkyl, —N(R²⁰)-alkyl, —C(O)—N(R²⁰)-alkyl, —N(R²⁰)—C(O)-alkyl, —N(R²⁰)—C(O)—N(R²⁰)-alkyl, —N(R²⁰)—S(O)-alkyl, —N(R²⁰)—S(O)₂-alkyl, —N(R²⁰)—S(O)₂—N(R²⁰)-alkyl, —S(O)—N(R²⁰)-alkyl, —S(O)₂—N(R²⁰)-alkyl,

(7a) heteroalkyl, —O-heteroalkyl, —C(O)-heteroalkyl, —CO₂-heteroalkyl, —S-heteroalkyl, —S(O)-heteroalkyl, —S(O)₂-heteroalkyl, —N(R²⁰)-heteroalkyl, —C(O)—N(R²⁰)-heteroalkyl, —N(R²⁰)—C(O)-heteroalkyl, —N(R²⁰)—C(O)—N(R²⁰)-heteroalkyl, —N(R²⁰)—S(O)-heteroalkyl, —N(R²⁰)—S(O)₂-heteroalkyl, —N(R²⁰)—S(O)₂—N(R²⁰)-heteroalkyl, —S(O)—N(R²⁰)-heteroalkyl, —S(O)₂—N(R²⁰)-heteroalkyl,

(8a) alkenyl, —O-alkenyl, —C(O)-alkenyl, —CO₂-alkenyl, —S-alkenyl, —S(O)-alkenyl, —S(O)₂-alkenyl, —N(R²⁰)-alkenyl, —C(O)—N(R²⁰)-alkenyl, —N(R²⁰)—C(O)-alkenyl, N(R²⁰)—C(O)—N(R²⁰)-alkenyl, —N(R²⁰)—S(O)-alkenyl, —N(R²⁰)—S(O)₂-alkenyl, —N(R²⁰)—S(O)₂—N(R²⁰)-alkenyl, —S(O)—N(R²⁰)-alkenyl, —S(O)₂—N(R²⁰)-alkenyl,

(10a) alkynyl, —O-alkynyl, —C(O)-alkynyl, —CO₂-alkynyl, —S-alkynyl, —S(O)-alkynyl, —S(O)₂-alkynyl, —N(R²⁰)-alkynyl, —C(O)—N(R²⁰)-alkynyl, —N(R²⁰)—C(O)-alkynyl, —N(R²⁰)—C(O)—N(R²⁰)-alkynyl, —N(R²⁰)—S(O)-alkynyl, —N(R²⁰)—S(O)₂-alkynyl, —N(R²⁰)—S(O)₂—N(R²⁰)-alkynyl, —S(O)—N(R²⁰)-alkynyl, —S(O)₂—N(R²⁰)-alkynyl,

(12a) aryl, —O-aryl, —C(O)-aryl, —CO₂-aryl, —S-aryl, —S(O)-aryl, —S(O)₂-aryl, —N(R²⁰)-aryl, —C(O)—N(R²⁰)-aryl, —N(R²⁰)—C(O)-aryl, —N(R²⁰)—C(O)—N(R²⁰)-aryl, —N(R²⁰)—S(O)-aryl, —N(R²⁰)—S(O)₂-aryl, —N(R²⁰)—S(O)₂—N(R²⁰)-aryl, —S(O)—N(R²⁰)-aryl, —S(O)₂—N(R²⁰)-aryl,

(13a) heteroaryl, —O-heteroaryl, —C(O)-heteroaryl, —CO₂-heteroaryl, —S-heteroaryl, —S(O)-heteroaryl, —S(O)₂-heteroaryl, —N(R²⁰)-heteroaryl, —C(O)—N(R²⁰)-heteroaryl, —N(R²⁰)—C(O)-heteroaryl, —N(R²⁰)—C(O)—N(R²⁰)-heteroaryl, —N(R²⁰)—S(O)-heteroaryl, —N(R²⁰)—S(O)₂-heteroaryl, —N(R²⁰)—S(O)₂—N(R²⁰)-heteroaryl, —S(O)—N(R²⁰)-heteroaryl, —S(O)₂—N(R²⁰)-heteroaryl;
wherein each of said alkyl, said alkenyl, said alkynyl, said cycloalkyl, said cycloalkenyl, said aryl, said heteroalkyl, said heterocycloalkyl, said heterocycloalkenyl, and said heteroaryl of (1a) through (13a) (when present) are each optionally further substituted with one or more groups each independently selected from:
(i) —NH₂, —OH, halo, —SH, —SO₂H, CO₂H, —Si(R⁷)₃, —SF₅, —OSF₅, cyano, —NO₂, —CHO,
(ii) cycloalkyl, —O-cycloalkyl, —C(O)-cycloalkyl, —CO₂-cycloalkyl, —S-cycloalkyl, —S(O)-cycloalkyl, —S(O)₂-cycloalkyl, —N(R²⁰)-cycloalkyl, —C(O)—N(R²⁰)-cycloalkyl, —N(R²⁰)—C(O)-cycloalkyl, —N(R²⁰)—C(O)—N(R²⁰)-cycloalkyl, —N(R²⁰)—S(O)-cycloalkyl, —N(R²⁰)—S(O)₂-cycloalkyl, —N(R²⁰)—S(O)₂—N(R²⁰)-cycloalkyl, —S(O)—N(R²⁰)-cycloalkyl, —S(O)₂—N(R²⁰)-cycloalkyl,
(iii) heterocycloalkyl, —O-heterocycloalkyl, —C(O)-heterocycloalkyl, —CO₂-heterocycloalkyl, —S-heterocycloalkyl, —S(O)-heterocycloalkyl, —S(O)₂-heterocycloalkyl, —N(R²⁰)-heterocycloalkyl, —C(O)—N(R²⁰)-heterocycloalkyl, —N(R²⁰)—C(O)-heterocycloalkyl, —N(R²⁰)—C(O)—N(R²⁰)-heterocycloalkyl, —N(R²⁰)—S(O)-heterocycloalkyl, —N(R²⁰)—S(O)₂-heterocycloalkyl, —N(R²⁰)—S(O)₂—N(R²⁰)-heterocycloalkyl, —S(O)—N(R²⁰)-heterocycloalkyl, —S(O)₂—N(R²⁰)-heterocycloalkyl,
(iv) cycloalkenyl, —O-cycloalkenyl, —C(O)-cycloalkenyl, —CO₂-cycloalkenyl, —S-cycloalkenyl, —S(O)-cycloalkenyl, —S(O)₂-cycloalkenyl, —N(R²⁰)-cycloalkenyl, —C(O)—N(R²⁰)-cycloalkenyl, —N(R²⁰)—C(O)-cycloalkenyl, —N(R²⁰)—C(O)—N(R²⁰)-cycloalkenyl, —N(R²⁰)—S(O)-cycloalkenyl, —N(R²⁰)—S(O)₂-cycloalkenyl, —N(R²⁰)—S(O)₂—N(R²⁰)-cycloalkenyl, —S(O)—N(R²⁰)-cycloalkenyl, —S(O)₂—N(R²⁰)-cycloalkenyl,
(v) heterocycloalkenyl, —O-heterocycloalkenyl, —C(O)-heterocycloalkenyl, —CO₂-heterocycloalkenyl, —S-heterocycloalkenyl, —S(O)-heterocycloalkenyl, —S(O)₂-heterocycloalkenyl, —N(R²⁰)-heterocycloalkenyl, —C(O)—N(R²⁰)-heterocycloalkenyl, and —N(R²⁰)—C(O)-heterocycloalkenyl, —N(R²⁰)—C(O)—N(R²⁰)-heterocycloalkenyl, —N(R²⁰)—S(O)-heterocycloalkenyl, —N(R²⁰)—S(O)₂-heterocycloalkenyl, —N(R²⁰)—S(O)₂—N(R²⁰)-heterocycloalkenyl, —S(O)—N(R²⁰)-heterocycloalkenyl, —S(O)₂—N(R²⁰)-heterocycloalkenyl,
(vi) alkyl, —O-alkyl, —C(O)-alkyl, —CO₂-alkyl, —S-alkyl, —S(O)-alkyl, —S(O)₂-alkyl, —N(R²⁰)-alkyl, —C(O)—N(R²⁰)-alkyl, —N(R²⁰)—C(O)-alkyl, —N(R²⁰)—C(O)—N(R²⁰)-alkyl, —N(R²⁰)—S(O)-alkyl, —N(R²⁰)—S(O)₂-alkyl, —N(R²⁰)—S(O)₂—N(R²⁰)-alkyl, —S(O)—N(R²⁰)-alkyl, —S(O)₂—N(R²⁰)-alkyl,
(vii) heteroalkyl, —O-heteroalkyl, —C(O)-heteroalkyl, —CO₂-heteroalkyl, —S-heteroalkyl, —S(O)-heteroalkyl, —S(O)₂-heteroalkyl, —N(R²⁰)-heteroalkyl, —C(O)—N(R²⁰)-heteroalkyl, —N(R²⁰)—C(O)-heteroalkyl, —N(R²⁰)—C(O)—N(R²⁰)-heteroalkyl, —N(R²⁰)—S(O)-heteroalkyl, —N(R²⁰)—S(O)₂-heteroalkyl, —N(R²⁰)—S(O)₂—N(R²⁰)-heteroalkyl, —S(O)—N(R²⁰)-heteroalkyl, —S(O)₂—N(R²⁰)-heteroalkyl,
(viii) alkenyl, —O-alkenyl, —C(O)-alkenyl, —CO₂-alkenyl, —S-alkenyl, —S(O)-alkenyl, —S(O)₂-alkenyl, —N(R²⁰)-alkenyl, —C(O)—N(R²⁰)-alkenyl, —N(R²⁰)—C(O)-alkenyl, —N(R²⁰)—C(O)—N(R²⁰)-alkenyl, —N(R²⁰)—S(O)-alkenyl, —N(R²⁰)—S(O)₂-alkenyl, —N(R²⁰)—S(O)₂—N(R²⁰)-alkenyl, —S(O)—N(R²⁰)-alkenyl, —S(O)₂—N(R²⁰)-alkenyl,
(x) alkynyl, —O-alkynyl, —C(O)-alkynyl, —CO₂-alkynyl, —S-alkynyl, —S(O)-alkynyl, —S(O)₂-alkynyl, —N(R²⁰)-alkynyl, —C(O)—N(R²⁰)-alkynyl, —N(R²⁰)—C(O)-alkynyl, —N(R²⁰)—C(O)—N(R²⁰)-alkynyl, —N(R²⁰)—S(O)-alkynyl, N(R²⁰)—S(O)₂-alkynyl, —N(R²⁰)—S(O)₂—N(R²⁰)-alkynyl, —S(O)—N(R²⁰)-alkynyl, —S(O)₂—N(R²⁰)-alkynyl,
(xii) aryl, —O-aryl, —C(O)-aryl, —CO₂-aryl, —S-aryl, —S(O)-aryl, —S(O)₂-aryl, —N(R²⁰)-aryl, —C(O)—N(R²⁰)-aryl, —N(R²⁰)—C(O)-aryl, —N(R²⁰)—C(O)—N(R²⁰)-aryl, —N(R²⁰)—S(O)-aryl, —N(R²⁰)—S(O)₂-aryl, —N(R²⁰)—S(O)₂—N(R²⁰)-aryl, —S(O)—N(R²⁰)-aryl, —S(O)₂—N(R²⁰)-aryl,
(xiii) heteroaryl, —O-heteroaryl, —C(O)-heteroaryl, —CO₂-heteroaryl, —S-heteroaryl, —S(O)-heteroaryl, —S(O)₂-heteroaryl, —N(R²⁰)-heteroaryl, —C(O)—N(R²⁰)-heteroaryl, —N(R²⁰)—C(O)-heteroaryl, —N(R²⁰)—C(O)—N(R²⁰)-heteroaryl, —N(R²⁰)—S(O)-heteroaryl, —N(R²⁰)—S(O)₂-heteroaryl, —N(R²⁰)—S(O)₂—N(R²⁰)-heteroaryl, —S(O)—N(R²⁰)-heteroaryl, —S(O)₂—N(R²⁰)-heteroaryl;

and wherein said alkyl and said heteroalkyl of G (when present) are optionally further substituted with one or more groups independently selected from:

(1f) —NH₂, —OH, halo, —SH, —SO₂H, CO₂H, —Si(R⁷)₃, —SF₅, —OSF₅, cyano, —NO₂, —CHO,

(2f) cycloalkyl, —O-cycloalkyl, —C(O)-cycloalkyl, —CO₂-cycloalkyl, —S-cycloalkyl, —S(O)-cycloalkyl, —S(O)₂-cycloalkyl, —N(R²⁰)-cycloalkyl, —C(O)—N(R²⁰)-cycloalkyl, —N(R²⁰)—C(O)-cycloalkyl, —N(R²⁰)—C(O)—N(R²⁰)-cycloalkyl, —N(R²⁰)—S(O)-cycloalkyl, —N(R²⁰)—S(O)₂-cycloalkyl, —N(R²⁰)—S(O)₂—N(R²⁰)-cycloalkyl, —S(O)—N(R²⁰)-cycloalkyl, —S(O)₂—N(R²⁰)-cycloalkyl,

(3f) heterocycloalkyl, —O-heterocycloalkyl, —C(O)-heterocycloalkyl, —CO₂-heterocycloalkyl, —S-heterocycloalkyl, —S(O)-heterocycloalkyl, —S(O)₂-heterocycloalkyl, —N(R²⁰)-heterocycloalkyl, —C(O)—N(R²⁰)-heterocycloalkyl, —N(R²⁰)—C(O)-heterocycloalkyl, —N(R²⁰)—C(O)—N(R²⁰)-heterocycloalkyl, —N(R²⁰)—S(O)-heterocycloalkyl, —N(R²⁰)—S(O)₂-heterocycloalkyl, —N(R²⁰)—S(O)₂—N(R²⁰)-heterocycloalkyl, —S(O)—N(R²⁰)-heterocycloalkyl, —S(O)₂—N(R²⁰)-heterocycloalkyl,

(4f) cycloalkenyl, —O-cycloalkenyl, —C(O)-cycloalkenyl, —CO₂-cycloalkenyl, —S-cycloalkenyl, —S(O)-cycloalkenyl, —S(O)₂-cycloalkenyl, —N(R²⁰)-cycloalkenyl, —C(O)—N(R²⁰)-cycloalkenyl, —N(R²⁰)—C(O)-cycloalkenyl, —N(R²⁰)—C(O)—N(R²⁰)-cycloalkenyl, —N(R²⁰)—S(O)-cycloalkenyl, —N(R²⁰)—S(O)₂-cycloalkenyl, —N(R²⁰)—S(O)₂—N(R²⁰)-cycloalkenyl, —S(O)—N(R²⁰)-cycloalkenyl, —S(O)₂—N(R²⁰)-cycloalkenyl,

(5f) heterocycloalkenyl, —O-heterocycloalkenyl, —C(O)-heterocycloalkenyl, —CO₂-heterocycloalkenyl, —S-heterocycloalkenyl, —S(O)-heterocycloalkenyl, —S(O)₂-heterocycloalkenyl, —N(R²⁰)-heterocycloalkenyl, —C(O)—N(R²⁰)-heterocycloalkenyl, and)-N(R²⁰)—C(O)-heterocycloalkenyl, —N(R²⁰)—C(O)—N(R²⁰)-heterocycloalkenyl, —N(R²⁰)—S(O)-heterocycloalkenyl, —N(R²⁰)—S(O)₂-heterocycloalkenyl, —N(R²⁰)—S(O)₂—N(R²⁰)-heterocycloalkenyl, —S(O)—N(R²⁰)-heterocycloalkenyl, —S(O)₂—N(R²⁰)-heterocycloalkenyl,

(6f) alkyl, —O-alkyl, —C(O)-alkyl, —CO₂-alkyl, —S-alkyl, —S(O)-alkyl, —S(O)₂-alkyl, —N(R²⁰)-alkyl, —C(O)—N(R²⁰)-alkyl, —N(R²⁰)—C(O)-alkyl, —N(R²⁰)—C(O)—N(R²⁰)-alkyl, —N(R²⁰)—S(O)-alkyl, —N(R²⁰)—S(O)₂-alkyl, —N(R²⁰)—S(O)₂—N(R²⁰)-alkyl, —S(O)—N(R²⁰)-alkyl, —S(O)₂—N(R²⁰)-alkyl,

(7f) heteroalkyl, —O-heteroalkyl, —C(O)-heteroalkyl, —CO₂-heteroalkyl, —S-heteroalkyl, —S(O)-heteroalkyl, —S(O)₂-heteroalkyl, —N(R²⁰)-heteroalkyl, —C(O)—N(R²⁰)-heteroalkyl, —N(R²⁰)—C(O)-heteroalkyl, —N(R²⁰)—C(O)—N(R²⁰)-heteroalkyl, —N(R²⁰)—S(O)-heteroalkyl, —N(R²⁰)—S(O)₂-heteroalkyl, —N(R²⁰)—S(O)₂—N(R²⁰)-heteroalkyl, —S(O)—N(R²⁰)-heteroalkyl, —S(O)₂—N(R²⁰)-heteroalkyl,

(8f) alkenyl, —O-alkenyl, —C(O)-alkenyl, —CO₂-alkenyl, —S-alkenyl, —S(O)-alkenyl, —S(O)₂-alkenyl, —N(R²⁰)-alkenyl, —C(O)—N(R²⁰)-alkenyl, —N(R²⁰)—C(O)-alkenyl, —N(R²⁰)—C(O)—N(R²⁰)-alkenyl, —N(R²⁰)—S(O)-alkenyl, —N(R²⁰)—S(O)₂-alkenyl, —N(R²⁰)—S(O)₂—N(R²⁰)-alkenyl, —S(O)—N(R²⁰)-alkenyl, —S(O)₂—N(R²⁰)-alkenyl,

(10f) alkynyl, —O-alkynyl, —C(O)-alkynyl, —CO₂-alkynyl, —S-alkynyl, —S(O)-alkynyl, —S(O)₂-alkynyl, —N(R²⁰)-alkynyl, —C(O)—N(R²⁰)-alkynyl, —N(R²⁰)—C(O)-alkynyl, —N(R²⁰)—C(O)—N(R²⁰)-alkynyl, —N(R²⁰)—S(O)-alkynyl, —N(R²⁰)—S(O)₂-alkynyl, —N(R²⁰)—S(O)₂—N(R²⁰)-alkynyl, —S(O)—N(R²⁰)-alkynyl, —S(O)₂—N(R²⁰)-alkynyl;
wherein each of said alkyl, said alkenyl, said alkynyl, said cycloalkyl, said cycloalkenyl, said heteroalkyl, said heterocycloalkyl and said heterocycloalkenyl are unsubstituted or optionally independently substituted with one or more groups each independently selected from:
(i) —NH₂, —OH, halo, —SH, —SO₂H, CO₂H, —Si(R⁷)₃, —SF₅, —OSF₅, cyano, —NO₂, —CHO,
(ii) cycloalkyl, —O-cycloalkyl, —C(O)-cycloalkyl, —CO₂-cycloalkyl, —S-cycloalkyl, —S(O)-cycloalkyl, —S(O)₂-cycloalkyl, —N(R²⁰)-cycloalkyl, —C(O)—N(R²⁰)-cycloalkyl, —N(R²⁰)—C(O)-cycloalkyl, N(R²⁰)—C(O)—N(R²⁰)-cycloalkyl, —N(R²⁰)—S(O)-cycloalkyl, —N(R²⁰)—S(O)₂-cycloalkyl, —N(R²⁰)—S(O)₂—N(R²⁰)-cycloalkyl, —S(O)—N(R²⁰)-cycloalkyl, —S(O)₂—N(R²⁰)-cycloalkyl,
(iii) heterocycloalkyl, —O-heterocycloalkyl, —C(O)-heterocycloalkyl, —CO₂-heterocycloalkyl, —S-heterocycloalkyl, —S(O)-heterocycloalkyl, —S(O)₂-heterocycloalkyl, —N(R²⁰)-heterocycloalkyl, —C(O)—N(R²⁰)-heterocycloalkyl, —N(R²⁰)—C(O)-heterocycloalkyl, —N(R²⁰)—C(O)—N(R²⁰)-heterocycloalkyl, —N(R²⁰)—S(O)-heterocycloalkyl, —N(R²⁰)—S(O)₂-heterocycloalkyl, —N(R²⁰)—S(O)₂—N(R²⁰)-heterocycloalkyl, —S(O)—N(R²⁰)-heterocycloalkyl, —S(O)₂—N(R²⁰)-heterocycloalkyl,
(iv) cycloalkenyl, —O-cycloalkenyl, —C(O)-cycloalkenyl, —CO₂-cycloalkenyl, —S-cycloalkenyl, —S(O)-cycloalkenyl, —S(O)₂-cycloalkenyl, —N(R²⁰)-cycloalkenyl, —C(O)—N(R²⁰)-cycloalkenyl, —N(R²⁰)—C(O)-cycloalkenyl, —N(R²⁰)—C(O)—N(R²⁰)-cycloalkenyl, —N(R²⁰)—S(O)-cycloalkenyl, —N(R²⁰)—S(O)₂-cycloalkenyl, —N(R²⁰)—S(O)₂—N(R²⁰)-cycloalkenyl, —S(O)—N(R²⁰)-cycloalkenyl, —S(O)₂—N(R²⁰)-cycloalkenyl,
(v) heterocycloalkenyl, —O-heterocycloalkenyl, —C(O)-heterocycloalkenyl, —CO₂-heterocycloalkenyl, —S-heterocycloalkenyl, —S(O)-heterocycloalkenyl, —S(O)₂-heterocycloalkenyl, —N(R²⁰)-heterocycloalkenyl, —C(O)—N(R²⁰)-heterocycloalkenyl, and —N(R²⁰)—C(O)-heterocycloalkenyl, —N(R²⁰)—C(O)—N(R²⁰)-heterocycloalkenyl, —N(R²⁰)—S(O)-heterocycloalkenyl, —N(R²⁰)—S(O)₂-heterocycloalkenyl, —N(R²⁰)—S(O)₂—N(R²⁰)-heterocycloalkenyl, —S(O)—N(R²⁰)-heterocycloalkenyl, —S(O)₂—N(R²⁰)-heterocycloalkenyl,
(vi) alkyl, —O-alkyl, —C(O)-alkyl, —CO₂-alkyl, —S-alkyl, —S(O)-alkyl, —S(O)₂-alkyl, —N(R²⁰)-alkyl, —C(O)—N(R²⁰)-alkyl, —N(R²⁰)—C(O)-alkyl, —N(R²⁰)—C(O)—N(R²⁰)-alkyl, —N(R²⁰)—S(O)-alkyl, —N(R²⁰)—S(O)₂-alkyl, —N(R²⁰)—S(O)₂—N(R²⁰)-alkyl, —S(O)—N(R²⁰)-alkyl, —S(O)₂—N(R²⁰)-alkyl,
(vii) heteroalkyl, —O-heteroalkyl, —C(O)-heteroalkyl, —CO₂-heteroalkyl, —S-heteroalkyl, —S(O)-heteroalkyl, —S(O)₂-heteroalkyl, —N(R²⁰)-heteroalkyl, —C(O)—N(R²⁰)-heteroalkyl, —N(R²⁰)—C(O)-heteroalkyl, —N(R²⁰)—C(O)—N(R²⁰)-heteroalkyl, —N(R²⁰)—S(O)-heteroalkyl, —N(R²⁰)—S(O)₂-heteroalkyl, —N(R²⁰)—S(O)₂—N(R²⁰)-heteroalkyl, —S(O)—N(R²⁰)-heteroalkyl, —S(O)₂—N(R²⁰)-heteroalkyl,
(viii) alkenyl, —O-alkenyl, —C(O)-alkenyl, —CO₂-alkenyl, —S-alkenyl, —S(O)-alkenyl, —S(O)₂-alkenyl, —N(R²⁰)-alkenyl, —C(O)—N(R²⁰)-alkenyl, —N(R²⁰)—C(O)-alkenyl, —N(R²⁰)—C(O)—N(R²⁰)-alkenyl, —N(R²⁰)—S(O)-alkenyl, —N(R²⁰)—S(O)₂-alkenyl, —N(R²⁰)—S(O)₂—N(R²⁰)-alkenyl, —S(O)—N(R²⁰)-alkenyl, —S(O)₂—N(R²⁰)-alkenyl,
(x) alkynyl, —O-alkynyl, —C(O)-alkynyl, —CO₂-alkynyl, —S-alkynyl, —S(O)-alkynyl, —S(O)₂-alkynyl, —N(R²⁰)-alkynyl, —C(O)—N(R²⁰)-alkynyl, —N(R²⁰)—C(O)-alkynyl, —N(R²⁰)—C(O)—N(R²⁰)-alkynyl, —N(R²⁰)—S(O)-alkynyl, —N(R²⁰)—S(O)₂-alkynyl, —N(R²⁰)—S(O)₂—N(R²⁰)-alkynyl, —S(O)—N(R²⁰)-alkynyl, —S(O)₂—N(R²⁰)-alkynyl,

and wherein said cycloalkyl, said cycloalkenyl, said heterocycloalkyl, and heterocycloalkenyl (when present) of G are optionally unsubstituted or substituted with one or more groups independently selected from: spirocycloalkyl, spirocycloalkenyl, spiroheterocycloalkyl, and spiroheterocycloalkenyl, wherein said spirocycloalkyl, said spirocycloalkenyl, said spiroheterocycloalkyl, and said spiroheterocycloalkenyl are unsubstituted or substituted with one or more groups independently selected from (1a), (2a), (3a), (4a), (5a), (6a), (7a), (8a), (10a), (12a) and (13a) above;

each R¹ is independently selected from:

(1b) hydrogen,

(2b) cycloalkyl, —C(O)-cycloalkyl, —CO₂-cycloalkyl, —S(O)-cycloalkyl, —S(O)₂-cycloalkyl, —C(O)—N(R²⁰)-cycloalkyl, —S(O)—N(R²⁰)-cycloalkyl, —S(O)₂—N(R²⁰)-cycloalkyl,

(3b) heterocycloalkyl, —C(O)-heterocycloalkyl, —CO₂-heterocycloalkyl, —S(O)-heterocycloalkyl, —S(O)₂-heterocycloalkyl, —C(O)—N(R²⁰)-heterocycloalkyl, —S(O)—N(R²⁰)-heterocycloalkyl, —S(O)₂—N(R²⁰)-heterocycloalkyl,

(4b) cycloalkenyl, —C(O)-cycloalkenyl, —CO₂-cycloalkenyl, —S(O)-cycloalkenyl, —S(O)₂-cycloalkenyl, —C(O)—N(R²⁰)-cycloalkenyl, —S(O)—N(R²⁰)-cycloalkenyl, —S(O)₂—N(R²⁰)-cycloalkenyl,

(5b) heterocycloalkenyl, —C(O)-heterocycloalkenyl, —CO₂-heterocycloalkenyl, —S(O)-heterocycloalkenyl, —S(O)₂-heterocycloalkenyl, —C(O)—N(R²⁰)-heterocycloalkenyl, —S(O)—N(R²⁰)-heterocycloalkenyl, —S(O)₂—N(R²⁰)-heterocycloalkenyl,

(6b) alkyl, —C(O)-alkyl, —CO₂-alkyl, —S(O)-alkyl, —S(O)₂-alkyl, —C(O)—N(R²⁰)-alkyl, —S(O)—N(R²⁰)-alkyl, —S(O)₂—N(R²⁰)-alkyl,

(7b) heteroalkyl, —C(O)-heteroalkyl, —CO₂-heteroalkyl, —S(O)-heteroalkyl, —S(O)₂-heteroalkyl, —C(O)—N(R²⁰)-heteroalkyl, —S(O)—N(R²⁰)-heteroalkyl, —S(O)₂—N(R²⁰)-heteroalkyl,

(8b) alkenyl, —C(O)-alkenyl, —CO₂-alkenyl, —S(O)-alkenyl, —S(O)₂-alkenyl, —C(O)—N(R²⁰)-alkenyl, —S(O)—N(R²⁰)-alkenyl, —S(O)₂—N(R²⁰)-alkenyl,

(10b) alkynyl, —C(O)-alkynyl, —CO₂-alkynyl, —S(O)-alkynyl, —S(O)₂-alkynyl, —C(O)—N(R²⁰)-alkynyl, —S(O)—N(R²⁰)-alkynyl, —S(O)₂—N(R²⁰)-alkynyl;

wherein said heteroalkyl, said heterocycloalkyl, and said heterocycloalkenyl of R¹ may be connected through any available carbon or heteroatom,

and wherein said cycloalkyl said heterocycloalkyl, said alkenyl, said alkynyl, said cycloalkenyl, and said heterocycloalkenyl of R¹ are unsubstituted or substituted with one or more groups independently selected from (1a), (2a), (3a), (4a), (5a), (6a), (7a), (8a), (10a), (12a) and (13a) above:

and wherein said alkyl and said heteroalkyl of R¹ are unsubstituted or substituted with one or more groups independently selected from (10, (2f), (3f), (4f), (5f), (6f), (7f), (8f), and (100 above; each R² (when present) is independently selected from the group consisting of:

(1c) —NH₂, —OH, halo, —SH, —SO₂H, CO₂H, —SF₅, —OSF₅, cyano, —NO₂, —CHO,

(2c) cycloalkyl, —O-cycloalkyl, —C(O)-cycloalkyl, —CO₂-cycloalkyl, —S-cycloalkyl, —S(O)-cycloalkyl, —S(O)₂-cycloalkyl, —N(R²¹)-cycloalkyl, —C(O)—N(R²¹)-cycloalkyl, —N(R²¹)—C(O)-cycloalkyl, —N(R²¹)—C(O)—N(R²¹)-cycloalkyl, —N(R²¹)—S(O)-cycloalkyl, —N(R²¹)—S(O)₂-cycloalkyl, —N(R²¹)—S(O)₂—N(R²¹)-cycloalkyl, —S(O)—N(R²¹)-cycloalkyl, —S(O)₂—N(R²¹)-cycloalkyl,

(3c) heterocycloalkyl, —O-heterocycloalkyl, —C(O)-heterocycloalkyl, —CO₂-heterocycloalkyl, —S-heterocycloalkyl, —S(O)-heterocycloalkyl, —S(O)₂-heterocycloalkyl, —N(R²¹)-heterocycloalkyl, —C(O)—N(R²¹)-heterocycloalkyl, —N(R²¹)—C(O)-heterocycloalkyl, —N(R²¹)—C(O)—N(R²¹)-heterocycloalkyl, —N(R²¹)—S(O)-heterocycloalkyl, —N(R²¹)—S(O)₂-heterocycloalkyl, —N(R²¹)—S(O)₂—N(R²¹)-heterocycloalkyl, —S(O)—N(R²¹)-heterocycloalkyl, —S(O)₂—N(R²¹)-heterocycloalkyl,

(4c) cycloalkenyl, —O-cycloalkenyl, —C(O)-cycloalkenyl, —CO₂-cycloalkenyl, —S-cycloalkenyl, —S(O)-cycloalkenyl, —S(O)₂-cycloalkenyl, —N(R²¹)-cycloalkenyl, —C(O)—N(R²¹)-cycloalkenyl, —N(R²¹)—C(O)-cycloalkenyl, —N(R²¹)—C(O)—N(R²¹)-cycloalkenyl, —N(R²¹)—S(O)-cycloalkenyl, —N(R²¹)—S(O)₂-cycloalkenyl, —N(R²¹)—S(O)₂—N(R²¹)-cycloalkenyl, —S(O)—N(R²¹)-cycloalkenyl, —S(O)₂—N(R²¹)-cycloalkenyl,

(5c) heterocycloalkenyl, —O-heterocycloalkenyl, —C(O)-heterocycloalkenyl, —CO₂-heterocycloalkenyl, —S-heterocycloalkenyl, —S(O)-heterocycloalkenyl, —S(O)₂-heterocycloalkenyl, —N(R²¹)-heterocycloalkenyl, —C(O)—N(R²¹)-heterocycloalkenyl, and —N(R²¹)—C(O)-heterocycloalkenyl, —N(R²¹)—C(O)—N(R²¹)-heterocycloalkenyl, —N(R²¹)—S(O)-heterocycloalkenyl, —N(R²¹)—S(O)₂-heterocycloalkenyl, —N(R²¹)—S(O)₂—N(R²¹)-heterocycloalkenyl, —S(O)—N(R²¹)-heterocycloalkenyl, —S(O)₂—N(R²¹)-heterocycloalkenyl,

(6c) alkyl, —O-alkyl, —C(O)-alkyl, —CO₂-alkyl, —S-alkyl, —S(O)-alkyl, —S(O)₂-alkyl, —N(R²¹)-alkyl, —C(O)—N(R²¹)-alkyl, —N(R²¹)—C(O)-alkyl, —N(R²¹)—C(O)—N(R²¹)-alkyl, —N(R²¹)—S(O)-alkyl, —N(R²¹)—S(O)₂-alkyl, —N(R²¹)—S(O)₂—N(R²¹)-alkyl, —S(O)—N(R²¹)-alkyl, —S(O)₂—N(R²¹)-alkyl,

(7c) heteroalkyl, —O-heteroalkyl, —C(O)-heteroalkyl, —CO₂-heteroalkyl, —S-heteroalkyl, —S(O)-heteroalkyl, —S(O)₂-heteroalkyl, —N(R²¹)-heteroalkyl, —C(O)—N(R²¹)-heteroalkyl, —N(R²¹)—C(O)-heteroalkyl, —N(R²¹)—C(O)—N(R²¹)-heteroalkyl, —N(R²¹)—S(O)-heteroalkyl, —N(R²¹)—S(O)₂-heteroalkyl, —N(R²¹)—S(O)₂—N(R²¹)-heteroalkyl, —S(O)—N(R²¹)-heteroalkyl, —S(O)₂—N(R²¹)-heteroalkyl,

(8c) alkenyl, —O-alkenyl, —C(O)-alkenyl, —CO₂-alkenyl, —S-alkenyl, —S(O)-alkenyl, —S(O)₂-alkenyl, —N(R²¹)-alkenyl, —C(O)—N(R²¹)-alkenyl, —N(R²¹)—C(O)-alkenyl, —N(R²¹)—C(O)—N(R²¹)-alkenyl, —N(R²¹)—S(O)-alkenyl, —N(R²¹)—S(O)₂-alkenyl, —N(R²¹)—S(O)₂—N(R²¹)-alkenyl, —S(O)—N(R²¹)-alkenyl, —S(O)₂—N(R²¹)-alkenyl,

(10c) alkynyl, —O-alkynyl, —C(O)-alkynyl, —CO₂-alkynyl, —S-alkynyl, —S(O)-alkynyl, —S(O)₂-alkynyl, —N(R²¹)-alkynyl, —C(O)—N(R²¹)-alkynyl, —N(R²¹)—C(O)-alkynyl, —N(R²¹)—C(O)—N(R²¹)-alkynyl, —N(R²¹)—S(O)-alkynyl, —N(R²¹)—S(O)₂-alkynyl, —N(R²¹)—S(O)₂—N(R²¹)-alkynyl, —S(O)—N(R²¹)-alkynyl, —S(O)₂—N(R²¹)-alkynyl,

(12c) aryl, —O-aryl, —C(O)-aryl, —CO₂-aryl, —S-aryl, —S(O)-aryl, —S(O)₂-aryl, —N(R²¹)-aryl, —C(O)—N(R²¹)-aryl, —N(R²¹)—C(O)-aryl, —N(R²¹)—C(O)—N(R²¹)-aryl, —N(R²¹)—S(O)-aryl, —N(R²¹)—S(O)₂-aryl, —N(R²¹)—S(O)₂—N(R²¹)-aryl, —S(O)—N(R²¹)-aryl, —S(O)₂—N(R²¹)-aryl,

(13c) heteroaryl, —O-heteroaryl, —C(O)-heteroaryl, —CO₂-heteroaryl, —S-heteroaryl, —S(O)-heteroaryl, —S(O)₂-heteroaryl, —N(R²¹)-heteroaryl, —C(O)—N(R²¹)-heteroaryl, —N(R²¹)—C(O)-heteroaryl, —N(R²¹)—C(O)—N(R²¹)-heteroaryl, —N(R²¹)—S(O)-heteroaryl, —N(R²¹)—S(O)₂-heteroaryl, —N(R²¹)—S(O)₂—N(R²¹)-heteroaryl, —S(O)—N(R²¹)-heteroaryl, —S(O)₂—N(R²¹)-heteroaryl;

wherein said heteroalkyl, said heterocycloalkyl, said heterocycloalkenyl, and said heteroaryl of R² may be connected through any available carbon or heteroatom,

and wherein said heteroalkyl, said alkyl, said heterocycloalkyl, said cycloalkyl, said alkenyl, said heterocycloalkenyl, said cycloalkenyl, said aryl, said heteroaryl, and said alkynyl of R² are unsubstituted or substituted with one or more groups independently selected from are unsubstituted or substituted with one or more groups independently selected from (1a), (2a), (3a), (4a), (5a), (6a), (7a), (8a), (10a), (12a) and (13a) above;

or, alternatively, two R² groups attached to adjacent ring atoms of ring A are taken together to form a 5-6-membered aromatic or heteroaromatic ring;

or, alternatively, two R² groups attached to the same atom of ring A are taken together to form a moiety selected from the group consisting of carbonyl, spirocycloalkyl, spiroheteroalkyl, spirocycloalkenyl, spiroheterocycloalkenyl, oxime (the oxygen substituents of said oxime being independently selected from R¹⁵), and alkylidene (said alkylidene substituents being independently selected from R¹⁶), wherein said aryl and said heteroaryl of R² are unsubstituted or substituted with one or more groups independently selected from (1a), (2a), (3a), (4a), (5a), (6a), (7a), (8a), (10a), (12a) and (13a) above;

each R^{2A} (when present) is independently selected from the group consisting of:

(1e) cycloalkyl, —C(O)-cycloalkyl, —CO₂-cycloalkyl, —S(O)-cycloalkyl, —S(O)₂-cycloalkyl, —C(O)—N(R²¹)-cycloalkyl, —S(O)—N(R²¹)-cycloalkyl, —S(O)₂—N(R²¹)-cycloalkyl,

(2e) heterocycloalkyl, —C(O)-heterocycloalkyl, —CO₂-heterocycloalkyl, —S(O)-heterocycloalkyl, —S(O)₂-heterocycloalkyl, —C(O)—N(R²¹)-heterocycloalkyl, —S(O)—N(R²¹)-heterocycloalkyl, —S(O)₂—N(R²¹)-heterocycloalkyl,

(3e) cycloalkenyl, —C(O)-cycloalkenyl, —CO₂-cycloalkenyl, —S(O)-cycloalkenyl, —S(O)₂-cycloalkenyl, —C(O)—N(R²¹)-cycloalkenyl, —S(O)—N(R²¹)-cycloalkenyl, —S(O)₂—N(R²¹)-cycloalkenyl,

(4e) heterocycloalkenyl, —C(O)-heterocycloalkenyl, —CO₂-heterocycloalkenyl, —S(O)-heterocycloalkenyl, —S(O)₂-heterocycloalkenyl, —C(O)—N(R²¹)-heterocycloalkenyl, —S(O)—N(R²¹)-heterocycloalkenyl, —S(O)₂—N(R²¹)-heterocycloalkenyl,

(5e) alkyl, —C(O)-alkyl, —CO₂-alkyl, —S(O)-alkyl, —S(O)₂-alkyl, —C(O)—N(R²¹)-alkyl, —S(O)—N(R²¹)-alkyl, —S(O)₂—N(R²¹)-alkyl,

(6e) heteroalkyl, —C(O)-heteroalkyl, —CO₂-heteroalkyl, —S(O)-heteroalkyl, —S(O)₂-heteroalkyl, —C(O)—N(R²¹)-heteroalkyl, —S(O)—N(R²¹)-heteroalkyl, —S(O)₂—N(R²¹)-heteroalkyl,

(7e) alkenyl, —C(O)-alkenyl, —CO₂-alkenyl, —S(O)-alkenyl, —S(O)₂-alkenyl, —C(O)—N(R²¹)-alkenyl, —S(O)—N(R²¹)-alkenyl, —S(O)₂—N(R²¹)-alkenyl,

(9e) alkynyl, —C(O)-alkynyl, —CO₂-alkynyl, —S(O)-alkynyl, —S(O)₂-alkynyl, —C(O)—N(R²¹)-alkynyl, —S(O)—N(R²¹)-alkynyl, —S(O)₂—N(R²¹)-alkynyl,

(11e) aryl, —C(O)-aryl, —CO₂-aryl, —S(O)-aryl, —S(O)₂-aryl, —C(O)—N(R²¹)-aryl, —S(O)—N(R²¹)-aryl, —S(O)₂—N(R²¹)-aryl,

(12e) heteroaryl, —C(O)-heteroaryl, —CO₂-heteroaryl, —S(O)-heteroaryl, —S(O)₂-heteroaryl, —C(O)—N(R²¹)-heteroaryl, —S(O)—N(R²¹)-heteroaryl, —S(O)₂—N(R²¹)-heteroaryl,

(13e) —CHO;

wherein said heteroalkyl, said heterocycloalkyl, said heterocycloalkenyl, and said heteroaryl of R^{2A} may be connected through any available carbon or heteroatom,

and wherein said heteroalkyl, said alkyl, said heterocycloalkyl, said cycloalkyl, said alkenyl, said heterocycloalkenyl, said cycloalkenyl, said aryl, said heteroaryl, and said alkynyl of R^{2A} are unsubstituted or substituted with one or more groups independently selected from are unsubstituted or substituted with one or more groups independently selected from (1a), (2a), (3a), (4a), (5a), (6a), (7a), (8a), (10a), (12a) and (13a) above;

R³ is selected from H and lower alkyl;

Z is a moiety selected from —(C(R¹¹)₂)—(C(R¹²R¹³))ₘ—C(O)OH, —(C(R¹¹)₂)—(C(R¹⁴)₂)ₙ—C(O)OH, from —(C(R¹¹)₂)—(C(R¹²R¹³))ₘ—C(O)Oalkyl, —(C(R¹¹)₂)—(C(R¹⁴)₂)ₙ—C(O)Oalkyl,

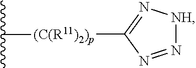

—(C(R¹¹)₂)—(C(R¹²R¹³))ₘ-Q, and —(C(R¹¹)₂)—(C(R¹⁴)₂)ₙ-Q,

wherein Q is a moiety selected from the group consisting of:

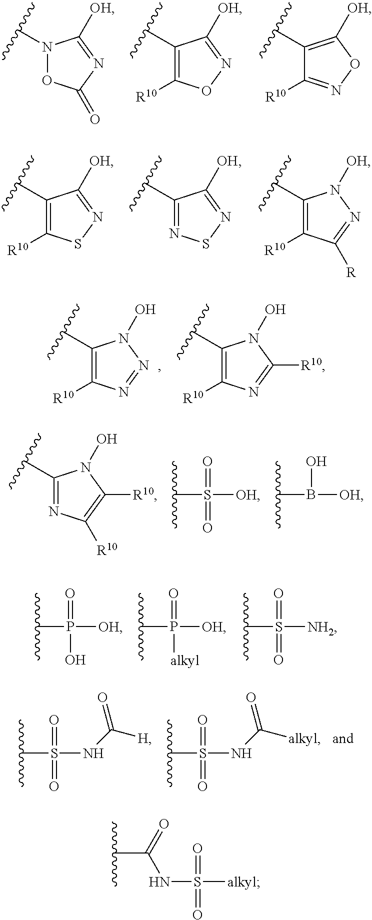

m is an integer from 0 to 5;

n is an integer from 0 to 5;

p is an integer from 0 to 5;

each R⁴ is independently selected from H, —OH, lower alkyl, haloalkyl, alkoxy, heteroalkyl, cyano-substituted lower alkyl, hydroxy-substituted lower alkyl, cycloalkyl, β-cycloalkyl, —O-alkyl-cycloalkyl, and heterocycloalkyl, —O-heterocycloalkyl, and —O-alkyl-heterocycloalkyl;

each R^{5A} is independently selected from H, alkyl, haloalkyl, heteroalkyl, cyano-substituted alkyl, hydroxy-substituted alkyl, cycloalkyl, -alkyl-cycloalkyl, and heterocycloalkyl, -alkyl-heterocycloalkyl,

or, alternatively, two R^{5A} groups are taken together with the carbon atom to which they are attached to form a carbonyl group, a spirocycloalkyl group, a spiroheterocycloalkyl group, an oxime group, or a substituted oxime group (said oxime substituents being independently selected from alkyl, haloalkyl, hydroxyl-substituted alkyl, and cycloalkyl);

each R⁵ is independently selected from H, —OH, alkyl, haloalkyl, alkoxy, heteroalkyl, cyano-substituted alkyl, hydroxy-substituted alkyl, cycloalkyl, -alkyl-cycloalkyl, —O-cycloalkyl, —O-alkyl-cycloalkyl, and heterocycloalkyl, -alkyl-heterocycloalkyl, —O-heterocycloalkyl, and —O-alkyl-heterocycloalkyl,

or, alternatively, two R⁵ groups bound to the same carbon atom are taken together with the carbon atom to which they are attached to form a carbonyl group, a spirocycloalkyl group, a spiroheterocycloalkyl group, an oxime group, or a substituted oxime group (said oxime substituents being independently selected from alkyl, haloalkyl, hydroxyl-substituted alkyl, and cycloalkyl);

each R⁷ is independently selected from H, alkyl, haloalkyl, heteroalkyl, alkenyl, and alkynyl;

each R¹⁰ is independently selected from H and alkyl;

each R¹¹ is independently selected from H and lower alkyl;

each R¹² is independently selected from H, lower alkyl, —OH, hydroxy-substituted lower alkyl;

each R¹³ is independently selected from H, unsubstituted lower alkyl, lower alkyl substituted with one or more groups each independently selected from hydroxyl and alkoxy, or R¹² and R¹³ are taken together to form an oxo;

each R¹⁴ is independently selected from H and fluoro;

each R¹⁵ is independently selected from H, alkyl, haloalkyl, heteroalkyl, heterocycloalkyl, and cycloalkyl;

each R¹⁶ is independently selected from H, alkyl, haloalkyl, heteroalkyl, heterocycloalkyl, cycloalkyl, aryl, and heteroaryl;

each R²⁰ is independently selected from H, alkyl, haloalkyl, heteroalkyl, alkenyl, and alkynyl;

and each R²¹ is independently selected from:

(1d) hydrogen,

(2d) cycloalkyl, —C(O)-cycloalkyl, —CO₂-cycloalkyl, —S(O)-cycloalkyl, —S(O)₂-cycloalkyl, —C(O)—N(R²⁰)-cycloalkyl, —S(O)—N(R²⁰)-cycloalkyl, —S(O)₂—N(R²⁰)-cycloalkyl,

(3d) heterocycloalkyl, —C(O)-heterocycloalkyl, —CO₂-heterocycloalkyl, —S(O)-heterocycloalkyl, —S(O)₂-heterocycloalkyl, —C(O)—N(R²⁰)-heterocycloalkyl, —S(O) —N(R²⁰)-heterocycloalkyl, —S(O)₂—N(R²⁰)-heterocycloalkyl,

(4d) cycloalkenyl, —C(O)-cycloalkenyl, —CO₂-cycloalkenyl, —S(O)-cycloalkenyl, —S(O)₂-cycloalkenyl, —C(O)—N(R²⁰)-cycloalkenyl, —S(O)—N(R²⁰)-cycloalkenyl, —S(O)₂—N(R²⁰)-cycloalkenyl,

(5d) heterocycloalkenyl, —C(O)-heterocycloalkenyl, —CO₂-heterocycloalkenyl, —S(O)-heterocycloalkenyl, —S(O)₂-heterocycloalkenyl, —C(O)—N(R²⁰)-heterocycloalkenyl, —S(O)—N(R²⁰)-heterocycloalkenyl, —S(O)₂—N(R²⁰)-heterocycloalkenyl,

(6d) alkyl, —C(O)-alkyl, —CO₂-alkyl, —S(O)-alkyl, —S(O)₂-alkyl, —C(O)—N(R²⁰)-alkyl, —S(O)—N(R²⁰)-alkyl, —S(O)₂—N(R²⁰)-alkyl,

(7d) heteroalkyl, —C(O)-heteroalkyl, —CO₂-heteroalkyl, —S(O)-heteroalkyl, —S(O)₂-heteroalkyl, —C(O)—N(R²⁰)-heteroalkyl, —S(O)—N(R²⁰)-heteroalkyl, —S(O)₂—N(R²⁰)-heteroalkyl,

(8d) alkenyl, —C(O)-alkenyl, —CO₂-alkenyl, —S(O)-alkenyl, —S(O)₂-alkenyl, —C(O)—N(R²⁰)-alkenyl, —S(O)—N(R²⁰)-alkenyl, —S(O)₂—N(R²⁰)-alkenyl,

(10d) alkynyl, —C(O)-alkynyl, —CO₂-alkynyl, —S(O)-alkynyl, —S(O)₂-alkynyl, —C(O)—N(R²⁰)-alkynyl, —S(O)—N(R²⁰)-alkynyl, —S(O)₂—N(R²⁰)-alkynyl,

(12d) aryl, —O-aryl, —C(O)-aryl, —CO₂-aryl, —S(O)-aryl, —S(O)₂-aryl, —C(O)—N(R²⁰)-aryl, —S(O)—N(R²⁰)-aryl, —S(O)₂—N(R²⁰)-aryl,

(13d) heteroaryl, —O-heteroaryl, —C(O)-heteroaryl, —CO₂-heteroaryl, —S(O)-heteroaryl, —S(O)₂-heteroaryl, —C(O)—N(R²⁰)-heteroaryl, —S(O)—N(R²⁰)-heteroaryl, —S(O)₂—N(R²⁰)-heteroaryl;

wherein said heteroalkyl, said heterocycloalkyl, said heterocycloalkenyl, and said heteroaryl of R²¹ may be connected through any available carbon or heteroatom,

and wherein said alkyl, said heteroalkyl, said alkenyl, said cycloalkyl, said heterocycloalkyl, said cycloalkenyl, said heterocycloalkenyl, said aryl, said heteroaryl, and said alkynyl of R²¹ are unsubstituted or substituted with one or more groups independently selected from (1a), (2a), (3a), (4a), (5a), (6a), (7a), (8a), (10a), (12a) and (13a) above.

In one embodiment, in Formula (A), ring A represents a 3-8-membered spirocycloalkyl or spirocycloalkenyl ring.

In one embodiment, in Formula (A), ring A represents a 3-8-membered spirocycloalkyl or spirocycloalkenyl ring, which ring is substituted with from 1 to 5 independently selected R² groups, which R² groups may be attached to the same or different ring carbon atom(s).

In one embodiment, in Formula (A), ring A represents a 3-8-membered spirocycloalkyl or spirocycloalkenyl ring, which ring is substituted with from 1 to 3 independently selected R² groups, which R² groups may be attached to the same or different ring carbon atom(s).

In one embodiment, in Formula (A), ring A represents a 3-8-membered spirocycloalkyl or spirocycloalkenyl ring, which ring is substituted with from 1 to 2 independently selected R² groups, which R² groups may be attached to the same or different ring carbon atom(s).

In one embodiment, in Formula (A), ring A represents a 3-8-membered spirocycloalkyl or spirocycloalkenyl ring, which ring is substituted with 1 R² group.

In one embodiment, in Formula (A), ring A represents a 5-7-membered spirocycloalkyl or spirocycloalkenyl ring.

In one embodiment, in Formula (A), ring A represents a 5-7-membered spirocycloalkyl or spirocycloalkenyl ring, which ring is substituted with from 1 to 5 independently selected R² groups, which R² groups may be attached to the same or different ring carbon atom(s).

In one embodiment, in Formula (A), ring A represents a 5-7-membered spirocycloalkyl or spirocycloalkenyl ring, which ring is substituted with from 1 to 3 independently selected R² groups, which R² groups may be attached to the same or different ring carbon atom(s).

In one embodiment, in Formula (A), ring A represents a 5-7-membered spirocycloalkyl or spirocycloalkenyl ring, which ring is substituted with from 1 to 2 independently selected R² groups, which R² groups may be attached to the same or different ring carbon atom(s).

In one embodiment, in Formula (A), ring A represents a 5-7-membered spirocycloalkyl or spirocycloalkenyl ring, which ring is substituted with 1 R² group.

Non-limiting examples of ring A when ring A represents a spirocycloalkyl ring, which may be unsubstituted or substituted as described herein, include: spirocyclobutyl, spirocyclopentyl, spirocyclohexyl, spirocycloheptyl, spirocyclooctyl, spironorbornanyl, and spiroadamantanyl.

Non-limiting examples of ring A when ring A represents a spirocycloalkenyl ring, which may be unsubstituted or substituted as described herein, include partially or fully unsaturated versions of the spirocycloalkyl moieties described above. Non-limiting examples include: spirocyclopentenyl, spirocyclohexenyl, spirocycloheptenyl, and spirocyclooctenyl.

In one embodiment, in Formula (A), ring A represents a 3-8-membered spiroheterocycloalkyl ring containing up to 3 ring heteroatoms, 1-3 of which are selected from O, S, S(O), S(O)₂, and N or N-oxide.

In one embodiment, in Formula (A), ring A represents a 3-8-membered spiroheterocycloalkenyl ring containing up to 3 ring heteroatoms, 1-3 of which are selected from O, S, S(O), S(O)₂, and N or N-oxide.

In one embodiment, in Formula (A), ring A represents a 3-8-membered spiroheterocycloalkyl ring containing up to 3 ring heteroatoms, 0-1 of which are O, S, 8(O), and 5(O)₂, and 1-2 of which are N or N-oxide, which ring A is substituted on one or more available ring carbon atom(s) with from 0 to 5 independently selected R² groups, and which ring A is optionally further substituted on one or more available ring nitrogen atoms with from 0 to 2 independently selected R^{2A} groups.

In one embodiment, in Formula (A), ring A represents a 3-8-membered spiroheterocycloalkenyl ring containing up to 3 ring heteroatoms, 0-1 of which are 0, S, S(O), and 5(O)₂, and 1-2 of which are N or N-oxide, which ring A is substituted on one or more available ring carbon atom(s) with from 0 to 5 independently selected R² groups, and which ring A is optionally further substituted on one or more available ring nitrogen atoms with 0 to 2 independently selected R^{2A} groups.

In one embodiment, in Formula (A), ring A represents a 5-7-membered spiroheterocycloalkyl ring containing up to 3 ring heteroatoms, 0-1 of which are O, S, S(O), and S(O)₂, and 1-2 of which are N or N-oxide, which ring A is substituted on one or more available ring carbon atom(s) with from 0 to 5 independently selected R² groups, and which ring A is optionally further substituted on one or more available ring nitrogen atoms with 0 to 2 independently selected R^{2A} groups.

In one embodiment, in Formula (A), ring A represents a 5-7-membered spiroheterocycloalkenyl ring containing up to 3 ring heteroatoms, 0-1 of which are O, S, S(O), and S(O)₂, and 1-2 of which are N or N-oxide, which ring A is substituted on one or more available ring carbon atom(s) with from 0 to 5 independently selected R² groups, and which ring A is optionally further substituted on one or more available ring nitrogen atoms with 0 to 2 independently selected R^{2A} groups.

In one embodiment, in Formula (A), ring A represents a spiropiperidinyl ring.

In one embodiment, in Formula (A), ring A represents a spiropiperidinyl ring, which ring A is substituted on one or more available ring carbon atom(s) with from 0 to 5 independently selected R² groups, and which ring A is optionally further substituted on the spiropiperidinyl nitrogen with R.

In one embodiment, in Formula (A), ring A represents a spiropiperidinyl ring, which ring A is substituted on one or more available ring carbon atom(s) with from 0 to 3 independently selected R² groups.

In one embodiment, in Formula (A), ring A represents a spiropiperidinyl ring, which ring A is substituted on one or more available ring carbon atom(s) with from 0 to 2 independently selected R² groups.

In one embodiment, in Formula (A), ring A represents a spiropiperidinyl ring, which ring A is substituted on one or more available ring carbon atom(s) with an R² group.

In one embodiment, in Formula (A), ring A represents a spiropiperidinyl ring, which ring A is substituted on the spiropiperidinyl nitrogen with R^{2A}.

In one embodiment, in Formula (A), two R² groups are attached to the same atom of ring A and are taken together with said atom of ring A to form an oxime group. In such embodiments, said oxime group, when present, is shown attached to the compounds of Formula (A) as follows:

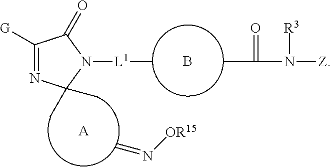

In one embodiment, in Formula (A), two R² groups are attached to the same atom of ring A and are taken together with said atom of ring A to form an alkylidene group. In such embodiments, said alkylidene group, when present, is shown attached to the compounds of Formula (A) as follows:

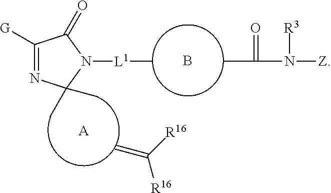

Additional non-limiting examples of ring A when ring A represents a spiroheterocycloalkyl ring, which may be unsubstituted or substituted as described herein, include: spiropyrrolidinyl, spirodioxolanyl, spiroimidazolidinyl, spiropyrazolidinyl, spiropiperidinyl, spirodioxanyl, spiromorpholinyl, spirotetrahydropyranyl, spirodithianyl, spirothiomorpholinyl, spiropiperazinyl, and spirotrithianyl.

Additional non-limiting examples of ring A when ring A represents a spiroheterocycloalkenyl ring, which may be unsubstituted or substituted as described herein, include unsaturated versions of the following moieties spiropyrrolidinyl, spirodioxolanyl, spiroimidazolidinyl, spiropyrazolidinyl, spiropiperidinyl, spirodioxanyl, spiromorpholinyl, spirodithianyl, spirothiomorpholinyl, spiropiperazinyl, and spirotrithianyl.

In one embodiment, the compounds of the invention have the general structure shown in Formula (A-1):

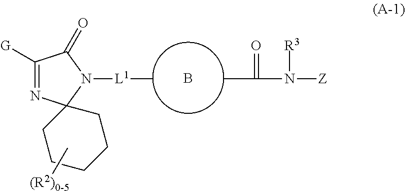

and include pharmaceutically acceptable salts, solvates, esters, prodrugs, tautomers, and isomers of said compounds,

wherein ring B, G, L¹, R³, Z and each R² are selected independently of each other and as defined in Formula (A).

In one embodiment, in Formula (A-1), two R² groups are attached to the same atom of ring A and are taken together with said atom of ring A to form an oxime group, wherein said compound has the general structure:

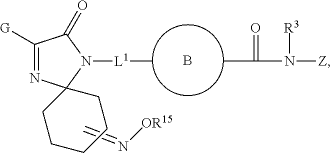

wherein G, L¹, R¹⁵, ring B, R³, and Z are each as defined in formula (A).

In one embodiment, in Formula (A-1), two R² groups are attached to the same atom of ring A and are taken together with said atom of ring A to form an alkylidene group, wherein said compound has the general structure:

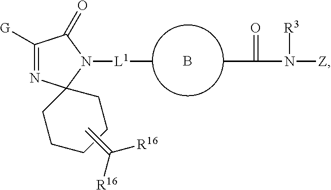

wherein G, L¹, each R¹⁶, ring B, R³, and Z are each as defined in formula (A).

In one embodiment, the compounds of the invention have the general structure shown in Formula (A-1a):

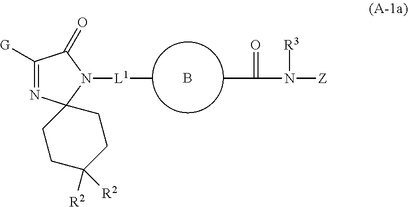

and include pharmaceutically acceptable salts, solvates, esters, prodrugs, tautomers, and isomers of said compounds,

wherein ring B, G, L¹, R³, Z and each R² are selected independently of each other and as defined in Formula (A).

In one embodiment, the compounds of the invention have the general structure shown in Formula (A-1b):

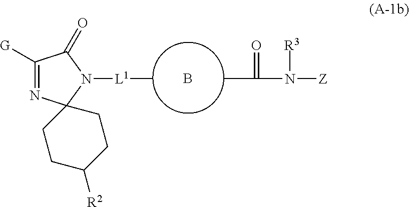

and include pharmaceutically acceptable salts, solvates, esters, prodrugs, tautomers, and isomers of said compounds,

wherein ring B, G, L¹, R³, Z and each R² are selected independently of each other and as defined in Formula (A).

In one embodiment, the compounds of the invention have the general structure shown in Formula (A-2a):

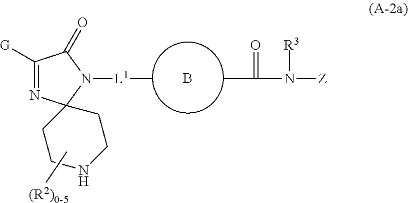

and include pharmaceutically acceptable salts, solvates, esters, prodrugs, tautomers, and isomers of said compounds,

wherein ring B, G, L¹, R³, Z and each R² are selected independently of each other and as defined in Formula (A).

In one embodiment, the compounds of the invention have the general structure shown in Formula (A-2b):

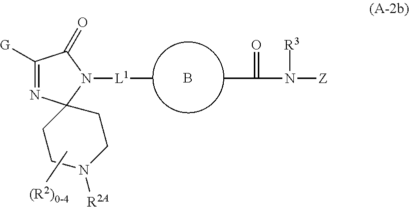

and include pharmaceutically acceptable salts, solvates, esters, prodrugs, tautomers, and isomers of said compounds,

wherein ring B, G, L¹, R³, Z, R^{2A} and each R² are selected independently of each other and as defined in Formula (A).

In one embodiment, the compounds of the invention have the general structure shown in Formula (A-2c):

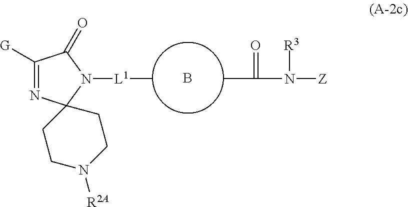

and include pharmaceutically acceptable salts, solvates, esters, prodrugs, tautomers, and isomers of said compounds,

wherein ring B, G, L¹, R³, Z and R^{2A} are selected independently of each other and as defined in Formula (A).

In one embodiment, the compounds of the invention have the general structure shown in Formula (A-2d):

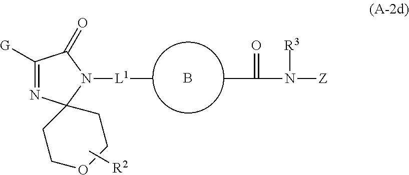

and include pharmaceutically acceptable salts, solvates, esters, prodrugs, tautomers, and isomers of said compounds,

wherein ring B, G, L¹, R³, Z and each R² are selected independently of each other and as defined in Formula (A).

In one embodiment, in each of Formula (A), Formula (A-1), Formula (A-1a), Formula (A-1b), Formula (A-2a), Formula (A-2b), Formula (A-2c), and Formula (A-2d), ring B is a phenyl ring wherein the -L¹- and the —C(O)N(R³)Z moieties shown in the formula are bound to said phenyl ring in a 1,4-relationship, and wherein said phenyl ring is (in addition to the -L¹- and —C(O)N(R³)—Z moieties shown) optionally further substituted with one or more substituents R^{a}, wherein each R^{a} (when present) is independently selected from the group consisting of halo, alkyl, and haloalkyl.

In one embodiment, in each of Formula (A), Formula (A-1), Formula (A-1a), Formula (A-1 b), Formula (A-2a), Formula (A-2b), Formula (A-2c), and Formula (A-2d), ring B is a 5-membered heteroaromatic ring containing from 1 to 3 ring heteroatoms independently selected from N, O, and S, wherein the -L¹- and the —C(O)N(R³)—Z moieties shown in the formula are bound to said 5-membered ring in a 1,3-relationship, and wherein said 5-membered heteroaromatic ring is (in addition to the -L¹- and —C(O)N(R³)—Z moieties shown) optionally further substituted with one or more substituents R^{a}, wherein each R^{a} (when present) is independently selected from the group consisting of halo, alkyl, and haloalkyl.

In one embodiment, in each of Formula (A), Formula (A-1), Formula (A-1a), Formula (A-1b), Formula (A-2a), Formula (A-2b), Formula (A-2c), and Formula (A-2d), ring B is a 6-membered heteroaromatic ring containing from 1 to 3 ring nitrogen atoms, wherein the -L¹- and the —C(O)N(R³)—Z moieties shown in the formula are bound to said 6-membered ring in a 1,4-relationship, and wherein said 6-membered heteroaromatic ring is (in addition to -L¹- and —C(O)N(R³)Z moieties shown) optionally further substituted with one or more substituents R^{a}, wherein each R^{a} (when present) is independently selected from the group consisting of halo, alkyl, and haloalkyl.

In one embodiment, in each of Formula (A), Formula (A-1), Formula (A-1a), Formula (A-1b), Formula (A-2a), Formula (A-2b), Formula (A-2c), and Formula (A-2d), ring B is phenyl.

In one embodiment, in each of Formula (A), Formula (A-1), Formula (A-1a), Formula (A-1b), Formula (A-2a), Formula (A-2b), Formula (A-2c), and Formula (A-2d), ring B is phenyl which, in addition to the moieties -L¹- and —C(O)N(R³)—Z shown in the formula, is further substituted with one or more independently selected R^{a} groups.

In one embodiment, in each of Formula (A), Formula (A-1), Formula (A-1a), Formula (A-1b), Formula (A-2a), Formula (A-2b), Formula (A-2c), and Formula (A-2d), ring B is a phenyl which, in addition to the moieties -L¹- and —C(O)N(R³)—Z shown in the formula, is further substituted with from 1 to 2 substituents, each independently selected from halo, alkyl, and haloalkyl.

In one embodiment, in each of Formula (A), Formula (A-1), Formula (A-1a), Formula (A-1b), Formula (A-2a), Formula (A-2b), Formula (A-2c), and Formula (A-2d), ring B is a 5-membered heteroaromatic ring having from 1 to 3 ring heteroatoms independently selected from N, O, and S, wherein said ring B is not further substituted.

In one embodiment, in each of Formula (A), Formula (A-1), Formula (A-1a), Formula (A-1b), Formula (A-2a), Formula (A-2b), Formula (A-2c), and Formula (A-2d), ring B is a 6-membered heteroaromatic ring having from 1 to 3 ring nitrogen atoms, wherein said ring B is not further substituted.

In one embodiment, in each of Formula (A), Formula (A-1), Formula (A-1a), Formula (A-1 b), Formula (A-2a), Formula (A-2b), Formula (A-2c), and Formula (A-2d), ring B is a 5-membered heteroaromatic ring having from 1 to 3 ring heteroatoms independently selected from N, O, and S, wherein said ring B is further substituted with one or more substituents. Said further substituents in such embodiments may be bound to one or more available ring carbon atoms and/or ring nitrogen atoms.

In one embodiment, in each of Formula (A), Formula (A-1), Formula (A-1a), Formula (A-1b), Formula (A-2a), Formula (A-2b), Formula (A-2c), and Formula (A-2d), ring B is a 6-membered heteroaromatic ring having from 1 to 3 ring nitrogen atoms wherein said ring B is further substituted with one or more substituents. Said further substituents in such embodiments may be bound to one or more available ring carbon atoms and/or ring nitrogen atoms.

In one embodiment, in each of Formula (A), Formula (A-1), Formula (A-1a), Formula (A-1b), Formula (A-2a), Formula (A-2b), Formula (A-2c), and Formula (A-2d), ring B is a 5-membered heteroaromatic ring having from 1 to 3 ring heteroatoms independently selected from N, O, and S, wherein said 5-membered heteroaromatic ring is further substituted with from 1 to 2 substituents, each substituent being independently selected from halo, alkyl, and haloalkyl. In one such embodiment, ring B contains two said substituents. In another such embodiment, ring B contains one said substitutent.

In one embodiment, in each of Formula (A), Formula (A-1), Formula (A-1a), Formula (A-1b), Formula (A-2a), Formula (A-2b), Formula (A-2c), and Formula (A-2d), ring B is a 5-membered heteroaromatic ring, non-limiting examples of such rings include, but are not limited to: furan, thiophene, pyrrole, imidazole, pyrazole, 1,2,3-triazole, 1,2,4-triazole, thiazole, thiadiazole, oxazole, oxadiazole, and isoxazole, each of which may be optionally further substituted as described herein. Non-limiting examples of ring B (shown connected to moieties L¹ and —C(O)—N(R³)—Z) include:

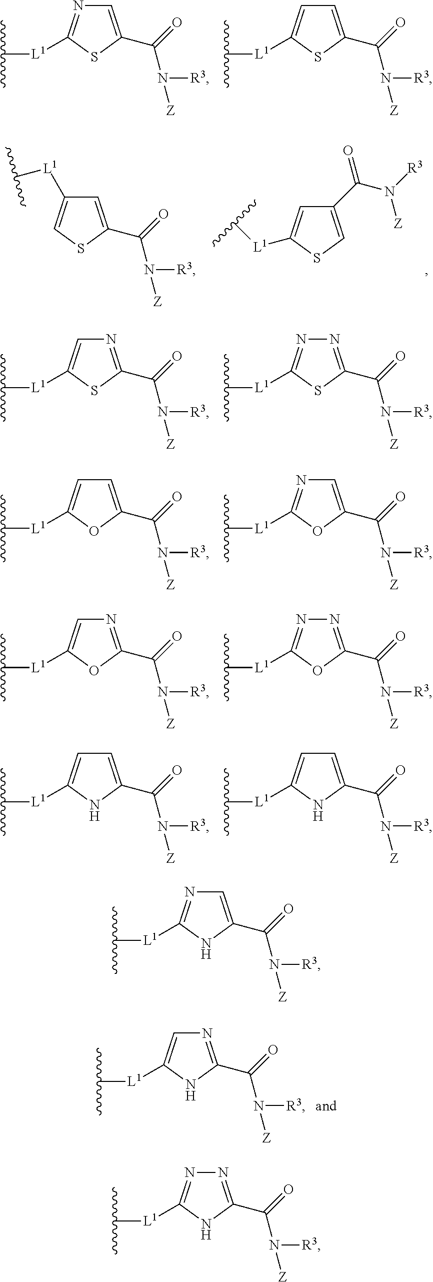

wherein each ring B shown is optionally further substituted on an available ring carbon atom or ring nitrogen atom with one or more groups R^{a}, wherein each R^{a}, when attached to a ring carbon atom, is independently selected from halo, alkyl, and haloalkyl, and wherein each R^{a}, when attached to a ring nitrogen atom, is independently selected from alkyl, and haloalkyl. Non-limiting examples of such groups substituted on an available ring nitrogen atom include:

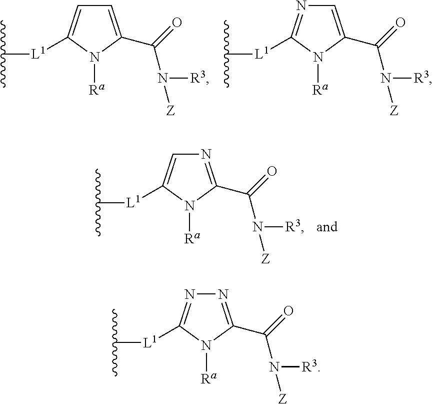

In one embodiment, in each of Formula (A), Formula (A-1), Formula (A-1a), Formula (A-1b), Formula (A-2a), Formula (A-2b), Formula (A-2c), and Formula (A-2d), ring B is a 6-membered heteroaromatic ring having from 1 to 3 ring nitrogen atoms, wherein said ring B is further substituted with from 1 to 3 substituents, each substituent being independently selected from halo, alkyl, and haloalkyl. In one such embodiment, ring B contains three said substituents. In one such embodiment, ring B contains two said substituents. In another such embodiment, ring B contains one said substitutent.

When, in each of Formula (A), Formula (A-1), Formula (A-1a), Formula (A-1 b), Formula (A-2a), Formula (A-2b), Formula (A-2c), and Formula (A-2d), ring B is a 6-membered heteroaromatic ring, non-limiting examples of such rings include: pyridine, pyrimidine, pyrazine, pyridazine, and triazine, each of which may be optionally further substituted as described herein. Non-limiting examples of ring B (shown connected to moieties L¹ and —C(O)—N(R³)—Z) include:

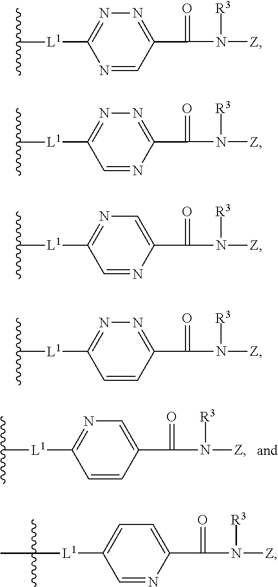

wherein any of such moieties may be optionally further substituted with one or more groups R^{a}, wherein each R^{a} is independently selected from halo, alkyl, and haloalkyl.

In the various embodiments of the compounds of the invention described herein, functional groups for L¹ are to be read from left to right unless otherwise stated.

In one embodiment, in each of Formula (A), Formula (A-1), Formula (A-1a), Formula (A-1b), Formula (A-2a), Formula (A-2b), Formula (A-2c), and Formula (A-2d), L¹ is selected from the group consisting of: a bond, —N(R⁴)—, —N(R⁴)—(C(R^{5A})₂)—, —O—, —O—(C(R^{5A})₂)—, and —(C(R^{5A})₂)—(C(R⁵)₂)ₛ—, wherein s is an integer from 0 to 3.

In one embodiment, in each of Formula (A), Formula (A-1), Formula (A-1a), Formula (A-1b), Formula (A-2a), Formula (A-2b), Formula (A-2c), and Formula (A-2d), L¹ is selected from the group consisting of: a bond and —(C(R^{5A})₂)—(C(R⁵)₂)ₛ—, wherein s is an integer from 0 to 1, and wherein each R⁵ and each R^{5A} is independently selected from the group consisting of H, lower alkyl, -lower alkyl-Si(CH₃)₃, lower haloalkyl, and lower alkyl substituted with one or more groups independently selected from hydroxyl and cyano. In one such embodiment, s is 0. In one such embodiment, s is 1.

In one embodiment, in each of Formula (A), Formula (A-1), Formula (A-1a), Formula (A-1b), Formula (A-2a), Formula (A-2b), Formula (A-2c), and Formula (A-2d), L¹ is selected from the group consisting of lower branched alkyl and -lower alkyl-Si(CH₃)₃.

In one embodiment, in each of Formula (A), Formula (A-1), Formula (A-1a), Formula (A-1b), Formula (A-2a), Formula (A-2b), Formula (A-2c), and Formula (A-2d), L¹ is a bond.

In one embodiment, in each of Formula (A), Formula (A-1), Formula (A-1a), Formula (A-1b), Formula (A-2a), Formula (A-2b), Formula (A-2c), and Formula (A-2d), L¹ is —N(R⁴)—(C(R^{5A})₂)—, wherein each R^{5A} is independently selected from H, lower alkyl, lower haloalkyl, and lower alkyl substituted with one or more hydroxyl and R⁴ is selected from H and lower alkyl.

In one embodiment, in each of Formula (A), Formula (A-1), Formula (A-1a), Formula (A-1b), Formula (A-2a), Formula (A-2b), Formula (A-2c), and Formula (A-2d), L¹ is —O—(C(R^{5A})₂)—, wherein each R^{5A} is independently selected from H, lower alkyl, lower haloalkyl, and lower alkyl substituted with one or more hydroxyl.

In one embodiment, in each of Formula (A), Formula (A-1), Formula (A-1a), Formula (A-1b), Formula (A-2a), Formula (A-2b), Formula (A-2c), and Formula (A-2d), L¹ is selected from the group consisting of a bond, —NH—(CH₂)₂—, —O—(CH₂)₂—, —O—, —NH—, —N(CH₃)—, —CH₂—, —CH(CH₃)—, and —CH₂CH₂—.

In one embodiment, in each of Formula (A), Formula (A-1), Formula (A-1a), Formula (A-1b), Formula (A-2a), Formula (A-2b), Formula (A-2c), and Formula (A-2d), L¹ is selected from the group consisting of —CH₂—, —CH(CH₃)—, and —CH₂CH₂—.

In one embodiment, in each of Formula (A), Formula (A-1), Formula (A-1a), Formula (A-1b), Formula (A-2a), Formula (A-2b), Formula (A-2c), and Formula (A-2d), L¹ is selected from the group consisting of: —CH(cycloalkylalkyl)- and —CH(heterocycloalkylalkyl)-.

In one embodiment, in each of Formula (A), Formula (A-1), Formula (A-1a), Formula (A-1 b), Formula (A-2a), Formula (A-2b), Formula (A-2c), and Formula (A-2d), L¹ is —C(R^{5A})₂—, wherein each R^{5A} is independently selected from the group consisting of H, lower alkyl, -lower alkyl-Si(CH₃)₃, haloalkyl, heteroalkyl, cyano-substituted lower alkyl, hydroxy-substituted lower alkyl, cycloalkyl, cycloalkylalkyl-, heterocycloalkyl, and heterocycloalkylalkyl-.

In one embodiment, in each of Formula (A), Formula (A-1), Formula (A-1a), Formula (A-1 b), Formula (A-2a), Formula (A-2b), Formula (A-2c), and Formula (A-2d), L¹ is —CH(R^{5A})—, wherein R^{5A} is selected from the group consisting of H, lower alkyl, -lower alkyl-Si(CH₃)₃, haloalkyl, heteroalkyl, cyano-substituted lower alkyl, hydroxy-substituted lower alkyl, cycloalkyl, cycloalkylalkyl-, heterocycloalkyl, and heterocycloalkylalkyl-.

In one embodiment, in each of Formula (A), Formula (A-1), Formula (A-1a), Formula (A-1 b), Formula (A-2a), Formula (A-2b), Formula (A-2c), and Formula (A-2d), L¹ is selected from the group consisting of:

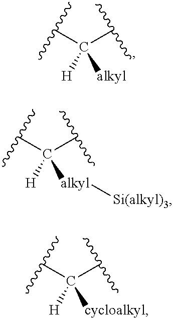

### and —(CH₂)₁₋₃—.

In one embodiment, in each of Formula (A), Formula (A-1), Formula (A-1a), Formula (A-1b), Formula (A-2a), Formula (A-2b), Formula (A-2c), and Formula (A-2d), L¹ is selected from the group consisting of

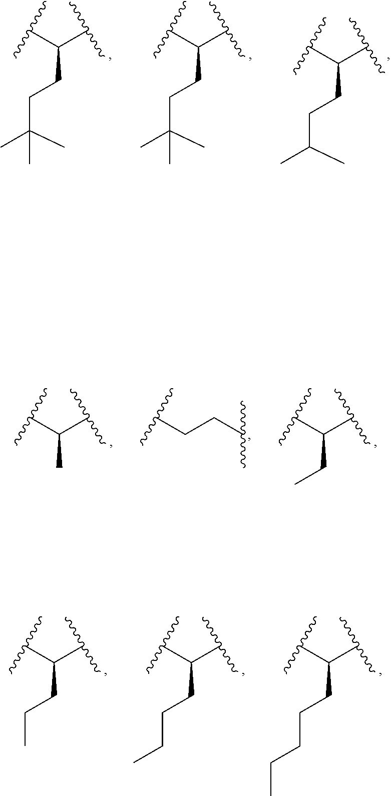

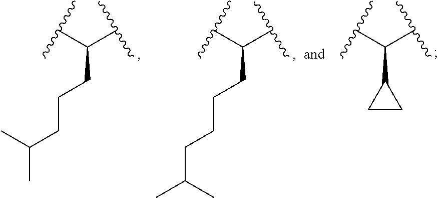

In one embodiment, in each of Formula (A), Formula (A-1), Formula (A-1a), Formula (A-1b), Formula (A-2a), Formula (A-2b), Formula (A-2c), and Formula (A-2d), L¹ is selected from the group consisting of

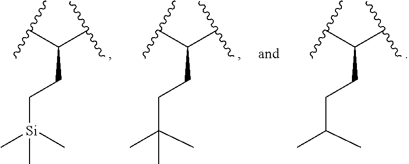

In one embodiment, in each of Formula (A), Formula (A-1), Formula (A-1a), Formula (A-1b), Formula (A-2a), Formula (A-2b). Formula (A-2c) and Formula IA-9d), L¹ is selected from the group consisting of

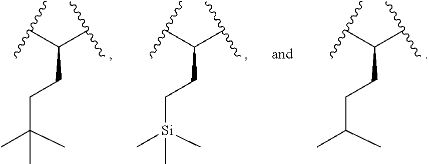

In one embodiment, in each of Formula (A), Formula (A-1), Formula (A-1a), Formula (A-1b), Formula (A-2a), Formula (A-2b), Formula (A-2c), and Formula (A-2d), L¹ is selected from the group consisting of:

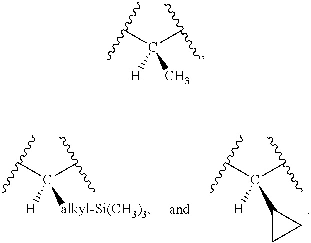

In one embodiment, in each of Formula (A), Formula (A-1), Formula (A-1a), Formula (A-1b), Formula (A-2a), Formula (A-2b), Formula (A-2c), and Formula (A-2d), L¹ is selected from the group consisting of:

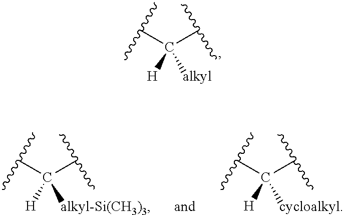

In one embodiment, in each of Formula (A), Formula (A-1), Formula (A-1a), Formula (A-1 b), Formula (A-2a), Formula (A-2b), Formula (A-2c), and Formula (A-2d), L¹ is selected from the group consisting of:

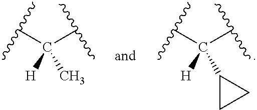

In one embodiment, in each of Formula (A), Formula (A-1), Formula (A-1a), Formula (A-1b), Formula (A-2a), Formula (A-2b), Formula (A-2c), and Formula (A-2d), L¹ is selected from the group consisting of:

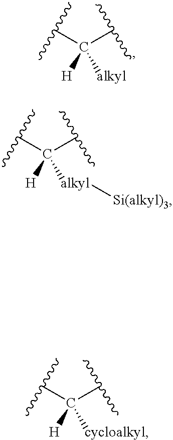

### and —(CH₂)₁₋₃—.

In one embodiment, in each of Formula (A), Formula (A-1), Formula (A-1a), Formula (A-1b), Formula (A-2a), Formula (A-2b), Formula (A-2c), and Formula (A-2d), L¹ is selected from the group consisting of

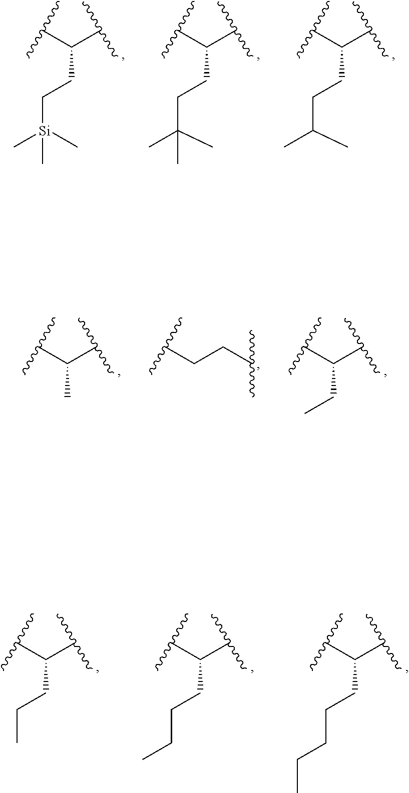

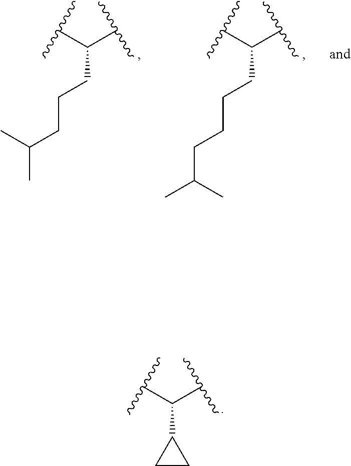

In one embodiment, in each of Formula (A), Formula (A-1), Formula (A-1a), Formula (A-1b), Formula (A-2a), Formula (A-2b), Formula (A-2c), and Formula (A-2d), L¹ is selected from the group consisting of

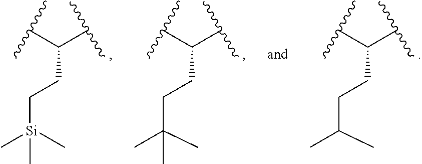

In one embodiment, in each of Formula (A), Formula (A-1), Formula (A-1a), Formula (A-1b), Formula (A-2a), Formula (A-2b), Formula (A-2c), and Formula (A-2d), L¹ is selected from the group consisting of

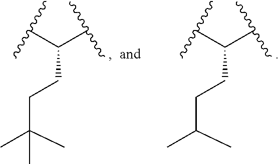

In embodiments wherein L¹ contains a group —(C(R^{5A})₂)—, any two R^{5A} groups bound to the same carbon atom may be taken together to form a carbonyl group, an oxime group, or a substituted oxime group. As indicated herein, each R^{5A} group is selected independently. Similarly, in embodiments wherein L¹ contains a group —(C(R⁵)₂)—, any two R⁵ groups bound to the same carbon atom may be taken together to form a carbonyl group, or an oxime group, wherein the oxygen substituent of each said oxime is independently selected from R¹⁵. For illustrative purposes only, such oxime groups, when present, may be pictured as:

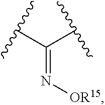

wherein each wavy line presents a point of attachment to the rest of the molecule and wherein R¹⁵ is as described above.

In one embodiment, in each of Formula (A), Formula (A-1), Formula (A-1a), Formula (A-1b), Formula (A-2a), Formula (A-2b), Formula (A-2c), and Formula (A-2d), G is selected from the group consisting of: hydrogen, —NH₂, —OH, halo, cyano, —CHO, cycloalkyl, —N(R¹)-cycloalkyl, heterocycloalkyl, —N(R¹)-heterocycloalkyl, cycloalkenyl, —N(R¹)-cycloalkenyl, heterocycloalkenyl, —N(R¹)-heterocycloalkenyl, alkyl, —N(R¹)-alkyl, heteroalkyl, —N(R¹)-heteroalkyl, alkenyl, —N(R¹)-alkenyl, alkynyl, —N(R¹)-alkynyl,

wherein said heteroalkyl, said heterocycloalkyl, and said heterocycloalkenyl of G may be connected through any available carbon or heteroatom,

and wherein said cycloalkyl said heterocycloalkyl, said alkenyl, said alkynyl, said cycloalkenyl, and said heterocycloalkenyl of G are unsubstituted or substituted with one or more groups independently selected from (1a), (2a), (3a), (4a), (5a), (6a), (7a), (8a), (10a), (12a) and (13a) above;

and wherein said alkyl and said heteroalkyl of G are unsubstituted or substituted with one or more groups independently selected from (1f), (2f), (3f), (4f), (51), (6f), (70, (81), and (10f) above; and wherein R¹ is independently selected from: hydrogen, cycloalkyl, heterocycloalkyl, cycloalkenyl, heterocycloalkenyl, alkyl, heteroalkyl, alkenyl, and alkynyl;

wherein said heteroalkyl, said heterocycloalkyl, and said heterocycloalkenyl of R¹ may be connected through any available carbon or heteroatom,

and wherein said cycloalkyl said heterocycloalkyl, said alkenyl, said alkynyl, said cycloalkenyl, and said heterocycloalkenyl of R¹ are unsubstituted or substituted with one or more groups independently selected from (1a), (2a), (3a), (4a), (5a), (6a), (7a), (8a), (10a), (12a) and (13a) above,

and wherein said alkyl and said heteroalkyl of R¹ are unsubstituted or substituted with one or more groups independently selected from (10, (21), (3f), (4f), (5f), (6f), (71), (8f), and (10f) above:

In one embodiment, in each of Formula (A), Formula (A-1), Formula (A-1a), Formula (A-1b), Formula (A-2a), Formula (A-2b), Formula (A-2c), and Formula (A-2d), G is selected from the group consisting of: hydrogen, —NH₂, —OH, halo, cyano, —CHO, cycloalkyl, —N(R¹)-cycloalkyl, heterocycloalkyl, —N(R¹)-heterocycloalkyl, cycloalkenyl, —N(R¹)-cycloalkenyl, heterocycloalkenyl, —N(R¹)-heterocycloalkenyl, alkyl, —N(R¹)-alkyl, heteroalkyl, —N(R¹)-heteroalkyl, alkenyl, —N(R¹)-alkenyl, alkynyl, —N(R¹)-alkynyl,
wherein said heteroalkyl, said heterocycloalkyl, and said heterocycloalkenyl of G may be connected through any available carbon or heteroatom,
and wherein said cycloalkyl said heterocycloalkyl, said alkenyl, said alkynyl, said cycloalkenyl, and said heterocycloalkenyl of G are unsubstituted or substituted with one or more groups independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl, aryl, —O-aryl, —C(O)-aryl, heteroaryl, —O-heteroaryl, —C(O)-heteroaryl,
wherein each of said alkyl, said alkenyl, said alkynyl, said cycloalkyl, said cycloalkenyl, said aryl, said heteroalkyl, said heterocycloalkyl, said heterocycloalkenyl, and said heteroaryl are unsubstituted or optionally independently substituted with one or more groups each independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl, aryl, —O-aryl, —C(O)-aryl, heteroaryl, —O-heteroaryl, —C(O)-heteroaryl,
and wherein said alkyl and said heteroalkyl of G are unsubstituted or substituted with one or more groups independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl;
wherein each of said alkyl, said alkenyl, said alkynyl, said cycloalkyl, said cycloalkenyl, said heteroalkyl, said heterocycloalkyl and said heterocycloalkenyl are unsubstituted or optionally independently substituted with one or more groups each independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl,
   and wherein R¹ is independently selected from: hydrogen, cycloalkyl, heterocycloalkyl, cycloalkenyl, heterocycloalkenyl, alkyl, heteroalkyl, alkenyl, alkynyl;

wherein said heteroalkyl, said heterocycloalkyl, and said heterocycloalkenyl of R¹ may be connected through any available carbon or heteroatom,

and wherein said cycloalkyl said heterocycloalkyl, said alkenyl, said alkynyl, said cycloalkenyl, and said heterocycloalkenyl of R¹ are unsubstituted or substituted with one or more groups independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl, aryl, —O-aryl, —C(O)-aryl, heteroaryl, —O-heteroaryl, —C(O)-heteroaryl,
wherein each of said alkyl, said alkenyl, said alkynyl, said cycloalkyl, said cycloalkenyl, said aryl, said heteroalkyl, said heterocycloalkyl, said heterocycloalkenyl, and said heteroaryl are unsubstituted or optionally independently substituted with one or more groups each independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl, aryl, —O-aryl, —C(O)-aryl, heteroaryl, —O-heteroaryl, —C(O)-heteroaryl,
and wherein said alkyl, said heteroalkyl of R¹ are unsubstituted or substituted with one or more groups independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl;
wherein each of said alkyl, said alkenyl, said alkynyl, said cycloalkyl, said cycloalkenyl, said heteroalkyl, said heterocycloalkyl and said heterocycloalkenyl are unsubstituted or optionally independently substituted with one or more groups each independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl.

In one embodiment, in each of Formula (A), Formula (A-1), Formula (A-1a), Formula (A-1b), Formula (A-2a), Formula (A-2b), Formula (A-2c), and Formula (A-2d), G is selected from the group consisting of: hydrogen, cycloalkyl, —N(R¹)cycloalkyl, heterocycloalkyl, alkyl, —N(R¹)-alkyl, heteroalkyl, —N(R¹)-heteroalkyl, and alkenyl,
wherein said heterocycloalkyl and said heteroalkyl of G may be connected through any available carbon or heteroatom,
and wherein said cycloalkyl, said alkenyl and said heterocycloalkyl of G are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
wherein each of said alkyl, said cycloalkyl, and said aryl are unsubstituted or optionally independently substituted with one or more groups each independently selected from: halo, cyano, cycloalkyl, alkyl, —O-alkyl,
and wherein said alkyl and said heteroalkyl of G is unsubstituted or substituted with one or more groups independently selected from: halo, cyano, cycloalkyl, —O-alkyl,
wherein each of said alkyl and said cycloalkyl are unsubstituted or optionally independently substituted with one or more groups each independently selected from: halo, cyano, cycloalkyl, —O-cycloalkyl, alkyl, —O-alkyl
   and wherein R¹ is independently selected from: hydrogen, cycloalkyl, heterocycloalkyl, alkyl, heteroalkyl,

wherein said heteroalkyl and said heterocycloalkyl of R¹ may be connected through any available carbon or heteroatom,

and wherein said cycloalkyl and said heterocycloalkyl of R¹ are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
wherein each of said alkyl, said cycloalkyl, and said aryl are unsubstituted or optionally independently substituted with one or more groups each independently selected from: halo, cyano, cycloalkyl, alkyl, —O-alkyl,
and wherein said alkyl and said heteroalkyl of R¹ are unsubstituted or substituted with one or more groups independently selected from: halo, cyano, cycloalkyl, alkyl,
wherein each of said alkyl and said cycloalkyl are unsubstituted or optionally independently substituted with one or more groups each independently selected from: halo, cyano, cycloalkyl, —O-cycloalkyl, alkyl, —O-alkyl.

In one embodiment, in each of Formula (A), Formula (A-1), Formula (A-1a), Formula (A-1b), Formula (A-2a), Formula (A-2b), Formula (A-2c), and Formula (A-2d), G is selected from morpholinyl,
wherein said morpholinyl may be connected through any available carbon or heteroatom, and wherein said morpholinyl is unsubstituted or substituted with one or more groups independently selected from (1a), (2a), (3a), (4a), (5a), (6a), (7a), (8a), (10a), (12a) and (13a) above.

In one embodiment, in each of Formula (A), Formula (A-1), Formula (A-1a), Formula (A-1 b), Formula (A-2a), Formula (A-2b), Formula (A-2c), and Formula (A-2d), G is selected from morpholinyl,
wherein said morpholinyl may be connected through any available carbon or heteroatom, and wherein said morpholinyl is unsubstituted or substituted with one or more groups independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl, aryl, —O-aryl, —C(O)-aryl, heteroaryl, —O-heteroaryl, —C(O)-heteroaryl,
   wherein each of said alkyl, said alkenyl, said alkynyl, said cycloalkyl, said cycloalkenyl, said aryl, said heteroalkyl, said heterocycloalkyl, said heterocycloalkenyl, and said heteroaryl are unsubstituted or optionally independently substituted with one or more groups each independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl, aryl, —O-aryl, —C(O)-aryl, heteroaryl, —O-heteroaryl, and —C(O)-heteroaryl.

In one embodiment, in each of Formula (A), Formula (A-1), Formula (A-1a), Formula (A-1b), Formula (A-2a), Formula (A-2b), Formula (A-2c), and Formula (A-2d), G is selected from morpholinyl,
wherein said morpholinyl may be connected through any available carbon or heteroatom, and wherein said morpholinyl is unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
   wherein each of said alkyl, said cycloalkyl, and said aryl are unsubstituted or optionally independently substituted with one or more groups each independently selected from: halo, cyano, cycloalkyl, alkyl, and —O-alkyl.

In one embodiment, in each of Formula (A), Formula (A-1), Formula (A-1a), Formula (A-1b), Formula (A-2a), Formula (A-2b), Formula (A-2c), and Formula (A-2d), G is selected from piperidinyl,
wherein said piperidinyl may be connected through any available carbon or heteroatom, and wherein said piperidinyl is unsubstituted or substituted with one or more groups independently selected from (1a), (2a), (3a), (4a), (5a), (6a), (7a), (8a), (10a), (12a) and (13a) above.

In one embodiment, in each of Formula (A), Formula (A-1), Formula (A-1a), Formula (A-1b), Formula (A-2a), Formula (A-2b), Formula (A-2c), and Formula (A-2d), G is selected from piperidinyl,
wherein said piperidinyl may be connected through any available carbon or heteroatom, and wherein said piperidinyl is unsubstituted or substituted with one or more groups independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —O(O)-alkynyl, aryl, —O-aryl, —C(O)-aryl, heteroaryl, —O-heteroaryl, —C(O)-heteroaryl,
   wherein each of said alkyl, said alkenyl, said alkynyl, said cycloalkyl, said cycloalkenyl, said aryl, said heteroalkyl, said heterocycloalkyl, said heterocycloalkenyl, and said heteroaryl are unsubstituted or optionally independently substituted with one or more groups each independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl, aryl, —O-aryl, —C(O)-aryl, heteroaryl, —O-heteroaryl, and —C(O)-heteroaryl.

In one embodiment, in each of Formula (A), Formula (A-1), Formula (A-1a), Formula (A-1b), Formula (A-2a), Formula (A-2b), Formula (A-2c), and Formula (A-2d), G is selected from piperidinyl,
wherein said piperidinyl is connected to the core moiety through the ring nitrogen, and wherein said piperidinyl is unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
   wherein each of said alkyl, said cycloalkyl, and said aryl are unsubstituted or optionally independently substituted with one or more groups each independently selected from: halo, cyano, cycloalkyl, alkyl, and —O-alkyl.

In one embodiment, in each of Formula (A), Formula (A-1), Formula (A-1a), Formula (A-1b), Formula (A-2a), Formula (A-2b), and Formula (A-2d), each R² is independently selected from the group consisting of aryl, wherein said aryl of R² are unsubstituted or substituted with one or more groups independently selected from: halo, —Si(R⁷)₃, —SF₅, —CHO, cycloalkyl, —O-cycloalkyl, —C(O)-cycloalkyl, —CO₂-cycloalkyl, —S(O)-cycloalkyl, —S(O)₂-cycloalkyl, —C(O)—N(R²⁰)-cycloalkyl, —S(O)—N(R²⁰)-cycloalkyl, —S(O)₂—N(R²⁰)-cycloalkyl, —C(O)-heterocycloalkyl, —S(O)₂-heterocycloalkyl, cycloalkenyl, —O-cycloalkenyl, —C(O)-cycloalkenyl, —CO₂-cycloalkenyl, —S(O)-cycloalkenyl, —S(O)₂-cycloalkenyl, —C(O)—N(R²⁰)-cycloalkenyl, —S(O)—N(R²⁰)-cycloalkenyl, —S(O)₂—N(R²⁰)-cycloalkenyl, —C(O)-heterocycloalkenyl, —S(O)₂-heterocycloalkenyl, alkyl, —O-alkyl, —C(O)-alkyl, —CO₂-alkyl, —S(O)-alkyl, —S(O)₂-alkyl, —C(O)—N(R²⁰)-alkyl, —S(O)—N(R²⁰)-alkyl, —S(O)₂—N(R²⁰)-alkyl, —C(O)-heteroalkyl, —S(O)₂-heteroalkyl, alkenyl, —O-alkenyl, —C(O)-alkenyl, —CO₂-alkenyl, —S(O)-alkenyl, —S(O)₂-alkenyl, —C(O)—N(R²⁰)-alkenyl, —S(O)—N(R²⁰)-alkenyl, —S(O)₂—N(R²⁰)-alkenyl, alkynyl, —O-alkynyl, —C(O)-alkynyl, —S(O)-alkynyl, —S(O)₂-alkynyl, —C(O)—N(R²⁰)-alkynyl, —S(O)—N(R²⁰)-alkynyl, —S(O)₂—N(R²⁰)-alkynyl, aryl, —O-aryl, —C(O)-aryl, —CO₂-aryl, —S(O)-aryl, —S(O)₂-aryl, —C(O)—N(R²⁰) aryl)-S(O)—N(R²⁰)-aryl, —S(O)₂—N(R²⁰)-aryl,
wherein said heteroalkyl, said heterocycloalkyl, and said heterocycloalkenyl may be connected through any available carbon or heteroatom,
and wherein each of said alkyl, said alkenyl, said alkynyl, said cycloalkyl, said cycloalkenyl, said aryl, said heteroalkyl, said heterocycloalkyl, and said heterocycloalkenyl are unsubstituted or optionally independently substituted with one or more groups each independently selected from: —OH, halo, —Si(R⁷)₃, —CHO, cycloalkyl, cycloalkenyl, alkyl, alkenyl, alkynyl, and aryl.

In one embodiment, in each of Formula (A), Formula (A-1), Formula (A-1a), Formula (A-1b), Formula (A-2a), Formula (A-2b), and Formula (A-2d), ring A represents a spirocycloalkyl ring or a spirocycloalkenyl ring, wherein said ring A is substituted on one or more available ring carbon atoms with from 1 to 5 independently selected R² groups.

In one embodiment, in each of Formula (A), Formula (A-1), Formula (A-1a), Formula (A-1b), Formula (A-2a), Formula (A-2b), and Formula (A-2d), ring A represents a spirocycloalkyl ring, wherein said ring A is substituted on one or more available ring carbon atoms with from 1 to 5 independently selected R² groups.

In one embodiment, in each of Formula (A), Formula (A-1), Formula (A-1a), Formula (A-1b), Formula (A-2a), Formula (A-2b), and Formula (A-2d), each R² is independently selected from the group consisting of: halo, —Si(R⁷), —CHO, cycloalkyl, —O-cycloalkyl, cycloalkenyl, —O-cycloalkenyl, alkyl, —O-alkyl, alkenyl, —O-alkenyl, alkynyl, aryl, —O-aryl,

wherein said alkyl, said cycloalkyl, said alkenyl, said cycloalkenyl, said aryl, and said alkynyl of R² are unsubstituted or substituted with one or more groups independently selected from: halo, —Si(R⁷)₃, —SF₅, —CHO, cycloalkyl, —O-cycloalkyl, —C(O)-cycloalkyl, —CO₂-cycloalkyl, —S(O)-cycloalkyl, —S(O)₂-cycloalkyl, —C(O)—N(R²⁰)-cycloalkyl, —S(O)—N(R²⁰)-cycloalkyl, —S(O)₂—N(R²⁰)-cycloalkyl, —C(O)-heterocycloalkyl, —S(O)₂-heterocycloalkyl, cycloalkenyl, —O-cycloalkenyl, —C(O)-cycloalkenyl, —CO₂-cycloalkenyl, —S(O)-cycloalkenyl, —S(O)₂-cycloalkenyl, —C(O)—N(R²⁰)-cycloalkenyl, —S(O)—N(R²⁰)-cycloalkenyl, —S(O)₂—N(R²⁰)-cycloalkenyl, —C(O)-heterocycloalkenyl, —S(O)₂-heterocycloalkenyl, alkyl, —O-alkyl, —C(O)-alkyl, —CO₂-alkyl, —S(O)-alkyl, —S(O)₂-alkyl, —C(O)—N(R²⁰)-alkyl, —S(O)—N(R²⁰)-alkyl, —S(O)₂—N(R²⁰)-alkyl, —C(O)-heteroalkyl, —S(O)₂-heteroalkyl, alkenyl, —O-alkenyl, —C(O)-alkenyl, —CO₂-alkenyl, —S(O)-alkenyl, —S(O)₂-alkenyl, —C(O)—N(R²⁰)-alkenyl, —S(O)—N(R²⁰)-alkenyl, —S(O)₂—N(R²⁰)-alkenyl, alkynyl, —O-alkynyl, —C(O)-alkynyl, —S(O)-alkynyl, —S(O)₂-alkynyl, —C(O)—N(R²⁰)-alkynyl, —S(O)—N(R²⁰)-alkynyl, —S(O)₂—N(R²⁰)-alkynyl, aryl, —O-aryl, —C(O)-aryl, —CO₂-aryl, —S(O)-aryl, —S(O)₂-aryl, —C(O)—N(R²⁰)-aryl, —S(O)—N(R²⁰)-aryl, —S(O)₂—N(R²⁰)-aryl;
wherein said heteroalkyl, said heterocycloalkyl, and said heterocycloalkenyl may be connected through any available carbon or heteroatom,
and wherein each of said alkyl, said alkenyl, said alkynyl, said cycloalkyl, said cycloalkenyl, said aryl, said heteroalkyl, said heterocycloalkyl, and said heterocycloalkenyl are unsubstituted or optionally independently substituted with one or more groups each independently selected from: —OH, halo, —Si(R⁷)₃, —CHO, cycloalkyl, cycloalkenyl, alkyl, alkenyl, alkynyl, aryl.

In one embodiment, in each of Formula (A), Formula (A-1), Formula (A-1a), Formula (A-1b), Formula (A-2a), Formula (A-2b), and Formula (A-2d), each R² is independently selected from the group consisting of: unsubstituted phenyl.

In one embodiment, in each of Formula (A), Formula (A-1), Formula (A-1a), Formula (A-1b), Formula (A-2a), Formula (A-2b), and Formula (A-2d), each R² is independently selected from the group consisting of phenyl substituted with from 1 to 5 groups independently selected from halo.

In one embodiment, in each of Formula (A), Formula (A-1), Formula (A-1a), Formula (A-1b), Formula (A-2a), Formula (A-2b), and Formula (A-2d), each R² is independently selected from the group consisting of: halo, —Si(R⁷), cycloalkyl, alkyl;
wherein said alkyl and said cycloalkyl of R² are unsubstituted or substituted with one or more groups independently selected from: halo, —Si(R⁷)₃, —CHO, cycloalkyl, alkyl,
   wherein each of said alkyl and cycloalkyl are unsubstituted or optionally independently substituted with one or more groups each independently selected from: halo, —Si(R⁷)₃, —CHO, alkyl.

In one embodiment, in each of Formula (A), Formula (A-1), Formula (A-1a), Formula (A-1b), Formula (A-2a), Formula (A-2b), and Formula (A-2d), each R² is selected from the group consisting of methyl, ethyl, n-propyl, isopropyl, n-butyl, t-butyl, n-pentyl, t-pentyl and —Si(CH₃)₃.

In one embodiment, in each of Formula (A), Formula (A-1), Formula (A-1a), Formula (A-1 b), Formula (A-2a), Formula (A-2b), and Formula (A-2d), each R² is selected from the group consisting of isopropyl and t-butyl.

In one embodiment, in each of Formula (A), Formula (A-1), Formula (A-1a), Formula (A-1b), Formula (A-2a), Formula (A-2b), and Formula (A-2d), each R² is deuteroalkyl.

In one embodiment, in each of Formula (A), Formula (A-1), Formula (A-1a), Formula (A-1b), Formula (A-2a), Formula (A-2b), and Formula (A-2d), each R² is —C(CD₃)₃,

In one embodiment, in each of Formula (A), Formula (A-1), Formula (A-1a), Formula (A-1 b), Formula (A-2a), Formula (A-2b), and Formula (A-2d), each R² is cycloalkyl, wherein said cycloalkyl of R² are unsubstituted or substituted with one or more groups independently selected from: halo, —Si(R⁷)₃, —SF₅, —CHO, cycloalkyl, —O-cycloalkyl, —C(O)-cycloalkyl, —CO₂-cycloalkyl, —S(O)-cycloalkyl, —S(O)₂-cycloalkyl, —C(O)—N(R²⁰)-cycloalkyl, —S(O)—N(R²⁰)-cycloalkyl, —S(O)₂—N(R²⁰)-cycloalkyl, —C(O)-heterocycloalkyl, —S(O)₂-heterocycloalkyl, cycloalkenyl, —O-cycloalkenyl, —C(O)-cycloalkenyl, —CO₂-cycloalkenyl, —S(O)-cycloalkenyl, —S(O)₂-cycloalkenyl, —C(O)—N(R²⁰)-cycloalkenyl, —S(O)—N(R²⁰)-cycloalkenyl, —S(O)₂—N(R²⁰)-cycloalkenyl, —C(O)-heterocycloalkenyl, —S(O)₂-heterocycloalkenyl, alkyl, —O-alkyl, —C(O)-alkyl, —CO₂-alkyl, —S(O)-alkyl, —S(O)₂-alkyl, —C(O)—N(R²⁰)-alkyl, —S(O)—N(R²⁰)-alkyl, —S(O)₂—N(R²⁰)-alkyl, —C(O)-heteroalkyl, —S(O)₂-heteroalkyl, alkenyl, —O-alkenyl, —C(O)-alkenyl, —CO₂-alkenyl, —S(O)-alkenyl, —S(O)₂-alkenyl, —C(O)—N(R²⁰)-alkenyl, —S(O)—N(R²⁰)-alkenyl, —S(O)₂—N(R²⁰)-alkenyl, alkynyl, —O-alkynyl, —C(O)-alkynyl, —S(O)-alkynyl, —S(O)₂-alkynyl, —C(O)—N(R²⁰)-alkynyl, —S(O)—N(R²⁰)-alkynyl, —S(O)₂—N(R²⁰)-alkynyl, aryl, —O-aryl, —C(O)-aryl, —CO₂-aryl, —S(O)-aryl, —S(O)₂-aryl, —C(O)—N(R²⁰)-aryl, —S(O)—N(R²⁰)-aryl, —S(O)₂—N(R²⁰)-aryl,
wherein said heteroalkyl, said heterocycloalkyl, and said heterocycloalkenyl may be connected through any available carbon or heteroatom,
wherein each of said alkyl, said alkenyl, said alkynyl, said cycloalkyl, said cycloalkenyl, said aryl, said heteroalkyl, said heterocycloalkyl, and said heterocycloalkenyl are unsubstituted or optionally independently substituted with one or more groups each independently selected from: —OH, halo, —Si(R⁷)₃, —CHO, cycloalkyl, cycloalkenyl, alkyl, alkenyl, alkynyl, aryl. Non-limiting examples of R² when R² is cycloalkyl include: cyclopropyl, cyclobutyl, cyclopentyl, cyclohexyl, cycloheptyl, and cyclooctyl. Non-limiting illustrations of points of attachment of such substituents include:

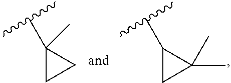

where the wavy line represents the point of attachment of R² to ring A.

In one embodiment, in each of Formula (A), Formula (A-1), Formula (A-1a), Formula (A-1b), Formula (A-2a), Formula (A-2b), and Formula (A-2d), each R² is heterocycloalkyl, wherein said heterocycloalkyl may be connected through any available carbon or heteroatom,

and wherein said heterocycloalkyl of R² is unsubstituted or substituted with one or more groups independently selected from: halo, —Si(R⁷)₃, —SF₅, —CHO, cycloalkyl, —O-cycloalkyl, —C(O)-cycloalkyl, —CO₂-cycloalkyl, —S(O)-cycloalkyl, —S(O)₂-cycloalkyl, —C(O)—N(R²⁰)-cycloalkyl, —S(O)—N(R²⁰)-cycloalkyl, —S(O)₂—N(R²⁰)-cycloalkyl, —C(O)-heterocycloalkyl, —S(O)₂-heterocycloalkyl, cycloalkenyl, —O-cycloalkenyl, —C(O)-cycloalkenyl, —CO₂-cycloalkenyl, —S(O)-cycloalkenyl, —S(O)₂-cycloalkenyl, —C(O)—N(R²⁰)-cycloalkenyl, —S(O)—N(R²⁰)-cycloalkenyl, —S(O)₂—N(R²⁰)-cycloalkenyl, —C(O)-heterocycloalkenyl, —S(O)₂-heterocycloalkenyl, alkyl, —O-alkyl, —C(O)-alkyl, —CO₂-alkyl, —S(O)-alkyl, —S(O)₂-alkyl, —C(O)—N(R²⁰)-alkyl, —S(O)—N(R²⁰)-alkyl, —S(O)₂—N(R²⁰)-alkyl, —C(O)-heteroalkyl, —S(O)₂-heteroalkyl, alkenyl, —O-alkenyl, —C(O)-alkenyl, —CO₂-alkenyl, —S(O)-alkenyl, —S(O)₂-alkenyl, —C(O)—N(R²⁰)-alkenyl, —S(O)—N(R²⁰)-alkenyl, —S(O)₂—N(R²⁰)-alkenyl, alkynyl, —O-alkynyl, —C(O)-alkynyl, —S(O)-alkynyl, —S(O)₂-alkynyl, —C(O)—N(R²⁰)-alkynyl, —S(O)—N(R²⁰)-alkynyl, —S(O)₂—N(R²⁰)-alkynyl, aryl, —O-aryl, —C(O)-aryl, —CO₂-aryl, —S(O)-aryl, —S(O)₂-aryl, —C(O)—N(R²⁰)-aryl, —S(O)—N(R²⁰)-aryl, —S(O)₂—N(R²⁰)-aryl,

wherein said heteroalkyl, said heterocycloalkyl, and said heterocycloalkenyl may be connected through any available carbon or heteroatom,
and wherein each of said alkyl, said alkenyl, said alkynyl, said cycloalkyl, said cycloalkenyl, said aryl, said heteroalkyl, said heterocycloalkyl, and said heterocycloalkenyl are unsubstituted or optionally independently substituted with one or more groups each independently selected from: —OH, halo, —Si(R⁷)₃, —CHO, cycloalkyl, cycloalkenyl, alkyl, alkenyl, alkynyl, aryl. Non-limiting examples of R² when R² is heterocycloalkyl include piperidyl, pyrrolidinyl, piperazinyl, morpholinyl, thiomorpholinyl, thiazolidinyl, 1,4-dioxanyl, tetrahydropyranyl, tetrahydrofuranyl, tetrahydrothiophenyl, lactam, lactone, oxetanes, and the like. Non-limiting illustrations of points of attachment of such substituents when R² is substituted heterocycloalkyl (such as an oxetane or substituted oxetane) include:

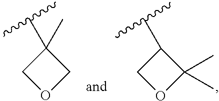

In one embodiment, in each of Formula (A), Formula (A-1), Formula (A-1a), Formula (A-1b), Formula (A-2a), Formula (A-2b), and Formula (A-2d), each R² is —Si(alkyl)₃.

In one embodiment, in each of Formula (A), Formula (A-1), Formula (A-1a), Formula (A-1b), Formula (A-2a), Formula (A-2b), and Formula (A-2d), each R² is —Si(CH₃)₃.

In one embodiment, in each of Formula (A), Formula (A-1), Formula (A-1a), Formula (A-1b), Formula (A-2a), Formula (A-2b), Formula (A-2c), and Formula (A-2d), R³ is H.

In one embodiment, in each of Formula (A), Formula (A-1), Formula (A-1a), Formula (A-1b), Formula (A-2a), Formula (A-2b), Formula (A-2c), and Formula (A-2d), R³ is selected from methyl, ethyl, n-propyl, and isopropyl.

In one embodiment, in each of Formula (A), Formula (A-1), Formula (A-1a), Formula (A-1b), Formula (A-2a), Formula (A-2b), Formula (A-2c), and Formula (A-2d), Z is —(C(R¹¹)₂)—(C(R¹²)(R¹⁵)ₘ—C(O)OH. Pharmaceutically acceptable salts of such acids are also contemplated as being within the scope of the invention. Thus, in another embodiment, in each of Formula (A), Formula (A-1), Formula (A-1a), Formula (A-1 b), Formula (A-2a), Formula (A-2b), Formula (A-2c), and Formula (A-2d), Z is —(C(R¹¹)₂)—(C(R¹²)(R¹³))ₘ—C(O)O⁻Na⁺. Additional non-limiting salts contemplated as alternatives to the sodium salt are known to those of ordinary skill in the art and/or are as described herein.

In one embodiment, in each of Formula (A), Formula (A-1), Formula (A-1a), Formula (A-1b), Formula (A-2a), Formula (A-2b), Formula (A-2c), and Formula (A-2d), Z is —(CH₂)—(CH(CH₃))—C(O)OH.

In one embodiment, in each of Formula (A), Formula (A-1), Formula (A-1a), Formula (A-1b), Formula (A-2a), Formula (A-2b), Formula (A-2c), and Formula (A-2d), Z is —(CH₂)—(CH₂)—(CH₂)—C(O)OH.

In one embodiment, in each of Formula (A), Formula (A-1), Formula (A-1a), Formula (A-1b), Formula (A-2a), Formula (A-2b), Formula (A-2c), and Formula (A-2d), Z is —(CH₂)—C(CH₃)₂—C(O)OH.

In one embodiment, in each of Formula (A), Formula (A-1), Formula (A-1a), Formula (A-1b), Formula (A-2a), Formula (A-2b), Formula (A-2c), and Formula (A-2d), Z is —(CH₂)—C(CH₃)(OH)—C(O)OH.

In one embodiment, in each of Formula (A), Formula (A-1), Formula (A-1a), Formula (A-1b), Formula (A-2a), Formula (A-2b), Formula (A-2c), Formula (A-2d), Z is —CH₂—CH₂-C(O)OH.

In one embodiment, in each of Formula (A), Formula (A-1), Formula (A-1a), Formula (A-1b), Formula (A-2a), Formula (A-2b), Formula (A-2c), Formula (A-2d), Z is —CH₂—CH(OH)—C(O)OH.

In one embodiment, in each of Formula (A), Formula (A-1), Formula (A-1a), Formula (A-1b), Formula (A-2a), Formula (A-2b), Formula (A-2c), and Formula (A-2d), Z is —CH(CH₃)—CH₂—C(O)OH.

In one embodiment, in each of Formula (A), Formula (A-1), Formula (A-1a), Formula (A-1b), Formula (A-2a), Formula (A-2b), Formula (A-2c), and Formula (A-2d), Z is —C(CH₃)₂—CH₂-C(O)OH.

In one embodiment, in each of Formula (A), Formula (A-1), Formula (A-1a), Formula (A-1b), Formula (A-2a), Formula (A-2b), Formula (A-2c), and Formula (A-2d), Z is —(C(R¹¹)₂)—(C(R¹⁴)₂)ₙ—C(O)OH.

In one embodiment, in each of Formula (A), Formula (A-1), Formula (A-1a), Formula (A-1b), Formula (A-2a), Formula (A-2b), Formula (A-2c), and Formula (A-2d), Z is —CH₂—CH(F)-C(O)OH.

In one embodiment, in each of Formula (A), Formula (A-1), Formula (A-1a), Formula (A-1b), Formula (A-2a), Formula (A-2b), Formula (A-2c), and Formula (A-2d), Z is —CH₂—CF₂-C(O)OH.

In one embodiment, in each of Formula (A), Formula (A-1), Formula (A-1a), Formula (A-1 b), Formula (A-2a), Formula (A-2b), Formula (A-2c), and Formula (A-2d), Z is —CH(CH₃)—CF₂—C(O)OH.

In one embodiment, in each of Formula (A), Formula (A-1), Formula (A-1a), Formula (A-1b), Formula (A-2a), Formula (A-2b), Formula (A-2c), and Formula (A-2d), Z is —CH₂—CH₂-CF₂—C(O)OH.

In one embodiment, in each of Formula (A), Formula (A-1), Formula (A-1a), Formula (A-1b), Formula (A-2a), Formula (A-2b), Formula (A-2c), and Formula (A-2d), Z is

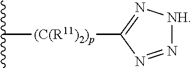

In one embodiment, in each of Formula (A), Formula (A-1), Formula (A-1a), Formula (A-1b), Formula (A-2a), Formula (A-2b), Formula (A-2c), and Formula (A-2d), Z is

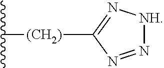

In one embodiment, in each of Formula (A), Formula (A-1), Formula (A-1a), Formula (A-1b), Formula (A-2a), Formula (A-2b), Formula (A-2c), and Formula (A-2d), Z is

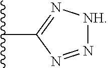 [SMILES: *c1nn[nH]n1]

In one embodiment, in each of Formula (A), Formula (A-1), Formula (A-1a), Formula (A-1b), Formula (A-2a), Formula (A-2b), Formula (A-2c), and Formula (A-2d), when Z is a moiety selected from —(C(R¹¹)₂)—(C(R¹²R¹³))ₘ—C(O)OH, or —(C(R¹¹)₂)—(C(R¹⁴)₂)ₙ—C(O)OH, the —C(O)OH group may be replaced by a moiety -Q, wherein Q is selected from the group consisting of:

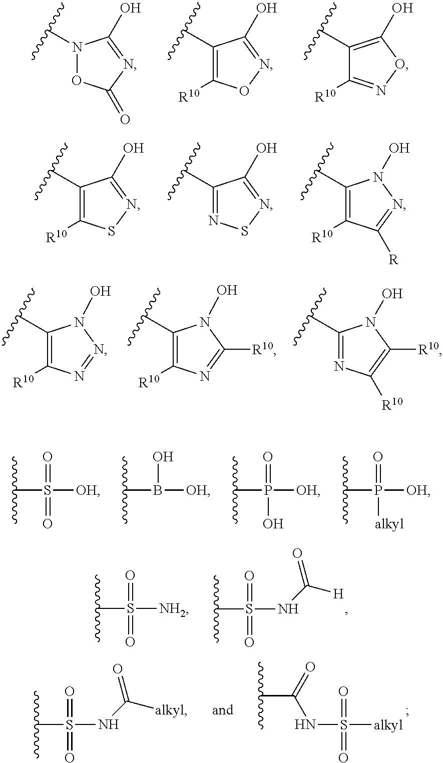

Such moieties Q are readily available to those skilled in the art and may be made, for example, by methods according to Stensbol et at, J. Med. Chem., 2002, 45, 19-31, or according to Moreira Lima et at, Current Med. Chem., 2005, 12, 23-49.

In one embodiment, in Formula (A), the compounds of the invention have the general structure shown in Formula (I):

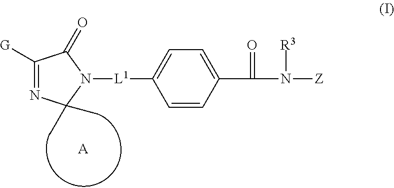

and include pharmaceutically acceptable salts, solvates, esters, prodrugs, tautomers, and isomers of said compounds,

wherein ring A, L¹, G, R³, and Z are selected independently of each other and wherein:

ring A and G are as defined in Formula (A);

L¹ is selected from the group consisting of: a bond, —N(R⁴)—, —N(R⁴)—(C(R^{5A})₂)—, —O—, —O—(C(R^{5A})₂)—, and —(C(R^{5A})₂)—(C(R⁵)₂)ₛ—;

s is 0-3;

R³ is selected from the group consisting of H and lower alkyl;

Z is a moiety selected from —(C(R¹¹)₂)—(C(R¹²R¹³))—C(O)OH, —(C(R¹⁵₂)—(C(R¹⁴)₂)ₙ—C(O)OH, and

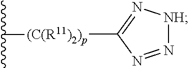

m is an integer from 0 to 5;

n is an integer from 0 to 5;

p is an integer from 0 to 5;

each R⁴ is independently selected from H, lower alkyl, cycloalkyl, heterocycloalkyl, heteroalkyl, and haloalkyl;

each R^{5A} is independently selected from H, lower alkyl, -lower alkyl-Si(CH₃)₃, -lower alkyl-Si(CH₃)₃, lower haloalkyl, and hydroxy-substituted lower alkyl;

each R⁵ is independently selected from H, —OH, lower alkyl, -lower alkyl-Si(CH₃)₃, -lower alkyl-Si(CH₃)₃, lower haloalkyl, and hydroxy-substituted lower alkyl;

each R⁷ is independently selected from H, alkyl, heteroalkyl, and haloalkyl;

each R¹¹ is independently selected from H and lower alkyl;

each R¹² is independently selected from H, lower alkyl, —OH, hydroxy-substituted lower alkyl;

each R¹³ is independently selected from H, unsubstituted lower alkyl, lower alkyl substituted with one or more groups each independently selected from hydroxyl and alkoxy, or R¹² and R¹³ are taken together to form an oxo; and

each R¹⁴ is independently selected from H and fluoro.

In one embodiment, in Formula (I):

ring A represents a spirocycloalkyl ring or a spirocycloalkenyl ring, wherein said ring A is substituted on one or more available ring carbon atoms with from 0 to 5 independently selected R² groups;

G is selected from the group consisting of: hydrogen, —NH₂, —OH, halo, cyano, —CHO, cycloalkyl, —N(R¹)-cycloalkyl, heterocycloalkyl, —N(R¹)-heterocycloalkyl, cycloalkenyl, —N(R¹)-cycloalkenyl, heterocycloalkenyl, —N(R¹)-heterocycloalkenyl, alkyl, —N(R¹)-alkyl, heteroalkyl, —N(R¹)-heteroalkyl, alkenyl, —N(R¹)-alkenyl, alkynyl, —N(R¹)-alkynyl,
wherein said heteroalkyl, said heterocycloalkyl, and said heterocycloalkenyl of G may be connected through any available carbon or heteroatom, and wherein said cycloalkyl said heterocycloalkyl, said alkenyl, said alkynyl, said cycloalkenyl, and said heterocycloalkenyl of G are unsubstituted or substituted with one or more groups independently selected from (1a), (2a), (3a), (4a), (5a), (6a), (7a), (8a), (10a), (12a) and (13a) above, and wherein said alkyl and said heteroalkyl of G are unsubstituted or substituted with one or more groups independently selected from (10, (2f), (3f), (4f), (5f), (6f), (7f), (8f), and (10f) above;
   R¹ is independently selected from: hydrogen, cycloalkyl, heterocycloalkyl, cycloalkenyl, heterocycloalkenyl, alkyl, heteroalkyl, alkenyl, and alkynyl;
wherein said heteroalkyl, said heterocycloalkyl, and said heterocycloalkenyl of R¹ may be connected through any available carbon or heteroatom, and wherein said cycloalkyl said heterocycloalkyl, said alkenyl, said alkynyl, said cycloalkenyl, and said heterocycloalkenyl of R¹ are unsubstituted or substituted with one or more groups independently selected from (1a), (2a), (3a), (4a), (5a), (6a), (7a), (8a), (10a), (12a) and (13a) above, and wherein said alkyl and said heteroalkyl of R¹ are unsubstituted or substituted with one or more groups independently selected from (1f), (2f), (3f), (4f), (5f), (6f), (7f), (8f), and (10f) above;
   In one embodiment, in Formula (I):

ring A represents a spirocycloalkyl ring or a spirocycloalkenyl ring, wherein said ring A is substituted on one or more available ring carbon atoms with from 0 to 5 independently selected R² groups;

G is selected from the group consisting of: hydrogen, —NH₂, —OH, halo, cyano, —CHO, cycloalkyl, —N(R¹)-cycloalkyl, heterocycloalkyl, —N(R¹)-heterocycloalkyl, cycloalkenyl, —N(R¹)-cycloalkenyl, heterocycloalkenyl, —N(R¹)-heterocycloalkenyl, alkyl, —N(R¹)-alkyl, heteroalkyl, —N(R¹)-heteroalkyl, alkenyl, —N(R¹)-alkenyl, alkynyl, —N(R¹)-alkynyl;
wherein said heteroalkyl, said heterocycloalkyl, and said heterocycloalkenyl of G may be connected through any available carbon or heteroatom, and wherein said cycloalkyl said heterocycloalkyl, said alkenyl, said alkynyl, said cycloalkenyl, and said heterocycloalkenyl of G are unsubstituted or substituted with one or more groups independently selected from: halo, —Si(R)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl, aryl, —O-aryl, —C(O)-aryl, heteroaryl, —O-heteroaryl, —C(O)-heteroaryl,
wherein each of said alkyl, said alkenyl, said alkynyl, said cycloalkyl, said cycloalkenyl, said aryl, said heteroalkyl, said heterocycloalkyl, said heterocycloalkenyl, and said heteroaryl are unsubstituted or optionally independently substituted with one or more groups each independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl, aryl, —O-aryl, —C(O)-aryl, heteroaryl, —O-heteroaryl, —C(O)-heteroaryl,

and wherein said alkyl and said heteroalkyl of G are unsubstituted or substituted with one or more groups independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl;
wherein each of said alkyl, said alkenyl, said alkynyl, said cycloalkyl, said cycloalkenyl, said heteroalkyl, said heterocycloalkyl and said heterocycloalkenyl are unsubstituted or optionally independently substituted with one or more groups each independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl;
   R¹ is independently selected from: hydrogen, cycloalkyl, heterocycloalkyl, cycloalkenyl, heterocycloalkenyl, alkyl, heteroalkyl, alkenyl, and alkynyl;
wherein said heteroalkyl, said heterocycloalkyl, and said heterocycloalkenyl of R¹ may be connected through any available carbon or heteroatom, and wherein said cycloalkyl said heterocycloalkyl, said alkenyl, said alkynyl, said cycloalkenyl, and said heterocycloalkenyl of R¹ are unsubstituted or substituted with one or more groups independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl, aryl, —O-aryl, —C(O)-aryl, heteroaryl, —O-heteroaryl, —C(O)-heteroaryl,
wherein each of said alkyl, alkenyl, alkynyl, cycloalkyl, cycloalkenyl, aryl, heteroalkyl, heterocycloalkyl, heterocycloalkenyl, and heteroaryl are unsubstituted or optionally independently substituted with one or more groups each independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl, aryl, —O-aryl, —C(O)-aryl, heteroaryl, —O-heteroaryl, —C(O)-heteroaryl,
and wherein said alkyl and said heteroalkyl of R¹ are unsubstituted or substituted with one or more groups independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl;
wherein each of said alkyl, said alkenyl, said alkynyl, said cycloalkyl, said cycloalkenyl, said heteroalkyl, said heterocycloalkyl and said heterocycloalkenyl are unsubstituted or optionally independently substituted with one or more groups each independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl, and
   each R² (when present) is independently selected from the group consisting of —Si(CH₃)₃ and alkyl, wherein said alkyl is substituted with from 0 to 5 groups independently selected from: halo, —Si(R⁷)₃, —SF₅, —CHO, cycloalkyl, —O-cycloalkyl, —C(O)-cycloalkyl, —CO₂-cycloalkyl, —S(O)-cycloalkyl, —S(O)₂-cycloalkyl, —C(O)—N(R²⁰)-cycloalkyl, —S(O)—N(R²⁰)-cycloalkyl, —S(O)₂—N(R²⁰)-cycloalkyl, —C(O)-heterocycloalkyl, —S(O)₂-heterocycloalkyl, cycloalkenyl, —O-cycloalkenyl, —C(O)-cycloalkenyl, —CO₂-cycloalkenyl, —S(O)-cycloalkenyl, —S(O)₂-cycloalkenyl, —C(O)—N(R²⁰)-cycloalkenyl, —S(O)—N(R²⁰)-cycloalkenyl, —S(O)₂—N(R²⁰)-cycloalkenyl, —C(O)-heterocycloalkenyl, —S(O)₂-heterocycloalkenyl, alkyl, —O-alkyl, —C(O)-alkyl, —CO₂-alkyl, —S(O)-alkyl, —S(O)₂-alkyl, —C(O)—N(R²⁰)-alkyl, —S(O)—N(R²⁰)-alkyl, —S(O)₂—N(R²⁰)-alkyl, —C(O)-heteroalkyl, —S(O)₂-heteroalkyl, alkenyl, —O-alkenyl, —C(O)-alkenyl, —CO₂-alkenyl, —S(O)-alkenyl, —S(O)₂-alkenyl, —C(O)—N(R²⁰)-alkenyl, —S(O)—N(R²⁰)-alkenyl, —S(O)₂—N(R²⁰)-alkenyl, alkynyl, —O-alkynyl, —C(O)-alkynyl, —S(O)-alkynyl, —S(O)₂-alkynyl, —C(O)—N(R²⁰)-alkynyl, —S(O)—N(R²⁰)-alkynyl, —S(O)₂—N(R²⁰)-alkynyl, aryl, —O-aryl, —C(O)-aryl, —CO₂-aryl, —S(O)-aryl, —S(O)₂-aryl, —C(O)—N(R²⁰)-aryl, —S(O)—N(R²⁰)-aryl, —S(O)₂—N(R²⁰)-aryl,
wherein each of said alkyl, said alkenyl, said alkynyl, said cycloalkyl, said cycloalkenyl, said aryl, said heteroalkyl, said heterocycloalkyl, and said heterocycloalkenyl are unsubstituted or optionally independently substituted with one or more groups each independently selected from: —OH, halo, —Si(R⁷)₃, —CHO, cycloalkyl, cycloalkenyl, alkyl, alkenyl, alkynyl, aryl.
   In one embodiment, in Formula (I):

ring A represents a spirocycloalkyl ring or a spirocycloalkenyl ring, wherein said ring A is substituted on one or more available ring carbon atoms with from 0 to 5 independently selected R² groups;

G is selected from the group consisting of: hydrogen, cycloalkyl, —N(R¹)cycloalkyl, heterocycloalkyl, alkyl, —N(R¹)-alkyl, heteroalkyl, —N(R¹)-heteroalkyl, alkenyl,
wherein said heterocycloalkyl and said heteroalkyl of G may be connected through any available carbon or heteroatom, and wherein said cycloalkyl, said alkenyl and said heterocycloalkyl of G are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
   wherein each of said alkyl, said cycloalkyl, and said aryl are unsubstituted or optionally independently substituted with one or more groups each independently selected from: halo, cyano, cycloalkyl, alkyl, —O-alkyl,
and wherein said alkyl and said heteroalkyl of G is unsubstituted or substituted with one or more groups independently selected from: halo, cyano, cycloalkyl, —O-alkyl,
   wherein each of said alkyl and said cycloalkyl are unsubstituted or optionally independently substituted with one or more groups each independently selected from: halo, cyano, cycloalkyl, —O-cycloalkyl, alkyl, —O-alkyl,
      and wherein R¹ is independently selected from: hydrogen, cycloalkyl, heterocycloalkyl, alkyl, heteroalkyl,
wherein said heteroalkyl and said heterocycloalkyl of R¹ may be connected through any available carbon or heteroatom, and wherein said cycloalkyl and said heterocycloalkyl of R¹ are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
   wherein each of said alkyl, said cycloalkyl, and said aryl are unsubstituted or optionally independently substituted with one or more groups each independently selected from: halo, cyano, cycloalkyl, alkyl, —O-alkyl,
and wherein said alkyl and said heteroalkyl of R¹ are unsubstituted or substituted with one or more groups independently selected from: halo, cyano, cycloalkyl, alkyl,
   wherein each of said alkyl and said cycloalkyl are unsubstituted or optionally independently substituted with one or more groups each independently selected from: halo, cyano, cycloalkyl, —O-cycloalkyl, alkyl, —O-alkyl,
      each R² is independently selected from the group consisting of: halo, —Si(R⁷), cycloalkyl, alkyl;
wherein said alkyl and said cycloalkyl of R² are unsubstituted or substituted with one or more groups independently selected from: halo, —Si(R⁷)₃, —CHO, cycloalkyl, alkyl,
   wherein each of said alkyl and cycloalkyl are unsubstituted or optionally independently substituted with one or more groups each independently selected from: halo, —Si(R⁷)₃, —CHO, alkyl.
      In one embodiment, in Formula (I):

ring A represents a spirocycloalkyl ring or a spirocycloalkenyl ring, wherein said ring A is substituted on one or more available ring carbon atoms with from 0 to 5 independently selected R² groups;

G is selected from morpholinyl,
wherein said morpholinyl of G may be connected through any available carbon or heteroatom, and wherein said morpholinyl of G is unsubstituted or substituted with one or more groups independently selected from (1a), (2a), (3a), (4a), (5a), (6a), (7a), (8a), (10a), (12a) and (13a) above.
   In one embodiment, in Formula (I):

ring A represents a spirocycloalkyl ring or a spirocycloalkenyl ring, wherein said ring A is substituted on one or more available ring carbon atoms with from 0 to 5 independently selected R² groups;

G is selected from piperidinyl,
wherein said piperidinyl of G may be connected through any available carbon or heteroatom, and wherein said piperidinyl of G is unsubstituted or substituted with one or more groups independently selected from (1a), (2a), (3a), (4a), (5a), (6a), (7a), (8a), (10a), (12a) and (13a) above.
   In one embodiment, in Formula (I):

ring A represents a spirocycloalkyl ring or a spirocycloalkenyl ring, wherein said ring A is substituted on one or more available ring carbon atoms with from 0 to 5 independently selected R² groups;

G is selected from morpholinyl,
wherein said morpholinyl of G is connected through nitrogen, and wherein said morpholinyl of G is unsubstituted or substituted with one or more groups independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl, aryl, —O-aryl, —C(O)-aryl, heteroaryl, —O-heteroaryl, —C(O)-heteroaryl,
wherein each of said alkyl, said alkenyl, said alkynyl, said cycloalkyl, said cycloalkenyl, said aryl, said heteroalkyl, said heterocycloalkyl, said heterocycloalkenyl, and said heteroaryl are unsubstituted or optionally independently substituted with one or more groups each independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl, aryl, —O-aryl, —C(O)-aryl, heteroaryl, —O-heteroaryl, —C(O)-heteroaryl,
   each R² (when present) is independently selected from the group consisting of —Si(CH₃)₃ and alkyl, wherein said alkyl is substituted with from 0 to 5 groups independently selected from: halo, —Si(R⁷)₃, —SF₅, —CHO, cycloalkyl, —O-cycloalkyl, —C(O)-cycloalkyl, —CO₂-cycloalkyl, —S(O)-cycloalkyl, —S(O)₂-cycloalkyl, —C(O)—N(R²⁰)-cycloalkyl, —S(O)—N(R²⁰)-cycloalkyl, —S(O)₂—N(R²⁰)-cycloalkyl, —C(O)-heterocycloalkyl, —S(O)₂-heterocycloalkyl, cycloalkenyl, —O-cycloalkenyl, —C(O)-cycloalkenyl, —CO₂-cycloalkenyl, —S(O)-cycloalkenyl, —S(O)₂-cycloalkenyl, —C(O)—N(R²⁰)-cycloalkenyl, —S(O)—N(R²⁰)-cycloalkenyl, —S(O)₂—N(R²⁰)-cycloalkenyl, —C(O)-heterocycloalkenyl, —S(O)₂-heterocycloalkenyl, alkyl, —O-alkyl, —C(O)-alkyl, —CO₂-alkyl, —S(O)-alkyl, —S(O)₂-alkyl, —C(O)—N(R²⁰)-alkyl, —S(O)—N(R²⁰)-alkyl, —S(O)₂—N(R²⁰)-alkyl, —C(O)-heteroalkyl, —S(O)₂-heteroalkyl, alkenyl, —O-alkenyl, —C(O)-alkenyl, —CO₂-alkenyl, —S(O)-alkenyl, —S(O)₂-alkenyl, —C(O)—N(R²⁰)-alkenyl, —S(O)—N(R²⁰)-alkenyl, —S(O)₂—N(R²⁰)-alkenyl, alkynyl, —O-alkynyl, —C(O)-alkynyl, —S(O)-alkynyl, —S(O)₂-alkynyl, —C(O)—N(R²⁰)-alkynyl, —S(O)—N(R²⁰)-alkynyl, —S(O)₂—N(R²⁰)-alkynyl, aryl, —O-aryl, —C(O)-aryl, —CO₂-aryl, —S(O)-aryl, —S(O)₂-aryl, —C(O)—N(R²⁰)-aryl, —S(O)—N(R²⁰)-aryl, —S(O)₂—N(R²⁰)-aryl,
wherein each of said alkyl, said alkenyl, said alkynyl, said cycloalkyl, said cycloalkenyl, said aryl, said heteroalkyl, said heterocycloalkyl, and said heterocycloalkenyl are unsubstituted or optionally independently substituted with one or more groups each independently selected from: —OH, halo, —Si(R⁷)₃, —CHO, cycloalkyl, cycloalkenyl, alkyl, alkenyl, alkynyl, and aryl.
   In one embodiment, in Formula (I):

ring A represents a spirocycloalkyl ring or a spirocycloalkenyl ring, wherein said ring A is substituted on one or more available ring carbon atoms with from 0 to 5 independently selected R² groups;

G is selected from piperidinyl,
wherein said piperidinyl of G is connected through nitrogen, and wherein said piperidinyl of G is unsubstituted or substituted with one or more groups independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl, aryl, —O-aryl, —C(O)-aryl, heteroaryl, —O-heteroaryl, —C(O)-heteroaryl,
wherein each of said alkyl, said alkenyl, said alkynyl, said cycloalkyl, said cycloalkenyl, said aryl, said heteroalkyl, said heterocycloalkyl, said heterocycloalkenyl, and said heteroaryl are unsubstituted or optionally independently substituted with one or more groups each independently selected from: halo, —Si(R⁷)₃, —SF₅, cyano, —CHO, cycloalkyl, —O-cycloalkyl, —C(O)-cycloalkyl, heterocycloalkyl, —O-heterocycloalkyl, —C(O)-heterocycloalkyl, cycloalkenyl, —O-cycloalkenyl, —C(O)-cycloalkenyl, heterocycloalkenyl, —O-heterocycloalkenyl, —C(O)-heterocycloalkenyl, alkyl, —O-alkyl, —C(O)-alkyl, heteroalkyl, —O-heteroalkyl, —C(O)-heteroalkyl, alkenyl, —O-alkenyl, —C(O)-alkenyl, alkynyl, —O-alkynyl, —C(O)-alkynyl, aryl, —O-aryl, —C(O)-aryl, heteroaryl, —O-heteroaryl, —C(O)-heteroaryl,
   each R² (when present) is independently selected from the group consisting of —Si(CH₃)₃ and alkyl, wherein said alkyl is substituted with from 0 to 5 groups independently selected from: halo, —Si(R⁷)₃, —SF₅, —CHO, cycloalkyl, —O-cycloalkyl, —C(O)-cycloalkyl, —CO₂-cycloalkyl, —S(O)-cycloalkyl, —S(O)₂-cycloalkyl, —C(O)—N(R²⁰)-cycloalkyl, —S(O)—N(R²⁰)-cycloalkyl, —S(O)₂—N(R²⁰)-cycloalkyl, —C(O)-heterocycloalkyl, —S(O)₂-heterocycloalkyl, cycloalkenyl, —O-cycloalkenyl, —C(O)-cycloalkenyl, —CO₂-cycloalkenyl, —S(O)-cycloalkenyl, —S(O)₂-cycloalkenyl, —C(O)—N(R²⁰)-cycloalkenyl, —8(O)—N(R²⁰)-cycloalkenyl, —S(O)₂—N(R²⁰)-cycloalkenyl, —C(O)-heterocycloalkenyl, —S(O)₂-heterocycloalkenyl, alkyl, —O-alkyl, —C(O)-alkyl, —CO₂-alkyl, —S(O)-alkyl, —S(O)₂-alkyl, —C(O)—N(R²⁰)-alkyl, —S(O)—N(R²⁰)-alkyl, —S(O)₂—N(R²⁰)-alkyl, —C(O)-heteroalkyl, —S(O)₂-heteroalkyl, alkenyl, —O-alkenyl, —C(O)-alkenyl, —CO₂-alkenyl, —S(O)-alkenyl, —S(O)₂-alkenyl, —C(O)—N(R²⁰)-alkenyl, —S(O)—N(R²⁰)-alkenyl, —S(O)₂—N(R²⁰)-alkenyl, alkynyl, —O-alkynyl, —C(O)-alkynyl, —S(O)-alkynyl, —S(O)₂-alkynyl, —C(O)—N(R²⁰)-alkynyl, —S(O)—N(R²⁰)-alkynyl, —S(O)₂—N(R²⁰)-alkynyl, aryl, —O-aryl, —C(O)-aryl, —CO₂-aryl, —S(O)-aryl, —S(O)₂-aryl, —C(O)—N(R²⁰)-aryl, —S(O)—N(R²⁰)-aryl, —S(O)₂—N(R²⁰)-aryl,
wherein each of said alkyl, said alkenyl, said alkynyl, said cycloalkyl, said cycloalkenyl, said aryl, said heteroalkyl, said heterocycloalkyl, and said heterocycloalkenyl are unsubstituted or optionally independently substituted with one or more groups each independently selected from: —OH, halo, —Si(R⁷)₃, —CHO, cycloalkyl, cycloalkenyl, alkyl, alkenyl, alkynyl, and aryl.
   In one embodiment, in Formula (I):

ring A represents a spirocycloalkyl ring or a spirocycloalkenyl ring, wherein said ring A is substituted on one or more available ring carbon atoms with from 0 to 5 independently selected R² groups;

G is selected from morpholinyl,
wherein said morpholinyl of G is connected through nitrogen, and wherein said morpholinyl of G is unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
   wherein each of said alkyl, said cycloalkyl, and said aryl are unsubstituted or optionally independently substituted with one or more groups each independently selected from: halo, cyano, cycloalkyl, alkyl, —O-alkyl;
      each R² is independently selected from the group consisting of: halo, —Si(R⁷), cycloalkyl, alkyl;
wherein said alkyl and said cycloalkyl of R² are unsubstituted or substituted with one or more groups independently selected from: halo, —Si(R⁷)₃, —CHO, cycloalkyl, alkyl,
   wherein each of said alkyl and cycloalkyl are unsubstituted or optionally independently substituted with one or more groups each independently selected from: halo, —Si(R⁷)₃, —CHO, alkyl.
      In one embodiment, in Formula (I):

ring A represents a spirocycloalkyl ring or a spirocycloalkenyl ring, wherein said ring A is substituted on one or more available ring carbon atoms with from 0 to 5 independently selected R² groups;

G is selected from piperidinyl,
wherein said piperidinyl of G is connected through nitrogen, and wherein said piperidinyl of G is unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
   wherein each of said alkyl, said cycloalkyl, and said aryl are unsubstituted or optionally independently substituted with one or more groups each independently selected from: halo, cyano, cycloalkyl, alkyl, —O-alkyl;
      each R² is independently selected from the group consisting of: halo, —Si(R¹), cycloalkyl, alkyl;
wherein said alkyl and said cycloalkyl of R² are unsubstituted or substituted with one or more groups independently selected from: halo, —Si(R⁷)₃, —CHO, cycloalkyl, alkyl,
wherein each of said alkyl and cycloalkyl are unsubstituted or optionally independently substituted with one or more groups each independently selected from: halo, —Si(R⁷)₃, —CHO, alkyl.

In one embodiment, the compounds of the invention have the general structure shown in Formula (I-1):

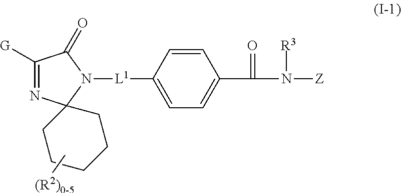

and include pharmaceutically acceptable salts, solvates, esters, prodrugs, tautomers, and isomers of said compounds,

wherein L¹, G, each R², R³, and Z are selected independently of each other and as defined in Formula (I).

In one embodiment, the compounds of the invention have the general structure shown in Formula (ID:

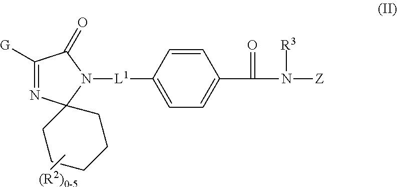

and include pharmaceutically acceptable salts, solvates, esters, prodrugs, tautomers, and isomers of said compounds,

wherein L¹, G, each R², R³, and Z are selected independently of each other and wherein:

L¹ is selected from the group consisting of: a bond and —(C(R^{5A})₂)—(C(R⁵)₂)ₛ—;

s is 0-1;

u is 0 to 2;

v is 1-2;

G is selected from the group consisting of: hydrogen, cycloalkyl, —N(R¹)cycloalkyl, heterocycloalkyl, alkyl, —N(R¹)-alkyl, heteroalkyl, —N(R¹)-heteroalkyl, and alkenyl,
wherein said heterocycloalkyl and said heteroalkyl of G may be connected through any available carbon or heteroatom, and wherein said cycloalkyl, said alkenyl and said heterocycloalkyl of G are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
wherein each of said alkyl, said cycloalkyl, and said aryl are unsubstituted or optionally independently substituted with one or more groups each independently selected from: halo, cyano, cycloalkyl, alkyl, and —O-alkyl,
and wherein said alkyl and said heteroalkyl of G is unsubstituted or substituted with one or more groups independently selected from: halo, cyano, cycloalkyl, —O-alkyl,
wherein each of said alkyl and said cycloalkyl are unsubstituted or optionally independently substituted with one or more groups each independently selected from: halo, cyano, cycloalkyl, —O-cycloalkyl, alkyl, —O-alkyl,

and wherein R¹ is independently selected from: hydrogen, cycloalkyl, heterocycloalkyl, alkyl, heteroalkyl,
wherein said heteroalkyl and said heterocycloalkyl of R¹ may be connected through any available carbon or heteroatom, and wherein said cycloalkyl and said heterocycloalkyl of R¹ are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
wherein each of said alkyl, said cycloalkyl, and said aryl are unsubstituted or optionally independently substituted with one or more groups each independently selected from: halo, cyano, cycloalkyl, alkyl, —O-alkyl,
and wherein said alkyl and said heteroalkyl of R¹ are unsubstituted or substituted with one or more groups independently selected from: halo, cyano, cycloalkyl, alkyl,
wherein each of said alkyl and said cycloalkyl are unsubstituted or optionally independently substituted with one or more groups each independently selected from: halo, cyano, cycloalkyl, —O-cycloalkyl, alkyl, —O-alkyl;
   each R² (when present) is independently selected from the group consisting of —Si(CH₃)₃ and alkyl, wherein said alkyl is substituted with from 0 to 5 groups independently selected from: halo, —Si(R⁷)₃, —SF₅, —CHO, cycloalkyl, —O-cycloalkyl, —C(O)-cycloalkyl, —CO₂-cycloalkyl, —S(O)-cycloalkyl, —S(O)₂-cycloalkyl, —C(O)—N(R²⁰)-cycloalkyl, —S(O)—N(R²⁰)-cycloalkyl, —S(O)₂—N(R²⁰)-cycloalkyl, —C(O)-heterocycloalkyl, —S(O)₂-heterocycloalkyl, cycloalkenyl, —O-cycloalkenyl, —C(O)-cycloalkenyl, —CO₂-cycloalkenyl, —S(O)-cycloalkenyl, —S(O)₂-cycloalkenyl, —C(O)—N(R²⁰)-cycloalkenyl, —S(O)—N(R²⁰)-cycloalkenyl, —S(O)₂—N(R²⁰)-cycloalkenyl, —C(O)-heterocycloalkenyl, —S(O)₂-heterocycloalkenyl, alkyl, —O-alkyl, —C(O)-alkyl, —CO₂-alkyl, —S(O)-alkyl, —S(O)₂-alkyl, —C(O)—N(R²⁰)-alkyl, —S(O)—N(R²⁰)-alkyl, —S(O)₂—N(R²⁰)-alkyl, —C(O)-heteroalkyl, —S(O)₂-heteroalkyl, alkenyl, —O-alkenyl, —C(O)-alkenyl, —CO₂-alkenyl, —S(O)-alkenyl, —S(O)₂-alkenyl, —C(O)—N(R²⁰)-alkenyl, —S(O)—N(R²⁰)-alkenyl, —S(O)₂—N(R²⁰)-alkenyl, alkynyl, —O-alkynyl, —C(O)-alkynyl, —S(O)-alkynyl, —S(O)₂-alkynyl, —C(O)—N(R²⁰)-alkynyl, —S(O)—N(R²⁰)-alkynyl, —S(O)₂—N(R²⁰)-alkynyl, aryl, —O-aryl, —C(O)-aryl, —CO₂-aryl, —S(O)-aryl, —S(O)₂-aryl, —C(O)—N(R²⁰)-aryl, —S(O)—N(R²⁰)-aryl, —S(O)₂—N(R²⁰)-aryl,
wherein each of said alkyl, said alkenyl, said alkynyl, said cycloalkyl, said cycloalkenyl, said aryl, said heteroalkyl, said heterocycloalkyl, and said heterocycloalkenyl are unsubstituted or optionally independently substituted with one or more groups each independently selected from: —OH, halo, —Si(R⁷)₃, —CHO, cycloalkyl, cycloalkenyl, alkyl, alkenyl, alkynyl, aryl,
   R³ is selected from the group consisting of H and lower alkyl;

Z is a moiety selected from the group consisting of: —(CH₂)—(CH(CH₃))-C(O)OH, —(CH₂)—(CH₂)—(CH₂)—C(O)OH, —(CH₂)—C(CH₃)₂—C(O)OH, —(CH₂)—C(CH₃)(OH)—C(O)OH, —CH₂—CH₂—C(O)OH, —CH₂—CH(OH)—C(O)OH, —CH(CH₃)—CH₂—C(O)OH, —C(CH₃)₂—CH₂-C(O)OH, —CH₂—CH(F)-C(O)OH, —CH₂—CF₂-C(O)OH, —CH(CH₃)—CF₂—C(O)OH, —CH₂—CH₂—CF₂—C(O)OH, and

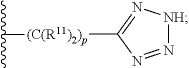

wherein p is an integer from 0 to 1, and R¹¹ (when present) is selected from the group consisting of H and lower alkyl;

each R^{5A} is independently selected from H, lower alkyl, -lower alkyl-Si(CH₃)₃, lower haloalkyl, and lower alkyl substituted with from 1 to 2 hydroxyl;

each R⁵ is independently selected from H, —OH, lower alkyl, -lower alkyl-Si(CH₃)₃, lower haloalkyl, and lower alkyl substituted with from 1 to 2 hydroxyl;

each R⁷ is independently selected from H, alkyl, heteroalkyl, and haloalkyl; and

each R²⁰ is independently selected from H, alkyl, haloalkyl, heteroalkyl, alkenyl, and alkynyl.

In one embodiment, the compounds of the invention have the general structure shown in Formula (II-a):

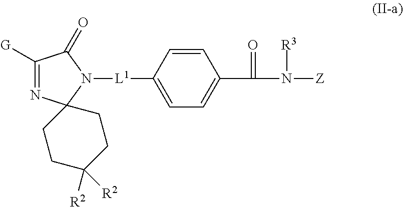

and include pharmaceutically acceptable salts, solvates, esters, prodrugs, tautomers, and isomers of said compounds,

wherein L¹, G, R³, Z, and each R² are selected independently of each other and as defined in Formula (II).

In one embodiment, the compounds of the invention have the general structure shown in Formula (II-b):

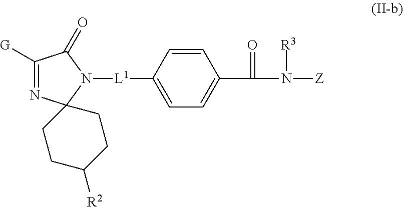

and include pharmaceutically acceptable salts, solvates, esters, prodrugs, tautomers, and isomers of said compounds,

wherein L¹, G, R², R³, and Z are selected independently of each other and as defined in Formula (II).

In one embodiment, in each of Formula (II), Formula (II-a), and Formula (II-b):

L¹ is selected from the group consisting of: a bond, straight or branched lower alkyl, and —CH(lower alkyl)- and —(CH(-lower alkyl-Si(CH₃)₃)—;
G is selected from the group consisting of: hydrogen, cycloalkyl, —N(R¹)cycloalkyl, heterocycloalkyl, alkyl, —N(R¹)-alkyl, heteroalkyl, —N(R¹)-heteroalkyl, alkenyl
   wherein said heterocycloalkyl and said heteroalkyl of G may be connected through any available carbon or heteroatom,
   and wherein said cycloalkyl, said alkenyl and said heterocycloalkyl of G are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
   wherein each of said alkyl, said cycloalkyl, and said aryl are unsubstituted or optionally independently substituted with one or more groups each independently selected from: halo, cyano, cycloalkyl, alkyl, —O-alkyl,
   and wherein said alkyl and said heteroalkyl of G is unsubstituted or substituted with one or more groups independently selected from: halo, cyano, cycloalkyl, —O-alkyl,
   wherein each of said alkyl and said cycloalkyl are unsubstituted or optionally independently substituted with one or more groups each independently selected from: halo, cyano, cycloalkyl, —O-cycloalkyl, alkyl, —O-alkyl,
      and wherein R¹ is independently selected from: hydrogen, cycloalkyl, heterocycloalkyl, alkyl, heteroalkyl,

wherein said heteroalkyl and said heterocycloalkyl of R¹ may be connected through any available carbon or heteroatom,

and wherein said cycloalkyl and said heterocycloalkyl of R¹ are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
wherein each of said alkyl, said cycloalkyl, and said aryl are unsubstituted or optionally independently substituted with one or more groups each independently selected from: halo, cyano, cycloalkyl, alkyl, —O-alkyl,
   and wherein said alkyl and said heteroalkyl of R¹ are unsubstituted or substituted with one or more groups independently selected from: halo, cyano, cycloalkyl, alkyl,
   wherein each of said alkyl and said cycloalkyl are unsubstituted or optionally independently substituted with one or more groups each independently selected from: halo, cyano, cycloalkyl, —O-cycloalkyl, alkyl, —O-alkyl,
      each R² is independently selected from the group consisting of H, straight or branched lower alkyl, and —Si(CH₃)₃;

R³ is selected from the group consisting of H and lower alkyl;

Z is a moiety selected from the group consisting of: —(CH₂)—(CH(CH₃))-C(O)OH, —(CH₂)—(CH₂)—(CH₂)—C(O)OH, —(CH₂)—C(CH₃)₂OC(O)OH, —(CH₂)—C(CH₃)(OH)—C(O)OH, —CH₂LH₂—C(O)OH, —CH₂—CH(OH)—C(O)OH, —CH(CH₃)—CH₂—C(O)OH, —C(CH₃)₂—CH₂-C(O)OH, —(C(R¹¹)₂)—(C(R¹⁴)₂)ₙ—C(O)OH, —CF₁₂—CH(F)—C(O)OH, —CH₂—CF₂—C(O)OH, —CH(CH₃)—CF₂—C(O)OH, —CH₂—CH₂—CF₂—C(O)OH, —(CH₂)—(CH(CH₃))—C(O)OCH₃, —(CH₂)—(CH₂)—(CH₂)—C(O)OCH₃, —(CH₂)—C(CH₃)₂—C(O)OCH₃, —(CH₂)—C(CH₃)(OH)—C(O)OCH₃, —CH₂.CH₂—C(O)OCH₃, —CH₂—CH(OH)—C(O)OCH₃, —CH(CH₃)—CH₂—C(O)OCH₃, —C(CH₃)₂—CH₂—C(O)OCH₃, —(C(R¹¹)₂)—(C(R¹⁴)₂), —C(O)OCH₃, —CH₂—CH(F)—C(O)OCH₃, —CH₂—CF₂—C(O)OCH₃, —CH(CH₃)—CF₂—C(O)OCH₃, —CH₂—CH₂—CF₂—C(O)OCH₃, and

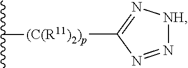

wherein p is an integer from 0 to 1, and R¹¹ (when present) is selected from the group consisting of H and lower alkyl;

each R⁵ is independently selected from H, —OH, lower alkyl, -lower alkyl-Si(CH₃)₃, lower haloalkyl, and lower alkyl substituted with from 1 to 2 hydroxyl; and

each R⁷ is independently selected from H, alkyl, heteroalkyl, and haloalkyl.

In one embodiment, in each of Formula (II), Formula (II-a), and Formula (II-b):

L¹ is selected from the group consisting of: a bond, straight or branched lower alkyl, —CH(lower alkyl)-, and —(CH(-lower alkyl-Si(CH₃)₃)—;

G is selected from morpholinyl,
wherein said morpholinyl of G is connected through nitrogen, and wherein said morpholinyl of G is unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
   wherein each of said alkyl, said cycloalkyl, and said aryl are unsubstituted or optionally independently substituted with one or more groups each independently selected from: halo, cyano, cycloalkyl, alkyl, —O-alkyl,
      each R² is independently selected from the group consisting of H, straight or branched lower alkyl, and —Si(CH₃)₃;

R³ is selected from the group consisting of H and lower alkyl;

Z is a moiety selected from the group consisting of: —(CH₂)—(CH(CH₃))—C(O)OH, —(CH₂)—(CH₂)—(CH₂)—C(O)OH, —(CH₂)—C(CH₃)₂—C(O)OH, —(CH₂)—C(CH₃)(OH)—C(O)OH, —CH₂—CH₂—C(O)OH, —CH₂—CH(OH)—C(O)OH, —CH(CH₃)—CH₂—C(O)OH, —C(CH₃)₂—CH₂—C(O)OH, —(C(R¹¹)₂)—(C(R¹⁴)₂)ₙ—C(O)OH, —CH₂—CH(F)—C(O)OH, —CH₂—CF₂—C(O)OH, —CH(CH₃)—CF₂—C(O)OH, —CH₂—CH₂—CF₂—C(O)OH, —(CH₂)—(CH(CH₃))—C(O)OCH₃, —(CH₂)—(CH₂)—(CH₂)—C(O)OCH₃, —(CH₂)—C(CH₃)₂—C(O)OCH₃, —(CH₂)—C(CH₃)(OH)—C(O)OCH₃, —CH₂.CH₂—C(O)OCH₃, —CH₂—CH(OH)—C(O)OCH₃, —CH(CH₃)—CH₂—C(O)OCH₃, —C(CH₃)₂—CH₂—C(O)OCH₃, —(C(R¹¹)₂)—(C(R¹⁴)₂)ₙ—C(O)OCH₃, —CH₂—CH(F)—C(O)OCH₃, —CH₂—CF₂—C(O)OCH₃, —CH(CH₃)—CF₂—C(O)OCH₃, —CH₂—CH₂—CF₂—C(O)OCH₃, and

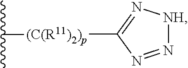

wherein p is an integer from 0 to 1, and R¹¹ (when present) is selected from the group consisting of H and lower alkyl;

each R⁵ is independently selected from H, —OH, lower alkyl, -lower alkyl-Si(CH₃)₃, lower haloalkyl, and lower alkyl substituted with from 1 to 2 hydroxyl; and

each R⁷ is independently selected from H, alkyl, heteroalkyl, and haloalkyl.

In one embodiment, in each of Formula (II), Formula (II-a), and Formula (II-b):

L¹ is selected from the group consisting of: a bond, straight or branched lower alkyl, and —CH(lower alkyl)-, and —(CH(-lower alkyl-Si(CH₃)₃)—;

G is selected from piperidinyl,
wherein said piperidinyl of G is connected through nitrogen, and wherein said piperidinyl of G is unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
   wherein each of said alkyl, said cycloalkyl, and said aryl are unsubstituted or optionally independently substituted with one or more groups each independently selected from: halo, cyano, cycloalkyl, alkyl, —O-alkyl,
      each R² is independently selected from the group consisting of H, straight or branched lower alkyl, and —Si(CH₃)₃;

R³ is selected from the group consisting of H and lower alkyl;

Z is a moiety selected from the group consisting of: —(CH₂)—(CH(CH₃))—C(O)OH, —(CH₂)—(CH₂)—(CH₂)—C(O)OH, —(CH₂)—C(CH₃)₂—C(O)OH, —(CH₂)—C(CH₃)(OH)—C(O)OH, —CH₂—CH₂—C(O)OH, —CH₂—CH(OH)—C(O)OH, —CH(CH₃)—CH₂—C(O)OH, —C(CH₃)₂—CH₂—C(O)OH, —(C(R¹¹)₂)—(C(R¹⁴)₂)ₙ—C(O)OH, —CH₂—CH(F)—C(O)OH, —CH₂—CF₂—C(O)OH, —CH(CH₃)—CF₂—C(O)OH, —CH₂—CH₂—CF₂—C(O)OH, —(CH₂)—(CH(CH₃))—C(O)OCH₃, —(CH₂)—(CH₂)—(CH₂)—C(O)OCH₃, —(CH₂)—C(CH₃)₂—C(O)OCH₃, —(CH₂)—C(CH₃)(OH)—C(O)OCH₃, —CH₂—CH₂—C(O)OCH₃, —CH₂—CH(OH)—C(O)OCH₃, —CH(CH₃)—CH₂—C(O)OCH₃, —C(CH₃)₂—CH₂—C(O)OCH₃, —(C(R¹¹)₂)—(C(R¹⁴)₂)ₙ—C(O)OCH₃, —CH₂—CH(F)—C(O)OCH₃, —CH₂—CF₂—C(O)OCH₃, —CH(CH₃)—CF₂—C(O)OCH₃, —CH₂—CH₂—CF₂—C(O)OCH₃, and

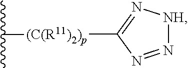

wherein p is an integer from 0 to 1, and R¹¹ (when present) is selected from the group consisting of H and lower alkyl;

each R⁵ is independently selected from H, —OH, lower alkyl, -lower alkyl-Si(CH₃)₃, lower haloalkyl, and lower alkyl substituted with from 1 to 2 hydroxyl; and

each R⁷ is independently selected from H, alkyl, heteroalkyl, and haloalkyl.

In one embodiment, in each of Formula (II), Formula (II-a), and Formula (II-b), L¹ is selected from the group consisting of: a bond,

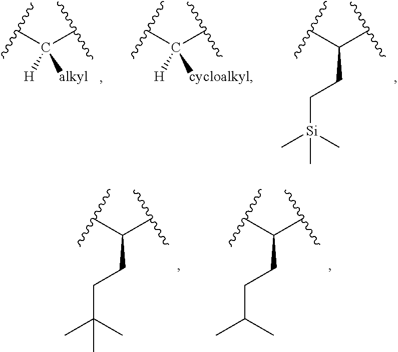

and —(CH₂)₁₋₃—. In one such embodiment, L¹ is selected from the group consisting of:

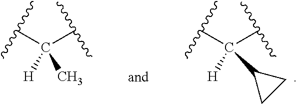

In one such embodiment, L¹ is

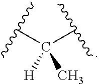

In one such embodiment, L¹ is

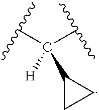

In one such embodiment, L¹ is

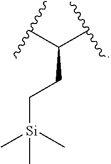

In one such embodiment, L¹ is

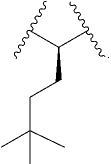

In one such embodiment, L¹ is

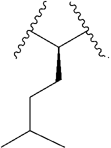

In one embodiment, in each of Formula (II), Formula (II-a), and Formula (II-b): L¹ is selected from the group consisting of:

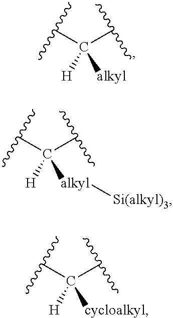

### and —(CH₂)₁₋₂—;

G is selected from the group consisting of: hydrogen, cycloalkyl, —N(R¹)cycloalkyl, heterocycloalkyl, alkyl, —N(R¹)-alkyl, heteroalkyl, —N(R¹)-heteroalkyl, alkenyl,
wherein said heterocycloalkyl and said heteroalkyl of G may be connected through any available carbon or heteroatom, and wherein said cycloalkyl, said alkenyl and said heterocycloalkyl of G are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
   wherein each of said alkyl, said cycloalkyl, and said aryl are unsubstituted or optionally independently substituted with one or more groups each independently selected from: halo, cyano, cycloalkyl, alkyl, —O-alkyl,
and wherein said alkyl and said heteroalkyl of G is unsubstituted or substituted with one or more groups independently selected from: halo, cyano, cycloalkyl, —O-alkyl,
   wherein each of said alkyl and said cycloalkyl are unsubstituted or optionally independently substituted with one or more groups each independently selected from: halo, cyano, cycloalkyl, —O-cycloalkyl, alkyl, —O-alkyl
      and wherein R¹ is independently selected from: hydrogen, cycloalkyl, heterocycloalkyl, alkyl, heteroalkyl,
wherein said heteroalkyl and said heterocycloalkyl of R¹ may be connected through any available carbon or heteroatom, and wherein said cycloalkyl and said heterocycloalkyl of R¹ are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
   wherein each of said alkyl, said cycloalkyl, and said aryl are unsubstituted or optionally independently substituted with one or more groups each independently selected from: halo, cyano, cycloalkyl, alkyl, —O-alkyl,
and wherein said alkyl and said heteroalkyl of R¹ are unsubstituted or substituted with one or more groups independently selected from: halo, cyano, cycloalkyl, alkyl,
   wherein each of said alkyl and said cycloalkyl are unsubstituted or optionally independently substituted with one or more groups each independently selected from: halo, cyano, cycloalkyl, —O-cycloalkyl, alkyl, —O-alkyl,
      each R² is independently selected from the group consisting of H, straight or branched lower alkyl, and —Si(CH₃)₃;

R³ is selected from the group consisting of H and lower alkyl; and

Z is selected from the group consisting of —CH₂—CH₂—C(O)OH and

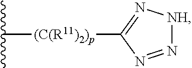

wherein p is and R¹¹ is H.
In one embodiment, in each of Formula (II), Formula (II-a), and Formula (II-b): L¹ is selected from the group consisting of:

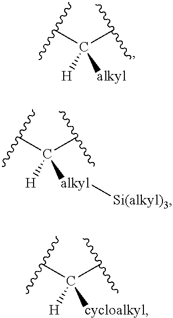

### and —(CH₂)₁₋₂—;

G is selected from morpholinyl,
wherein said morpholinyl of G is connected through nitrogen, and wherein said morpholinyl of G is unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
   wherein each of said alkyl, said cycloalkyl, and said aryl are unsubstituted or optionally independently substituted with one or more groups each independently selected from: halo, cyano, cycloalkyl, alkyl, —O-alkyl,
      each R² is independently selected from the group consisting of H, straight or branched lower alkyl, and —Si(CH₃)₃;

R³ is selected from the group consisting of H and lower alkyl; and

Z is selected from the group consisting of —CH₂—CH₂—C(O)OH and

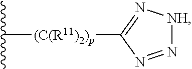

wherein p is and R¹¹ is H.

In one embodiment, in each of Formula (II), Formula (II-a), and Formula (II-b):

L¹ is selected from the group consisting of:

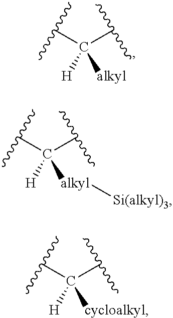

### and —(CH₂)₁₋₂—;

G is selected from piperidinyl,
wherein said piperidinyl of G is connected through nitrogen, and wherein said piperidinyl of G is unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
   wherein each of said alkyl, said cycloalkyl, and said aryl are unsubstituted or optionally independently substituted with one or more groups each independently selected from: halo, cyano, cycloalkyl, alkyl, —O-alkyl,
      each R² is independently selected from the group consisting of H, straight or branched lower alkyl, and —Si(CH₃)₃;

R³ is selected from the group consisting of H and lower alkyl; and

Z is selected from the group consisting of —CH₂—CH₂—C(O)OH and

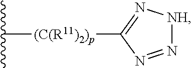

wherein p is 1 and R¹¹ is H.

In one embodiment, in each of Formula (II), Formula (II-a), and Formula (II-b):

L¹ is selected from the group consisting of

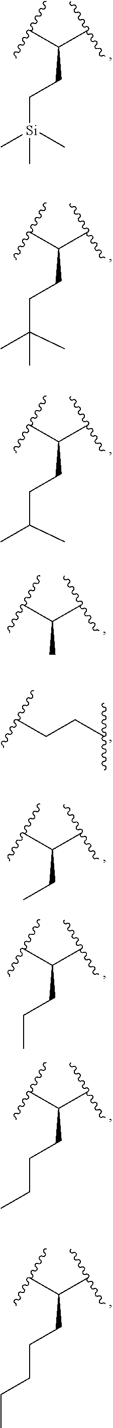

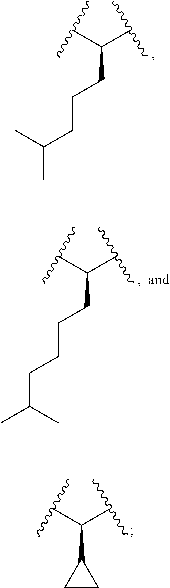

and
G is selected from the group consisting of: hydrogen, cycloalkyl, —N(R¹)cycloalkyl, heterocycloalkyl, alkyl, —N(R¹)-alkyl, heteroalkyl, —N(R¹)-heteroalkyl, alkenyl
   wherein said heterocycloalkyl and said heteroalkyl of G may be connected through any available carbon or heteroatom,
   and wherein said cycloalkyl, said alkenyl and said heterocycloalkyl of G are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
   wherein each of said alkyl, said cycloalkyl, and said aryl are unsubstituted or optionally independently substituted with one or more groups each independently selected from: halo, cyano, cycloalkyl, alkyl, —O-alkyl,
   and wherein said alkyl and said heteroalkyl of G is unsubstituted or substituted with one or more groups independently selected from: halo, cyano, cycloalkyl, —O-alkyl,
   wherein each of said alkyl and said cycloalkyl are unsubstituted or optionally independently substituted with one or more groups each independently selected from: halo, cyano, cycloalkyl, —O-cycloalkyl, alkyl, —O-alkyl,
      and wherein R¹ is independently selected from: hydrogen, cycloalkyl, heterocycloalkyl, alkyl, heteroalkyl,

wherein said heteroalkyl and said heterocycloalkyl of R¹ may be connected through any available carbon or heteroatom,

and wherein said cycloalkyl and said heterocycloalkyl of R¹ are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
wherein each of said alkyl, said cycloalkyl, and said aryl are unsubstituted or optionally independently substituted with one or more groups each independently selected from: halo, cyano, cycloalkyl, alkyl, —O-alkyl,
and wherein said alkyl and said heteroalkyl of R¹ are unsubstituted or substituted with one or more groups independently selected from: halo, cyano, cycloalkyl, alkyl,
wherein each of said alkyl and said cycloalkyl are unsubstituted or optionally independently substituted with one or more groups each independently selected from: halo, cyano, cycloalkyl, —O-cycloalkyl, alkyl, —O-alkyl,

each R² is independently selected from the group consisting of iso-propyl, fed-butyl and tert-pentyl;

R³ is H; and

Z is selected from the group consisting of —CH₂—CH₂—C(O)OH and

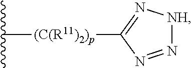

wherein p is 1 and R¹¹ is H.

In one embodiment, in each of Formula (II), Formula (II-a), and Formula (II-b):

L¹ is selected from the group consisting of

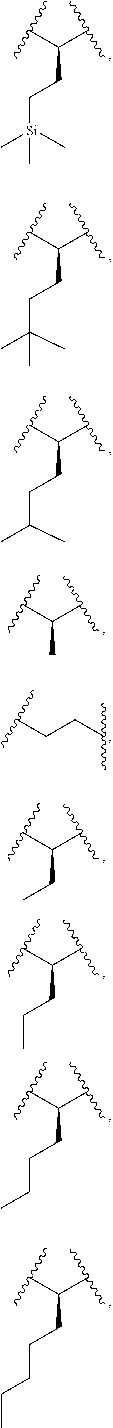

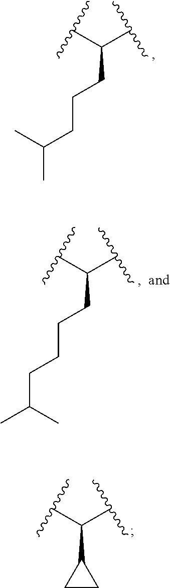

and

G is selected from morpholinyl,
wherein said morpholinyl of G is connected through nitrogen, and wherein said morpholinyl of G is unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
   wherein each of said alkyl, said cycloalkyl, and said aryl are unsubstituted or optionally independently substituted with one or more groups each independently selected from: halo, cyano, cycloalkyl, alkyl, —O-alkyl,

each R² is independently selected from the group consisting of iso-propyl, tert-butyl and tert-pentyl;

R³ is H; and

Z is selected from the group consisting of —CH₂—CH₂—C(O)OH and

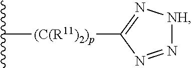

wherein p is 1 and R¹¹ is H.

In one embodiment, in each of Formula (II), Formula (II-a), and Formula (II-b):

L¹ is selected from the group consisting of

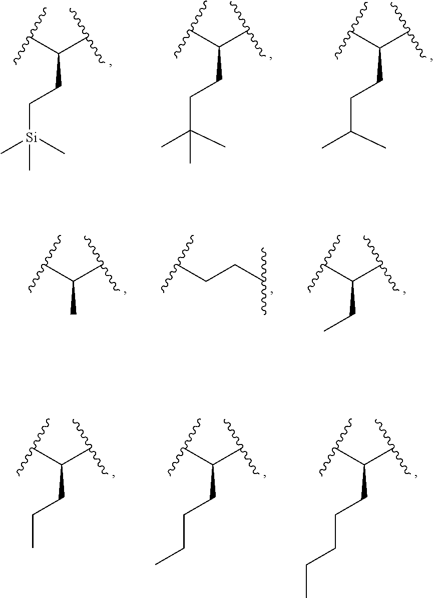

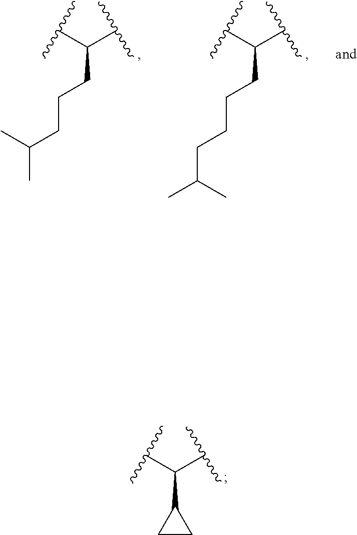

and

G is selected from piperidinyl,
wherein said piperidinyl of G is connected through nitrogen, and wherein said piperidinyl of G is unsubstituted or substituted with one or more groups independently selected from: halo, cyano, cycloalkyl, —O-cycloalkyl, —C(O)-cycloalkyl, alkyl, —O-alkyl, —C(O)-alkyl, and aryl,
   wherein each of said alkyl, said cycloalkyl, and said aryl are unsubstituted or optionally independently substituted with one or more groups each independently selected from: halo, cyano, cycloalkyl, alkyl, —O-alkyl,

each R² is independently selected from the group consisting of iso-propyl, tert-butyl, and tert-pentyl;

R³ is H; and

Z is selected from the group consisting of —CH₂—CH₂—C(O)OH and

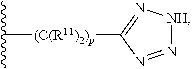

wherein p is 1 and R¹¹ is H.

In one embodiment, the compounds of the invention have the general structure shown in the tables below, and include pharmaceutically acceptable salts, solvates, esters, prodrugs, tautomers, and isomers of said compounds.

In the various embodiments described herein, variables of each of the general formulas not explicitly defined in the context of the respective formula are as defined in Formula (A).

In one embodiment, a compound or compounds of the invention is/are in isolated or purified form.

The terms used herein have their ordinary meaning and the meaning of such terms is independent at each occurrence thereof. That notwithstanding and except where stated otherwise, the following definitions apply throughout the specification and claims. Chemical names, common names and chemical structures may be used interchangeably to describe that same structure. These definitions apply regardless of whether a term is used by itself or in combination with other terms, unless otherwise indicated. Hence the definition of “alkyl” applies to “alkyl” as well as the “alkyl” portion of “hydroxyalkyl”, “haloalkyl”, arylalkyl-, alkylaryl-, “alkoxy” etc.

“Mammal” means humans and other mammalian animals.

A “patient” is a human or non-human mammal. In one embodiment, a patient is a human. In another embodiment, a patient is a non-human mammal, including, but not limited to, a monkey, baboon, mouse, rat, horse, dog, cat or rabbit. In another embodiment, a patient is a companion animal, including but not limited to a dog, cat, rabbit, horse or ferret. In one embodiment, a patient is a dog. In another embodiment, a patient is a cat.

The term “obesity” as used herein, refers to a patient being overweight and having a body mass index (BMI) of 25 or greater. In one embodiment, an obese patient has a BMI of 25 or greater. In another embodiment, an obese patient has a BMI from 25 to 30. In another embodiment, an obese patient has a BMI greater than 30. In still another embodiment, an obese patient has a BMI greater than 40.

The term “impaired glucose tolerance” (IGT) as used herein, is defined as a two-hour glucose level of 140 to 199 mg per dL (7.8 to 11.0 mmol) as measured using the 75-g oral glucose tolerance test. A patient is said to be under the condition of impaired glucose tolerance when he/she has an intermediately raised glucose level after 2 hours, wherein the level is less than would qualify for type 2 diabetes mellitus.

The term “impaired fasting glucose” (IFG) as used herein, is defined as a fasting plasma glucose level of 100 to 125 mg/dL; normal fasting glucose values are below 100 mg per dL.

The term “effective amount” as used herein, refers to an amount of Compound of Formula (I) and/or an additional therapeutic agent, or a composition thereof that is effective in producing the desired therapeutic, ameliorative, inhibitory or preventative effect when administered to a patient suffering from a Condition. In the combination therapies of the present invention, an effective amount can refer to each individual agent or to the combination as a whole, wherein the amounts of all agents administered are together effective, but wherein the component agent of the combination may not be present individually in an effective amount.

“Halogen” means fluorine, chlorine, bromine, or iodine, Preferred are fluorine, chlorine and bromine.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being as described herein. Nonlimiting examples of suitable alkyl groups include methyl, ethyl, n-propyl, isopropyl and t-butyl. Additional nonlimiting examples of branched lower alkyl include -loweralkyl-isopropyl, (e.g., —CH₂CH₂CH(CH₃)₂), -lower alkyl-t-butyl (e.g., —CH₂CH₂C(CH₃)₃).

The term “haloalkyl” as used herein, refers to an alkyl group, as defined above, wherein one or more of the alkyl group's hydrogen atoms have been independently replaced with —F, —Cl, —Br or —I. Non-limiting illustrative examples of haloalkyl groups include —CH₂F, —CHF₂, —CF₃, —CH₂CHF₂, —CH₂CF₃, —CCl₃, —CHCl₂, —CH₂Cl, and —CH₂CHCl₃.

The term “deuterioalkyl” (or “deuteroalkyl”) as used herein, refers to an alkyl group, as defined above, wherein one or more of the alkyl group's hydrogen atoms have been independently replaced with deuterium.

“Heteroalkyl” means an alkyl moiety as defined above, having one or more carbon atoms, for example one, two or three carbon atoms, replaced with one or more heteroatoms, which may be the same or different, where the point of attachment to the remainder of the molecule is through a carbon atom of the heteroalkyl radical. Suitable such heteroatoms include O, S, S(O), S(O)₂, and —NH—, —N(alkyl)-. Non-limiting examples include ethers, thioethers, amines, 2-aminoethyl, 2-dimethylaminoethyl, and the like.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched.

“Alkenyl” may be unsubstituted or optionally substituted by one or more substituents which may be the same or different, each substituent being as described herein. Non-limiting examples of suitable alkenyl groups include ethenyl, propenyl, n-butenyl, 3-methylbut-2-enyl, n-pentenyl, octenyl and decenyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being as described herein.

“Aryl” means an aromatic monocyclic or multicyclic ring system comprising about 6 to about 14 carbon atoms, preferably about 6 to about 10 carbon atoms. The aryl group may be unsubstituted or optionally substituted by one or more substituents which may be the same or different, each substituent being as described herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” may be unsubstituted or optionally substituted by one or more substituents which may be the same or different, each substituent being as described herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide, “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like. As noted elsewhere, the “heteroaryl” group may be bound to the parent moiety through an available carbon or nitrogen atom.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl may be unsubstituted or optionally substituted by one or more substituents which may be the same or different, each substituent being as described herein. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, 2-decalinyl, norbornyl, adamantyl and the like.

Further non-limiting examples of suitable multicyclic cycloalkyl groups include the moieties:

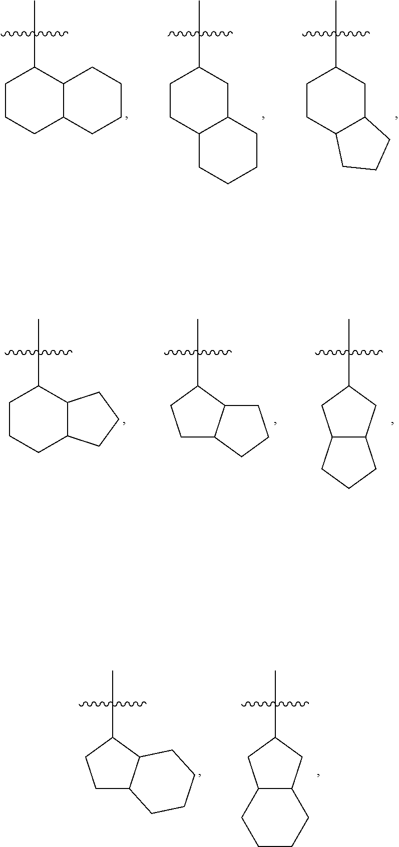

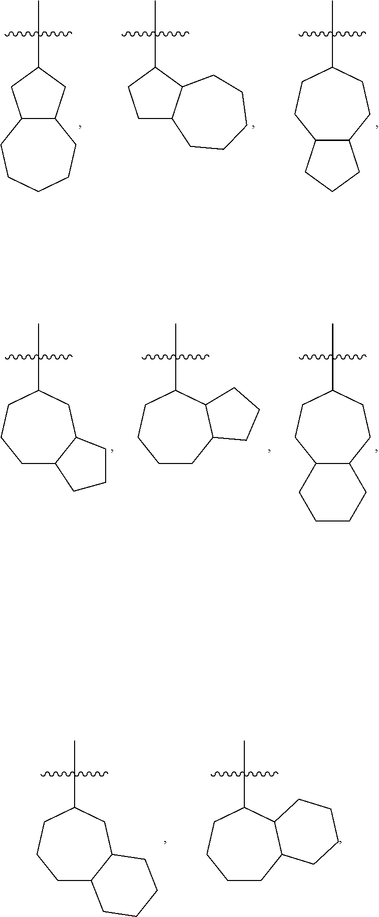

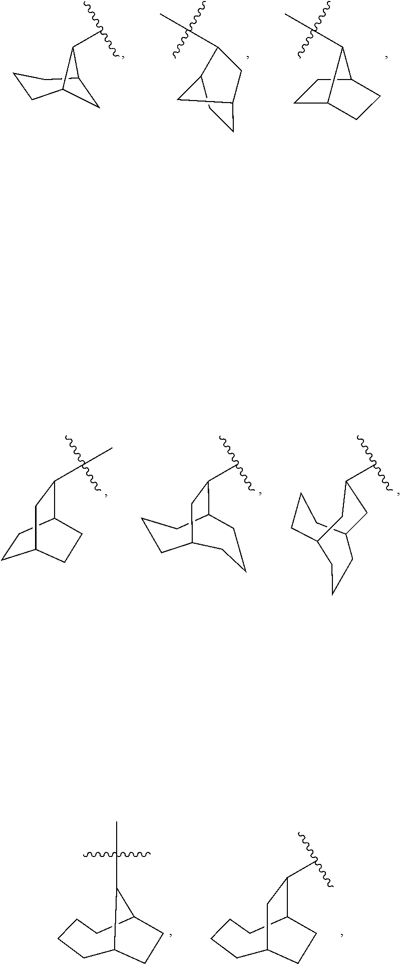

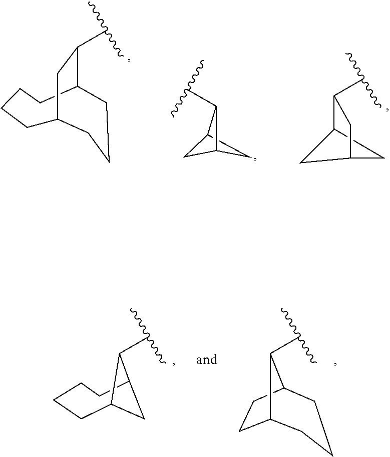 [SMILES: *C1CC2CCCCC1CCC2 ; *C1C2CC1C2 ; *C1CC2CC1C2 ; *C1C2CCCCC1C2 ; *C1C2CCCC1CC2]

and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl may be unsubstituted or optionally substituted by one or more substituents which may be the same or different, each substituent being as described herein. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Heterocycloalkyl” (or “heterocyclyl”) means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl may be unsubstituted or optionally substituted by one or more substituents which may be the same or different, each substituent being as described herein. The nitrogen or sulfur atom of the heterocyclyl can be optionally oxidized to the corresponding N-oxide, S-oxide or S,S-dioxide. Thus, the term “oxide,” when it appears in a definition of a variable in a general structure described herein, refers to the corresponding N-oxide, S-oxide, or S,S-dioxide. Non-limiting examples of suitable monocyclic heterocyclyl rings include diazapanyl, piperidinyl, pyrrolidinyl, piperazinyl, morpholinyl, thiomorpholinyl, thiazolidinyl, 1,4-dioxanyl, tetrahydrofuranyl, tetrahydropyranyl, tetrahydrothiophenyl, lactam, lactone, and the like. Non-limiting examples of suitable multicyclic heterocycloalkyl include

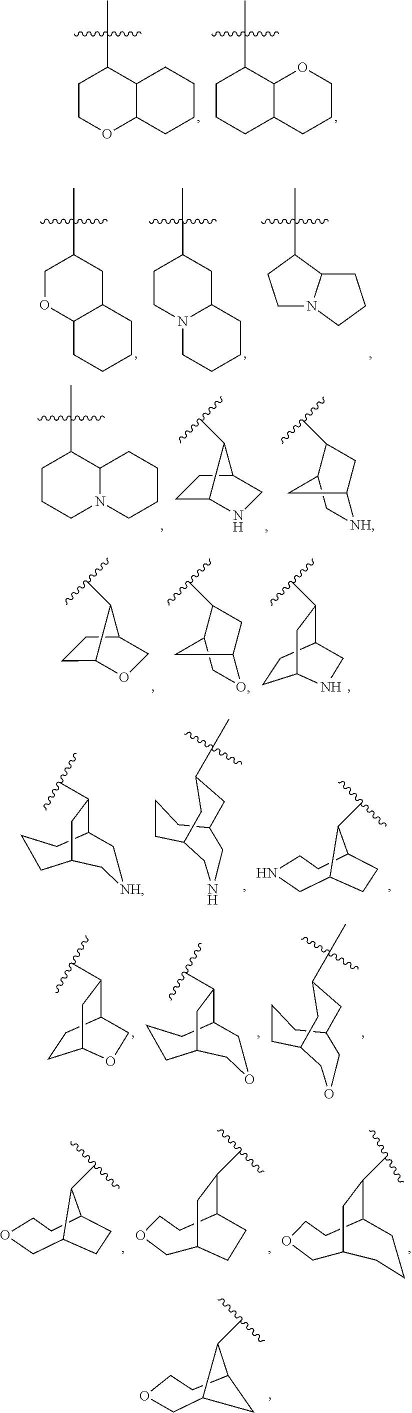

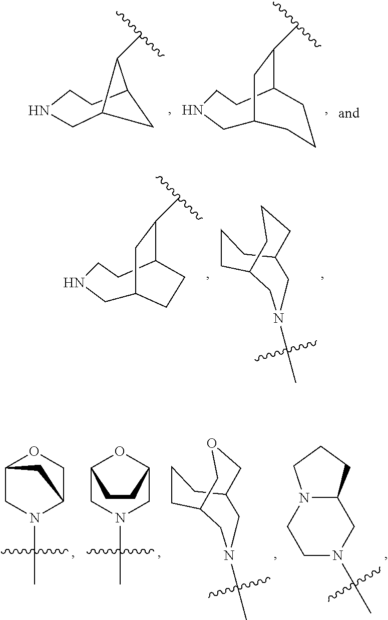

and the like. “Heterocycloalkyl” also includes rings wherein ═O replaces two available hydrogens on the same carbon atom (i.e., heterocyclyl includes rings having a carbonyl group in the ring). Such ═O groups may be referred to herein as “oxo.” Example of such moiety is pyrrolidinone (or pyrrolidone):

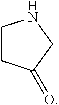

“Heterocycloalkenyl” (or “heterocyclenyl”) means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocycloalkenyl may be unsubstituted or optionally substituted by one or more substituents which may be the same or different, each substituent being as described herein. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” also includes rings wherein ═O replaces two available hydrogens on the same carbon atom (i.e., heterocyclyl includes rings having a carbonyl group in the ring). Example of such moiety is pyrrolidenone (or pyrrolone):

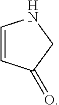

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

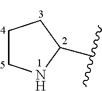 [SMILES: *C1CCCN1]

there is no —OH attached directly to carbons marked 2 and 5.

It should also be noted that tautomeric forms such as, for example, the moieties:

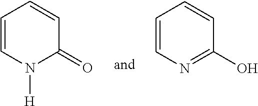 [SMILES: O=c1cccc[nH]1 ; Oc1ccccn1]

are considered equivalent in certain embodiments of this invention. Thus, for example, when a compound of the invention contains a

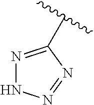 [SMILES: *c1nn[nH]n1]

group,

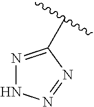 [SMILES: *c1nn[nH]n1]

is equivalent to

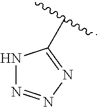 [SMILES: *c1nnn[nH]1]

It should be understood that for hetero-containing functional groups described herein, e.g., heterocycloalkyl, heterocycloalkenyl, heteroalkyl, and heteroaryl the bond to the parent moiety can be through an available carbon or heteroatom (e.g., nitrogen atom).

“Aralkyl” or “arylalkyl” means an aryl-alkyl-group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl. The term (and similar terms) may be written as “arylalkyl-” to indicate the point of attachment to the parent moiety.

Similarly, “heteroarylalkyl”, “cycloalkylalkyl”, “cycloalkenylalkyl”, “heterocycloalkylalkyl”, “heterocycloalkenylalkyl”, etc., mean a heteroaryl, cycloalkyl, cycloalkenyl, heterocycloalkyl, heterocycloalkenyl, etc. as described herein bound to a parent moiety through an alkyl group. Preferred groups contain a lower alkyl group. Such alkyl groups may be straight or branched, unsubstituted and/or substituted as described herein.

Similarly, “arylfused arylalkyl-”, arylfused cycloalkylalkyl-, etc., means an arylfused aryl group, arylfused cycloalkyl group, etc. linked to a parent moiety through an alkyl group. Preferred groups contain a lower alkyl group. Such alkyl groups may be straight or branched, unsubstituted and/or substituted as described herein.

“Alkylaryl” means an alkyl-aryl-group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl, adamantylpropyl, and the like.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclylalkyl” (or “heterocycloalkylalkyl”) means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenylalkyl” means a heterocyclenyl moiety as defined above linked via an alkyl moiety (defined above) to a parent core.

“Heteroaralkyl” means a heteroaryl-alkyl-group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl-group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)-group in which the aryl group is as previously described. The bond to the parent moiety is through the carbonyl. Non-limiting examples of suitable groups include benzoyl and 1-naphthoyl.

“Heteroaroyl” means an heteroaryl-C(O)-group in which the heteroaryl group is as previously described. The bond to the parent moiety is through the carbonyl. Non-limiting examples of suitable groups include pyridoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Alkyoxyalkyl” means a group derived from an alkoxy and alkyl as defined herein. The bond to the parent moiety is through the alkyl.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” (or “arylalkyloxy”) means an aralkyl-O— group (an arylaklyl-O-group) in which the aralkyl group is as previously described. Non-limiting examples of suitable aralkyloxy groups include benzyloxy and 1- or 2-naphthalenemethoxy. The bond to the parent moiety is through the ether oxygen.

“Arylalkenyl” means a group derived from an aryl and alkenyl as defined herein. Preferred arylalkenyls are those wherein aryl is phenyl and the alkenyl consists of about 3 to about 6 atoms. The bond to the parent moiety is through a non-aromatic carbon atom.

“Arylalkynyl” means a group derived from a aryl and alkenyl as defined herein. Preferred arylalkynyls are those wherein aryl is phenyl and the alkynyl consists of about 3 to about 6 atoms. The bond to the parent moiety is through a non-aromatic carbon atom.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—C(O)-group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)-group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)-group. Non-limiting example of a suitable aralkoxycarbonyl group is benzyloxycarbonyl. The bond to the parent moiety is through the carbonyl.

“Alkylsulfonyl” means an alkyl-S(O₂)-group. Preferred groups are those in which the alkyl group is lower alkyl. The bond to the parent moiety is through the sulfonyl.

“Arylsulfonyl” means an aryl-S(O₂)-group. The bond to the parent moiety is through the sulfonyl.

“Spirocycloalkyl” means a monocyclic or multicyclic cycloalkyl group attached to a parent moiety by replacement of two available hydrogen atoms attached to the same carbon atom. The spirocycloalkyl may optionally be substituted as described herein. Non-limiting examples of suitable monocyclic spirocycloalkyl groups include spirocyclopropyl, spirolcyclobutyl, spirocyclopentyl, spirocyclohexyl, spirocycloheptyl, and spirocyclooctyl. Non-limiting examples of suitable multicyclic spirocycloalkyl groups include the moieties

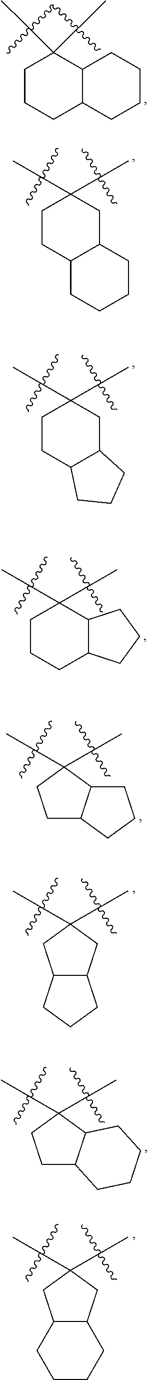

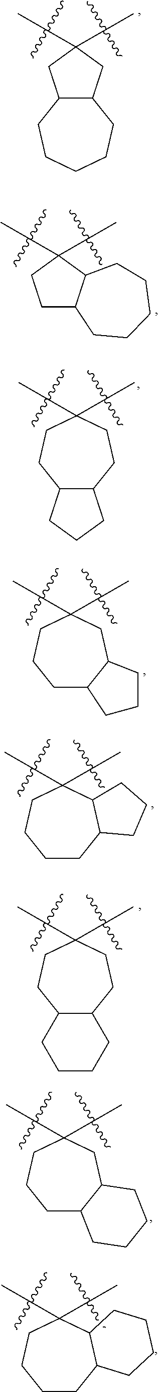

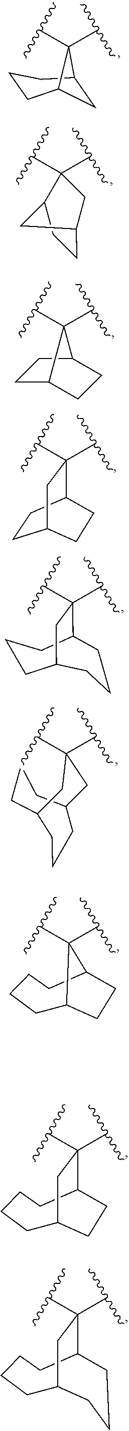

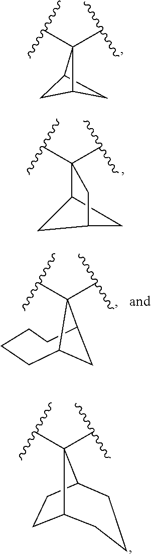 [SMILES: *C1(*)C2CC1C2 ; *C1(*)CC2CC1C2 ; *C1(*)C2CCCCC1C2 ; *C1(*)C2CCCC1CC2]

and the like.

“Spirocycloalkenyl” means a spirocycloalkyl group which contains at least one carbon-carbon double bond. Preferred spirocycloalkenyl rings contain about 5 to about 7 ring atoms. The spirocycloalkenyl can be optionally substituted as described herein. Non-limiting examples of suitable monocyclic cycloalkenyls include spirocyclopentenyl, spirocyclohexenyl, spirocyclohepta-1,3-dienyl, and the like. Non-limiting example of a suitable multicyclic spirocycloalkenyl include

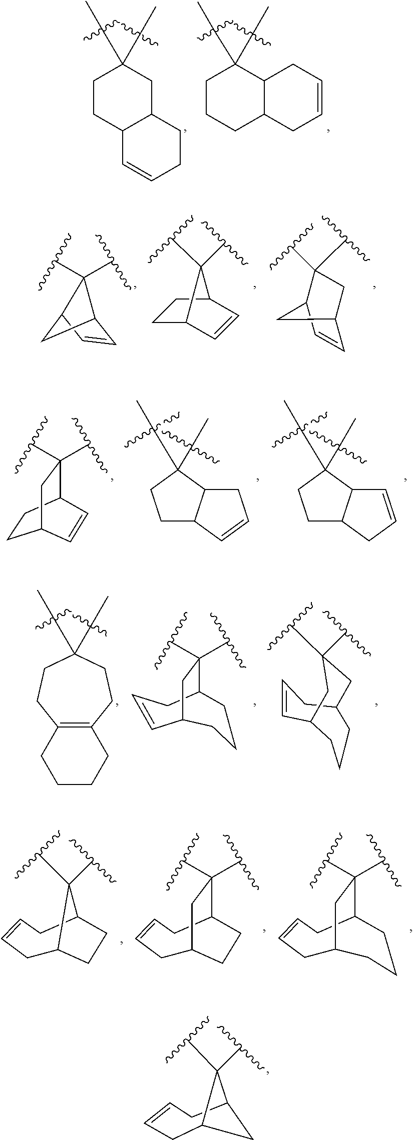

and the like.

“Sprioheterocycloalkyl” means a monocyclic or multicyclic heterocycloalkyl group (include oxides thereof) attached to the parent moiety by replacement of two available hydrogen atoms attached to the same carbon atom. The spiroheterocycloalkyl may be optionally substituted as described herein. Non-limiting examples of suitable multicyclic spiroheterocycloalkyl include

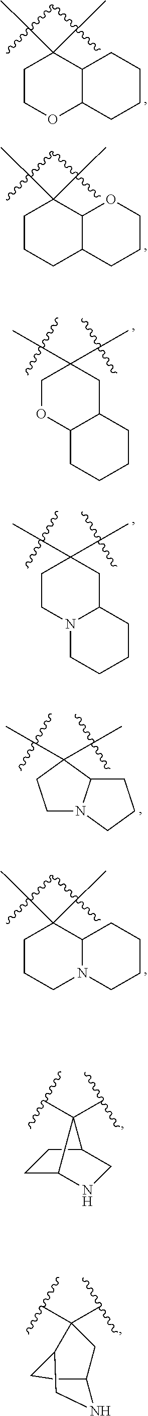

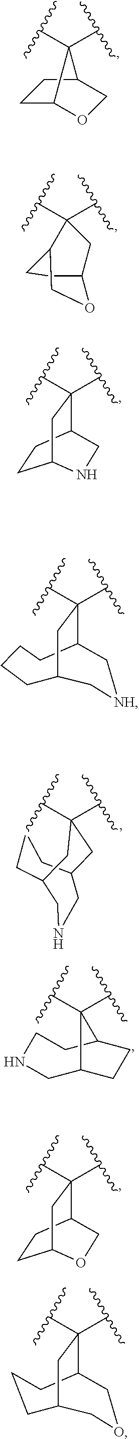

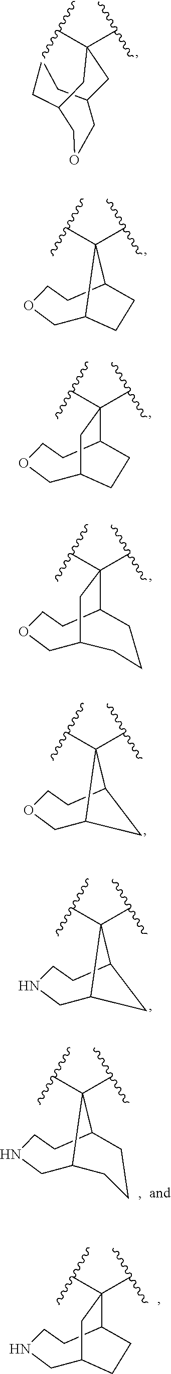

and the like.

“Spiroheterocycloalkenyl” (or “spiroheterocyclenyl”) means a spiroheterocycloalkyl group which contains at least one carbon-carbon double bond. Non-limiting examples of suitable multicyclic spiroheterocycloalkenyl include:

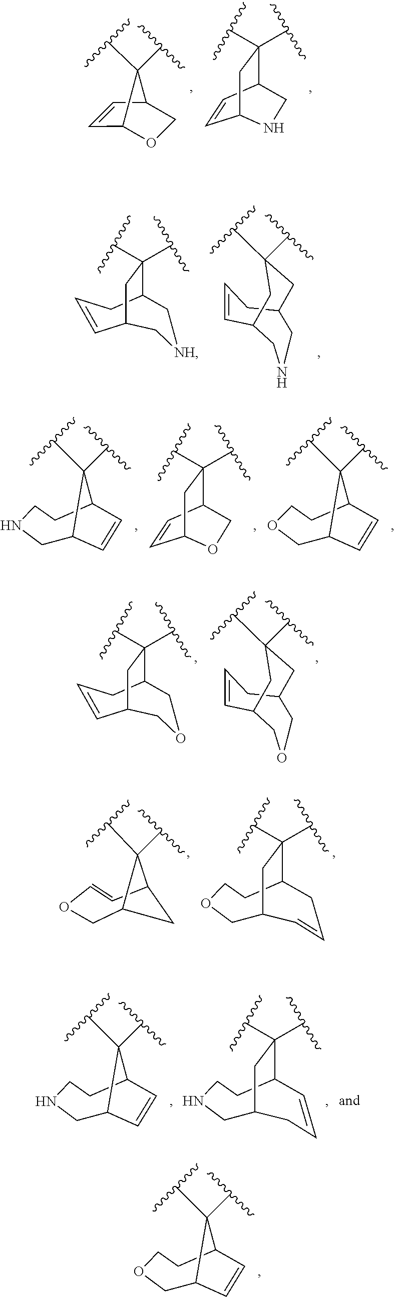

and the like.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. The terms “stable compound” or “stable structure” mean a compound that is sufficiently robust to survive isolation to a useful degree of purity from a reaction mixture, and formulation into an efficacious therapeutic agent.

The term “optionally substituted” means optional substitution with the specified groups, radicals or moieties.

Substitution on a cycloalkylalkyl, heterocycloalkylalkyl, arylalkyl, heteroarylalkyl, arylfused cycloalkylalkyl-moiety or the like includes substitution on any ring portion and/or on the alkyl portion of the group.

The term, “compound(s) of the invention,” as used herein, refers, collectively or independently, to any of the compounds embraced by the general formulas described herein, e.g., Formula (A), Formula (A-1), Formula (A-1a), Formula (A-1b), Formula (A-2a), Formula (A-2b), Formula (A-2c), Formula (A-2d), Formula (I), Formula (I-1), Formula (II), Formula (II-a), and Formula (II-b), and the example compounds thereof. When a variable appears more than once in a group, e.g., alkyl in —N(alkyl)₂, or a variable appears more than once in a structure presented herein these formulas, the variables can be the same or different.

With reference to the number of moieties (e.g., substituents, groups or rings) in a compound, unless otherwise defined, the phrases “one or more” and “at least one” mean that there can be as many moieties as chemically permitted, and the determination of the maximum number of such moieties is well within the knowledge of those skilled in the art. With respect to the compositions and methods comprising the use of “at least one compound of the invention, e.g., of Formula (I),” one to three compounds of the invention, e.g., of Formula (I) can be administered at the same time, preferably one.

As used herein, the term “composition” is intended to encompass a product comprising the specified ingredients in the specified amounts, as well as any product which results, directly or indirectly, from combination of the specified ingredients in the specified amounts.

The line ----, as a bond generally indicates a mixture of, or either of, the possible isomers, e.g., containing (R)- and (S)-stereochemistry. For example:

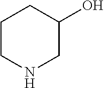

means containing both

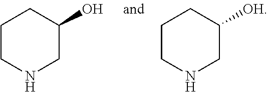 [SMILES: O[C@@H]1CCCNC1 ; O[C@H]1CCCNC1]

In the structure

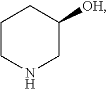

the

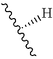

is implied. Thus, the structure

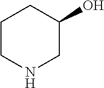

is equivalent to

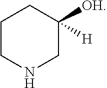

Similarly, and by way of additional non-limiting example, when -L₁- is

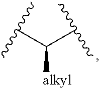

the

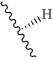

is implied. Thus,

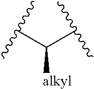

is equivalent to

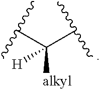

The wavy line [symbol] , as used herein, indicates a point of attachment to the rest of the compound. For example, each wavy line in the following structure:

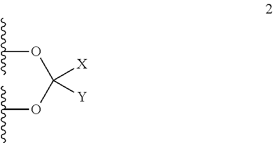

indicates a point of attachment to the core structure, as described herein. Lines drawn into the ring systems, such as, for example:

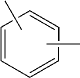

indicate that the indicated line (bond) may be attached to any of the substitutable ring carbon atoms.

“Oxo” is defined as a oxygen atom that is double bonded to a ring carbon in a cycloalkyl, cycloalkenyl, heterocyclyl, heterocyclenyl, or other ring described herein, e.g.,

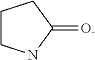

In the compounds of the invention, where there are multiple oxygen and/or sulfur atoms in a ring system, there cannot be any adjacent oxygen and/or sulfur present in said ring system.

It is noted that the carbon atoms for compounds of the invention may be replaced with 1 to 3 silicon atoms so long as all valency requirements are satisfied.

As well known in the art, a bond drawn from a particular atom wherein no moiety is depicted at the terminal end of the bond indicates a methyl group bound through that bond to the atom, unless stated otherwise. For example:

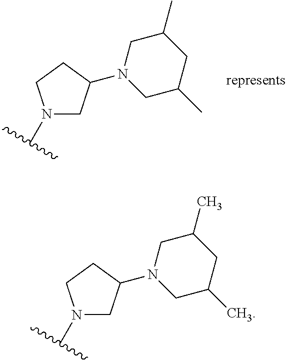 [SMILES: *N1CCC(N2CC(C)CC(C)C2)C1 ; *N1CCC(N2CC(C)CC(C)C2)C1]

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1999), Wiley, New York.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of the invention or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of the invention or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a compound of the invention contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of the invention incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or an unnatural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N- or di-N,N—(C₁-C₆)alkylaminoalkyl, —C(Y⁴)Y⁵ wherein Y⁴ is H or methyl and Y⁵ is mono-N- or di-N,N—(C₁-C₆)alkylamino morpholino, piperidin-1-yl or pyrrolidin-1-yl, and the like.

Compounds of the invention wherein Z is an ester moiety, such as those selected from —(C(R¹¹)₂)—(C(R¹²R¹³))ₘ—C(O)Oalkyl, and —(C(R¹¹)₂)—(C(R¹⁴)₂)ₙ—C(O)Oalkyl, are also expected to form prodrugs. Such prodrugs are included in the compounds of the invention.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al., AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of the invention can form salts which are also within the scope of this invention. Reference to a compound of the invention herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of the invention contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the invention may be formed, for example, by reacting a compound of the invention with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson at al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quaternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of the invention, and salts, solvates, esters and prodrugs thereof, may exist in their tautomeric form (for example, as an amide or imino ether). All such tautomeric forms are contemplated herein as part of the present invention.

The compounds of the invention may contain asymmetric or chiral centers, and, therefore, exist in different stereoisomeric forms. It is intended that all stereoisomeric forms of the compounds of the invention as well as mixtures thereof, including racemic mixtures, form part of the present invention. In addition, the present invention embraces all geometric and positional isomers. For example, if a compound of the invention incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of the invention may be atropisomers (e.g., substituted biaryls) and are considered as part of this invention. Enantiomers can also be separated by use of chiral HPLC column.

It is also possible that the compounds of the invention may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of the invention incorporates a double bond or a fused ring, both the cis- and trans-forms, as well as mixtures, are embraced within the scope of the invention. Also, for example, all keto-enol and imine-enamine forms of the compounds are included in the invention).

By way of further non-limiting example, compounds of the invention having the general structure shown in Formula (II-b):

In one embodiment, the compounds of the invention have the general structure shown in Formula (II-b):

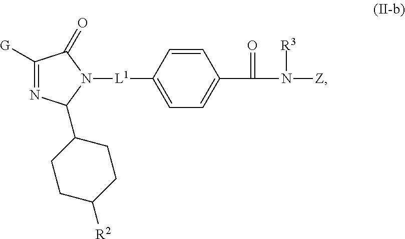

and encompass compounds of the formula:

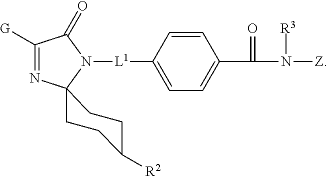

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, 14C, 15N, ¹⁸O, ¹⁷O, ³¹P, ³²P, ³⁵S, ¹⁸F, and ³⁸Cl, respectively.

Certain isotopically-labelled compounds of the invention (e.g., those labeled with ³H and ¹⁴C) are useful in compound and/or substrate tissue distribution assays. Tritiated (Le., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of the invention can generally be prepared by following procedures analogous to those disclosed in the Schemes and/or in the Examples hereinbelow, by substituting an appropriate isotopically labelled reagent for a non-isotopically labelled reagent. Such compounds are within the scope of the compounds of the invention.

Polymorphic forms of the compounds of the invention, and of the salts, solvates, esters and prodrugs of the compounds of the invention, are intended to be included in the present invention.

### EXPERIMENTAL SECTION

Abbreviations used in the experimental section may include but are not limited to the following:
AGN Acetonitrile
AcOH Acetic acid
Aq Aqueous
Bn Benzyl
BOC Cert-Buto)rycarbonyl
BOC₂O BOC Anhydride
Bu Butyl
C (or ° C.) degrees Celsius
Cbz benzyloxycarbonyl
DBU 1,8-Diazabicyclo[5.4.0]undec-7-ene
DCM Dichloromethane
DIPEA Diisopropylethylamine
DMA N,N-Dimethylacetamide
DMAP 4-Dimethylaminopyridine
DME 1,2-dimethoxyethane
DMF Dimethylformamide
DMSO Dimethyl sulfoxide
DPPF 1,1′-(bis-diphenylphosphino) ferrocene
EDCI 1-(3-Dimethylaminopropyl)-3-ethylcarbodiimide hydrochloride
EDC 1-(3-Dimethylaminopropyl)-3-ethylcarbodiimide hydrochloride
El Electron ionization
Eq Equivalents
Et Ethyl
EtOAc Ethyl acetate
EtOH Ethanol
g grams
h hours
hr hours
¹H proton
HATU N,N,N′,N′-Tetramethyl-O-(7-Azabenzotriazol-1-yl)Uroniurn hexafluorophosphate
Hex hexanes
HOBT 1-Hydroxybenzotriazole
HOBT.H₂O 1-Hydroxybenzotriazole hydrate
HOTs para-toluene sulfonic acid (see also TsOH)
HOTs.H₂O para-toluene sulfonic acid hydrate (see also TsOH.H₂O)
HMPA hexamethylphosphoramide
HPLC High pressure liquid chromatography
IPA isopropanol, 2-propanol
LDA lithium diisopropylamide
M Molar
mmol milimolar
mCPBA meta-Chloroperoxybenzoic acid
Me Methyl
MeCN Acetonitrile
MeOH Methanol
min Minutes
mg Milligrams
MHZ Megahertz
mL (or ml) Milliliter
mol sieves molecular sieves
N normal
NMR Nuclear Magnetic Resonance
MS Mass Spectroscopy
NBS N-Bromosuccinimide
NMM N-Methylmorpholine
NMP 1-methyl-2-pyrrolidone
ON Overnight
PTLC Preparative thin layer chromatography
PyBrOP Bromo-tris-pyrrolidino-phosphonium hexafluorophosphate
PyBOP (Benzotriazol-1-yloxy)tripyrrolidinophosphonium hexa-fluorophosphate
Pyr Pyridine
Quant quantitative
RT or rt Room temperature sat (or sat. or sat'd.) Saturated
SFC supercritical fluid chromatography
sgc Silica gel 60 chromatography
SiO₂ Silica gel
tBOC Cert-Butoxycarbonyl
t-Bu tert-butyl
TEA Triethylamine
Tf Trifluoromethane sulfonyl
TFA Trifluoroacetic acid
THE Tetrahydrofuran
TLC Thin layer chromatography
Ts Toluene sulfonyl
TsOH para-toluene sulfonic acid
TsOH.H₂O para-toluene sulfonic acid hydrate

### General Experimental Information

Unless otherwise noted, all reactions are magnetically stirred.

Unless otherwise noted, when ethyl acetate, hexanes, dichloromethane, 2-propanol, and methanol are used in the experiments described below, they are Fisher Optima grade solvents.

Unless otherwise noted, when diethyl ether is used in the experiments described below, it is Fisher ACS certified material and is stabilized with BHT.

Unless otherwise noted, “concentrated to dryness” means evaporating the solvent from a solution or mixture using a rotary evaporator.

Unless otherwise noted, flash chromatography is carried out on an Isco, Analogix, or Biotage automated chromatography system using a commercially available cartridge as the column. Columns may be purchased from Isco, Analogix, Biotage, Varian, or Supelco and are usually filled with silica gel as the stationary phase. Microwave chemistry is performed in sealed glass tubes in a Biotage microwave reactor.

### General Synthetic Schemes

A general procedure for the preparation of carboxylic acids xi is outlined in Scheme 9 below. Using a peptide coupling reagent such as PyBOP, HATU, EDCI/HOBt and the like, N—BOC glycine (i) can be coupled with amines such as ii to afford peptides iii. Removal of the Boc group can be accomplished using conditions such as TFA in CH₂Cl₂ to provide compound Iv. Reaction of compound Iv with a cyclic ketone represented by compound v under either basic or acidic conditions, using conventional or microwave heating will afford the spirocycle vi. Oxidation of vi to the imidazolone vii can be accomplished via a two-step chlorination/elimination approach. Further oxidation of vii to viii can be performed upon treatment of vii with m-CPBA. Compound ix, wherein X is triflyl can be accessed from compound viii upon treatment with trifluoromethanesulfonic anhydride and triethylamine. Conversely, compound ix, wherein X is chloro can be accessed from compound viii upon treatment with POCl₃ and iPr₂NEt in toluene at reflux. Compounds x, wherein G is attached to the imidazolone ring through a nitrogen, can be prepared via reaction of compounds ix with a primary or secondary, cyclic or acyclic amine in the presence of a base such as iPr₂NEt and the like in a solvent such as MeCN and the like under either conventional or microwave heating. Hydrolysis of the ester present in compound x with an aqueous solution of a base such as NaOH and the like in a solvent mixture such as MeOH/THF and the like will afford compound xi. Alternatively, the ester present in compound x may be cleaved with a reagent such as BBr₃ in a solvent such as CH₂Cl₂ and the like to provide compound xi.

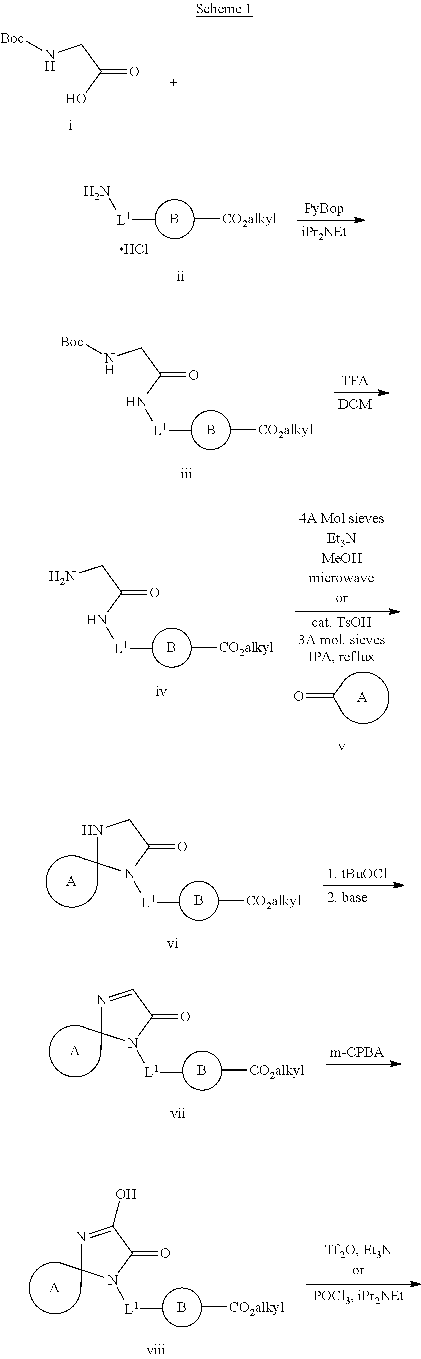

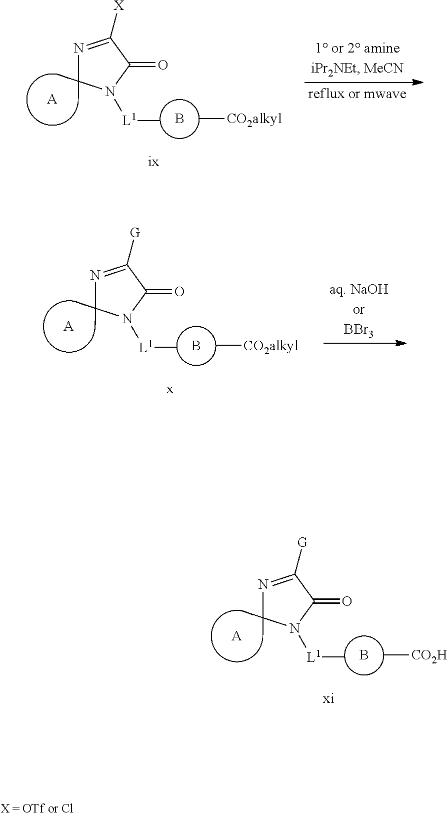

General experimental procedures for the synthesis of benzamides xiv and xvii from benzoic acid xi are described in Scheme 2 and Scheme 3 below.

Treatment of a suitable amine xiii or xv and a benzoic acid xi with a coupling reagent such as PyBOP and the like in a solvent such as DMF and the like will provide compounds xiii or xvi (Scheme 4 Cleavage of the tert-butyl ester present in compound xiii with an acid such as trifluoroacetic acid or hydrochloric and the like will afford compound xiv. Cleavage of the tert-butyl ester present in compound xvi with an acid such as trifluoroacetic acid or hydrochloric and the like will afford compound xvii.

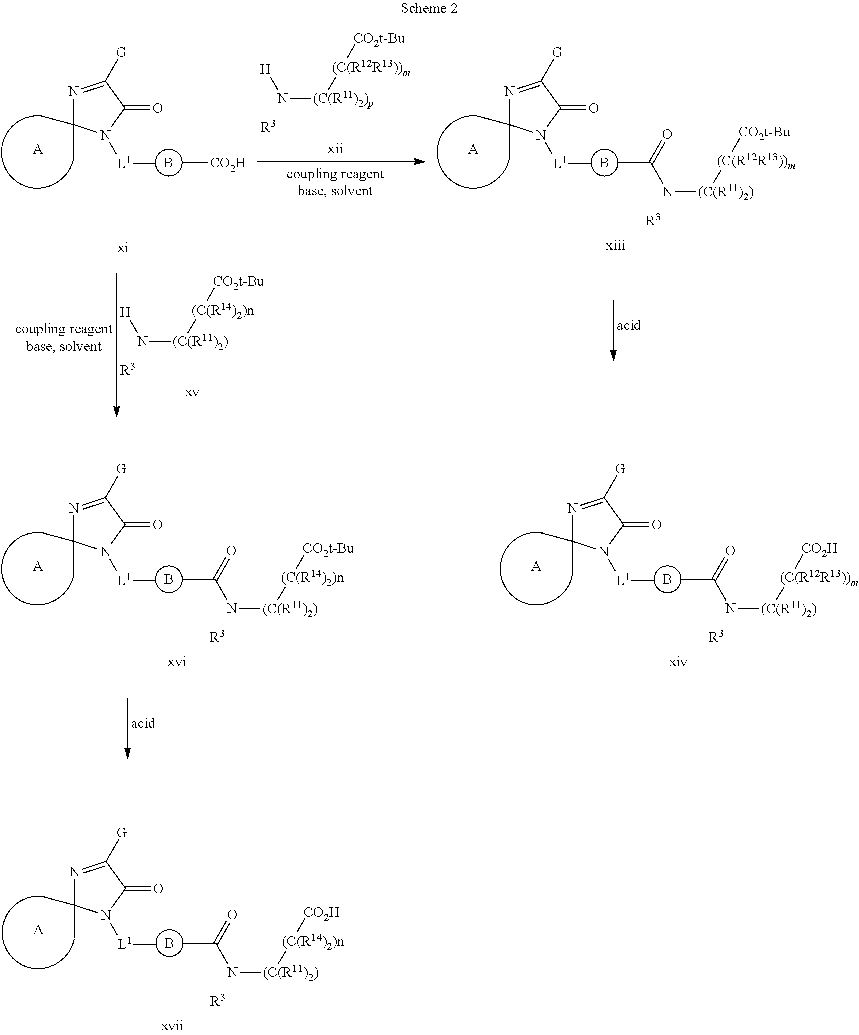

In Scheme 3, treatment of a suitable amine xviii or xix and a benzoic acid xi with a coupling reagent such as PyBOP and the like in a solvent such as DMF and the like will provide compounds xx or xxi. Hydrolysis of the methyl ester present in compound xx with an aqueous solution of a base such as NaOH and the like in a solvent mixture such as MeOH/THF and the like will afford compound xiv. Hydrolysis of the methyl ester present in compound xxi with an aqueous solution of a base such as NaOH and the like in a solvent mixture such as MeOH/THF and the like will afford compound xvii.

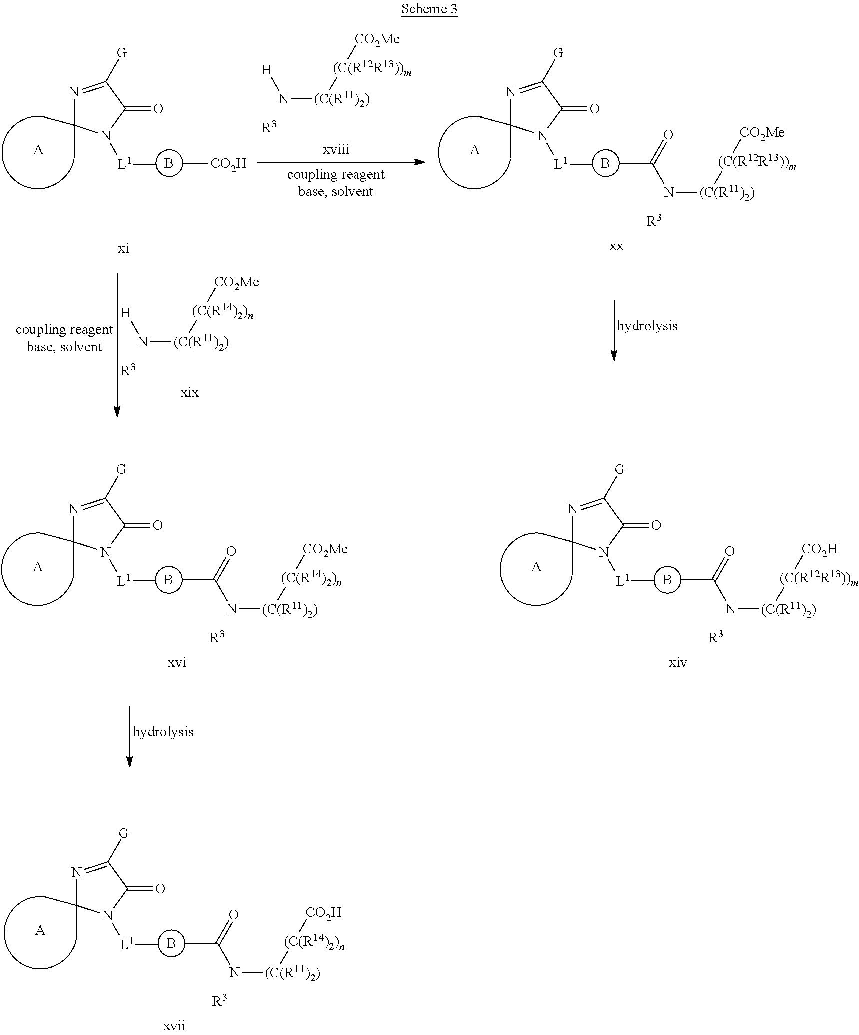

A general experimental procedure for the synthesis of benzamide xxiii from benzoic acid xi is described in Scheme 4 below. Treatment of xxii (in its free or acid salt form) and a benzoic acid xi with a coupling reagent such as PyBOP and the like and a base such as iPr₂NEt and the like in a solvent such as DMF and the like will provide a desired compound xxiii.

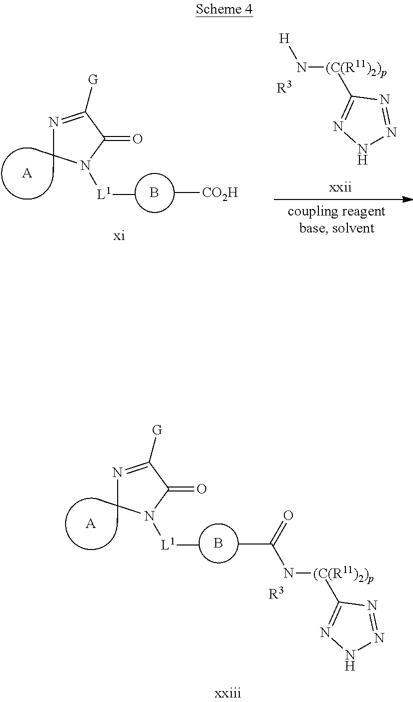

A general method for the synthesis of intermediates xi, wherein substituent G is alkyl, cycloalkyl, or cycloalkenyl is outlined in Scheme 5. The Boc-protected α-amino acid xxiv and the amine hydrochloride salt ii can be coupled using a reagent such as HATU and the like, with a base such as iPr₂NEt and the like in a suitable solvent such as DMF and the like to afford the peptide xxv. The Boc group present in xxv can be removed with an acid such as trifluoroacetic acid and the like to afford a compound such as xxvi. Spirocyclic compounds such as xxvii can be prepared from xxvi and a suitable ketone v under either base- or acid-catalyzed dehydrative cyclization. Oxidation to imidazolones x can be accomplished via a one-pot chlorination/elimination of compound xv. Hydrolysis of the ester present in compound x with an aqueous solution of a base such as NaOH and the like in a solvent mixture such as MeOWTHF and the like will afford compound xi.

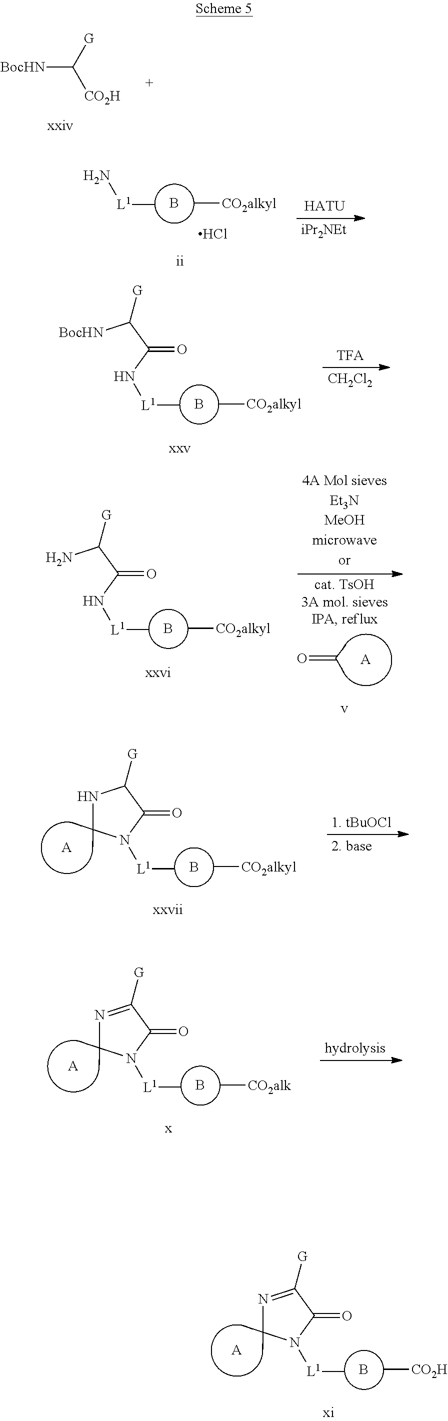

A general approach to enantiomerically enriched amines xxxiii and xxxiv is illustrated in Scheme 6. This approach is familiar to one skilled in the art, and numerous examples exist in the literature (for example see: Cogan, D.A.; Liu, G.; Ellman, J. A. Tetrahedron 1999, 55, 8883-8904). The condensation of the sulfinamide xxviii with aldehydes xix provides the imines xxx. Organometallic reagents (such as grignards: R^{5A}MgBr) add to imines xxx to provide diastereomeric mixtures of the sulfinamides xxxi and xxxii. These diastereomers can be purified by crystallization or chiral HPLC methods that are known to those skilled in the art. The pure diasteroemers xxxi and xxxii can be treated with HCl to provide the enantiomerically enriched amine HCl salts xxxiii and xxxiv, respectively.

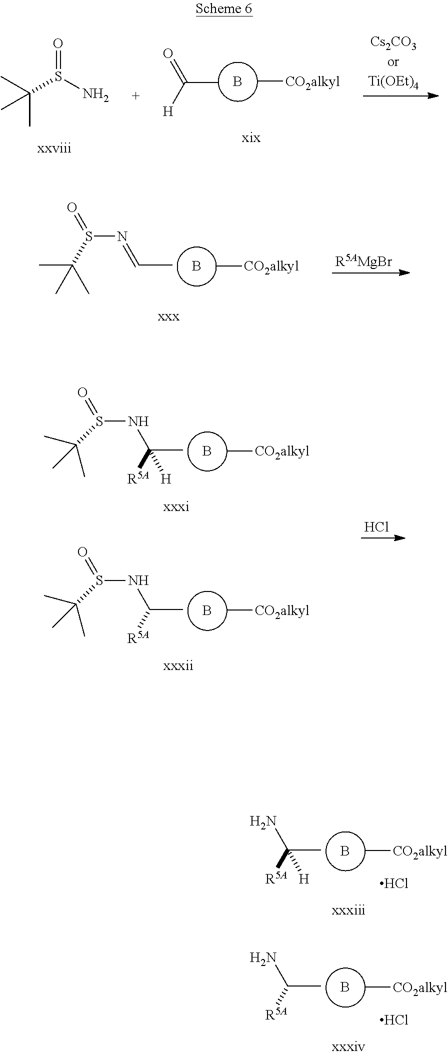

A related approach to these types of enantiomericaly enriched amine NCl salts is illustrated in Scheme 7. The condensation of the sulfinamide xxviii with ketones such as xxxv will provide ketimines xxxvi. Imines such as xxxvi can be reduced (see Tanuwidjaja, J.; Peltier, H. M.; Ellman, J. A. J. Org. Chem. 2007, 72, 626) with various reducing reagents to provide sulfinamides such as xxxi and xxxii. As previously described, these sulfinamides can be treated with HCl to provide the enantiomerically enriched amine NCl salts xxxiii and xxxiv.

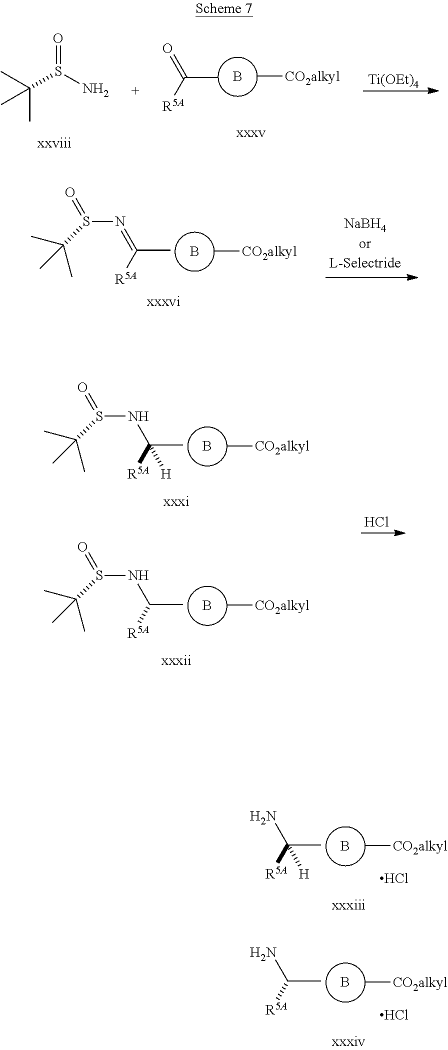

A general approach for the synthesis styrenyl imidazolones such as compound xxxix is summarized in Scheme 8 below. The previously described compound vi can be treated with m-CPBA in a solvent such as dichloromethane and the like to afford the nitrone xxxvii. The nitrone can then undergo a [3+2] cycloaddition with a styrene substituted with any of the substituents described in Formula A, items (i)-(xiii), as described for substituent G. This will provide the substituted phenyl isoxazolidine xxxviii. Treatment of xxxviii with aqueous NaOH followed by aqueous NCl will result in the formation of the styrenyl compounds xxxix.

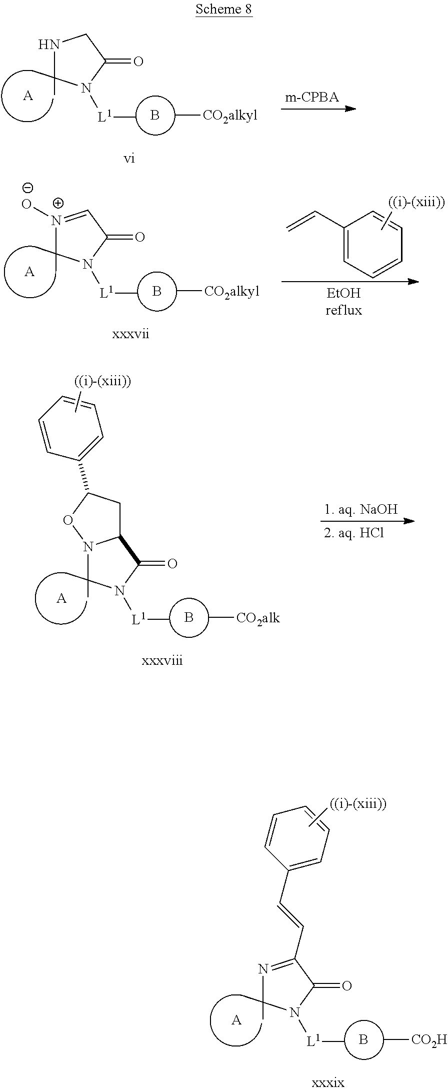

Also known to those skilled in the art, are the formation of tetrazole terminated compounds of the formula xxiii via the method outlined in Scheme 9. The coupling of acids xi with cyano-substituted alkyl amines xl produces cyanoalkyl-amides of the type xli. The cyano group in xli will react with various reagents, including sodium azide in the presence of an alkyl amine hydrochloride, or sodium azide in the presence of ZnBr₂ in isopropanol/water to provide compounds xxiii.

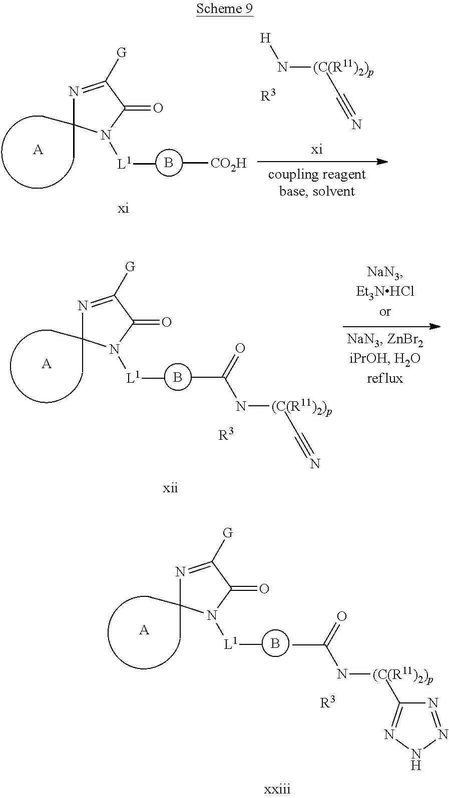

In an alternative method described in Scheme 10, nitrones such as xxxvii can be treated with a reagent such as POCl₃ and the like in the presence of a base such as iPr₂NEt and the like in a solvent such as toluene and the like to afford the chloroimidazolone xlii. Treatment of xlii with a primary or secondary amine at temperatures ranging from room temperature to 150° C. under either conventional or microwave heating will afford compounds x, wherein G is an amine linked to the core through nitrogen.

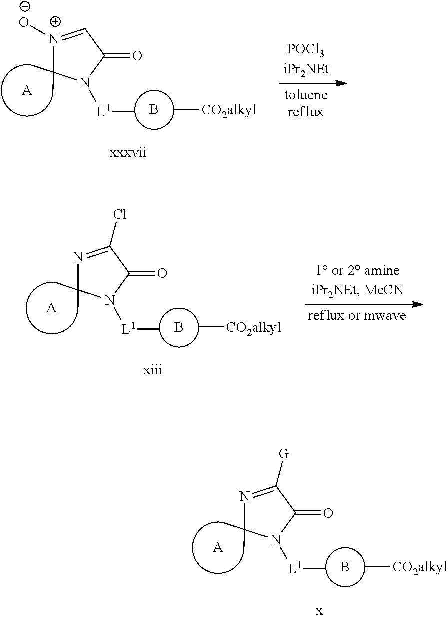

Alternatively, as described in Scheme 11, one can treat an intermediate such as viii with a coupling reagent such as PyBOP, PyBroP, or BOP-Cland the like in the presence of a primary or secondary amine, and a base such as iPr₂NEt and the like in a solvent such as MeCN or 1,4-dioxane and the like to directly prepare compounds x, wherein G is an amine linked to the core through nitrogen.

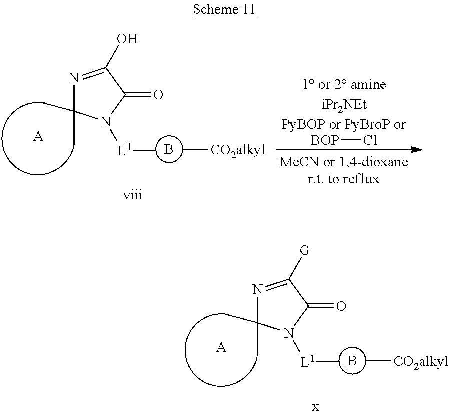

### PROCEDURES/EXAMPLES

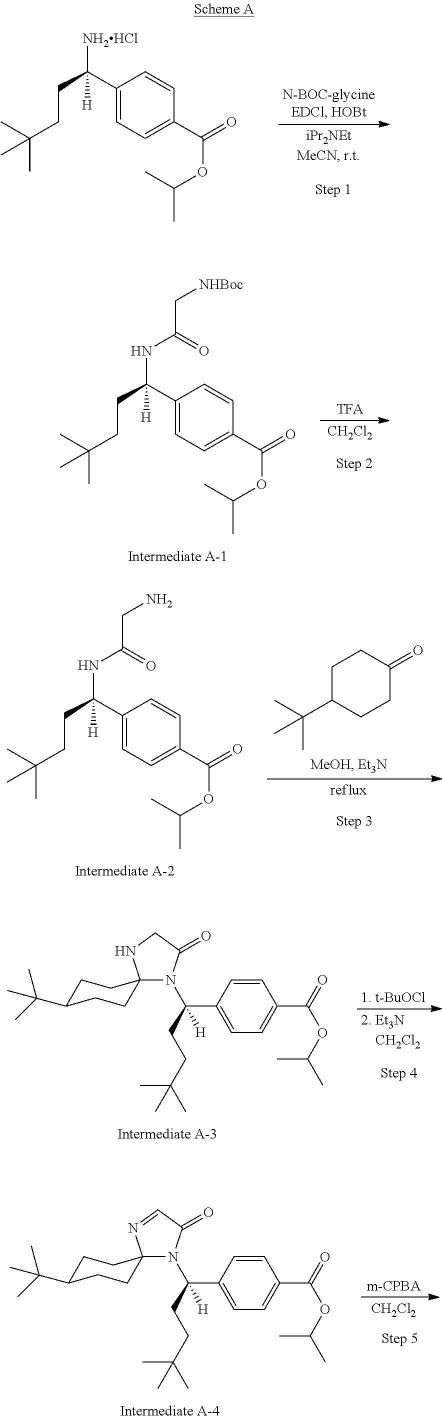

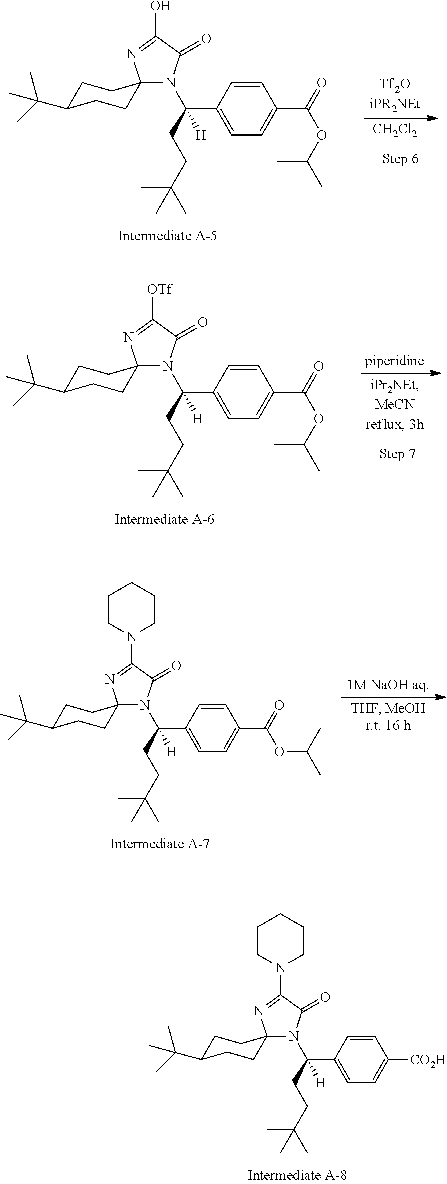

### Step 1

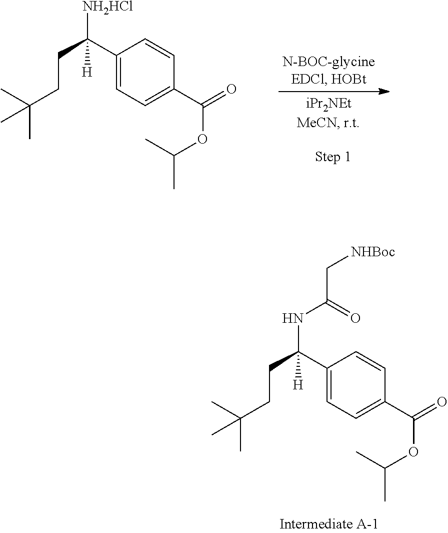

A solution of N—BOC-glycine (6.13 g, 35.0 mmol, 1.10 eq), HOBt (2.68 g, 17.5 mmol, 0.55 eq), and iPr₂NEt (18.3 mL, 105 mmol, 3.29 eq) in MeCN (100 mL) at 0° C. was treated with EDCI (6.71 g, 35.0 mmol, 1.10 eq) followed by the amine hydrochloride salt (10.00 g, 31.9 mmol, 1.00 eq). The resulting mixture was stirred at 0° C. for 15 minutes. The reaction was allowed to warm to room temperature and was stirred 16 h. The reaction was partitioned between EtOAc and a mixture of 1N HCl_{(aq.)} and brine. The aqueous layer was discarded and the organic layer was washed successively with saturated NaHCO_{3(aq.)} and brine, was dried over anhydrous sodium sulfate, filtered and evaporated to afford Intermediate A-1 (14.1 g, quant.) which was used in the next step without further purification.

### Step 2

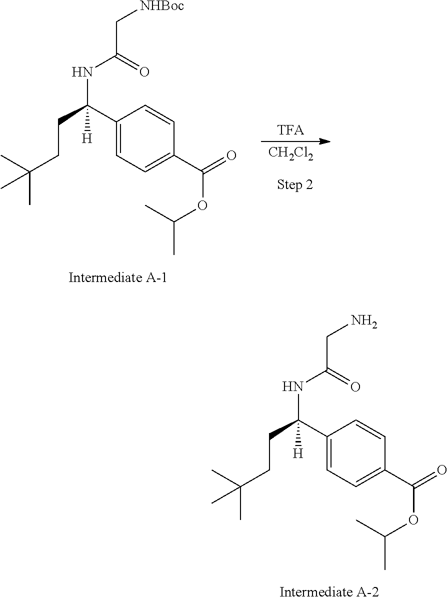

Intermediate A-1 (14.1 g, 32.4 mmol, 1 eq) was dissolved in CH₂Cl₂ (200 mL) and treated with TFA (20 mL). After 2 hours, TLC showed the reaction to be incomplete. An additional amount of TFA (20 mL) was added and the reaction was stirred for 2 hours more, at which point, the voltiles were removed in vacuo to afford an oily residue. The crude residue was partitioned between CH₂Cl₂ and 1M NaOH_{(aq.)}. The organic layer was saved and the aqueous layer was extracted with CH₂Cl₂. The organic layers were combined, washed with brine, dried over anhydrous sodium sulfate, filtered, and evaporated to afford Intermediate A-2 (10.51 g, 97%), which was used in the next step without further purification.

### Step 3

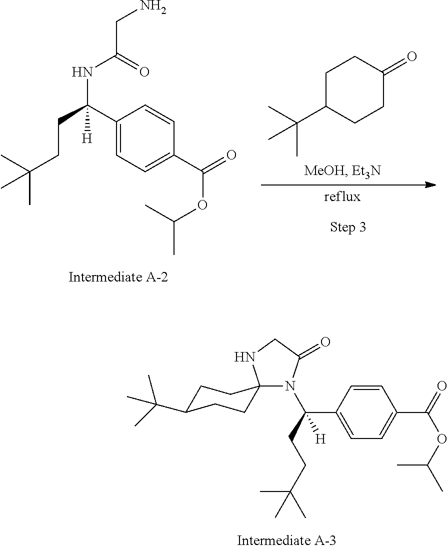

A solution of Intermediate A-2 (2.63 g, 7.86 mmol, 1.00 eq), 4-tert-butylcyclohexanone (3.63 g, 23.5 mmol, 2.99 eq), and triethylamine (5.90 mL, 42.3 mmol, 5.38 eq) in MeOH (45 mL) in a round bottomed flask was charged with powdered, 4 angstrom molecular sieves (3.6 g, dried under vacuum, 72 hours at 130° C.). A reflux condenser and nitrogen line were attached and the mixture was refluxed 24 h. The reaction was cooled to room temperature and filtered through Celite®. The Celite® pad was washed with MeOH. The filtrates were combined and concentrated to afford a residue which was purified via silica gel chromatography (gradient elution, 0% to 100% EtOAc in hexanes, SiO₂) to afford Intermediate A-3 (1.78 g, 48%) as a viscous oil.

### Step 4

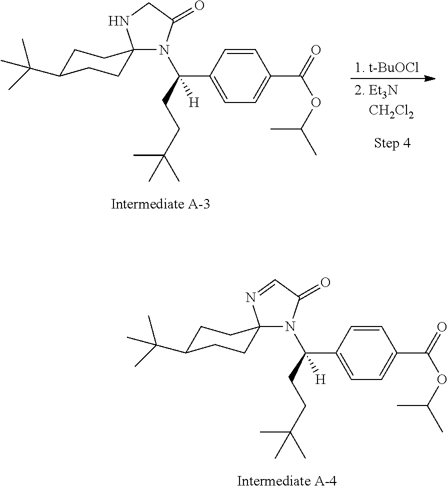

A solution of Intermediate A-3 (1.00 g, 2.12 mmol, 1.00 eq) in CH₂Cl₂ (30 mL) at room temperature was treated with Pert-butyl hypochlorite (0.29 mL, 2.55 mmol, 1.20 eq). After stirring for 45 minutes, triethylamine (1.2 mL, 8.50 mmol, 4.00 eq) was added dropwise, and the resulting solution was stirred for 45 minutes more. The reaction was quenched by adding 10% sodium bisulfite_{(aq.)} while stirring. The organic layer was removed and saved, and the aqueous layer was extracted with CH₂Cl₂. The organic layers were combined, washed with brine, dried over anhydrous sodium sulfate, filtered, and evaporated to afford a crude residue which was purified via silica gel chromatography (gradient elution, 0% to 30% EtOAc in hexanes, SiO₂) to afford Intermediate A-4 (730 mg, 73%) as a white foam.

### Step 5

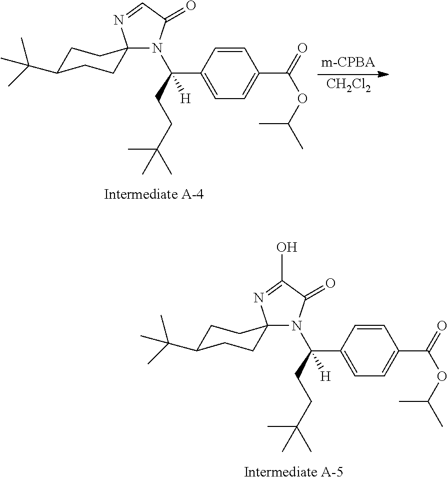

Intermediate A-4 (730 mg, 1.6 mmol, 1.0 eq) was dissolved in CH₂Cl₂ (10 and treated with m-CPBA (77% w/w with water, 1.05 g, 4.67 mmol, 3.00 eq) and stirred at room temperature overnight. The reaction was quenched with 10% sodium thiosulfate_{(aq.)} and saturated NaHCO_{3(aq.)}. The resulting biphasic mixture was stirred until both layers were clear. The layers were separated and both were saved. The aqueous layer was extracted with CH₂Cl₂. The combined organic layers were washed with brine, dried over anhydrous sodium sulfate, filtered, and evaporated to afford a crude product which was purified via silica gel chromatography (gradient elution, 0% to 100% EtOAc in hexanes, SiO₂) to afford Intermediate A-5 (560 mg, 74%) as a white foam.

### Step 6

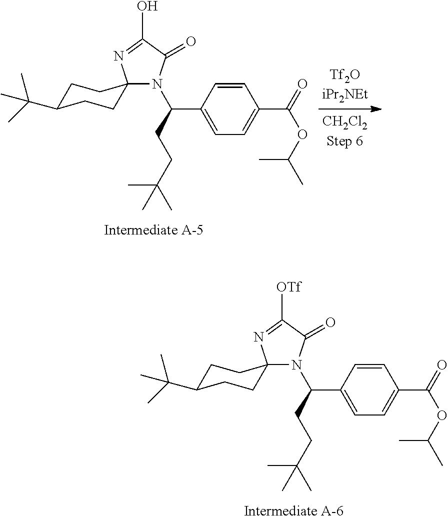

Intermediate A-5 (560 mg, 1.16 mmol, 1.00 eq) and iPr₂NEt (0.50 mL, 2.89 mmol, 2.5 eq) were dissolved in CH₂Cl₂ (30 mL) and cooled to −10° C. Trifluoromethanesulfonic anhydride (0.233 mL, 1.39 mmol, 1.20 eq) was added dropwise and the mixture was stirred for 30 minutes at −10° C. An additional amount of trifluoromethanesulfonic anhydride (0.2 mL) was added and the reaction was stirred for an additional 30 minutes. An additional amount of iPr₂NEt (1.0 mL, 5.78 mmol, 5 eq) was added and the reaction was stirred for 5 minutes. The reaction mixture was partitioned between CH₂Cl₂ and brine. The layers were separated and both were saved. The aqueous layer was extracted with CH₂Cl₂. The combined organic layers were dried over anhydrous sodium sulfate, filtered, and evaporated to afford a crude product which was purified via silica gel chromatography (gradient elution, 0% to 20% EtOAc in hexanes, SiO₂) to afford Intermediate A-6 (478 mg, 67%).

### Step 7

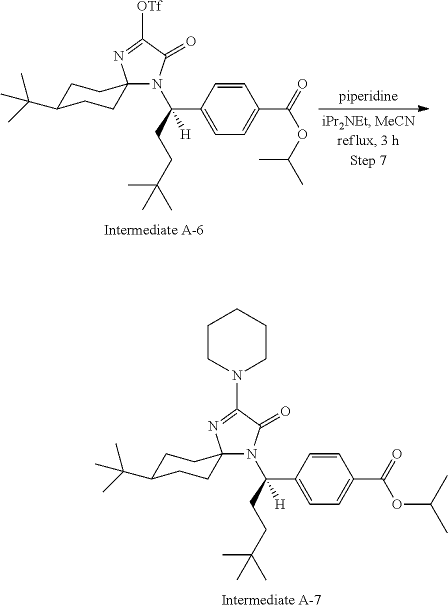

Intermediate A6 (200 mg, 0.32 mmol, 1 eq), piperidine (0.096 mL, 0.973 mmol, 3 eq), and iPr₂NEt (0.17 mL, 0.973 mmol, 3 eq) were dissolved in MeCN (4 mL), and were heated at reflux for 3 h. The reaction mixture was cooled to room temperature, and was concentrated. The residue was partitioned between EtOAc and 1N HCl_{(aq.)}. After discarding the aqueous layer, the organic layer was washed with brine, dried over anhydrous Na₂SO₄, filtered, and evaporated to afford a crude residue. Silica gel chromatography (gradient elution, 0% to 100% EtOAc in hexanes) afforded Intermediate A-7 (37 mg, 21%) as a clear colorless film.

### Step 8

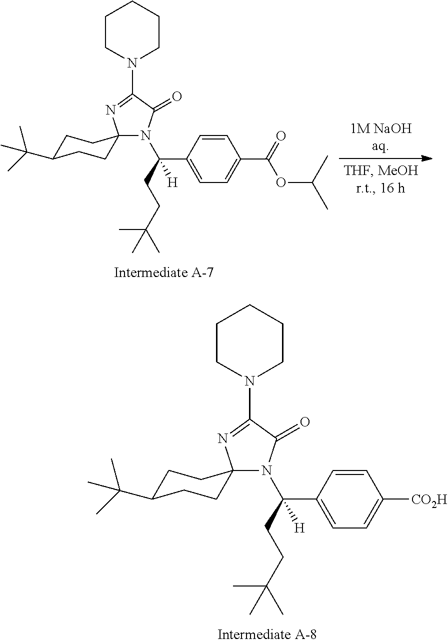

Intermediate A-7 (37 mg, 0.067 mmol) was dissolved in MeOH (6 mL) and THF (6 mL). Addition of 1M NaOH_{(aq.)} (1.5 mL) was followed by stirring overnight at room temperature. The reaction was partitioned between EtOAc and 1N HCl_{(aq.)}. The aqueous layer was discarded and the organic layer was washed with brine, dried over anhydrous Na₂SO₄, filtered, and evaporated to afford Intermediate A-8 (34 mg, 99%) which was used in the next step without further purification.

TABLE 1
Using the requisite starting materials, and a method similar to that outlined in Scheme A, the following compounds were prepared:

| | Ketone | Amine | Inter- | |
|---|---|---|---|---|
| Amine Used | Used in | Used in | mediate | |
| in Step 1 | Step 3 | Step 7 | Number | Intermediate Prepared |
| 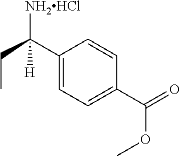 | 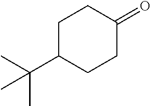 | 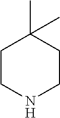 | A-9 | 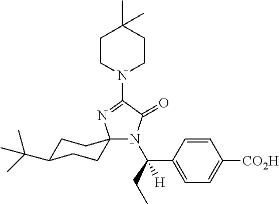 [SMILES: CC[C@H](c1ccc(C(=O)O)cc1)N1C(=O)C(N2CCC(C)(C)CC2)=NC12CCC(C(C)(C)C)CC2] |
| 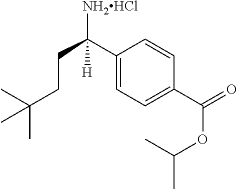 | 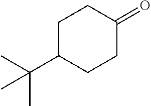 | 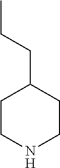 | A-10 | 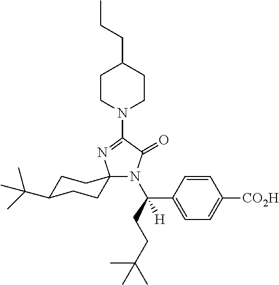 [SMILES: CCCC1CCN(C2=NC3(CCC(C(C)(C)C)CC3)N([C@H](CCC(C)(C)C)c3ccc(C(=O)O)cc3)C2=O)CC1] |
| 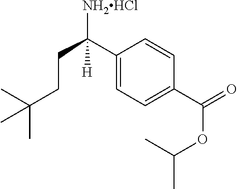 | 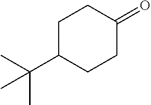 | 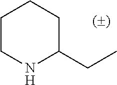 | A-11 | 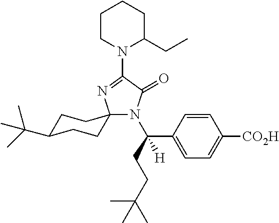 [SMILES: CCC1CCCCN1C1=NC2(CCC(C(C)(C)C)CC2)N([C@H](CCC(C)(C)C)c2ccc(C(=O)O)cc2)C1=O] mixture of diastereomers |
| 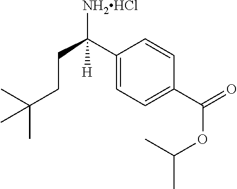 | 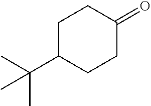 | 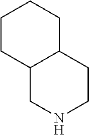 mixture of isomers | A-12 | 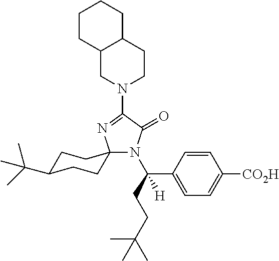 [SMILES: CC(C)(C)CC[C@H](c1ccc(C(=O)O)cc1)N1C(=O)C(N2CCC3CCCCC3C2)=NC12CCC(C(C)(C)C)CC2] mixture of isomers |
| 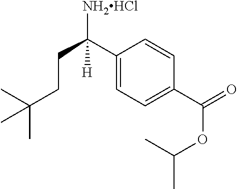 | 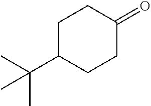 | 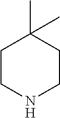 | A-13 | 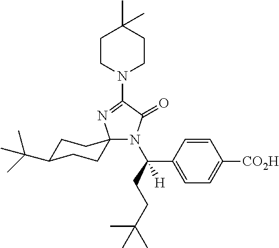 [SMILES: CC(C)(C)CC[C@H](c1ccc(C(=O)O)cc1)N1C(=O)C(N2CCC(C)(C)CC2)=NC12CCC(C(C)(C)C)CC2] |
| 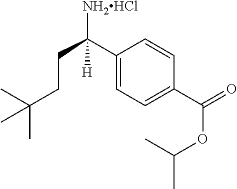 | 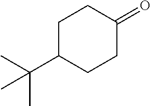 | 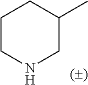 | A-14 | 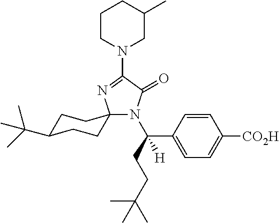 [SMILES: CC1CCCN(C2=NC3(CCC(C(C)(C)C)CC3)N([C@H](CCC(C)(C)C)c3ccc(C(=O)O)cc3)C2=O)C1] mixture of diastereomers |
| 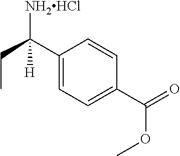 | 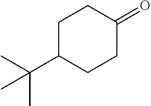 | 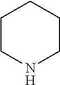 | A-15 | 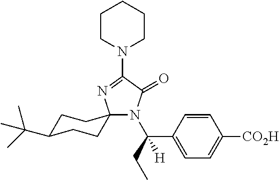 [SMILES: CC[C@H](c1ccc(C(=O)O)cc1)N1C(=O)C(N2CCCCC2)=NC12CCC(C(C)(C)C)CC2] |
| 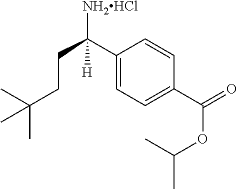 | 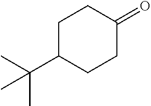 | 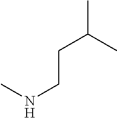 | A-16 | 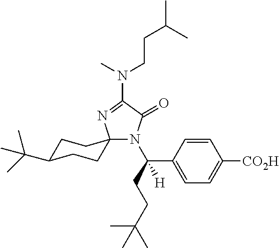 [SMILES: CC(C)CCN(C)C1=NC2(CCC(C(C)(C)C)CC2)N([C@H](CCC(C)(C)C)c2ccc(C(=O)O)cc2)C1=O] |
| 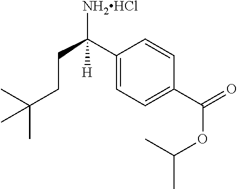 | 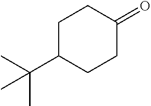 | 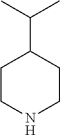 | A-17 | 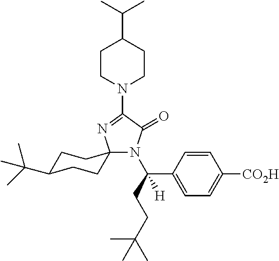 [SMILES: CC(C)C1CCN(C2=NC3(CCC(C(C)(C)C)CC3)N([C@H](CCC(C)(C)C)c3ccc(C(=O)O)cc3)C2=O)CC1] |
| 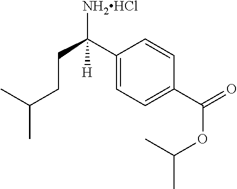 | 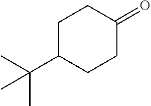 | 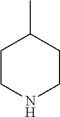 | A-18 | 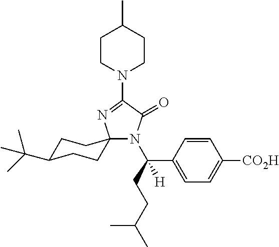 [SMILES: CC(C)CC[C@H](c1ccc(C(=O)O)cc1)N1C(=O)C(N2CCC(C)CC2)=NC12CCC(C(C)(C)C)CC2] |
| 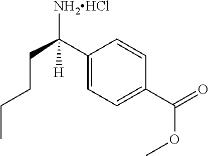 | 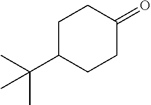 | 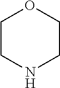 | A-19 | 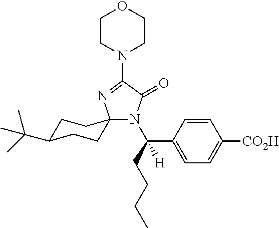 [SMILES: CCCC[C@H](c1ccc(C(=O)O)cc1)N1C(=O)C(N2CCOCC2)=NC12CCC(C(C)(C)C)CC2] |
| 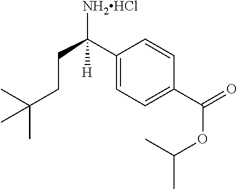 | 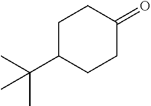 | 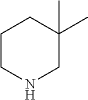 | A-20 | 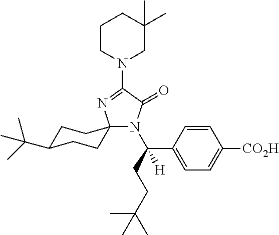 [SMILES: CC(C)(C)CC[C@H](c1ccc(C(=O)O)cc1)N1C(=O)C(N2CCCC(C)(C)C2)=NC12CCC(C(C)(C)C)CC2] |
| 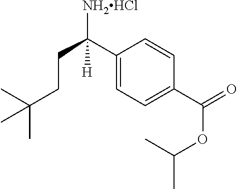 | 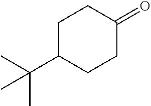 | 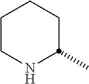 | A-21 | 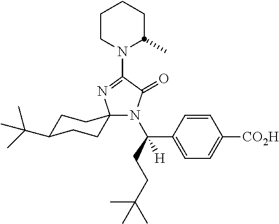 [SMILES: C[C@H]1CCCCN1C1=NC2(CCC(C(C)(C)C)CC2)N([C@H](CCC(C)(C)C)c2ccc(C(=O)O)cc2)C1=O] |
| 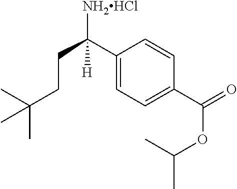 | 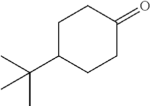 | 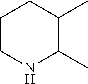 Intermediate L-1 (mixture of isomers) | A-22 | 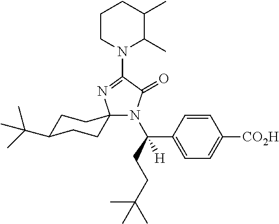 [SMILES: CC1CCCN(C2=NC3(CCC(C(C)(C)C)CC3)N([C@H](CCC(C)(C)C)c3ccc(C(=O)O)cc3)C2=O)C1C] (mixture of isomers) |
| 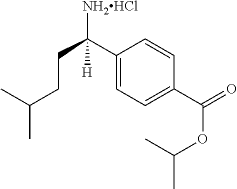 | 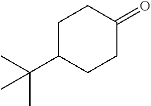 | 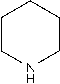 | A-23 | 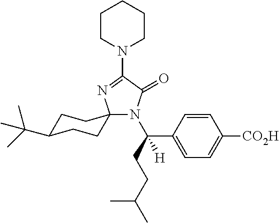 [SMILES: CC(C)CC[C@H](c1ccc(C(=O)O)cc1)N1C(=O)C(N2CCCCC2)=NC12CCC(C(C)(C)C)CC2] |
| 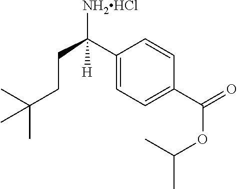 | 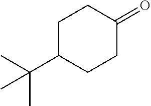 | 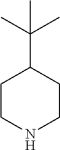 | A-24 | 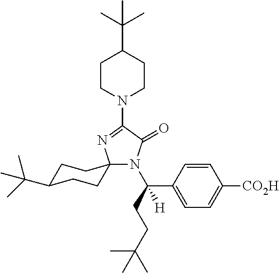 [SMILES: CC(C)(C)CC[C@H](c1ccc(C(=O)O)cc1)N1C(=O)C(N2CCC(C(C)(C)C)CC2)=NC12CCC(C(C)(C)C)CC2] |
| 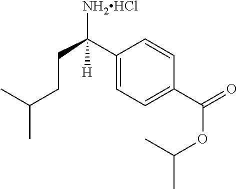 | 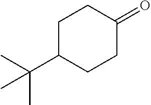 | 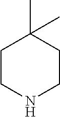 | A-25 | 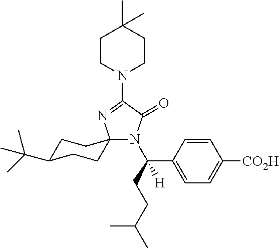 [SMILES: CC(C)CC[C@H](c1ccc(C(=O)O)cc1)N1C(=O)C(N2CCC(C)(C)CC2)=NC12CCC(C(C)(C)C)CC2] |
| 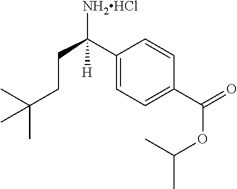 | 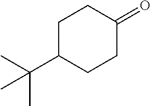 | 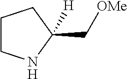 | A-26 | 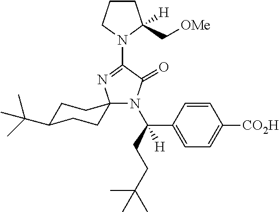 [SMILES: COC[C@@H]1CCCN1C1=NC2(CCC(C(C)(C)C)CC2)N([C@H](CCC(C)(C)C)c2ccc(C(=O)O)cc2)C1=O] |
| 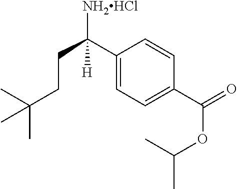 | 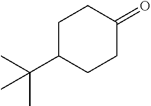 | 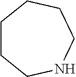 | A-27 | 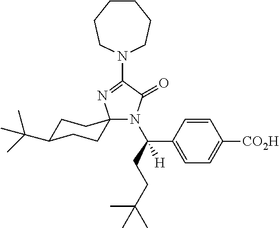 [SMILES: CC(C)(C)CC[C@H](c1ccc(C(=O)O)cc1)N1C(=O)C(N2CCCCCC2)=NC12CCC(C(C)(C)C)CC2] |
| 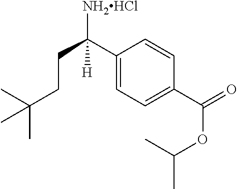 | 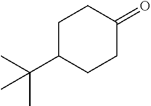 | 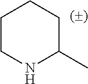 | A-28 | 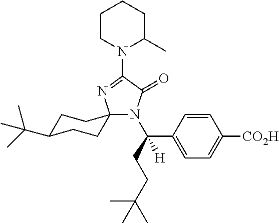 [SMILES: CC1CCCCN1C1=NC2(CCC(C(C)(C)C)CC2)N([C@H](CCC(C)(C)C)c2ccc(C(=O)O)cc2)C1=O] mixture of diastereomers |
| 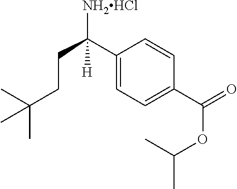 | 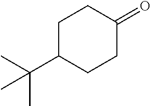 | 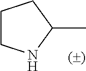 | A-29 | 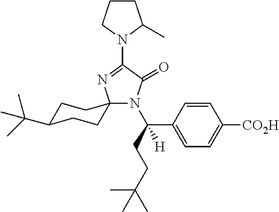 [SMILES: CC1CCCN1C1=NC2(CCC(C(C)(C)C)CC2)N([C@H](CCC(C)(C)C)c2ccc(C(=O)O)cc2)C1=O] mixture of diastereomers |
| 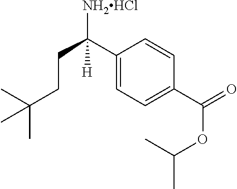 | 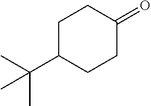 | 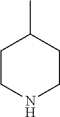 | A-30 | 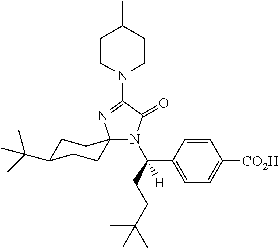 [SMILES: CC1CCN(C2=NC3(CCC(C(C)(C)C)CC3)N([C@H](CCC(C)(C)C)c3ccc(C(=O)O)cc3)C2=O)CC1] |
| 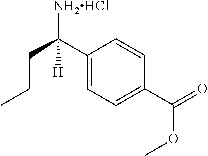 | 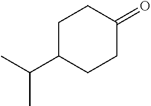 | 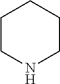 | A-31 | 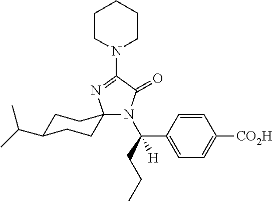 [SMILES: CCC[C@H](c1ccc(C(=O)O)cc1)N1C(=O)C(N2CCCCC2)=NC12CCC(C(C)C)CC2] |
| 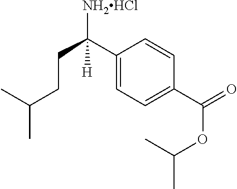 | 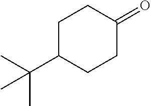 | 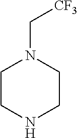 [SMILES: FC(F)(F)CN1CCNCC1] | A-32 | 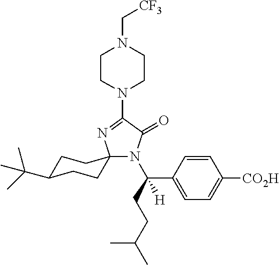 [SMILES: CC(C)CC[C@H](c1ccc(C(=O)O)cc1)N1C(=O)C(N2CCN(CC(F)(F)F)CC2)=NC12CCC(C(C)(C)C)CC2] |
| 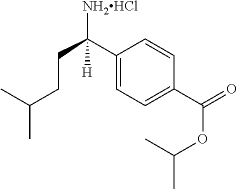 | 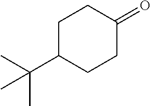 | 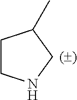 | A-33 | 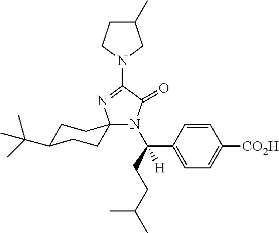 [SMILES: CC(C)CC[C@H](c1ccc(C(=O)O)cc1)N1C(=O)C(N2CCC(C)C2)=NC12CCC(C(C)(C)C)CC2] mixture of diastereomers |
| 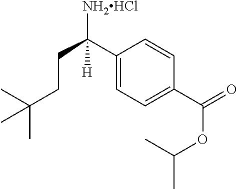 | 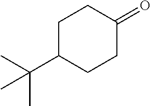 | 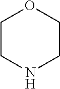 | A-34 | 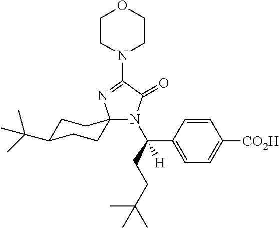 [SMILES: CC(C)(C)CC[C@H](c1ccc(C(=O)O)cc1)N1C(=O)C(N2CCOCC2)=NC12CCC(C(C)(C)C)CC2] |
| 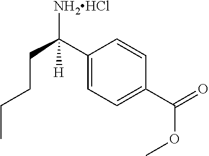 | 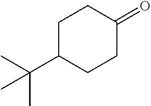 | 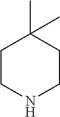 | A-35 | 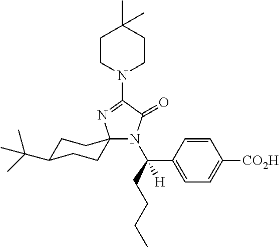 [SMILES: CCCC[C@H](c1ccc(C(=O)O)cc1)N1C(=O)C(N2CCC(C)(C)CC2)=NC12CCC(C(C)(C)C)CC2] |
| 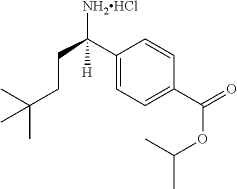 | 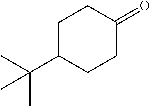 | 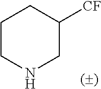 | A-36 | 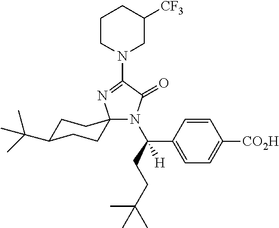 [SMILES: CC(C)(C)CC[C@H](c1ccc(C(=O)O)cc1)N1C(=O)C(N2CCCC(C(F)(F)F)C2)=NC12CCC(C(C)(C)C)CC2] mixture of diastereomers |
| 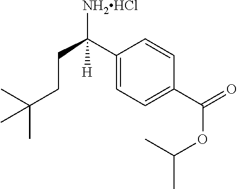 | 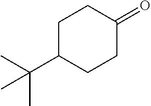 | 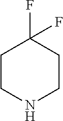 | A-37 | 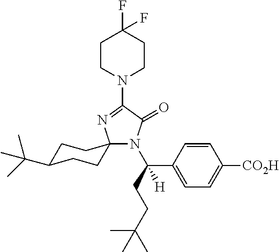 [SMILES: CC(C)(C)CC[C@H](c1ccc(C(=O)O)cc1)N1C(=O)C(N2CCC(F)(F)CC2)=NC12CCC(C(C)(C)C)CC2] |
| 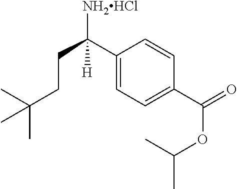 | 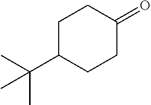 | 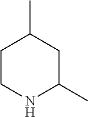 Intermediate L-2 (mixture of isomers) | A-38 | 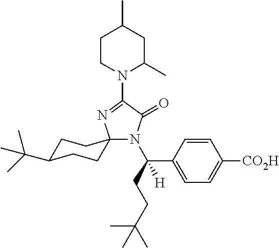 [SMILES: CC1CCN(C2=NC3(CCC(C(C)(C)C)CC3)N([C@H](CCC(C)(C)C)c3ccc(C(=O)O)cc3)C2=O)C(C)C1] (mixture of isomers) |
| 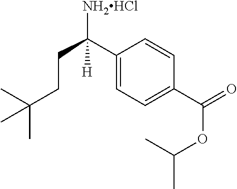 | 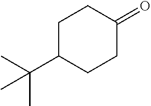 | 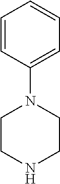 [SMILES: c1ccc(N2CCNCC2)cc1] | A-39 | 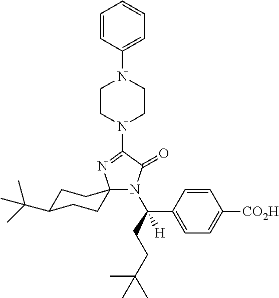 [SMILES: CC(C)(C)CC[C@H](c1ccc(C(=O)O)cc1)N1C(=O)C(N2CCN(c3ccccc3)CC2)=NC12CCC(C(C)(C)C)CC2] |
| 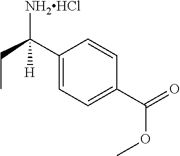 | 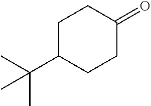 | 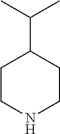 | A-40 | 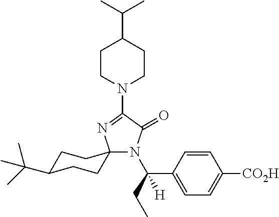 [SMILES: CC[C@H](c1ccc(C(=O)O)cc1)N1C(=O)C(N2CCC(C(C)C)CC2)=NC12CCC(C(C)(C)C)CC2] |
| 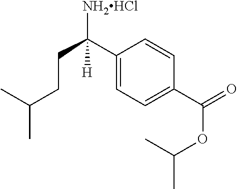 | 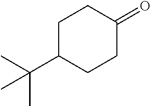 | 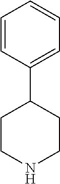 [SMILES: c1ccc(C2CCNCC2)cc1] | A-41 | 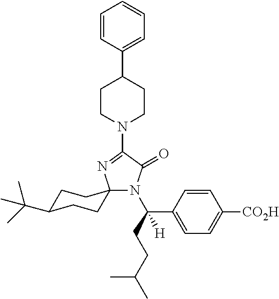 [SMILES: CC(C)CC[C@H](c1ccc(C(=O)O)cc1)N1C(=O)C(N2CCC(c3ccccc3)CC2)=NC12CCC(C(C)(C)C)CC2] |
| 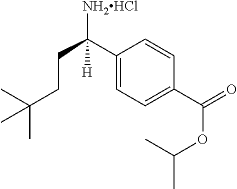 | 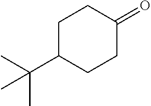 | 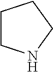 | A-42 | 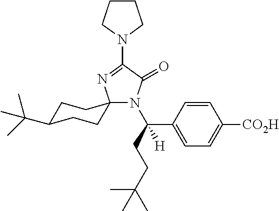 [SMILES: CC(C)(C)CC[C@H](c1ccc(C(=O)O)cc1)N1C(=O)C(N2CCCC2)=NC12CCC(C(C)(C)C)CC2] |
| 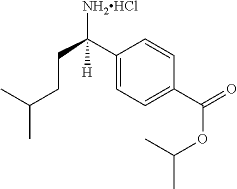 | 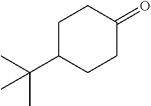 | 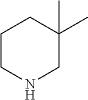 | A-43 | 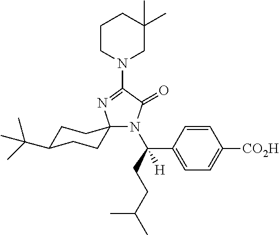 [SMILES: CC(C)CC[C@H](c1ccc(C(=O)O)cc1)N1C(=O)C(N2CCCC(C)(C)C2)=NC12CCC(C(C)(C)C)CC2] |
| 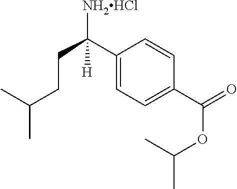 | 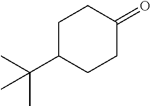 | 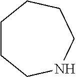 | A-44 | 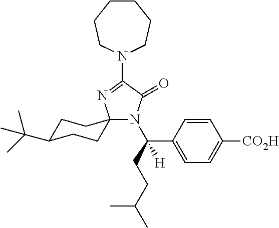 [SMILES: CC(C)CC[C@H](c1ccc(C(=O)O)cc1)N1C(=O)C(N2CCCCCC2)=NC12CCC(C(C)(C)C)CC2] |
| 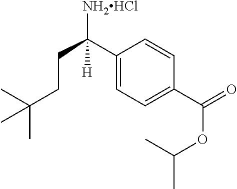 | 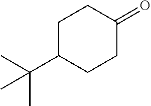 | 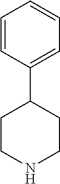 [SMILES: c1ccc(C2CCNCC2)cc1] | A-45 | 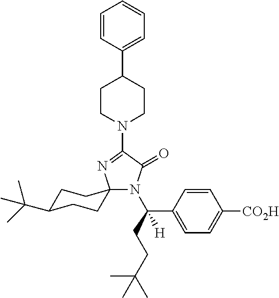 [SMILES: CC(C)(C)CC[C@H](c1ccc(C(=O)O)cc1)N1C(=O)C(N2CCC(c3ccccc3)CC2)=NC12CCC(C(C)(C)C)CC2] |
| 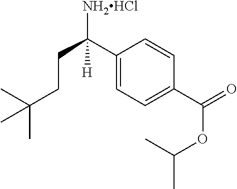 | 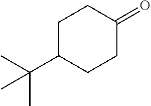 | 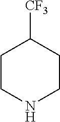 | A-46 | 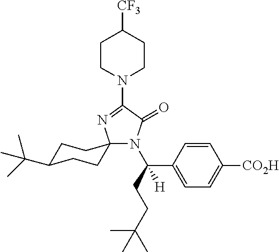 [SMILES: CC(C)(C)CC[C@H](c1ccc(C(=O)O)cc1)N1C(=O)C(N2CCC(C(F)(F)F)CC2)=NC12CCC(C(C)(C)C)CC2] |
| 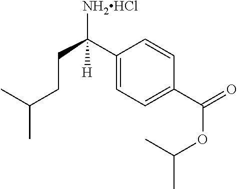 | 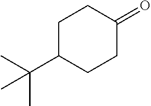 | 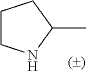 | A-47 | 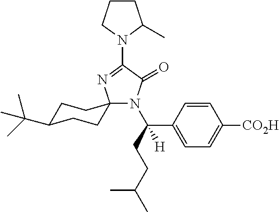 [SMILES: CC(C)CC[C@H](c1ccc(C(=O)O)cc1)N1C(=O)C(N2CCCC2C)=NC12CCC(C(C)(C)C)CC2] mixture of diastereomers |
| 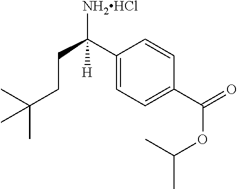 | 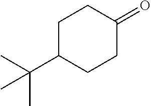 | 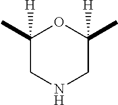 | A-48 | 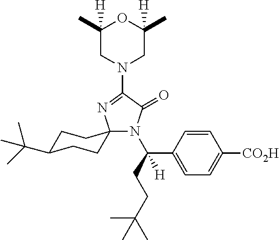 [SMILES: C[C@@H]1CN(C2=NC3(CCC(C(C)(C)C)CC3)N([C@H](CCC(C)(C)C)c3ccc(C(=O)O)cc3)C2=O)C[C@H](C)O1] |
| 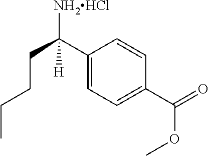 | 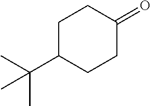 | 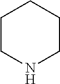 | A-49 | 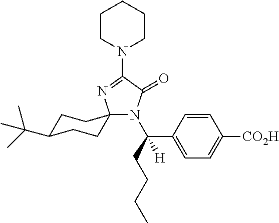 [SMILES: CCCC[C@H](c1ccc(C(=O)O)cc1)N1C(=O)C(N2CCCCC2)=NC12CCC(C(C)(C)C)CC2] |
| 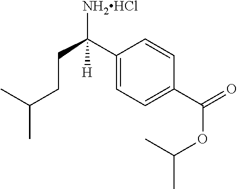 | 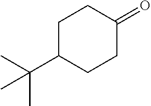 | 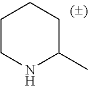 | A-50 | 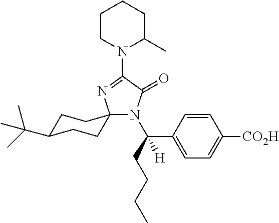 [SMILES: CCCC[C@H](c1ccc(C(=O)O)cc1)N1C(=O)C(N2CCCCC2C)=NC12CCC(C(C)(C)C)CC2] mixture of diastereomers |
| 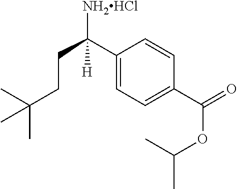 | 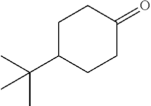 | 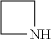 | A-51 | 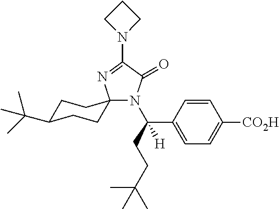 [SMILES: CC(C)(C)CC[C@H](c1ccc(C(=O)O)cc1)N1C(=O)C(N2CCC2)=NC12CCC(C(C)(C)C)CC2] |
| 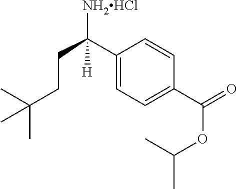 | 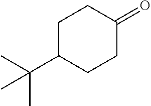 | 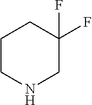 | A-52 | 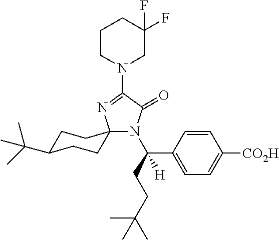 [SMILES: CC(C)(C)CC[C@H](c1ccc(C(=O)O)cc1)N1C(=O)C(N2CCCC(F)(F)C2)=NC12CCC(C(C)(C)C)CC2] |
| 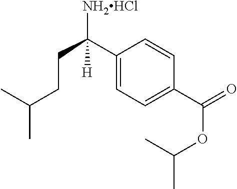 | 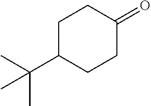 | 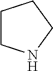 | A-53 | 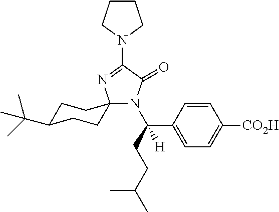 [SMILES: CC(C)CC[C@H](c1ccc(C(=O)O)cc1)N1C(=O)C(N2CCCC2)=NC12CCC(C(C)(C)C)CC2] |
| 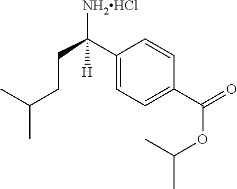 | 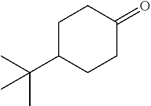 | 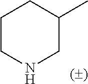 | A-54 | 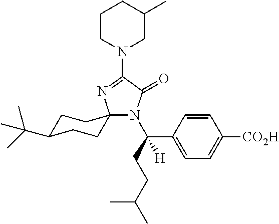 [SMILES: CC(C)CC[C@H](c1ccc(C(=O)O)cc1)N1C(=O)C(N2CCCC(C)C2)=NC12CCC(C(C)(C)C)CC2] mixture of diastereomers |
| 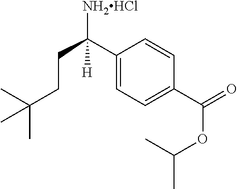 | 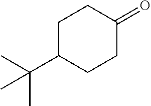 | 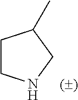 | A-55 | 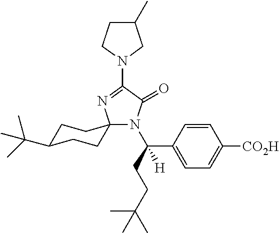 [SMILES: CC1CCN(C2=NC3(CCC(C(C)(C)C)CC3)N([C@H](CCC(C)(C)C)c3ccc(C(=O)O)cc3)C2=O)C1] mixture of diastereomers |
| 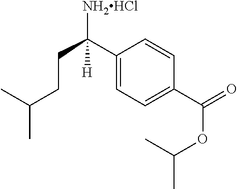 | 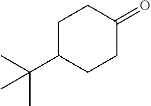 | 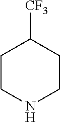 | A-56 | 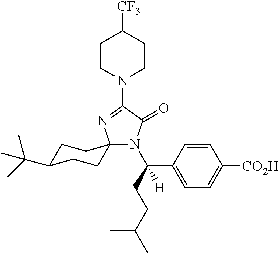 [SMILES: CC(C)CC[C@H](c1ccc(C(=O)O)cc1)N1C(=O)C(N2CCC(C(F)(F)F)CC2)=NC12CCC(C(C)(C)C)CC2] |
| 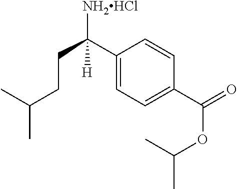 | 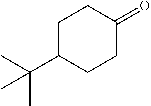 | 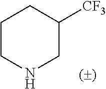 | A-57 | 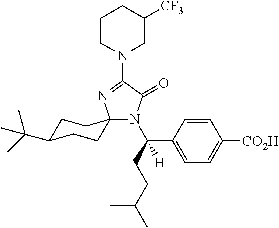 [SMILES: CC(C)CC[C@H](c1ccc(C(=O)O)cc1)N1C(=O)C(N2CCCC(C(F)(F)F)C2)=NC12CCC(C(C)(C)C)CC2] mixture of diastereomers |
| 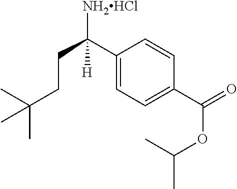 | 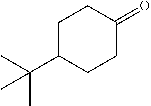 | 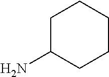 | A-58 | 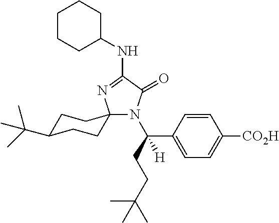 [SMILES: CC(C)(C)CC[C@H](c1ccc(C(=O)O)cc1)N1C(=O)C(NC2CCCCC2)=NC12CCC(C(C)(C)C)CC2] |
| 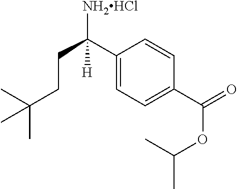 | 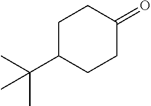 | 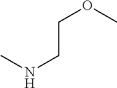 | A-59 | 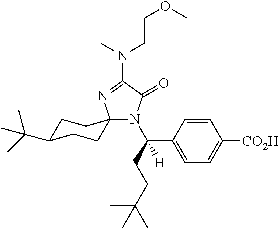 [SMILES: COCCN(C)C1=NC2(CCC(C(C)(C)C)CC2)N([C@H](CCC(C)(C)C)c2ccc(C(=O)O)cc2)C1=O] |
| 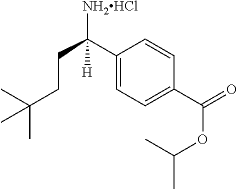 | 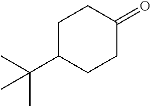 | 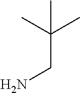 | A-60 | 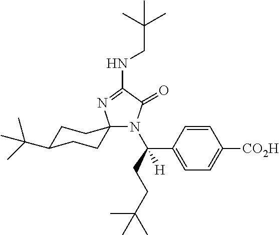 [SMILES: CC(C)(C)CC[C@H](c1ccc(C(=O)O)cc1)N1C(=O)C(NCC(C)(C)C)=NC12CCC(C(C)(C)C)CC2] |
| 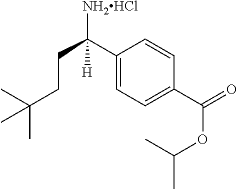 | 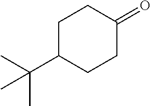 | 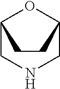 | A-61 | 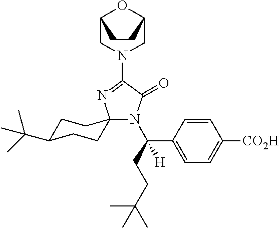 [SMILES: CC(C)(C)CC[C@H](c1ccc(C(=O)O)cc1)N1C(=O)C(N2C[C@H]3CC[C@@H](C2)O3)=NC12CCC(C(C)(C)C)CC2] |
| 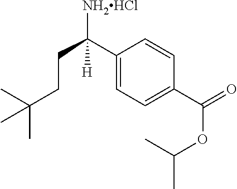 | 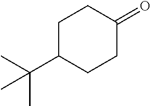 | 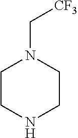 [SMILES: FC(F)(F)CN1CCNCC1] | A-62 | 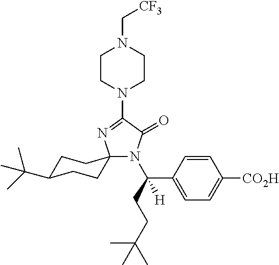 [SMILES: CC(C)(C)CC[C@H](c1ccc(C(=O)O)cc1)N1C(=O)C(N2CCN(CC(F)(F)F)CC2)=NC12CCC(C(C)(C)C)CC2] |
| 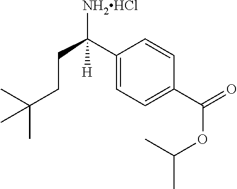 | 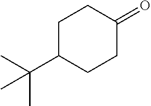 | 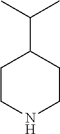 | A-63 | 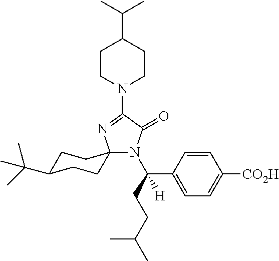 [SMILES: CC(C)CC[C@H](c1ccc(C(=O)O)cc1)N1C(=O)C(N2CCC(C(C)C)CC2)=NC12CCC(C(C)(C)C)CC2] |
| 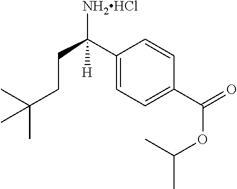 | 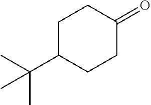 | 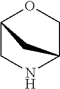 | A-64 | 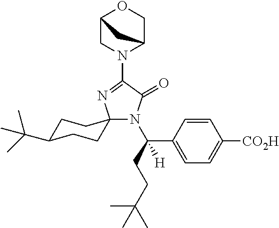 [SMILES: CC(C)(C)CC[C@H](c1ccc(C(=O)O)cc1)N1C(=O)C(N2C[C@H]3C[C@@H]2CO3)=NC12CCC(C(C)(C)C)CC2] |
| 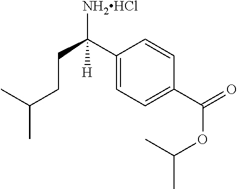 | 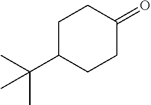 | 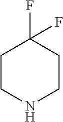 | A-65 | 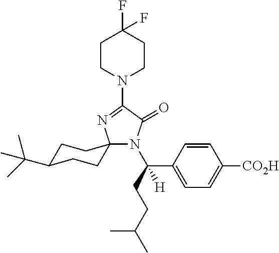 [SMILES: CC(C)CC[C@H](c1ccc(C(=O)O)cc1)N1C(=O)C(N2CCC(F)(F)CC2)=NC12CCC(C(C)(C)C)CC2] |
| 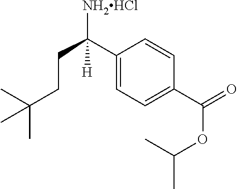 | 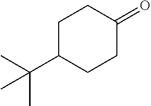 | 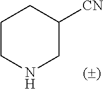 | A-66 | 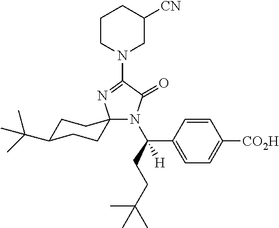 [SMILES: CC(C)(C)CC[C@H](c1ccc(C(=O)O)cc1)N1C(=O)C(N2CCCC(C#N)C2)=NC12CCC(C(C)(C)C)CC2] mixture of diastereomers |
| 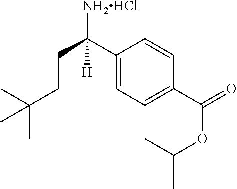 | 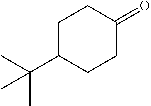 | 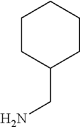 | A-67 | 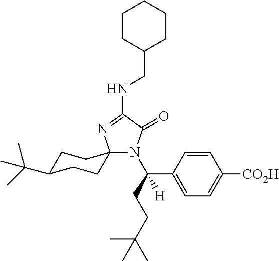 [SMILES: CC(C)(C)CC[C@H](c1ccc(C(=O)O)cc1)N1C(=O)C(NCC2CCCCC2)=NC12CCC(C(C)(C)C)CC2] |
| 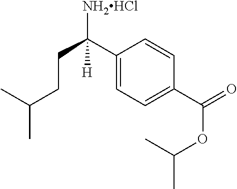 | 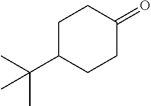 | 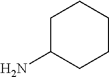 | A-68 | 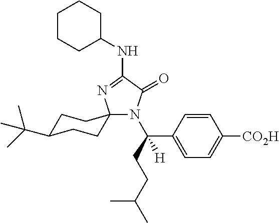 [SMILES: CC(C)CC[C@H](c1ccc(C(=O)O)cc1)N1C(=O)C(NC2CCCCC2)=NC12CCC(C(C)(C)C)CC2] |
| 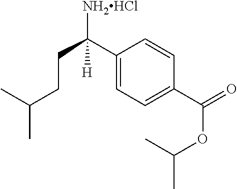 | 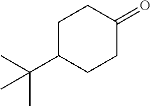 | 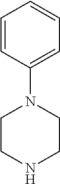 [SMILES: c1ccc(N2CCNCC2)cc1] | A-69 | 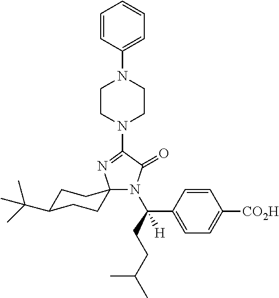 [SMILES: CC(C)CC[C@H](c1ccc(C(=O)O)cc1)N1C(=O)C(N2CCN(c3ccccc3)CC2)=NC12CCC(C(C)(C)C)CC2] |
| 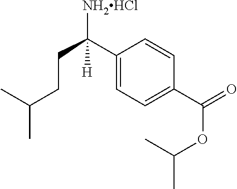 | 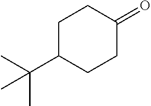 | 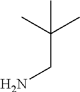 | A-70 | 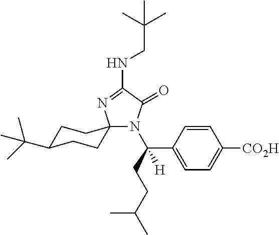 [SMILES: CC(C)CC[C@H](c1ccc(C(=O)O)cc1)N1C(=O)C(NCC(C)(C)C)=NC12CCC(C(C)(C)C)CC2] |
| 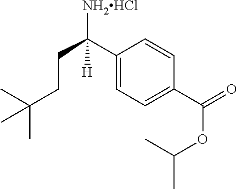 | 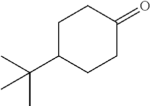 | 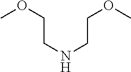 | A-71 | 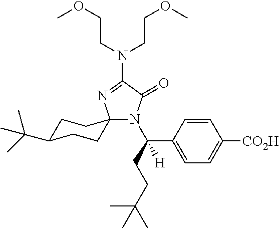 [SMILES: COCCN(CCOC)C1=NC2(CCC(C(C)(C)C)CC2)N([C@H](CCC(C)(C)C)c2ccc(C(=O)O)cc2)C1=O] |
| 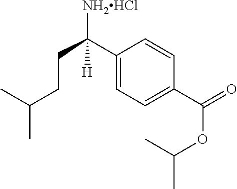 | 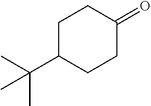 | 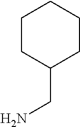 | A-72 | 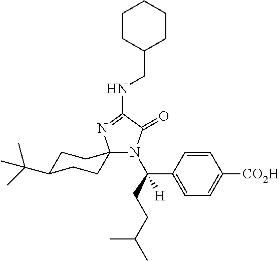 [SMILES: CC(C)CC[C@H](c1ccc(C(=O)O)cc1)N1C(=O)C(NCC2CCCCC2)=NC12CCC(C(C)(C)C)CC2] |
| 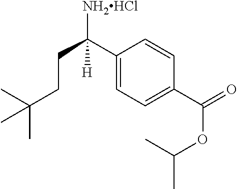 | 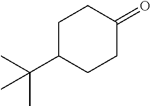 | 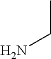 | A-73 | 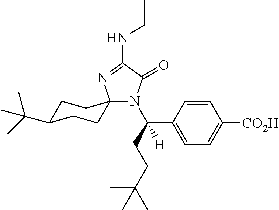 [SMILES: CCNC1=NC2(CCC(C(C)(C)C)CC2)N([C@H](CCC(C)(C)C)c2ccc(C(=O)O)cc2)C1=O] |
| 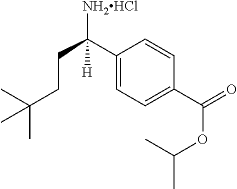 | 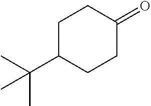 | 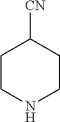 | A-74 | 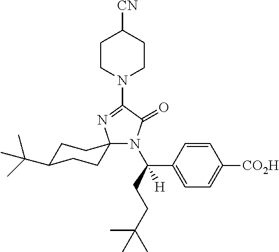 [SMILES: CC(C)(C)CC[C@H](c1ccc(C(=O)O)cc1)N1C(=O)C(N2CCC(C#N)CC2)=NC12CCC(C(C)(C)C)CC2] |
| 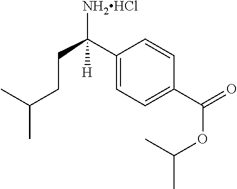 | 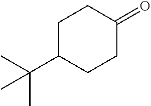 | 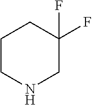 | A-75 | 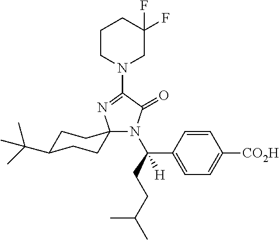 [SMILES: CC(C)CC[C@H](c1ccc(C(=O)O)cc1)N1C(=O)C(N2CCCC(F)(F)C2)=NC12CCC(C(C)(C)C)CC2] |
| 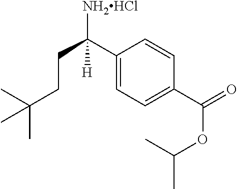 | 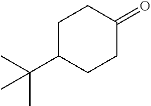 | 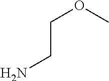 | A-76 | 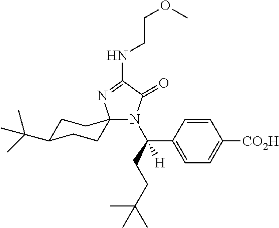 [SMILES: COCCNC1=NC2(CCC(C(C)(C)C)CC2)N([C@H](CCC(C)(C)C)c2ccc(C(=O)O)cc2)C1=O] |
| 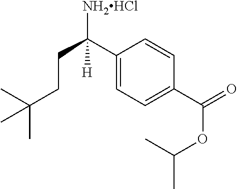 | 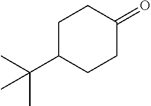 | 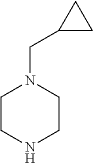 | A-77 | 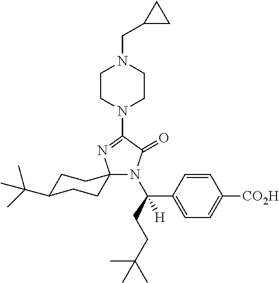 [SMILES: CC(C)(C)CC[C@H](c1ccc(C(=O)O)cc1)N1C(=O)C(N2CCN(CC3CC3)CC2)=NC12CCC(C(C)(C)C)CC2] |
| 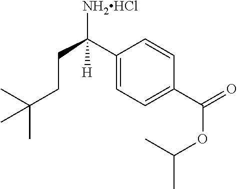 | 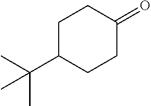 | 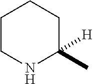 | A-78 | 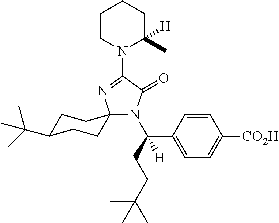 [SMILES: C[C@@H]1CCCCN1C1=NC2(CCC(C(C)(C)C)CC2)N([C@H](CCC(C)(C)C)c2ccc(C(=O)O)cc2)C1=O] |
| 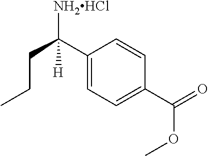 | 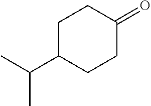 | 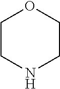 | A-79 | 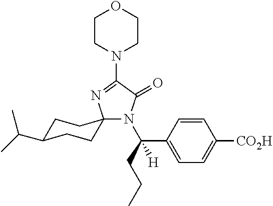 [SMILES: CCC[C@H](c1ccc(C(=O)O)cc1)N1C(=O)C(N2CCOCC2)=NC12CCC(C(C)C)CC2] |
| 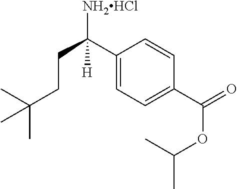 | 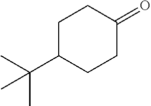 | 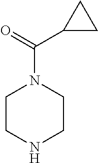 [SMILES: O=C(C1CC1)N1CCNCC1] | A-80 | 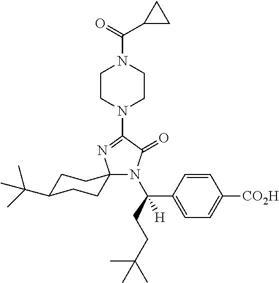 [SMILES: CC(C)(C)CC[C@H](c1ccc(C(=O)O)cc1)N1C(=O)C(N2CCN(C(=O)C3CC3)CC2)=NC12CCC(C(C)(C)C)CC2] |
| 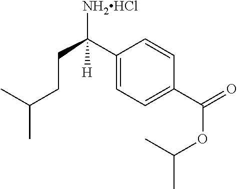 | 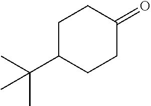 | 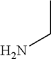 | A-81 | 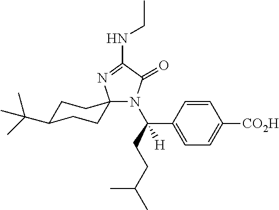 [SMILES: CCNC1=NC2(CCC(C(C)(C)C)CC2)N([C@H](CCC(C)C)c2ccc(C(=O)O)cc2)C1=O] |
| 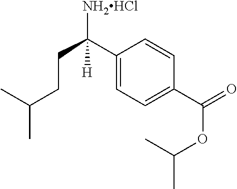 | 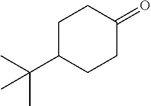 | 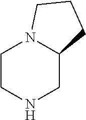 | A-82 | 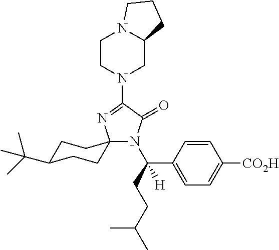 [SMILES: CC(C)CC[C@H](c1ccc(C(=O)O)cc1)N1C(=O)C(N2CCN3CCC[C@H]3C2)=NC12CCC(C(C)(C)C)CC2] |
| 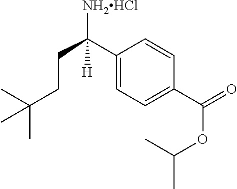 | 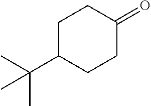 | 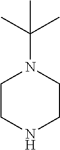 | A-83 | 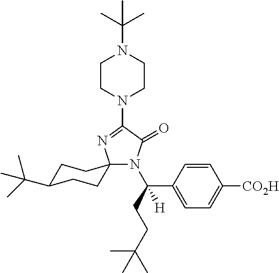 [SMILES: CC(C)(C)CC[C@H](c1ccc(C(=O)O)cc1)N1C(=O)C(N2CCN(C(C)(C)C)CC2)=NC12CCC(C(C)(C)C)CC2] |
| 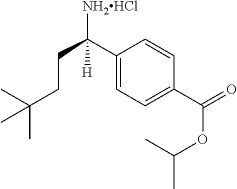 | 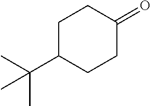 | 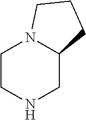 | A-84 | 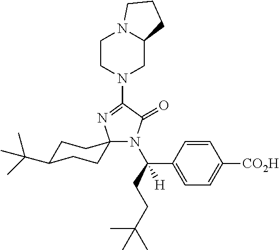 [SMILES: CC(C)(C)CC[C@H](c1ccc(C(=O)O)cc1)N1C(=O)C(N2CCN3CCC[C@H]3C2)=NC12CCC(C(C)(C)C)CC2] |
| 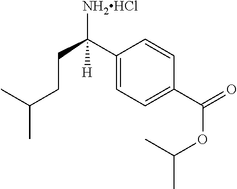 | 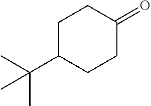 | 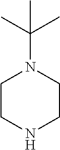 | A-85 | 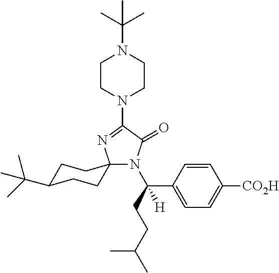 [SMILES: CC(C)CC[C@H](c1ccc(C(=O)O)cc1)N1C(=O)C(N2CCN(C(C)(C)C)CC2)=NC12CCC(C(C)(C)C)CC2] |
| 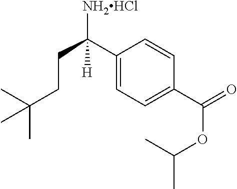 | 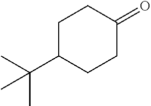 | 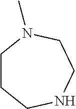 | A-86 | 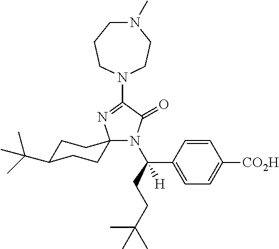 [SMILES: CN1CCCN(C2=NC3(CCC(C(C)(C)C)CC3)N([C@H](CCC(C)(C)C)c3ccc(C(=O)O)cc3)C2=O)CC1] |
| 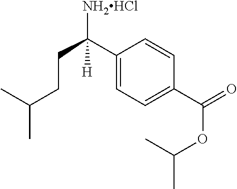 | 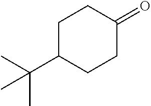 | 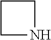 | A-87 | 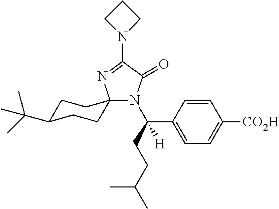 [SMILES: CC(C)CC[C@H](c1ccc(C(=O)O)cc1)N1C(=O)C(N2CCC2)=NC12CCC(C(C)(C)C)CC2] |
| 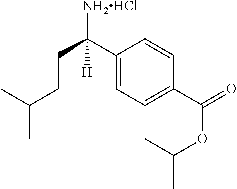 | 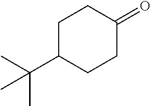 | 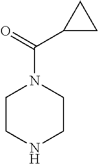 [SMILES: O=C(C1CC1)N1CCNCC1] | A-88 | 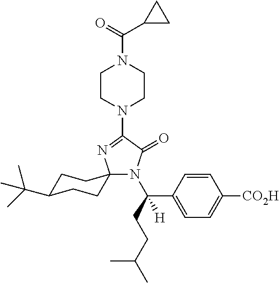 [SMILES: CC(C)CC[C@H](c1ccc(C(=O)O)cc1)N1C(=O)C(N2CCN(C(=O)C3CC3)CC2)=NC12CCC(C(C)(C)C)CC2] |
| 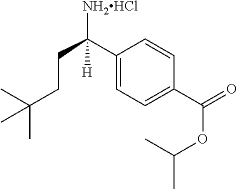 | 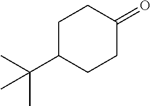 | 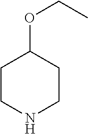 | A-89 | 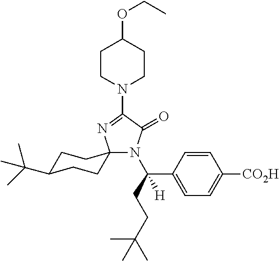 [SMILES: CCOC1CCN(C2=NC3(CCC(C(C)(C)C)CC3)N([C@H](CCC(C)(C)C)c3ccc(C(=O)O)cc3)C2=O)CC1] |
| 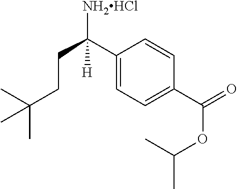 | 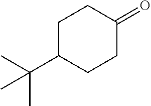 | 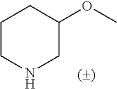 | A-90 | 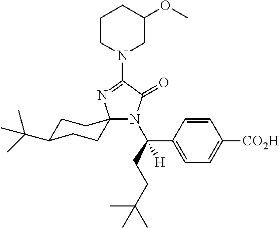 [SMILES: COC1CCCN(C2=NC3(CCC(C(C)(C)C)CC3)N([C@H](CCC(C)(C)C)c3ccc(C(=O)O)cc3)C2=O)C1] mixture of diastereomers |
| 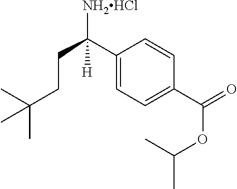 | 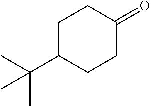 | 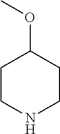 | A-91 | 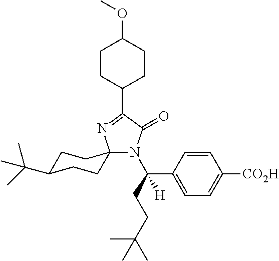 [SMILES: COC1CCC(C2=NC3(CCC(C(C)(C)C)CC3)N([C@H](CCC(C)(C)C)c3ccc(C(=O)O)cc3)C2=O)CC1] |
| 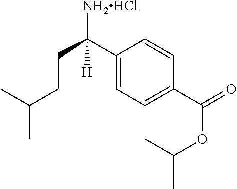 | 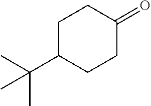 | 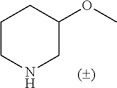 | A-92 | 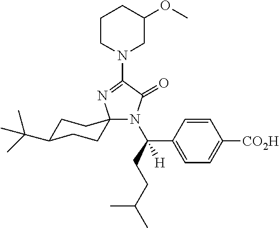 [SMILES: COC1CCCN(C2=NC3(CCC(C(C)(C)C)CC3)N([C@H](CCC(C)C)c3ccc(C(=O)O)cc3)C2=O)C1] mixture of diastereomers |
| 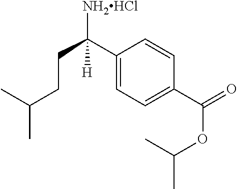 | 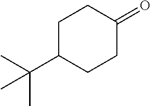 | 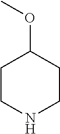 | A-93 | 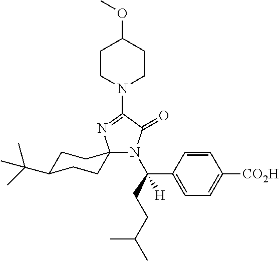 [SMILES: COC1CCN(C2=NC3(CCC(C(C)(C)C)CC3)N([C@H](CCC(C)C)c3ccc(C(=O)O)cc3)C2=O)CC1] |
| 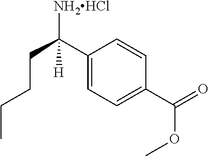 | 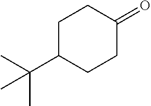 | 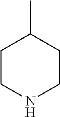 | A-94 | 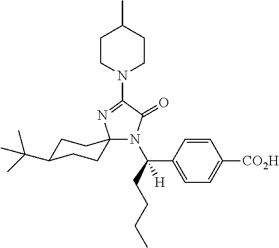 [SMILES: CCCC[C@H](c1ccc(C(=O)O)cc1)N1C(=O)C(N2CCC(C)CC2)=NC12CCC(C(C)(C)C)CC2] |
| 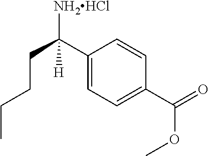 | 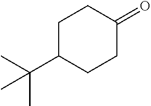 | 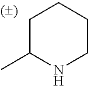 | A-95 | 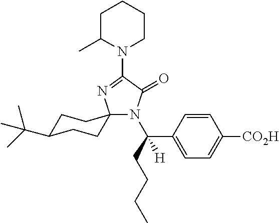 [SMILES: CCCC[C@H](c1ccc(C(=O)O)cc1)N1C(=O)C(N2CCCCC2C)=NC12CCC(C(C)(C)C)CC2] mixture of diastereomers |
| 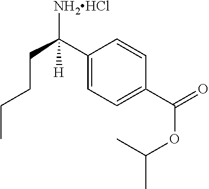 | 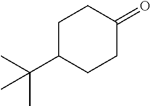 | 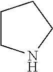 | A-96 | 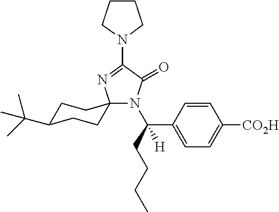 [SMILES: CCCC[C@H](c1ccc(C(=O)O)cc1)N1C(=O)C(N2CCCC2)=NC12CCC(C(C)(C)C)CC2] |
| 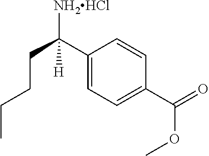 | 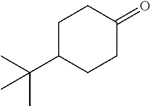 | 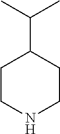 | A-97 | 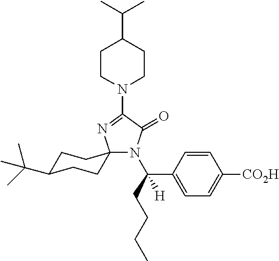 [SMILES: CCCC[C@H](c1ccc(C(=O)O)cc1)N1C(=O)C(N2CCC(C(C)C)CC2)=NC12CCC(C(C)(C)C)CC2] |
| 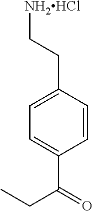 | 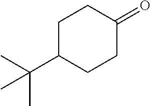 | — | A-98 | 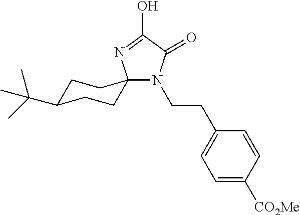 [SMILES: COC(=O)c1ccc(CCN2C(=O)C(O)=NC23CCC(C(C)(C)C)CC3)cc1] |

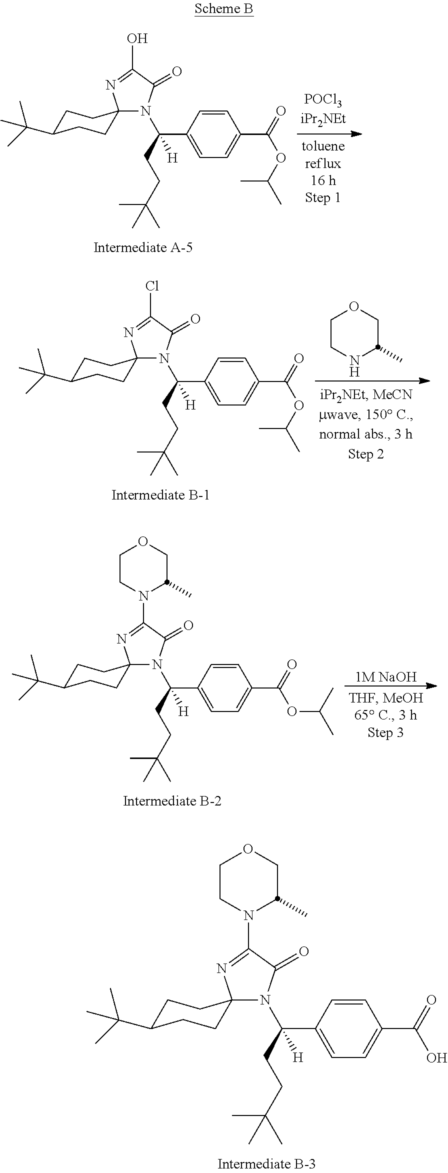

### Step 1

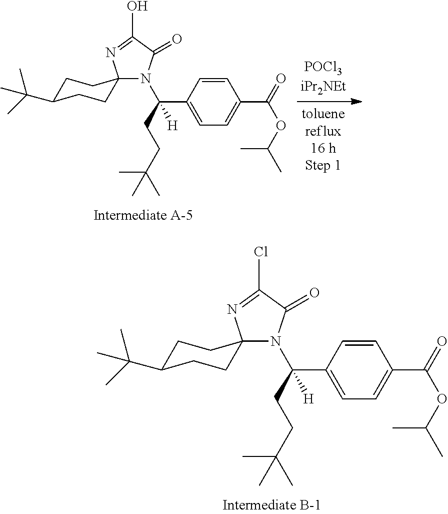

Phosphorus oxychloride (0.79 mL, 8.48 mmol) was added dropwise to a solution of Intermediate A-5 (1.37 g, 2.83 mmol) and N,N-diisopropylethylamine (2.95 mL, 17 mmol) in toluene (10 mL) at room temperature. The reaction was heated at reflux with stirring for 16 h. After cooling to room temperature, the reaction was diluted with CH₂Cl₂ and poured over ice. Brine was added to the quenched reaction, and the mixture was stirred for 10 minutes. The organic layer was removed and washed with brine. The aqueous layer was extracted with EtOAc. The EtOAc layer was washed with brine, combined with the CH₂Cl₂ layer, dried over anhydrous magnesium sulfate, filtered and evaporated to afford a crude residue. Silica gel chromatography (gradient elution, 0% to 100% EtOAc in hexanes) afforded Intermediate B-1 (1.3 g, 91%) as a tan foam.

### Step 2

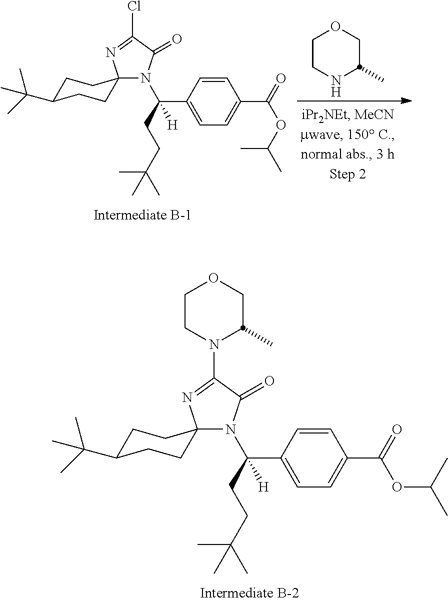

Intermediate B-1 (200 mg, 0.398 mmol, 1 eq), (S)-3-methylmorpholine (121 mg, 1.19 mmol, 3 eq), and iPr₂NEt (0.21 mL, 1.19 mmol, 3 eq) were dissolved in acetonitrile (2 mL) in a Biotage 0.5 mL-2 mL reaction vessel. The vessel was sealed and was subjected to microwave irradiation (normal absorption, 150° C., 3 h). After cooling the reaction to room temperature, the vessel was uncapped, and the reaction solution was subjected to reversed-phase C18 chromatography (gradient elution, 10% to 100% MeCN in H₂O with 0.1% HCOOH, Analogix 55 g C18 column, Biotage SP-1) to afford Intermediate B-2 (140 mg, 62%) as a film.

### Step 3

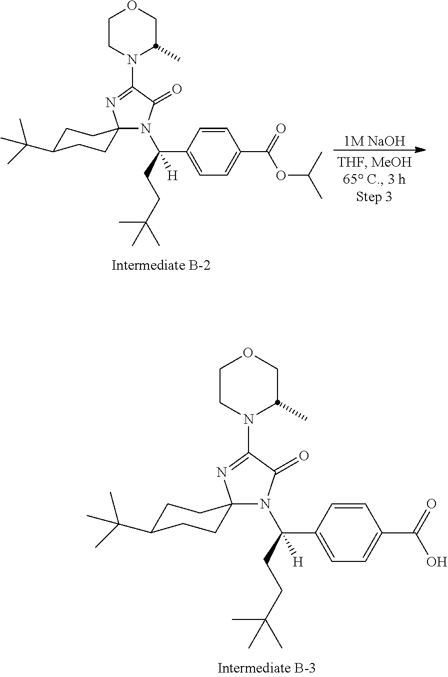

A solution of Intermediate B-2 (150 mg, 0.26 mmol) in THF (10 mL) and MeOH (10 mL) was treated with 1M NaOH (aq.) (5 mL). The reaction mixture was heated with stirring for 3 h at 65° C. After cooling to room temperature, the reaction mixture was partitioned between EtOAc and 1M HCl (aq.). The aqueous layer was discarded, and the organic layer was washed with brine, dried over anhydrous Na₂SO₄, filtered and evaporated to afford Intermediate B-3 (125 mg, 90% yield), which was used in the next step without further purification.

TABLE 2
Using the requisite starting materials, and a method similar to that outlined in
Scheme B, the following compounds were prepared:

| | | Inter- | |
|---|---|---|---|
| | | mediate | |
| Step 1 Starting Material | Amine | Number | Intermediate Prepared |
| 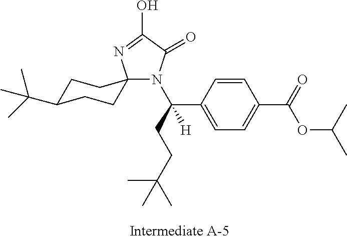 [SMILES: CC(C)OC(=O)c1ccc([C@@H](CCC(C)(C)C)N2C(=O)C(O)=NC23CCC(C(C)(C)C)CC3)cc1] | 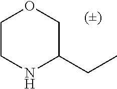 | B-4 | 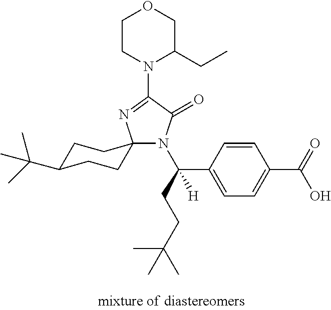 [SMILES: CCC1COCCN1C1=NC2(CCC(C(C)(C)C)CC2)N([C@H](CCC(C)(C)C)c2ccc(C(=O)O)cc2)C1=O] |
| 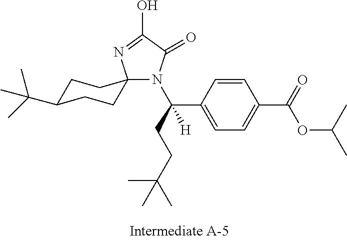 [SMILES: CC(C)OC(=O)c1ccc([C@@H](CCC(C)(C)C)N2C(=O)C(O)=NC23CCC(C(C)(C)C)CC3)cc1] | 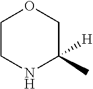 | B-5 | 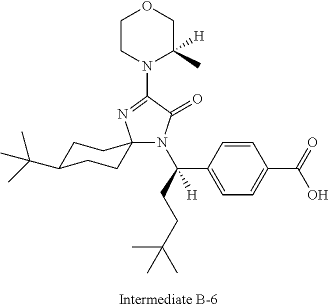 [SMILES: C[C@@H]1COCCN1C1=NC2(CCC(C(C)(C)C)CC2)N([C@H](CCC(C)(C)C)c2ccc(C(=O)O)cc2)C1=O] |
| 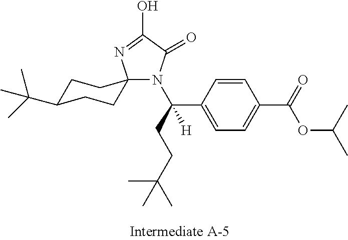 [SMILES: CC(C)OC(=O)c1ccc([C@@H](CCC(C)(C)C)N2C(=O)C(O)=NC23CCC(C(C)(C)C)CC3)cc1] | 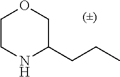 | B-6 | 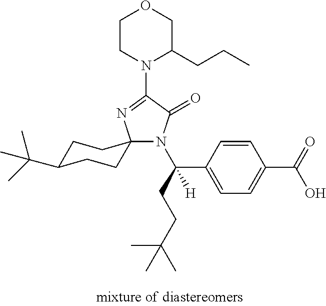 [SMILES: CCCC1COCCN1C1=NC2(CCC(C(C)(C)C)CC2)N([C@H](CCC(C)(C)C)c2ccc(C(=O)O)cc2)C1=O] |
| 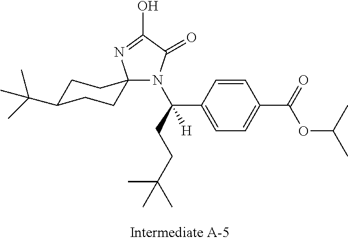 [SMILES: CC(C)OC(=O)c1ccc([C@@H](CCC(C)(C)C)N2C(=O)C(O)=NC23CCC(C(C)(C)C)CC3)cc1] | 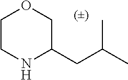 | B-7 | 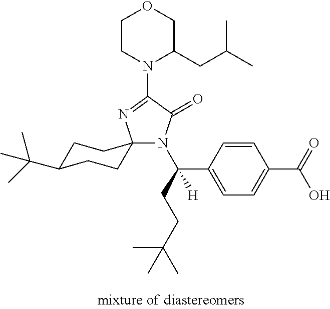 [SMILES: CC(C)CC1COCCN1C1=NC2(CCC(C(C)(C)C)CC2)N([C@H](CCC(C)(C)C)c2ccc(C(=O)O)cc2)C1=O] |
| 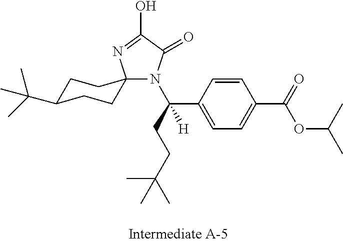 [SMILES: CC(C)OC(=O)c1ccc([C@@H](CCC(C)(C)C)N2C(=O)C(O)=NC23CCC(C(C)(C)C)CC3)cc1] | 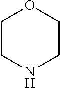 | B-8 | 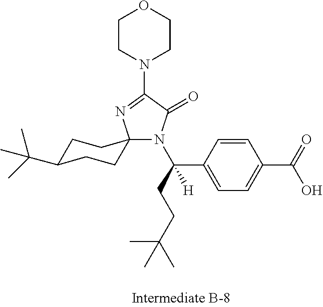 [SMILES: CC(C)(C)CC[C@H](c1ccc(C(=O)O)cc1)N1C(=O)C(N2CCOCC2)=NC12CCC(C(C)(C)C)CC2] |
| 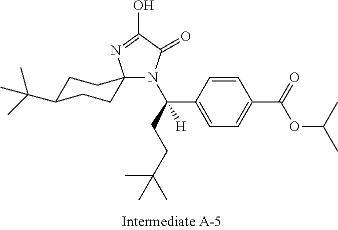 [SMILES: CC(C)OC(=O)c1ccc([C@@H](CCC(C)(C)C)N2C(=O)C(O)=NC23CCC(C(C)(C)C)CC3)cc1] | 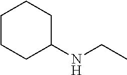 | B-9 | 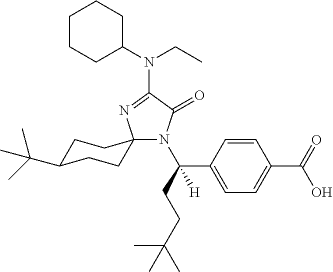 [SMILES: CCN(C1=NC2(CCC(C(C)(C)C)CC2)N([C@H](CCC(C)(C)C)c2ccc(C(=O)O)cc2)C1=O)C1CCCCC1] |
| 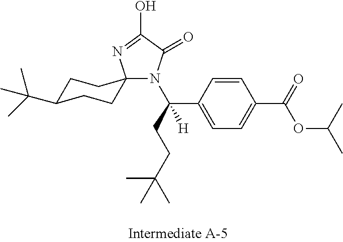 [SMILES: CC(C)OC(=O)c1ccc([C@@H](CCC(C)(C)C)N2C(=O)C(O)=NC23CCC(C(C)(C)C)CC3)cc1] | 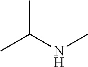 | B-10 | 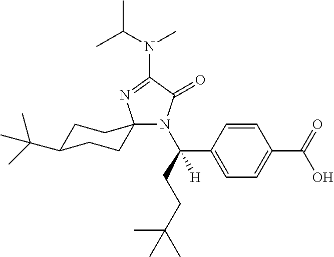 [SMILES: CC(C)N(C)C1=NC2(CCC(C(C)(C)C)CC2)N([C@H](CCC(C)(C)C)c2ccc(C(=O)O)cc2)C1=O] |
| 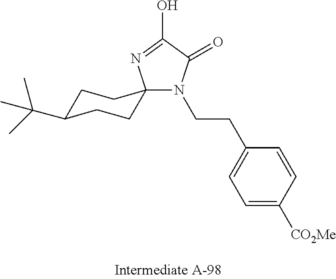 [SMILES: COC(=O)c1ccc(CCN2C(=O)C(O)=NC23CCC(C(C)(C)C)CC3)cc1] | 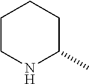 | B-11 | 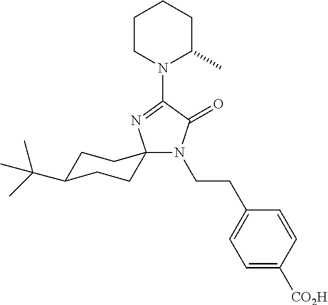 [SMILES: C[C@H]1CCCCN1C1=NC2(CCC(C(C)(C)C)CC2)N(CCc2ccc(C(=O)O)cc2)C1=O] |
| 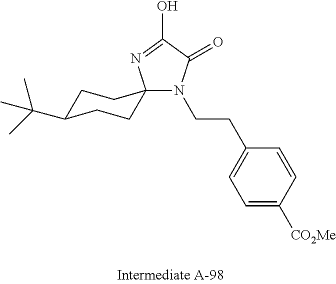 [SMILES: COC(=O)c1ccc(CCN2C(=O)C(O)=NC23CCC(C(C)(C)C)CC3)cc1] | 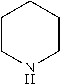 | B-12 | 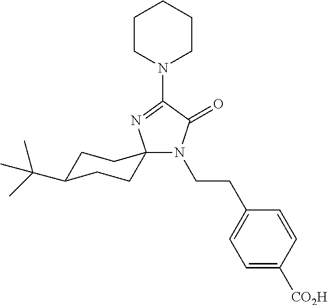 [SMILES: CC(C)(C)C1CCC2(CC1)N=C(N1CCCCC1)C(=O)N2CCc1ccc(C(=O)O)cc1] |

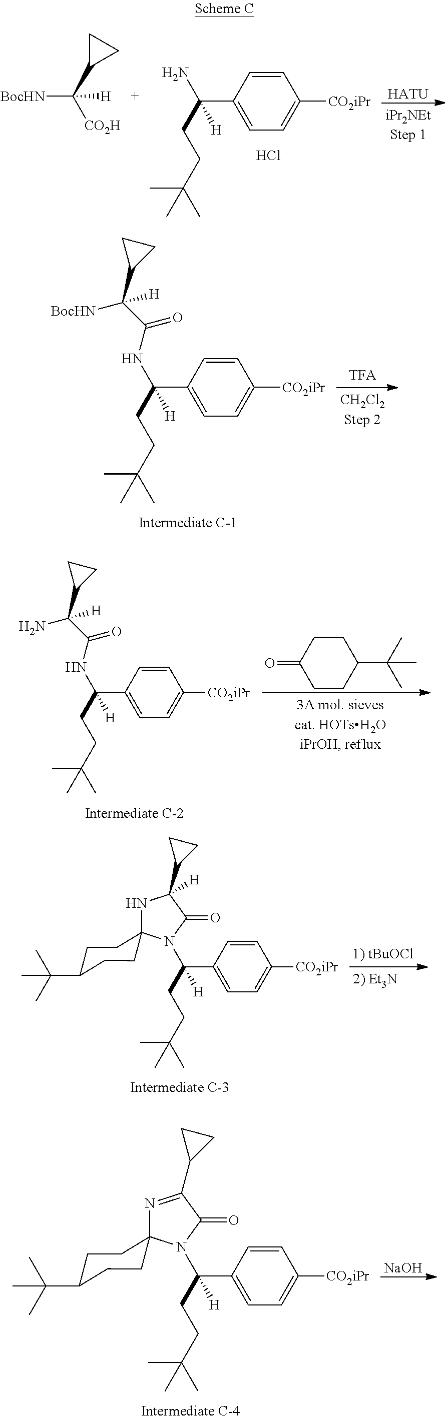

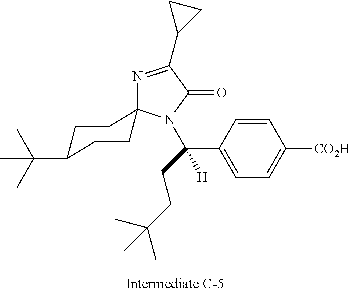 [SMILES: CC(C)(C)CC[C@H](c1ccc(C(=O)O)cc1)N1C(=O)C(C2CC2)=NC12CCC(C(C)(C)C)CC2]

### Step 1

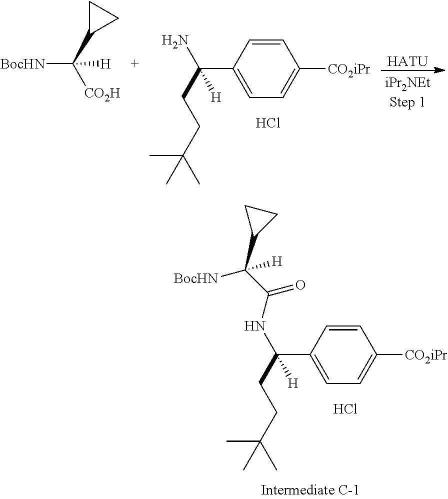

The amine (1.41 grams, 4.49 mmol, 1.00 eq), the N—BOC amino acid (0.966 g, 4.49 mmol, 1.00 eq), HATU (1.71 g, 4.49 mmol), and i-Pr₂NEt (2.3 mL, 13.5 mmol, 3 eq) were taken up in a mixture of CH₂Cl₂ (30 ml) and DMF (3 mL). The resulting solution was stirred at room temperature for 18 h. The reaction was concentrated, and the residue was partitioned between EtOAc and 1N HCl_{(aq.)}/brine. The aqueous layer was discarded, and the organic layer was washed with saturated NaHCO₃(aq.), then brine. The organic layer was dried over anhydrous sodium sulfate, filtered, and concentrated to afford a crude residue which was purified via silica gel chromagragphy (Analogix, gradient elution, 0-100% EtOAc in hexanes) to provide 1.77 g (83%) of Intermediate C-1.

### Step 2

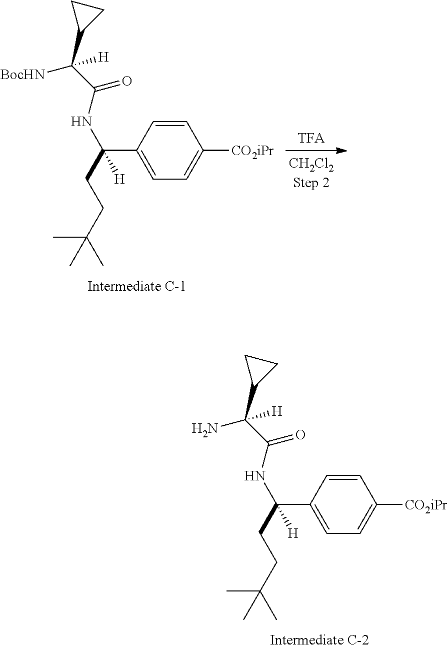

Intermediate C-1 (1.77 g, 3.73 mmol) was dissolved in CH₂Cl₂ (40 mL). Trifluoroacetic acid (10 ml) was added, and the solution was stirred at 25° C. for 3 h. The reaction was concentrated. The resulting residue was partitioned between CH₂Cl₂ and 1M NaOH_{(aq.)}. The organic layer was saved, and the aqueous layer was extracted with CH₂Cl₂. The combined organic layers were washed with brine, dried over anhydrous Na₂SO₄, filtered, and concentrated to afford intermediate C-2 (1.38 g, 99%) as a viscous oil, which was used in the subsequent step without further purification.

### Step 3

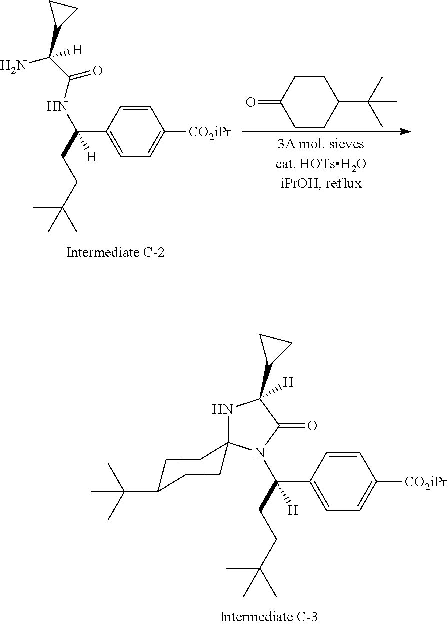

Intermediate C-2 (0.69 g, 1.84 mmol, 1 eq), 4-tert-butyl-cyclohexanone (0.284 g, 1.84 mmol, 1 eq), HOTs-H₂O (0.050 g, 0.26 mmol, 0.14 eq), and activated 3 Å mol. sieves (1.9 g, 8-12 mesh) were taken up in IPA (7 ml). The mixture was heated at reflux for 24 h. The reaction mixture was filtered and the filtrate was concentrated. The resulting residue was partitioned between EtOAc and saturated NaHCO₃(aq.). The aqueous layer was discarded and the organic layer was washed with brine, dried over anhydrous Na₂SO₄, filtered, and concentrated to afford Intermediate C-3 (0.88 g, 94%) as an off-white foam, which was used in the subsequent step without further purification.

### Step 4

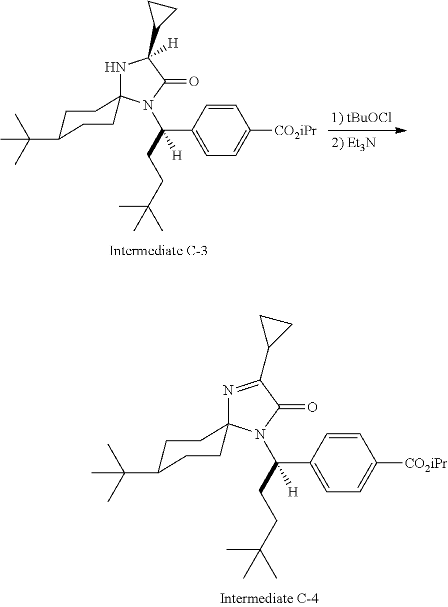

Intermediate C-3 (0.88 g, 1.7 mmol, 1.0 eq) was taken up in CH₂Cl₂ (20 ml), and t-BuOCl (0.243 mL, 2.14 mmol, 1.2 eq) was added dropwise at room temperature. After stirring for 75 minutes, Et₃N (1.0 mL, 7.14 mmol, 4.14 eq) was added, and the resulting solution was stirred at 25° C. for 1 h. The solution was diluted with CH₂Cl₂ and washed with 10% NaHSO_{3(aq)}. The aqueous layer was extracted with CH₂Cl₂. The combined organic layers were washed with brine, dried (anhydrous Na₂SO₄), filtered, and concentrated. The resulting residue was purified via gradient flash chromatography (Analogix, 0-20% EtOAc in hexanes, SiO₂) which provided an inseparable mixture of the desired product and chlorinated intermediate that had not undergone elimination. This mixture was dissolved in CH₂Cl₂ (10 mL) and was treated with iPr₂NEt (1.5 mL). The reaction was heated at reflux overnight. The reaction was partitioned between CH₂Cl₂ and 1M HCl_{(aq.)}. The organic layer was saved, and the aqueous layer was extracted with CH₂Cl₂. The combined organic layers were washed with brine, dried over anhydrous Na₂SO₄, filtered, and concentrated to afford a residue which was purified via gradient flash chromatography (Analogix, 0-40% EtOAc in hexanes, SiO₂) to provide Intermediate C-4 (0.52 g, 59%).

### Step 5

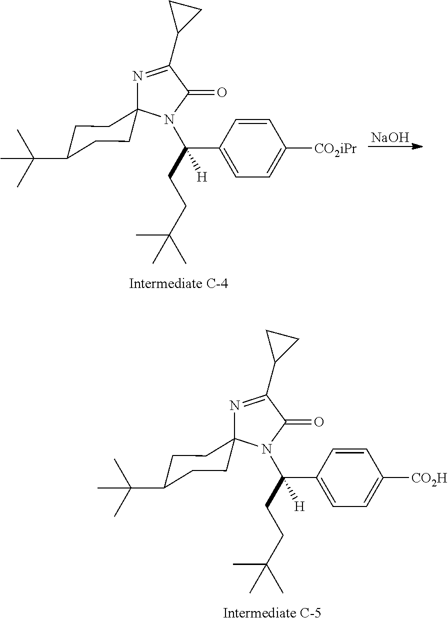

Intermediate C-4 (0.52 g, 1.01 mmol) was taken up in THF/MeOH/1 N NaOH_{(aq.)} (10/5/5 mL), and the resulting solution was stirred at 25° C. for 18 h. The reaction was partitioned between CH₂Cl₂ and 1 M HCl_{(aq.)}. The organic layer was saved, and the aqueous layer was extracted with CH₂Cl₂. The combined organic layers were washed with brine, dried over anhydrous Na₂SO₄, filtered, and concentrated to afford Intermediate C-5 (0.44 g, 93%) which was used in the next step without further purification.

TABLE 3
Using the requisite starting materials, and a method similar to that outlined in
Scheme C, the following compounds were prepared:

| | | | Inter- | |
|---|---|---|---|---|
| | | Boc- | medi- | |
| | | Protected | ate | |
| | | Amino | Num- | |
| Amine | Ketone | Acid | ber | Intermediate Prepared |
| 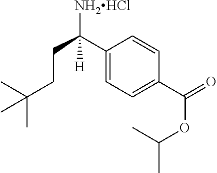 | 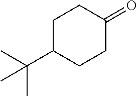 | 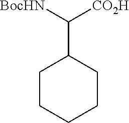 [SMILES: CC(C)(C)OC(=O)NC(C(=O)O)C1CCCCC1] | C-6 | 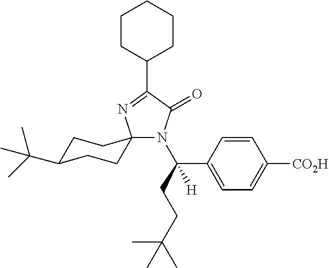 [SMILES: CC(C)(C)CC[C@H](c1ccc(C(=O)O)cc1)N1C(=O)C(C2CCCCC2)=NC12CCC(C(C)(C)C)CC2] |

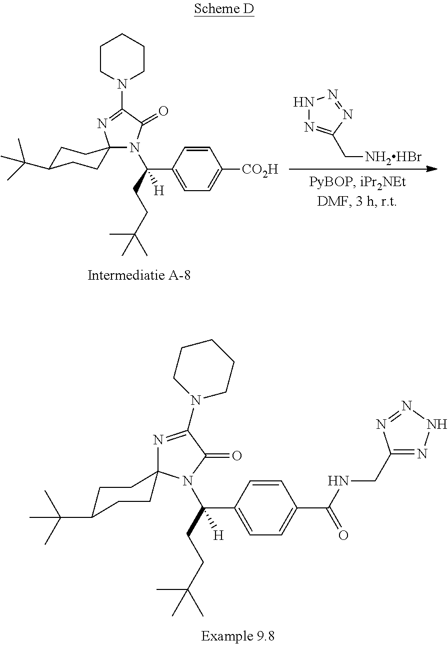

Intermediate A-8 (34 mg, 0.067 mmol, 1 eq), (2H-tetrazol-5-yl)methanamine hydrobromide (18 mg, 0.10 mmol, 1.5 eq), iPr₂NEt (0.035 mL, 0.20 mmol, 3 eq), and PyBOP (42 mg, 0.080 mmol, 1.2 eq) were combined in DMF (1 mL) and were stirred at room temperature for 3 hours. The solvent was removed in vacuo to afford a crude residue which was dissolved in DMSO and purified via reversed-phase C18 chromatography (Biotage SP-1, 55 g Analogix C18 column, gradient elution, 10% MeCN in water with 0.1% HCOOH to 100% MeCN with 0.1% HCOOH) to afford Example 9.8 (30 mg, 70%).

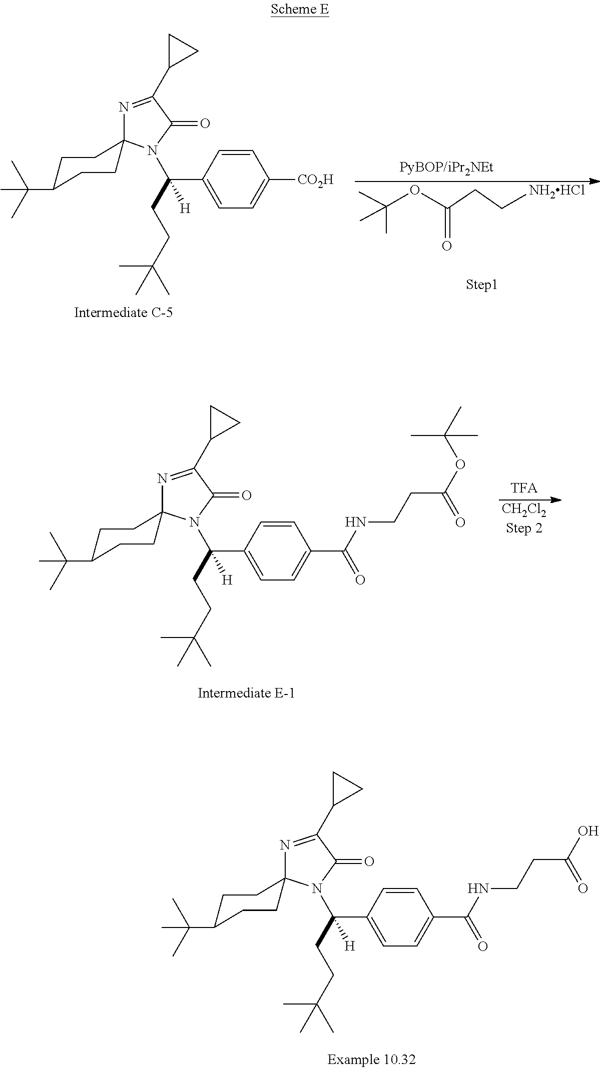

### Step 1

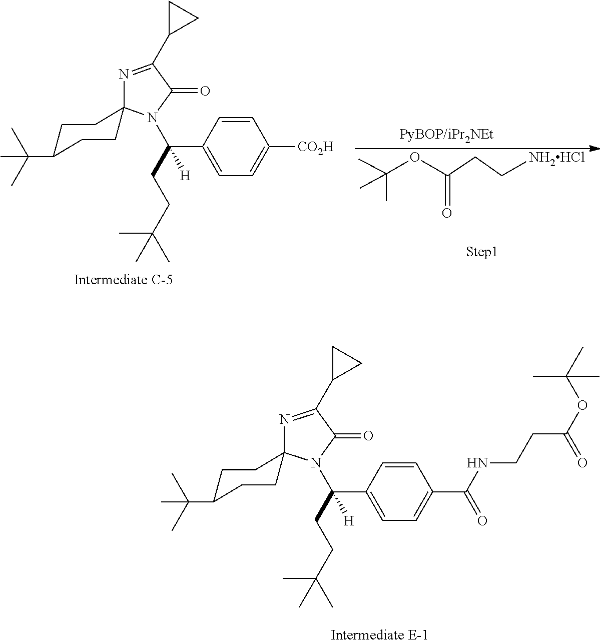

A mixture of Intermediate C-5 (0.13 g, 0.27 mmol, 1 eq), PyBOP (0.14 g, 0.27 mmol, 1 eq), tert-butyl 3-aminopropanoate hydrochloride (0.50 g, 0.27 mmol, 1 eq) and iPr₂NEt (0.14 mL, 0.82 mmol, 3.0 eq) in DMF (5 mL) and CH₂Cl₂ (2 mL) was stirred overnight at room temperature. The reaction mixture was partitioned between EtOAc and 1N HCl(aq.)/brine. The aqueous layer was discarded and the organic layer was washed with saturated NaHCO₃(aq.) and brine. The organic layer was dried over anhydrous Na₂SO₄, filtered, and evaporated to afford a crude residue which was purified via silica gel chromatography (gradient elution, 0% to 100% EtOAc in hexanes, Analogix) to provide Intermediate E-1 (146 mg, 90%).

### Step 2

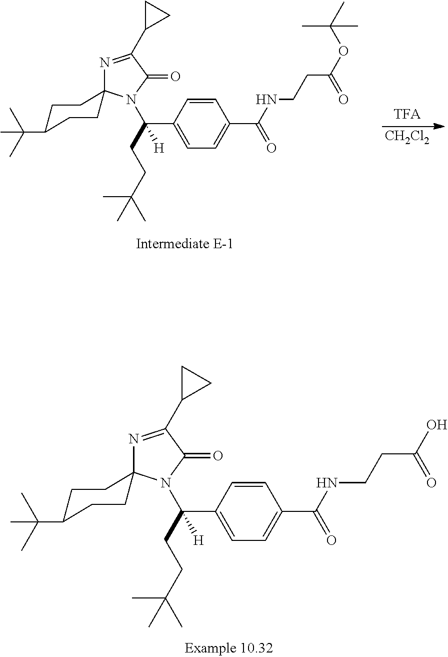

Intermediate E-1 (146 mg, 0.25 mmol) was dissolved in CH₂Cl₂ (7 mL), Trifluoroacetic acid (3 mL) was added and the reaction was stirred at room temperature for 3 h. The reaction mixture was concentrated to afford a crude residue which was purified via reversed-phase C18 column chromatography (Analogix 55 g C18 column, Biotage SP1 chromatography system, gradient elution 10% to 100% MeCN in H₂O with 0.1% HCOOH) to afford Example 10.32 (130 mg, 98%) as a white foam.

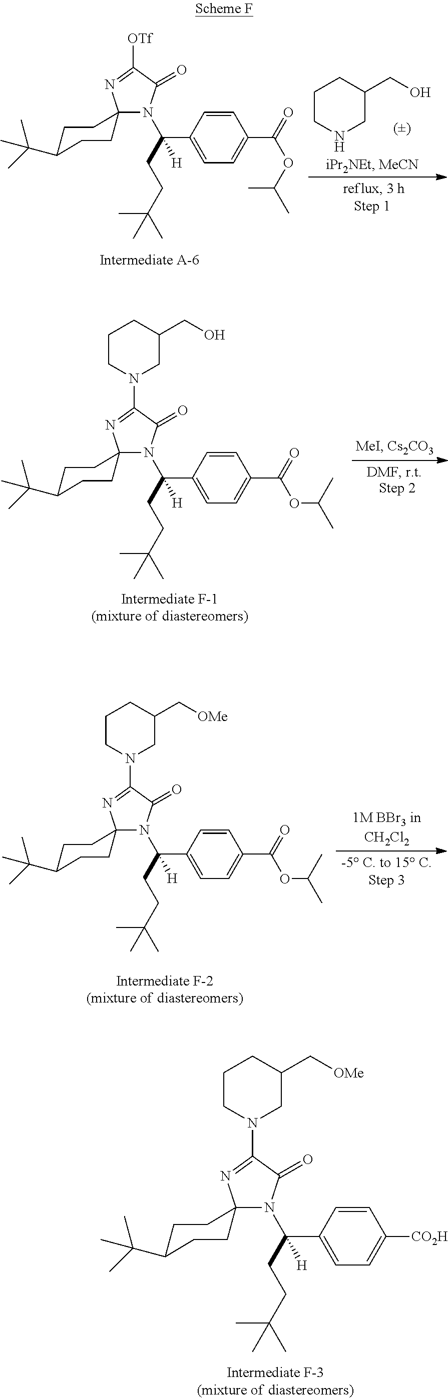

### Step 1

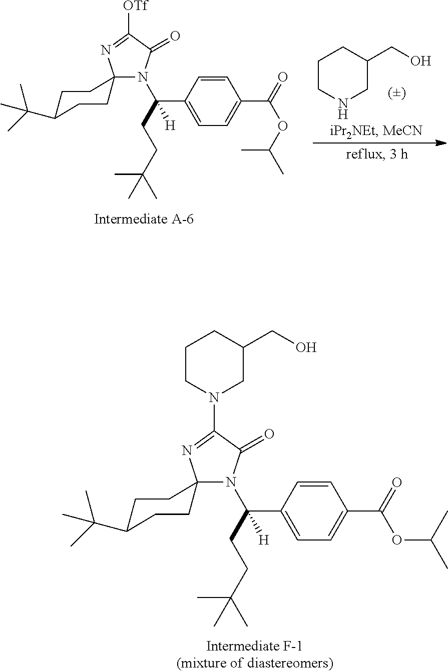

A solution of Intermediate A-6 (340 mg, 0.55 mmol, 1 eq), piperidin-3-ylmethanol (253 mg, 2.20 mmol, 4 eq), and iPr₂NEt (0.31 mL, 1.65 mmol, 3 eq) in MeCN (8 mL) was heated at reflux for 2 h. The reaction was concentrated and the resulting residue purified via silica gel chromatography (gradient elution, 20% to 100% EtOAc in hexanes) to afford Intermediate F-1 (297 mg, 92%, mixture of diastereomers) as a white foam.

### Step 2

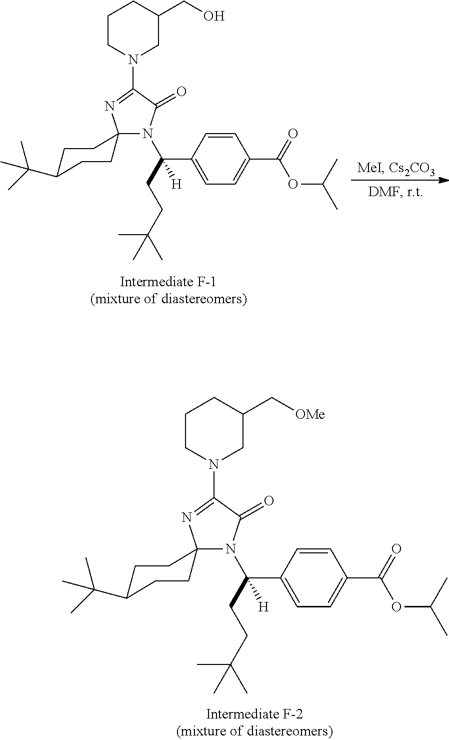

A solution of Intermediate F-1 (100 mg, 0.17 mmol, 1 eq), methyl iodide (73 mg, 0.52 mmol, 3 eq), and cesium carbonate (112 mg, 0.34 mmol, 2 eq) in DMF (3 mL) was stirred overnight at room temperature. The reaction was partitioned between EtOAc and brine. The aqueous layer was discarded and the organic layer was washed twice with brine, dried over anhydrous sodium sulfate, filtered, and evaporated to afford a crude residue. This crude material was treated with methyl iodide (241 mg, 1.7 mmol, 10 eq), and cesium carbonate (112 mg, 0.34 mmol, 2 eq) in DMSO (3 mL) and was stirred for 48 h at room temperature. The reaction was partitioned between EtOAc and brine. The aqueous layer was discarded and the organic layer was washed twice with brine, dried over anhydrous sodium sulfate, filtered, and evaporated to afford a crude residue. Silica gel chromatography (gradient elution, 0% to 30% EtOAc in hexanes) afforded Intermediate F-2 (72 mg, 70%, mixture of diastereomers) as a colorless thick oil.

### Step 3

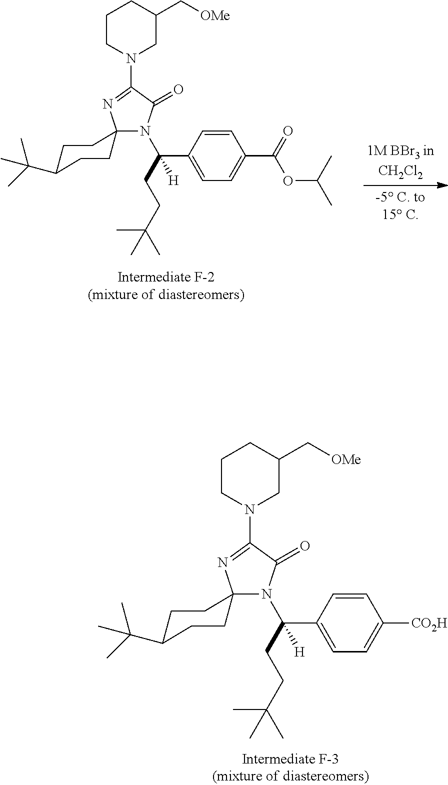

A solution of Intermediate F-2 (90 mg, 0.15 mmol, 1 eq) in CH₂Cl₂ (5 mL) at 0° C. was treated with 1M BBr₃ in CH₂Cl₂ (0.76 mL, 0.76 mmol, 5 eq). The reaction was stirred at 0° C. for 2 h and was then stirred at 10° C. for 2 h. The reaction was quenched with water. After partitioning between EtOAc and brine, the aqueous layer was removed. The organic layer was dried over anhydrous Na₂SO₄, filtered, and evaporated to afford intermediate F-3 (93 mg, quant., mixture of diastereomers) as a thick oil which was used in the next step without further purification.

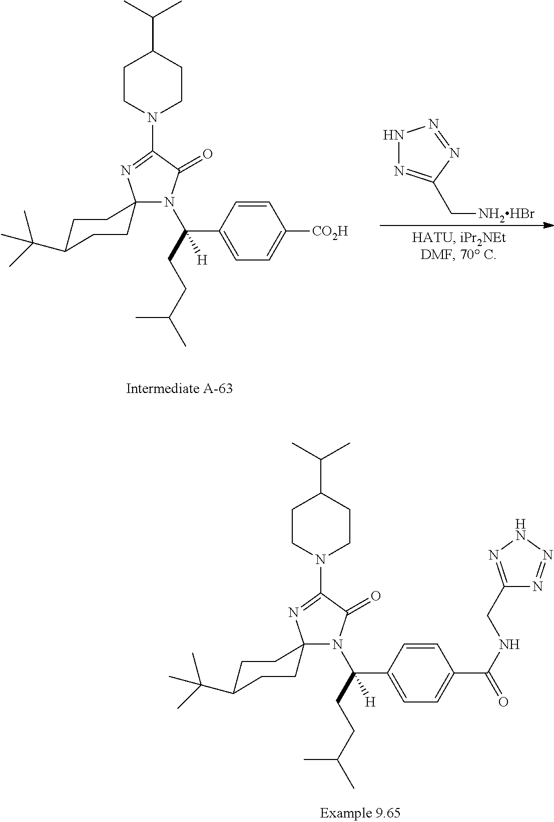

A solution of Intermediate A-63 (43 mg, 0.081 mmol, 1 eq), HATU (64 mg, 0.16 mmol, 2 eq), iPr₂NEt (0.054 mL, 0.32 mmol, 4 eq), and (2H-tetrazol-5-yl)methanamine hydrobromide (29 mg, 0.16 mmol, 2 eq) in DMF (3 mL) was stirred 5 h at 40° C. The crude reaction mixture was purified via reversed-phase C18 chromatography (gradient elution, 10% MeCN in water with 0.05% TFA to 100% MeCN with 0.05% TFA) to afford Example 9.65 (24 mg, 48%).

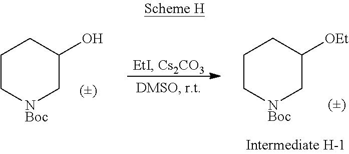 [SMILES: CC(C)(C)OC(=O)N1CCCC(O)C1 ; CCOC1CCCN(C(=O)OC(C)(C)C)C1]

A solution of tert-butyl 3-hydroxypiperidine-1-carboxylate (500 mg, 2.48 mmol, 1 eq), ethyl iodide (1.16 g, 7.44 mmol, 3 eq), and cesium carbonate (1.62 g, 4.96 mmol, 2 eq) were combined in DMSO (8 mL) and stirred for 2 days at room temperature. The reaction was partitioned between EtOAc and brine. The aqueous layer was discarded and the organic layer was washed three times with brine and evaporated to afford a crude residue. Silica gel chromatography (gradient elution, 0% to 20% EtOAc in hexanes) afforded Intermediate H-1 (210 mg, 37%) as a colorless, viscous oil.

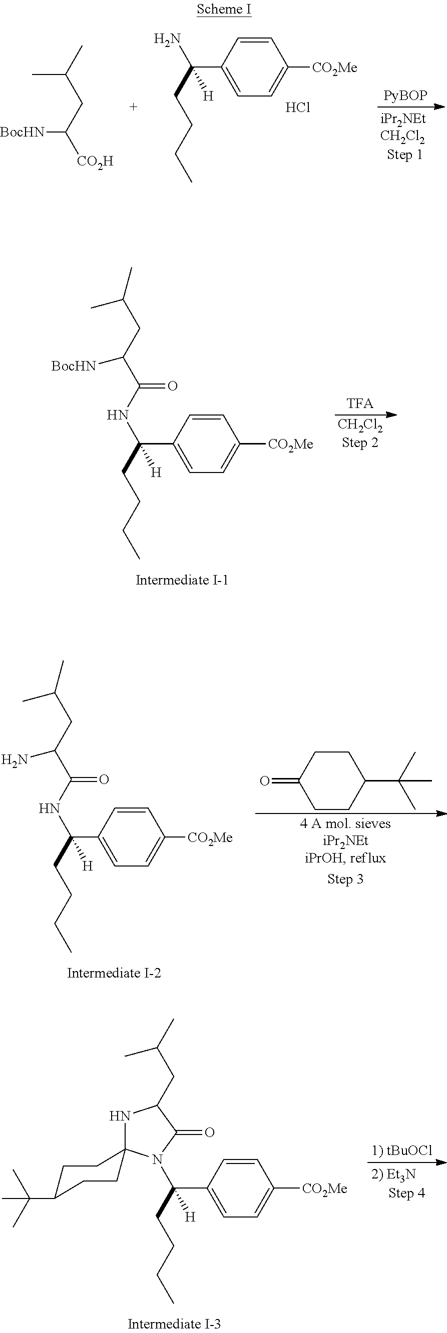

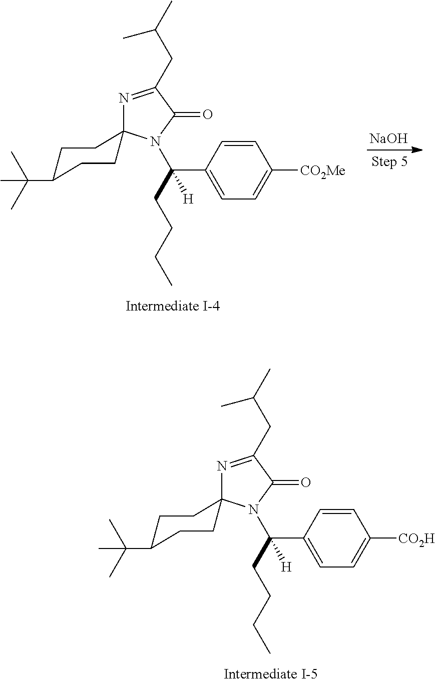 [SMILES: CCCC[C@H](c1ccc(C(=O)OC)cc1)N1C(=O)C(CC(C)C)=NC12CCC(C(C)(C)C)CC2 ; "NaOH" ; CCCC[C@H](c1ccc(C(=O)O)cc1)N1C(=O)C(CC(C)C)=NC12CCC(C(C)(C)C)CC2]

### Step 1

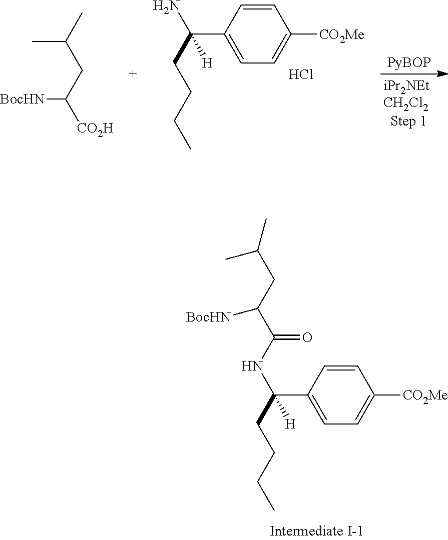 [SMILES: CC(C)CC(NC(=O)OC(C)(C)C)C(=O)O ; CCCC[C@@H](N)c1ccc(C(=O)OC)cc1 ; Cl ; CCCC[C@@H](NC(=O)C(CC(C)C)NC(=O)OC(C)(C)C)c1ccc(C(=O)OC)cc1]

The amine (300 mg, 1.23 mmol, 1.0 eq), the N—BOC amino acid (342 mg, 1.48 mmol, 1.2 eq), PyBOP (767 mg, 1.48 mmol, 1.2 eq), and i-Pr₂NEt (0.66 mL, 3.69 mmol, 3 eq) were taken up in CH₂Cl₂ (25 ml). The resulting solution was stirred at room temperature for 18 h. The reaction was concentrated, and the residue was partitioned between EtOAc and 1N NaOH_{(aq.)}. The aqueous layer was discarded, and the organic layer was dried over anhydrous sodium sulfate, filtered, and concentrated to afford a crude residue which was purified via silica gel chromagragphy (ISCO, gradient elution, 0-70% EtOAc in hexanes) to provide 550 mg (99%) of Intermediate I-1.

### Step 2

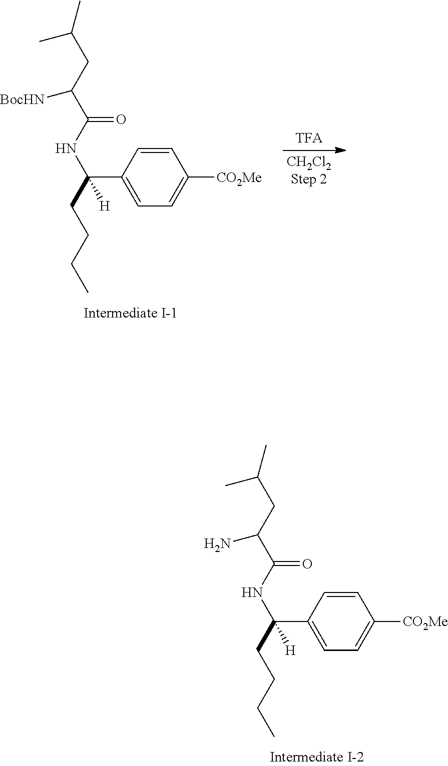 [SMILES: CCCC[C@@H](NC(=O)C(CC(C)C)NC(=O)OC(C)(C)C)c1ccc(C(=O)OC)cc1 ; "CH2Cl2" ; CCCC[C@@H](NC(=O)C(N)CC(C)C)c1ccc(C(=O)OC)cc1]

Intermediate I-1 (550 mg, 1.3 mmol) was dissolved in CH₂Cl₂ (20 mL). Trifluoroacetic acid (2 ml) was added, and the solution was stirred at 25° C. for 4 h. The reaction was concentrated. The resulting residue was partitioned between EtOAc and 1M NaOH_{(aq.)}. The organic layer was saved, and the aqueous layer was extracted with EtOAc. The combined organic layers were dried over anhydrous Na₂SO₄, filtered, and concentrated to afford intermediate I-2 (390 mg, 66%), which was used in the subsequent step without further purification.

### Step 3

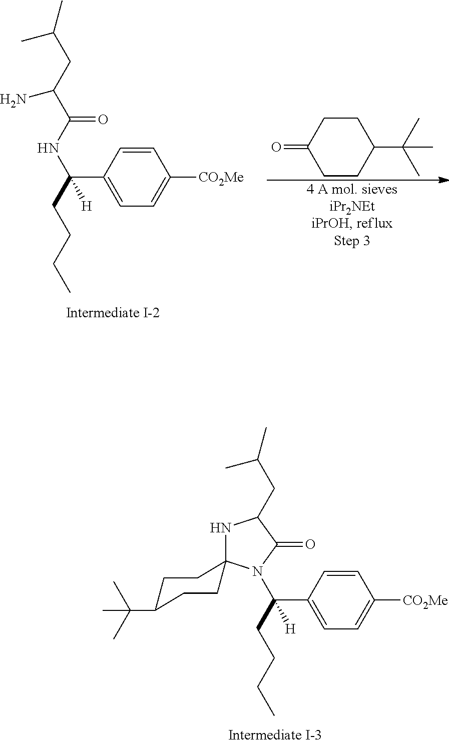

Intermediate I-2 (390 mg, 1.16 mmol, 1 eq), 4-tert-butyl-cyclohexanone (360 mg, 2.32 mmol, 2 eq), iPr₂NEt (1.24 mL, 6.96 mmol, 6 eq), and activated 4A mol. sieves (1 g, powdered) were taken up in isopropanol (30 ml). The mixture was heated at reflux for 18 h. The reaction mixture was filtered and the filtrate was concentrated. The resulting residue was purified via silica gel chromagragphy (ISCO, 40 g column, gradient elution, 0-50% EtOAc in hexanes) to afford Intermediate I-3 (400 mg, 73%).

### Step 4

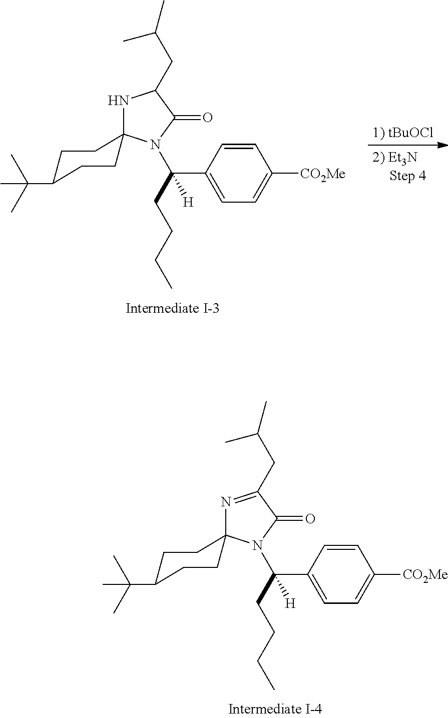 [SMILES: CCCC[C@H](c1ccc(C(=O)OC)cc1)N1C(=O)C(CC(C)C)NC12CCC(C(C)(C)C)CC2 ; CCCC[C@H](c1ccc(C(=O)OC)cc1)N1C(=O)C(CC(C)C)=NC12CCC(C(C)(C)C)CC2]

Intermediate I-3 (400 mg, 0.85 mmol, 1.0 eq) was taken up in CH₂Cl₂ (20 ml), and t-BuOCl (184 mg, 1.70 mmol, 2 eq) was added dropwise at room temperature. After stirring for 90 minutes, the reaction was cooled to 0° C. and Et₃N (0.34 mL, 2.55 mmol, 3 eq) was added. The resulting solution was warmed to 25° C. and stirred for 1 h. The solution was quenched with 10% NaHSO_{3(aq.)}. The aqueous layer was extracted with EtOAc. The combined organic layers were dried (anhydrous Na₂SO₄), filtered, and concentrated. The resulting residue was purified via gradient flash chromatography (ISCO, 40 g column, 0-30% EtOAc in hexanes, SiO₂) to provide Intermediate I-4 (129 mg).

### Step 5

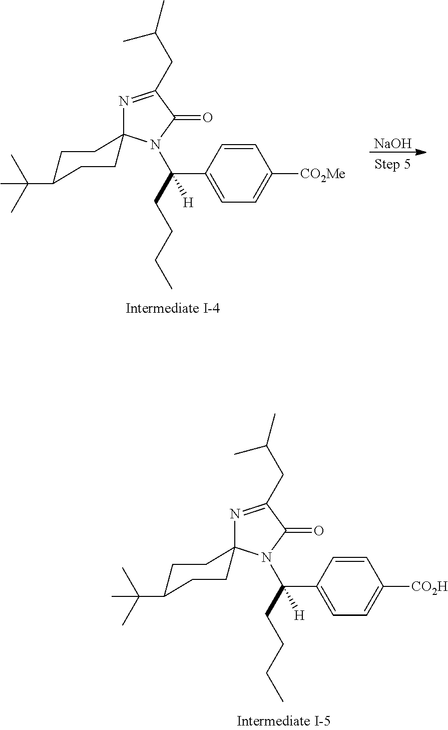 [SMILES: CCCC[C@H](c1ccc(C(=O)OC)cc1)N1C(=O)C(CC(C)C)=NC12CCC(C(C)(C)C)CC2 ; "NaOH" ; CCCC[C@H](c1ccc(C(=O)O)cc1)N1C(=O)C(CC(C)C)=NC12CCC(C(C)(C)C)CC2]

Intermediate I-4 (129 mg, 0.28 mmol) was taken up in THF:MeOH:2N NaOH_{(aq.)} (8:2:2 mL), and the resulting solution was stirred at 25° C. for 4 h. The reaction was concentrated to ˜⅓ the volume and was adjusted to ˜pH 3 with 1 M HCl_{(aq.)}. The aqueous layer was extracted with EtOAc. The combined organic layers were dried over anhydrous Na₂SO₄, filtered, and concentrated to afford Intermediate I-5 (109 mg, 89%) which was used in the next step without further purification.

TABLE 6
Using the requisite starting materials, and a method similar to that outlined in
Scheme I, the following compounds were prepared:

| Amine | Ketone | Boc-Protected Amino Acid | Intermediate Number | Intermediate Prepared |
|---|---|---|---|---|
| 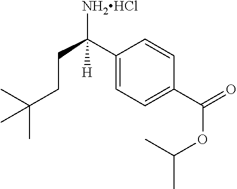 | 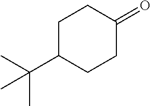 | 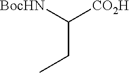 | I-6 | 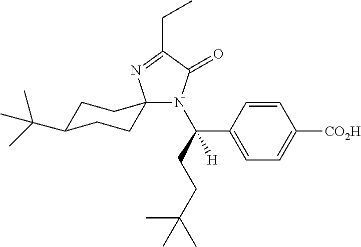 [SMILES: CCC1=NC2(CCC(C(C)(C)C)CC2)N([C@H](CCC(C)(C)C)c2ccc(C(=O)O)cc2)C1=O] |
| 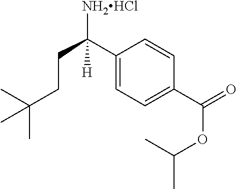 | 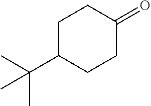 | 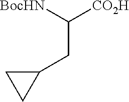 [SMILES: CC(C)(C)OC(=O)NC(CC1CC1)C(=O)O] | I-7 | 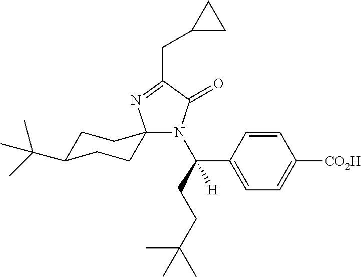 [SMILES: CC(C)(C)CC[C@H](c1ccc(C(=O)O)cc1)N1C(=O)C(CC2CC2)=NC12CCC(C(C)(C)C)CC2] |
| 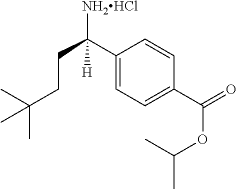 | 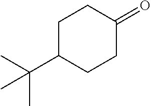 | 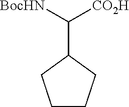 [SMILES: CC(C)(C)OC(=O)NC(C(=O)O)C1CCCC1] | I-8 | 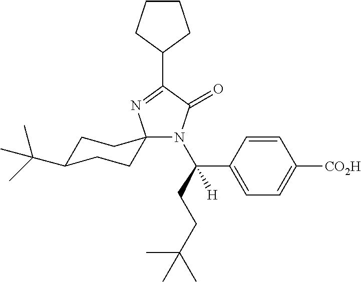 [SMILES: CC(C)(C)CC[C@H](c1ccc(C(=O)O)cc1)N1C(=O)C(C2CCCC2)=NC12CCC(C(C)(C)C)CC2] |
| 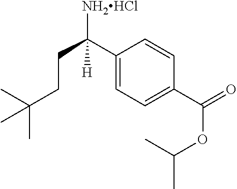 | 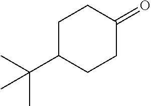 | 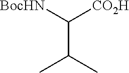 | I-9 | 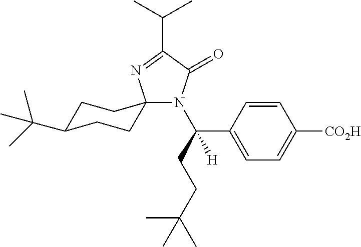 [SMILES: CC(C)C1=NC2(CCC(C(C)(C)C)CC2)N([C@H](CCC(C)(C)C)c2ccc(C(=O)O)cc2)C1=O] |
| 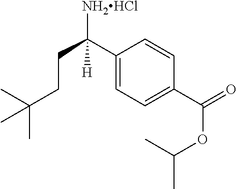 | 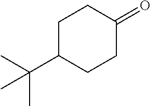 | 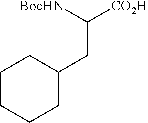 [SMILES: CC(C)(C)OC(=O)NC(CC1CCCCC1)C(=O)O] | I-10 | 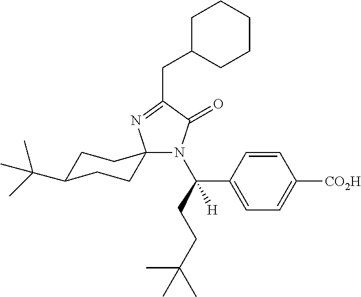 [SMILES: CC(C)(C)CC[C@H](c1ccc(C(=O)O)cc1)N1C(=O)C(CC2CCCCC2)=NC12CCC(C(C)(C)C)CC2] |
| 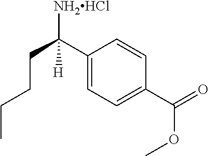 | 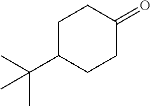 | 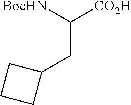 [SMILES: CC(C)(C)OC(=O)NC(CC1CCC1)C(=O)O] | I-11 | 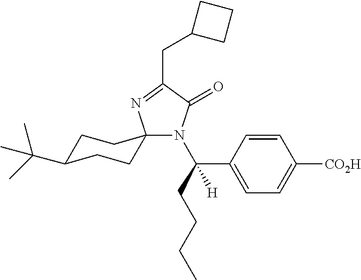 [SMILES: CCCC[C@H](c1ccc(C(=O)O)cc1)N1C(=O)C(CC2CCC2)=NC12CCC(C(C)(C)C)CC2] |
| 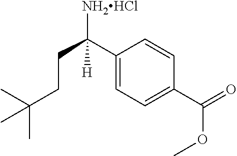 | 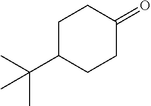 | 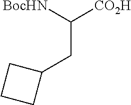 [SMILES: CC(C)(C)OC(=O)NC(CC1CCC1)C(=O)O] | I-12 | 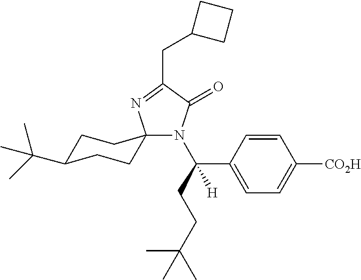 [SMILES: CC(C)(C)CC[C@H](c1ccc(C(=O)O)cc1)N1C(=O)C(CC2CCC2)=NC12CCC(C(C)(C)C)CC2] |
| 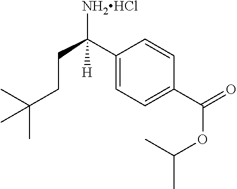 | 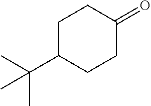 | 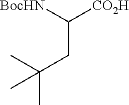 [SMILES: CC(C)(C)CC(NC(=O)OC(C)(C)C)C(=O)O] | I-13 | 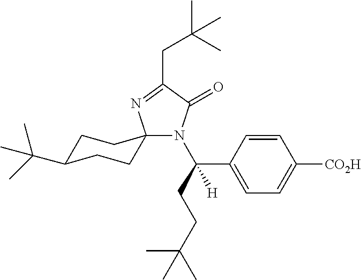 [SMILES: CC(C)(C)CC[C@H](c1ccc(C(=O)O)cc1)N1C(=O)C(CC(C)(C)C)=NC12CCC(C(C)(C)C)CC2] |
| 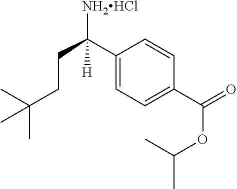 | 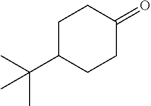 | 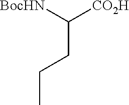 | I-14 | 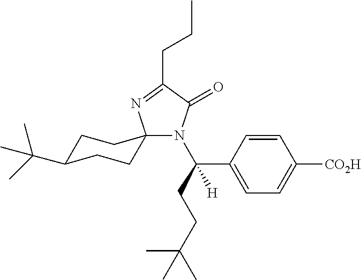 [SMILES: CCCC1=NC2(CCC(C(C)(C)C)CC2)N([C@H](CCC(C)(C)C)c2ccc(C(=O)O)cc2)C1=O] |
| 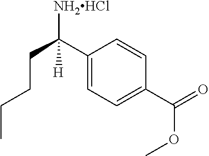 | 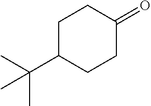 | 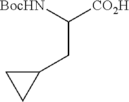 [SMILES: CC(C)(C)OC(=O)NC(CC1CC1)C(=O)O] | I-15 | 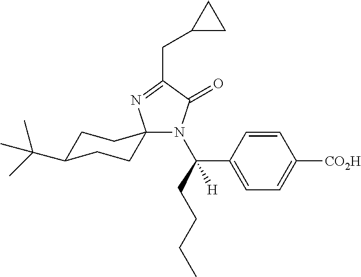 [SMILES: CCCC[C@H](c1ccc(C(=O)O)cc1)N1C(=O)C(CC2CC2)=NC12CCC(C(C)(C)C)CC2] |
| 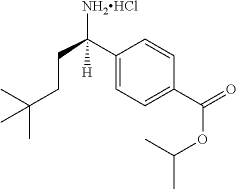 | 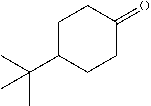 | 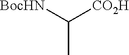 | I-16 | 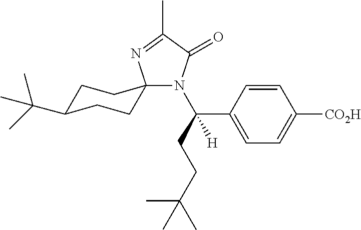 [SMILES: CC1=NC2(CCC(C(C)(C)C)CC2)N([C@H](CCC(C)(C)C)c2ccc(C(=O)O)cc2)C1=O] |
| 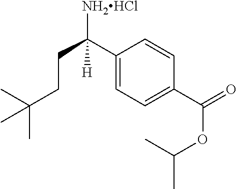 | 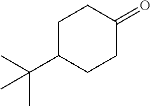 | 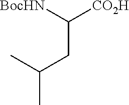 [SMILES: CC(C)CC(NC(=O)OC(C)(C)C)C(=O)O] | I-17 | 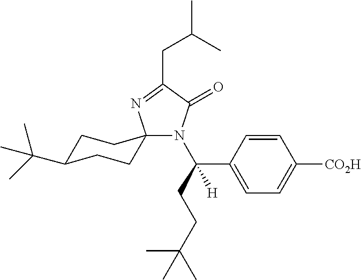 [SMILES: CC(C)CC1=NC2(CCC(C(C)(C)C)CC2)N([C@H](CCC(C)(C)C)c2ccc(C(=O)O)cc2)C1=O] |
| 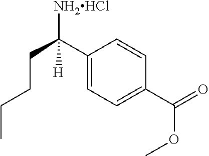 | 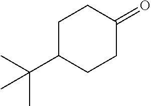 | 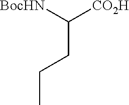 | I-18 | 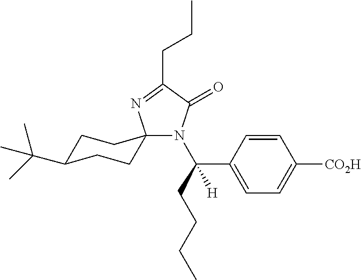 [SMILES: CCCC[C@H](c1ccc(C(=O)O)cc1)N1C(=O)C(CCC)=NC12CCC(C(C)(C)C)CC2] |
| 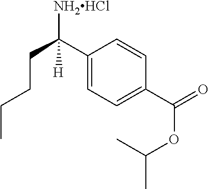 | 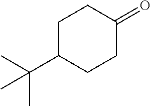 | 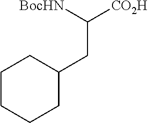 [SMILES: CC(C)(C)OC(=O)NC(CC1CCCCC1)C(=O)O] | I-19 | 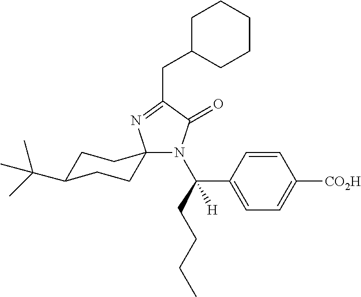 [SMILES: CCCC[C@H](c1ccc(C(=O)O)cc1)N1C(=O)C(CC2CCCCC2)=NC12CCC(C(C)(C)C)CC2] |
| 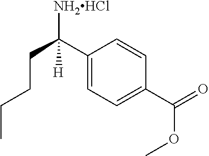 | 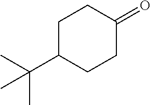 | 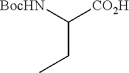 | I-20 | 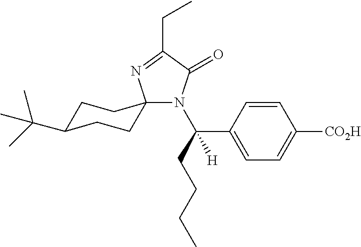 [SMILES: CCCC[C@H](c1ccc(C(=O)O)cc1)N1C(=O)C(CC)=NC12CCC(C(C)(C)C)CC2] |
| 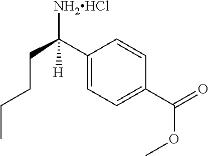 | 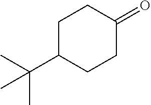 | 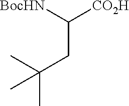 [SMILES: CC(C)(C)CC(NC(=O)OC(C)(C)C)C(=O)O] | I-21 | 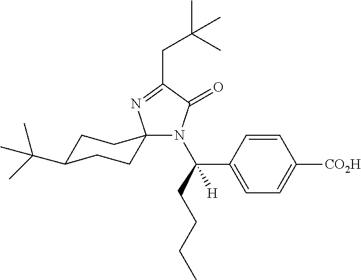 [SMILES: CCCC[C@H](c1ccc(C(=O)O)cc1)N1C(=O)C(CC(C)(C)C)=NC12CCC(C(C)(C)C)CC2] |
| 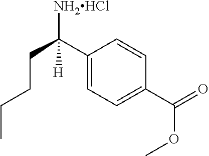 | 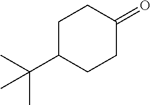 | 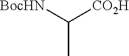 | I-22 | 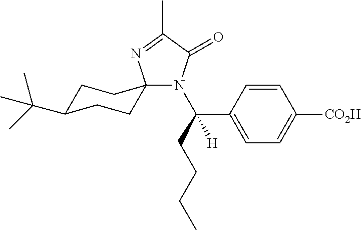 [SMILES: CCCC[C@H](c1ccc(C(=O)O)cc1)N1C(=O)C(C)=NC12CCC(C(C)(C)C)CC2] |

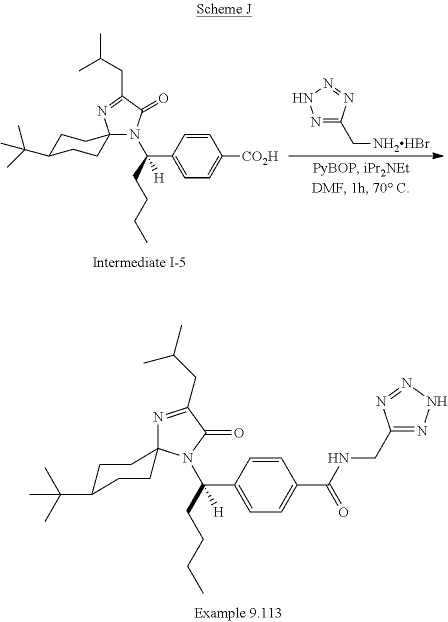

The product from Intermediate I-5 (65 mg, 0.142 mmol, 1 eq), (2H-tetrazol-5-yl)methanamine hydrobromide (38 mg, 1.5 eq), iPr₂NEt (0.076 mL, 3 eq), and PyBOP (89 mg, 1.2 eq) were combined in DMF (3 mL) and were stirred at 70° C. for 1 hour. The solvent was removed in vacuo to afford a crude residue which was purified via reversed-phase C18 chromatography (ISCO, 30 g C-18 Gold column, gradient elution, 30% MeCN in water to 100% MeCN) to afford Example 9.113 (65 mg, 85%).

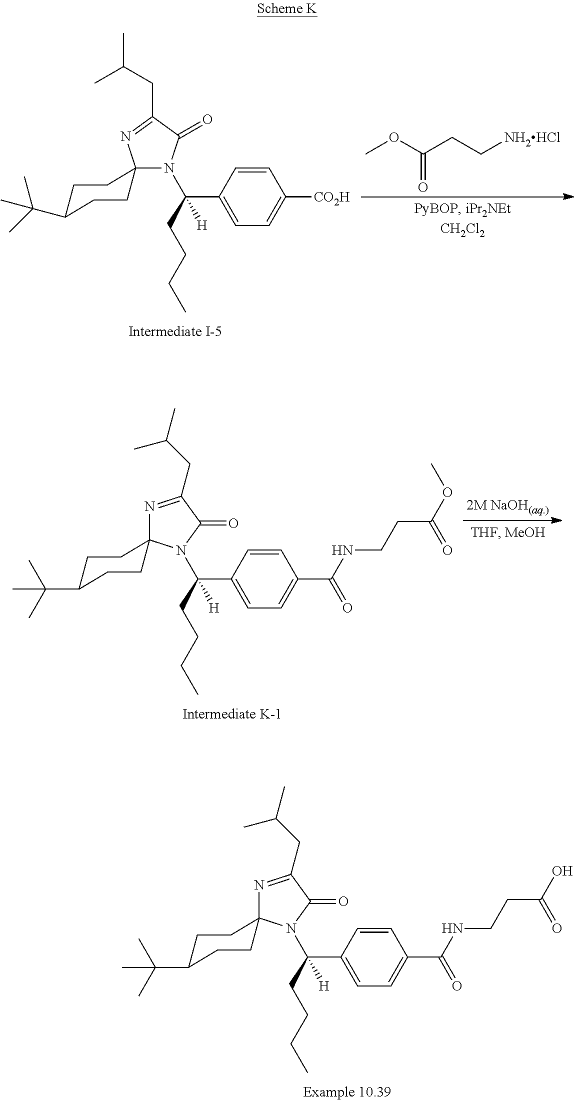

### Step 1

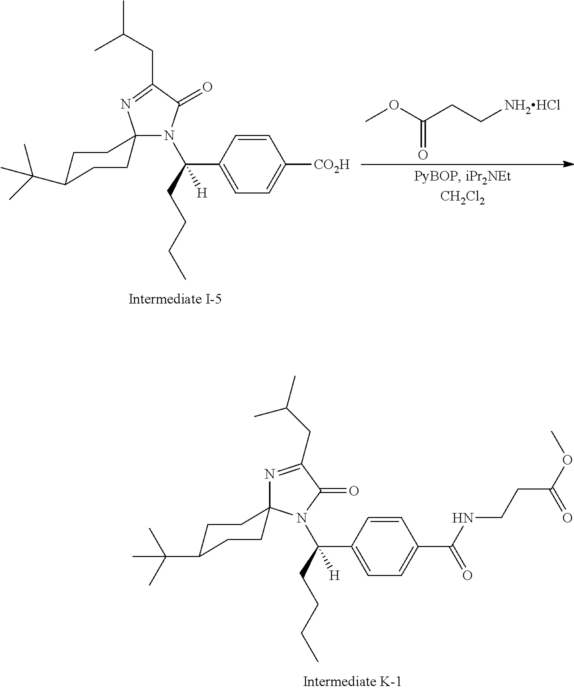

A mixture of Intermediate I-5 (40 mg, 0.088 mmol, 1 eq), PyBOP (55 mg, 1.2 eq), methyl 3-aminopropanoate hydrochloride (16 mg, mmol, 1.3 eq) and iPr₂NEt (0.047 mL, 3.0 eq) in DMF (5 mL) was stirred overnight at room temperature. The reaction mixture was evaporated to afford a crude residue which was purified via silica gel chromatography (ISCO, 12 g column, gradient elution, 0% to 70% EtOAc in hexanes) to provide intermediate K-1 (44 mg, 92%).

### Step 2

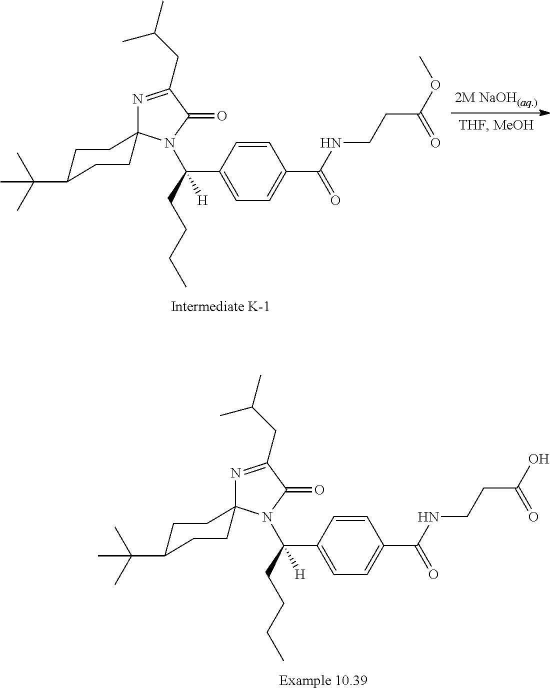 [SMILES: CCCC[C@H](c1ccc(C(=O)NCCC(=O)OC)cc1)N1C(=O)C(CC(C)C)=NC12CCC(C(C)(C)C)CC2 ; CCCC[C@H](c1ccc(C(=O)NCCC(=O)O)cc1)N1C(=O)C(CC(C)C)=NC12CCC(C(C)(C)C)CC2]

Intermediate K-1 (44 mg, 0.081 mmol) was dissolved in THF (8 mL) and MeOH (2 mL). 2M NaOH_{(aq.)} (2 mL) was added and the reaction was stirred at room temperature for 2 h. The reaction mixture was concentrated to ˜⅓ volume and the solution was adjusted to ˜pH3 with 1M HCl_{(aq.)} and the resulting solution was extracted with EtOAc. The combined organic layers were dried over anhydrous Na₂SO₄, filtered, and evaporated to afford a crude residue which was purified via reversed-phase C18 column chromatography (ISCO, 30 g Gold C18 column, gradient elution 30% to 100% MeCN in H₂O) to afford Example 10.39 (35 mg) as a white solid.

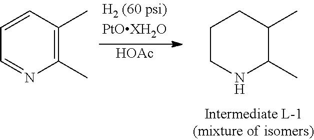

Platinum oxide (300 mg) was added to a solution of 2,3-dimethylpyridine (5 g, 47 mmol) in HOAc (100 mL) in a Parr hydrogenation bottle. The bottle was then pressurized with hydrogen gas to 60 psi, and shaken, refilling with hydrogen to 60 psi until the uptake of hydrogen ceased (−24 h). The reaction was then purged with nitrogen, filtered through Celite, and concentrated. The resulting residue was dissolved in water and the solution was made basic with 40% NaOH_{(aq.)}. The solution was extracted with Et₂O. The combined organic layers were dried over anhydrous Na₂SO₄, filtered, and evaporated to afford Intermediate L-1 (2 g) as a mixture of isomers.

TABLE 7
Using the requisite starting material, and a method similar to that outlined
in Scheme L, the following compound was prepared:

| | | Intermediate |
|---|---|---|
| Starting Material | Intermediate Number | Structure |
| 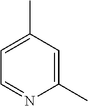 | L-2 | 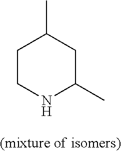 |

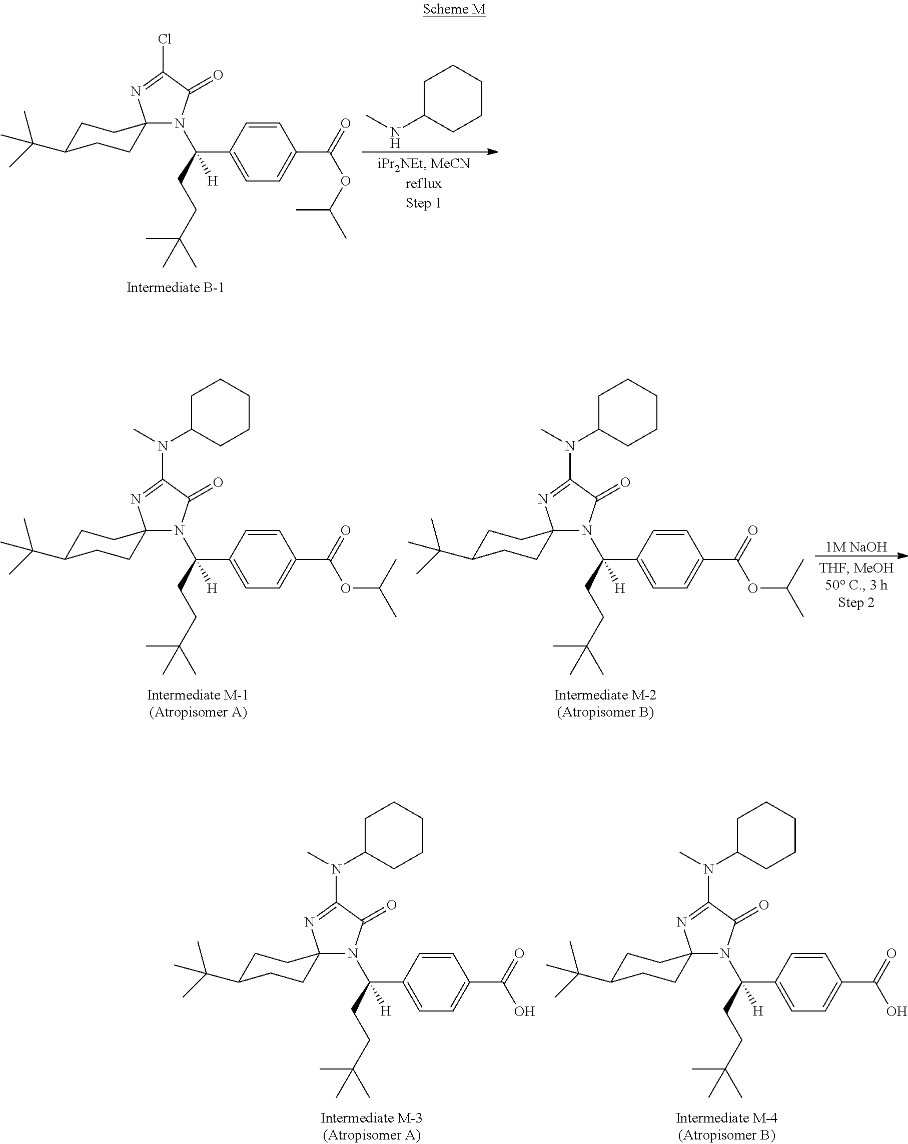

### Step 1

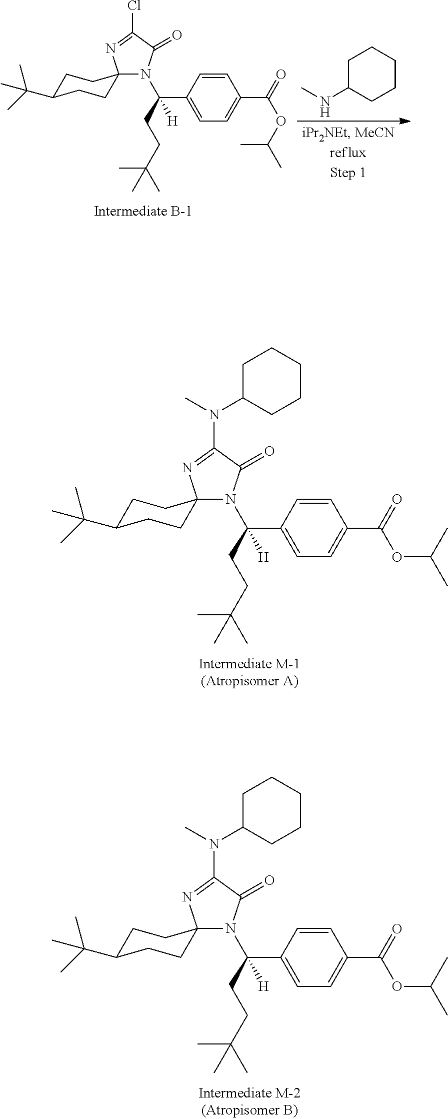

Intermediate B-1 (80 mg, 0.16 mmol, 1 eq), N-methylcyclohexanamine (0.063 mL, 0.48 mmol, 3 eq), and iPr₂NEt (0.083 mL, 0.48 mmol, 3 eq) were dissolved in acetonitrile (5 mL). The reaction was heated for 16 h at reflux. A second portion of both N-methylcyclohexanamine (0.063 mL, 0.48 mmol, 3 eq), and iPr₂NEt (0.083 mL, 0.48 mmol, 3 eq) were added and refluxing was continued for 48 h. After cooling the reaction to room temperature, the reaction was concentrated, and the resulting residue was subjected to preparative thin-layer chromatography (4:1 hexanes:EtOAc) to afford an inseparable mixture of Intermediate M-1 (Atropisomer A) and Intermediate M-2 (Atropisomer B) (35 mg).

### Step 2

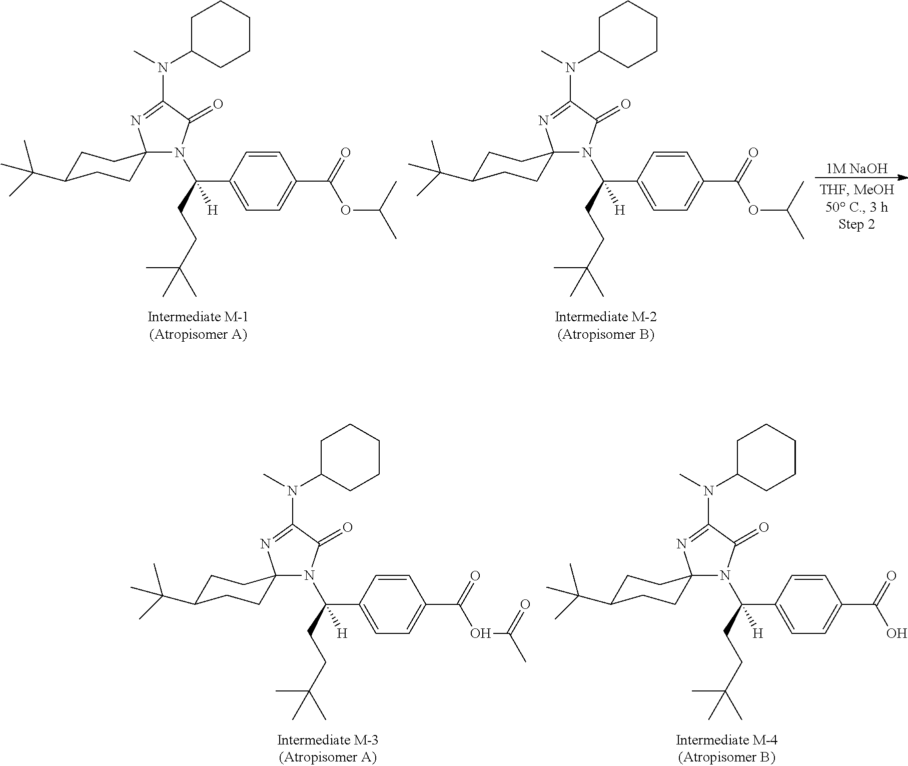

A solution of Intermediate M-1 (Atropisomer A) and Intermediate M-2 (Atropisomer B) (35 mg, 0.26 mmol) in THF (3 mL) and MeOH (4 mL) was treated with 1M NaOH (aq.) (1 mL). The reaction mixture was heated with stirring for 3 h at 50° C. After cooling to room temperature, the reaction mixture was stirred overnight. The reaction was concentrated and partitioned between EtOAc and 1M HCl (aq.). The aqueous layer was discarded, and the organic layer was dried over anhydrous Na₂SO₄, filtered and evaporated to afford an inseparable mixture of Intermediate M-3 (Atropisomer A) and Intermediate M-4 (Atropisomer B) (31 mg), which was used in the next step without further purification.

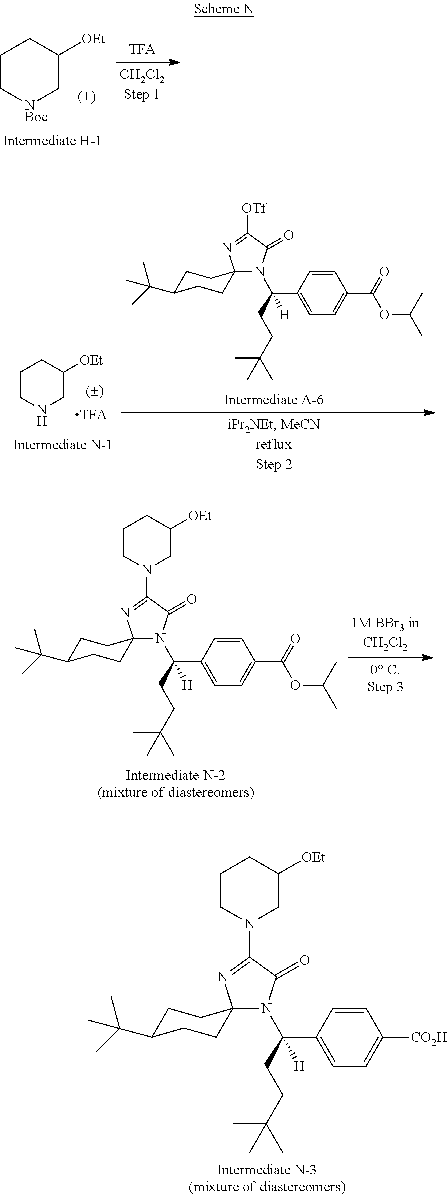

### Step 1

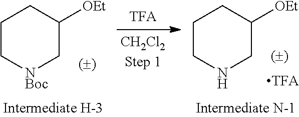 [SMILES: CCOC1CCCN(C(=O)OC(C)(C)C)C1 ; CCOC1CCCNC1]

A solution of Intermediate H-1 (105 mg, 0.46 mmol) in CH₂Cl₂ (1 mL) was treated with TFA (0.5 mL). The resulting mixture was stirred at room temperature for 2 h then was concentrated to afford Intermediate N-1, which was used in the subsequent step without further purification.

### Step 2

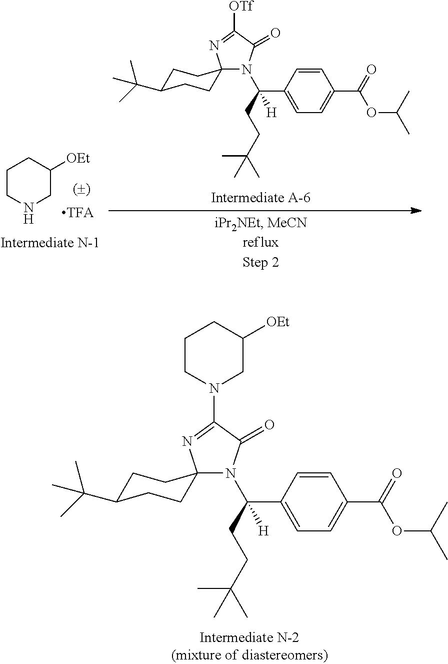

A solution of Intermediate A-6 (91 mg, 0.15 mmol, 1 eq), Intermediate N-1 (0.46 mmol, 3 eq), and iPr₂NEt (77 mg, 0.59 mmol, 4 eq) in MeCN (2 mL) was heated at reflux for 1 h. The reaction was concentrated and the resulting residue purified via silica gel chromatography (gradient elution, 0% to 10% EtOAc in hexanes) to afford Intermediate N-2 (76 mg, 86%, mixture of diastereomers).

### Step 3

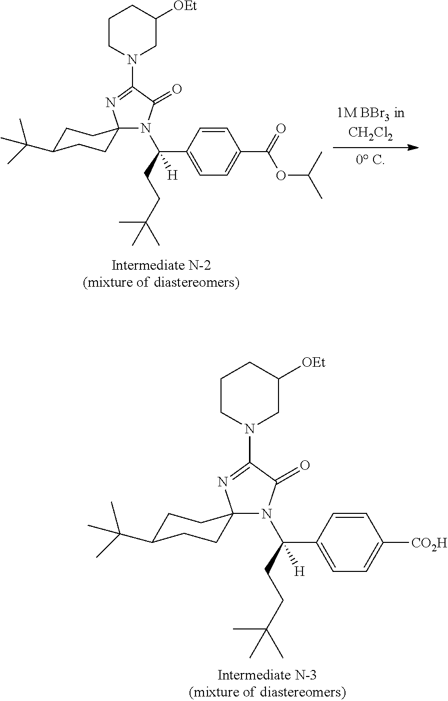

A solution of Intermediate N-2 (76 mg, 0.13 mmol, 1 eq) in CH₂Cl₂ (3 mL) at 0° C. was treated with 1M BBr₃ in CH₂Cl₂ (0.64 mL, 0.64 mmol, 5 eq). The reaction was stirred for 2 h at 0° C. The reaction was quenched with water. After partitioning between EtOAc and brine, the aqueous layer was removed. The organic layer was dried over anhydrous Na₂SO₄, filtered, and evaporated to afford Intermediate N-3 (70 mg, quant., mixture of diastereomers) as a thick oil which was used in the next step without further purification.

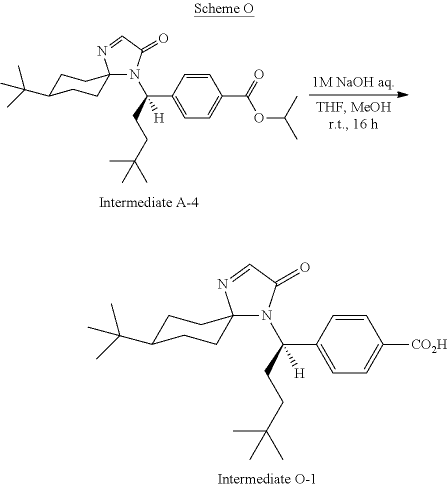

Intermediate A-4 (193 mg, 0.41 mmol) was dissolved in MeOH (2.5 mL) and THF (5 mL). Addition of 1M NaOH_{(aq.)} (2.5 mL) was followed by stirring overnight at room temperature. The reaction was partitioned between EtOAc and 1N HCl_{(aq.)}. The aqueous layer was discarded and the organic layer was washed with brine, dried over anhydrous Na₂SO₄, filtered, and evaporated to afford intermediate 0-1 (190 mg, quant.) which was used in the next step without further purification,

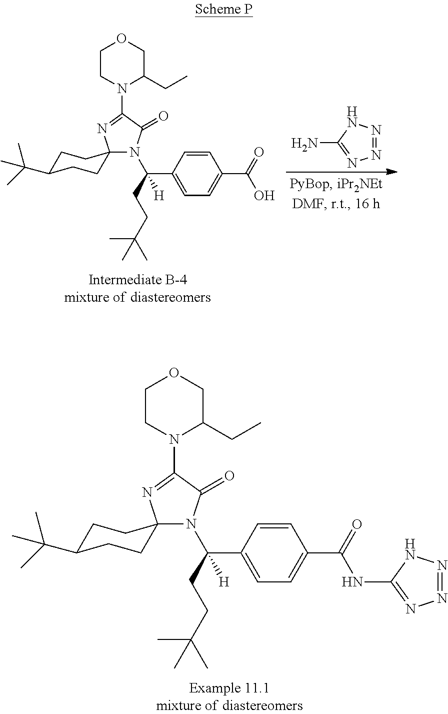 [SMILES: CCC1COCCN1C1=NC2(CCC(C(C)(C)C)CC2)N([C@H](CCC(C)(C)C)c2ccc(C(=O)O)cc2)C1=O ; Nc1nnn[nH]1 ; CCC1COCCN1C1=NC2(CCC(C(C)(C)C)CC2)N([C@H](CCC(C)(C)C)c2ccc(C(=O)Nc3nnn[nH]3)cc2)C1=O]

Intermediate B-4 (60 mg, 0.1 mmol, 1 eq), 1H-tetrazol-5-amine (14 mg, 0.16 mmol, 1.5 eq), iPr₂NEt (0.057 mL, 0.32 mmol, 3 eq), and PyBOP (68 mg, 0.13 mmol, 1.2 eq) were combined in DMF (2 mL) and were stirred at room temperature for 16 hours. The crude reaction mixture was directly purified via reversed-phase C18 chromatography (Biotage SP-1, 16 g Analogix C18 column, gradient elution, 10% MeCN in water with 0.1% HCOOH to 100% MeCN with 0.1% HCOOH) to afford Example 11.1 (30 mg, 40% yield) as a mixture of diastereomers.

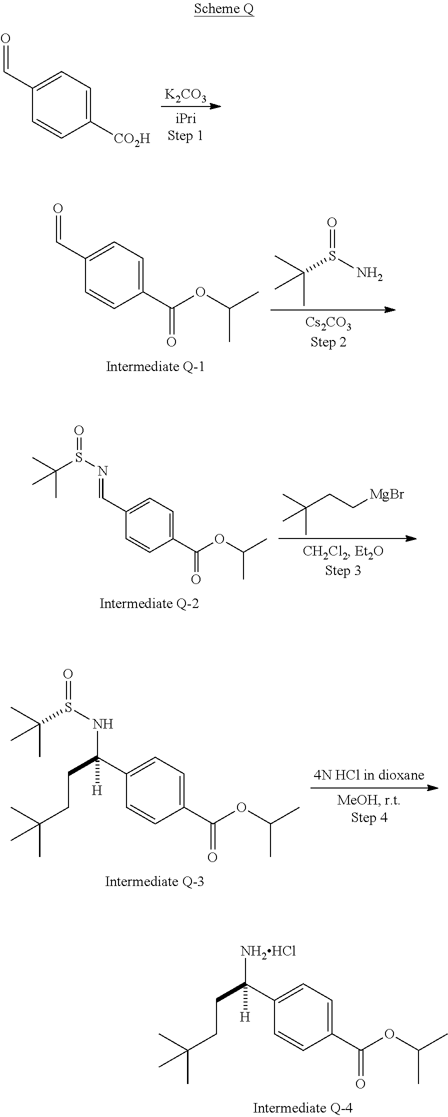

### Step 1

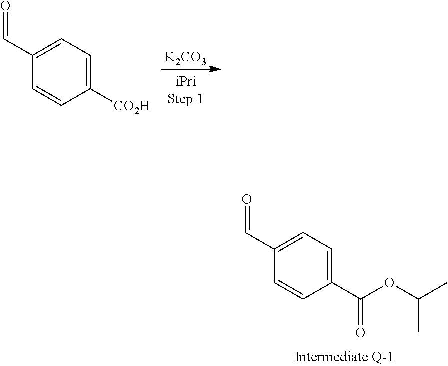

Isopropyl iodide (68 g, 399 mmol), 4-formylbenzoic acid (20 g, 133 mmol), and K₂CO₃ (37 g, 266 mmol) were taken up in THF/DMF (2/1, 300 ml), and the mixture was heated at 70° C. for 64 h. The solution was partitioned between EtOAc and water. The aqueous layer was extracted with EtOAc. The combined organic layers were washed with brine and dried (MgSO₄). The solution was filtered and concentrated which yielded 20.3 g (79%) of Intermediate Q-1 as an oil that solidified upon standing.

### Step 2

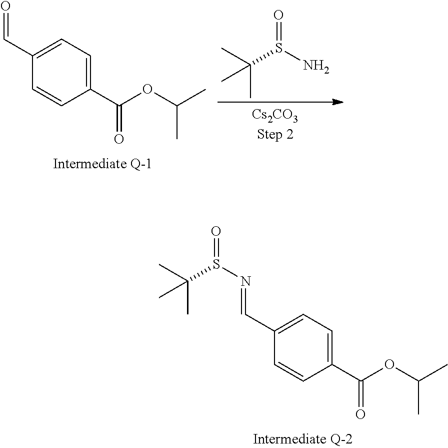

Intermediate Q-1 (21.2 g, 110 mmol), (S)-2-methylpropane-2-sulfinamide (13.4 g, 110 mmol), and Cs₂CO₃ (36 g. 110 mmol) were taken up in DCM (400 ml), and the mixture was stirred at 42° C. for 30 h. The solution was filtered and concentrated. This yielded 32.2 g (99%) of Intermediate Q-2 as an oil that solidified upon standing.

### Step 3

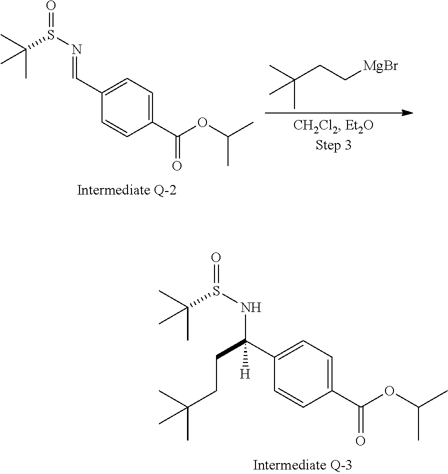

The grignard reagent was made as follows: Magnesium turnings (2.4 g, 100 mmol) were suspended in dry Et₂O (150 ml) under N₂. A few iodine crystals were added to the mixture. The 1-bromo-3,3-diemthyl butane (16.5 g, 100 mmol) in Et₂O (50 ml) was added in portions over ˜45 minutes to maintain gentle reflux. After the addition of all of the 1-bromo-3,3-diemthyl butane, the reaction was refluxed for 2 hr. The gringnard solution was used as is in the next step.

The grignard reagent (100 mmol in 200 ml of Et₂O) was added to a solution of Intermediate Q-2 (9.9 g, 33.5 mmol) at −78° C. The solution was slowly warmed to RT. After stirring at RT for 2 h, the reaction was quenched with sat. NH₄Cl_{(aq.)} at 0° C. Ethyl acetate was added, and the mixture was stirred at RT for 1 h. The layers were separated, and the aqueous layer was extracted with EtOAc. The combined organic layers were washed with brine and dried (MgSO₄). The mixture was filtered and concentrated. The residue was purified via gradient flash chromatography (0-40% EtOAc in hexanes, SiO₂). The major fraction was recrystallized from heptane/IPA to yield 2.8 g Intermediate Q-3. The mother liquor was concentrated to afford a residue which was recrystallized from heptane/IPA to provide an additional 1.3 g (32% total) of Intermediate Q-3.

### Step 4

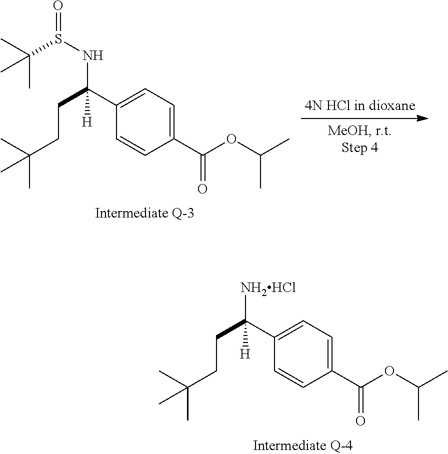

Intermediate Q-3 (3.18 g, 8.3 mmol) was taken up in MeOH (30 ml), and 4 M HCl in dioxane (4.1 ml) was added at RT. The solution was stirred at RT for 1.5 h. The solution was concentrated, and ether was added which resulted in the formation of a white solid. The solid was collected and rinsed with ether. The solid was dried to provide 2.2 g (84%) of Intermediate Q-4.

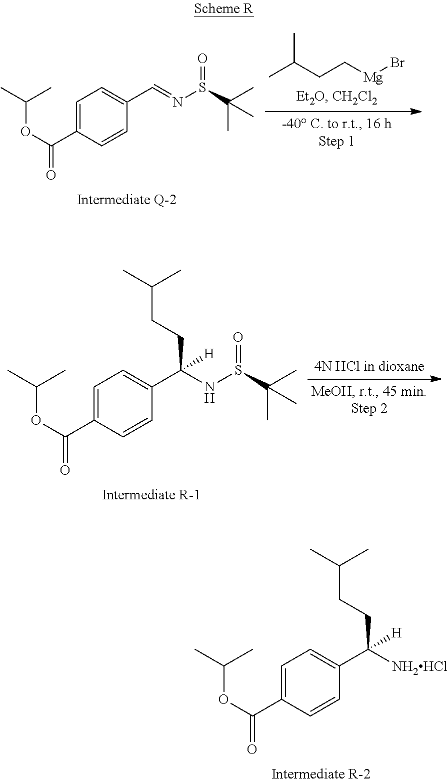

### Step 1

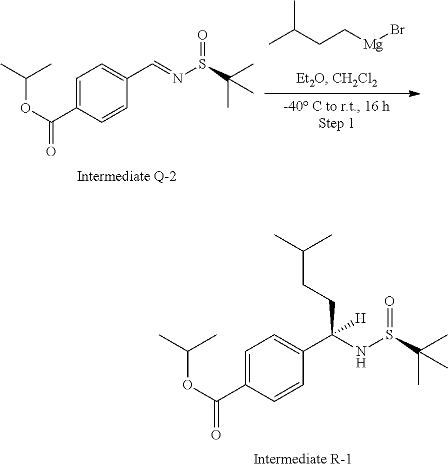

Magnesium turnings (14.6 g, 600 mmol, 1 eq) were added to Et₂O (400 mL) under a nitrogen atmosphere in a round bottomed flask with a reflux condenser attached. A crystal of iodine was added to the mixture, followed by 1-bromo-3-methylbutane (20 mL). The mixture was gently warmed to 30° C., at which point the reaction initiated and a vigorous refluxing ensued. Additional aliquots of 1-bromo-3-methylbutane were added at a rate such that the refluxing was maintained. After completion of the addition of 1-bromo-3-methylbutane (total amount: 72 mL, 601.1 mmol, 1 eq), the mixture was refluxed for 2 h. The reaction was then cooled to room temperature, affording the requisite isopentylmagnesium bromide solution.

Intermediate Q-2 (90.0 g, 305 mmol, 1.00 eq) was dissolved in CH₂Cl₂ (1000 mL), and the solution was cooled to −40° C. The previously prepared isopentylmagnesium bromide solution was added dropwise over a one hour period via a dropping funnel to the sulfinimine solution. The reaction was stirred at −40° C. for 4 h. The reaction was stirred for an additional 16 h, during which time the cold bath was allowed to expire. Saturated ammonium chloride_{(aq.)} was added to the reaction and the resulting murky suspension was stirred for 30 min. An attempt to filter the reaction through Celite® resulted in a clogged filter pad. The crude reaction, including the clogged Celite® pad was transferred to an Erlenmeyer flask. EtOAc (2000 mL) and 20% sodium citrate_{(aq.)} (2000 mL) were added to the crude mixture and the solution was stirred for 2 h. The biphasic solution was filtered, and the Celite® left behind in the filter was washed with EtOAc and water. The combined biphasic filtrate was separated. The aqueous layer was extracted with EtOAc. The organic layers were combined, washed with brine twice, dried over anhydrous MgSO₄, filtered, and evaporated to afford a viscous green oil. Silica chromatography (performed in two batches, each on a 600 g silica gel column, gradient elution, 0% to 100% EtOAc in hexanes, SiO₂) afforded the desired addition product as a 5.6:1 mixture of diastereomers. The latter fractions of the product peak were collected separately, as they were enriched in the major diastereomer. The enriched material was recrystallized from hot hexanes to afford the major diastereomer (Intermediate R-1, 9.71 g, 99.8:0.1 dr, ChiralPak AD, 95:5 hexanes:isopropanol, 1 mL/min, 254 nm) as white crystals. Additional crops of crystals can be obtained from the mixed fractions.

### Step 2

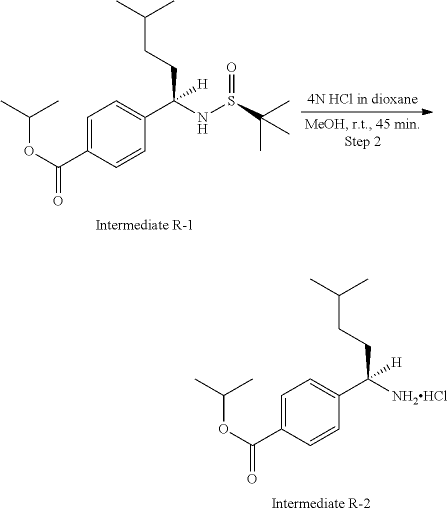

A solution of Intermediate R-1 (22.2 g) in MeOH (100 mL) at room temperature was treated with 4N HCl in dioxane (28 mL). The resulting solution was stirred for 45 min at room temperature. The reaction was concentrated and treated with Et₂O (500 mL) to afford a white solid, which was collected via filtration, washed with Et₂O and dried under vacuum to afford Intermediate R-2 as a white solid (14.7 g).

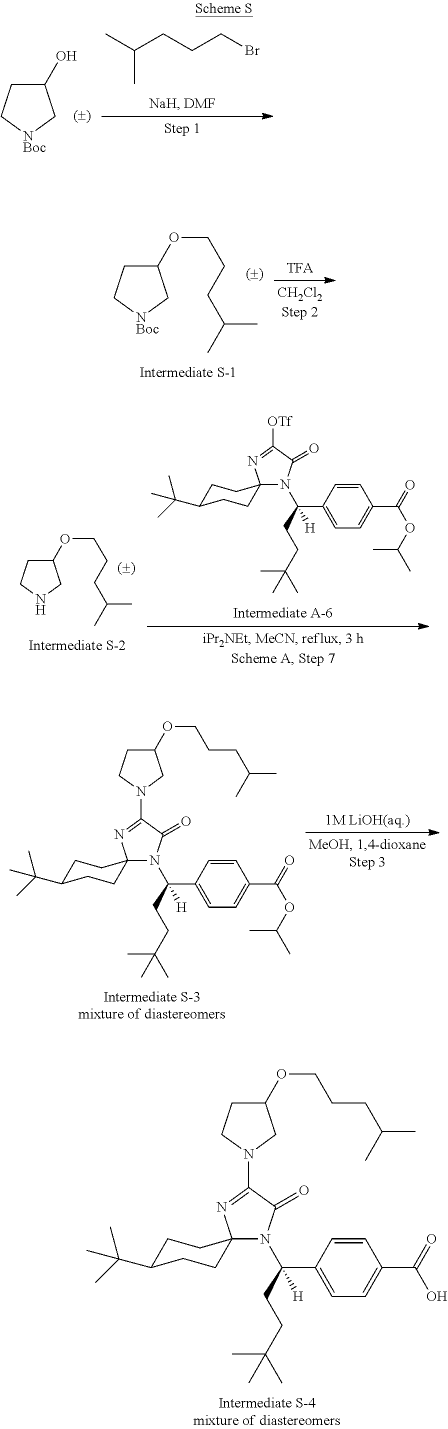

### Step 1

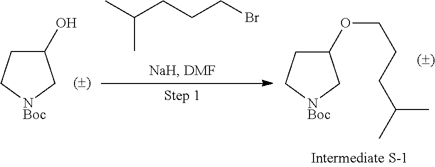

A solution of (±)-tert-butyl 3-hydroxypyrrolidine-1-carboxylate (2.0 g, 10.7 mmol, 1 eq), in DMF (20 mL) was added dropwise to a suspension of NaH (60% w/w dispersion in mineral oil, 0.64 g, 16.0 mmol, 1.5 eq) in DMF (10 mL) at 0° C. The reaction mixture was allowed to warm to room temperature and was stirred for 1 hour. To the reaction was added 1-bromo-4-methylpentane (2.64 g, 16.0 mmol, 1.5 eq). The reaction mixture was stirred 3 h at room temperature. The reaction was concentrated and the resulting residue was partitioned between EtOAc and water. The aqueous layer was discarded and the organic layer was evaporated to afford Intermediate S-1 (2.78 g), which was used in the next step without further purification.

### Step 2

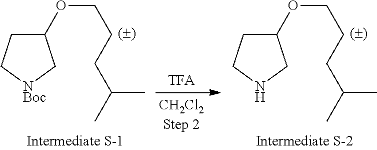 [SMILES: CC(C)CCCOC1CCN(C(=O)OC(C)(C)C)C1 ; CC(C)CCCOC1CCNC1]

At room temperature, trifluoroacetic acid (11 g, 97.0 mmol, 10 eq) was added dropwise to a solution of Intermediate S-1 (2.78 g, 9.70 mmol, 1 eq) in CH₂Cl₂ (40 mL). The resulting reaction mixture was stirred overnight. The reaction was poured into EtOAc/water and the aqueous layer was adjusted to ˜pH 10 with 5% NaOH(aq.). After separating the biphasic solution, the organic layer was washed with water twice and evaporated to afford Intermediate S-2 (1.34 g) which was used in the next step without further purification.

### Step 3

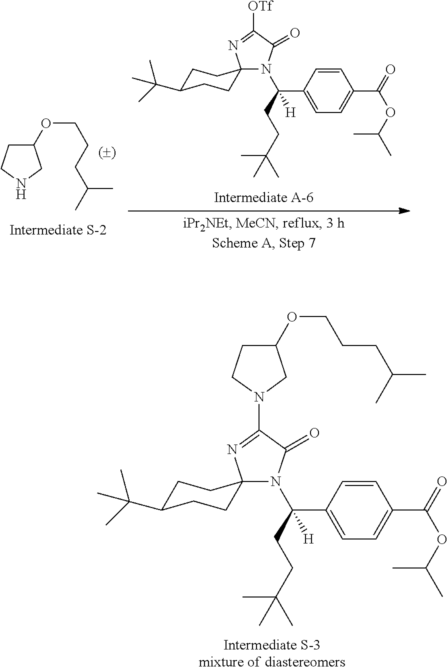 [SMILES: CC(C)CCCOC1CCNC1 ; CC(C)OC(=O)c1ccc([C@@H](CCC(C)(C)C)N2C(=O)C(OS(=O)(=O)C(F)(F)F)=NC23CCC(C(C)(C)C)CC3)cc1 ; CC(C)CCCOC1CCN(C2=NC3(CCC(C(C)(C)C)CC3)N([C@H](CCC(C)(C)C)c3ccc(C(=O)OC(C)C)cc3)C2=O)C1]

Utilizing a method similar to that outlined in Scheme A, Step 7, Intermediate S-2 and Intermediate A-6 were combined to provide Intermediate S-3 as a mixture of diastereomers.

### Step 4

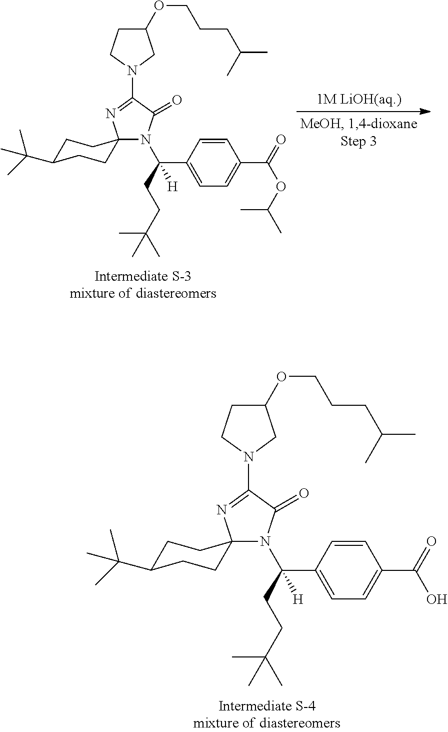

Intermediate S-3 (70 mg, 0.11 mmol) was dissolved in MeOH (2 mL) and 1,4-dioxane (4 mL). Addition of 1M LiOH_{(aq.)} (1.1 mL) was followed by stirring overnight at room temperature. The reaction was partitioned between EtOAc and 1N HCl_{(aq.)}. The aqueous layer was discarded and the organic layer was washed with brine, dried over anhydrous Na₂SO₄, filtered, and evaporated to afford Intermediate S-4 (63 mg, mixture of diastereomers) which was used in the next step without further purification.

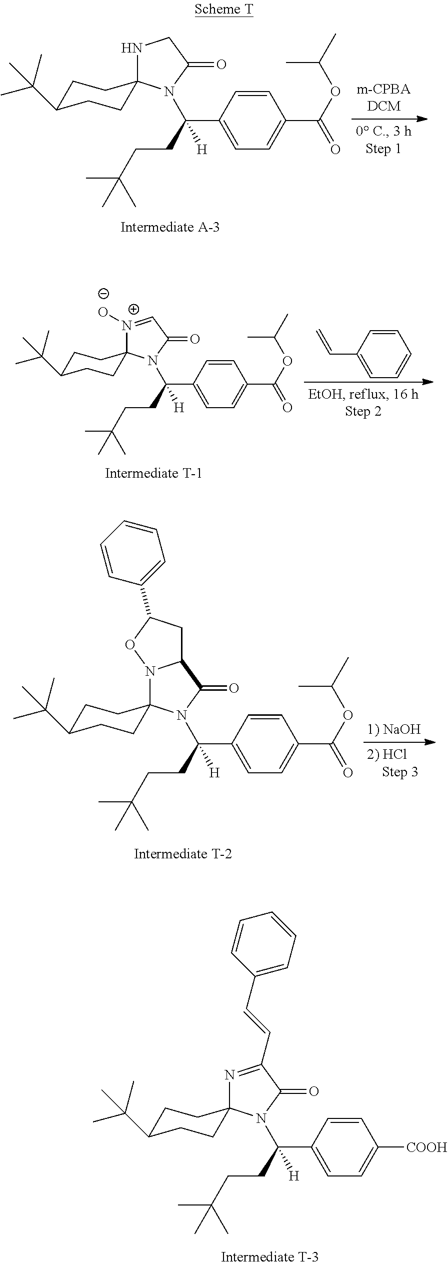

### Step 1:

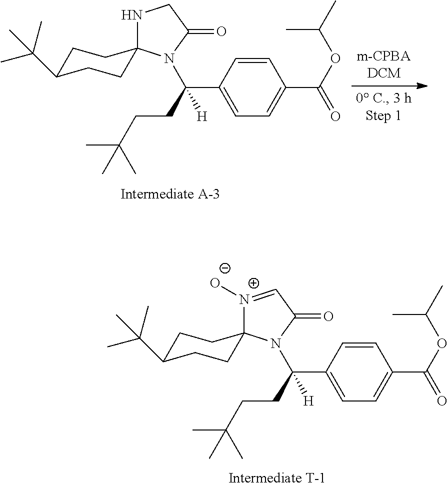 [SMILES: CC(C)OC(=O)c1ccc([C@@H](CCC(C)(C)C)N2C(=O)CNC23CCC(C(C)(C)C)CC3)cc1 ; CC(C)OC(=O)c1ccc([C@@H](CCC(C)(C)C)N2C(=O)C=[N+]([O-])C23CCC(C(C)(C)C)CC3)cc1]

To a 25 mL round flask was added Intermediate A-3 (1.90 g, 4.04 mmol) and dichloromethane (15 mL). The solution was cooled to 0° C. and m-CPBA (2.09 g, 8.48 mmol, 70% purity) was added in one portion. The reaction was stirred at 0° C. for 3 hours. After completion of the reaction, 10% aqueous sodium thiosulfate (5 mL) was added and the mixture was stirred for 10 min. Saturated aqueous NaHCO₃ was added and the mixture was stirred until both phases went clear. The organic layer was separated, and the aqueous layer was extracted twice with DCM. The combined organic layers were washed with saturated NaHCO_{3(aq.)} and brine. The organic layer was dried over anhydrous Na₂SO₄, filtered and concentrated. The resulting residue was chromatographed through a short column of SiO₂ (EtOAc/hexane ½) to afford Intermediate T-1 (628 mg, 32% yield) as a white solid.

### Step 2:

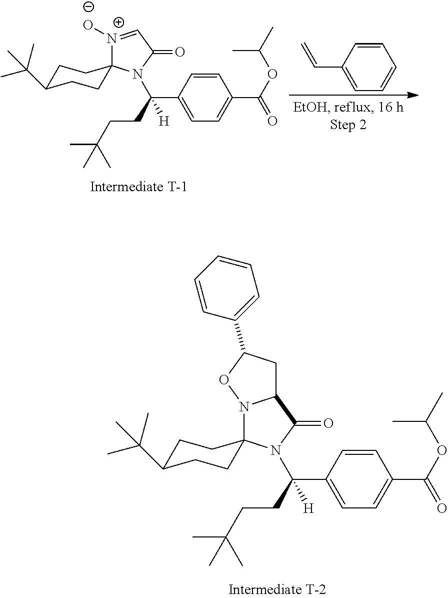

A solution of Intermediate T-1 (113 mg, 0.23 mmol) and styrene (97 mg, 0.93 mmol) in EtOH (5 mL) in a sealed vial was heated at reflux overnight (16 h). The reaction was cooled to rt and concentrated. The residue was chromatographed through a short column of SiO₂ (0-40% EtOAc/hexane) to give the desired product as colorless foam (127 mg, 94% yield).

### Step 3:

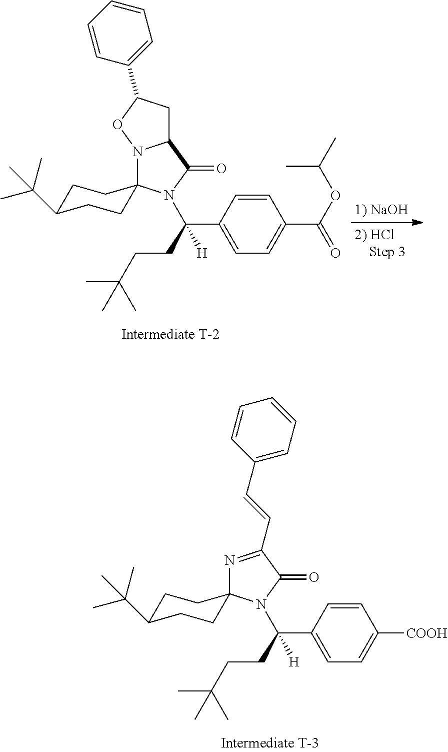

Intermediate T-2 (100 mg, 0.18 mmol) was taken up in 1N NaOH_{(aq.)}/THF/MeOH [1/1/1, 15 mL], and the solution was stirred at room temperature overnight. The solution was concentrated. The residue was partitioned between DCM and 1M HCl_{(aq.)}. The mixture was stirred at room temperature for 0.5 h. The layers were separated, and the aqueous layer was extracted with DCM. The combined organic layers were dried (anhydrous Na₂SO₄), filtered, and concentrated to afford Intermediate T-3 (69 mg, 73% yield).

TABLE 8
Using the requisite starting material, and a method similar to that outlined in
Scheme T the following compounds were prepared:

| | | Inter- | |
|---|---|---|---|
| | | medi- | |
| | Styrene | ate | |
| Cyclization Protuct | Used in | Num- | |
| Used in Step 1 | Step 2 | ber | Intermediate Prepared |
| 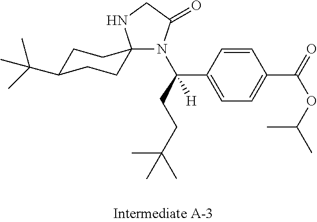 [SMILES: CC(C)OC(=O)c1ccc([C@@H](CCC(C)(C)C)N2C(=O)CNC23CCC(C(C)(C)C)CC3)cc1] | 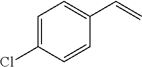 | T-4 | 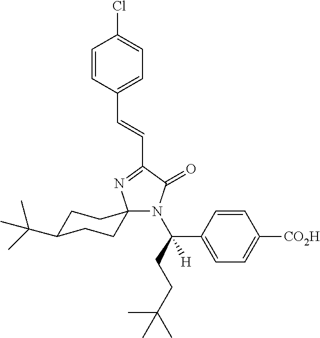 [SMILES: CC(C)(C)CC[C@H](c1ccc(C(=O)O)cc1)N1C(=O)C(/C=C/c2ccc(Cl)cc2)=NC12CCC(C(C)(C)C)CC2] |
| 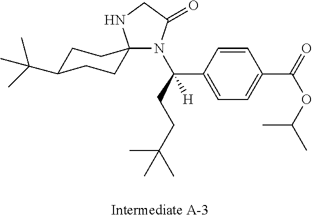 [SMILES: CC(C)OC(=O)c1ccc([C@@H](CCC(C)(C)C)N2C(=O)CNC23CCC(C(C)(C)C)CC3)cc1] | 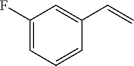 | T-5 | 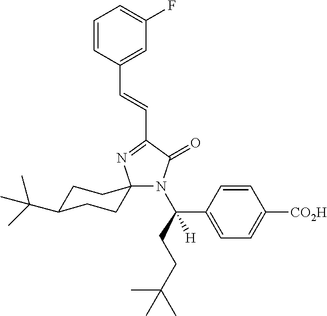 [SMILES: CC(C)(C)CC[C@H](c1ccc(C(=O)O)cc1)N1C(=O)C(/C=C/c2cccc(F)c2)=NC12CCC(C(C)(C)C)CC2] |
| 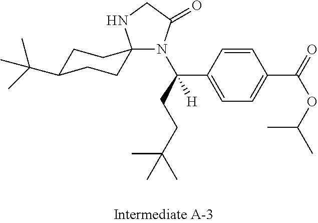 [SMILES: CC(C)OC(=O)c1ccc([C@@H](CCC(C)(C)C)N2C(=O)CNC23CCC(C(C)(C)C)CC3)cc1] | 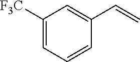 | T-6 | 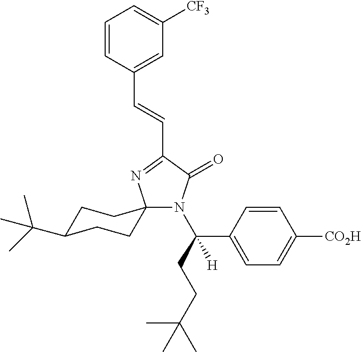 [SMILES: CC(C)(C)CC[C@H](c1ccc(C(=O)O)cc1)N1C(=O)C(/C=C/c2cccc(C(F)(F)F)c2)=NC12CCC(C(C)(C)C)CC2] |

TABLE 9
Using the requisite starting material, and a method similar to that outlined in
one of the following: Scheme D, Scheme G, or Scheme J the following compounds
were prepared:

| | | | LCMS | | |
|---|---|---|---|---|---|
| Benzoic | | | | Ret | |
| Acid | Ex. | Structure | LC | (min) | [M + H]⁺ |
| A-10 | 9.1 | 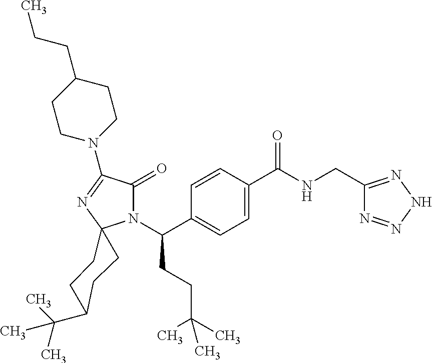 [SMILES: CCCC1CCN(C2=NC3(CCC(C(C)(C)C)CC3)N([C@H](CCC(C)(C)C)c3ccc(C(=O)NCc4nn[nH]n4)cc3)C2=O)CC1] | 5 | 18.52 (20) | 633 |
| A-11 | 9.2 | 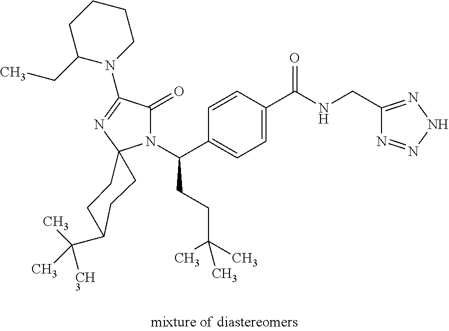 [SMILES: CCC1CCCCN1C1=NC2(CCC(C(C)(C)C)CC2)N([C@H](CCC(C)(C)C)c2ccc(C(=O)NCc3nn[nH]n3)cc2)C1=O] | 5 | 17.63 (20) | 619 |
| A-12 | 9.3 | 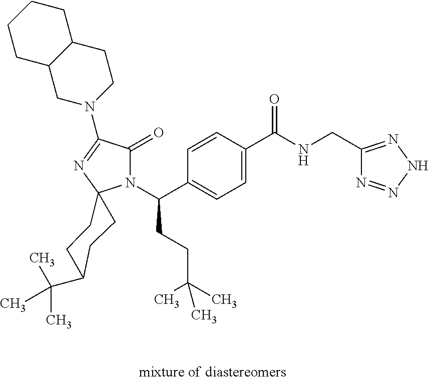 [SMILES: CC(C)(C)CC[C@H](c1ccc(C(=O)NCc2nn[nH]n2)cc1)N1C(=O)C(N2CCC3CCCCC3C2)=NC12CCC(C(C)(C)C)CC2] | 5 | 18.28 18.37 18.53 (20) | 645 |
| A-13 | 9.4 | 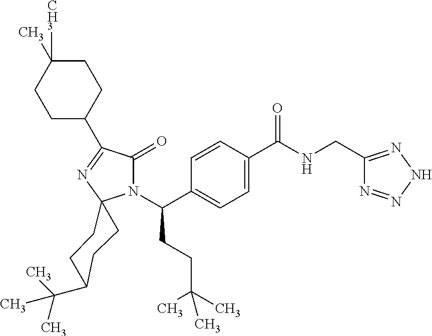 [SMILES: CC(C)(C)CC[C@H](c1ccc(C(=O)NCc2nn[nH]n2)cc1)N1C(=O)C(C2CCC(C)(C)CC2)=NC12CCC(C(C)(C)C)CC2] | 5 | 13.91 (15) | 619 |
| A-14 | 9.5 | 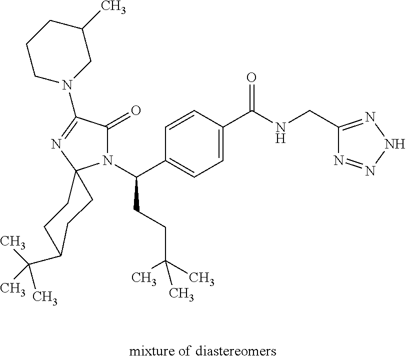 [SMILES: CC1CCCN(C2=NC3(CCC(C(C)(C)C)CC3)N([C@H](CCC(C)(C)C)c3ccc(C(=O)NCc4nn[nH]n4)cc3)C2=O)C1] | 5 | 17.20 (20) | 605 |
| M-3 and M-4 mixture | 9.6 | 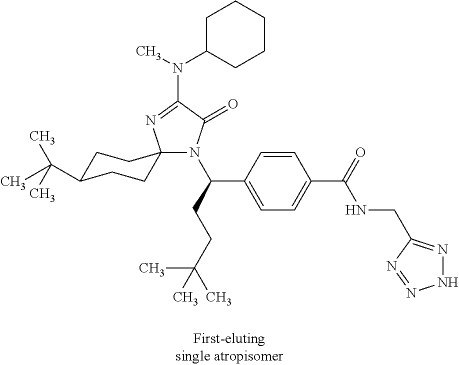 [SMILES: CN(C1=NC2(CCC(C(C)(C)C)CC2)N([C@H](CCC(C)(C)C)c2ccc(C(=O)NCc3nn[nH]n3)cc2)C1=O)C1CCCCC1] | 1 | 4.44 | 619 |
| M-3 and M-4 mixture | 9.7 | 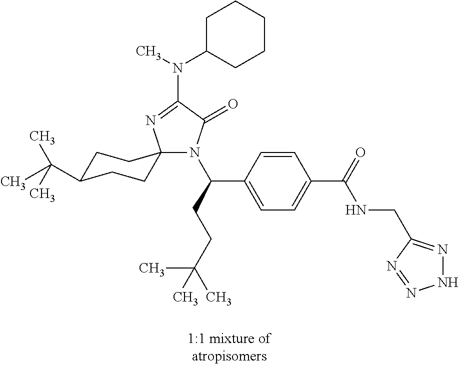 [SMILES: CN(C1=NC2(CCC(C(C)(C)C)CC2)N([C@H](CCC(C)(C)C)c2ccc(C(=O)NCc3nn[nH]n3)cc2)C1=O)C1CCCCC1] | 1 | 4.39 4.44 | 619 |
| A-8 | 9.8 | 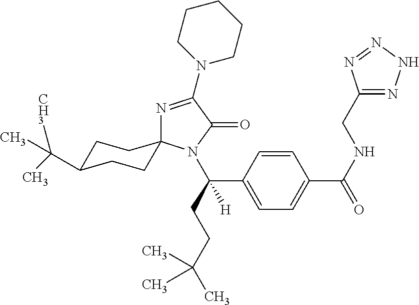 [SMILES: CC(C)(C)CC[C@H](c1ccc(C(=O)NCc2nn[nH]n2)cc1)N1C(=O)C(N2CCCCC2)=NC12CCC(C(C)(C)C)CC2] | 1 | 2.13 | 592 |
| A-17 | 9.9 | 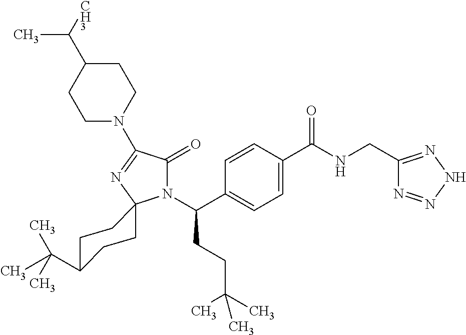 [SMILES: CC(C)C1CCN(C2=NC3(CCC(C(C)(C)C)CC3)N([C@H](CCC(C)(C)C)c3ccc(C(=O)NCc4nn[nH]n4)cc3)C2=O)CC1] | 5 | 14.43 (15) | 633 |
| A-18 | 9.10 | 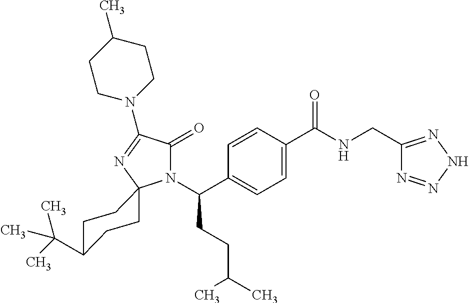 [SMILES: CC(C)CC[C@H](c1ccc(C(=O)NCc2nn[nH]n2)cc1)N1C(=O)C(N2CCC(C)CC2)=NC12CCC(C(C)(C)C)CC2] | 5 | 13.32 (15) | 591 |
| A-20 | 9.11 | 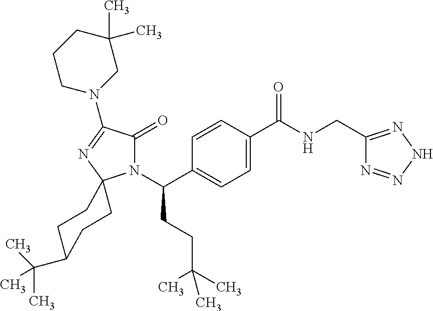 [SMILES: CC(C)(C)CC[C@H](c1ccc(C(=O)NCc2nn[nH]n2)cc1)N1C(=O)C(N2CCCC(C)(C)C2)=NC12CCC(C(C)(C)C)CC2] | 5 | 17.69 (20) | 617 |
| A-21 | 9.12 | 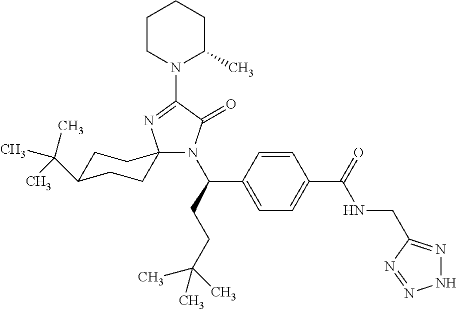 [SMILES: C[C@H]1CCCCN1C1=NC2(CCC(C(C)(C)C)CC2)N([C@H](CCC(C)(C)C)c2ccc(C(=O)NCc3nn[nH]n3)cc2)C1=O] | 5 | 15.96 (20) | 605 |
| A-22 | 9.13 | 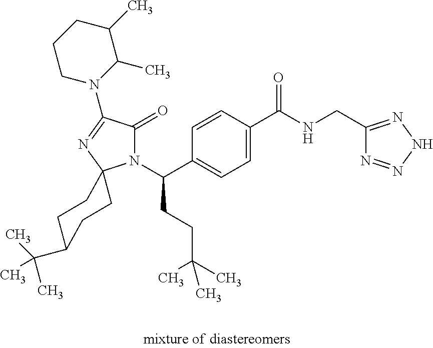 [SMILES: CC1CCCN(C2=NC3(CCC(C(C)(C)C)CC3)N([C@H](CCC(C)(C)C)c3ccc(C(=O)NCc4nn[nH]n4)cc3)C2=O)C1C] | 5 | 17.48 17.60 (20) | 619 |
| A-23 | 9.14 | 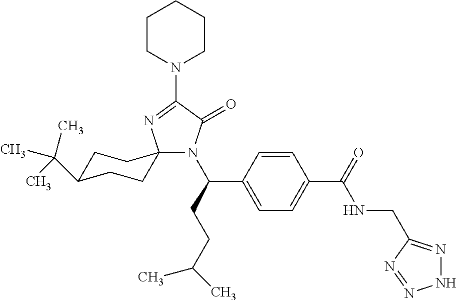 [SMILES: CC(C)CC[C@H](c1ccc(C(=O)NCc2nn[nH]n2)cc1)N1C(=O)C(N2CCCCC2)=NC12CCC(C(C)(C)C)CC2] | 5 | 15.16 (20) | 577 |
| A-24 | 9.15 | 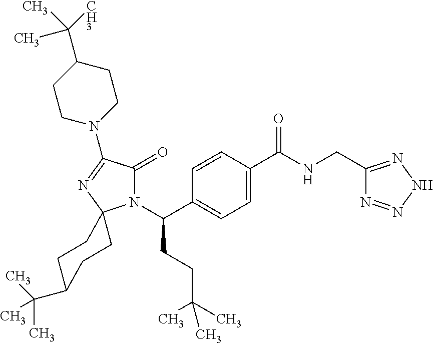 [SMILES: CC(C)(C)CC[C@H](c1ccc(C(=O)NCc2nn[nH]n2)cc1)N1C(=O)C(N2CCC(C(C)(C)C)CC2)=NC12CCC(C(C)(C)C)CC2] | 5 | 18.75 (20) | 647 |
| A-25 | 9.16 | 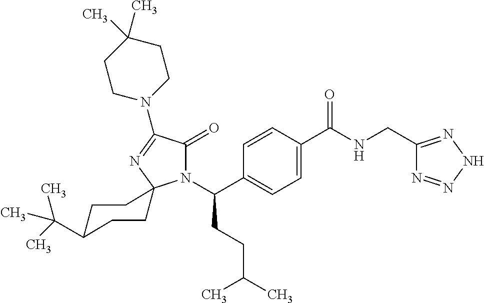 [SMILES: CC(C)CC[C@H](c1ccc(C(=O)NCc2nn[nH]n2)cc1)N1C(=O)C(N2CCC(C)(C)CC2)=NC12CCC(C(C)(C)C)CC2] | 5 | 13.62 (15) | 605 |
| A-26 | 9.17 | 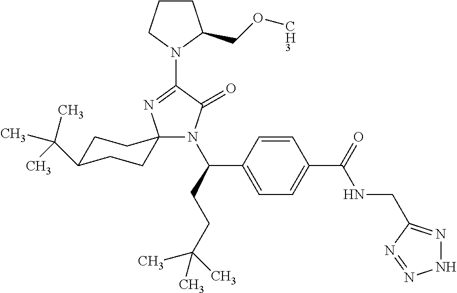 [SMILES: COC[C@@H]1CCCN1C1=NC2(CCC(C(C)(C)C)CC2)N([C@H](CCC(C)(C)C)c2ccc(C(=O)NCc3nn[nH]n3)cc2)C1=O] | 5 | 15.23 (20) | 621 |
| A-27 | 9.18 | 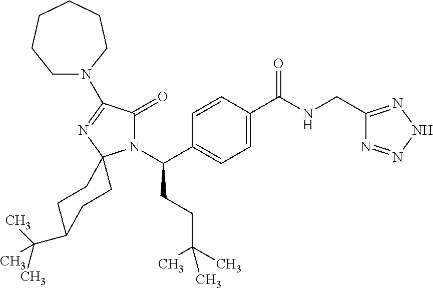 [SMILES: CC(C)(C)CC[C@H](c1ccc(C(=O)NCc2nn[nH]n2)cc1)N1C(=O)C(N2CCCCCC2)=NC12CCC(C(C)(C)C)CC2] | 5 | 16.83 (20) | 605 |
| A-28 | 9.19 | 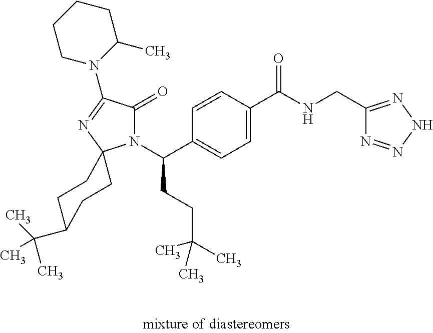 [SMILES: CC1CCCCN1C1=NC2(CCC(C(C)(C)C)CC2)N([C@H](CCC(C)(C)C)c2ccc(C(=O)NCc3nn[nH]n3)cc2)C1=O] | 5 | 17.02 17.11 (20) | 605 |
| A-29 | 9.20 | 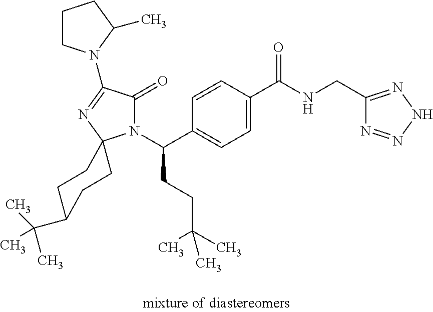 [SMILES: CC1CCCN1C1=NC2(CCC(C(C)(C)C)CC2)N([C@H](CCC(C)(C)C)c2ccc(C(=O)NCc3nn[nH]n3)cc2)C1=O] | 5 | 16.30 (20) | 591 |
| A-30 | 9.21 | 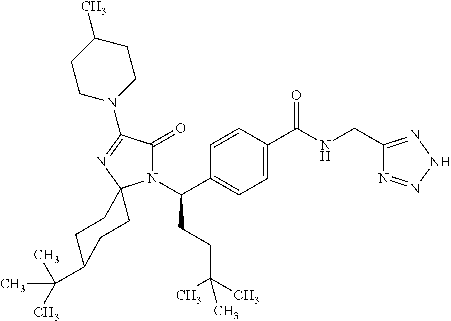 [SMILES: CC1CCN(C2=NC3(CCC(C(C)(C)C)CC3)N([C@H](CCC(C)(C)C)c3ccc(C(=O)NCc4nn[nH]n4)cc3)C2=O)CC1] | 5 | 16.24 (20) | 605 |
| A-32 | 9.22 | 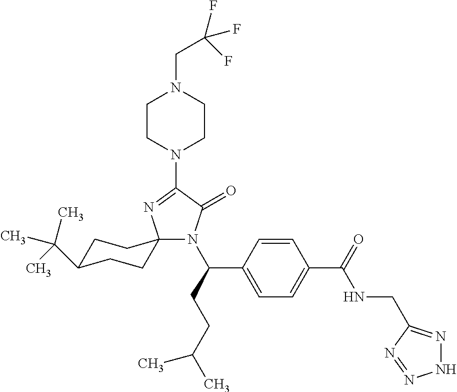 [SMILES: CC(C)CC[C@H](c1ccc(C(=O)NCc2nn[nH]n2)cc1)N1C(=O)C(N2CCN(CC(F)(F)F)CC2)=NC12CCC(C(C)(C)C)CC2] | 5 | 17.10 (20) | 660 |
| A-33 | 9.23 | 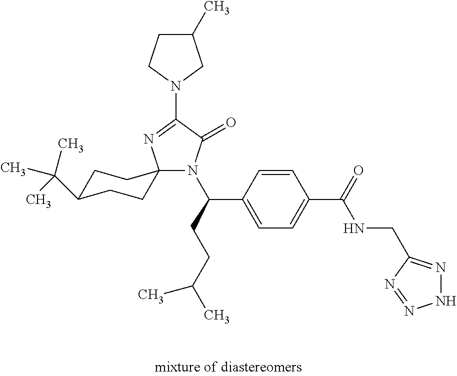 [SMILES: CC(C)CC[C@H](c1ccc(C(=O)NCc2nn[nH]n2)cc1)N1C(=O)C(N2CCC(C)C2)=NC12CCC(C(C)(C)C)CC2] | 5 | 14.87 (20) | 577 |
| A-34 | 9.24 | 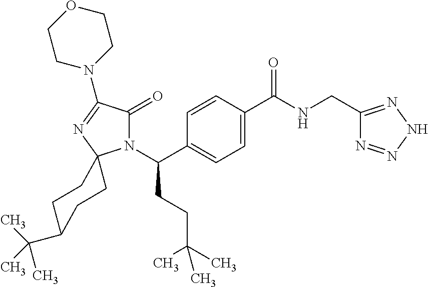 [SMILES: CC(C)(C)CC[C@H](c1ccc(C(=O)NCc2nn[nH]n2)cc1)N1C(=O)C(N2CCOCC2)=NC12CCC(C(C)(C)C)CC2] | 5 | 16.86 (20) | 593 |
| A-36 | 9.25 | 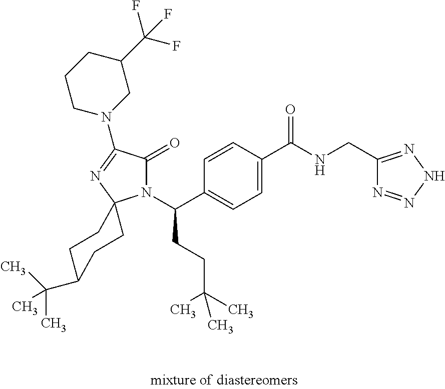 [SMILES: CC(C)(C)CC[C@H](c1ccc(C(=O)NCc2nn[nH]n2)cc1)N1C(=O)C(N2CCCC(C(F)(F)F)C2)=NC12CCC(C(C)(C)C)CC2] | 5 | 20.08 20.19 (20) | 657 |
| A-37 | 9.26 | 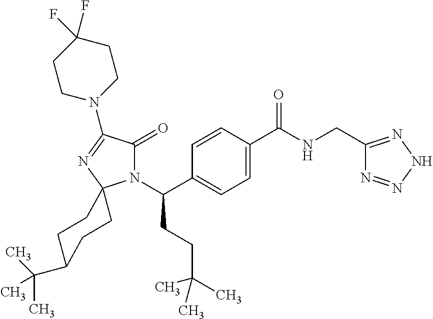 [SMILES: CC(C)(C)CC[C@H](c1ccc(C(=O)NCc2nn[nH]n2)cc1)N1C(=O)C(N2CCC(F)(F)CC2)=NC12CCC(C(C)(C)C)CC2] | 5 | 18.90 (20) | 627 |
| A-38 | 9.27 | 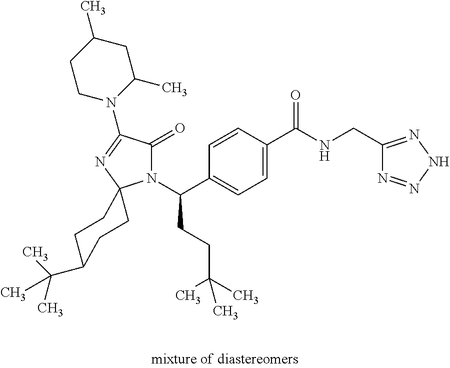 [SMILES: CC1CCN(C2=NC3(CCC(C(C)(C)C)CC3)N([C@H](CCC(C)(C)C)c3ccc(C(=O)NCc4nn[nH]n4)cc3)C2=O)C(C)C1] | 5 | 17.51 (20) | 619 |
| A-39 | 9.28 | 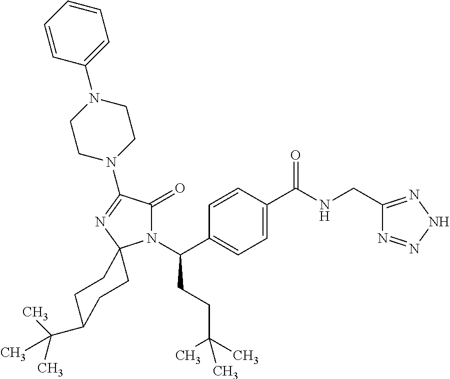 [SMILES: CC(C)(C)CC[C@H](c1ccc(C(=O)NCc2nn[nH]n2)cc1)N1C(=O)C(N2CCN(c3ccccc3)CC2)=NC12CCC(C(C)(C)C)CC2] | 5 | 19.12 (20) | 668 |
| A-89 | 9.29 | 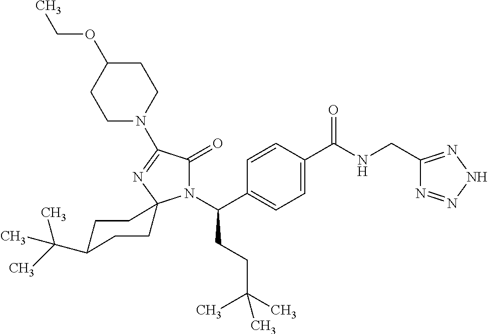 [SMILES: CCOC1CCN(C2=NC3(CCC(C(C)(C)C)CC3)N([C@H](CCC(C)(C)C)c3ccc(C(=O)NCc4nn[nH]n4)cc3)C2=O)CC1] | 5 | 15.84 (20) | 633 |
| A-41 | 9.30 | 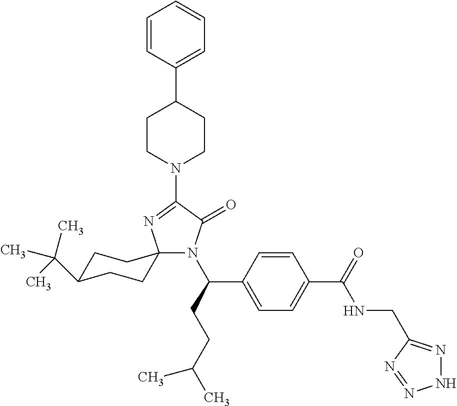 [SMILES: CC(C)CC[C@H](c1ccc(C(=O)NCc2nn[nH]n2)cc1)N1C(=O)C(N2CCC(c3ccccc3)CC2)=NC12CCC(C(C)(C)C)CC2] | 5 | 16.98 (20) | 653 |
| A-40 | 9.31 | 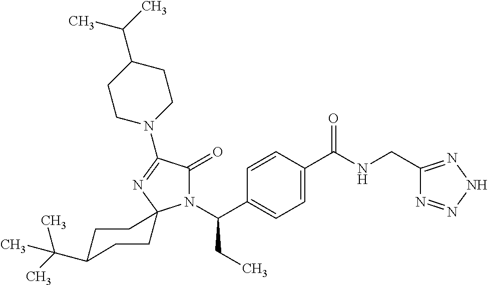 [SMILES: CC[C@H](c1ccc(C(=O)NCc2nn[nH]n2)cc1)N1C(=O)C(N2CCC(C(C)C)CC2)=NC12CCC(C(C)(C)C)CC2] | 5 | 17.97 (20) | 575 |
| A-42 | 9.32 | 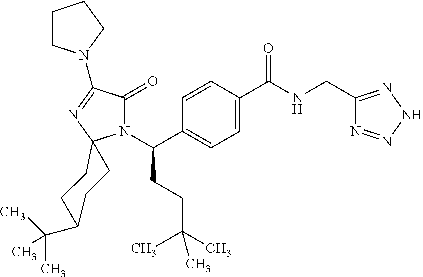 [SMILES: CC(C)(C)CC[C@H](c1ccc(C(=O)NCc2nn[nH]n2)cc1)N1C(=O)C(N2CCCC2)=NC12CCC(C(C)(C)C)CC2] | 5 | 15.87 (20) | 577 |
| A-43 | 9.33 | 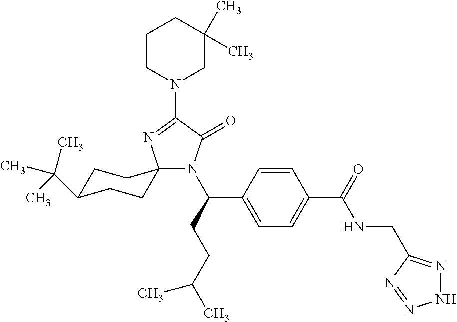 [SMILES: CC(C)CC[C@H](c1ccc(C(=O)NCc2nn[nH]n2)cc1)N1C(=O)C(N2CCCC(C)(C)C2)=NC12CCC(C(C)(C)C)CC2] | 5 | 16.30 (20) | 605 |
| A-44 | 9.34 | 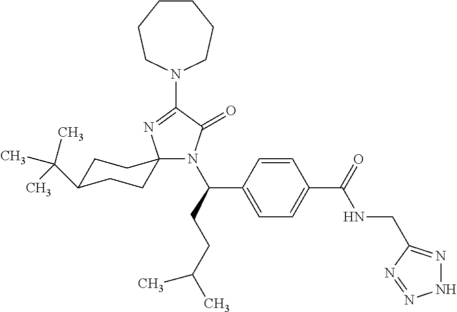 [SMILES: CC(C)CC[C@H](c1ccc(C(=O)NCc2nn[nH]n2)cc1)N1C(=O)C(N2CCCCCC2)=NC12CCC(C(C)(C)C)CC2] | 5 | 15.38 (20) | 591 |
| A-45 | 9.35 | 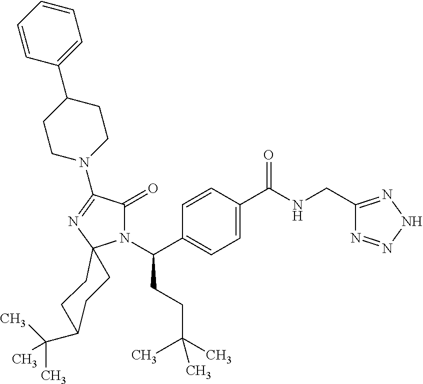 [SMILES: CC(C)(C)CC[C@H](c1ccc(C(=O)NCc2nn[nH]n2)cc1)N1C(=O)C(N2CCC(c3ccccc3)CC2)=NC12CCC(C(C)(C)C)CC2] | 5 | 18.38 (20) | 667 |
| A-46 | 9.36 | 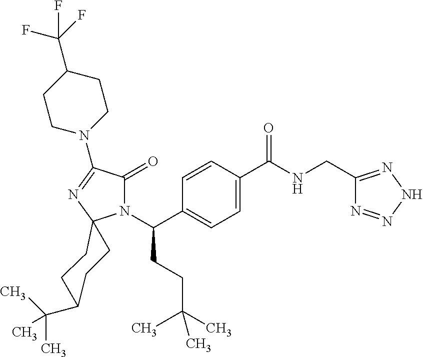 [SMILES: CC(C)(C)CC[C@H](c1ccc(C(=O)NCc2nn[nH]n2)cc1)N1C(=O)C(N2CCC(C(F)(F)F)CC2)=NC12CCC(C(C)(C)C)CC2] | 5 | 18.65 (20) | 659 |
| A-47 | 9.37 | 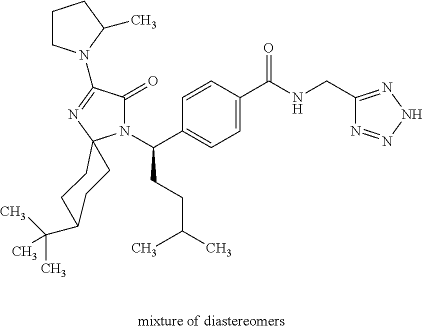 [SMILES: CC(C)CC[C@H](c1ccc(C(=O)NCc2nn[nH]n2)cc1)N1C(=O)C(N2CCCC2C)=NC12CCC(C(C)(C)C)CC2] | 5 | 15.82 (20) | 577 |
| B-7 | 9.38 | 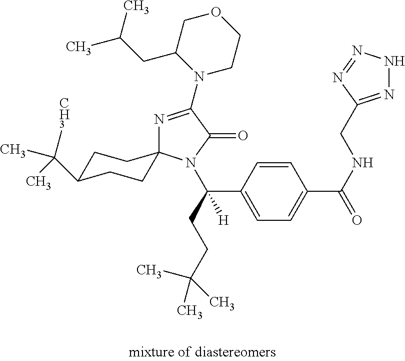 [SMILES: CC(C)CC1COCCN1C1=NC2(CCC(C(C)(C)C)CC2)N([C@H](CCC(C)(C)C)c2ccc(C(=O)NCc3nn[nH]n3)cc2)C1=O] | 1 | 2.25 | 649 |
| B-3 | 9.39 | 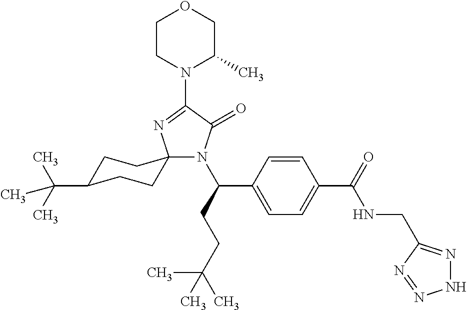 [SMILES: C[C@H]1COCCN1C1=NC2(CCC(C(C)(C)C)CC2)N([C@H](CCC(C)(C)C)c2ccc(C(=O)NCc3nn[nH]n3)cc2)C1=O] | 1 | 2.12 | 608 |
| B-5 | 9.40 | 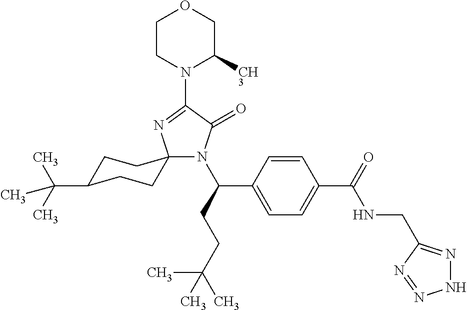 [SMILES: C[C@@H]1COCCN1C1=NC2(CCC(C(C)(C)C)CC2)N([C@H](CCC(C)(C)C)c2ccc(C(=O)NCc3nn[nH]n3)cc2)C1=O] | 1 | 2.13 | 608 |
| A-90 | 9.41 | 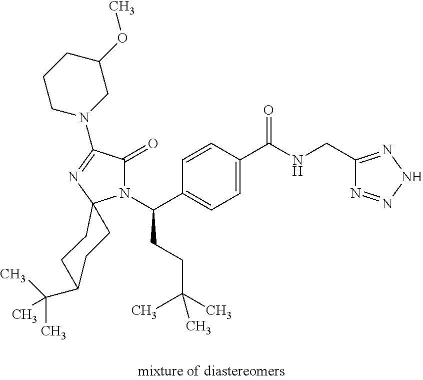 [SMILES: COC1CCCN(C2=NC3(CCC(C(C)(C)C)CC3)N([C@H](CCC(C)(C)C)c3ccc(C(=O)NCc4nn[nH]n4)cc3)C2=O)C1] | 5 | 16.68 (20) | 619 |
| A-49 | 9.42 | 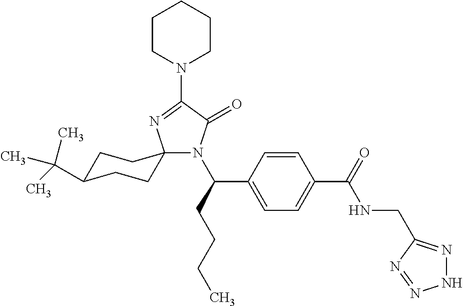 [SMILES: CCCC[C@H](c1ccc(C(=O)NCc2nn[nH]n2)cc1)N1C(=O)C(N2CCCCC2)=NC12CCC(C(C)(C)C)CC2] | 5 | 14.68 (20) | 563 |
| A-50 | 9.43 | 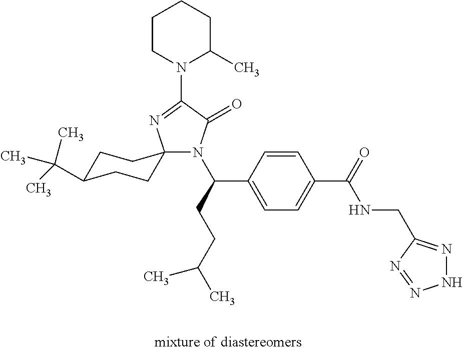 [SMILES: CC(C)CC[C@H](c1ccc(C(=O)NCc2nn[nH]n2)cc1)N1C(=O)C(N2CCCCC2C)=NC12CCC(C(C)(C)C)CC2] | 5 | 16.56 16.64 (20) | 591 |
| A-51 | 9.44 | 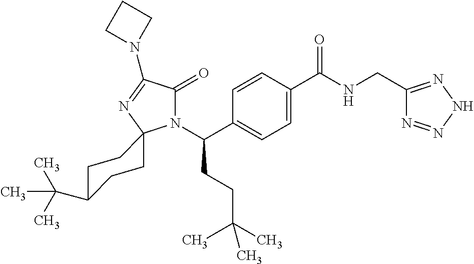 [SMILES: CC(C)(C)CC[C@H](c1ccc(C(=O)NCc2nn[nH]n2)cc1)N1C(=O)C(N2CCC2)=NC12CCC(C(C)(C)C)CC2] | 5 | 12.14 (15) | 563 |
| A-52 | 9.45 | 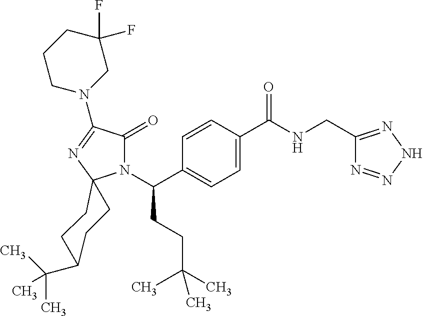 [SMILES: CC(C)(C)CC[C@H](c1ccc(C(=O)NCc2nn[nH]n2)cc1)N1C(=O)C(N2CCCC(F)(F)C2)=NC12CCC(C(C)(C)C)CC2] | 5 | 21.93 (20) | 627 |
| A-35 | 9.46 | 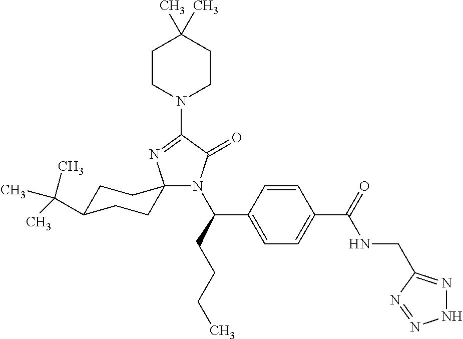 [SMILES: CCCC[C@H](c1ccc(C(=O)NCc2nn[nH]n2)cc1)N1C(=O)C(N2CCC(C)(C)CC2)=NC12CCC(C(C)(C)C)CC2] | 5 | 15.80 (20) | 591 |
| S-4 | 9.47 | 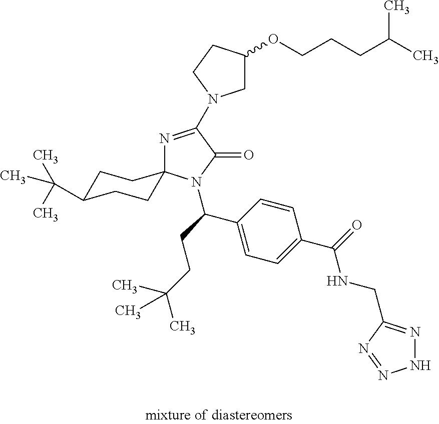 [SMILES: CC(C)CCCOC1CCN(C2=NC3(CCC(C(C)(C)C)CC3)N([C@H](CCC(C)(C)C)c3ccc(C(=O)NCc4nn[nH]n4)cc3)C2=O)C1] | 1 | 2.10 | 677.5 |
| B-6 | 9.48 | 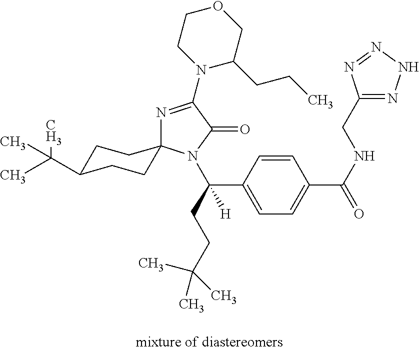 [SMILES: CCCC1COCCN1C1=NC2(CCC(C(C)(C)C)CC2)N([C@H](CCC(C)(C)C)c2ccc(C(=O)NCc3nn[nH]n3)cc2)C1=O] | 1 | 2.20 | 635 |
| B-4 | 9.49 | 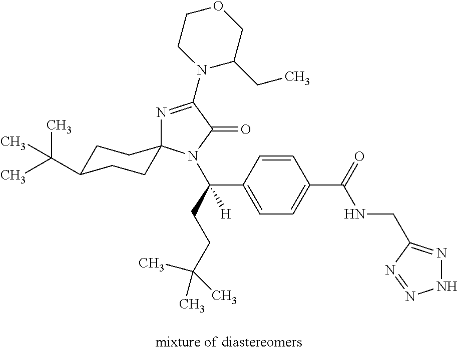 [SMILES: CCC1COCCN1C1=NC2(CCC(C(C)(C)C)CC2)N([C@H](CCC(C)(C)C)c2ccc(C(=O)NCc3nn[nH]n3)cc2)C1=O] | 1 | 2.15 | 621 |
| A-53 | 9.50 | 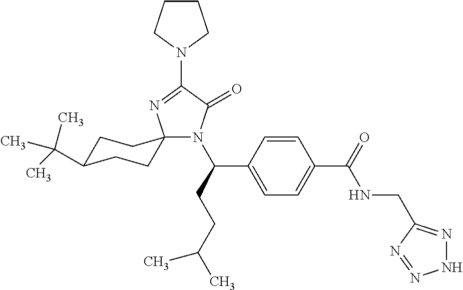 [SMILES: CC(C)CC[C@H](c1ccc(C(=O)NCc2nn[nH]n2)cc1)N1C(=O)C(N2CCCC2)=NC12CCC(C(C)(C)C)CC2] | 5 | 14.36 (20) | 563 |
| A-54 | 9.51 | 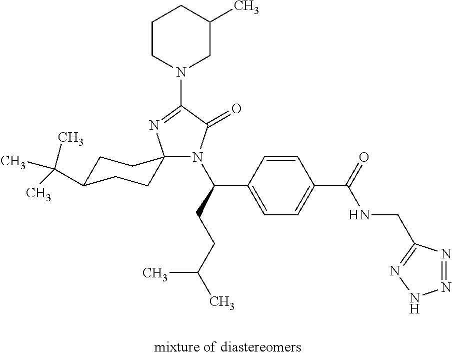 [SMILES: CC(C)CC[C@H](c1ccc(C(=O)NCc2nn[nH]n2)cc1)N1C(=O)C(N2CCCC(C)C2)=NC12CCC(C(C)(C)C)CC2] | 5 | 16.80 (20) | 591 |
| A-55 | 9.52 | 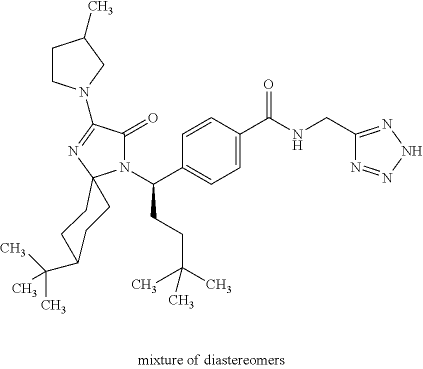 [SMILES: CC1CCN(C2=NC3(CCC(C(C)(C)C)CC3)N([C@H](CCC(C)(C)C)c3ccc(C(=O)NCc4nn[nH]n4)cc3)C2=O)C1] | 5 | 15.27 (20) | 589 |
| A-56 | 9.54 | 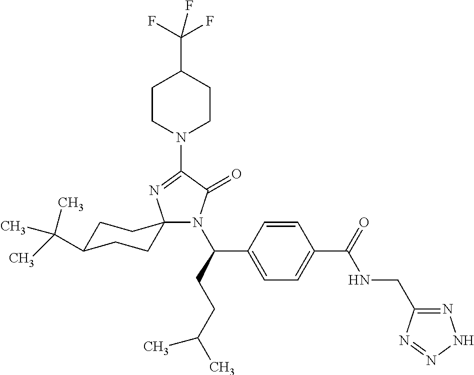 [SMILES: CC(C)CC[C@H](c1ccc(C(=O)NCc2nn[nH]n2)cc1)N1C(=O)C(N2CCC(C(F)(F)F)CC2)=NC12CCC(C(C)(C)C)CC2] | 5 | 18.16 (20) | 645 |
| A-57 | 9.55 | 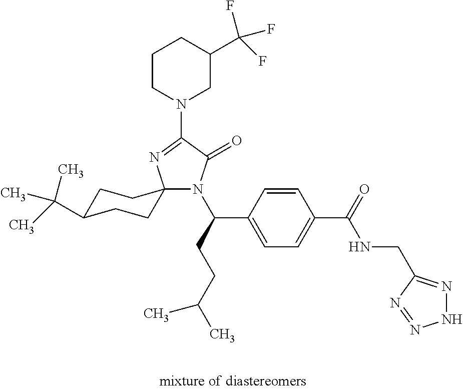 [SMILES: CC(C)CC[C@H](c1ccc(C(=O)NCc2nn[nH]n2)cc1)N1C(=O)C(N2CCCC(C(F)(F)F)C2)=NC12CCC(C(C)(C)C)CC2] | 5 | 19.66 (20) | 645 |
| A-58 | 9.56 | 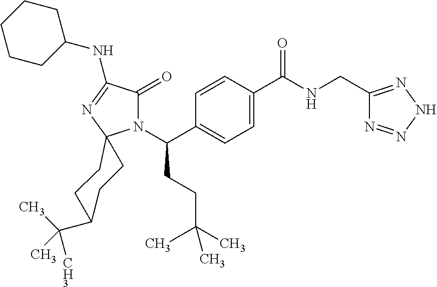 [SMILES: CC(C)(C)CC[C@H](c1ccc(C(=O)NCc2nn[nH]n2)cc1)N1C(=O)C(NC2CCCCC2)=NC12CCC(C(C)(C)C)CC2] | 5 | 16.80 (20) | 606 |
| A-60 | 9.57 | 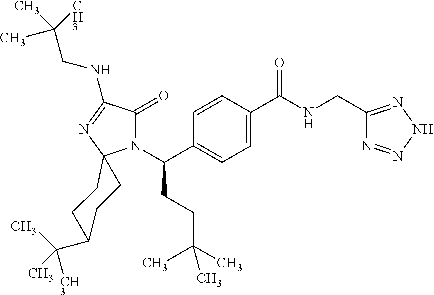 [SMILES: CC(C)(C)CC[C@H](c1ccc(C(=O)NCc2nn[nH]n2)cc1)N1C(=O)C(NCC(C)(C)C)=NC12CCC(C(C)(C)C)CC2] | 5 | 16.95 (20) | 593 |
| A-16 | 9.58 | 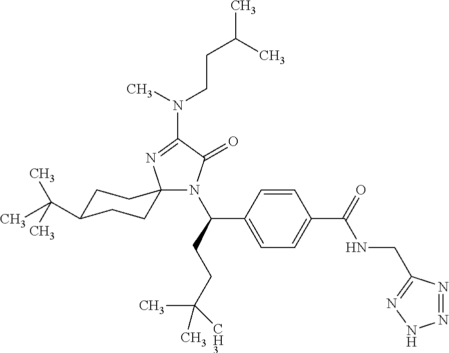 [SMILES: CC(C)CCN(C)C1=NC2(CCC(C(C)(C)C)CC2)N([C@H](CCC(C)(C)C)c2ccc(C(=O)NCc3nn[nH]n3)cc2)C1=O] | 2 | 3.74 | 607 |
| A-91 | 9.59 | 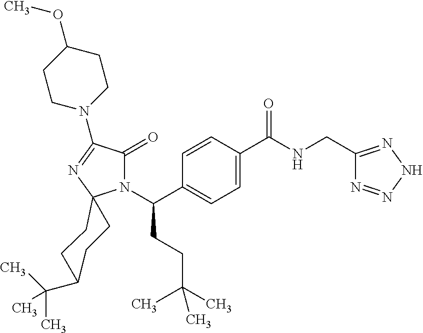 [SMILES: COC1CCN(C2=NC3(CCC(C(C)(C)C)CC3)N([C@H](CCC(C)(C)C)c3ccc(C(=O)NCc4nn[nH]n4)cc3)C2=O)CC1] | 1 | 2.14 | 621 |
| N-3 | 9.60 | 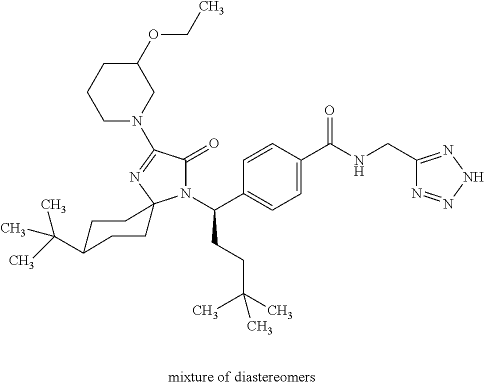 [SMILES: CCOC1CCCN(C2=NC3(CCC(C(C)(C)C)CC3)N([C@H](CCC(C)(C)C)c3ccc(C(=O)NCc4nn[nH]n4)cc3)C2=O)C1] | 5 | 14.40 (15) | 633 [M − H]⁻ |
| A-48 | 9.61 | 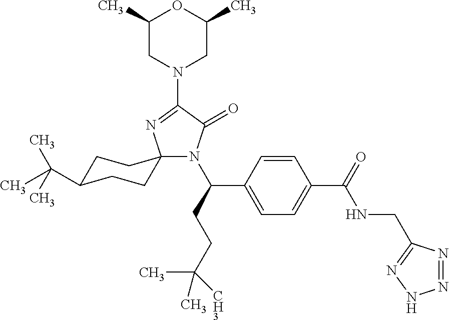 [SMILES: C[C@@H]1CN(C2=NC3(CCC(C(C)(C)C)CC3)N([C@H](CCC(C)(C)C)c3ccc(C(=O)NCc4nn[nH]n4)cc3)C2=O)C[C@H](C)O1] | 2 | 3.66 | 621 |
| A-62 | 9.62 | 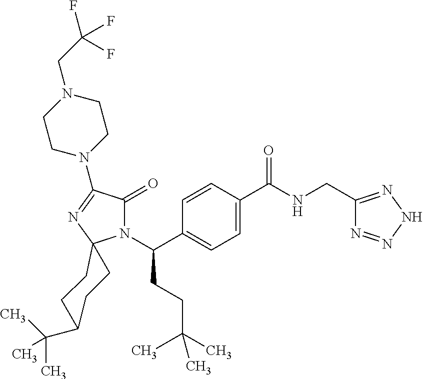 [SMILES: CC(C)(C)CC[C@H](c1ccc(C(=O)NCc2nn[nH]n2)cc1)N1C(=O)C(N2CCN(CC(F)(F)F)CC2)=NC12CCC(C(C)(C)C)CC2] | 1 | 2.19 | 674 |
| F-3 | 9.63 | 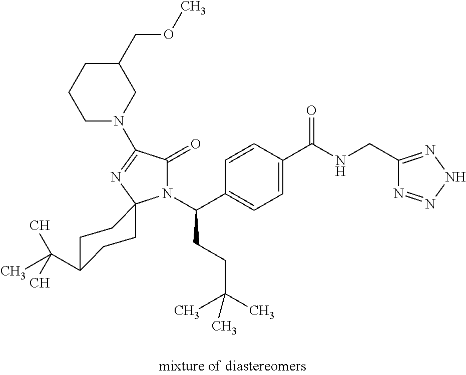 [SMILES: COCC1CCCN(C2=NC3(CCC(C(C)(C)C)CC3)N([C@H](CCC(C)(C)C)c3ccc(C(=O)NCc4nn[nH]n4)cc3)C2=O)C1] | 5 | 16.03 (20) | 633 [M − H]⁻ |
| A-31 | 9.64 | 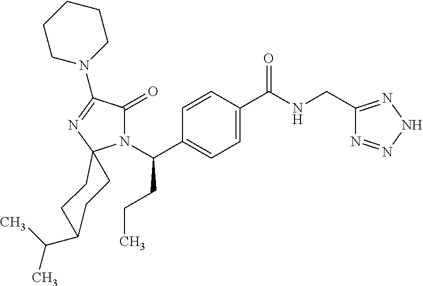 [SMILES: CCC[C@H](c1ccc(C(=O)NCc2nn[nH]n2)cc1)N1C(=O)C(N2CCCCC2)=NC12CCC(C(C)C)CC2] | 5 | 14.69 (20) | 535 |
| A-63 | 9.65 | 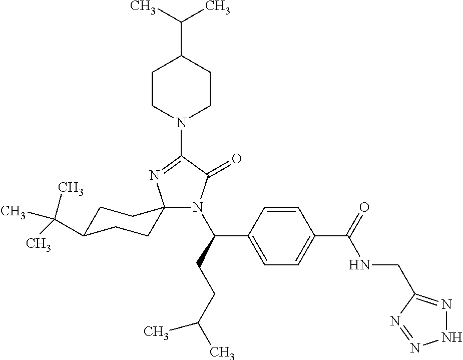 [SMILES: CC(C)CC[C@H](c1ccc(C(=O)NCc2nn[nH]n2)cc1)N1C(=O)C(N2CCC(C(C)C)CC2)=NC12CCC(C(C)(C)C)CC2] | 5 | 16.94 (20) | 619 |
| A-65 | 9.66 | 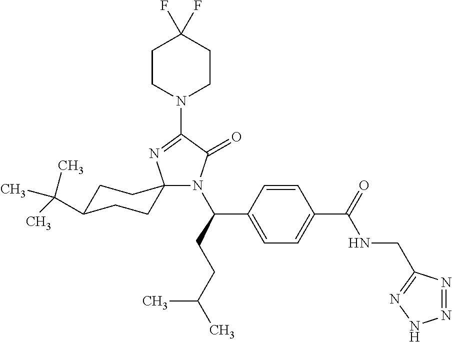 [SMILES: CC(C)CC[C@H](c1ccc(C(=O)NCc2nn[nH]n2)cc1)N1C(=O)C(N2CCC(F)(F)CC2)=NC12CCC(C(C)(C)C)CC2] | 5 | 19.38 (20) | 613 |
| A-66 | 9.67 | 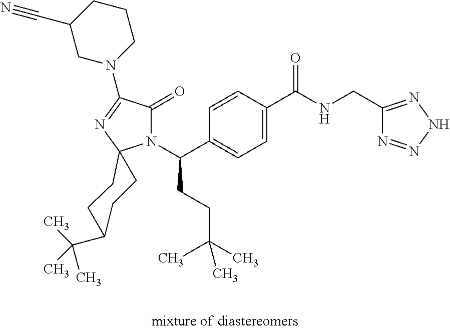 [SMILES: CC(C)(C)CC[C@H](c1ccc(C(=O)NCc2nn[nH]n2)cc1)N1C(=O)C(N2CCCC(C#N)C2)=NC12CCC(C(C)(C)C)CC2] | 5 | 18.54 18.13 (20) | 614 [M − H]⁻ |
| A-67 | 9.68 | 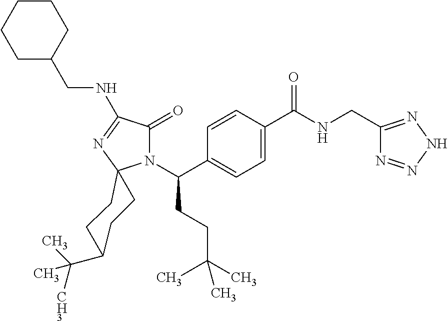 [SMILES: CC(C)(C)CC[C@H](c1ccc(C(=O)NCc2nn[nH]n2)cc1)N1C(=O)C(NCC2CCCCC2)=NC12CCC(C(C)(C)C)CC2] | 5 | 17.49 (20) | 619 |
| A-9 | 9.69 | 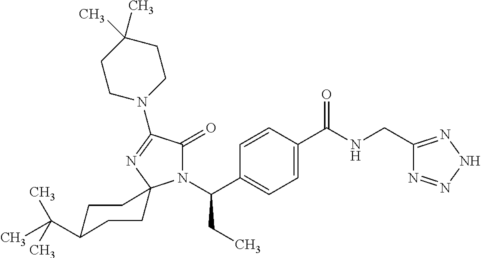 [SMILES: CC[C@H](c1ccc(C(=O)NCc2nn[nH]n2)cc1)N1C(=O)C(N2CCC(C)(C)CC2)=NC12CCC(C(C)(C)C)CC2] | 5 | 16.88 (20) | 561 [M − H]⁻ |
| A-68 | 9.70 | 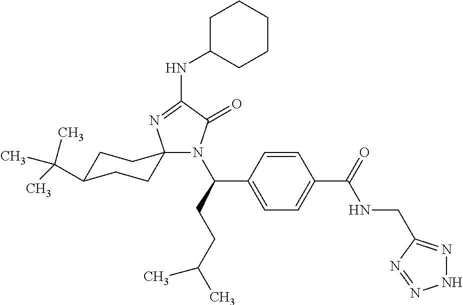 [SMILES: CC(C)CC[C@H](c1ccc(C(=O)NCc2nn[nH]n2)cc1)N1C(=O)C(NC2CCCCC2)=NC12CCC(C(C)(C)C)CC2] | 5 | 15.47 (20) | 591 |
| A-69 | 9.71 | 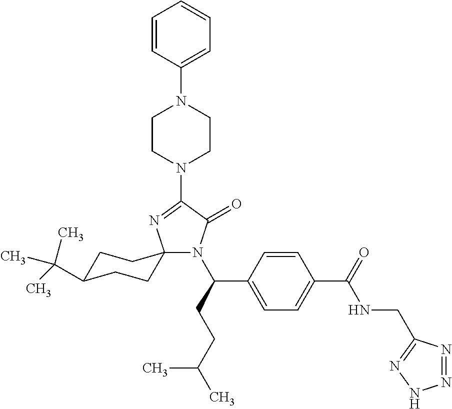 [SMILES: CC(C)CC[C@H](c1ccc(C(=O)NCc2nn[nH]n2)cc1)N1C(=O)C(N2CCN(c3ccccc3)CC2)=NC12CCC(C(C)(C)C)CC2] | 5 | 18.60 (20) | 654 |
| A-61 | 9.72 | 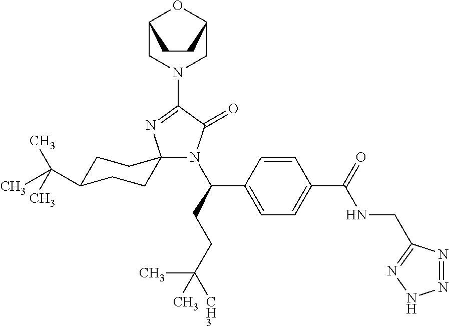 [SMILES: CC(C)(C)CC[C@H](c1ccc(C(=O)NCc2nn[nH]n2)cc1)N1C(=O)C(N2C[C@H]3CC[C@@H](C2)O3)=NC12CCC(C(C)(C)C)CC2] | 3 | 1.04 | 619 |
| A-92 | 9.73 | 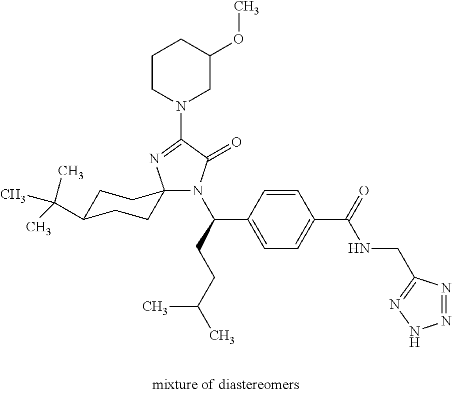 [SMILES: COC1CCCN(C2=NC3(CCC(C(C)(C)C)CC3)N([C@H](CCC(C)C)c3ccc(C(=O)NCc4nn[nH]n4)cc3)C2=O)C1] | 5 | 16.25 (20) | 607 |
| A-70 | 9.74 | 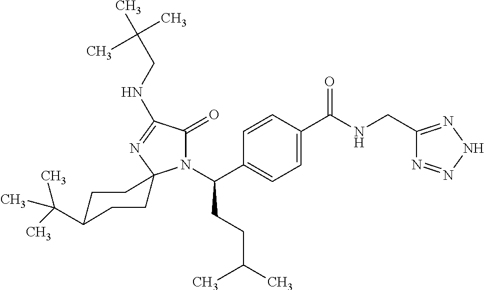 [SMILES: CC(C)CC[C@H](c1ccc(C(=O)NCc2nn[nH]n2)cc1)N1C(=O)C(NCC(C)(C)C)=NC12CCC(C(C)(C)C)CC2] | 5 | 13.03 (15) | 579 |
| A-71 | 9.75 | 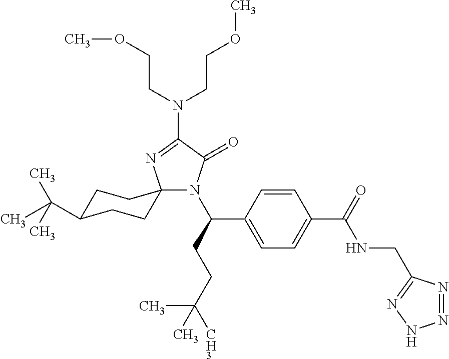 [SMILES: COCCN(CCOC)C1=NC2(CCC(C(C)(C)C)CC2)N([C@H](CCC(C)(C)C)c2ccc(C(=O)NCc3nn[nH]n3)cc2)C1=O] | 2 | 3.5 | 639 |
| A-72 | 9.76 | 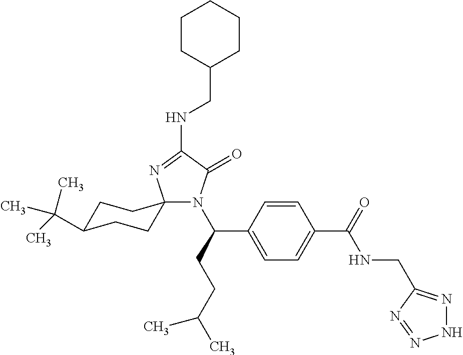 [SMILES: CC(C)CC[C@H](c1ccc(C(=O)NCc2nn[nH]n2)cc1)N1C(=O)C(NCC2CCCCC2)=NC12CCC(C(C)(C)C)CC2] | 5 | 17.07 (20) | 605 |
| A-93 | 9.77 | 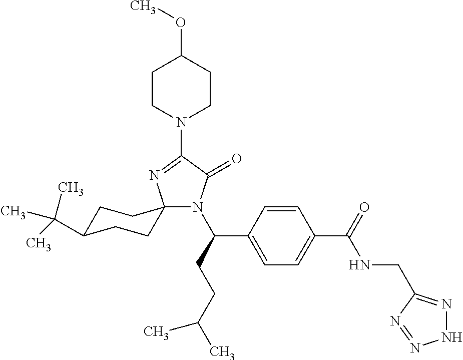 [SMILES: COC1CCN(C2=NC3(CCC(C(C)(C)C)CC3)N([C@H](CCC(C)C)c3ccc(C(=O)NCc4nn[nH]n4)cc3)C2=O)CC1] | 5 | 14.89 (20) | 607 |
| A-73 | 9.78 | 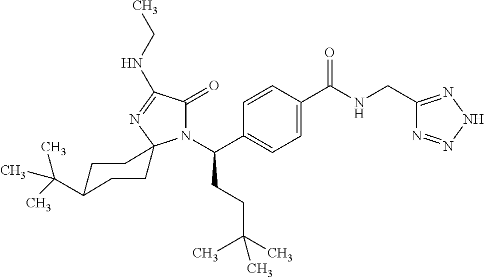 [SMILES: CCNC1=NC2(CCC(C(C)(C)C)CC2)N([C@H](CCC(C)(C)C)c2ccc(C(=O)NCc3nn[nH]n3)cc2)C1=O] | 5 | 12.07 (15) | 551 |
| A-74 | 9.79 | 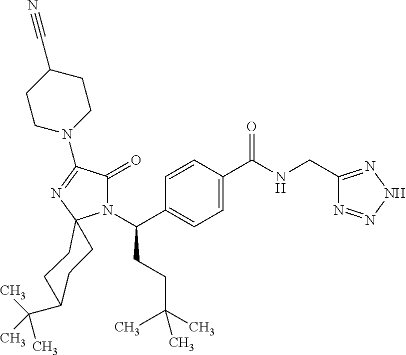 [SMILES: CC(C)(C)CC[C@H](c1ccc(C(=O)NCc2nn[nH]n2)cc1)N1C(=O)C(N2CCC(C#N)CC2)=NC12CCC(C(C)(C)C)CC2] | 5 | 17.10 (20) | 614 [M − ]⁻ |
| A-59 | 9.81 | 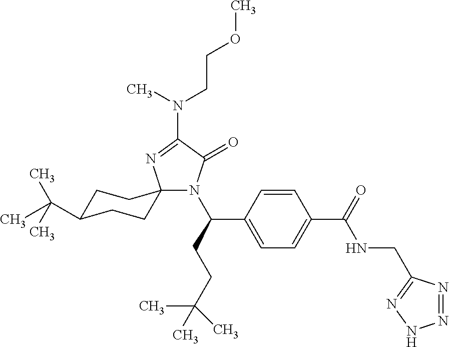 [SMILES: COCCN(C)C1=NC2(CCC(C(C)(C)C)CC2)N([C@H](CCC(C)(C)C)c2ccc(C(=O)NCc3nn[nH]n3)cc2)C1=O] | 2 | 3.39 | 595 |
| A-75 | 9.82 | 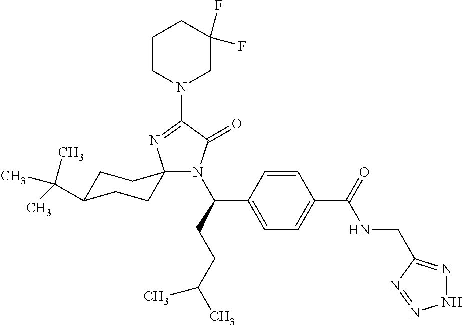 [SMILES: CC(C)CC[C@H](c1ccc(C(=O)NCc2nn[nH]n2)cc1)N1C(=O)C(N2CCCC(F)(F)C2)=NC12CCC(C(C)(C)C)CC2] | 5 | 20.63 (20) | 613 |
| A-19 | 9.83 | 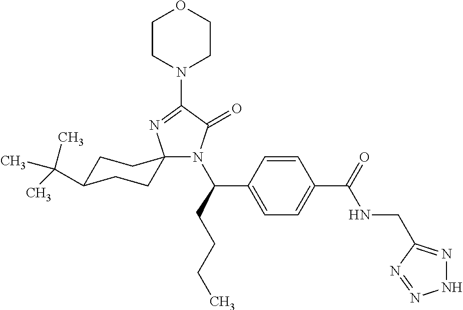 [SMILES: CCCC[C@H](c1ccc(C(=O)NCc2nn[nH]n2)cc1)N1C(=O)C(N2CCOCC2)=NC12CCC(C(C)(C)C)CC2] | 5 | 14.85 (20) | 565 |
| A-76 | 9.84 | 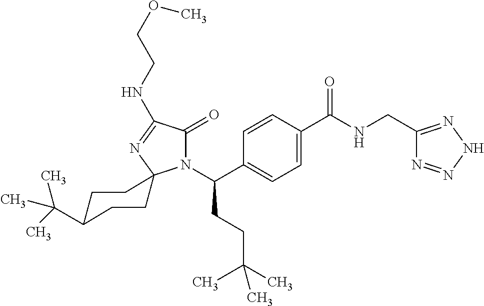 [SMILES: COCCNC1=NC2(CCC(C(C)(C)C)CC2)N([C@H](CCC(C)(C)C)c2ccc(C(=O)NCc3nn[nH]n3)cc2)C1=O] | 5 | 12.13 (15) | 581 |
| A-77 | 9.86 | 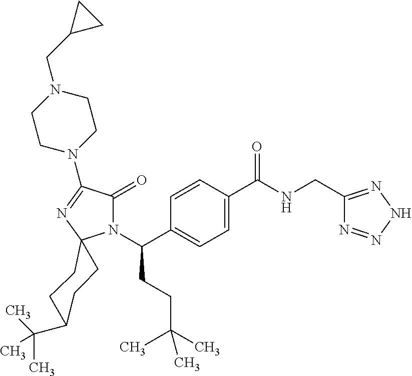 [SMILES: CC(C)(C)CC[C@H](c1ccc(C(=O)NCc2nn[nH]n2)cc1)N1C(=O)C(N2CCN(CC3CC3)CC2)=NC12CCC(C(C)(C)C)CC2] | 5 | 16.25 (20) | 646 |
| A-78 | 9.87 | 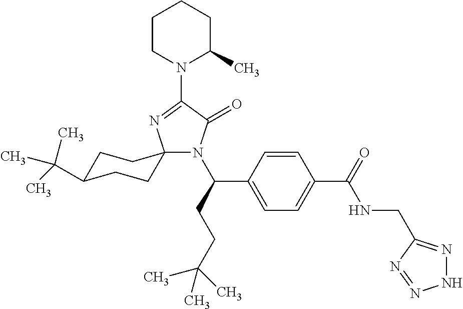 [SMILES: C[C@@H]1CCCCN1C1=NC2(CCC(C(C)(C)C)CC2)N([C@H](CCC(C)(C)C)c2ccc(C(=O)NCc3nn[nH]n3)cc2)C1=O] | 5 | 16.13 (20) | 605 |
| A-80 | 9.88 | 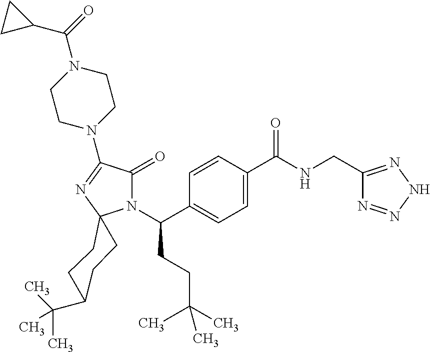 [SMILES: CC(C)(C)CC[C@H](c1ccc(C(=O)NCc2nn[nH]n2)cc1)N1C(=O)C(N2CCN(C(=O)C3CC3)CC2)=NC12CCC(C(C)(C)C)CC2] | 5 | 16.83 (20) | 660 |
| A-81 | 9.89 | 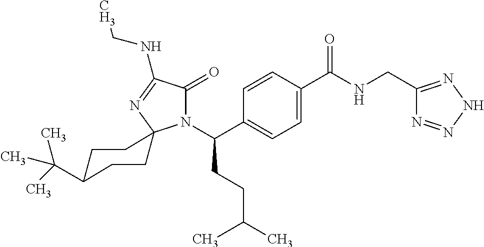 [SMILES: CCNC1=NC2(CCC(C(C)(C)C)CC2)N([C@H](CCC(C)C)c2ccc(C(=O)NCc3nn[nH]n3)cc2)C1=O] | 5 | 11.87 915) | 537 |
| A-82 | 9.90 | 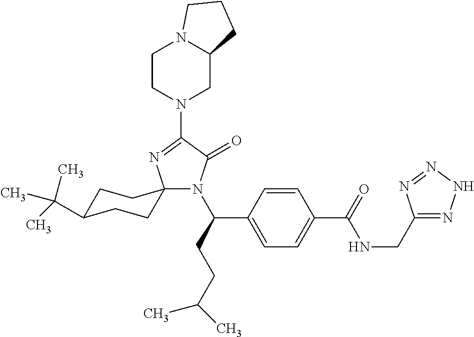 [SMILES: CC(C)CC[C@H](c1ccc(C(=O)NCc2nn[nH]n2)cc1)N1C(=O)C(N2CCN3CCC[C@H]3C2)=NC12CCC(C(C)(C)C)CC2] | 5 | 15.18 (20) | 618 |
| A-64 | 9.91 | 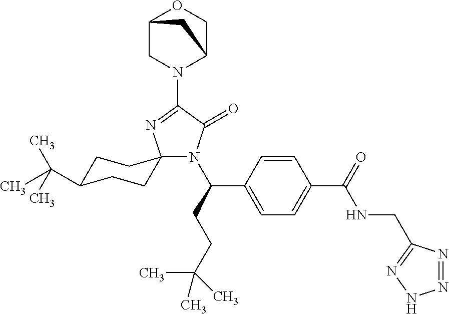 [SMILES: CC(C)(C)CC[C@H](c1ccc(C(=O)NCc2nn[nH]n2)cc1)N1C(=O)C(N2C[C@H]3C[C@@H]2CO3)=NC12CCC(C(C)(C)C)CC2] | 3 | 1.0 | 605 |
| A-79 | 9.92 | 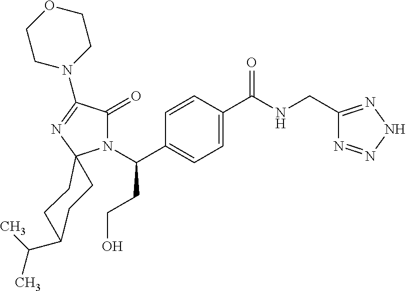 [SMILES: CC(C)C1CCC2(CC1)N=C(N1CCOCC1)C(=O)N2[C@H](CCO)c1ccc(C(=O)NCc2nn[nH]n2)cc1] | 5 | 16.61 (20) | 537 |
| A-83 | 9.93 | 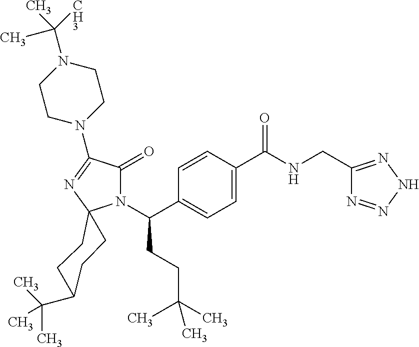 [SMILES: CC(C)(C)CC[C@H](c1ccc(C(=O)NCc2nn[nH]n2)cc1)N1C(=O)C(N2CCN(C(C)(C)C)CC2)=NC12CCC(C(C)(C)C)CC2] | 5 | 15.90 (20) | 648 |
| A-84 | 9.94 | 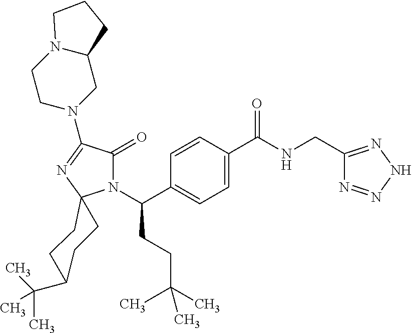 [SMILES: CC(C)(C)CC[C@H](c1ccc(C(=O)NCc2nn[nH]n2)cc1)N1C(=O)C(N2CCN3CCC[C@H]3C2)=NC12CCC(C(C)(C)C)CC2] | 5 | 15.62 (20) | 632 |
| A-85 | 9.95 | 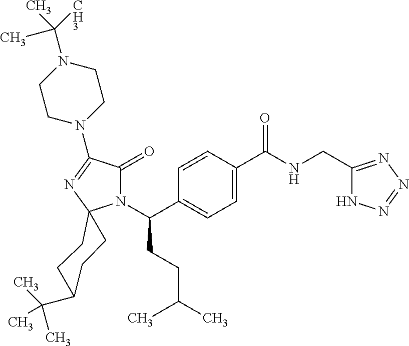 [SMILES: CC(C)CC[C@H](c1ccc(C(=O)NCc2nnn[nH]2)cc1)N1C(=O)C(N2CCN(C(C)(C)C)CC2)=NC12CCC(C(C)(C)C)CC2] | 5 | 15.49 (20) | 634 |
| A-86 | 9.96 | 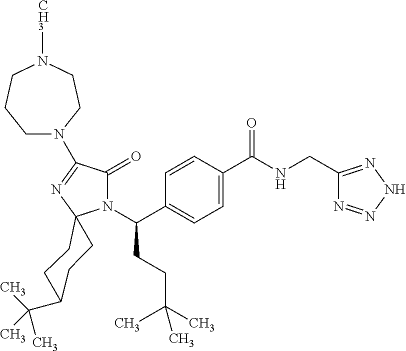 [SMILES: CN1CCCN(C2=NC3(CCC(C(C)(C)C)CC3)N([C@H](CCC(C)(C)C)c3ccc(C(=O)NCc4nn[nH]n4)cc3)C2=O)CC1] | 5 | 14.00 (20) | 620 |
| A-87 | 9.97 | 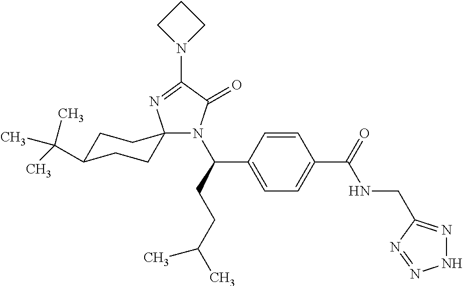 [SMILES: CC(C)CC[C@H](c1ccc(C(=O)NCc2nn[nH]n2)cc1)N1C(=O)C(N2CCC2)=NC12CCC(C(C)(C)C)CC2] | 5 | 13.93 (20) | 549 |
| A-88 | 9.98 | 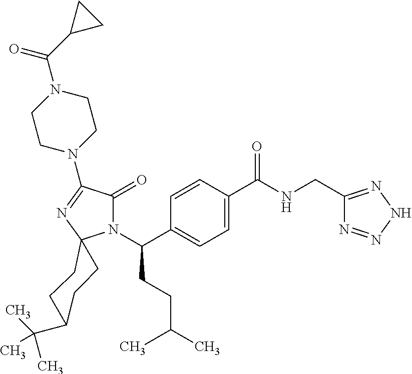 [SMILES: CC(C)CC[C@H](c1ccc(C(=O)NCc2nn[nH]n2)cc1)N1C(=O)C(N2CCN(C(=O)C3CC3)CC2)=NC12CCC(C(C)(C)C)CC2] | 5 | 16.36 (20) | 644 [M −H]⁻ |
| C-6 | 9.99 | 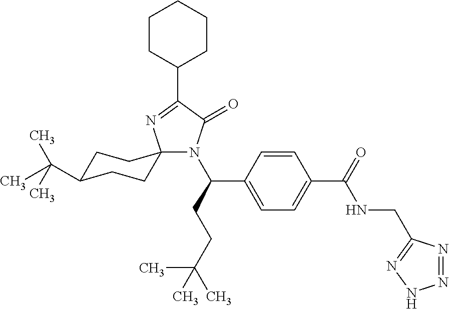 [SMILES: CC(C)(C)CC[C@H](c1ccc(C(=O)NCc2nn[nH]n2)cc1)N1C(=O)C(C2CCCCC2)=NC12CCC(C(C)(C)C)CC2] | 1 | 2.52 | 590 |
| I-10 | 9.100 | 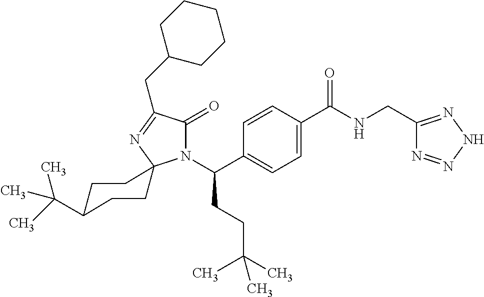 [SMILES: CC(C)(C)CC[C@H](c1ccc(C(=O)NCc2nn[nH]n2)cc1)N1C(=O)C(CC2CCCCC2)=NC12CCC(C(C)(C)C)CC2] | 6 | 22.77 | 604 |
| I-8 | 9.101 | 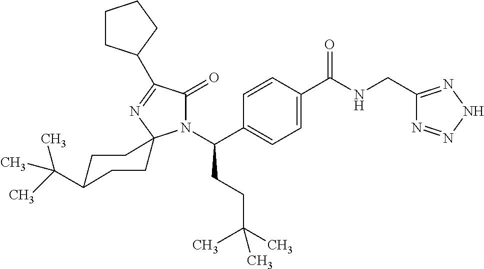 [SMILES: CC(C)(C)CC[C@H](c1ccc(C(=O)NCc2nn[nH]n2)cc1)N1C(=O)C(C2CCCC2)=NC12CCC(C(C)(C)C)CC2] | 6 | 20.89 | 576 |
| I-16 | 9.102 | 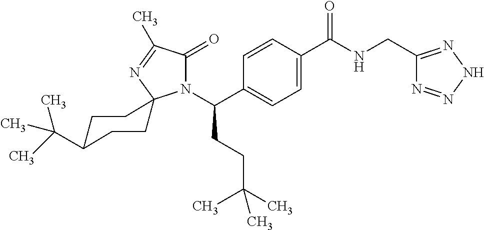 [SMILES: CC1=NC2(CCC(C(C)(C)C)CC2)N([C@H](CCC(C)(C)C)c2ccc(C(=O)NCc3nn[nH]n3)cc2)C1=O] | 5 | 18.62 (15) | 522 |
| I-13 | 9.103 | 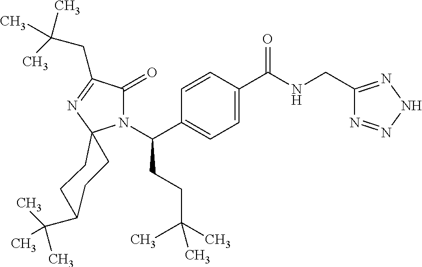 [SMILES: CC(C)(C)CC[C@H](c1ccc(C(=O)NCc2nn[nH]n2)cc1)N1C(=O)C(CC(C)(C)C)=NC12CCC(C(C)(C)C)CC2] | 6 | 20.01 | 578 |
| I-12 | 9.104 | 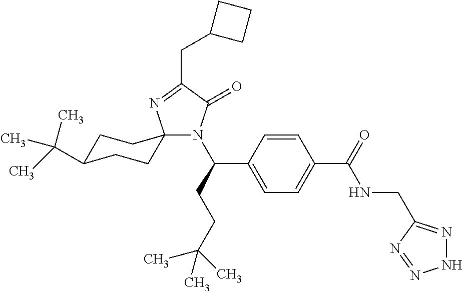 [SMILES: CC(C)(C)CC[C@H](c1ccc(C(=O)NCc2nn[nH]n2)cc1)N1C(=O)C(CC2CCC2)=NC12CCC(C(C)(C)C)CC2] | 6 | 19.49 | 576 |
| I-9 | 9.105 | 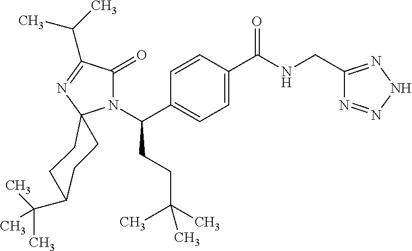 [SMILES: CC(C)C1=NC2(CCC(C(C)(C)C)CC2)N([C@H](CCC(C)(C)C)c2ccc(C(=O)NCc3nn[nH]n3)cc2)C1=O] | 6 | 18.27 | 550 |
| I-19 | 9.106 | 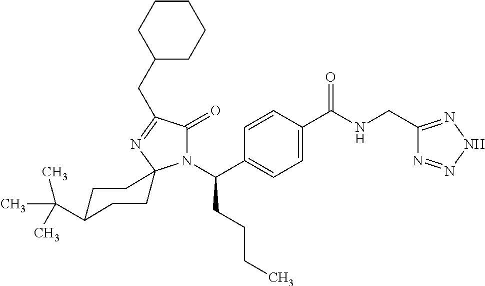 [SMILES: CCCC[C@H](c1ccc(C(=O)NCc2nn[nH]n2)cc1)N1C(=O)C(CC2CCCCC2)=NC12CCC(C(C)(C)C)CC2] | 6 | 19.31 | 574 [M − H]⁻ |
| I-17 | 9.107 | 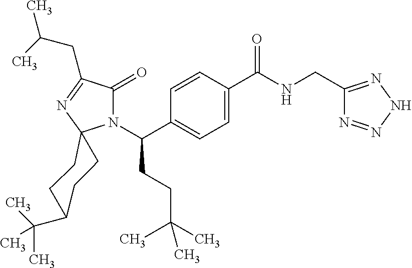 [SMILES: CC(C)CC1=NC2(CCC(C(C)(C)C)CC2)N([C@H](CCC(C)(C)C)c2ccc(C(=O)NCc3nn[nH]n3)cc2)C1=O] | 6 | 18.10 | 564 |
| I-6 | 9.108 | 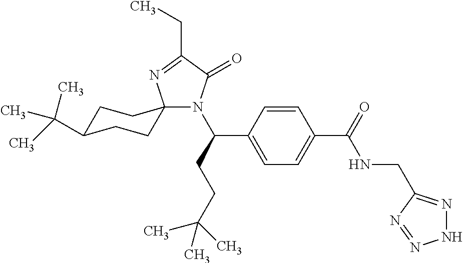 [SMILES: CCC1=NC2(CCC(C(C)(C)C)CC2)N([C@H](CCC(C)(C)C)c2ccc(C(=O)NCc3nn[nH]n3)cc2)C1=O] | 6 | 15.56 | 536 |
| I-14 | 9.109 | 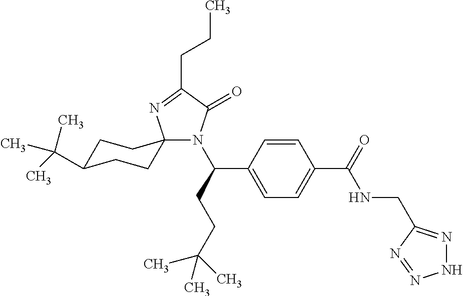 [SMILES: CCCC1=NC2(CCC(C(C)(C)C)CC2)N([C@H](CCC(C)(C)C)c2ccc(C(=O)NCc3nn[nH]n3)cc2)C1=O] | 6 | 16.93 | 550 |
| C-5 | 9.110 | 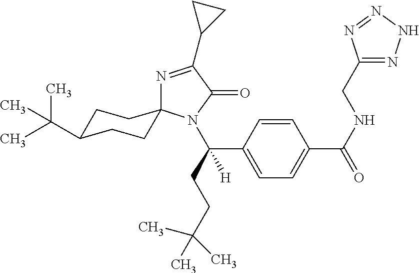 [SMILES: CC(C)(C)CC[C@H](c1ccc(C(=O)NCc2nn[nH]n2)cc1)N1C(=O)C(C2CC2)=NC12CCC(C(C)(C)C)CC2] | 1 | 2.35 | 548 |
| I-15 | 9.111 | 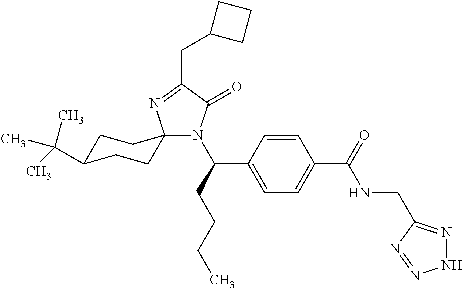 [SMILES: CCCC[C@H](c1ccc(C(=O)NCc2nn[nH]n2)cc1)N1C(=O)C(CC2CCC2)=NC12CCC(C(C)(C)C)CC2] | 6 | 16.89 | 548 |
| I-21 | 9.112 | 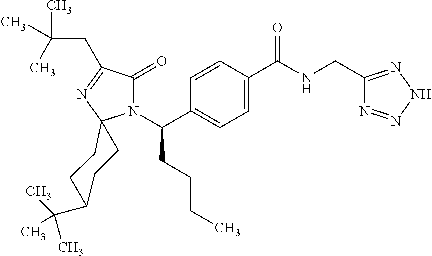 [SMILES: CCCC[C@H](c1ccc(C(=O)NCc2nn[nH]n2)cc1)N1C(=O)C(CC(C)(C)C)=NC12CCC(C(C)(C)C)CC2] | 6 | 17.20 | 550 |
| I-5 | 9.113 | 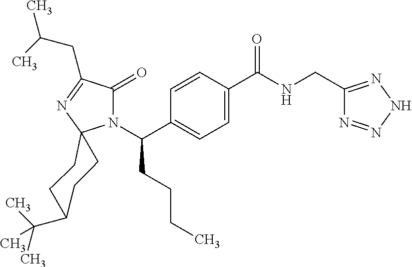 [SMILES: CCCC[C@H](c1ccc(C(=O)NCc2nn[nH]n2)cc1)N1C(=O)C(CC(C)C)=NC12CCC(C(C)(C)C)CC2] | 6 | 16.03 | 536 |
| I-18 | 9.114 | 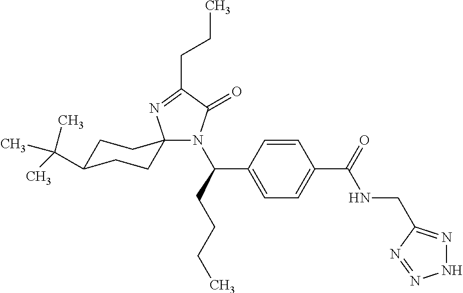 [SMILES: CCCC[C@H](c1ccc(C(=O)NCc2nn[nH]n2)cc1)N1C(=O)C(CCC)=NC12CCC(C(C)(C)C)CC2] | 6 | 14.88 | 522 |
| I-20 | 9.115 | 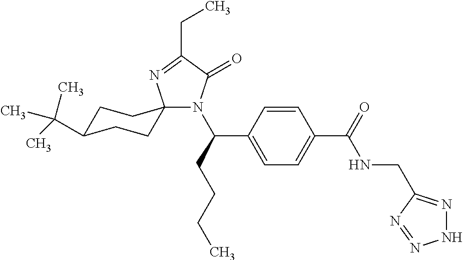 [SMILES: CCCC[C@H](c1ccc(C(=O)NCc2nn[nH]n2)cc1)N1C(=O)C(CC)=NC12CCC(C(C)(C)C)CC2] | 6 | 13.68 | 508 |
| I-15 | 9.116 | 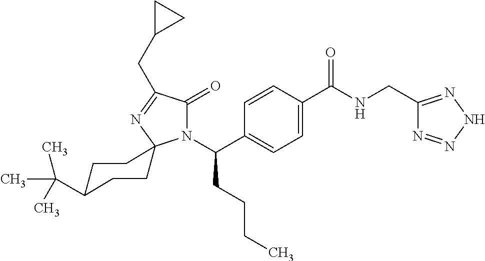 [SMILES: CCCC[C@H](c1ccc(C(=O)NCc2nn[nH]n2)cc1)N1C(=O)C(CC2CC2)=NC12CCC(C(C)(C)C)CC2] | 5 | 19.63 (15) | 534 |
| I-22 | 9.117 | 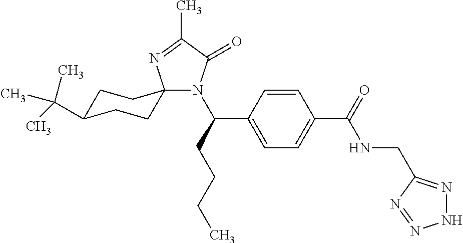 [SMILES: CCCC[C@H](c1ccc(C(=O)NCc2nn[nH]n2)cc1)N1C(=O)C(C)=NC12CCC(C(C)(C)C)CC2] | 6 | 11.47 | 494 |
| O-1 | 9.118 | 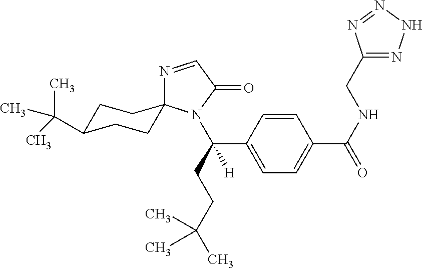 [SMILES: CC(C)(C)CC[C@H](c1ccc(C(=O)NCc2nn[nH]n2)cc1)N1C(=O)C=NC12CCC(C(C)(C)C)CC2] | 1 | 2.24 | 508 |
| B-9 | 9.119 | 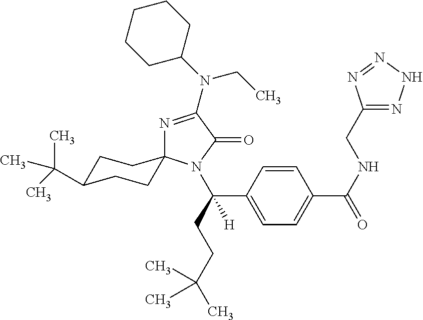 [SMILES: CCN(C1=NC2(CCC(C(C)(C)C)CC2)N([C@H](CCC(C)(C)C)c2ccc(C(=O)NCc3nn[nH]n3)cc2)C1=O)C1CCCCC1] | 1 | 2.27 | 634 |
| B-10 | 9.120 | 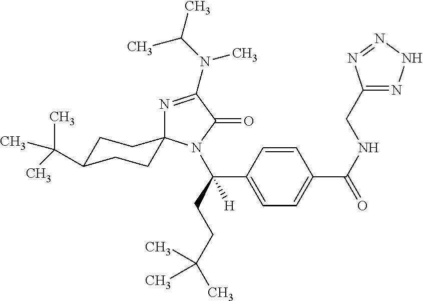 [SMILES: CC(C)N(C)C1=NC2(CCC(C(C)(C)C)CC2)N([C@H](CCC(C)(C)C)c2ccc(C(=O)NCc3nn[nH]n3)cc2)C1=O] | 1 | 2.16 | 580 |
| A-95 | 9.121 | 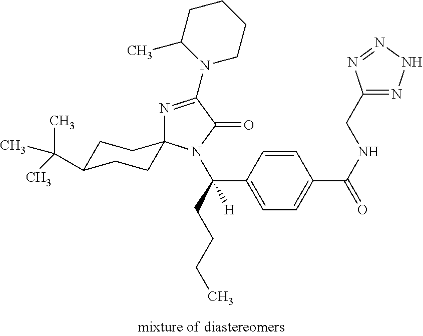 [SMILES: CCCC[C@H](c1ccc(C(=O)NCc2nn[nH]n2)cc1)N1C(=O)C(N2CCCCC2C)=NC12CCC(C(C)(C)C)CC2] | 5 | 15.18 (20) | 577 |
| A-97 | 9.122 | 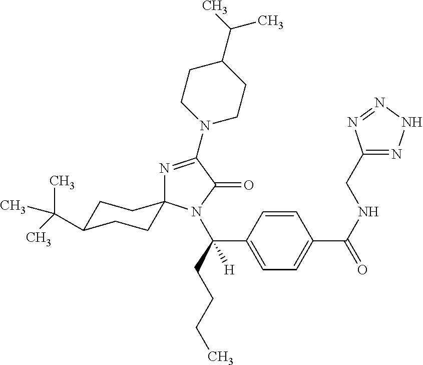 [SMILES: CCCC[C@H](c1ccc(C(=O)NCc2nn[nH]n2)cc1)N1C(=O)C(N2CCC(C(C)C)CC2)=NC12CCC(C(C)(C)C)CC2] | 5 | 16.49 (20) | 603 [M − H]⁻ |
| A-96 | 9.123 | 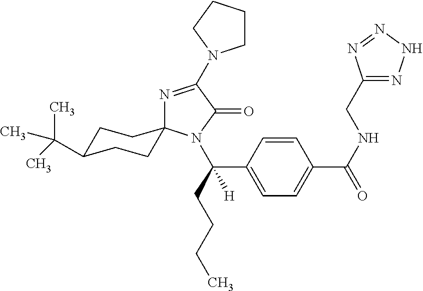 [SMILES: CCCC[C@H](c1ccc(C(=O)NCc2nn[nH]n2)cc1)N1C(=O)C(N2CCCC2)=NC12CCC(C(C)(C)C)CC2] | 5 | 13.91 (20) | 549 |
| A-94 | 9.124 | 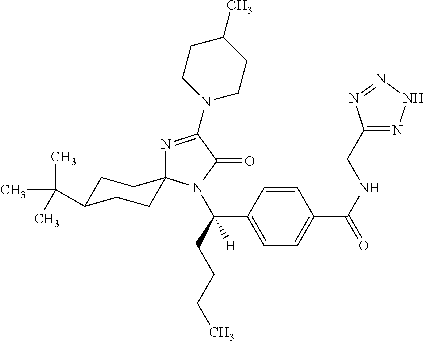 [SMILES: CCCC[C@H](c1ccc(C(=O)NCc2nn[nH]n2)cc1)N1C(=O)C(N2CCC(C)CC2)=NC12CCC(C(C)(C)C)CC2] | 5 | 15.40 (20) | 577 |
| T-3 | 9.125 | 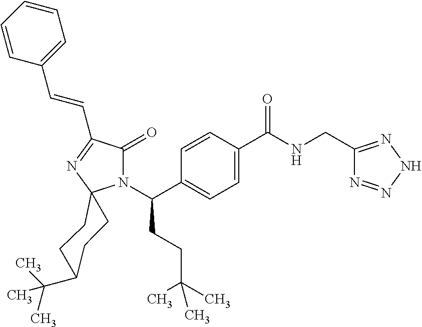 [SMILES: CC(C)(C)CC[C@H](c1ccc(C(=O)NCc2nn[nH]n2)cc1)N1C(=O)C(/C=C/c2ccccc2)=NC12CCC(C(C)(C)C)CC2] | 3 | 1.35 | 610 |
| T-4 | 9.126 | 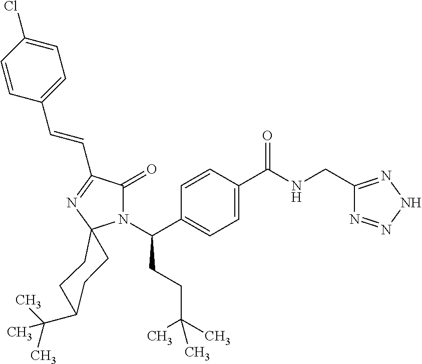 [SMILES: CC(C)(C)CC[C@H](c1ccc(C(=O)NCc2nn[nH]n2)cc1)N1C(=O)C(/C=C/c2ccc(Cl)cc2)=NC12CCC(C(C)(C)C)CC2] | 3 | 1.39 | 644 |
| T-5 | 9.127 | 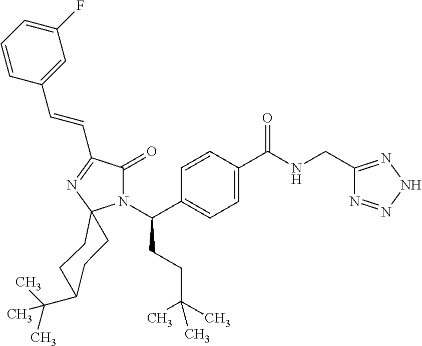 [SMILES: CC(C)(C)CC[C@H](c1ccc(C(=O)NCc2nn[nH]n2)cc1)N1C(=O)C(/C=C/c2cccc(F)c2)=NC12CCC(C(C)(C)C)CC2] | 3 | 1.35 | 628 |
| T-6 | 9.128 | 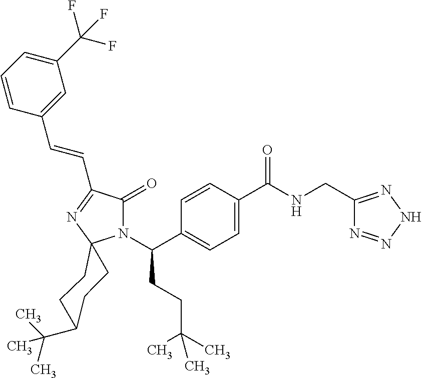 [SMILES: CC(C)(C)CC[C@H](c1ccc(C(=O)NCc2nn[nH]n2)cc1)N1C(=O)C(/C=C/c2cccc(C(F)(F)F)c2)=NC12CCC(C(C)(C)C)CC2] | 3 | 1.39 | 678 |
| B-11 | 9.129 | 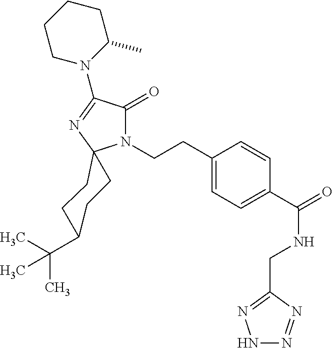 [SMILES: C[C@H]1CCCCN1C1=NC2(CCC(C(C)(C)C)CC2)N(CCc2ccc(C(=O)NCc3nn[nH]n3)cc2)C1=O] | 1 | | |
| B-12 | 9.130 | 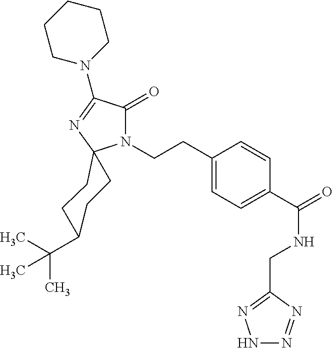 [SMILES: CC(C)(C)C1CCC2(CC1)N=C(N1CCCCC1)C(=O)N2CCc1ccc(C(=O)NCc2nn[nH]n2)cc1] | 1 | | |

TABLE 10
Using the requisite starting material, and a method similar to that outlined in
either Scheme E or Scheme K, the following compounds were prepared:

| | | | LCMS | | |
|---|---|---|---|---|---|
| Benzoic | | | | Ret | |
| Acid | Ex. | Structure | LC | (min) | [M + H]⁺ |
| A-8 | 10.1 | 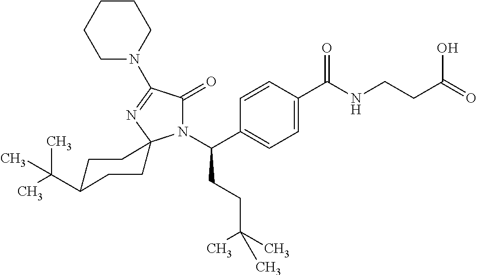 [SMILES: CC(C)(C)CC[C@H](c1ccc(C(=O)NCCC(=O)O)cc1)N1C(=O)C(N2CCCCC2)=NC12CCC(C(C)(C)C)CC2] | 5 | 15.57 (20) | 581 |
| A-9 | 10.2 | 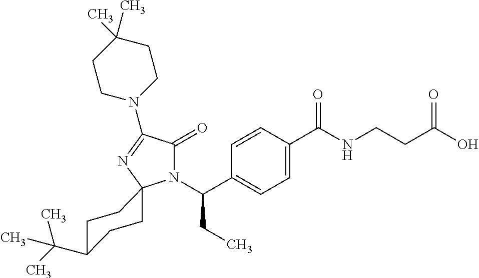 [SMILES: CC[C@H](c1ccc(C(=O)NCCC(=O)O)cc1)N1C(=O)C(N2CCC(C)(C)CC2)=NC12CCC(C(C)(C)C)CC2] | 5 | 16.78 (20) | 553 |
| B-4 | 10.3 | 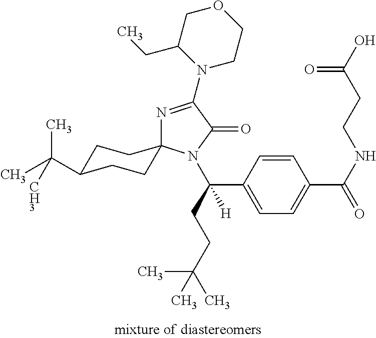 [SMILES: CCC1COCCN1C1=NC2(CCC(C(C)(C)C)CC2)N([C@H](CCC(C)(C)C)c2ccc(C(=O)NCCC(=O)O)cc2)C1=O] | 1 | 2.18 | 612 |
| A-15 | 10.4 | 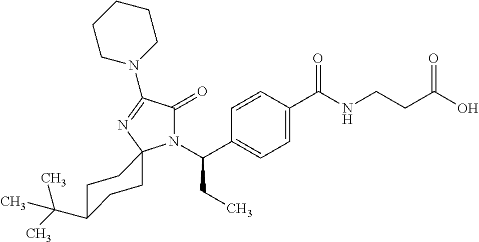 [SMILES: CC[C@H](c1ccc(C(=O)NCCC(=O)O)cc1)N1C(=O)C(N2CCCCC2)=NC12CCC(C(C)(C)C)CC2] | 5 | 15.54 (20) | 525 |
| B-6 | 10.5 | 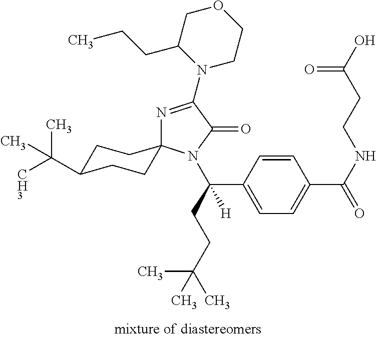 [SMILES: CCCC1COCCN1C1=NC2(CCC(C(C)(C)C)CC2)N([C@H](CCC(C)(C)C)c2ccc(C(=O)NCCC(=O)O)cc2)C1=O] | 1 | 2.19 | 625 |
| A-16 | 10.6 | 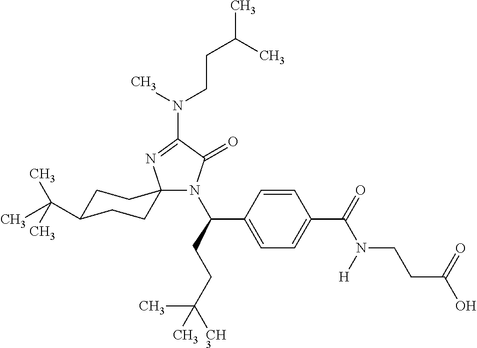 [SMILES: CC(C)CCN(C)C1=NC2(CCC(C(C)(C)C)CC2)N([C@H](CCC(C)(C)C)c2ccc(C(=O)NCCC(=O)O)cc2)C1=O] | 3 | 1.13 | 597 |
| B-5 | 10.7 | 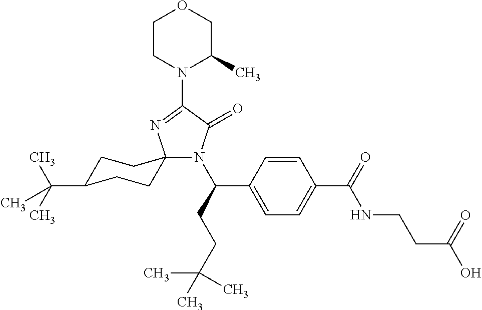 [SMILES: C[C@@H]1COCCN1C1=NC2(CCC(C(C)(C)C)CC2)N([C@H](CCC(C)(C)C)c2ccc(C(=O)NCCC(=O)O)cc2)C1=O] | 1 | 2.14 | 598 |
| A-19 | 10.8 | 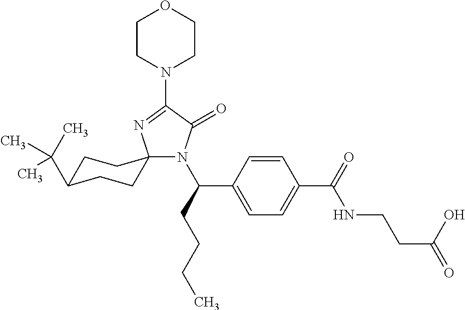 [SMILES: CCCC[C@H](c1ccc(C(=O)NCCC(=O)O)cc1)N1C(=O)C(N2CCOCC2)=NC12CCC(C(C)(C)C)CC2] | 5 | 14.78 (20) | 555 |
| B-7 | 10.9 | 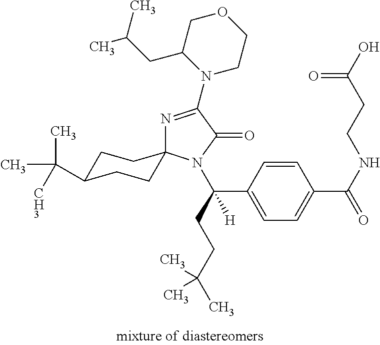 [SMILES: CC(C)CC1COCCN1C1=NC2(CCC(C(C)(C)C)CC2)N([C@H](CCC(C)(C)C)c2ccc(C(=O)NCCC(=O)O)cc2)C1=O] | 1 | 2.26 | 639 |
| B-3 | 10.10 | 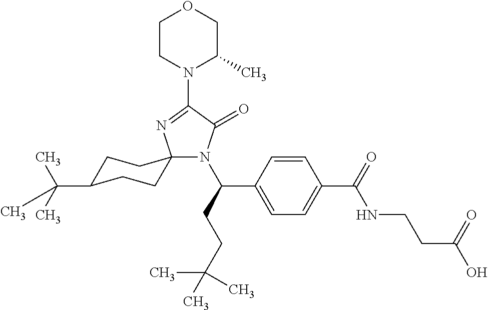 [SMILES: C[C@H]1COCCN1C1=NC2(CCC(C(C)(C)C)CC2)N([C@H](CCC(C)(C)C)c2ccc(C(=O)NCCC(=O)O)cc2)C1=O] | 1 | 2.13 | 598 |
| A-31 | 10.11 | 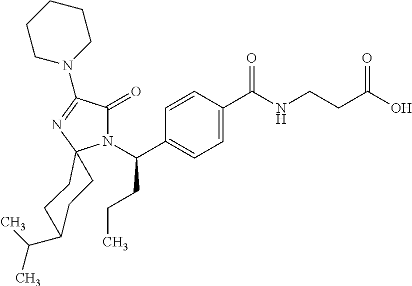 [SMILES: CCC[C@H](c1ccc(C(=O)NCCC(=O)O)cc1)N1C(=O)C(N2CCCCC2)=NC12CCC(C(C)C)CC2] | 5 | 14.71 (20) | 525 |
| A-35 | 10.12 | 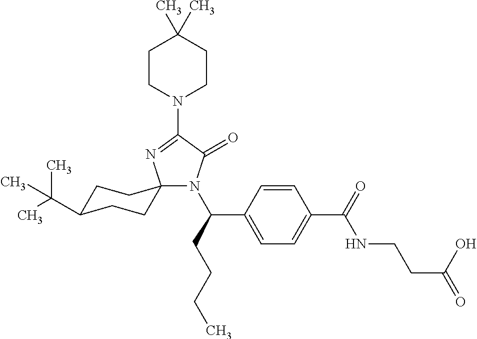 [SMILES: CCCC[C@H](c1ccc(C(=O)NCCC(=O)O)cc1)N1C(=O)C(N2CCC(C)(C)CC2)=NC12CCC(C(C)(C)C)CC2] | 5 | 15.79 (20) | 581 |
| B-8 | 10.13 | 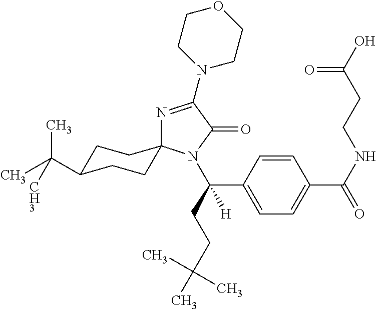 [SMILES: CC(C)(C)CC[C@H](c1ccc(C(=O)NCCC(=O)O)cc1)N1C(=O)C(N2CCOCC2)=NC12CCC(C(C)(C)C)CC2] | 1 | 1.86 | 583 |
| A-40 | 10.14 | 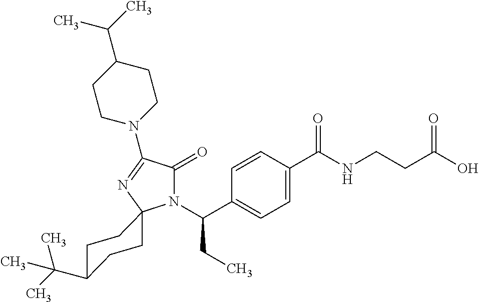 [SMILES: CC[C@H](c1ccc(C(=O)NCCC(=O)O)cc1)N1C(=O)C(N2CCC(C(C)C)CC2)=NC12CCC(C(C)(C)C)CC2] | 5 | 17.60 (20) | 567 |
| A-48 | 10.15 | 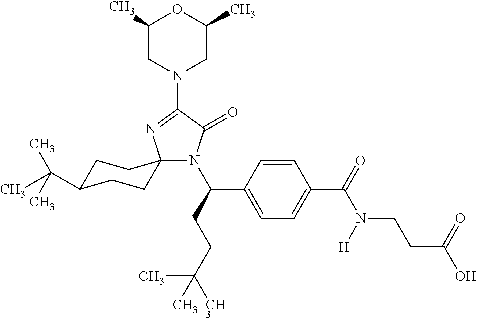 [SMILES: C[C@@H]1CN(C2=NC3(CCC(C(C)(C)C)CC3)N([C@H](CCC(C)(C)C)c3ccc(C(=O)NCCC(=O)O)cc3)C2=O)C[C@H](C)O1] | 3 | 1.09 | 611 |
| A-59 | 10.16 | 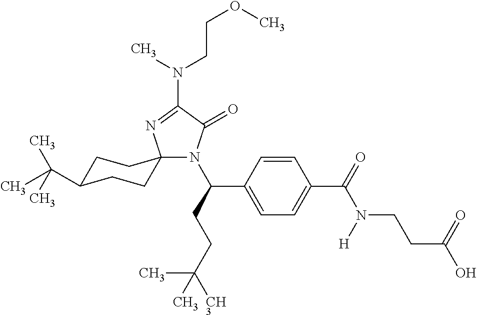 [SMILES: COCCN(C)C1=NC2(CCC(C(C)(C)C)CC2)N([C@H](CCC(C)(C)C)c2ccc(C(=O)NCCC(=O)O)cc2)C1=O] | 3 | 1.05 | 585 |
| A-61 | 10.17 | 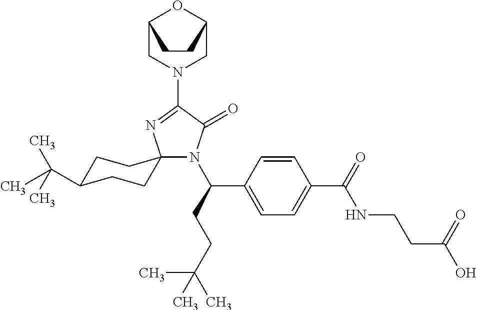 [SMILES: CC(C)(C)CC[C@H](c1ccc(C(=O)NCCC(=O)O)cc1)N1C(=O)C(N2C[C@H]3CC[C@@H](C2)O3)=NC12CCC(C(C)(C)C)CC2] | 3 | 1.05 | 609 |
| A-64 | 10.18 | 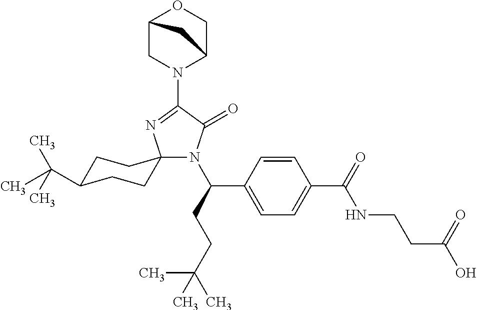 [SMILES: CC(C)(C)CC[C@H](c1ccc(C(=O)NCCC(=O)O)cc1)N1C(=O)C(N2C[C@H]3C[C@@H]2CO3)=NC12CCC(C(C)(C)C)CC2] | 3 | 1.02 | 595 |
| A-49 | 10.19 | 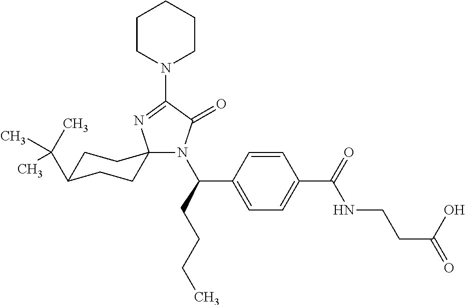 [SMILES: CCCC[C@H](c1ccc(C(=O)NCCC(=O)O)cc1)N1C(=O)C(N2CCCCC2)=NC12CCC(C(C)(C)C)CC2] | 5 | 14.80 (20) | 553 |
| A-71 | 10.20 | 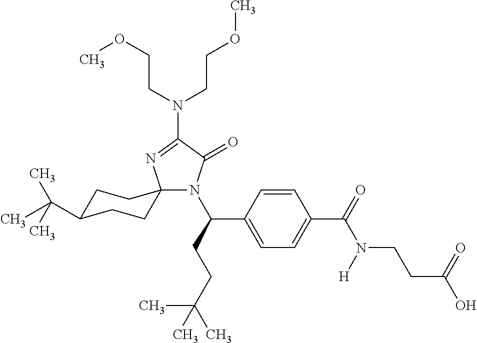 [SMILES: COCCN(CCOC)C1=NC2(CCC(C(C)(C)C)CC2)N([C@H](CCC(C)(C)C)c2ccc(C(=O)NCCC(=O)O)cc2)C1=O] | 3 | 1.08 | 629 |
| A-79 | 10.21 | 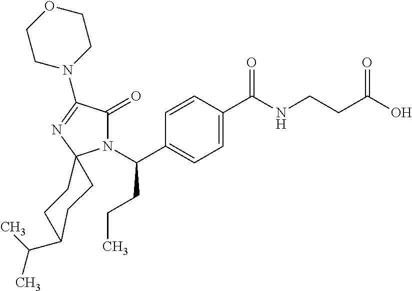 [SMILES: CCC[C@H](c1ccc(C(=O)NCCC(=O)O)cc1)N1C(=O)C(N2CCOCC2)=NC12CCC(C(C)C)CC2] | 1 | 1.97 | 527 |
| I-6 | 10.22 | 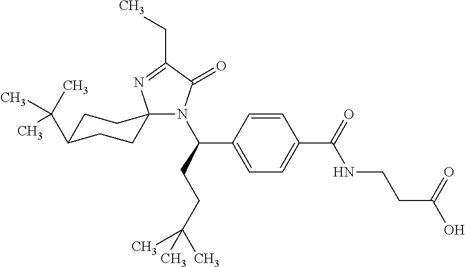 [SMILES: CCC1=NC2(CCC(C(C)(C)C)CC2)N([C@H](CCC(C)(C)C)c2ccc(C(=O)NCCC(=O)O)cc2)C1=O] | 1 | 2.44 | 526 |
| I-7 | 10.23 | 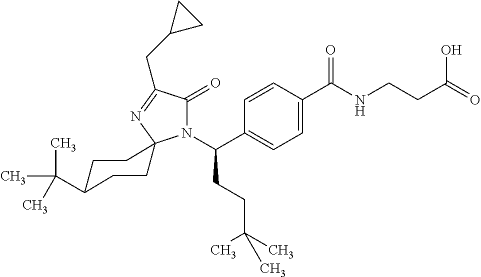 [SMILES: CC(C)(C)CC[C@H](c1ccc(C(=O)NCCC(=O)O)cc1)N1C(=O)C(CC2CC2)=NC12CCC(C(C)(C)C)CC2] | 6 | 16.95 | 552 |
| I-8 | 10.24 | 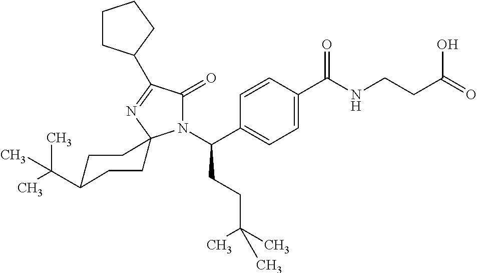 [SMILES: CC(C)(C)CC[C@H](c1ccc(C(=O)NCCC(=O)O)cc1)N1C(=O)C(C2CCCC2)=NC12CCC(C(C)(C)C)CC2] | 6 | 21.24 | 566 |
| I-9 | 10.25 | 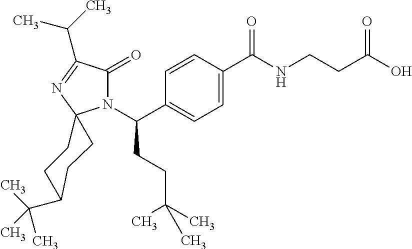 [SMILES: CC(C)C1=NC2(CCC(C(C)(C)C)CC2)N([C@H](CCC(C)(C)C)c2ccc(C(=O)NCCC(=O)O)cc2)C1=O] | 1 | 2.55 | 540 |
| I-10 | 10.26 | 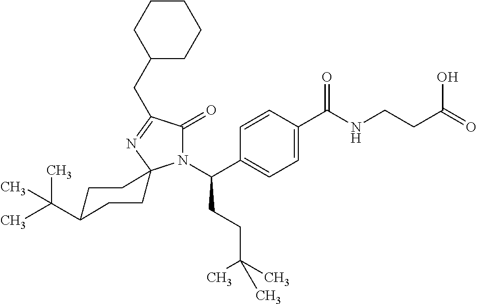 [SMILES: CC(C)(C)CC[C@H](c1ccc(C(=O)NCCC(=O)O)cc1)N1C(=O)C(CC2CCCCC2)=NC12CCC(C(C)(C)C)CC2] | 6 | 22.88 | 594 |
| I-11 | 10.27 | 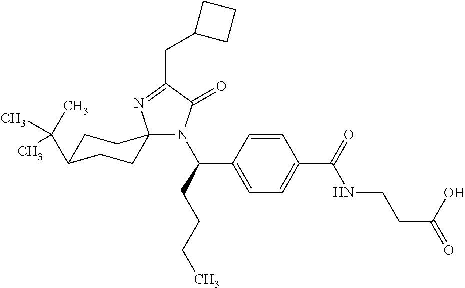 [SMILES: CCCC[C@H](c1ccc(C(=O)NCCC(=O)O)cc1)N1C(=O)C(CC2CCC2)=NC12CCC(C(C)(C)C)CC2] | 6 | 17.06 | 538 |
| I-12 | 10.28 | 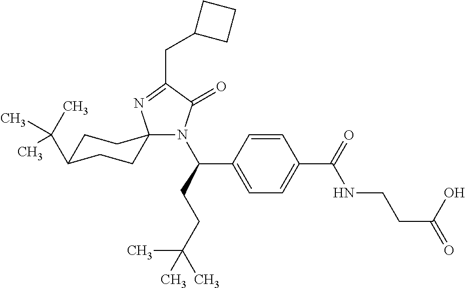 [SMILES: CC(C)(C)CC[C@H](c1ccc(C(=O)NCCC(=O)O)cc1)N1C(=O)C(CC2CCC2)=NC12CCC(C(C)(C)C)CC2] | 6 | 19.56 | 566 |
| I-13 | 10.29 | 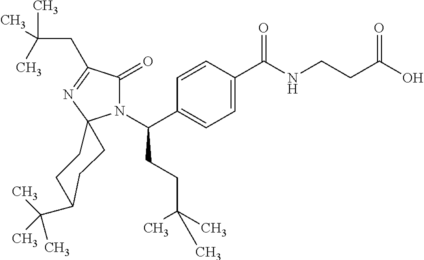 [SMILES: CC(C)(C)CC[C@H](c1ccc(C(=O)NCCC(=O)O)cc1)N1C(=O)C(CC(C)(C)C)=NC12CCC(C(C)(C)C)CC2] | 6 | 20.03 | 568 |
| I-14 | 10.30 | 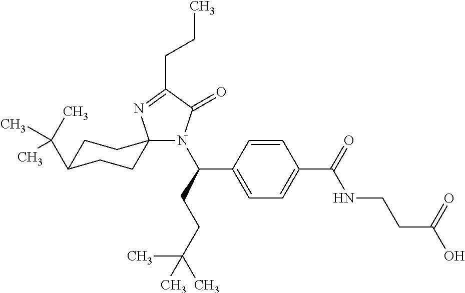 [SMILES: CCCC1=NC2(CCC(C(C)(C)C)CC2)N([C@H](CCC(C)(C)C)c2ccc(C(=O)NCCC(=O)O)cc2)C1=O] | 6 | 17.31 | 540 |
| I-15 | 10.31 | 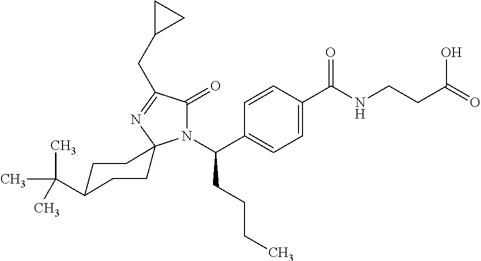 [SMILES: CCCC[C@H](c1ccc(C(=O)NCCC(=O)O)cc1)N1C(=O)C(CC2CC2)=NC12CCC(C(C)(C)C)CC2] | 6 | 14.73 | 524 |
| C-5 | 10.32 | 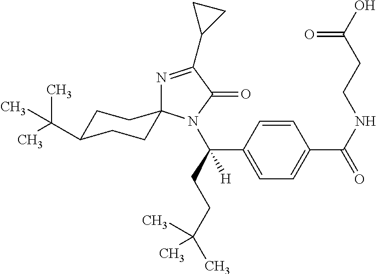 [SMILES: CC(C)(C)CC[C@H](c1ccc(C(=O)NCCC(=O)O)cc1)N1C(=O)C(C2CC2)=NC12CCC(C(C)(C)C)CC2] | 1 | 2.55 | 538 |
| I-17 | 10.33 | 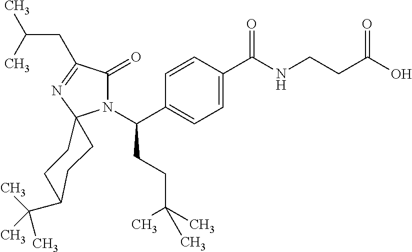 [SMILES: CC(C)CC1=NC2(CCC(C(C)(C)C)CC2)N([C@H](CCC(C)(C)C)c2ccc(C(=O)NCCC(=O)O)cc2)C1=O] | 6 | 18.39 | 554 |
| I-18 | 10.34 | 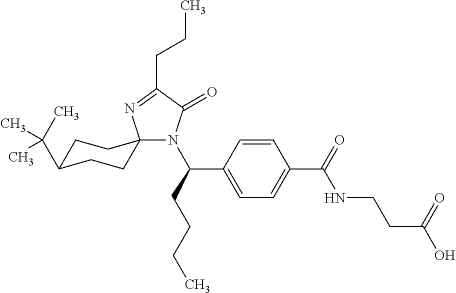 [SMILES: CCCC[C@H](c1ccc(C(=O)NCCC(=O)O)cc1)N1C(=O)C(CCC)=NC12CCC(C(C)(C)C)CC2] | 6 | 15.01 | 512 |
| I-19 | 10.35 | 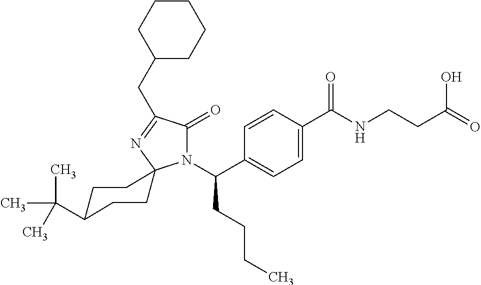 [SMILES: CCCC[C@H](c1ccc(C(=O)NCCC(=O)O)cc1)N1C(=O)C(CC2CCCCC2)=NC12CCC(C(C)(C)C)CC2] | 6 | 19.75 | 566 |
| I-16 | 10.36 | 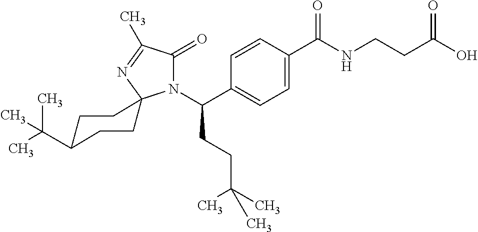 [SMILES: CC1=NC2(CCC(C(C)(C)C)CC2)N([C@H](CCC(C)(C)C)c2ccc(C(=O)NCCC(=O)O)cc2)C1=O] | 5 | 18.78 (15) | 512 |
| I-20 | 10.37 | 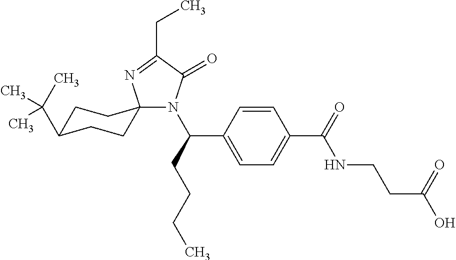 [SMILES: CCCC[C@H](c1ccc(C(=O)NCCC(=O)O)cc1)N1C(=O)C(CC)=NC12CCC(C(C)(C)C)CC2] | 6 | 13.94 | 498 |
| I-21 | 10.38 | 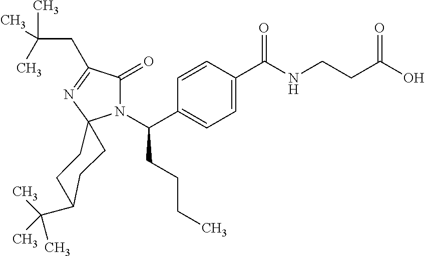 [SMILES: CCCC[C@H](c1ccc(C(=O)NCCC(=O)O)cc1)N1C(=O)C(CC(C)(C)C)=NC12CCC(C(C)(C)C)CC2] | 6 | 17.39 | 540 |
| I-5 | 10.39 | 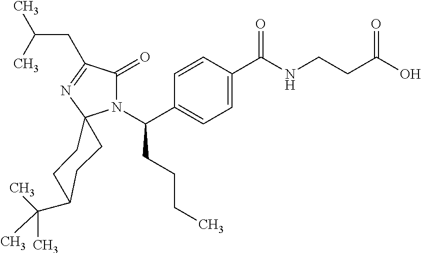 [SMILES: CCCC[C@H](c1ccc(C(=O)NCCC(=O)O)cc1)N1C(=O)C(CC(C)C)=NC12CCC(C(C)(C)C)CC2] | 6 | 16.07 | 526 |
| I-22 | 10.40 | 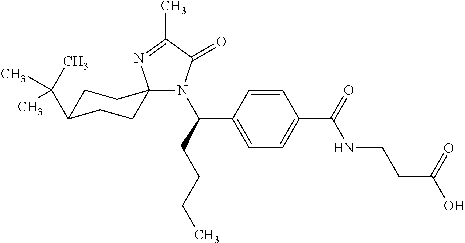 [SMILES: CCCC[C@H](c1ccc(C(=O)NCCC(=O)O)cc1)N1C(=O)C(C)=NC12CCC(C(C)(C)C)CC2] | 6 | 11.66 | 484 |
| O-1 | 10.41 | 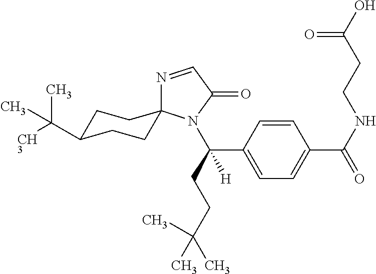 [SMILES: CC(C)(C)CC[C@H](c1ccc(C(=O)NCCC(=O)O)cc1)N1C(=O)C=NC12CCC(C(C)(C)C)CC2] | 1 | 2.34 | 498 |
| B-9 | 10.42 | 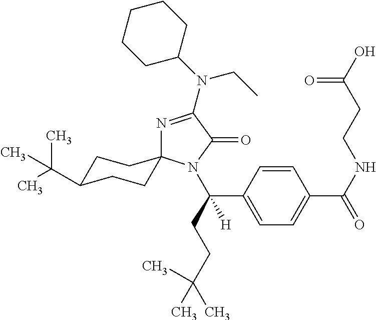 [SMILES: CCN(C1=NC2(CCC(C(C)(C)C)CC2)N([C@H](CCC(C)(C)C)c2ccc(C(=O)NCCC(=O)O)cc2)C1=O)C1CCCCC1] | 1 | 2.28 | 624 |
| B-10 | 10.43 | 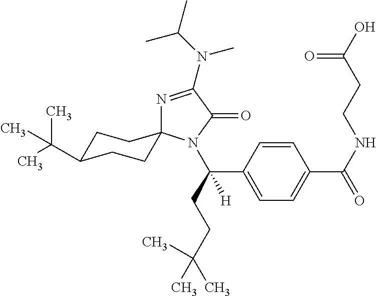 [SMILES: CC(C)N(C)C1=NC2(CCC(C(C)(C)C)CC2)N([C@H](CCC(C)(C)C)c2ccc(C(=O)NCCC(=O)O)cc2)C1=O] | 1 | 2.17 | 570 |
| A-94 | 10.44 | 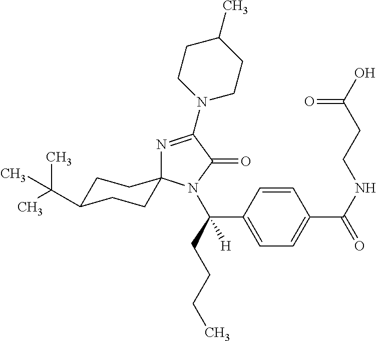 [SMILES: CCCC[C@H](c1ccc(C(=O)NCCC(=O)O)cc1)N1C(=O)C(N2CCC(C)CC2)=NC12CCC(C(C)(C)C)CC2] | 5 | 16.50 (20) | 567 |
| A-95 | 10.45 | 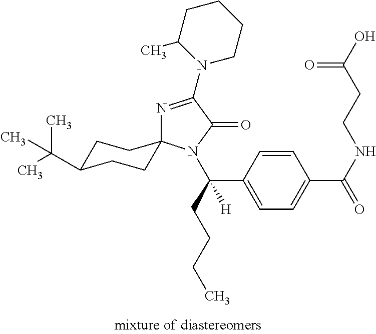 [SMILES: CCCC[C@H](c1ccc(C(=O)NCCC(=O)O)cc1)N1C(=O)C(N2CCCCC2C)=NC12CCC(C(C)(C)C)CC2] | 5 | 16.31 (20) | 567 |
| A-96 | 10.46 | 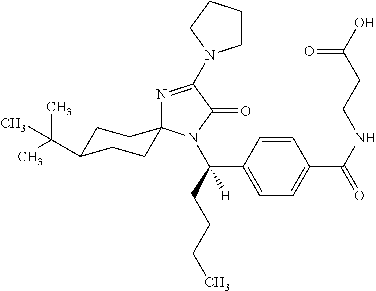 [SMILES: CCCC[C@H](c1ccc(C(=O)NCCC(=O)O)cc1)N1C(=O)C(N2CCCC2)=NC12CCC(C(C)(C)C)CC2] | 5 | 13.99 (20) | 539 |
| A-97 | 10.47 | 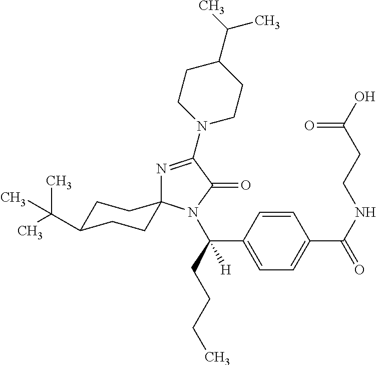 [SMILES: CCCC[C@H](c1ccc(C(=O)NCCC(=O)O)cc1)N1C(=O)C(N2CCC(C(C)C)CC2)=NC12CCC(C(C)(C)C)CC2] | 5 | 16.49 (20) | 595 |
| B-11 | 10.48 | 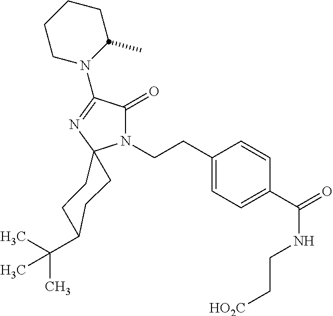 | | | |
| B-12 | 10.49 | 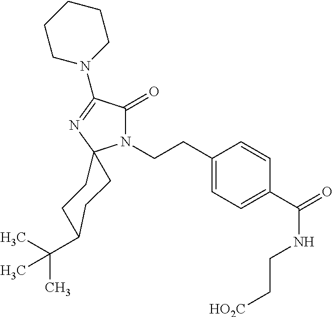 [SMILES: CC(C)(C)C1CCC2(CC1)N=C(N1CCCCC1)C(=O)N2CCc1ccc(C(=O)NCCC(=O)O)cc1] | 1 | 1.97 | 511 |

TABLE 11
Using the requisite starting material, and a method similar to that outlined in
Scheme P, the following compounds were prepared:

| | | | LCMS | | |
|---|---|---|---|---|---|
| Benzoic | | | | Ret | |
| acid | Ex. | Structure | LC | (min) | [M + H]⁺ |
| B-4 | 11.1 | 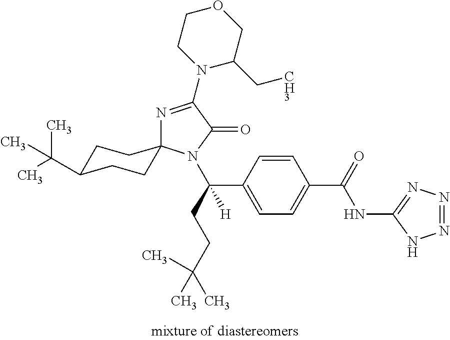 [SMILES: CCC1COCCN1C1=NC2(CCC(C(C)(C)C)CC2)N([C@H](CCC(C)(C)C)c2ccc(C(=O)Nc3nnn[nH]3)cc2)C1=O] | 1 | 2.19 | 607 |
| A-8 | 11.2 | 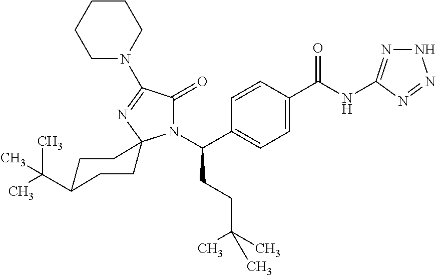 [SMILES: CC(C)(C)CC[C@H](c1ccc(C(=O)Nc2nn[nH]n2)cc1)N1C(=O)C(N2CCCCC2)=NC12CCC(C(C)(C)C)CC2] | 5 | 16.27 (20) | 577 |

### LCMS Conditions

### LC-1:

LCMS spectra were obtained on an Agilent 6140 Quadrupole LCMS, using a Zorbax SB-C-18 column (3.0 mm×50 mm, 1.8 micron) and a flow rate of 1.0 mL/min.

### Mobile Phase:

Solvent A: Water with 0.1% trifluoroacetic acid by volume.

Solvent B: Acetonitrile with 0.1% trifluoroacetic acid by volume.

### Gradient Table Time:

0 min=10% Solvent B

0.3 min=10% Solvent B

1.5 min=95% Solvent B

2.7 min=95% Solvent B

2.8 min=10% Solvent B

Stop Time=3.60 min.

Post Time=0.7 min.

### Column Temperature: 50° C.

### LC-2:

Column: Gemini C-18, 50×4.6 mm, 5 micron, obtained from Phenomenex. Mobile phase: A: 0.05% Trifluoroacetic acid in water B: 0.05% Trifluofloacetic acid in acetonitrile Gradient: 90:10 to 5:95 (A:B) over 5 min. Flow rate: 1.0 mL/min UV detection: 254 nm. ESI-MS: Electro Spray Ionization Liquid chromatography-mass spectrometry (ESI-LC/MS) was performed on a PE SCIEX API-150EX, single quadrupole mass spectrometer.

### LC-3:

LCMS spectra were obtained on an Agilent 6140 Quadrupole LCMS, using a Zorbax SB-C-18 column (Rapid Resolution Cartridge, 2.1 mm×30 mm, 3.5 micron) and a flow rate of 2.0 mL/min.

### Mobile Phase:

Solvent A: Water with 0.1% trifluoroacetic acid by volume.

Solvent B: Acetonitrile with 0.1% trifluoroacetic acid by volume.

### Gradient Table Time:

0.01 min=10% Solvent B

1.01 min=95% Solvent B

1.37 min=95% Solvent B

1.38 min=10% Solvent B

Stop Time=1.70 min.

LC-5: HPLC conditions for the retention time were as follows: Column: Luna C18 100A, 5 μM: A: 0.025% TFA in water B: 0.025% TFA in acetonitrile: Gradient: 98:2 to 2:98 (A:B) over indicated time in parenthesis (below retention time provided in corresponding Table followed by a 2 minute gradient back to 98:2 (A:B)). Flow rate: 1.0 ml/min UV detection: 254 nm. Mass spec were obtained by one of the following methods: a) Multimode (ESI and APCI). b) ESI

LC-6: HPLC conditions for the retention time were as follows: Column: Luna C18 100A, 5 μM: A: 0.025% TFA in water B: 0.025% TFA in acetonitrile: Gradient: 98:2 to 15:85 (A:B) over 5 min., then gradient to 2:98 (A:B) over 10 min., then hold at 2:98 (A:B) for 19 min. This is followed by a 2 minute gradient back to 98:2 (A:B). Flow rate: 1.0 ml/min UV detection: 254 nm. Mass spectra were obtained by one of the following methods: a) Multimode (ESI and APCI). b) ESI.

### Biological Assays

The ability of the compounds of the invention to inhibit the binding of glucagon and their utility in treating or preventing type 2 diabetes mellitus and related conditions can be demonstrated by the following in vitro assays.

### Glucagon Receptor Binding Assay

Recombinant human glucagon receptor (huGlucR) membranes and mouse glucagon receptor (mGlucR) membranes were prepared in-house from huGlucR/clone 103c/CHO and mouse liver tissue, respectively. 0.03 ug/li huGluR membranes (or 0.5 ug/ml mGlucR) was incubated in assay buffer containing 0.05 nM ¹²⁵I-Glucagon (Perkin Elmer, NEX 207) and varying concentrations of antagonist at room temperature for 60 to 90 min. (assay buffer: 50 mM HEPES, 1 mM MgCl2, 1 mM CaCl2, 1 mg/ml BSA, COMPLETE protease inhibitor cocktail, pH 7.4). The total volume of the assay was 200 μl with 4% final DMSO concentration. The assay was performed at room temperature using 96-deep well plate. Compound 4c, racemic diastereomer 1 (D1), (1.0 μM final concentration), described by G. H. Ladouceur et al. in Bioorganic and Medicinal Chemistry Letters, 12 (2002), 3421-3424, was used to determine non-specific binding. Following incubation, the reaction was stopped by rapid filtration through Unfilter-96 GF/C glass fiber filter plates (Perkin Elmer) pre-soaked in 0.5% polyethyleneimine. The filtrate was washed using 50 mM Tris-HCl, pH 7.4. Dried filter plates containing bound radioactivity were counted in the presence of scintillation fluid (Microscint 0, Perkin-Elmer) using a Topcount scintillation counter. Data was analyzed using the software program Prism (GraphPad). IC₅₀ values were calculated using non-linear regression analysis assuming single site competition.

Inhibition of Glucagon-Stimulated Intracellular cAMP Assay

Chinese hamster ovary (CHO) cells expressing the recombinant human glucagon receptor were harvested with the aid of non-enzymatic cell dissociation solution (GIBCO 13151-014). The cells were then pelleted and suspended in the stimulation buffer (1×HBSS, 5 mM Hepes, 0.1% BSA, pH7.4 in presence of complete protease inhibitor and phosphodiesterase inhibitor). The adenylate cyclase assay was conducted following the LANCE cAMP Kit (Perkin Elmer, AD0262) instructions. Briefly, cells were preincubated with anti-cAMP antibody in the stimulation buffer with a final concentration of 3% DMSO for 30 minutes and then stimulated with 300 pM glucagon for 45 minutes. The reaction was stopped by incubating with the detection buffer containing Europium chelate of the Eu-SA/Biotin-cAMP tracer for 20 hours. The fluorescence intensity emitted from the assay was measured at 665 nm using PheraStar instruments. Basal activity (100% inhibition) was determined using the DMSO control and 0% inhibition was defined as cAMP stimulation produced by 300 pM glucagon. Standard cAMP concentrations were conducted concurrently for conversion of fluorescence signal to cAMP level. Data was analyzed using GraphPad Prism. IC₅₀ values were calculated using non-linear regression analysis assuming single site competition. IC₅₀ values for all of the compounds of the invention shown in the examples measured less than about 10 μM in this functional assay. Some of the compounds of the invention shown in the examples measured less than about 5 μM in this assay; other examples measured less than about 500 nM; others less than about 100 nM.

The IC₅₀ results in the cAMP assay are given below for the indicated compounds.

| | | IC₅₀ |
|---|---|---|
| Example | Structure | (nM) |
| 10.4 | 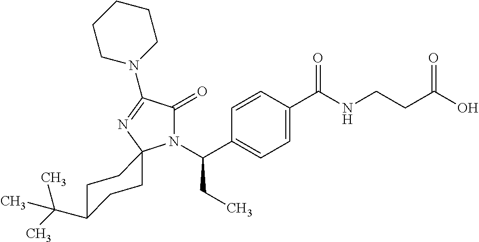 [SMILES: CC[C@H](c1ccc(C(=O)NCCC(=O)O)cc1)N1C(=O)C(N2CCCCC2)=NC12CCC(C(C)(C)C)CC2] | 209 |
| 9.99 | 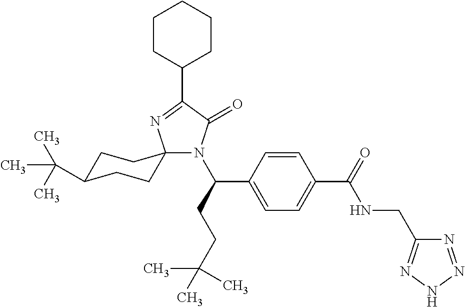 [SMILES: CC(C)(C)CC[C@H](c1ccc(C(=O)NCc2nn[nH]n2)cc1)N1C(=O)C(C2CCCCC2)=NC12CCC(C(C)(C)C)CC2] | 290 |

In another embodiment, the present invention provides a pharmaceutical composition comprising a compound of the invention described above in combination with a pharmaceutically acceptable carrier.

In another embodiment, the present invention provides a method for inhibiting glucagon receptors comprising exposing an effective amount of a compound or a composition comprising a compound of the invention to glucagon receptors. In one embodiment, said glucagon receptors are part of a glucagon receptor assay. Non-limiting examples of such assays include glucagon receptor assays and glucagon-stimulated intracellular cAMP formation assays such as those described above. In one embodiment, said glucagon receptors are expressed in a population of cells. In one embodiment, the population of cells is in in vitro. In one embodiment, the population of cells is in ex vivo. In one embodiment, the population of cells is in a patient.

Methods of Treatment, Compositions, and Combination Therapy

In another embodiment, the present invention provides a method of treating type 2 diabetes mellitus in a patient in need of such treatment comprising administering to said patient a compound of the invention or a composition comprising a compound of the invention in an amount effective to treat type 2 diabetes mellitus.

In another embodiment, the present invention provides a method of delaying the onset of type 2 diabetes mellitus in a patient in need of such treatment comprising administering to said patient a compound of the invention or a composition comprising a compound of the invention in an amount effective to delay the onset of type 2 diabetes mellitus.

In another embodiment, the present invention provides a method of treating hyperglycemia, diabetes, or insulin resistance in a patient in need of such treatment comprising administering to said patient a compound of the invention, or a composition comprising a compound of the invention, in an amount that is effective to treat hyperglycemia, diabetes, or insulin resistance.

In another embodiment, the present invention provides a method of treating non-insulin dependent diabetes mellitus in a patient in need of such treatment comprising administering to said patient an anti-diabetic effective amount of a compound of the invention or a composition comprising an effective amount of a compound of the invention.

In another embodiment, the present invention provides a method of treating obesity in a patient in need of such treatment comprising administering to said patient a compound of the invention or a composition comprising a compound of the invention in an amount that is effective to treat obesity.

In another embodiment, the present invention provides a method of treating one or more conditions associated with Syndrome X (also known as metabolic syndrome, metabolic syndrome X, insulin resistance syndome, Reaven's syndrome) in a patient in need of such treatment comprising administering to said patient a compound of the invention or a composition comprising an effective amount of a compound of the invention in an amount that is effective to treat Syndrome X.

In another embodiment, the present invention provides a method of treating a lipid disorder in a patient in need of such treatment comprising administering to said patient a compound of the invention, or a composition comprising a compound of the invention, in an amount that is effective to treat said lipid disorder. Non-limiting examples of such lipid disorders include: dyslipidemia, hyperlipidemia, hypertriglyceridemia, hypercholesterolemia, low HDL and high LDL, and metabolic syndrome.

In another embodiment, the present invention provides a method of treating atherosclerosis in a patient in need of such treatment comprising administering to said patient a compound of the invention or a composition comprising a compound of the invention, in an amount effective to treat atherosclerosis.

In another embodiment, the present invention provides a method of delaying the onset of, or reducing the risk of developing, atherosclerosis in a patient in need of such treatment comprising administering to said patient a compound of the invention or a composition comprising a compound of the invention, in an amount effective to delay the onset of, or reduce the risk of developing, atherosclerosis.

In another embodiment, the present invention provides a method of treating a condition or a combination of conditions selected from hyperglycemia, low glucose tolerance, insulin resistance, obesity, abdominal obesity, lipid disorders, dyslipidemia, hyperlipidemia, hypertriglyceridemia, hypercholesterolemia, low HDL levels, high LDL levels, atherosclerosis, atherosclerosis and its sequelae, vascular restenosis, pancreatitis, neurodegenerative disease, retinopathy, nephropathy, neuropathy, Syndrome X and other conditions where insulin resistance is a component, in a patient in need thereof, comprising administering to said patient a compound of the invention, or a composition comprising a compound of the invention, in an amount that is effective to treat said condition or conditions.

In another embodiment, the present invention provides a method of delaying the onset of a condition or a combination of conditions selected from hyperglycemia, low glucose tolerance, insulin resistance, obesity, abdominal obesity, lipid disorders, dyslipidemia, hyperlipidemia, hypertriglyceridemia, hypercholesterolemia, low HDL levels, high LDL levels, atherosclerosis, atherosclerosis and its sequelae, vascular restenosis, pancreatitis, neurodegenerative disease, retinopathy, nephropathy, neuropathy, Syndrome X and other conditions where insulin resistance is a component, in a patient in need thereof, comprising administering to said patient a compound of the invention, or a composition comprising a compound of the invention, in an amount that is effective to delay the onset said condition or conditions.

In another embodiment, the present invention provides a method of reducing the risk of developing a condition or a combination of conditions selected from hyperglycemia, low glucose tolerance, insulin resistance, obesity, abdominal obesity, lipid disorders, dyslipidemia, hyperlipidemia, hypertriglyceridemia, hypercholesterolemia, low HDL levels, high LDL levels, atherosclerosis, atherosclerosis and its sequelae, vascular restenosis, pancreatitis, neurodegenerative disease, retinopathy, nephropathy, neuropathy, Syndrome X and other conditions where insulin resistance or hyperglycemia is a component, in a patient in need thereof, comprising administering to said patient a compound of the invention, or a composition comprising a compound of the invention, in an amount that is effective to reduce the risk of developing said condition or conditions.

In another embodiment, the present invention provides a method of treating a condition selected from type 2 diabetes mellitus, hyperglycemia, low glucose tolerance, insulin resistance, obesity, abdominal obesity, lipid disorders, dyslipidemia, hyperlipidemia, hypertriglyceridemia, hypercholesterolemia, low HDL levels, high LDL levels, atherosclerosis, atherosclerosis and its sequelae, vascular restenosis, pancreatitis, neurodegenerative disease, retinopathy, nephropathy, neuropathy, Syndrome X and other conditions where insulin resistance is a component, in a patient in need thereof, comprising administering to said patient effective amounts of a compound of the invention and one or more additional active agents.

Non-limiting examples of such additional active agents include the following:

DPP-IV inhibitors. Non-limiting examples of DPP-IV inhibitors include alogliptin (Takeda), linagliptin, saxagliptin (Brystol-Myers Squibb), sitagliptin (Januvia™, Merck), vildagliptin (Galvus™, Novartis), denagliptin (GlaxoSmithKline), ABT-279 and ABT-341 (Abbott), ALS-2-0426 (Alantos), AR1-2243 (Arisaph), BI-A and BI-B (Boehringer Ingelheim), SYR-322 (Takeda), compounds disclosed in U.S. Pat. No. 6,699,871, MP-513 (Mitsubishi), DP-893 (Pfizer), RO-0730699 (Roche) and combinations thereof. Non-limiting examples of such combinations include Janumet™, a combination of sitagliptin/metformin HCl (Merck).

Insulin sensitizers. Non-limiting examples of insulin sensitizers include PPAR agonists and biguanides. Non-limiting examples of PPAR agonists include glitazone and thiaglitazone agents such as rosiglitazone, rosiglitazone maleate (AVANDIA™, GlaxoSmithKline), pioglitazone, pioglitazone hydrochloride (ACTOS™, Takeda), ciglitazone and MCC-555 (Mitstubishi Chemical Co.), troglitazone and englitazone. Non-limiting example of biguanides include phenformin, metformin, metformin hydrochloride (such as GLUCOPHAGE®, Bristol-Myers Squibb), metformin hydrochloride with glyburide (such as GLUCOVANCE™, Bristol-Myers Squibb) and buformin. Other non-limiting examples of insulin sensitizers include PTP-1 B inhibitors; and glucokinase activators, such as miglitol, acarbose, and voglibose.

Insulin and insulin mimetics. Non-limiting examples of orally administrable insulin and insulin containing compositions include AL-401 (Autoimmune), and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference.

Sulfonylureas and other insulin secretagogues. Non-limiting examples of sulfonylureas and other secretagogues include glipizide, tolbutamide, glyburide, glimepiride, chlorpropamide, acetohexamide, gliamilide, gliclazide, glibenclamide, tolazamide, GLP-1, GLP-1 mimetics, exendin, GIP, secretin, nateglinide, meglitinide, glibenclamide, and repaglinide. Non-limiting examples of GLP-1 mimetics include Byetta™ (exenatide), liraglutide, CJC-1131 (ConjuChem), exenatide-LAR (Amylin), BIM-51077 (Ipsen/LaRoche), ZP-10 (Zealand Pharmaceuticals), and compounds disclosed in International Publication No. WO 00/07617.

Glucosidase inhibitors and alpha glucosidase inhibitors.

Glucagon receptor antagonists other than compounds of the invention.

Hepatic glucose output lowering agents other than a glucagon receptor antagonist. Non-limiting examples of hepatic glucose output lowering agents include Glucophage and Glucophage XR.

An antihypertensive agent. Non-limiting examples of antihypertensive agents include beta-blockers and calcium channel blockers (for example diltiazem, verapamil, nifedipine, amlopidine, and mybefradil), ACE inhibitors (for example captopril, lisinopril, enalapril, spirapril, ceranopril, zefenopril, fosinopril, cilazoprif, and quinapril), AT-1 receptor antagonists (for example losartan, irbesartan, and valsartan), renin inhibitors and endothelin receptor antagonists (for example sitaxsentan).

A meglitinide. Non-limiting examples of meglitinides useful in the present methods for treating diabetes include repaglinide and nateglinide.

An agent that blocks or slows the breakdown of starches or sugars in viva Non-limiting examples of antidiabetic agents that slow or block the breakdown of starches and sugars in vivo include alpha-glucosidase inhibitors and certain peptides for increasing insulin production; Alpha-glucosidase inhibitors (which help the body to lower blood sugar by delaying the digestion of ingested carbohydrates, thereby resulting in a smaller rise in blood glucose concentration following meals). Non-limiting examples of alpha-glucosidase inhibitors include acarbose; miglitol; camiglibose; certain polyamines as disclosed in WO 01/47528 (incorporated herein by reference); and voglibose.

Peptides for increasing insulin production. Non-limiting examples of suitable peptides for increasing insulin production including amlintide (CAS Reg. No. 122384-88-7, Amylin); pramlintide, exendin, certain compounds having Glucagon-like peptide-1 (GLP-1) agonistic activity as disclosed in WO 00/07617 (incorporated herein by reference).

A histamine H₃ receptor antagonist. Non-limiting examples of histamine H₃ receptor antagonist agents include the following compound:

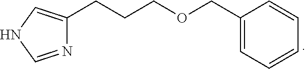 [SMILES: c1ccc(COCCCc2c[nH]cn2)cc1]

A sodium glucose uptake transporter 2 (SGLT-2) inhibitor. Non-limiting examples of SGLT-2 inhibitors useful in the present methods include dapagliflozin and sergllfiozin, AVE2268 (Sanofi-Aventis) and T-1095 (Tanabe Seiyaku).

PACAP (pituitary adenylate cyclase activating polypeptide agonists) and PACAP mimetics.

Cholesterol lowering agents. Non-limiting examples of cholesterol lowering agents include HMG-CoA reducatase inhibitors, sequestrants, nicotinyl alcohol, nicotinic acid and salts thereof, PPAR alpha agonists, PPAR alpha/gamma dual agonists, inhibitors of cholesterol absorption (such as ezetimibe (Zetia®)), combinations of HMG-CoA reductase inhibitors and cholesterol absorption agents (such as Vytorin®), acyl CoA:cholesterol acyltransferase inhibitors, anti-oxidants, LXR modulators, and CETP (cholesterolester transfer protein) inhibitors such as Torcetrapib™ (Pfizer) and Anacetrapib™ (Merck).

Agents capable of raising serum HDL cholesterol levels. Non-limiting examples include niacin (vitamin B-3), such as Niaspan™ (Kos). Niacin may be administered alone or optionally combined with one or more additional active agents such as: niacin/lovastatin (Advicor™, Abbott), niacin/simvastatin (Simcor™, Abbott), and/or niacin/aspirin.

PPAR delta agonists.

Antiobesity agents. Non-limiting examples of anti-obesity agents useful in the present methods for treating diabetes include a 5-HT2C agonist, such as lorcaserin; a neuropeptide γ antagonist; an MCR4 agonist; an MCH receptor antagonist; a protein hormone, such as leptin or adiponectin; an AMP kinase activator; and a lipase inhibitor, such as orlistat.

Ileal bile acid transporter inhibitors.

Anti-inflammatory agents, such as NSAIDs. Non-limiting examples of NSAIDS include a salicylate, such as aspirin, amoxiprin, benorilate or diflunisal; an arylalkanoic acid, such as diclofenac, etodolac, indometacin, ketorolac, nabumetone, sulindac or tolmetin; a 2-arylpropionic acid (a “profen”), such as ibuprofen, carprofen, fenoprofen, flurbiprofen, loxoprofen, naproxen, tiaprofenic acid or suprofen; a fenamic acid, such as mefenamic acid or meclofenamic acid; a pyrazolidine derivative, such as phenylbutazone, azapropazone, metamizole or oxyphenbutazone; a coxib, such as celecoxib, etoricoxib, lumiracoxib or parecoxib; an oxicam, such as piroxicam, lornoxicam, meloxicam or tenoxicam; or a sulfonanilide, such as nimesulide.

Anti-pain medications, including NSAIDs as discussed above, and opiates. Non-limiting examples of opiates include an anilidopiperidine, a phenylpiperidine, a diphenylpropylamine derivative, a benzomorphane derivative, an oripavine derivative and a morphinane derivative. Additional illustrative examples of opiates include morphine, diamorphine, heroin, buprenorphine, dipipanone, pethidine, dextromoramide, alfentanil, fentanyl, remifentanil, methadone, codeine, dihydrocodeine, tramadol, pentazocine, vicodin, oxycodone, hydrocodone, percocet, percodan, norco, dilaudid, darvocet or lorcet.

Antidepressants. Non-limiting examples of tricyclic antidepressants useful in the present methods for treating pain include amitryptyline, carbamazepine, gabapentin or pregabalin.

Protein tyrosine phosphatase-1 B (PIP-1B) inhibitors.

CB1 antagonists/inverse agonists. Non-limiting examples of CB1 receptor antagonists and inverse agonists include rimonabant and those disclosed in WO03/077847A2, published Sep. 25, 2003, WO05/000809, published Jan. 6, 2005, and WO2006/060461, published Jun. 8, 2006.

In another embodiment, the present invention provides a method of treating a condition selected from hypercholesterolemia, atherosclerosis, low HDL levels, high

LDL levels, hyperlipidemia, hypertriglyceridemia, and dyslipidemia, in a patient in need of such treatment, comprising administering to the patient a therapeutically effective amount or amounts of a compound of the invention, or a composition comprising a compound of the invention, and an HMG-CoA reductase inhibitor.

In another embodiment, the present invention provides a method of treating a condition selected from hypercholesterolemia, atherosclerosis, low HDL levels, high LDL levels, hyperlipidemia, hypertriglyceridemia, and dyslipidemia, in a patient in need of such treatment, comprising administering to the patient a therapeutically effective amount or amounts of a compound of the invention, or a composition comprising a compound of the invention, and an HMG-CoA reductase inhibitor, wherein the HMG-CoA reductase inhibitor is a statin.

In another embodiment, the present invention provides a method of treating a condition selected from hypercholesterolemia, atherosclerosis, low HDL levels, high LDL levels, hyperlipidemia, hypertriglyceridemia, and dyslipidemia, in a patient in need of such treatment, comprising administering to the patient a therapeutically effective amount or amounts of a compound of the invention, or a composition comprising a compound of the invention, and an HMG-CoA reductase inhibitor, wherein the HMG-CoA reductase inhibitor is a statin selected from lovastatin, simvastatin, pravastatin, fluvastatin, atorvastatin, itavastatin, ZD-4522, and rivastatin.

In another embodiment, the present invention provides a method of reducing the risk of developing, or delaying the onset of, a condition selected from hypercholesterolemia, atherosclerosis, low HDL levels, high LDL levels, hyperlipidemia, hypertriglyceridemia, and dyslipidemia, in a patient in need of such treatment, comprising administering to the patient a therapeutically effective amount or amounts of a compound of the invention, or a composition comprising a compound of the invention, and an HMG-CoA reductase inhibitor.

In another embodiment, the present invention provides a method of reducing the risk of developing, or delaying the onset of, a condition selected from hypercholesterolemia, atherosclerosis, low HDL levels, high LDL levels, hyperlipidemia, hypertriglyceridemia, and dyslipidemia, in a patient in need of such treatment, comprising administering to the patient a therapeutically effective amount or amounts of a compound of the invention, or a composition comprising a compound of the invention, and an HMG-CoA reductase inhibitor, wherein the HMG-CoA reductase inhibitor is a statin.

In another embodiment, the present invention provides a method of reducing the risk of developing, or delaying the onset of, a condition selected from hypercholesterolemia, atherosclerosis, low HDL levels, high LDL levels, hyperlipidemia, hypertriglyceridemia, and dyslipidemia, in a patient in need of such treatment, comprising administering to the patient a therapeutically effective amount or amounts of a compound of the invention, or a composition comprising a compound of the invention, and an HMG-CoA reductase inhibitor, wherein the HMG-CoA reductase inhibitor is a statin selected from lovastatin, simvastatin, pravastatin, fluvastatin, atorvastatin, itavastatin, ZD-4522, and rivastatin.

In another embodiment, the present invention provides a method of reducing the risk of developing, or delaying the onset of atherosclerosis, high LDL levels, hyperlipidemia, and dyslipidemia, in a patient in need of such treatment, comprising administering to the patient a therapeutically effective amount or amounts of a compound of the invention, or a composition comprising a compound of the invention, and a cholesterol absorption inhibitor, optionally in further combination with a statin.

In another embodiment, the present invention provides a method of reducing the risk of developing, or delaying the onset of atherosclerosis, high LDL levels, hyperlipidemia, and dyslipidemia, in a patient in need of such treatment, comprising administering to the patient a therapeutically effective amount or amounts of a compound of the invention, or a composition comprising a compound of the invention, and a cholesterol absorption inhibitor, optionally in further combination with one or more statins, wherein the cholesterol absorption inhibitor is selected from ezetimibe, ezetimibe/simvastatin combination (Vytorin®), and a stanol.

In another embodiment, the present invention provides a pharmaceutical composition comprising (1) a compound according to the invention; (2) one or more compounds or agents selected from DPP-IV inhibitors, insulin sensitizers, insulin and insulin mimetics, a sulfonylurea, an insulin secretagogue, a glucosidase inhibitor, an alpha glucosidase inhibitor, a glucagon receptor antagonists other than a compound of the invention, a hepatic glucose output lowering agent other than a glucagon receptor antagonist, an antihypertensive agent, a meglitinide, an agent that blocks or slows the breakdown of starches or sugars in vivo, an alpha-glucosidase inhibitor, a peptide capable of increasing insulin production, a histamine H₃ receptor antagonist, a sodium glucose uptake transporter 2 (SGLT-2) inhibitor, a peptide that increases insulin production, a GIP cholesterol lowering agent, a PACAP, a PACAP mimetic, a PACAP receptor 3 agonist, a cholesterol lowering agent, a PPAR delta agonist, an antiobesity agent, an ileal bile acid transporter inhibitor, an anti-inflammatory agent, an anti-pain medication, an antidepressant, a protein tyrosine phosphatase-1B (PTP-1B) inhibitor, a CB1 antagonist, and a CB1 inverse agonist; and (3) one or more pharmaceutically acceptable carriers.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts).

In one embodiment, the one or more compounds of the invention is administered during at time when the additional therapeutic agent(s) exert their prophylactic or therapeutic effect, or vice versa.

In another embodiment, the one or more compounds of the invention and the additional therapeutic agent(s) are administered in doses commonly employed when such agents are used as monotherapy for treating a condition.

In another embodiment, the one or more compounds of the invention and the additional therapeutic agent(s) are administered in doses lower than the doses commonly employed when such agents are used as monotherapy for treating a condition.

In still another embodiment, the one or more compounds of the invention and the additional therapeutic agent(s) act synergistically and are administered in doses lower than the doses commonly employed when such agents are used as monotherapy for treating a condition.

In one embodiment, the one or more compounds of the invention and the additional therapeutic agent(s) are present in the same composition. In one embodiment, this composition is suitable for oral administration. In another embodiment, this composition is suitable for intravenous administration.

The one or more compounds of the invention and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

In one embodiment, the administration of one or more compounds of the invention and the additional therapeutic agent(s) may inhibit the resistance of a condition to the agent(s).

In one embodiment, when the patient is treated for diabetes, a diabetic complication, impaired glucose tolerance or impaired fasting glucose, the other therapeutic is an antidiabetic agent which is not a compound of the invention. In another embodiment, when the patient is treated for pain, the other therapeutic agent is an analgesic agent which is not a compound of the invention.

In another embodiment, the other therapeutic agent is an agent useful for reducing any potential side effect of a compound of the invention. Non-limiting examples of such potential side effects include nausea, vomiting, headache, fever, lethargy, muscle aches, diarrhea, general pain, and pain at an injection site.

In one embodiment, the other therapeutic agent is used at its known therapeutically effective dose. In another embodiment, the other therapeutic agent is used at its normally prescribed dosage. In another embodiment, the other therapeutic agent is used at less than its normally prescribed dosage or its known therapeutically effective dose.

The doses and dosage regimen of the other agents used in the combination therapies of the present invention for the treatment or prevention of a condition described herein can be determined by the attending clinician, taking into consideration the approved doses and dosage regimen in the package insert; the age, sex and general health of the patient; and the type and severity of the viral infection or related disease or disorder. When administered in combination, the compound(s) of the invention and the other agent(s) for treating diseases or conditions listed above can be administered simultaneously or sequentially. This is particularly useful when the components of the combination are given on different dosing schedules, e.g., one component is administered once daily and another every six hours, or when the preferred pharmaceutical compositions are different, e.g. one is a tablet and one is a capsule. A kit comprising the separate dosage forms is therefore advantageous.

Generally, a total daily dosage of the one or more compounds of the invention and the additional therapeutic agent(s) can, when administered as combination therapy, range from about 0.1 to about 2000 mg per day, although variations will necessarily occur depending on the target of the therapy, the patient and the route of administration. In one embodiment, the dosage is from about 0.2 to about 100 mg/day, administered in a single dose or in 2-4 divided doses. In another embodiment, the dosage is from about 1 to about 500 mg/day, administered in a single dose or in 2-4 divided doses. In another embodiment, the dosage is from about 1 to about 200 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In a further embodiment, the dosage is from about 1 to about 20 mg/day, administered in a single dose or in 2-4 divided doses.

As indicated above, in one embodiment, the invention provides compositions comprising an effective amount of one or more compounds of the invention or a pharmaceutically acceptable salt, solvate, ester or prodrug thereof, and a pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

In one embodiment, the compound of the invention is administered orally.

In another embodiment, the compound of the invention is administered parenterally.

In another embodiment, the compound of the invention is administered intravenously.

In one embodiment, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation is from about 0.1 to about 2000 mg. Variations will necessarily occur depending on the target of the therapy, the patient and the route of administration. In one embodiment, the unit dose dosage is from about 0.2 to about 1000 mg. In another embodiment, the unit dose dosage is from about 1 to about 500 mg. In another embodiment, the unit dose dosage is from about 1 to about 100 mg/day. In still another embodiment, the unit dose dosage is from about 1 to about 50 mg. In yet another embodiment, the unit dose dosage is from about 1 to about 10 mg.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 300 mg/day, preferably 1 mg/day to 75 mg/day, in two to four divided doses.

When the invention comprises a combination of at least one compound of the invention and an additional therapeutic agent, the two active components may be co-administered simultaneously or sequentially, or a single pharmaceutical composition comprising at least one compound of the invention and an additional therapeutic agent in a pharmaceutically acceptable carrier can be administered. The components of the combination can be administered individually or together in any conventional dosage form such as capsule, tablet, powder, cachet, suspension, solution, suppository, nasal spray, etc. The dosage of the additional therapeutic agent can be determined from published material, and may range from about 1 to about 1000 mg per dose. In one embodiment, when used in combination, the dosage levels of the individual components are lower than the recommended individual dosages because of the advantageous effect of the combination.

Thus, the term “pharmaceutical composition” is also intended to encompass both the bulk composition and individual dosage units comprised of more than one (e.g., two) pharmaceutically active agents such as, for example, a compound of the present invention and an additional agent selected from the various the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

In one embodiment, the components of a combination therapy regime are to be administered simultaneously, they can be administered in a single composition with a pharmaceutically acceptable carrier.

In another embodiment, when the components of a combination therapy regime are to be administered separately or sequentially, they can be administered in separate compositions, each containing a pharmaceutically acceptable carrier.

The components of the combination therapy can be administered individually or together in any conventional dosage form such as capsule, tablet, powder, cachet, suspension, solution, suppository, nasal spray, etc.

### Kits

In one embodiment, the present invention provides a kit comprising a effective amount of one or more compounds of the invention, or a pharmaceutically acceptable salt or solvate thereof, and a pharmaceutically acceptable carrier, vehicle or diluent.

In another aspect the present invention provides a kit comprising an amount of one or more compounds of the invention, or a pharmaceutically acceptable salt or solvate thereof, and an amount of at least one additional therapeutic agent described above, wherein the combined amounts are effective for treating or preventing a condition described herein in a patient.

When the components of a combination therapy regime are to are to be administered in more than one composition, they can be provided in a kit comprising in a single package, one container comprising a compound of the invention in pharmaceutically acceptable carrier, and one or more separate containers, each comprising one or more additional therapeutic agents in a pharmaceutically acceptable carrier, with the active components of each composition being present in amounts such that the combination is therapeutically effective.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparant to those skilled in the art and are intended to fall within the scope of the appended claims.

A number of references have been cited herein, the entire disclosures of which are incorporated herein by reference.

1. A compound, or a pharmaceutically acceptable salt, solvate, tautomer, or isomer of said compound, said compound having the general structure shown in Formula (A): 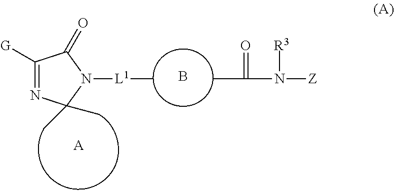
wherein ring A, ring B, L¹, G, R³, and Z are selected independently of each other and wherein:
L¹ is selected from the group consisting of a bond, —N(R⁴)—, —N(R⁴)—(C(R^{5A})₂)—(C(R⁵)₂)_{q}—, —(C(R^{5A})₂)—(C(R⁵)₂)ᵣ(C(R^{5A})₂)—N(R⁴)—, —O—, —O—(C(R^{5A})₂)—(C(R⁵)₂)_{q}—, —(C(R^{5A})₂)—(C(R⁵)₂)ᵣ(C(R^{5A})₂)—O—, and —(C(R^{5A})₂)—(C(R⁵)₂)ₛ—,
each q is independently an integer from 0 to 5;
each r is independently an integer from 0 to 3;
s is an integer from 0 to 5;
ring A represents a spirocycloalkyl ring or a spirocycloalkenyl ring, wherein said ring A is substituted on one or more available ring carbon atoms with from 0 to 5 independently selected R² groups,
or, alternatively, ring A represents a spiroheterocycloalkyl ring or a spiroheterocycloalkenyl ring, wherein said ring A is substituted on one or more available ring carbon atoms with from 0 to 5 independently selected R² groups, and wherein said ring A is optionally further substituted on one or more available ring nitrogen atoms (when present) with from 0 to 3 R^{2A} groups;
ring B is a phenyl ring, wherein said phenyl ring is (in addition to the -L¹- and —C(O)N(R³)—Z moieties shown) optionally further substituted with one or more substituents R^{a}, wherein each R^{a} (when present) is independently selected from the group consisting of halo, —OH, —SF₅, —OSF₅, alkyl, haloalkyl, heteroalkyl, hydroxyalkyl, alkoxy, and —O-haloalkyl,
or ring B is a 5-membered heteroaromatic ring containing from 1 to 3 ring heteroatoms independently selected from N, O, and S, wherein said 5-membered heteroaromatic ring is (in addition to the -L¹- and —C(O)N(R³)—Z moieties shown) optionally further substituted with one or more substituents R^{a}, wherein each R^{a} (when present) is independently selected from the group consisting of halo, —OH, —SF₅, —OSF₅, alkyl, haloalkyl, heteroalkyl, hydroxyalkyl, alkoxy, and —O-haloalkyl,
or ring B is a 6-membered heteroaromatic ring containing from 1 to 3 ring nitrogen atoms, wherein said 6-membered heteroaromatic ring is (in addition to -L¹- and —C(O)N(R³)Z moieties shown) optionally further substituted with one or more substituents R^{a}, wherein each R^{a} (when present) is independently selected from the group consisting of halo, —OH, —SF₅, —OSF₅, alkyl, haloalkyl, hydroxyalkyl, alkoxy, and —O-haloalkyl;
G is independently selected from the group consisting of:
(1) hydrogen, —NH₂, —OH, halo, —SH, —SO₂H, CO₂H, —SF₅, —OSF₅, cyano, —NO₂, —CHO,
(2) cycloalkyl, —O-cycloalkyl, —C(O)-cycloalkyl, —CO₂-cycloalkyl, —S-cycloalkyl, —S(O)-cycloalkyl, —S(O)₂-cycloalkyl, —N(R¹)-cycloalkyl, —C(O)—N(R¹)-cycloalkyl, —N(R¹)—C(O)-cycloalkyl, —N(R¹)—C(O)—N(R¹)-cycloalkyl, —N(R¹)—S(O)-cycloalkyl, —N(R¹)—S(O)₂-cycloalkyl, —N(R¹)—S(O)₂—N(R¹)-cycloalkyl, —S(O)—N(R¹)-cycloalkyl, —S(O)₂—N(R¹)-cycloalkyl,
(3) heterocycloalkyl, —O-heterocycloalkyl, —C(O)-heterocycloalkyl, —CO₂-heterocycloalkyl, —S-heterocycloalkyl, —S(O)-heterocycloalkyl, —S(O)₂-heterocycloalkyl, —N(R¹)-heterocycloalkyl, —C(O)—N(R¹)-heterocycloalkyl, —N(R¹)—C(O)-heterocycloalkyl, —N(R¹)—C(O)—N(R¹)-heterocycloalkyl, —N(R¹)—S(O)-heterocycloalkyl, —N(R¹)—S(O)₂-heterocycloalkyl, —N(R¹)—S(O)₂—N(R¹)-heterocycloalkyl, —S(O)—N(R¹)-heterocycloalkyl, —S(O)₂—N(R¹)-heterocycloalkyl,
(4) cycloalkenyl, —O-cycloalkenyl, —C(O)-cycloalkenyl, —CO₂-cycloalkenyl, —S-cycloalkenyl, —S(O)-cycloalkenyl, —S(O)₂-cycloalkenyl, —N(R¹)-cycloalkenyl, —C(O)—N(R¹)-cycloalkenyl, —N(R¹)—C(O)-cycloalkenyl, —N(R¹)—C(O)—N(R¹)-cycloalkenyl, —N(R¹)—S(O)-cycloalkenyl, —N(R¹)—S(O)₂-cycloalkenyl, —N(R¹)—S(O)₂—N(R¹)-cycloalkenyl, —S(O)—N(R¹)-cycloalkenyl, —S(O)₂—N(R¹)-cycloalkenyl,
(5) heterocycloalkenyl, —O-heterocycloalkenyl, —C(O)-heterocycloalkenyl, —CO₂-heterocycloalkenyl, —S-heterocycloalkenyl, —S(O)-heterocycloalkenyl, —S(O)₂-heterocycloalkenyl, —N(R¹)-heterocycloalkenyl, —C(O)—N(R¹)-heterocycloalkenyl, and —N(R¹)—C(O)-heterocycloalkenyl, —N(R¹)—C(O)—N(R¹)-heterocycloalkenyl, —N(R¹)—S(O)-heterocycloalkenyl, —N(R¹)—S(O)₂-heterocycloalkenyl, —N(R¹)—S(O)₂—N(R¹)-heterocycloalkenyl, —S(O)—N(R¹)-heterocycloalkenyl, —S(O)₂—N(R¹)-heterocycloalkenyl,
(6) alkyl, —O-alkyl, —C(O)-alkyl, —CO₂-alkyl, —S-alkyl, —S(O)-alkyl, —S(O)₂-alkyl, —N(R¹)-alkyl, —C(O)—N(R¹)-alkyl, —N(R¹)—C(O)-alkyl, —N(R¹)—C(O)—N(R¹)-alkyl, —N(R¹)—S(O)-alkyl, —N(R¹)—S(O)₂-alkyl, —N(R¹)—S(O)₂—N(R¹)-alkyl, —S(O)—N(R¹)-alkyl, —S(O)₂—N(R¹)-alkyl,
(7) heteroalkyl, —O-heteroalkyl, —C(O)-heteroalkyl, —CO₂-heteroalkyl, —S-heteroalkyl, —S(O)-heteroalkyl, —S(O)₂-heteroalkyl, —N(R¹)-heteroalkyl, —C(O)—N(R¹)-heteroalkyl, —N(R¹)—C(O)-heteroalkyl, —N(R¹)—C(O)—N(R¹)-heteroalkyl, —N(R¹)—S(O)-heteroalkyl, —N(R¹)—S(O)₂-heteroalkyl, —N(R¹)—S(O)₂—N(R¹)-heteroalkyl, —S(O)—N(R¹)-heteroalkyl, —S(O)₂—N(R¹)-heteroalkyl,
(8) alkenyl, —O-alkenyl, —C(O)-alkenyl, —CO₂-alkenyl, —S-alkenyl, —S(O)-alkenyl, —S(O)₂-alkenyl, —N(R¹)-alkenyl, —C(O)—N(R¹)-alkenyl, —N(R¹)—C(O)-alkenyl, —N(R¹)—C(O)—N(R¹)-alkenyl, —N(R¹)—S(O)-alkenyl, —N(R¹)—S(O)₂-alkenyl, —N(R¹)—S(O)₂—N(R¹)-alkenyl, —S(O)—N(R¹)-alkenyl, —S(O)₂—N(R¹)-alkenyl,
(10) alkynyl, —O-alkynyl, —C(O)-alkynyl, —CO₂-alkynyl, —S-alkynyl, —S(O)-alkynyl, —S(O)₂-alkynyl, —N(R¹)-alkynyl, —C(O)—N(R¹)-alkynyl, —N(R¹)—C(O)-alkynyl, —N(R¹)—C(O)—N(R¹)-alkynyl, —N(R¹)—S(O)-alkynyl, —N(R¹)—S(O)₂-alkynyl, —N(R¹)—S(O)₂—N(R¹)-alkynyl, —S(O)—N(R¹)-alkynyl, and —S(O)₂—N(R¹)-alkynyl;
wherein said heteroalkyl, said heterocycloalkyl, and said heterocycloalkenyl of G may be connected through any available carbon or heteroatom,
and wherein said cycloalkyl, said heterocycloalkyl, said alkenyl, said alkynyl, said cycloalkenyl, and said heterocycloalkenyl of G (when present) are unsubstituted or substituted with one or more groups independently selected from:
(1a)-NH₂, —OH, halo, —SH, —SO₂H, CO₂H, —Si(R⁷)₃, —SF_{S}, —OSF₅, cyano, —NO₂, —CHO,
(2a) cycloalkyl, —O-cycloalkyl, —C(O)-cycloalkyl, —CO₂-cycloalkyl, —S-cycloalkyl, —S(O)-cycloalkyl, —S(O)₂-cycloalkyl, —N(R²⁰)-cycloalkyl, —C(O)—N(R²⁰)-cycloalkyl, —N(R²⁰)—C(O)-cycloalkyl, —N(R²⁰)—C(O)—N(R²⁰)-cycloalkyl, —N(R²⁰)—S(O)-cycloalkyl, —N(R²⁰)—S(O)₂-cycloalkyl, —N(R²⁰)—S(O)₂—N(R²⁰)-cycloalkyl, —S(O)—N(R²⁰)-cycloalkyl, —S(O)₂—N(R²⁰)-cycloalkyl,
(3a) heterocycloalkyl, —O-heterocycloalkyl, —C(O)-heterocycloalkyl, —CO₂-heterocycloalkyl, —S-heterocycloalkyl, —S(O)-heterocycloalkyl, —S(O)₂-heterocycloalkyl, —N(R²⁰)-heterocycloalkyl, —C(O)—N(R²⁰)-heterocycloalkyl, —N(R²⁰)—C(O)-heterocycloalkyl, —N(R²⁰)—C(O)—N(R²⁰)-heterocycloalkyl, —N(R²⁰)—S(O)-heterocycloalkyl, —N(R²⁰)—S(O)₂-heterocycloalkyl, —N(R²⁰)—S(O)₂—N(R²⁰)-heterocycloalkyl, —S(O)—N(R²⁰)-heterocycloalkyl, —S(O)₂—N(R²⁰)-heterocycloalkyl,
(4a) cycloalkenyl, —O-cycloalkenyl, —C(O)-cycloalkenyl, —CO₂-cycloalkenyl, —S-cycloalkenyl, —S(O)-cycloalkenyl, —S(O)₂-cycloalkenyl, —N(R²⁰)-cycloalkenyl, —C(O)—N(R²⁰)-cycloalkenyl, —N(R²⁰)—C(O)-cycloalkenyl, —N(R²⁰)—C(O)—N(R²⁰)-cycloalkenyl, —N(R²⁰)—S(O)-cycloalkenyl, —N(R²⁰)—S(O)₂-cycloalkenyl, —N(R²⁰)—S(O)₂—N(R²⁰)-cycloalkenyl, —S(O)—N(R²⁰)-cycloalkenyl, —S(O)₂—N(R²⁰)-cycloalkenyl,
(5a) heterocycloalkenyl, —O-heterocycloalkenyl, —C(O)-heterocycloalkenyl, —CO₂-heterocycloalkenyl, —S-heterocycloalkenyl, —S(O)-heterocycloalkenyl, —S(O)₂-heterocycloalkenyl, —N(R²⁰)-heterocycloalkenyl, —C(O)—N(R²⁰)-heterocycloalkenyl, and —N(R²⁰)—C(O)-heterocycloalkenyl, —N(R²⁰)—C(O)—N(R²⁰)-heterocycloalkenyl, —N(R²⁰)—S(O)-heterocycloalkenyl, —N(R²⁰)—S(O)₂-heterocycloalkenyl, —N(R²⁰)—S(O)₂—N(R²⁰)-heterocycloalkenyl, —S(O)—N(R²⁰)-heterocycloalkenyl, —S(O)₂—N(R²⁰)-heterocycloalkenyl,
(6a) alkyl, —O-alkyl, —C(O)-alkyl, —CO₂-alkyl, —S-alkyl, —S(O)-alkyl, —S(O)₂-alkyl, —N(R²⁰)-alkyl, —C(O)—N(R²⁰)-alkyl, —N(R²⁰)—C(O)-alkyl, —N(R²⁰)—C(O)—N(R²⁰)-alkyl, —N(R²⁰)—S(O)-alkyl, —N(R²⁰)—S(O)₂-alkyl, —N(R²⁰)—S(O)₂—N(R²⁰)-alkyl, —S(O)—N(R²⁰)-alkyl, —S(O)₂—N(R²⁰)-alkyl,
(7a) heteroalkyl, —O-heteroalkyl, —C(O)-heteroalkyl, —CO₂-heteroalkyl, —S-heteroalkyl, —S(O)-heteroalkyl, —S(O)₂-heteroalkyl, —N(R²⁰)-heteroalkyl, —C(O)—N(R²⁰)-heteroalkyl, —N(R²⁰)—C(O)-heteroalkyl, —N(R²⁰)—C(O)—N(R²⁰)-heteroalkyl, —N(R²⁰)—S(O)-heteroalkyl, —N(R²⁰)—S(O)₂-heteroalkyl, —N(R²⁰)—S(O)₂—N(R²⁰)-heteroalkyl, —S(O)—N(R²⁰)-heteroalkyl, —S(O)₂—N(R²⁰)-heteroalkyl,
(8a) alkenyl, —O-alkenyl, —C(O)-alkenyl, —CO₂-alkenyl, —S-alkenyl, —S(O)-alkenyl, —S(O)₂-alkenyl, —N(R²⁰)-alkenyl, —C(O)—N(R²⁰)-alkenyl, —N(R²⁰)—C(O)-alkenyl, —N(R²⁰)—C(O)—N(R²⁰)-alkenyl, —N(R²⁰)—S(O)-alkenyl, —N(R²⁰)—S(O)₂-alkenyl, —N(R²⁰)—S(O)₂—N(R²⁰)-alkenyl, —S(O)—N(R²⁰)-alkenyl, —S(O)₂—N(R²⁰)-alkenyl,
(10a) alkynyl, —O-alkynyl, —C(O)-alkynyl, —CO₂-alkynyl, —S-alkynyl, —S(O)-alkynyl, —S(O)₂-alkynyl, —N(R²⁰)-alkynyl, —C(O)—N(R²⁰)-alkynyl, —N(R²⁰)—C(O)-alkynyl, —N(R²⁰)—C(O)—N(R²⁰)-alkynyl, —N(R²⁰)—S(O)-alkynyl, —N(R²⁰)—S(O)₂-alkynyl, —N(R²⁰)—S(O)₂—N(R²⁰)-alkynyl, —S(O)—N(R²⁰)-alkynyl, —S(O)₂—N(R²⁰)-alkynyl,
(12a) aryl, —O-aryl, —C(O)-aryl, —CO₂-aryl, —S-aryl, —S(O)-aryl, —S(O)₂-aryl, —N(R²⁰)-aryl, —C(O)—N(R²⁰)-aryl, —N(R²⁰)—C(O)-aryl, —N(R²⁰)—C(O)—N(R²⁰)-aryl, —N(R²⁰)—S(O)-aryl, —N(R²⁰)—S(O)₂-aryl, —N(R²⁰)—S(O)₂—N(R²⁰)-aryl, —S(O)—N(R²⁰)-aryl, —S(O)₂—N(R²⁰)-aryl,
(13a) heteroaryl, —O-heteroaryl, —C(O)-heteroaryl, —CO₂-heteroaryl, —S-heteroaryl, —S(O)-heteroaryl, —S(O)₂-heteroaryl, —N(R²⁰)-heteroaryl, —C(O)—N(R²⁰)-heteroaryl, —N(R²⁰)—C(O)-heteroaryl, —N(R²⁰)—C(O)—N(R²⁰)-heteroaryl, —N(R²⁰)—S(O)-heteroaryl, —N(R²⁰)—S(O)₂-heteroaryl, —N(R²⁰)—S(O)₂—N(R²⁰)-heteroaryl, —S(O)—N(R²⁰)-heteroaryl, —S(O)₂—N(R²⁰)-heteroaryl;
wherein each of said alkyl, said alkenyl, said alkynyl, said cycloalkyl, said cycloalkenyl, said aryl, said heteroalkyl, said heterocycloalkyl, said heterocycloalkenyl, and said heteroaryl of (1a) through (13a) (when present) are each optionally further substituted with one or more groups each independently selected from:
(i) —NH₂, —OH, halo, —SH, —SO₂H, CO₂H, —Si(R⁷)₃, —SF_{S}, —OSF₅, cyano, —NO₂, —CHO,
(ii) cycloalkyl, —O-cycloalkyl, —C(O)-cycloalkyl, —CO₂-cycloalkyl, —S-cycloalkyl, —S(O)-cycloalkyl, —S(O)₂-cycloalkyl, —N(R²⁰)-cycloalkyl, —C(O)—N(R²⁰)-cycloalkyl, —N(R²⁰)—C(O)-cycloalkyl, —N(R²⁰)—C(O)—N(R²⁰)-cycloalkyl, —N(R²⁰)—S(O)-cycloalkyl, —N(R²⁰)—S(O)₂-cycloalkyl, —N(R²⁰)—S(O)₂—N(R²⁰)-cycloalkyl, —S(O)—N(R²⁰)-cycloalkyl, —S(O)₂—N(R²⁰)-cycloalkyl,
(iii) heterocycloalkyl, —O-heterocycloalkyl, —C(O)-heterocycloalkyl, —CO₂-heterocycloalkyl, —S-heterocycloalkyl, —S(O)-heterocycloalkyl, —S(O)₂-heterocycloalkyl, —N(R²⁰)-heterocycloalkyl, —C(O)—N(R²⁰)-heterocycloalkyl, —N(R²⁰)—C(O)-heterocycloalkyl, —N(R²⁰)—C(O)—N(R²⁰)-heterocycloalkyl, —N(R²⁰)—S(O)-heterocycloalkyl, —N(R²⁰)—S(O)₂-heterocycloalkyl, —N(R²⁰)—S(O)₂—N(R²⁰)-heterocycloalkyl, —S(O)—N(R²⁰)-heterocycloalkyl, —S(O)₂—N(R²⁰)-heterocycloalkyl,
(iv) cycloalkenyl, —O-cycloalkenyl, —C(O)-cycloalkenyl, —CO₂-cycloalkenyl, —S-cycloalkenyl, —S(O)-cycloalkenyl, —S(O)₂-cycloalkenyl, —N(R²⁰)-cycloalkenyl, —C(O)—N(R²⁰)-cycloalkenyl, —N(R²⁰)—C(O)-cycloalkenyl, —N(R²⁰)—C(O)—N(R²⁰)-cycloalkenyl, —N(R²⁰)—S(O)-cycloalkenyl, —N(R²⁰)—S(O)₂-cycloalkenyl, —N(R²⁰)—S(O)₂—N(R²⁰)-cycloalkenyl, —S(O)—N(R²⁰)-cycloalkenyl, —S(O)₂—N(R²⁰)-cycloalkenyl,
(v) heterocycloalkenyl, —O-heterocycloalkenyl, —C(O)-heterocycloalkenyl, —CO₂-heterocycloalkenyl, —S-heterocycloalkenyl, —S(O)-heterocycloalkenyl, —S(O)₂-heterocycloalkenyl, —N(R²⁰)-heterocycloalkenyl, —C(O)—N(R²⁰)-heterocycloalkenyl, and —N(R²⁰)—C(O)-heterocycloalkenyl, —N(R²⁰)—C(O)—N(R²⁰)-heterocycloalkenyl, —N(R²⁰)—S(O)-heterocycloalkenyl, —N(R²⁰)—S(O)₂-heterocycloalkenyl, —N(R²⁰)—S(O)₂—N(R²⁰)-heterocycloalkenyl, —S(O)—N(R²⁰)-heterocycloalkenyl, —S(O)₂—N(R²⁰)-heterocycloalkenyl,
(vi) alkyl, —O-alkyl, —C(O)-alkyl, —CO₂-alkyl, —S-alkyl, —S(O)-alkyl, —S(O)₂-alkyl, —N(R²⁰)-alkyl, —C(O)—N(R²⁰)-alkyl, —N(R²⁰)—C(O)-alkyl, —N(R²⁰)—C(O)—N(R²⁰)-alkyl, —N(R²⁰)—S(O)-alkyl, —N(R²⁰)—S(O)₂-alkyl, —N(R²⁰)—S(O)₂—N(R²⁰)-alkyl, —S(O)—N(R²⁰)-alkyl, —S(O)₂—N(R²⁰)-alkyl,
(vii) heteroalkyl, —O-heteroalkyl, —C(O)-heteroalkyl, —CO₂-heteroalkyl, —S-heteroalkyl, —S(O)-heteroalkyl, —S(O)₂-heteroalkyl, —N(R²⁰)-heteroalkyl, —C(O)—N(R²⁰)-heteroalkyl, —N(R²⁰)—C(O)-heteroalkyl, —N(R²⁰)—C(O)—N(R²⁰)-heteroalkyl, —N(R²⁰)—S(O)-heteroalkyl, —N(R²⁰)—S(O)₂-heteroalkyl, —N(R²⁰)—S(O)₂—N(R²⁰)-heteroalkyl, —S(O)—N(R²⁰)-heteroalkyl, —S(O)₂—N(R²⁰)-heteroalkyl,
(viii) alkenyl, —O-alkenyl, —C(O)-alkenyl, —CO₂-alkenyl, —S-alkenyl, —S(O)-alkenyl, —S(O)₂-alkenyl, —N(R²⁰)-alkenyl, —C(O)—N(R²⁰)-alkenyl, —N(R²⁰)—C(O)-alkenyl, —N(R²⁰)—C(O)—N(R²⁰)-alkenyl, —N(R²⁰)—S(O)-alkenyl, —N(R²⁰)—S(O)₂-alkenyl, —N(R²⁰)—S(O)₂—N(R²⁰)-alkenyl, —S(O)—N(R²⁰)-alkenyl, —S(O)₂—N(R²⁰)-alkenyl,
(x) alkynyl, —O-alkynyl, —C(O)-alkynyl, —CO₂-alkynyl, —S-alkynyl, —S(O)-alkynyl, —S(O)₂-alkynyl, —N(R²⁰)-alkynyl, —C(O)—N(R²⁰)-alkynyl, —N(R²⁰)—C(O)-alkynyl, —N(R²⁰)—C(O)—N(R²⁰)-alkynyl, —N(R²⁰)—S(O)-alkynyl, —N(R²⁰)—S(O)₂-alkynyl, —N(R²⁰)—S(O)₂—N(R²⁰)-alkynyl, —S(O)—N(R²⁰)-alkynyl, —S(O)₂—N(R²⁰)-alkynyl,
(xii) aryl, —O-aryl, —C(O)-aryl, —CO₂-aryl, —S-aryl, —S(O)-aryl, —S(O)₂-aryl, —N(R²⁰)-aryl, —C(O)—N(R²⁰)-aryl, —N(R²⁰)—C(O)-aryl, —N(R²⁰)—C(O)—N(R²⁰)-aryl, —N(R²⁰)—S(O)-aryl, —N(R²⁰)—S(O)₂-aryl, —N(R²⁰)—S(O)₂—N(R²⁰)-aryl, —S(O)—N(R²⁰)-aryl, —S(O)₂—N(R²⁰)-aryl,
(xiii) heteroaryl, —O-heteroaryl, —C(O)-heteroaryl, —CO₂-heteroaryl, —S-heteroaryl, —S(O)-heteroaryl, —S(O)₂-heteroaryl, —N(R²⁰)-heteroaryl, —C(O)—N(R²⁰)-heteroaryl, —N(R²⁰)—C(O)-heteroaryl, —N(R²⁰)—C(O)—N(R²⁰)-heteroaryl, —N(R²⁰)—S(O)-heteroaryl, —N(R²⁰)—S(O)₂-heteroaryl, —N(R²⁰)—S(O)₂—N(R²⁰)-heteroaryl, —S(O)—N(R²⁰)-heteroaryl, —S(O)₂—N(R²⁰)-heteroaryl;
and wherein said alkyl and said heteroalkyl of G (when present) are optionally further substituted with one or more groups independently selected from:
(1f) —NH₂, —OH, halo, —SH, —SO₂H, CO₂H, —Si(R⁷)₃, —SF_{S}, —OSF₅, cyano, —NO₂, —CHO,
(2f) cycloalkyl, —O-cycloalkyl, —C(O)-cycloalkyl, —CO₂-cycloalkyl, —S-cycloalkyl, —S(O)-cycloalkyl, —S(O)₂-cycloalkyl, —N(R²⁰)-cycloalkyl, —C(O)—N(R²⁰)-cycloalkyl, —N(R²⁰)—C(O)-cycloalkyl, —N(R²⁰)—C(O)—N(R²⁰)-cycloalkyl, —N(R²⁰)—S(O)-cycloalkyl, —N(R²⁰)—S(O)₂-cycloalkyl, —N(R²⁰)—S(O)₂—N(R²⁰)-cycloalkyl, —S(O)—N(R²⁰)-cycloalkyl, —S(O)₂—N(R²⁰)-cycloalkyl,
(3f) heterocycloalkyl, —O-heterocycloalkyl, —C(O)-heterocycloalkyl, —CO₂-heterocycloalkyl, —S-heterocycloalkyl, —S(O)-heterocycloalkyl, —S(O)₂-heterocycloalkyl, —N(R²⁰)-heterocycloalkyl, —C(O)—N(R²⁰)-heterocycloalkyl, —N(R²⁰)—C(O)-heterocycloalkyl, —N(R²⁰)—C(O)—N(R²⁰)-heterocycloalkyl, —N(R²⁰)—S(O)-heterocycloalkyl, —N(R²⁰)—S(O)₂-heterocycloalkyl, —N(R²⁰)—S(O)₂—N(R²⁰)-heterocycloalkyl, —S(O)—N(R²⁰)-heterocycloalkyl, —S(O)₂—N(R²⁰)-heterocycloalkyl,
(4f) cycloalkenyl, —O-cycloalkenyl, —C(O)-cycloalkenyl, —CO₂-cycloalkenyl, —S-cycloalkenyl, —S(O)-cycloalkenyl, —S(O)₂-cycloalkenyl, —N(R²⁰)-cycloalkenyl, —C(O)—N(R²⁰)-cycloalkenyl, —N(R²⁰)—C(O)-cycloalkenyl, —N(R²⁰)—C(O)—N(R²⁰)-cycloalkenyl, —N(R²⁰)—S(O)-cycloalkenyl, —N(R²⁰)—S(O)₂-cycloalkenyl, —N(R²⁰)—S(O)₂—N(R²⁰)-cycloalkenyl, —S(O)—N(R²⁰)-cycloalkenyl, —S(O)₂—N(R²⁰)-cycloalkenyl,
(5f) heterocycloalkenyl, —O-heterocycloalkenyl, —C(O)-heterocycloalkenyl, —CO₂-heterocycloalkenyl, —S-heterocycloalkenyl, —S(O)-heterocycloalkenyl, —S(O)₂-heterocycloalkenyl, —N(R²⁰)-heterocycloalkenyl, —C(O)—N(R²⁰)-heterocycloalkenyl, and —N(R²⁰)—C(O)-heterocycloalkenyl, —N(R²⁰)—C(O)—N(R²⁰)-heterocycloalkenyl, —N(R²⁰)—S(O)-heterocycloalkenyl, —N(R²⁰)—S(O)₂-heterocycloalkenyl, —N(R²⁰)—S(O)₂—N(R²⁰)-heterocycloalkenyl, —S(O)—N(R²⁰)-heterocycloalkenyl, —S(O)₂—N(R²⁰)-heterocycloalkenyl,
(6f) alkyl, —O-alkyl, —C(O)-alkyl, —CO₂-alkyl, —S-alkyl, —S(O)-alkyl, —S(O)₂-alkyl, —N(R²⁰)-alkyl, —C(O)—N(R²⁰)-alkyl, —N(R²⁰)—C(O)-alkyl, —N(R²⁰)—C(O)—N(R²⁰)-alkyl, —N(R²⁰)—S(O)-alkyl, —N(R²⁰)—S(O)₂-alkyl, —N(R²⁰)—S(O)₂—N(R²⁰)-alkyl, —S(O)—N(R²⁰)-alkyl, —S(O)₂—N(R²⁰)-alkyl,
(7f) heteroalkyl, —O-heteroalkyl, —C(O)-heteroalkyl, —CO₂-heteroalkyl, —S-heteroalkyl, —S(O)-heteroalkyl, —S(O)₂-heteroalkyl, —N(R²⁰)-heteroalkyl, —C(O)—N(R²⁰)-heteroalkyl, —N(R²⁰)—C(O)-heteroalkyl, —N(R²⁰)—C(O)—N(R²⁰)-heteroalkyl, —N(R²⁰)—S(O)-heteroalkyl, —N(R²⁰)—S(O)₂-heteroalkyl, —N(R²⁰)—S(O)₂—N(R²⁰)-heteroalkyl, —S(O)—N(R²⁰)-heteroalkyl, —S(O)₂—N(R²⁰)-heteroalkyl,
(8f) alkenyl, —O-alkenyl, —C(O)-alkenyl, —CO₂-alkenyl, —S-alkenyl, —S(O)-alkenyl, —S(O)₂-alkenyl, —N(R²⁰)-alkenyl, —C(O)—N(R²⁰)-alkenyl, —N(R²⁰)—C(O)-alkenyl, —N(R²⁰)—C(O)—N(R²⁰)-alkenyl, —N(R²⁰)—S(O)-alkenyl, —N(R²⁰)—S(O)₂-alkenyl, —N(R²⁰)—S(O)₂—N(R²⁰)-alkenyl, —S(O)—N(R²⁰)-alkenyl, —S(O)₂—N(R²⁰)-alkenyl,
(10f) alkynyl, —O-alkynyl, —C(O)-alkynyl, —CO₂-alkynyl, —S-alkynyl, —S(O)-alkynyl, —S(O)₂-alkynyl, —N(R²⁰)-alkynyl, —C(O)—N(R²⁰)-alkynyl, —N(R²⁰)—C(O)-alkynyl, —N(R²⁰)—C(O)—N(R²⁰)-alkynyl, —N(R²⁰)—S(O)-alkynyl, —N(R²⁰)—S(O)₂-alkynyl, —N(R²⁰)—S(O)₂—N(R²⁰)-alkynyl, —S(O)—N(R²⁰)-alkynyl, —S(O)₂—N(R²⁰)-alkynyl;
wherein each of said alkyl, said alkenyl, said alkynyl, said cycloalkyl, said cycloalkenyl, said heteroalkyl, said heterocycloalkyl and said heterocycloalkenyl are unsubstituted or optionally independently substituted with one or more groups each independently selected from:
(i) —NH₂, —OH, halo, —SH, —SO₂H, CO₂H, —Si(R⁷)₃, —SF_{S}, —OSF₅, cyano, —NO₂, —CHO,
(ii) cycloalkyl, —O-cycloalkyl, —C(O)-cycloalkyl, —CO₂-cycloalkyl, —S-cycloalkyl, —S(O)-cycloalkyl, —S(O)₂-cycloalkyl, —N(R²⁰)-cycloalkyl, —C(O)—N(R²⁰)-cycloalkyl, —N(R²⁰)—C(O)-cycloalkyl, —N(R²⁰)—C(O)—N(R²⁰)-cycloalkyl, —N(R²⁰)—S(O)-cycloalkyl, —N(R²⁰)—S(O)₂-cycloalkyl, —N(R²⁰)—S(O)₂—N(R²⁰)-cycloalkyl, —S(O)—N(R²⁰)-cycloalkyl, —S(O)₂—N(R²⁰)-cycloalkyl,
(iii) heterocycloalkyl, —O-heterocycloalkyl, —C(O)-heterocycloalkyl, —CO₂-heterocycloalkyl, —S-heterocycloalkyl, —S(O)-heterocycloalkyl, —S(O)₂-heterocycloalkyl, —N(R²⁰)-heterocycloalkyl, —C(O)—N(R²⁰)-heterocycloalkyl, —N(R²⁰)—C(O)-heterocycloalkyl, —N(R²⁰)—C(O)—N(R²⁰)-heterocycloalkyl, —N(R²⁰)—S(O)-heterocycloalkyl, —N(R²⁰)—S(O)₂-heterocycloalkyl, —N(R²⁰)—S(O)₂—N(R²⁰)-heterocycloalkyl, —S(O)—N(R²⁰)-heterocycloalkyl, —S(O)₂—N(R²⁰)-heterocycloalkyl,
(iv) cycloalkenyl, —O-cycloalkenyl, —C(O)-cycloalkenyl, —CO₂-cycloalkenyl, —S-cycloalkenyl, —S(O)-cycloalkenyl, —S(O)₂-cycloalkenyl, —N(R²⁰)-cycloalkenyl, —C(O)—N(R²⁰)-cycloalkenyl, —N(R²⁰)—C(O)-cycloalkenyl, —N(R²⁰)—C(O)—N(R²⁰)-cycloalkenyl, —N(R²⁰)—S(O)-cycloalkenyl, —N(R²⁰)—S(O)₂-cycloalkenyl, —N(R²⁰)—S(O)₂—N(R²⁰)-cycloalkenyl, —S(O)—N(R²⁰)-cycloalkenyl, —S(O)₂—N(R²⁰)-cycloalkenyl,
(v) heterocycloalkenyl, —O-heterocycloalkenyl, —C(O)-heterocycloalkenyl, —CO₂-heterocycloalkenyl, —S-heterocycloalkenyl, —S(O)-heterocycloalkenyl, —S(O)₂-heterocycloalkenyl, —N(R²⁰)-heterocycloalkenyl, —C(O)—N(R²⁰)-heterocycloalkenyl, and —N(R²⁰)—C(O)-heterocycloalkenyl, —N(R²⁰)—C(O)—N(R²⁰)-heterocycloalkenyl, —N(R²⁰)—S(O)-heterocycloalkenyl, —N(R²⁰)—S(O)₂-heterocycloalkenyl, —N(R²⁰)—S(O)₂—N(R²⁰)-heterocycloalkenyl, —S(O)—N(R²⁰)-heterocycloalkenyl, —S(O)₂—N(R²⁰)-heterocycloalkenyl,
(vi) alkyl, —O-alkyl, —C(O)-alkyl, —CO₂-alkyl, —S-alkyl, —S(O)-alkyl, —S(O)₂-alkyl, —N(R²⁰)-alkyl, —C(O)—N(R²⁰)-alkyl, —N(R²⁰)—C(O)-alkyl, —N(R²⁰)—C(O)—N(R²⁰)-alkyl, —N(R²⁰)—S(O)-alkyl, —N(R²⁰)—S(O)₂-alkyl, —N(R²⁰)—S(O)₂—N(R²⁰)-alkyl, —S(O)—N(R²⁰)-alkyl, —S(O)₂—N(R²⁰)-alkyl,
(vii) heteroalkyl, —O-heteroalkyl, —C(O)-heteroalkyl, —CO₂-heteroalkyl, —S-heteroalkyl, —S(O)-heteroalkyl, —S(O)₂-heteroalkyl, —N(R²⁰)-heteroalkyl, —C(O)—N(R²⁰)-heteroalkyl, —N(R²⁰)—C(O)-heteroalkyl, —N(R²⁰)—C(O)—N(R²⁰)-heteroalkyl, —N(R²⁰)—S(O)-heteroalkyl, —N(R²⁰)—S(O)₂-heteroalkyl, —N(R²⁰)—S(O)₂—N(R²⁰)-heteroalkyl, —S(O)—N(R²⁰)-heteroalkyl, —S(O)₂—N(R²⁰)-heteroalkyl,
(viii) alkenyl, —O-alkenyl, —C(O)-alkenyl, —CO₂-alkenyl, —S-alkenyl, —S(O)-alkenyl, —S(O)₂-alkenyl, —N(R²⁰)-alkenyl, —C(O)—N(R²⁰)-alkenyl, —N(R²⁰)—C(O)-alkenyl, —N(R²⁰)—C(O)—N(R²⁰)-alkenyl, —N(R²⁰)—S(O)-alkenyl, —N(R²⁰)—S(O)₂-alkenyl, —N(R²⁰)—S(O)₂—N(R²⁰)-alkenyl, —S(O)—N(R²⁰)-alkenyl, —S(O)₂—N(R²⁰)-alkenyl,
(x) alkynyl, —O-alkynyl, —C(O)-alkynyl, —CO₂-alkynyl, —S-alkynyl, —S(O)-alkynyl, —S(O)₂-alkynyl, —N(R²⁰)-alkynyl, —C(O)—N(R²⁰)-alkynyl, —N(R²⁰)—C(O)-alkynyl, —N(R²⁰)—C(O)—N(R²⁰)-alkynyl, —N(R²⁰)—S(O)-alkynyl, —N(R²⁰)—S(O)₂-alkynyl, —N(R²⁰)—S(O)₂—N(R²⁰)-alkynyl, —S(O)—N(R²⁰)-alkynyl, —S(O)₂—N(R²⁰)-alkynyl,
and wherein said cycloalkyl, said cycloalkenyl, said heterocycloalkyl, and heterocycloalkenyl (when present) of G are optionally unsubstituted or substituted with one or more groups independently selected from: spirocycloalkyl, spirocycloalkenyl, spiroheterocycloalkyl, and spiroheterocycloalkenyl, wherein said spirocycloalkyl, said spirocycloalkenyl, said spiroheterocycloalkyl, and said spiroheterocycloalkenyl are unsubstituted or substituted with one or more groups independently selected from (1a), (2a), (3a), (4a), (5a), (6a), (7a), (8a), (10a), (12a) and (13a) above;
each R¹ is independently selected from:
(1b) hydrogen,
(2b) cycloalkyl, —C(O)-cycloalkyl, —CO₂-cycloalkyl, —S(O)-cycloalkyl, —S(O)₂-cycloalkyl, —C(O)—N(R²⁰)-cycloalkyl, —S(O)—N(R²⁰)-cycloalkyl, —S(O)₂—N(R²⁰)-cycloalkyl,
(3b) heterocycloalkyl, —C(O)-heterocycloalkyl, —CO₂-heterocycloalkyl, —S(O)-heterocycloalkyl, —S(O)₂-heterocycloalkyl, —C(O)—N(R²⁰)-heterocycloalkyl, —S(O)—N(R²⁰)-heterocycloalkyl, —S(O)₂—N(R²⁰)-heterocycloalkyl,
(4b) cycloalkenyl, —C(O)-cycloalkenyl, —CO₂-cycloalkenyl, —S(O)-cycloalkenyl, —S(O)₂-cycloalkenyl, —C(O)—N(R²⁰)-cycloalkenyl, —S(O)—N(R²⁰)-cycloalkenyl, —S(O)₂—N(R²⁰)-cycloalkenyl,
(5b) heterocycloalkenyl, —C(O)-heterocycloalkenyl, —CO₂-heterocycloalkenyl, —S(O)-heterocycloalkenyl, —S(O)₂-heterocycloalkenyl, —C(O)—N(R²⁰)-heterocycloalkenyl, —S(O)—N(R²⁰)-heterocycloalkenyl, —S(O)₂—N(R²⁰)-heterocycloalkenyl,
(6b) alkyl, —C(O)-alkyl, —CO₂-alkyl, —S(O)-alkyl, —S(O)₂-alkyl, —C(O)—N(R²⁰)-alkyl, —S(O)—N(R²⁰)-alkyl, —S(O)₂—N(R²⁰)-alkyl,
(7b) heteroalkyl, —C(O)-heteroalkyl, —CO₂-heteroalkyl, —S(O)-heteroalkyl, —S(O)₂-heteroalkyl, —C(O)—N(R²⁰)-heteroalkyl, —S(O)—N(R²⁰)-heteroalkyl, —S(O)₂—N(R²⁰)-heteroalkyl,
(8b) alkenyl, —C(O)-alkenyl, —CO₂-alkenyl, —S(O)-alkenyl, —S(O)₂-alkenyl, —C(O)—N(R²⁰)-alkenyl, —S(O)—N(R²⁰)-alkenyl, —S(O)₂—N(R²⁰)-alkenyl,
(10b) alkynyl, —C(O)-alkynyl, —CO₂-alkynyl, —S(O)-alkynyl, —S(O)₂-alkynyl, —C(O)—N(R²⁰)-alkynyl, —S(O)—N(R²⁰)-alkynyl, —S(O)₂—N(R²⁰)-alkynyl;
wherein said heteroalkyl, said heterocycloalkyl, and said heterocycloalkenyl of R¹ may be connected through any available carbon or heteroatom,
and wherein said cycloalkyl said heterocycloalkyl, said alkenyl, said alkynyl, said cycloalkenyl, and said heterocycloalkenyl of R¹ are unsubstituted or substituted with one or more groups independently selected from (1a), (2a), (3a), (4a), (5a), (6a), (7a), (8a), (10a), (12a) and (13a) above:
and wherein said alkyl and said heteroalkyl of R¹ are unsubstituted or substituted with one or more groups independently selected from (1f), (2f), (3f), (4f), (5f), (6f), (7f), (8f), and (10f) above;
each R² (when present) is independently selected from the group consisting of:
(1c) —NH₂, —OH, halo, —SH, —SO₂H, CO₂H, —SF_{S}, —OSF₅, cyano, —NO₂, —CHO,
(2c) cycloalkyl, —O-cycloalkyl, —C(O)-cycloalkyl, —CO₂-cycloalkyl, —S-cycloalkyl, —S(O)-cycloalkyl, —S(O)₂-cycloalkyl, —N(R²¹)-cycloalkyl, —C(O)—N(R²¹)-cycloalkyl, —N(R²¹)—C(O)-cycloalkyl, —N(R²¹)—C(O)—N(R²¹)-cycloalkyl, —N(R²¹)—S(O)-cycloalkyl, —N(R²¹)—S(O)₂-cycloalkyl, —N(R²¹)—S(O)₂—N(R²¹)-cycloalkyl, —S(O)—N(R²¹)-cycloalkyl, —S(O)₂—N(R²¹)-cycloalkyl,
(3c) heterocycloalkyl, —O-heterocycloalkyl, —C(O)-heterocycloalkyl, —CO₂-heterocycloalkyl, —S-heterocycloalkyl, —S(O)-heterocycloalkyl, —S(O)₂-heterocycloalkyl, —N(R²¹)-heterocycloalkyl, —C(O)—N(R²¹)-heterocycloalkyl, —N(R²¹)—C(O)-heterocycloalkyl, —N(R²¹)—C(O)—N(R²¹)-heterocycloalkyl, —N(R²¹)—S(O)-heterocycloalkyl, —N(R²¹)—S(O)₂-heterocycloalkyl, —N(R²¹)—S(O)₂—N(R²¹)-heterocycloalkyl, —S(O)—N(R²¹)-heterocycloalkyl, —S(O)₂—N(R²¹)-heterocycloalkyl,
(4c) cycloalkenyl, —O-cycloalkenyl, —C(O)-cycloalkenyl, —CO₂-cycloalkenyl, —S-cycloalkenyl, —S(O)-cycloalkenyl, —S(O)₂-cycloalkenyl, —N(R²¹)-cycloalkenyl, —C(O)—N(R²¹)-cycloalkenyl, —N(R²¹)—C(O)-cycloalkenyl, —N(R²¹)—C(O)—N(R²¹)-cycloalkenyl, —N(R²¹)—S(O)-cycloalkenyl, —N(R²¹)—S(O)₂-cycloalkenyl, —N(R²¹)—S(O)₂—N(R²¹)-cycloalkenyl, —S(O)—N(R²¹)-cycloalkenyl, —S(O)₂—N(R²¹)-cycloalkenyl,
(5c) heterocycloalkenyl, —O-heterocycloalkenyl, —C(O)-heterocycloalkenyl, —CO₂-heterocycloalkenyl, —S-heterocycloalkenyl, —S(O)-heterocycloalkenyl, —S(O)₂-heterocycloalkenyl, —N(R²¹)-heterocycloalkenyl, —C(O)—N(R²¹)-heterocycloalkenyl, and —N(R²¹)—C(O)-heterocycloalkenyl, —N(R²¹)—C(O)—N(R²¹)-heterocycloalkenyl, —N(R²¹)—S(O)-heterocycloalkenyl, —N(R²¹)—S(O)₂-heterocycloalkenyl, —N(R²¹)—S(O)₂—N(R²¹)-heterocycloalkenyl, —S(O)—N(R²¹)-heterocycloalkenyl, —S(O)₂—N(R²¹)-heterocycloalkenyl,
(6c) alkyl, —O-alkyl, —C(O)-alkyl, —CO₂-alkyl, —S-alkyl, —S(O)-alkyl, —S(O)₂-alkyl, —N(R²¹)-alkyl, —C(O)—N(R²¹)-alkyl, —N(R²¹)—C(O)-alkyl, —N(R²¹)—C(O)—N(R²¹)-alkyl, —N(R²¹)—S(O)-alkyl, —N(R²¹)—S(O)₂-alkyl, —N(R²¹)—S(O)₂—N(R²¹)-alkyl, —S(O)—N(R²¹)-alkyl, —S(O)₂—N(R²¹)-alkyl,
(7c) heteroalkyl, —O-heteroalkyl, —C(O)-heteroalkyl, —CO₂-heteroalkyl, —S-heteroalkyl, —S(O)-heteroalkyl, —S(O)₂-heteroalkyl, —N(R²¹)-heteroalkyl, —C(O)—N(R²¹)-heteroalkyl, —N(R²¹)—C(O)-heteroalkyl, —N(R²¹)—C(O)—N(R²¹)-heteroalkyl, —N(R²¹)—S(O)-heteroalkyl, —N(R²¹)—S(O)₂-heteroalkyl, —N(R²¹)—S(O)₂—N(R²¹)-heteroalkyl, —S(O)—N(R²¹)-heteroalkyl, —S(O)₂—N(R²¹)-heteroalkyl,
(8c) alkenyl, —O-alkenyl, —C(O)-alkenyl, —CO₂-alkenyl, —S-alkenyl, —S(O)-alkenyl, —S(O)₂-alkenyl, —N(R²¹)-alkenyl, —C(O)—N(R²¹)-alkenyl, —N(R²¹)—C(O)-alkenyl, —N(R²¹)—C(O)—N(R²¹)-alkenyl, —N(R²¹)—S(O)-alkenyl, —N(R²¹)—S(O)₂-alkenyl, —N(R²¹)—S(O)₂—N(R²¹)-alkenyl, —S(O)—N(R²¹)-alkenyl, —S(O)₂—N(R²¹)-alkenyl,
(10c) alkynyl, —O-alkynyl, —C(O)-alkynyl, —CO₂-alkynyl, —S-alkynyl, —S(O)-alkynyl, —S(O)₂-alkynyl, —N(R²¹)-alkynyl, —C(O)—N(R²¹)-alkynyl, —N(R²¹)—C(O)-alkynyl, —N(R²¹)—C(O)—N(R²¹)-alkynyl, —N(R²¹)—S(O)-alkynyl, —N(R²¹)—S(O)₂-alkynyl, —N(R²¹)—S(O)₂—N(R²¹)-alkynyl, —S(O)—N(R²¹)-alkynyl, —S(O)₂—N(R²¹)-alkynyl,
(12c) aryl, —O-aryl, —C(O)-aryl, —CO₂-aryl, —S-aryl, —S(O)-aryl, —S(O)₂-aryl, —N(R²¹)-aryl, —C(O)—N(R²¹)-aryl, —N(R²¹)—C(O)-aryl, —N(R²¹)—C(O)—N(R²¹)-aryl, —N(R²¹)—S(O)-aryl, —N(R²¹)—S(O)₂-aryl, —N(R²¹)—S(O)₂—N(R²¹)-aryl, —S(O)—N(R²¹)-aryl, —S(O)₂—N(R²¹)-aryl,
(13c) heteroaryl, —O-heteroaryl, —C(O)-heteroaryl, —CO₂-heteroaryl, —S-heteroaryl, —S(O)-heteroaryl, —S(O)₂-heteroaryl, —N(R²¹)-heteroaryl, —C(O)—N(R²¹)-heteroaryl, —N(R²¹)—C(O)-heteroaryl, —N(R²¹)—C(O)—N(R²¹)-heteroaryl, —N(R²¹)—S(O)-heteroaryl, —N(R²¹)—S(O)₂-heteroaryl, —N(R²¹)—S(O)₂—N(R²¹)-heteroaryl, —S(O)—N(R²¹)-heteroaryl, —S(O)₂—N(R²¹)-heteroaryl;
wherein said heteroalkyl, said heterocycloalkyl, said heterocycloalkenyl, and said heteroaryl of R² may be connected through any available carbon or heteroatom,
and wherein said heteroalkyl, said alkyl, said heterocycloalkyl, said cycloalkyl, said alkenyl, said heterocycloalkenyl, said cycloalkenyl, said aryl, said heteroaryl, and said alkynyl of R² are unsubstituted or substituted with one or more groups independently selected from are unsubstituted or substituted with one or more groups independently selected from (1a), (2a), (3a), (4a), (5a), (6a), (7a), (8a), (10a), (12a) and (13a) above;
or, alternatively, two R² groups attached to adjacent ring atoms of ring A are taken together to form a 5-6-membered aromatic or heteroaromatic ring;
or, alternatively, two R² groups attached to the same atom of ring A are taken together to form a moiety selected from the group consisting of carbonyl, spirocycloalkyl, spiroheteroalkyl, spirocycloalkenyl, spiroheterocycloalkenyl, oxime (the oxygen substituents of said oxime being independently selected from R¹⁵), and alkylidene (said alkylidene substituents being independently selected from R¹⁶), wherein said aryl and said heteroaryl of R² are unsubstituted or substituted with one or more groups independently selected from (1a), (2a), (3a), (4a), (5a), (6a), (7a), (8a), (10a), (12a) and (13a) above;
each R^{2A} (when present) is independently selected from the group consisting of:
(1e) cycloalkyl, —C(O)-cycloalkyl, —CO₂-cycloalkyl, —S(O)-cycloalkyl, —S(O)₂-cycloalkyl, —C(O)—N(R²¹)-cycloalkyl, —S(O)—N(R²¹)-cycloalkyl, —S(O)₂—N(R²¹)-cycloalkyl,
(2e) heterocycloalkyl, —C(O)-heterocycloalkyl, —CO₂-heterocycloalkyl, —S(O)-heterocycloalkyl, —S(O)₂-heterocycloalkyl, —C(O)—N(R²¹)-heterocycloalkyl, —S(O)—N(R²¹)-heterocycloalkyl, —S(O)₂—N(R²¹)-heterocycloalkyl,
(3e) cycloalkenyl, —C(O)-cycloalkenyl, —CO₂-cycloalkenyl, —S(O)-cycloalkenyl, —S(O)₂-cycloalkenyl, —C(O)—N(R²¹)-cycloalkenyl, —S(O)—N(R²¹)-cycloalkenyl, —S(O)₂—N(R²¹)-cycloalkenyl,
(4e) heterocycloalkenyl, —C(O)-heterocycloalkenyl, —CO₂-heterocycloalkenyl, —S(O)-heterocycloalkenyl, —S(O)₂-heterocycloalkenyl, —C(O)—N(R²¹)-heterocycloalkenyl, —S(O)—N(R²¹)-heterocycloalkenyl, —S(O)₂—N(R²¹)-heterocycloalkenyl,
(5e) alkyl, —C(O)-alkyl, —CO₂-alkyl, —S(O)-alkyl, —S(O)₂-alkyl, —C(O)—N(R²¹)-alkyl, —S(O)—N(R²¹)-alkyl, —S(O)₂—N(R²¹)-alkyl,
(6e) heteroalkyl, —C(O)-heteroalkyl, —CO₂-heteroalkyl, —S(O)-heteroalkyl, —S(O)₂-heteroalkyl, —C(O)—N(R²¹)-heteroalkyl, —S(O)—N(R²¹)-heteroalkyl, —S(O)₂—N(R²¹)-heteroalkyl,
(7e) alkenyl, —C(O)-alkenyl, —CO₂-alkenyl, —S(O)-alkenyl, —S(O)₂-alkenyl, —C(O)—N(R²¹)-alkenyl, —S(O)—N(R²¹)-alkenyl, —S(O)₂—N(R²¹)-alkenyl,
(9e) alkynyl, —C(O)-alkynyl, —CO₂-alkynyl, —S(O)-alkynyl, —S(O)₂-alkynyl, —C(O)—N(R²¹)-alkynyl, —S(O)—N(R²¹)-alkynyl, —S(O)₂—N(R²¹)-alkynyl,
(11e) aryl, —C(O)-aryl, —CO₂-aryl, —S(O)-aryl, —S(O)₂-aryl, —C(O)—N(R²¹)-aryl, —S(O)—N(R²¹)-aryl, —S(O)₂—N(R²¹)-aryl,
(12e) heteroaryl, —C(O)-heteroaryl, —CO₂-heteroaryl, —S(O)-heteroaryl, —S(O)₂-heteroaryl, —C(O)—N(R²¹)-heteroaryl, —S(O)—N(R²¹)-heteroaryl, —S(O)₂—N(R²¹)-heteroaryl,
(13e) —CHO;
wherein said heteroalkyl, said heterocycloalkyl, said heterocycloalkenyl, and said heteroaryl of R^{2A} may be connected through any available carbon or heteroatom,
and wherein said heteroalkyl, said alkyl, said heterocycloalkyl, said cycloalkyl, said alkenyl, said heterocycloalkenyl, said cycloalkenyl, said aryl, said heteroaryl, and said alkynyl of R^{2A} are unsubstituted or substituted with one or more groups independently selected from are unsubstituted or substituted with one or more groups independently selected from (1a), (2a), (3a), (4a), (5a), (6a), (7a), (8a), (10a), (12a) and (13a) above;
R³ is selected from H and lower alkyl;
Z is a moiety selected from —(C(R¹¹)₂)—(C(R¹²R¹³))ₘ—C(O)OH, —(C(R¹¹)₂)—(C(R¹⁴)₂)ₙ—C(O)OH, from —(C(R¹¹)₂)—(C(R¹²R¹³))ₘ—C(O)Oalkyl, —(C(R¹¹)₂)—(C(R¹⁴)₂)ₙ—C(O)Oalkyl)) 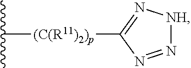
—(C(R¹¹)₂)—(C(R¹²R¹³))ₘ-Q, and —(C(R¹¹)₂)—(C(R¹⁴)₂)ₙ-Q,
wherein Q is a moiety selected from the group consisting of: 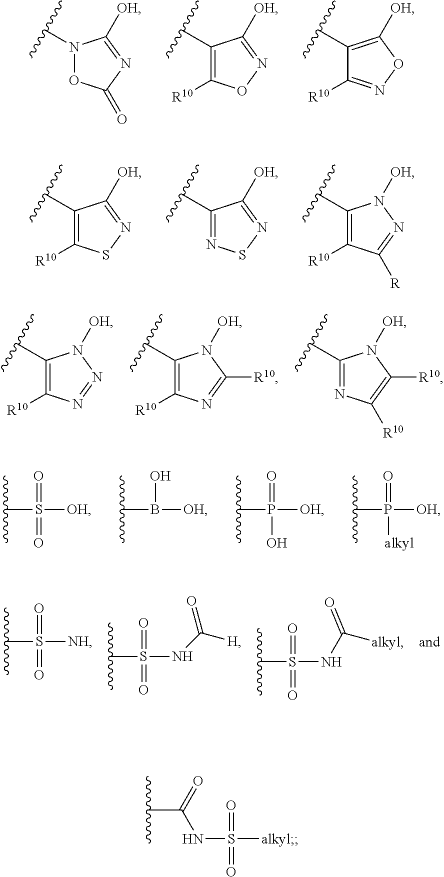
m is an integer from 0 to 5;
n is an integer from 0 to 5;
p is an integer from 0 to 5;
each R⁴ is independently selected from H, —OH, lower alkyl, haloalkyl, alkoxy, heteroalkyl, cyano-substituted lower alkyl, hydroxy-substituted lower alkyl, cycloalkyl, —O-cycloalkyl, —O-alkyl-cycloalkyl, and heterocycloalkyl, —O-heterocycloalkyl, and —O-alkyl-heterocycloalkyl;
each R^{5A} is independently selected from H, alkyl, haloalkyl, heteroalkyl, cyano-substituted alkyl, hydroxy-substituted alkyl, cycloalkyl, -alkyl-cycloalkyl, and heterocycloalkyl, -alkyl-heterocycloalkyl,
or, alternatively, two R^{5A} groups are taken together with the carbon atom to which they are attached to form a carbonyl group, a spirocycloalkyl group, a spiroheterocycloalkyl group, an oxime group, or a substituted oxime group (said oxime substituents being independently selected from alkyl, haloalkyl, hydroxyl-substituted alkyl, and cycloalkyl);
each R⁵ is independently selected from H, —OH, alkyl, haloalkyl, alkoxy, heteroalkyl, cyano-substituted alkyl, hydroxy-substituted alkyl, cycloalkyl, -alkyl-cycloalkyl, —O-cycloalkyl, —O-alkyl-cycloalkyl, and heterocycloalkyl, -alkyl-heterocycloalkyl, —O-heterocycloalkyl, and —O-alkyl-heterocycloalkyl,
or, alternatively, two R⁵ groups bound to the same carbon atom are taken together with the carbon atom to which they are attached to form a carbonyl group, a spirocycloalkyl group, a spiroheterocycloalkyl group, an oxime group, or a substituted oxime group (said oxime substituents being independently selected from alkyl, haloalkyl, hydroxyl-substituted alkyl, and cycloalkyl);
each R⁷ is independently selected from H, alkyl, haloalkyl, heteroalkyl, alkenyl, and alkynyl;
each R¹⁰ is independently selected from H and alkyl;
each R¹¹ is independently selected from H and lower alkyl;
each R¹² is independently selected from H, lower alkyl, —OH, hydroxy-substituted lower alkyl;
each R¹³ is independently selected from H, unsubstituted lower alkyl, lower alkyl substituted with one or more groups each independently selected from hydroxyl and alkoxy, or R¹² and R¹³ are taken together to form an oxo;
each R¹⁴ is independently selected from H and fluoro;
each R¹⁵ is independently selected from H, alkyl, haloalkyl, heteroalkyl, heterocycloalkyl, and cycloalkyl;
each R¹⁶ is independently selected from H, alkyl, haloalkyl, heteroalkyl, heterocycloalkyl, cycloalkyl, aryl, and heteroaryl;
each R²⁰ is independently selected from H, alkyl, haloalkyl, heteroalkyl, alkenyl, and alkynyl;
and each R²¹ is independently selected from:
(1d) hydrogen,
(2d) cycloalkyl, —C(O)-cycloalkyl, —CO₂-cycloalkyl, —S(O)-cycloalkyl, —S(O)₂-cycloalkyl, —C(O)—N(R²⁰)-cycloalkyl, —S(O)—N(R²⁰)-cycloalkyl, —S(O)₂—N(R²⁰)-cycloalkyl,
(3d) heterocycloalkyl, —C(O)-heterocycloalkyl, —CO₂-heterocycloalkyl, —S(O)-heterocycloalkyl, —S(O)₂-heterocycloalkyl, —C(O)—N(R²⁰)-heterocycloalkyl, —S(O)—N(R²⁰)-heterocycloalkyl, —S(O)₂—N(R²⁰)-heterocycloalkyl,
(4d) cycloalkenyl, —C(O)-cycloalkenyl, —CO₂-cycloalkenyl, —S(O)-cycloalkenyl, —S(O)₂-cycloalkenyl, —C(O)—N(R²⁰)-cycloalkenyl, —S(O)—N(R²⁰)-cycloalkenyl, —S(O)₂—N(R²⁰)-cycloalkenyl,
(5d) heterocycloalkenyl, —C(O)-heterocycloalkenyl, —CO₂-heterocycloalkenyl, —S(O)-heterocycloalkenyl, —S(O)₂-heterocycloalkenyl, —C(O)—N(R²⁰)-heterocycloalkenyl, —S(O)—N(R²⁰)-heterocycloalkenyl, —S(O)₂—N(R²⁰)-heterocycloalkenyl,
(6d) alkyl, —C(O)-alkyl, —CO₂-alkyl, —S(O)-alkyl, —S(O)₂-alkyl, —C(O)—N(R²⁰)-alkyl, —S(O)—N(R²⁰)-alkyl, —S(O)₂—N(R²⁰)-alkyl,
(7d) heteroalkyl, —C(O)-heteroalkyl, —CO₂-heteroalkyl, —S(O)-heteroalkyl, —S(O)₂-heteroalkyl, —C(O)—N(R²⁰)-heteroalkyl, —S(O)—N(R²⁰)-heteroalkyl, —S(O)₂—N(R²⁰)-heteroalkyl,
(8d) alkenyl, —C(O)-alkenyl, —CO₂-alkenyl, —S(O)-alkenyl, —S(O)₂-alkenyl, —C(O)—N(R²⁰)-alkenyl, —S(O)—N(R²⁰)-alkenyl, —S(O)₂—N(R²⁰)-alkenyl,
(10d) alkynyl, —C(O)-alkynyl, —CO₂-alkynyl, —S(O)-alkynyl, —S(O)₂-alkynyl, —C(O)—N(R²⁰)-alkynyl, —S(O)—N(R²⁰)-alkynyl, —S(O)₂—N(R²⁰)-alkynyl,
(12d) aryl, —O-aryl, —C(O)-aryl, —CO₂-aryl, —S(O)-aryl, —S(O)₂-aryl, —C(O)—N(R²⁰)-aryl, —S(O)—N(R²⁰)-aryl, —S(O)₂—N(R²⁰)-aryl,
(13d) heteroaryl, —O-heteroaryl, —C(O)-heteroaryl, —CO₂-heteroaryl, —S(O)-heteroaryl, —S(O)₂-heteroaryl, —C(O)—N(R²⁰)-heteroaryl, —S(O)—N(R²⁰)-heteroaryl, —S(O)₂—N(R²⁰)-heteroaryl;
wherein said heteroalkyl, said heterocycloalkyl, said heterocycloalkenyl, and said heteroaryl of R²¹ may be connected through any available carbon or heteroatom,
and wherein said alkyl, said heteroalkyl, said alkenyl, said cycloalkyl, said heterocycloalkyl, said cycloalkenyl, said heterocycloalkenyl, said aryl, said heteroaryl, and said alkynyl of R²¹ are unsubstituted or substituted with one or more groups independently selected from (1a), (2a), (3a), (4a), (5a), (6a), (7a), (8a), (10a), (12a) and (13a) above.

2. A compound of claim 1, or a pharmaceutically acceptable salt, solvate, tautomer, or isomer of said compound, said compound having the general structure shown in Formula (A-1):

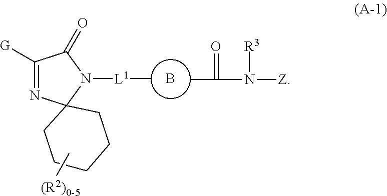

3. A compound of claim 1, or a pharmaceutically acceptable salt, solvate, tautomer, or isomer of said compound, said compound having the general structure shown in Formula (A-1a):

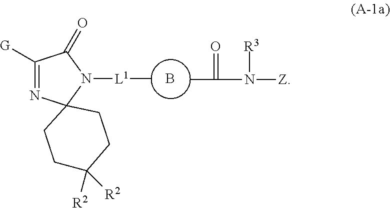

4. A compound of claim 1, or a pharmaceutically acceptable salt, solvate, tautomer, or isomer of said compound, said compound having the general structure shown in Formula (A-1b):

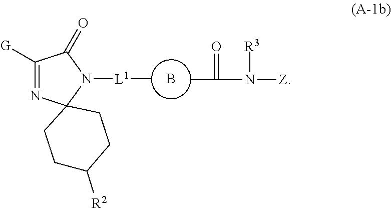

5. A compound according to claim 1, or a pharmaceutically acceptable salt, solvate, tautomer, or isomer of said compound, said compound having the general structure shown in Formula (I): 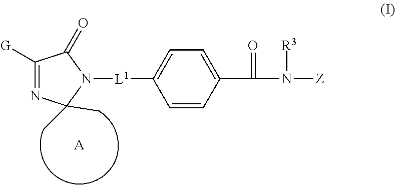
wherein ring A, L¹, G, R³, and Z are selected independently of each other and wherein:
L¹ is selected from the group consisting of: a bond, —N(R⁴)—, —N(R⁴)—(C(R^{5A})₂)—, —O—, —O—(C(R^{5A})₂)—, and —(C(R^{5A})₂)—(C(R⁵)₂)ₛ—;
s is 0-3;
R³ is selected from the group consisting of H and lower alkyl;
Z is a moiety selected from —(C(R¹¹)₂)—(C(R¹²R¹³))ₘ—C(O)OH, —(C(R¹¹)₂)—(C(R¹⁴)₂)ₙ—C(O)OH, and 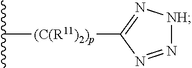
m is an integer from 0 to 5;
n is an integer from 0 to 5;
p is an integer from 0 to 5;
each R⁴ is independently selected from H, lower alkyl, cycloalkyl, heterocycloalkyl, heteroalkyl, and haloalkyl;
each R^{5A} is independently selected from H, lower alkyl, -lower alkyl-Si(CH₃)₃, -lower alkyl-Si(CH₃)₃, lower haloalkyl, and hydroxy-substituted lower alkyl;
each R⁵ is independently selected from H, —OH, lower alkyl, -lower alkyl-Si(CH₃)₃, -lower alkyl-Si(CH₃)₃, lower haloalkyl, and hydroxy-substituted lower alkyl;
each R⁷ is independently selected from H, alkyl, heteroalkyl, and haloalkyl;
each R¹¹ is independently selected from H and lower alkyl;
each R¹² is independently selected from H, lower alkyl, —OH, hydroxy-substituted lower alkyl;
each R¹³ is independently selected from H, unsubstituted lower alkyl, lower alkyl substituted with one or more groups each independently selected from hydroxyl and alkoxy, or R¹² and R¹³ are taken together to form an oxo; and
each R¹⁴ is independently selected from H and fluoro.

6. A compound of claim 5, or a pharmaceutically acceptable salt, solvate, tautomer, or isomer of said compound, wherein:
ring A represents a spirocycloalkyl ring or a spirocycloalkenyl ring, wherein said ring A is substituted on one or more available ring carbon atoms with from 0 to 5 independently selected R² groups;
G is selected from the group consisting of: hydrogen, cycloalkyl, —N(R¹)cycloalkyl, heterocycloalkyl, alkyl, —N(R¹)-alkyl, heteroalkyl, —N(R¹)-heteroalkyl, alkenyl,
wherein said heterocycloalkyl and said heteroalkyl of G may be connected through any available carbon or heteroatom, and wherein said cycloalkyl, said alkenyl and said heterocycloalkyl of G are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
wherein each of said alkyl, said cycloalkyl, and said aryl are unsubstituted or optionally independently substituted with one or more groups each independently selected from: halo, cyano, cycloalkyl, alkyl, —O-alkyl,
and wherein said alkyl and said heteroalkyl of G is unsubstituted or substituted with one or more groups independently selected from: halo, cyano, cycloalkyl, —O-alkyl,
wherein each of said alkyl and said cycloalkyl are unsubstituted or optionally independently substituted with one or more groups each independently selected from: halo, cyano, cycloalkyl, —O-cycloalkyl, alkyl, —O-alkyl,
and wherein R¹ is independently selected from: hydrogen, cycloalkyl, heterocycloalkyl, alkyl, heteroalkyl,
wherein said heteroalkyl and said heterocycloalkyl of R¹ may be connected through any available carbon or heteroatom, and wherein said cycloalkyl and said heterocycloalkyl of R¹ are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
wherein each of said alkyl, said cycloalkyl, and said aryl are unsubstituted or optionally independently substituted with one or more groups each independently selected from:halo, cyano, cycloalkyl, alkyl, —O-alkyl,
and wherein said alkyl and said heteroalkyl of R¹ are unsubstituted or substituted with one or more groups independently selected from: halo, cyano, cycloalkyl, alkyl,
wherein each of said alkyl and said cycloalkyl are unsubstituted or optionally independently substituted with one or more groups each independently selected from: halo, cyano, cycloalkyl, —O-cycloalkyl, alkyl, —O-alkyl,
each R² is independently selected from the group consisting of: halo, —Si(R⁷), cycloalkyl, alkyl;
wherein said alkyl and said cycloalkyl of R² are unsubstituted or substituted with one or more groups independently selected from: halo, —Si(R⁷)₃, —CHO, cycloalkyl, alkyl,
wherein each of said alkyl and cycloalkyl are unsubstituted or optionally independently substituted with one or more groups each independently selected from: halo, —Si(R⁷)₃, —CHO, alkyl.

7. A compound, or a pharmaceutically acceptable salt, solvate, tautomer, or isomer of said compound, said compound having the general structure shown in Formula (II): 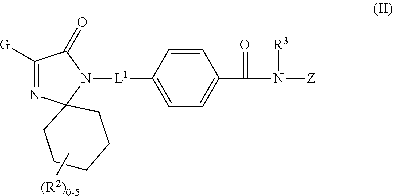
wherein L¹, G, each R², R³, and Z are selected independently of each other and wherein:
L¹ is selected from the group consisting of: a bond and —(C(R^{5A})₂)—(C(R⁵)₂)ₛ—;
s is 0-1;
u is 0 to 2;
v is 1-2;
G is selected from the group consisting of: hydrogen, cycloalkyl, —N(R¹)cycloalkyl, heterocycloalkyl, alkyl, —N(R¹)-alkyl, heteroalkyl, —N(R¹)-heteroalkyl, and alkenyl,
wherein said heterocycloalkyl and said heteroalkyl of G may be connected through any available carbon or heteroatom, and wherein said cycloalkyl, said alkenyl and said heterocycloalkyl of G are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
wherein each of said alkyl, said cycloalkyl, and said aryl are unsubstituted or optionally independently substituted with one or more groups each independently selected from: halo, cyano, cycloalkyl, alkyl, and —O-alkyl,
and wherein said alkyl and said heteroalkyl of G is unsubstituted or substituted with one or more groups independently selected from: halo, cyano, cycloalkyl, —O-alkyl,
wherein each of said alkyl and said cycloalkyl are unsubstituted or optionally independently substituted with one or more groups each independently selected from: halo, cyano, cycloalkyl, —O-cycloalkyl, alkyl, —O-alkyl,
and wherein R¹ is independently selected from: hydrogen, cycloalkyl, heterocycloalkyl, alkyl, heteroalkyl,
wherein said heteroalkyl and said heterocycloalkyl of R¹ may be connected through any available carbon or heteroatom, and wherein said cycloalkyl and said heterocycloalkyl of R¹ are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
wherein each of said alkyl, said cycloalkyl, and said aryl are unsubstituted or optionally independently substituted with one or more groups each independently selected from: halo, cyano, cycloalkyl, alkyl, —O-alkyl,
and wherein said alkyl and said heteroalkyl of R¹ are unsubstituted or substituted with one or more groups independently selected from: halo, cyano, cycloalkyl, alkyl,
wherein each of said alkyl and said cycloalkyl are unsubstituted or optionally independently substituted with one or more groups each independently selected from: halo, cyano, cycloalkyl, —O-cycloalkyl, alkyl, —O-alkyl,
each R² (when present) is independently selected from the group consisting of —Si(CH₃)₃ and alkyl, wherein said alkyl is substituted with from 0 to 5 groups independently selected from: halo, —Si(R⁷)₃, —SF_{S}, —CHO, cycloalkyl, —O-cycloalkyl, —C(O)-cycloalkyl, —CO₂-cycloalkyl, —S(O)-cycloalkyl, —S(O)₂-cycloalkyl, —C(O)—N(R²⁰)-cycloalkyl, —S(O)—N(R²⁰)-cycloalkyl, —S(O)₂—N(R²⁰)-cycloalkyl, —C(O)-heterocycloalkyl, —S(O)₂-heterocycloalkyl, cycloalkenyl, —O-cycloalkenyl, —C(O)-cycloalkenyl, —CO₂-cycloalkenyl, —S(O)-cycloalkenyl, —S(O)₂-cycloalkenyl, —C(O)—N(R²⁰)-cycloalkenyl, —S(O)—N(R²⁰)-cycloalkenyl, —S(O)₂—N(R²⁰)-cycloalkenyl, —C(O)-heterocycloalkenyl, —S(O)₂-heterocycloalkenyl, alkyl, —O-alkyl, —C(O)-alkyl, —CO₂-alkyl, —S(O)-alkyl, —S(O)₂-alkyl, —C(O)—N(R²⁰)-alkyl, —S(O)—N(R²⁰)-alkyl, —S(O)₂—N(R²⁰)-alkyl, —C(O)-heteroalkyl, —S(O)₂-heteroalkyl, alkenyl, —O-alkenyl, —C(O)-alkenyl, —CO₂-alkenyl, —S(O)-alkenyl, —S(O)₂-alkenyl, —C(O)—N(R²⁰)-alkenyl, —S(O)—N(R²⁰)-alkenyl, —S(O)₂—N(R²⁰)-alkenyl, alkynyl, —O-alkynyl, —C(O)-alkynyl, —S(O)-alkynyl, —S(O)₂-alkynyl, —C(O)—N(R²⁰)-alkynyl, —S(O)—N(R²⁰)-alkynyl, —S(O)₂—N(R²⁰)-alkynyl, aryl, —O-aryl, —C(O)-aryl, —CO₂-aryl, —S(O)-aryl, —S(O)₂-aryl, —C(O)—N(R²⁰)-aryl, —S(O)—N(R²⁰)-aryl, —S(O)₂—N(R²⁰)-aryl,
wherein each of said alkyl, said alkenyl, said alkynyl, said cycloalkyl, said cycloalkenyl, said aryl, said heteroalkyl, said heterocycloalkyl, and said heterocycloalkenyl are unsubstituted or optionally independently substituted with one or more groups each independently selected from: —OH, halo, —Si(R⁷)₃, —CHO, cycloalkyl, cycloalkenyl, alkyl, alkenyl, alkynyl, aryl,
R³ is selected from the group consisting of H and lower alkyl;
Z is a moiety selected from the group consisting of: —(CH₂)—(CH(CH₃))—C(O)OH, —(CH₂)—(CF₁₂)—(CF₁₂)—C(O)OH, —(CH₂)—C(CH₃)₂—C(O)OH, —(CH₂)—C(CH₃)(OH)—C(O)OH, —CH₂—CH₂—C(O)OH, —CH₂—CH(OH)—C(O)OH, —CH(CH₃)—CH₂—C(O)OH, —C(CH₃)₂—CH₂—C(O)OH, —CH₂—CH(F)—C(O)OH, —CH₂—CF₂—C(O)OH, —CH(CH₃)—CF₂—C(O)OH, —CH₂—CH₂—CF₂—C(O)OH, and 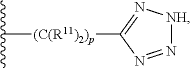
wherein p is an integer from 0 to 1, and R¹¹ (when present) is selected from the group consisting of H and lower alkyl;
each R^{5A} is independently selected from H, lower alkyl, -lower alkyl-Si(CH₃)₃, lower haloalkyl, and lower alkyl substituted with from 1 to 2 hydroxyl;
each R⁵ is independently selected from H, —OH, lower alkyl, -lower alkyl-Si(CH₃)₃, lower haloalkyl, and lower alkyl substituted with from 1 to 2 hydroxyl;
each R⁷ is independently selected from H, alkyl, heteroalkyl, and haloalkyl.

8. A compound of claim 7, or a pharmaceutically acceptable salt, solvate, tautomer, or isomer of said compound, said compound having the general structure shown in Formula (II-a):

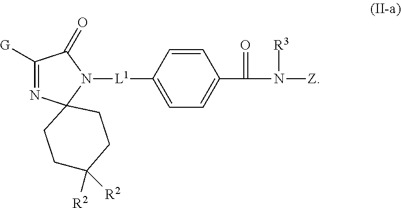

9. A compound of claim 7, or a pharmaceutically acceptable salt, solvate, tautomer, or isomer of said compound, said compound having the general structure shown in Formula (II-b):

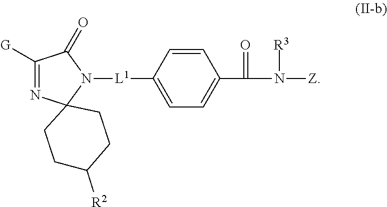

10. A compound of claim 9, or a pharmaceutically acceptable salt, solvate, tautomer, or isomer of said compound, wherein:
L¹ is selected from the group consisting of: a bond, straight or branched lower alkyl, —CH(lower alkyl)-, and —(CH(-lower alkyl-Si(CH₃)₃)—;
G is selected from the group consisting of: hydrogen, cycloalkyl, —N(R¹)cycloalkyl, heterocycloalkyl, alkyl, —N(R¹)-alkyl, heteroalkyl, —N(R¹)-heteroalkyl, alkenyl
wherein said heterocycloalkyl and said heteroalkyl of G may be connected through any available carbon or heteroatom,
and wherein said cycloalkyl, said alkenyl and said heterocycloalkyl of G are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
wherein each of said alkyl, said cycloalkyl, and said aryl are unsubstituted or optionally independently substituted with one or more groups each independently selected from: halo, cyano, cycloalkyl, alkyl, —O-alkyl,
and wherein said alkyl and said heteroalkyl of G is unsubstituted or substituted with one or more groups independently selected from: halo, cyano, cycloalkyl, —O-alkyl,
wherein each of said alkyl and said cycloalkyl are unsubstituted or optionally independently substituted with one or more groups each independently selected from: halo, cyano, cycloalkyl, —O-cycloalkyl, alkyl, —O-alkyl,
and wherein R¹ is independently selected from: hydrogen, cycloalkyl, heterocycloalkyl, alkyl, heteroalkyl,
wherein said heteroalkyl and said heterocycloalkyl of R¹ may be connected through any available carbon or heteroatom,
and wherein said cycloalkyl and said heterocycloalkyl of R¹ are unsubstituted or substituted with one or more groups independently selected from: halo, cyano, cycloalkyl, —O-cycloalkyl, —C(O)-cycloalkyl, alkyl, —O-alkyl, —C(O)-alkyl, aryl,
wherein each of said alkyl, said cycloalkyl, and said aryl are unsubstituted or optionally independently substituted with one or more groups each independently selected from: halo, cyano, cycloalkyl, alkyl, —O-alkyl,
and wherein said alkyl and said heteroalkyl of R¹ are unsubstituted or substituted with one or more groups independently selected from: halo, cyano, cycloalkyl, alkyl,
wherein each of said alkyl and said cycloalkyl are unsubstituted or optionally independently substituted with one or more groups each independently selected from: halo, cyano, cycloalkyl, —O-cycloalkyl, alkyl, —O-alkyl,
each R² is independently selected from the group consisting of H, straight or branched lower alkyl, and —Si(CH₃)₃;
R³ is selected from the group consisting of H and lower alkyl;
Z is a moiety selected from the group consisting of: —(CH₂)—(CH(CH₃))—C(O)OH, —(CH₂)—(CH₂)—(CH₂)—C(O)OH, —(CH₂)—C(CH₃)₂—C(O)OH, —(CH₂)—C(CH₃)(OH)—C(O)OH, —CH₂—CH₂—C(O)OH, —CH₂—CH(OH)—C(O)OH, —CH(CH₃)—CH₂—C(O)OH, —C(CH₃)₂—CH₂—C(O)OH, —(C(R¹¹)₂)—(C(R¹⁴)₂)ₙ—C(O)OH, —CH₂—CH(F)—C(O)OH, —CH₂—CF₂—C(O)OH, —CH(CH₃)—CF₂—C(O)OH, —CH₂—CH₂—CF₂—C(O)OH, —(CH₂)—(CH(CH₃))—C(O)OCH₃, —(CH₂)—(CH₂)—(CH₂)—C(O)OCH₃, —(CH₂)—C(CH₃)₂—C(O)OCH₃, —(CH₂)—C(CH₃)(OH)—C(O)OCH₃, —CH₂—CH₂—C(O)OCH₃, —CH₂—CH(OH)—C(O)OCH₃, —CH(CH₃)—CH₂—C(O)OCH₃, —C(CH₃)₂—CH₂—C(O)OCH₃, —(C(R¹¹)₂)—(C(R¹⁴)₂)ₙ—C(O)OCH₃, —CH₂—CH(F)—C(O)OCH₃, —CH₂—CF₂—C(O)OCH₃, —CH(CH₃)—CF₂—C(O)OCH₃, —CH₂—CH₂—CF₂—C(O)OCH₃, and 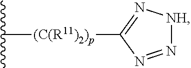
wherein p is an integer from 0 to 1, and R¹¹ (when present) is selected from the group consisting of H and lower alkyl;
each R⁵ is independently selected from H, —OH, lower alkyl, -lower alkyl-Si(CH₃)₃, lower haloalkyl, and lower alkyl substituted with from 1 to 2 hydroxyl; and
each R⁷ is independently selected from H, alkyl, heteroalkyl, and haloalkyl.

11. A compound of claim 10, or a pharmaceutically acceptable salt, solvate, tautomer, or isomer of said compound, wherein:
L¹ is selected from the group consisting of: a bond, 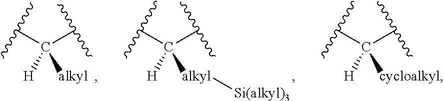
and —(CH₂)₁₋₃—.

12. A compound of claim 10, or a pharmaceutically acceptable salt, solvate, tautomer, or isomer of said compound, wherein:
L¹ is selected from the group consisting of 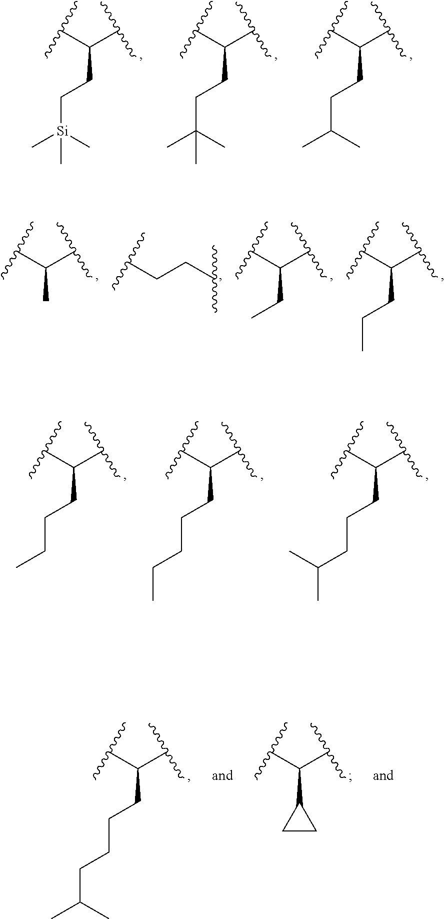
Z is selected from the group consisting of —CH₂—CH₂—C(O)OH and 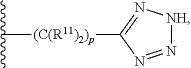
wherein p is 1 and R¹¹ is H.

13. A compound of claim 1, or a pharmaceutically acceptable salt or tautomer of said compound, said compound selected from the group consisting of:

| Ex. | Structure |
|---|---|
| 9.1 | 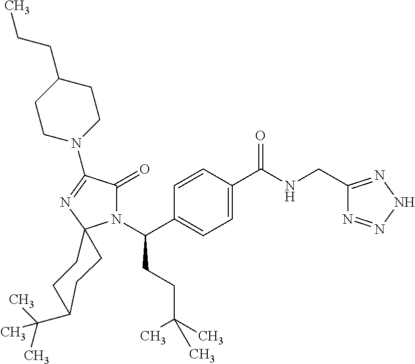 [SMILES: CCCC1CCN(C2=NC3(CCC(C(C)(C)C)CC3)N([C@H](CCC(C)(C)C)c3ccc(C(=O)NCc4nn[nH]n4)cc3)C2=O)CC1] |
| 9.2 | 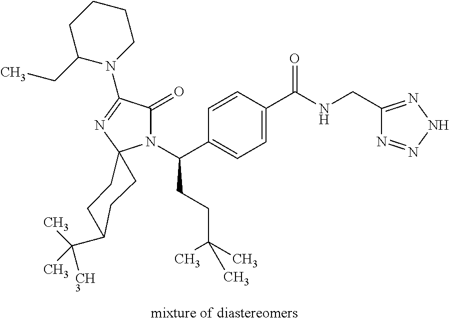 [SMILES: CCC1CCCCN1C1=NC2(CCC(C(C)(C)C)CC2)N([C@H](CCC(C)(C)C)c2ccc(C(=O)NCc3nn[nH]n3)cc2)C1=O] |
| 9.3 | 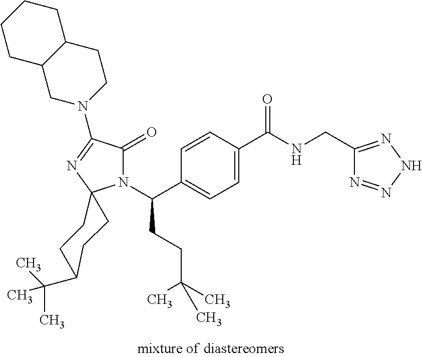 [SMILES: CC(C)(C)CC[C@H](c1ccc(C(=O)NCc2nn[nH]n2)cc1)N1C(=O)C(N2CCC3CCCCC3C2)=NC12CCC(C(C)(C)C)CC2] |
| 9.4 | 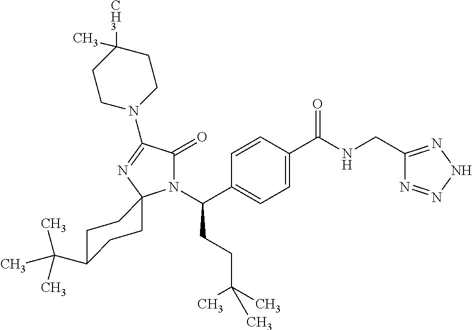 [SMILES: CC(C)(C)CC[C@H](c1ccc(C(=O)NCc2nn[nH]n2)cc1)N1C(=O)C(N2CCC(C)(C)CC2)=NC12CCC(C(C)(C)C)CC2] |
| 9.5 | 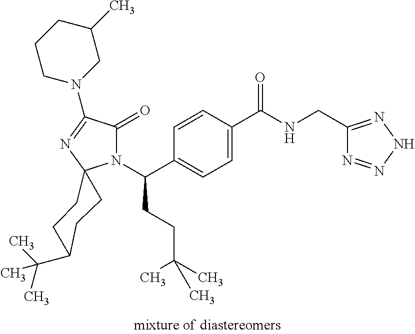 [SMILES: CC1CCCN(C2=NC3(CCC(C(C)(C)C)CC3)N([C@H](CCC(C)(C)C)c3ccc(C(=O)NCc4nn[nH]n4)cc3)C2=O)C1] |
| 9.6 | 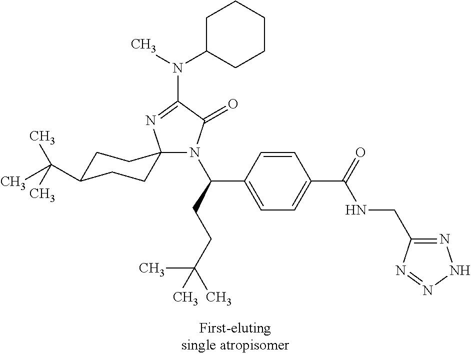 [SMILES: CN(C1=NC2(CCC(C(C)(C)C)CC2)N([C@H](CCC(C)(C)C)c2ccc(C(=O)NCc3nn[nH]n3)cc2)C1=O)C1CCCCC1] |
| 9.7 | 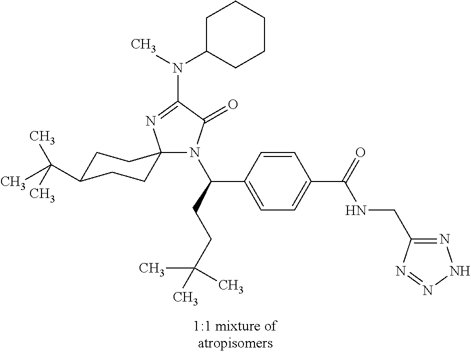 [SMILES: CN(C1=NC2(CCC(C(C)(C)C)CC2)N([C@H](CCC(C)(C)C)c2ccc(C(=O)NCc3nn[nH]n3)cc2)C1=O)C1CCCCC1] |
| 9.8 | 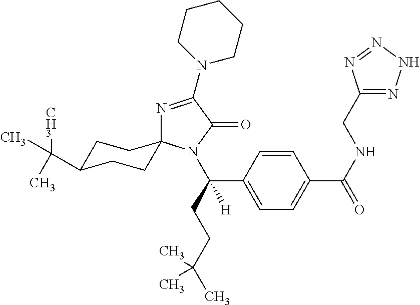 [SMILES: CC(C)(C)CC[C@H](c1ccc(C(=O)NCc2nn[nH]n2)cc1)N1C(=O)C(N2CCCCC2)=NC12CCC(C(C)(C)C)CC2] |
| 9.9 | 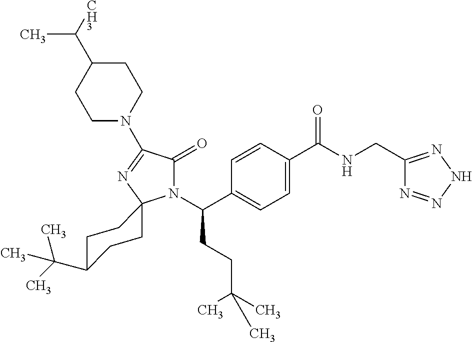 [SMILES: CC(C)C1CCN(C2=NC3(CCC(C(C)(C)C)CC3)N([C@H](CCC(C)(C)C)c3ccc(C(=O)NCc4nn[nH]n4)cc3)C2=O)CC1] |
| 9.10 | 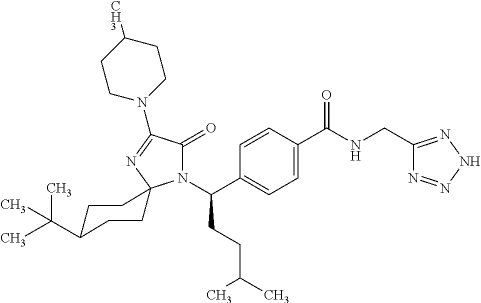 [SMILES: CC(C)CC[C@H](c1ccc(C(=O)NCc2nn[nH]n2)cc1)N1C(=O)C(N2CCC(C)CC2)=NC12CCC(C(C)(C)C)CC2] |
| 9.11 | 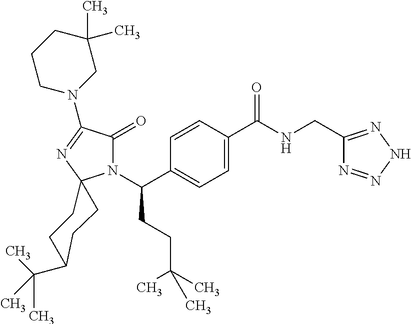 [SMILES: CC(C)(C)CC[C@H](c1ccc(C(=O)NCc2nn[nH]n2)cc1)N1C(=O)C(N2CCCC(C)(C)C2)=NC12CCC(C(C)(C)C)CC2] |
| 9.12 | 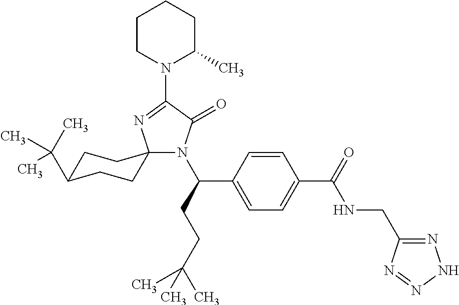 [SMILES: C[C@H]1CCCCN1C1=NC2(CCC(C(C)(C)C)CC2)N([C@H](CCC(C)(C)C)c2ccc(C(=O)NCc3nn[nH]n3)cc2)C1=O] |
| 9.13 | 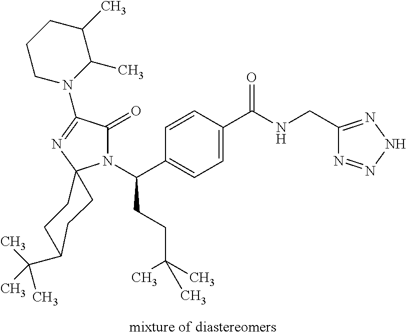 [SMILES: CC1CCCN(C2=NC3(CCC(C(C)(C)C)CC3)N([C@H](CCC(C)(C)C)c3ccc(C(=O)NCc4nn[nH]n4)cc3)C2=O)C1C] |
| 9.14 | 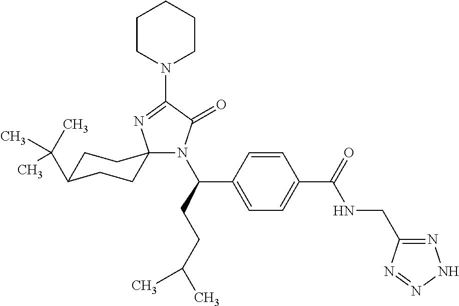 [SMILES: CC(C)CC[C@H](c1ccc(C(=O)NCc2nn[nH]n2)cc1)N1C(=O)C(N2CCCCC2)=NC12CCC(C(C)(C)C)CC2] |
| 9.15 | 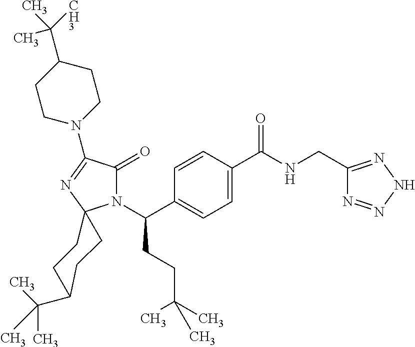 [SMILES: CC(C)(C)CC[C@H](c1ccc(C(=O)NCc2nn[nH]n2)cc1)N1C(=O)C(N2CCC(C(C)(C)C)CC2)=NC12CCC(C(C)(C)C)CC2] |
| 9.16 | 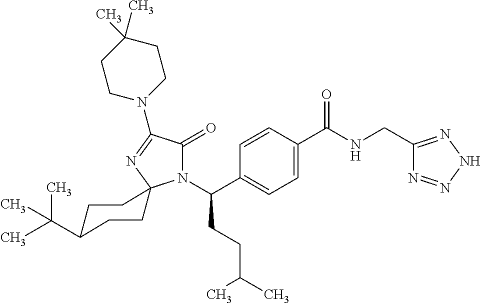 [SMILES: CC(C)CC[C@H](c1ccc(C(=O)NCc2nn[nH]n2)cc1)N1C(=O)C(N2CCC(C)(C)CC2)=NC12CCC(C(C)(C)C)CC2] |
| 9.17 | 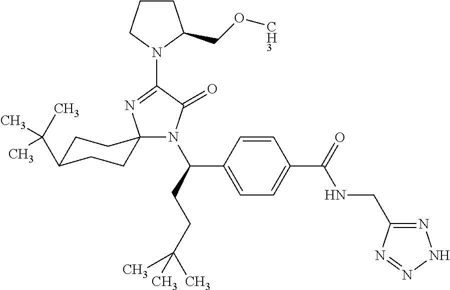 [SMILES: COC[C@@H]1CCCN1C1=NC2(CCC(C(C)(C)C)CC2)N([C@H](CCC(C)(C)C)c2ccc(C(=O)NCc3nn[nH]n3)cc2)C1=O] |
| 9.18 | 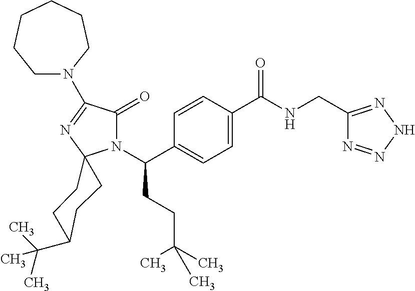 [SMILES: CC(C)(C)CC[C@H](c1ccc(C(=O)NCc2nn[nH]n2)cc1)N1C(=O)C(N2CCCCCC2)=NC12CCC(C(C)(C)C)CC2] |
| 9.19 | 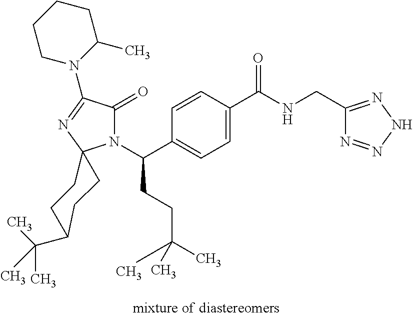 [SMILES: CC1CCCCN1C1=NC2(CCC(C(C)(C)C)CC2)N([C@H](CCC(C)(C)C)c2ccc(C(=O)NCc3nn[nH]n3)cc2)C1=O] |
| 9.20 | 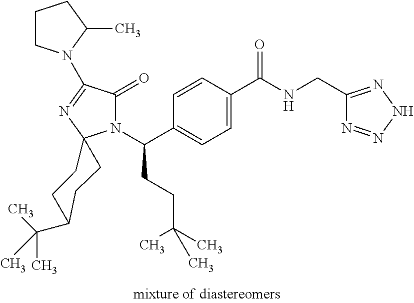 [SMILES: CC1CCCN1C1=NC2(CCC(C(C)(C)C)CC2)N([C@H](CCC(C)(C)C)c2ccc(C(=O)NCc3nn[nH]n3)cc2)C1=O] |
| 9.21 | 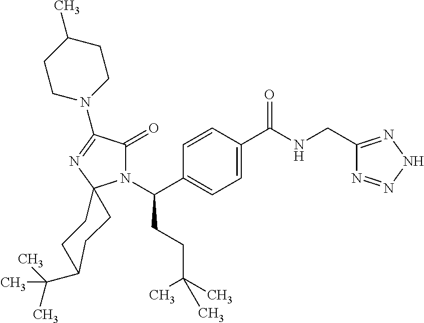 [SMILES: CC1CCN(C2=NC3(CCC(C(C)(C)C)CC3)N([C@H](CCC(C)(C)C)c3ccc(C(=O)NCc4nn[nH]n4)cc3)C2=O)CC1] |
| 9.22 | 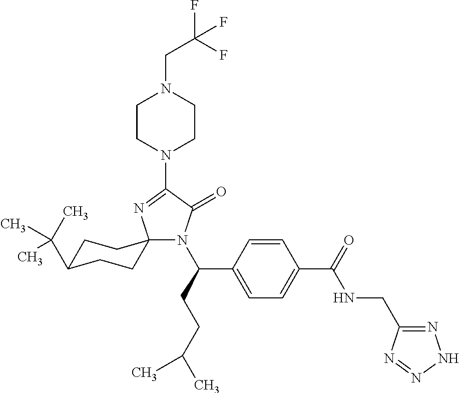 [SMILES: CC(C)CC[C@H](c1ccc(C(=O)NCc2nn[nH]n2)cc1)N1C(=O)C(N2CCN(CC(F)(F)F)CC2)=NC12CCC(C(C)(C)C)CC2] |
| 9.23 | 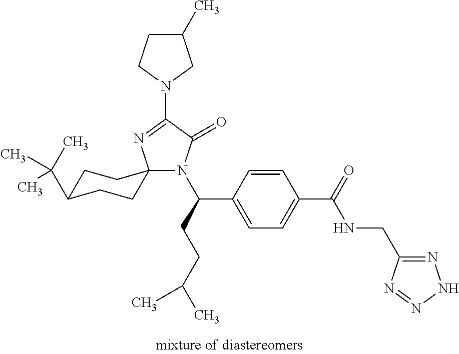 [SMILES: CC(C)CC[C@H](c1ccc(C(=O)NCc2nn[nH]n2)cc1)N1C(=O)C(N2CCC(C)C2)=NC12CCC(C(C)(C)C)CC2] |
| 9.24 | 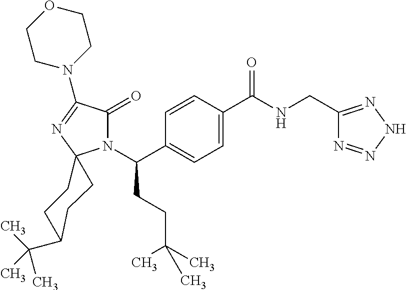 [SMILES: CC(C)(C)CC[C@H](c1ccc(C(=O)NCc2nn[nH]n2)cc1)N1C(=O)C(N2CCOCC2)=NC12CCC(C(C)(C)C)CC2] |
| 9.25 | 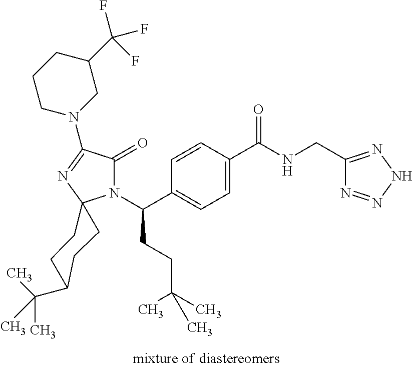 [SMILES: CC(C)(C)CC[C@H](c1ccc(C(=O)NCc2nn[nH]n2)cc1)N1C(=O)C(N2CCCC(C(F)(F)F)C2)=NC12CCC(C(C)(C)C)CC2] |
| 9.26 | 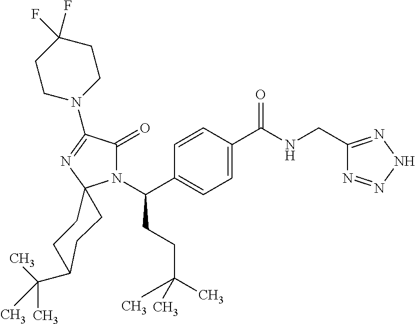 [SMILES: CC(C)(C)CC[C@H](c1ccc(C(=O)NCc2nn[nH]n2)cc1)N1C(=O)C(N2CCC(F)(F)CC2)=NC12CCC(C(C)(C)C)CC2] |
| 9.27 | 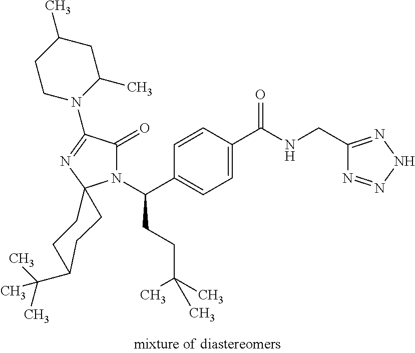 [SMILES: CC1CCN(C2=NC3(CCC(C(C)(C)C)CC3)N([C@H](CCC(C)(C)C)c3ccc(C(=O)NCc4nn[nH]n4)cc3)C2=O)C(C)C1] |
| 9.28 | 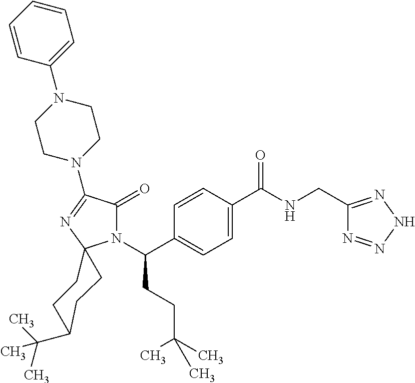 [SMILES: CC(C)(C)CC[C@H](c1ccc(C(=O)NCc2nn[nH]n2)cc1)N1C(=O)C(N2CCN(c3ccccc3)CC2)=NC12CCC(C(C)(C)C)CC2] |
| 9.29 | 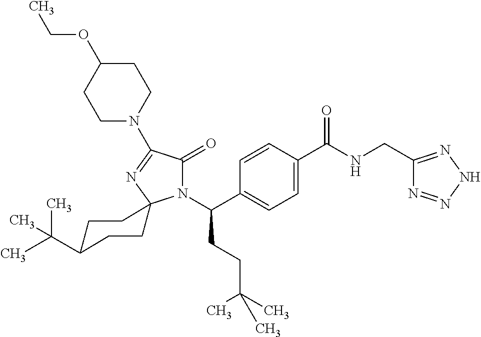 [SMILES: CCOC1CCN(C2=NC3(CCC(C(C)(C)C)CC3)N([C@H](CCC(C)(C)C)c3ccc(C(=O)NCc4nn[nH]n4)cc3)C2=O)CC1] |
| 9.30 | 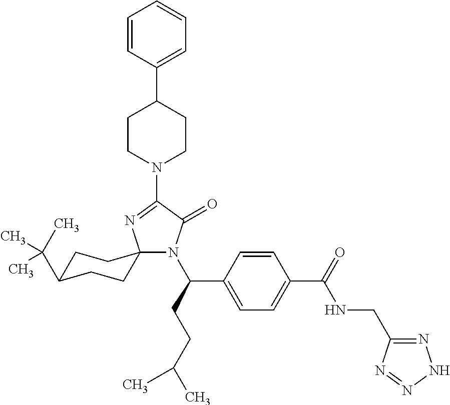 [SMILES: CC(C)CC[C@H](c1ccc(C(=O)NCc2nn[nH]n2)cc1)N1C(=O)C(N2CCC(c3ccccc3)CC2)=NC12CCC(C(C)(C)C)CC2] |
| 9.31 | 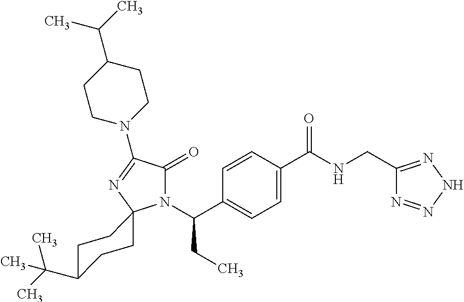 [SMILES: CC[C@H](c1ccc(C(=O)NCc2nn[nH]n2)cc1)N1C(=O)C(N2CCC(C(C)C)CC2)=NC12CCC(C(C)(C)C)CC2] |
| 9.32 | 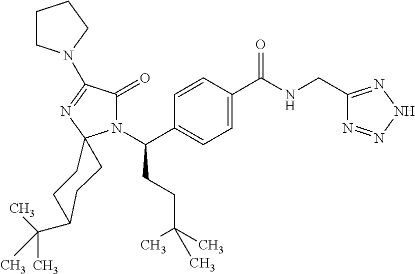 [SMILES: CC(C)(C)CC[C@H](c1ccc(C(=O)NCc2nn[nH]n2)cc1)N1C(=O)C(N2CCCC2)=NC12CCC(C(C)(C)C)CC2] |
| 9.33 | 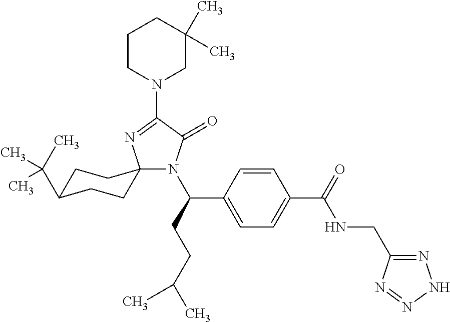 [SMILES: CC(C)CC[C@H](c1ccc(C(=O)NCc2nn[nH]n2)cc1)N1C(=O)C(N2CCCC(C)(C)C2)=NC12CCC(C(C)(C)C)CC2] |
| 9.34 | 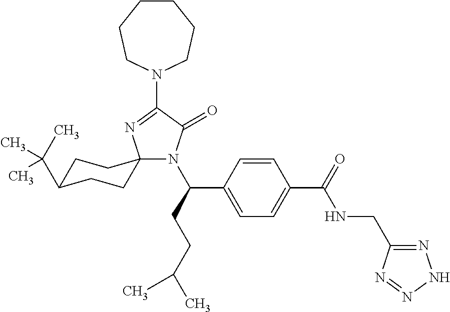 [SMILES: CC(C)CC[C@H](c1ccc(C(=O)NCc2nn[nH]n2)cc1)N1C(=O)C(N2CCCCCC2)=NC12CCC(C(C)(C)C)CC2] |
| 9.35 | 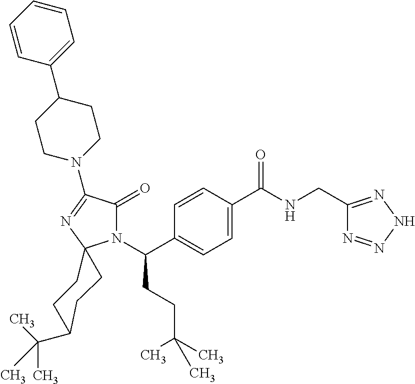 [SMILES: CC(C)(C)CC[C@H](c1ccc(C(=O)NCc2nn[nH]n2)cc1)N1C(=O)C(N2CCC(c3ccccc3)CC2)=NC12CCC(C(C)(C)C)CC2] |
| 9.36 | 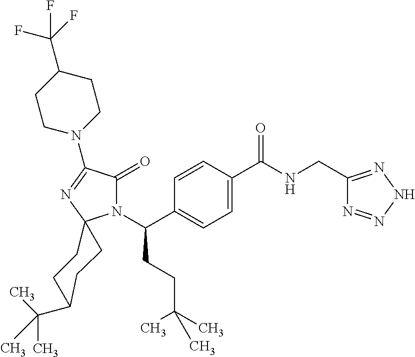 [SMILES: CC(C)(C)CC[C@H](c1ccc(C(=O)NCc2nn[nH]n2)cc1)N1C(=O)C(N2CCC(C(F)(F)F)CC2)=NC12CCC(C(C)(C)C)CC2] |
| 9.37 | 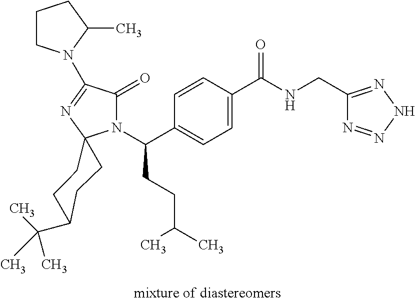 [SMILES: CC(C)CC[C@H](c1ccc(C(=O)NCc2nn[nH]n2)cc1)N1C(=O)C(N2CCCC2C)=NC12CCC(C(C)(C)C)CC2] |
| 9.38 | 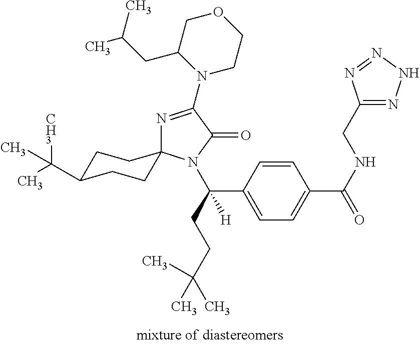 [SMILES: CC(C)CC1COCCN1C1=NC2(CCC(C(C)(C)C)CC2)N([C@H](CCC(C)(C)C)c2ccc(C(=O)NCc3nn[nH]n3)cc2)C1=O] |
| 9.39 | 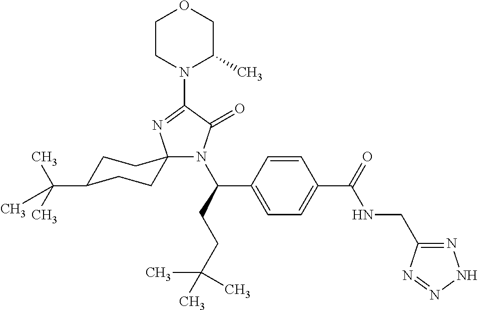 [SMILES: C[C@H]1COCCN1C1=NC2(CCC(C(C)(C)C)CC2)N([C@H](CCC(C)(C)C)c2ccc(C(=O)NCc3nn[nH]n3)cc2)C1=O] |
| 9.40 | 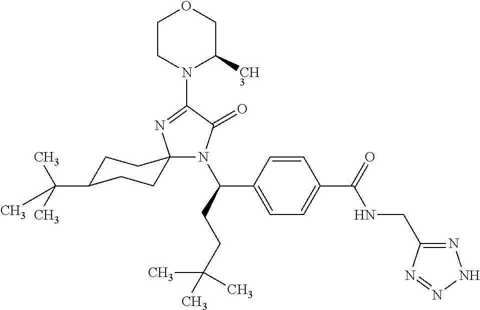 [SMILES: C[C@@H]1COCCN1C1=NC2(CCC(C(C)(C)C)CC2)N([C@H](CCC(C)(C)C)c2ccc(C(=O)NCc3nn[nH]n3)cc2)C1=O] |
| 9.41 | 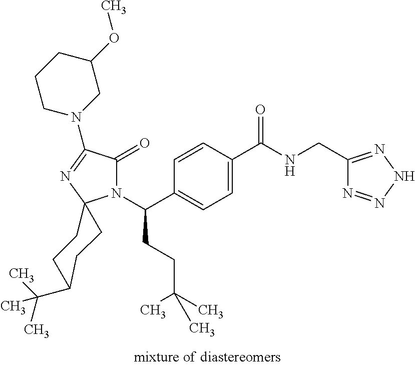 [SMILES: COC1CCCN(C2=NC3(CCC(C(C)(C)C)CC3)N([C@H](CCC(C)(C)C)c3ccc(C(=O)NCc4nn[nH]n4)cc3)C2=O)C1] |
| 9.42 | 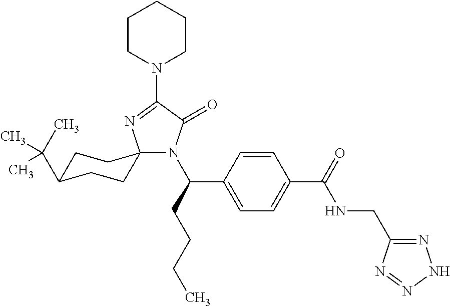 [SMILES: CCCC[C@H](c1ccc(C(=O)NCc2nn[nH]n2)cc1)N1C(=O)C(N2CCCCC2)=NC12CCC(C(C)(C)C)CC2] |
| 9.43 | 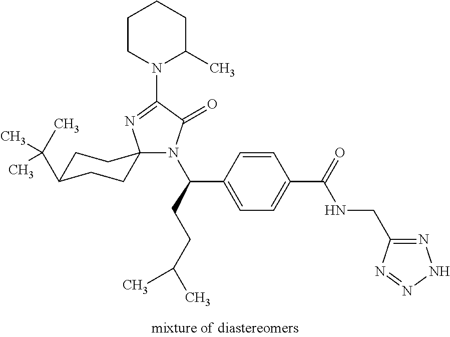 [SMILES: CC(C)CC[C@H](c1ccc(C(=O)NCc2nn[nH]n2)cc1)N1C(=O)C(N2CCCCC2C)=NC12CCC(C(C)(C)C)CC2] |
| 9.44 | 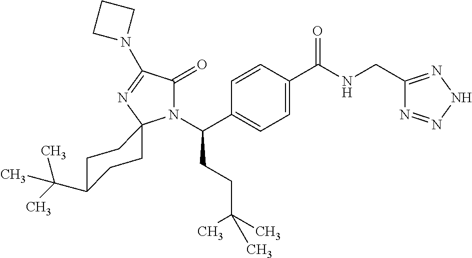 [SMILES: CC(C)(C)CC[C@H](c1ccc(C(=O)NCc2nn[nH]n2)cc1)N1C(=O)C(N2CCC2)=NC12CCC(C(C)(C)C)CC2] |
| 9.45 | 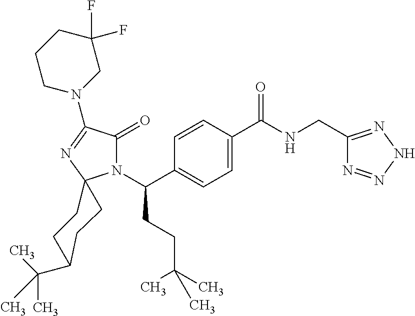 [SMILES: CC(C)(C)CC[C@H](c1ccc(C(=O)NCc2nn[nH]n2)cc1)N1C(=O)C(N2CCCC(F)(F)C2)=NC12CCC(C(C)(C)C)CC2] |
| 9.46 | 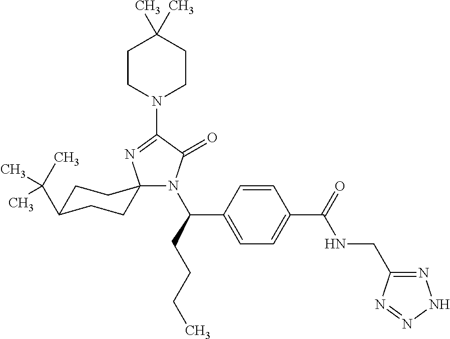 [SMILES: CCCC[C@H](c1ccc(C(=O)NCc2nn[nH]n2)cc1)N1C(=O)C(N2CCC(C)(C)CC2)=NC12CCC(C(C)(C)C)CC2] |
| 9.47 | 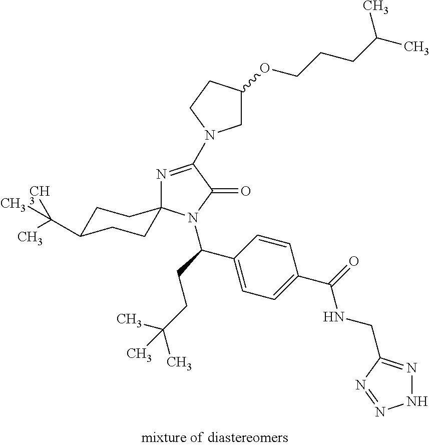 [SMILES: CC(C)CCCOC1CCN(C2=NC3(CCC(C(C)(C)C)CC3)N([C@H](CCC(C)(C)C)c3ccc(C(=O)NCc4nn[nH]n4)cc3)C2=O)C1] |
| 9.48 | 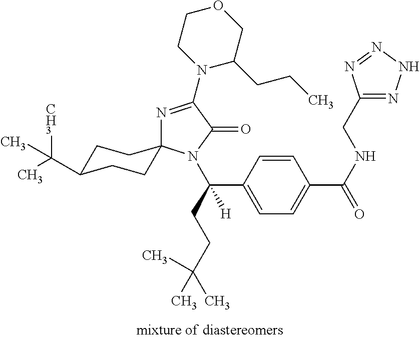 [SMILES: CCCC1COCCN1C1=NC2(CCC(C(C)(C)C)CC2)N([C@H](CCC(C)(C)C)c2ccc(C(=O)NCc3nn[nH]n3)cc2)C1=O] |
| 9.49 | 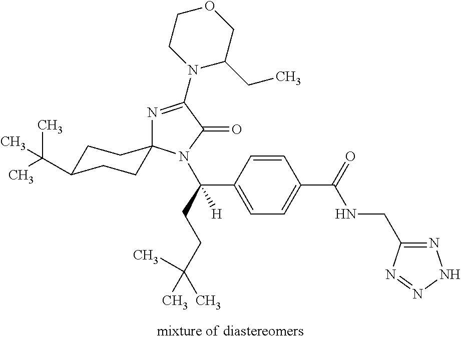 [SMILES: CCC1COCCN1C1=NC2(CCC(C(C)(C)C)CC2)N([C@H](CCC(C)(C)C)c2ccc(C(=O)NCc3nn[nH]n3)cc2)C1=O] |
| 9.50 | 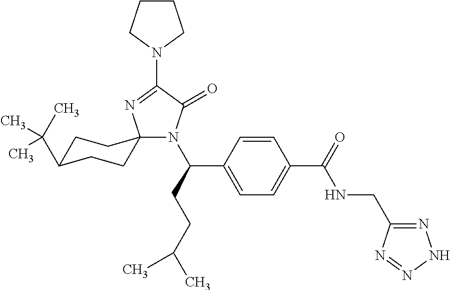 [SMILES: CC(C)CC[C@H](c1ccc(C(=O)NCc2nn[nH]n2)cc1)N1C(=O)C(N2CCCC2)=NC12CCC(C(C)(C)C)CC2] |
| 9.51 | 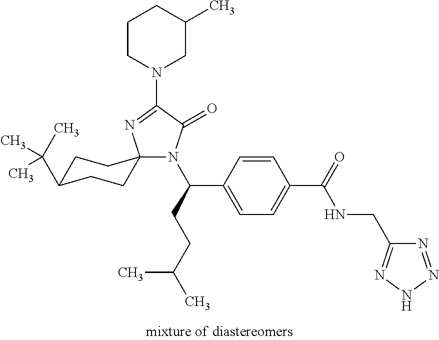 [SMILES: CC(C)CC[C@H](c1ccc(C(=O)NCc2nn[nH]n2)cc1)N1C(=O)C(N2CCCC(C)C2)=NC12CCC(C(C)(C)C)CC2] |
| 9.52 | 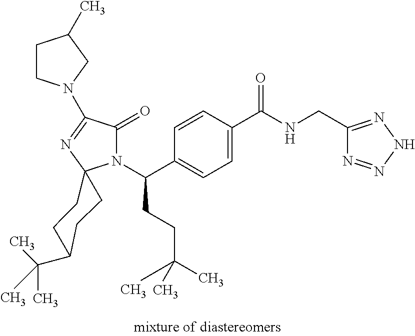 [SMILES: CC1CCN(C2=NC3(CCC(C(C)(C)C)CC3)N([C@H](CCC(C)(C)C)c3ccc(C(=O)NCc4nn[nH]n4)cc3)C2=O)C1] |
| 9.54 | 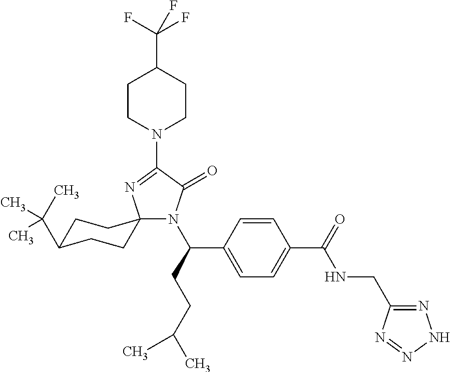 [SMILES: CC(C)CC[C@H](c1ccc(C(=O)NCc2nn[nH]n2)cc1)N1C(=O)C(N2CCC(C(F)(F)F)CC2)=NC12CCC(C(C)(C)C)CC2] |
| 9.55 | 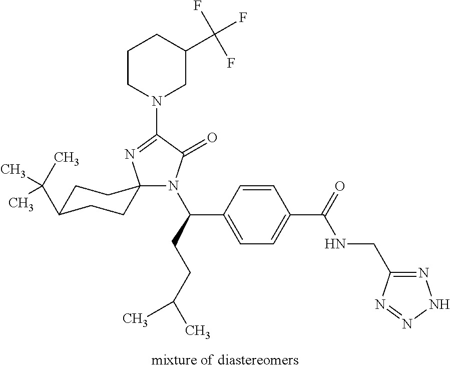 [SMILES: CC(C)CC[C@H](c1ccc(C(=O)NCc2nn[nH]n2)cc1)N1C(=O)C(N2CCCC(C(F)(F)F)C2)=NC12CCC(C(C)(C)C)CC2] |
| 9.56 | 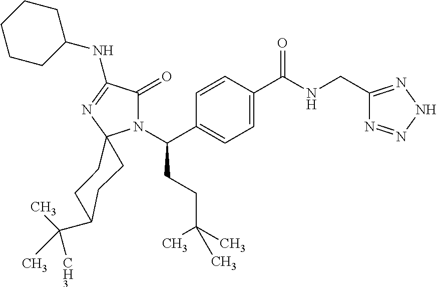 [SMILES: CC(C)(C)CC[C@H](c1ccc(C(=O)NCc2nn[nH]n2)cc1)N1C(=O)C(NC2CCCCC2)=NC12CCC(C(C)(C)C)CC2] |
| 9.57 | 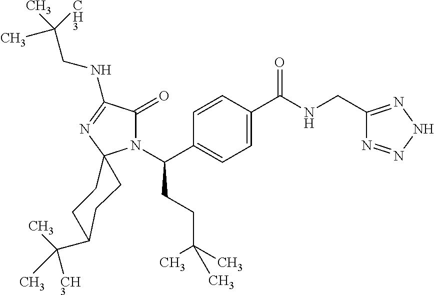 [SMILES: CC(C)(C)CC[C@H](c1ccc(C(=O)NCc2nn[nH]n2)cc1)N1C(=O)C(NCC(C)(C)C)=NC12CCC(C(C)(C)C)CC2] |
| 9.58 | 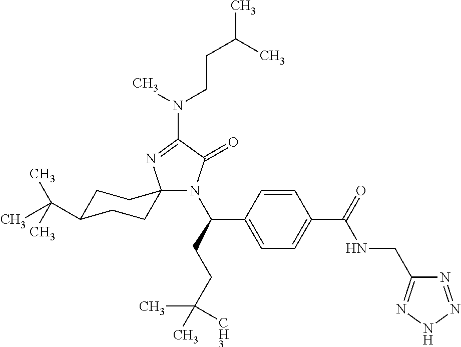 [SMILES: CC(C)CCN(C)C1=NC2(CCC(C(C)(C)C)CC2)N([C@H](CCC(C)(C)C)c2ccc(C(=O)NCc3nn[nH]n3)cc2)C1=O] |
| 9.59 | 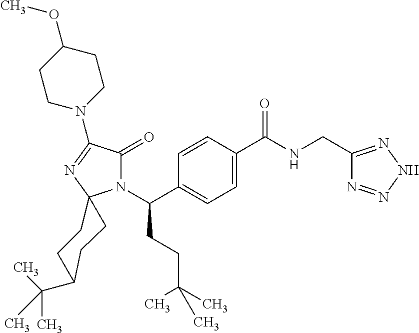 [SMILES: COC1CCN(C2=NC3(CCC(C(C)(C)C)CC3)N([C@H](CCC(C)(C)C)c3ccc(C(=O)NCc4nn[nH]n4)cc3)C2=O)CC1] |
| 9.60 | 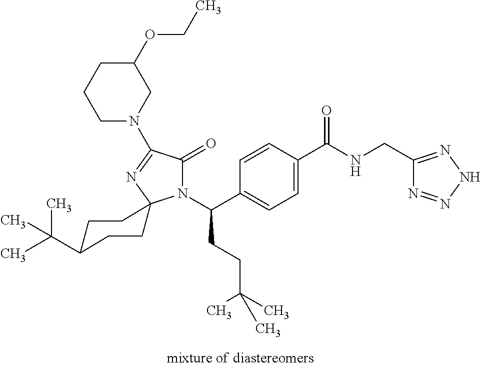 [SMILES: CCOC1CCCN(C2=NC3(CCC(C(C)(C)C)CC3)N([C@H](CCC(C)(C)C)c3ccc(C(=O)NCc4nn[nH]n4)cc3)C2=O)C1] |
| 9.61 | 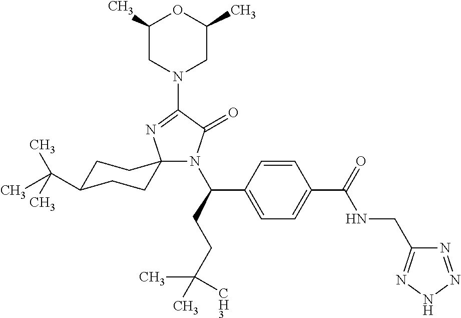 [SMILES: C[C@@H]1CN(C2=NC3(CCC(C(C)(C)C)CC3)N([C@H](CCC(C)(C)C)c3ccc(C(=O)NCc4nn[nH]n4)cc3)C2=O)C[C@H](C)O1] |
| 9.62 | 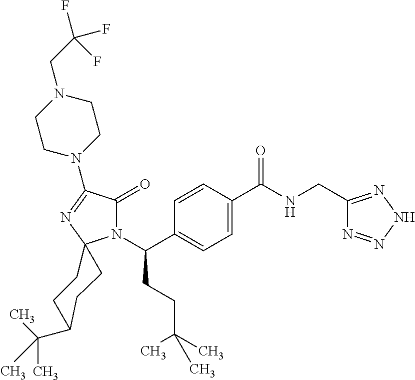 [SMILES: CC(C)(C)CC[C@H](c1ccc(C(=O)NCc2nn[nH]n2)cc1)N1C(=O)C(N2CCN(CC(F)(F)F)CC2)=NC12CCC(C(C)(C)C)CC2] |
| 9.63 | 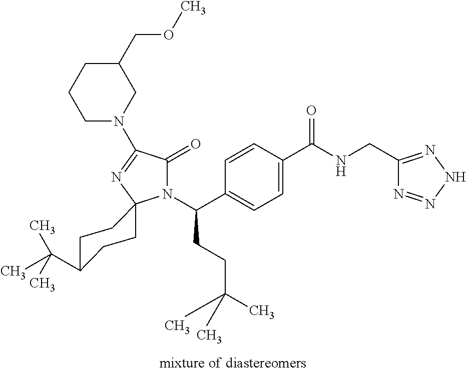 [SMILES: COCC1CCCN(C2=NC3(CCC(C(C)(C)C)CC3)N([C@H](CCC(C)(C)C)c3ccc(C(=O)NCc4nn[nH]n4)cc3)C2=O)C1] |
| 9.64 | 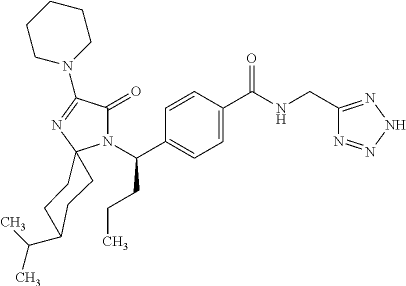 [SMILES: CCC[C@H](c1ccc(C(=O)NCc2nn[nH]n2)cc1)N1C(=O)C(N2CCCCC2)=NC12CCC(C(C)C)CC2] |
| 9.65 | 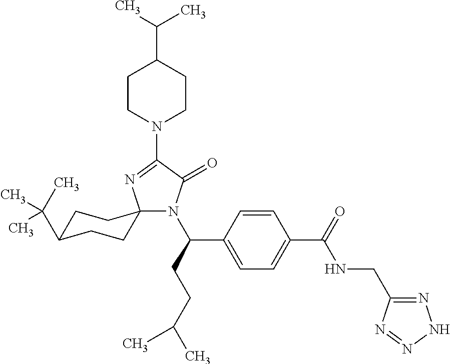 [SMILES: CC(C)CC[C@H](c1ccc(C(=O)NCc2nn[nH]n2)cc1)N1C(=O)C(N2CCC(C(C)C)CC2)=NC12CCC(C(C)(C)C)CC2] |
| 9.66 | 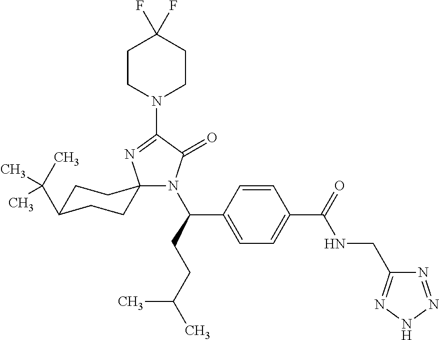 [SMILES: CC(C)CC[C@H](c1ccc(C(=O)NCc2nn[nH]n2)cc1)N1C(=O)C(N2CCC(F)(F)CC2)=NC12CCC(C(C)(C)C)CC2] |
| 9.67 | 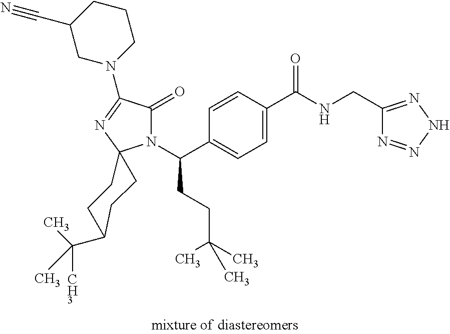 [SMILES: CC(C)(C)CC[C@H](c1ccc(C(=O)NCc2nn[nH]n2)cc1)N1C(=O)C(N2CCCC(C#N)C2)=NC12CCC(C(C)(C)C)CC2] |
| 9.68 | 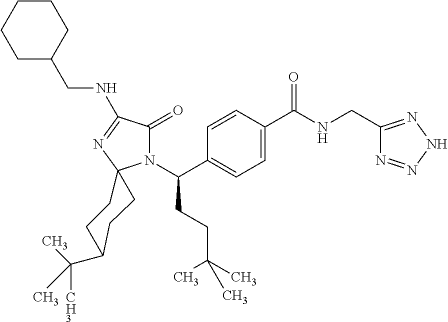 [SMILES: CC(C)(C)CC[C@H](c1ccc(C(=O)NCc2nn[nH]n2)cc1)N1C(=O)C(NCC2CCCCC2)=NC12CCC(C(C)(C)C)CC2] |
| 9.69 | 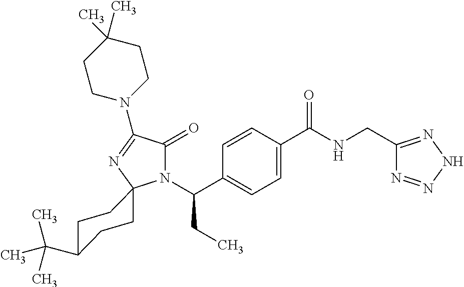 [SMILES: CC[C@H](c1ccc(C(=O)NCc2nn[nH]n2)cc1)N1C(=O)C(N2CCC(C)(C)CC2)=NC12CCC(C(C)(C)C)CC2] |
| 9.70 | 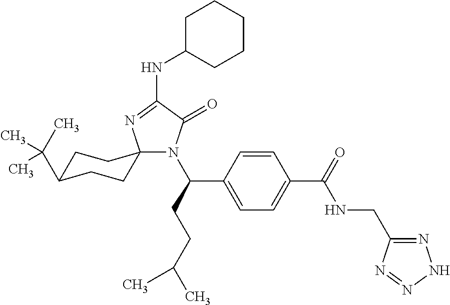 [SMILES: CC(C)CC[C@H](c1ccc(C(=O)NCc2nn[nH]n2)cc1)N1C(=O)C(NC2CCCCC2)=NC12CCC(C(C)(C)C)CC2] |
| 9.71 | 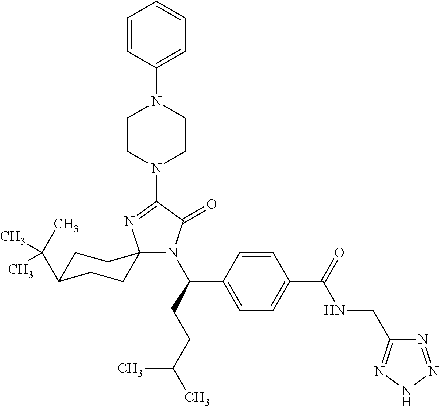 [SMILES: CC(C)CC[C@H](c1ccc(C(=O)NCc2nn[nH]n2)cc1)N1C(=O)C(N2CCN(c3ccccc3)CC2)=NC12CCC(C(C)(C)C)CC2] |
| 9.72 | 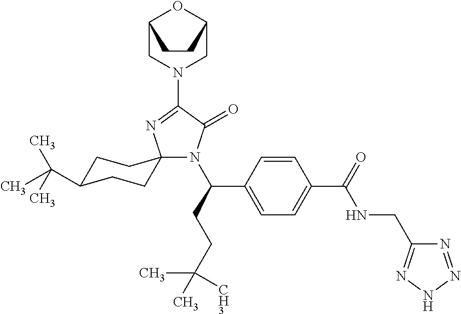 [SMILES: CC(C)(C)CC[C@H](c1ccc(C(=O)NCc2nn[nH]n2)cc1)N1C(=O)C(N2C[C@H]3CC[C@@H](C2)O3)=NC12CCC(C(C)(C)C)CC2] |
| 9.73 | 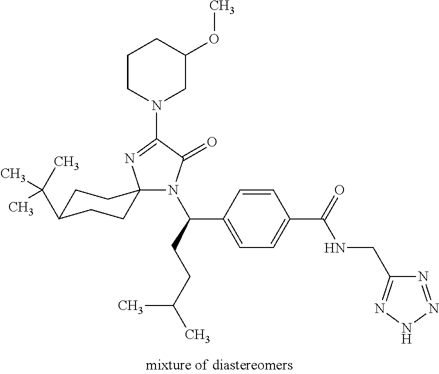 [SMILES: COC1CCCN(C2=NC3(CCC(C(C)(C)C)CC3)N([C@H](CCC(C)C)c3ccc(C(=O)NCc4nn[nH]n4)cc3)C2=O)C1] |
| 9.74 | 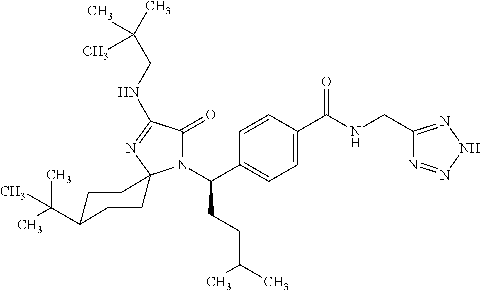 [SMILES: CC(C)CC[C@H](c1ccc(C(=O)NCc2nn[nH]n2)cc1)N1C(=O)C(NCC(C)(C)C)=NC12CCC(C(C)(C)C)CC2] |
| 9.75 | 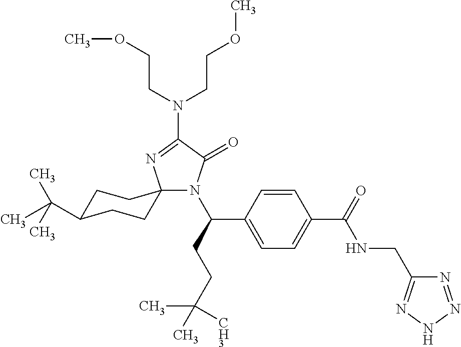 [SMILES: COCCN(CCOC)C1=NC2(CCC(C(C)(C)C)CC2)N([C@H](CCC(C)(C)C)c2ccc(C(=O)NCc3nn[nH]n3)cc2)C1=O] |
| 9.76 | 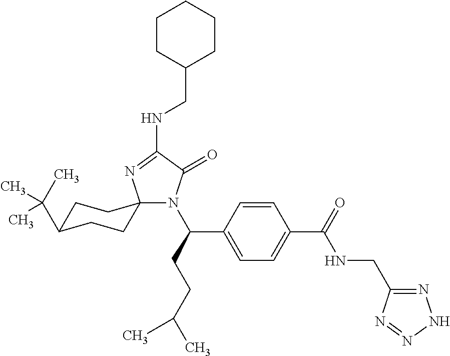 [SMILES: CC(C)CC[C@H](c1ccc(C(=O)NCc2nn[nH]n2)cc1)N1C(=O)C(NCC2CCCCC2)=NC12CCC(C(C)(C)C)CC2] |
| 9.77 | 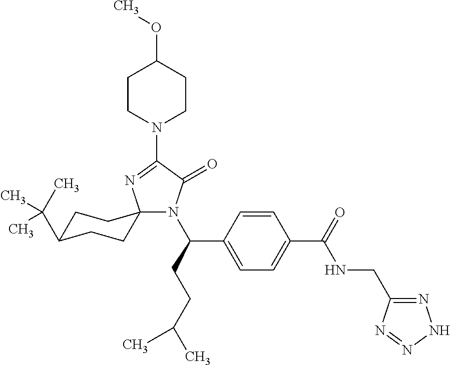 [SMILES: COC1CCN(C2=NC3(CCC(C(C)(C)C)CC3)N([C@H](CCC(C)C)c3ccc(C(=O)NCc4nn[nH]n4)cc3)C2=O)CC1] |
| 9.78 | 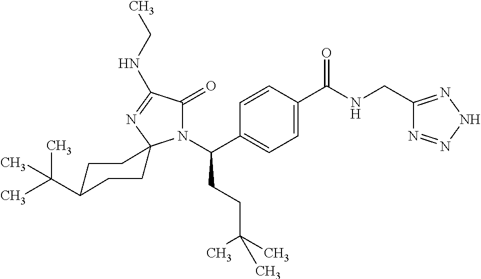 [SMILES: CCNC1=NC2(CCC(C(C)(C)C)CC2)N([C@H](CCC(C)(C)C)c2ccc(C(=O)NCc3nn[nH]n3)cc2)C1=O] |
| 9.79 | 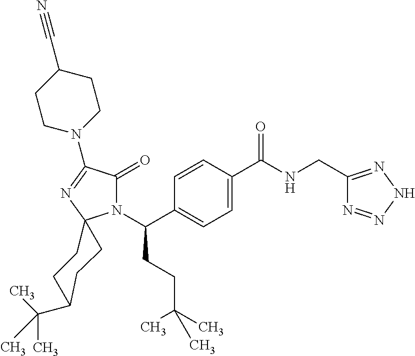 [SMILES: CC(C)(C)CC[C@H](c1ccc(C(=O)NCc2nn[nH]n2)cc1)N1C(=O)C(N2CCC(C#N)CC2)=NC12CCC(C(C)(C)C)CC2] |
| 9.81 | 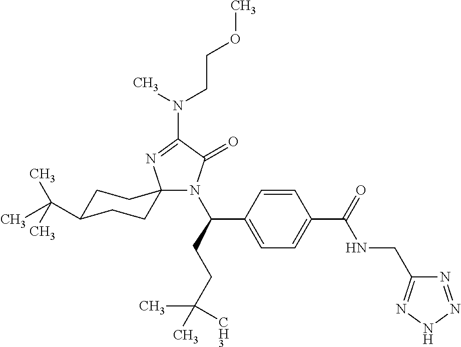 [SMILES: COCCN(C)C1=NC2(CCC(C(C)(C)C)CC2)N([C@H](CCC(C)(C)C)c2ccc(C(=O)NCc3nn[nH]n3)cc2)C1=O] |
| 9.82 | 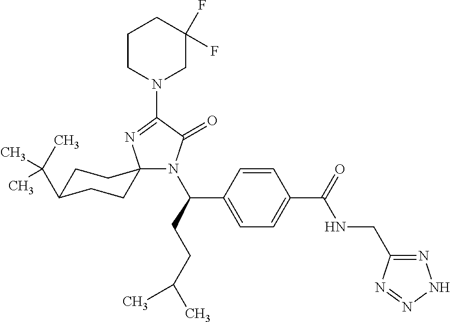 [SMILES: CC(C)CC[C@H](c1ccc(C(=O)NCc2nn[nH]n2)cc1)N1C(=O)C(N2CCCC(F)(F)C2)=NC12CCC(C(C)(C)C)CC2] |
| 9.83 | 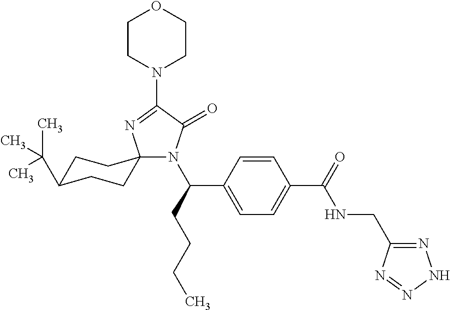 [SMILES: CCCC[C@H](c1ccc(C(=O)NCc2nn[nH]n2)cc1)N1C(=O)C(N2CCOCC2)=NC12CCC(C(C)(C)C)CC2] |
| 9.84 | 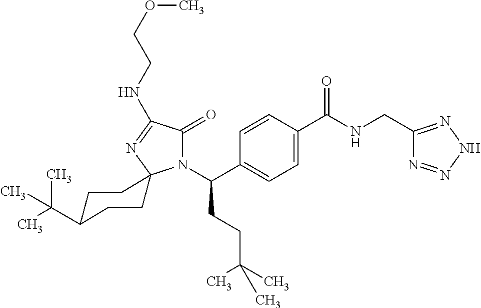 [SMILES: COCCNC1=NC2(CCC(C(C)(C)C)CC2)N([C@H](CCC(C)(C)C)c2ccc(C(=O)NCc3nn[nH]n3)cc2)C1=O] |
| 9.86 | 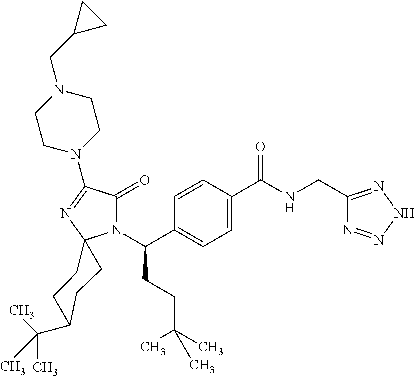 [SMILES: CC(C)(C)CC[C@H](c1ccc(C(=O)NCc2nn[nH]n2)cc1)N1C(=O)C(N2CCN(CC3CC3)CC2)=NC12CCC(C(C)(C)C)CC2] |
| 9.87 | 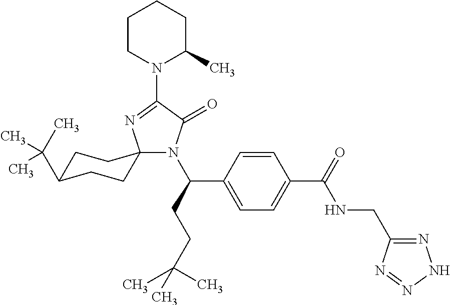 [SMILES: C[C@@H]1CCCCN1C1=NC2(CCC(C(C)(C)C)CC2)N([C@H](CCC(C)(C)C)c2ccc(C(=O)NCc3nn[nH]n3)cc2)C1=O] |
| 9.88 | 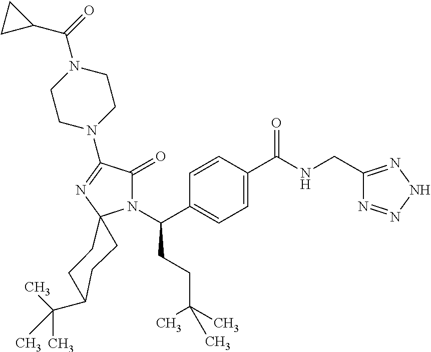 [SMILES: CC(C)(C)CC[C@H](c1ccc(C(=O)NCc2nn[nH]n2)cc1)N1C(=O)C(N2CCN(C(=O)C3CC3)CC2)=NC12CCC(C(C)(C)C)CC2] |
| 9.89 | 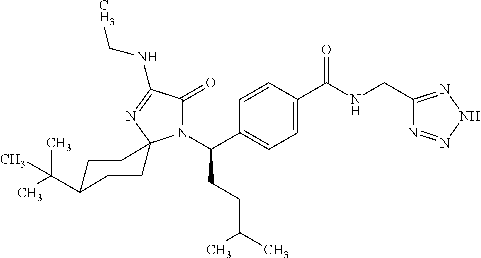 [SMILES: CCNC1=NC2(CCC(C(C)(C)C)CC2)N([C@H](CCC(C)C)c2ccc(C(=O)NCc3nn[nH]n3)cc2)C1=O] |
| 9.90 | 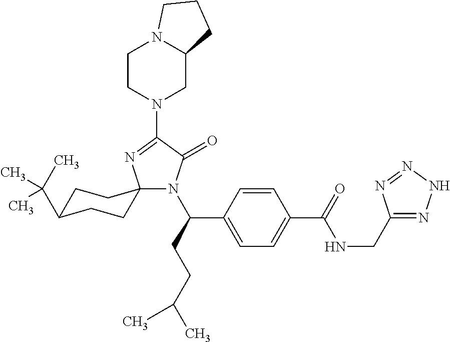 [SMILES: CC(C)CC[C@H](c1ccc(C(=O)NCc2nn[nH]n2)cc1)N1C(=O)C(N2CCN3CCC[C@H]3C2)=NC12CCC(C(C)(C)C)CC2] |
| 9.91 | 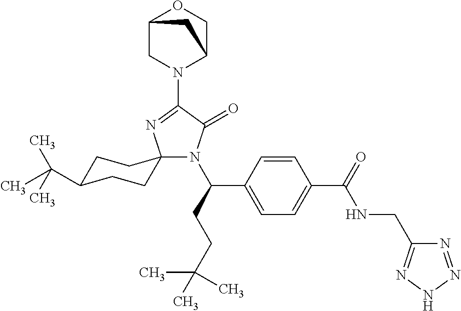 [SMILES: CC(C)(C)CC[C@H](c1ccc(C(=O)NCc2nn[nH]n2)cc1)N1C(=O)C(N2C[C@H]3C[C@@H]2CO3)=NC12CCC(C(C)(C)C)CC2] |
| 9.92 | 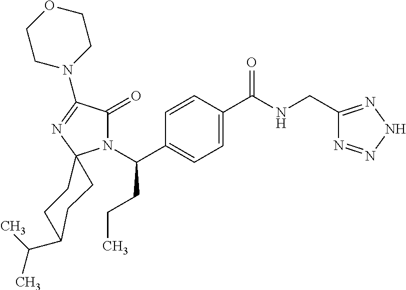 [SMILES: CCC[C@H](c1ccc(C(=O)NCc2nn[nH]n2)cc1)N1C(=O)C(N2CCOCC2)=NC12CCC(C(C)C)CC2] |
| 9.93 | 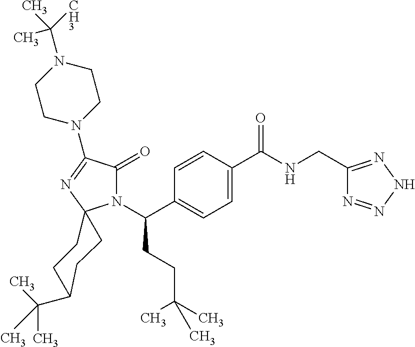 [SMILES: CC(C)(C)CC[C@H](c1ccc(C(=O)NCc2nn[nH]n2)cc1)N1C(=O)C(N2CCN(C(C)(C)C)CC2)=NC12CCC(C(C)(C)C)CC2] |
| 9.94 | 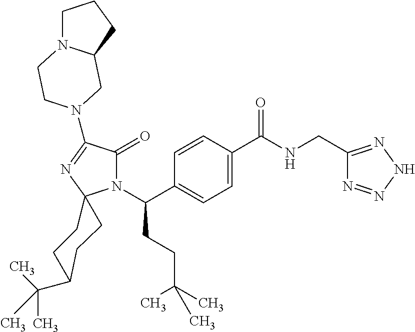 [SMILES: CC(C)(C)CC[C@H](c1ccc(C(=O)NCc2nn[nH]n2)cc1)N1C(=O)C(N2CCN3CCC[C@H]3C2)=NC12CCC(C(C)(C)C)CC2] |
| 9.95 | 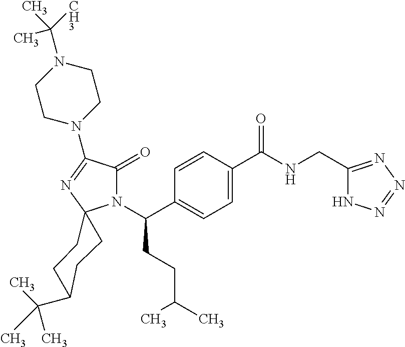 [SMILES: CC(C)CC[C@H](c1ccc(C(=O)NCc2nnn[nH]2)cc1)N1C(=O)C(N2CCN(C(C)(C)C)CC2)=NC12CCC(C(C)(C)C)CC2] |
| 9.96 | 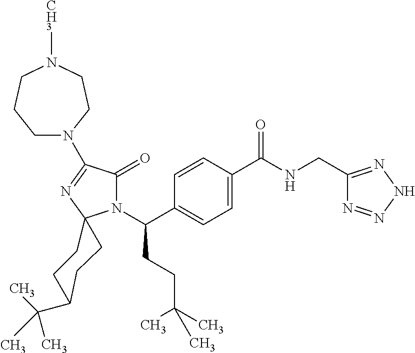 [SMILES: CN1CCCN(C2=NC3(CCC(C(C)(C)C)CC3)N([C@H](CCC(C)(C)C)c3ccc(C(=O)NCc4nn[nH]n4)cc3)C2=O)CC1] |
| 9.97 | 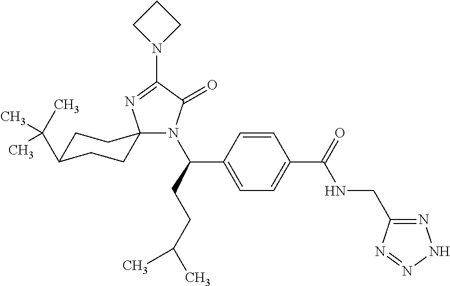 [SMILES: CC(C)CC[C@H](c1ccc(C(=O)NCc2nn[nH]n2)cc1)N1C(=O)C(N2CCC2)=NC12CCC(C(C)(C)C)CC2] |
| 9.98 | 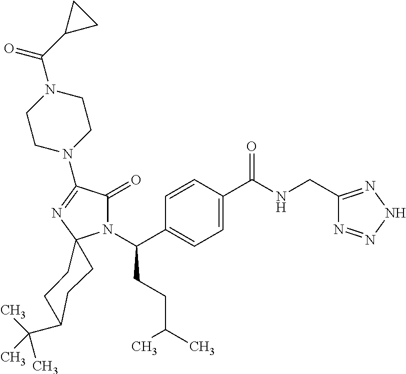 [SMILES: CC(C)CC[C@H](c1ccc(C(=O)NCc2nn[nH]n2)cc1)N1C(=O)C(N2CCN(C(=O)C3CC3)CC2)=NC12CCC(C(C)(C)C)CC2] |
| 9.99 | 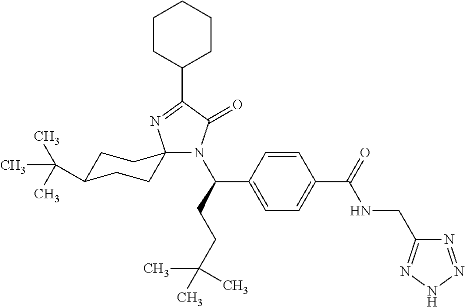 [SMILES: CC(C)(C)CC[C@H](c1ccc(C(=O)NCc2nn[nH]n2)cc1)N1C(=O)C(C2CCCCC2)=NC12CCC(C(C)(C)C)CC2] |
| 9.100 | 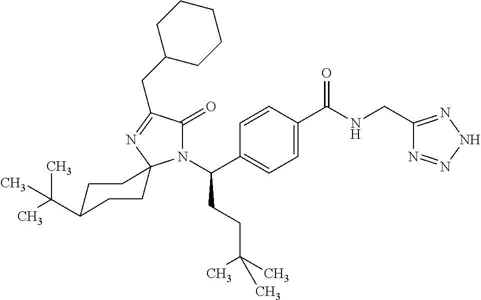 [SMILES: CC(C)(C)CC[C@H](c1ccc(C(=O)NCc2nn[nH]n2)cc1)N1C(=O)C(CC2CCCCC2)=NC12CCC(C(C)(C)C)CC2] |
| 9.101 | 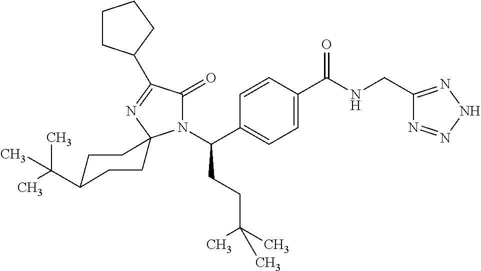 [SMILES: CC(C)(C)CC[C@H](c1ccc(C(=O)NCc2nn[nH]n2)cc1)N1C(=O)C(C2CCCC2)=NC12CCC(C(C)(C)C)CC2] |
| 9.102 | 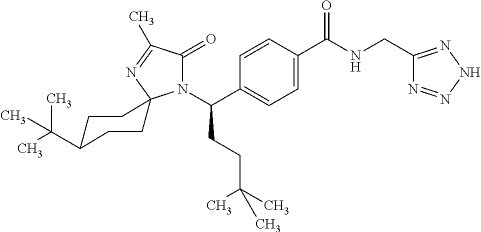 [SMILES: CC1=NC2(CCC(C(C)(C)C)CC2)N([C@H](CCC(C)(C)C)c2ccc(C(=O)NCc3nn[nH]n3)cc2)C1=O] |
| 9.103 | 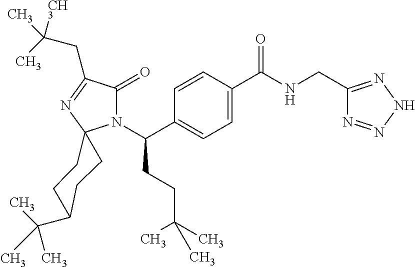 [SMILES: CC(C)(C)CC[C@H](c1ccc(C(=O)NCc2nn[nH]n2)cc1)N1C(=O)C(CC(C)(C)C)=NC12CCC(C(C)(C)C)CC2] |
| 9.104 | 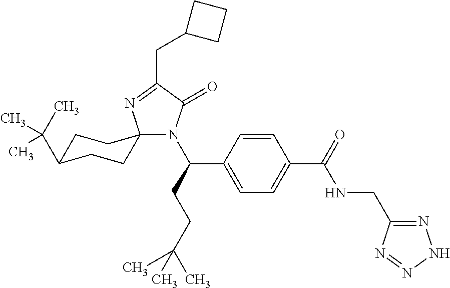 [SMILES: CC(C)(C)CC[C@H](c1ccc(C(=O)NCc2nn[nH]n2)cc1)N1C(=O)C(CC2CCC2)=NC12CCC(C(C)(C)C)CC2] |
| 9.105 | 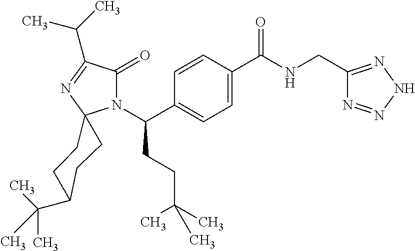 [SMILES: CC(C)C1=NC2(CCC(C(C)(C)C)CC2)N([C@H](CCC(C)(C)C)c2ccc(C(=O)NCc3nn[nH]n3)cc2)C1=O] |
| 9.106 | 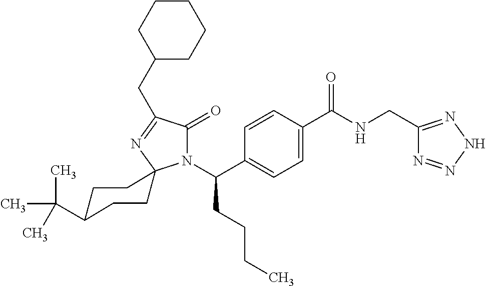 [SMILES: CCCC[C@H](c1ccc(C(=O)NCc2nn[nH]n2)cc1)N1C(=O)C(CC2CCCCC2)=NC12CCC(C(C)(C)C)CC2] |
| 9.107 | 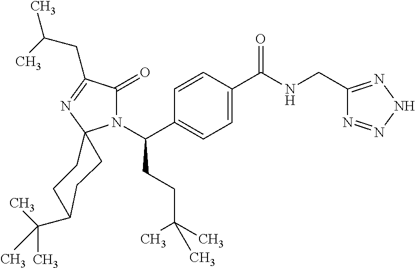 [SMILES: CC(C)CC1=NC2(CCC(C(C)(C)C)CC2)N([C@H](CCC(C)(C)C)c2ccc(C(=O)NCc3nn[nH]n3)cc2)C1=O] |
| 9.108 | 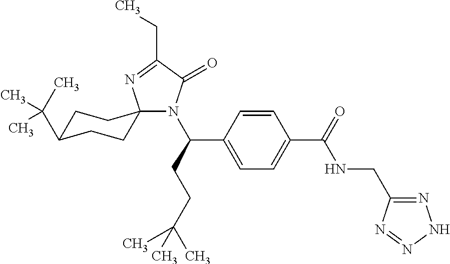 [SMILES: CCC1=NC2(CCC(C(C)(C)C)CC2)N([C@H](CCC(C)(C)C)c2ccc(C(=O)NCc3nn[nH]n3)cc2)C1=O] |
| 9.109 | 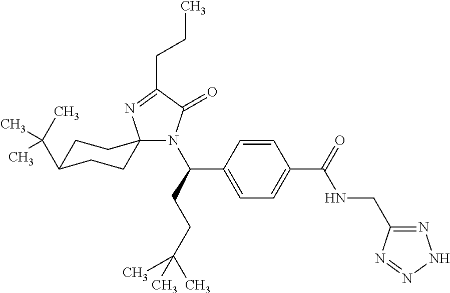 [SMILES: CCCC1=NC2(CCC(C(C)(C)C)CC2)N([C@H](CCC(C)(C)C)c2ccc(C(=O)NCc3nn[nH]n3)cc2)C1=O] |
| 9.110 | 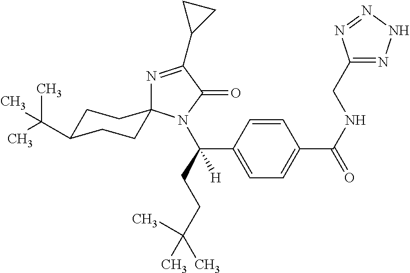 [SMILES: CC(C)(C)CC[C@H](c1ccc(C(=O)NCc2nn[nH]n2)cc1)N1C(=O)C(C2CC2)=NC12CCC(C(C)(C)C)CC2] |
| 9.111 | 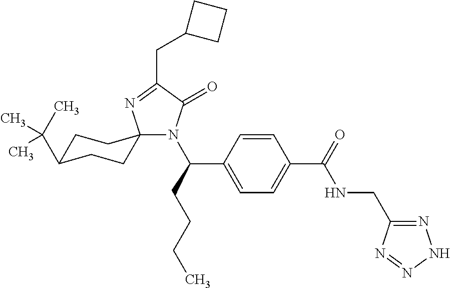 [SMILES: CCCC[C@H](c1ccc(C(=O)NCc2nn[nH]n2)cc1)N1C(=O)C(CC2CCC2)=NC12CCC(C(C)(C)C)CC2] |
| 9.112 | 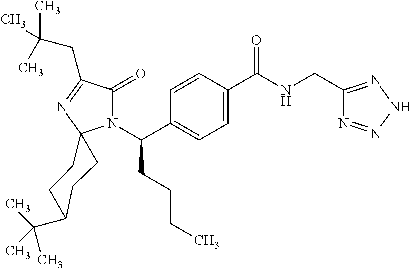 [SMILES: CCCC[C@H](c1ccc(C(=O)NCc2nn[nH]n2)cc1)N1C(=O)C(CC(C)(C)C)=NC12CCC(C(C)(C)C)CC2] |
| 9.113 | 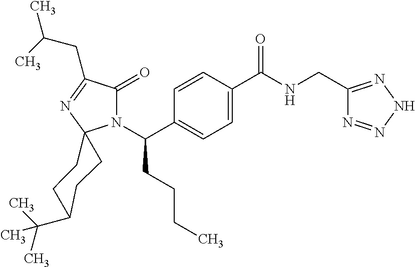 [SMILES: CCCC[C@H](c1ccc(C(=O)NCc2nn[nH]n2)cc1)N1C(=O)C(CC(C)C)=NC12CCC(C(C)(C)C)CC2] |
| 9.114 | 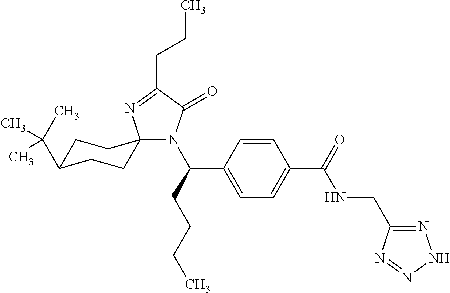 [SMILES: CCCC[C@H](c1ccc(C(=O)NCc2nn[nH]n2)cc1)N1C(=O)C(CCC)=NC12CCC(C(C)(C)C)CC2] |
| 9.115 | 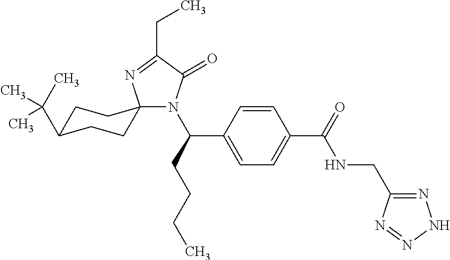 [SMILES: CCCC[C@H](c1ccc(C(=O)NCc2nn[nH]n2)cc1)N1C(=O)C(CC)=NC12CCC(C(C)(C)C)CC2] |
| 9.116 | 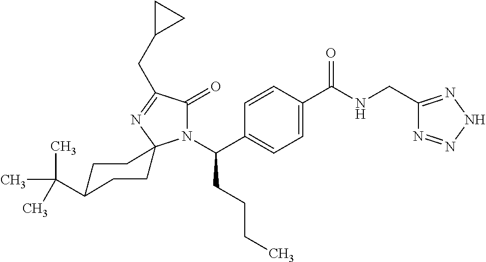 [SMILES: CCCC[C@H](c1ccc(C(=O)NCc2nn[nH]n2)cc1)N1C(=O)C(CC2CC2)=NC12CCC(C(C)(C)C)CC2] |
| 9.117 | 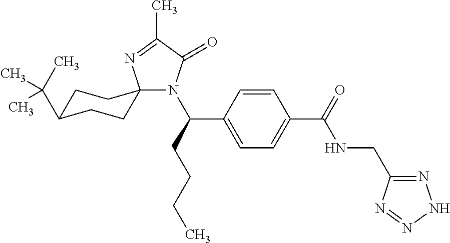 [SMILES: CCCC[C@H](c1ccc(C(=O)NCc2nn[nH]n2)cc1)N1C(=O)C(C)=NC12CCC(C(C)(C)C)CC2] |
| 9.118 | 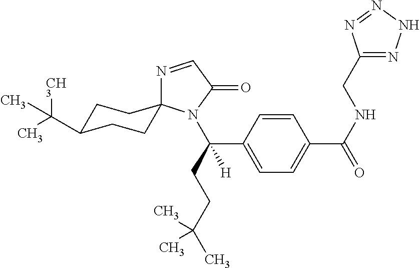 [SMILES: CC(C)(C)CC[C@H](c1ccc(C(=O)NCc2nn[nH]n2)cc1)N1C(=O)C=NC12CCC(C(C)(C)C)CC2] |
| 9.119 | 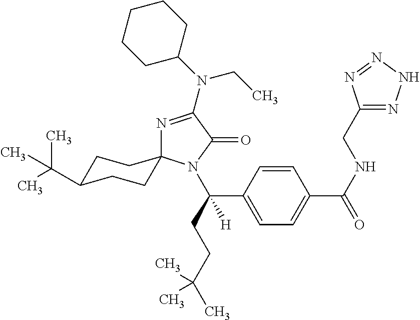 [SMILES: CCN(C1=NC2(CCC(C(C)(C)C)CC2)N([C@H](CCC(C)(C)C)c2ccc(C(=O)NCc3nn[nH]n3)cc2)C1=O)C1CCCCC1] |
| 9.120 | 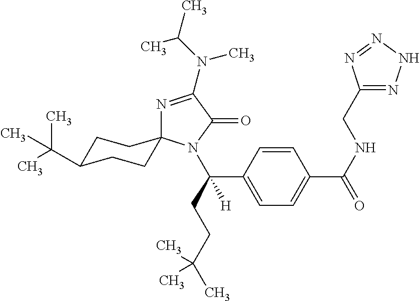 [SMILES: CC(C)N(C)C1=NC2(CCC(C(C)(C)C)CC2)N([C@H](CCC(C)(C)C)c2ccc(C(=O)NCc3nn[nH]n3)cc2)C1=O] |
| 9.121 | 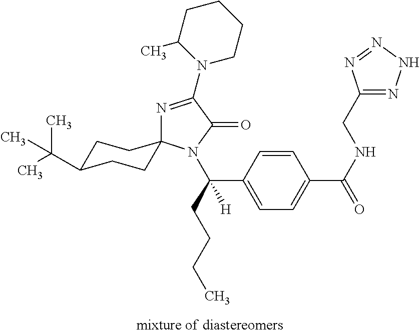 [SMILES: CCCC[C@H](c1ccc(C(=O)NCc2nn[nH]n2)cc1)N1C(=O)C(N2CCCCC2C)=NC12CCC(C(C)(C)C)CC2] |
| 9.122 | 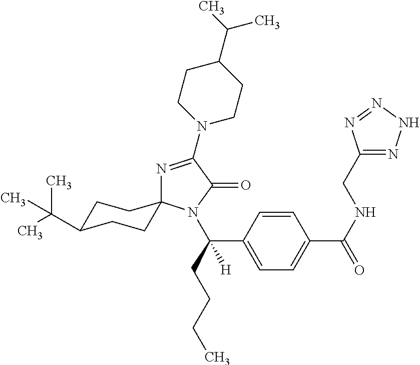 [SMILES: CCCC[C@H](c1ccc(C(=O)NCc2nn[nH]n2)cc1)N1C(=O)C(N2CCC(C(C)C)CC2)=NC12CCC(C(C)(C)C)CC2] |
| 9.123 | 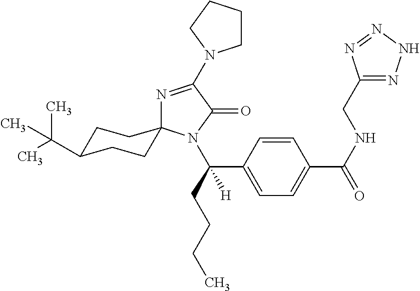 [SMILES: CCCC[C@H](c1ccc(C(=O)NCc2nn[nH]n2)cc1)N1C(=O)C(N2CCCC2)=NC12CCC(C(C)(C)C)CC2] |
| 9.124 | 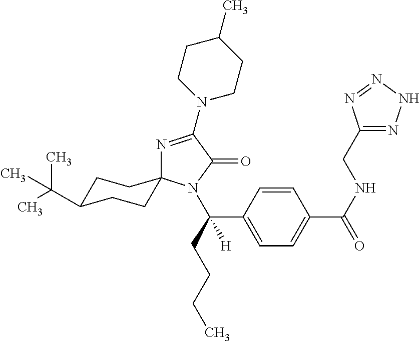 [SMILES: CCCC[C@H](c1ccc(C(=O)NCc2nn[nH]n2)cc1)N1C(=O)C(N2CCC(C)CC2)=NC12CCC(C(C)(C)C)CC2] |
| 9.125 | 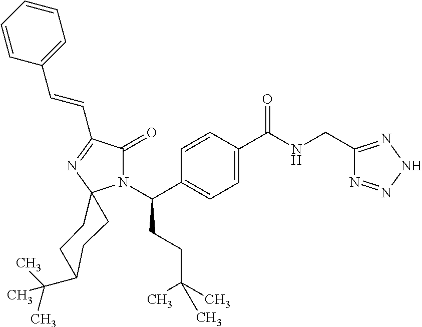 [SMILES: CC(C)(C)CC[C@H](c1ccc(C(=O)NCc2nn[nH]n2)cc1)N1C(=O)C(/C=C/c2ccccc2)=NC12CCC(C(C)(C)C)CC2] |
| 9.126 | 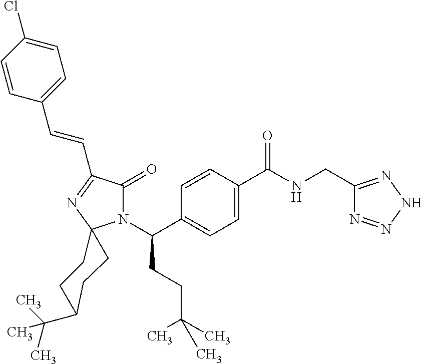 [SMILES: CC(C)(C)CC[C@H](c1ccc(C(=O)NCc2nn[nH]n2)cc1)N1C(=O)C(/C=C/c2ccc(Cl)cc2)=NC12CCC(C(C)(C)C)CC2] |
| 9.127 | 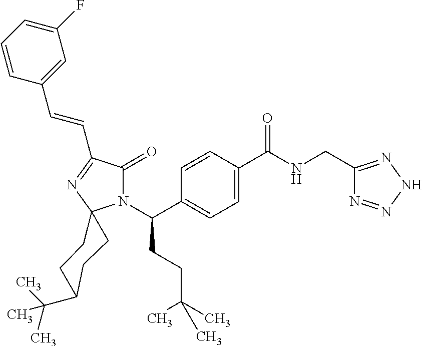 [SMILES: CC(C)(C)CC[C@H](c1ccc(C(=O)NCc2nn[nH]n2)cc1)N1C(=O)C(/C=C/c2cccc(F)c2)=NC12CCC(C(C)(C)C)CC2] |
| 9.128 | 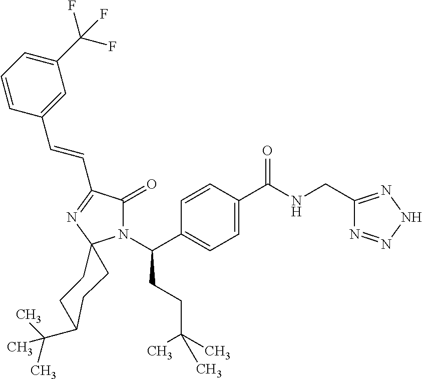 [SMILES: CC(C)(C)CC[C@H](c1ccc(C(=O)NCc2nn[nH]n2)cc1)N1C(=O)C(/C=C/c2cccc(C(F)(F)F)c2)=NC12CCC(C(C)(C)C)CC2] |
| 9.129 | 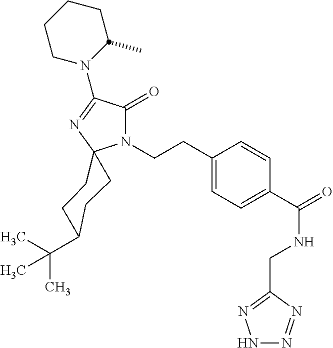 |
| 9.130 | 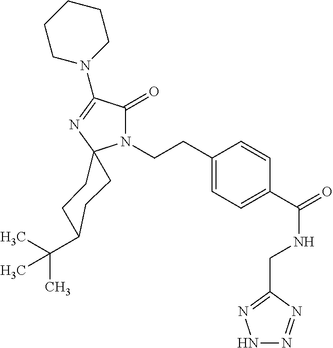 [SMILES: CC(C)(C)C1CCC2(CC1)N=C(N1CCCCC1)C(=O)N2CCc1ccc(C(=O)NCc2nn[nH]n2)cc1] |
| 10.1 | 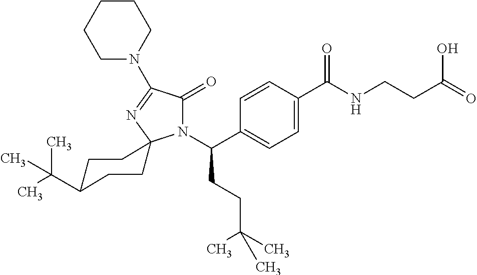 [SMILES: CC(C)(C)CC[C@H](c1ccc(C(=O)NCCC(=O)O)cc1)N1C(=O)C(N2CCCCC2)=NC12CCC(C(C)(C)C)CC2] |
| 10.2 | 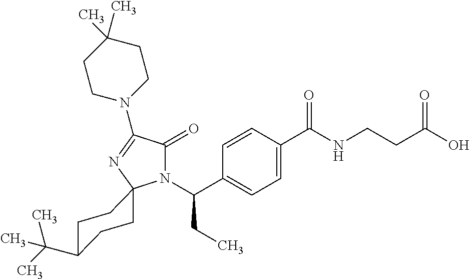 [SMILES: CC[C@H](c1ccc(C(=O)NCCC(=O)O)cc1)N1C(=O)C(N2CCC(C)(C)CC2)=NC12CCC(C(C)(C)C)CC2] |
| 10.3 | 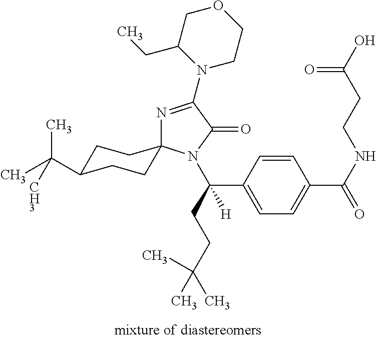 [SMILES: CCC1COCCN1C1=NC2(CCC(C(C)(C)C)CC2)N([C@H](CCC(C)(C)C)c2ccc(C(=O)NCCC(=O)O)cc2)C1=O] |
| 10.4 | 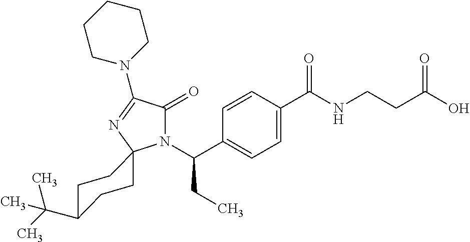 [SMILES: CC[C@H](c1ccc(C(=O)NCCC(=O)O)cc1)N1C(=O)C(N2CCCCC2)=NC12CCC(C(C)(C)C)CC2] |
| 10.5 | 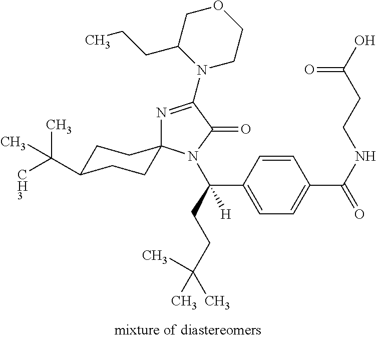 [SMILES: CCCC1COCCN1C1=NC2(CCC(C(C)(C)C)CC2)N([C@H](CCC(C)(C)C)c2ccc(C(=O)NCCC(=O)O)cc2)C1=O] |
| 10.6 | 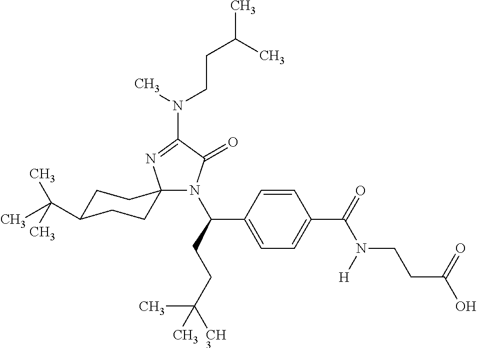 [SMILES: CC(C)CCN(C)C1=NC2(CCC(C(C)(C)C)CC2)N([C@H](CCC(C)(C)C)c2ccc(C(=O)NCCC(=O)O)cc2)C1=O] |
| 10.7 | 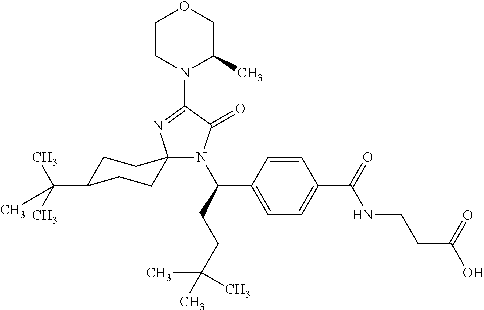 [SMILES: C[C@@H]1COCCN1C1=NC2(CCC(C(C)(C)C)CC2)N([C@H](CCC(C)(C)C)c2ccc(C(=O)NCCC(=O)O)cc2)C1=O] |
| 10.8 | 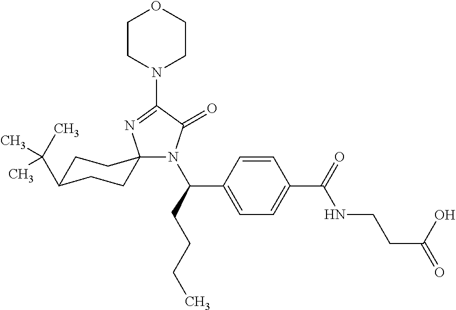 [SMILES: CCCC[C@H](c1ccc(C(=O)NCCC(=O)O)cc1)N1C(=O)C(N2CCOCC2)=NC12CCC(C(C)(C)C)CC2] |
| 10.9 | 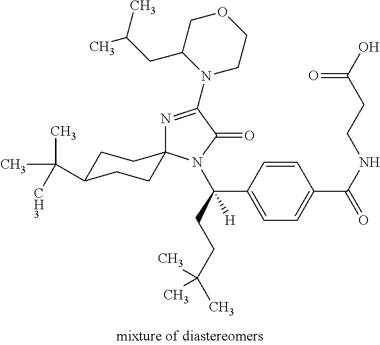 [SMILES: CC(C)CC1COCCN1C1=NC2(CCC(C(C)(C)C)CC2)N([C@H](CCC(C)(C)C)c2ccc(C(=O)NCCC(=O)O)cc2)C1=O] |
| 10.10 | 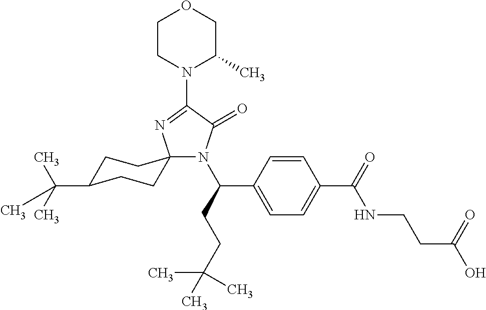 [SMILES: C[C@H]1COCCN1C1=NC2(CCC(C(C)(C)C)CC2)N([C@H](CCC(C)(C)C)c2ccc(C(=O)NCCC(=O)O)cc2)C1=O] |
| 10.11 | 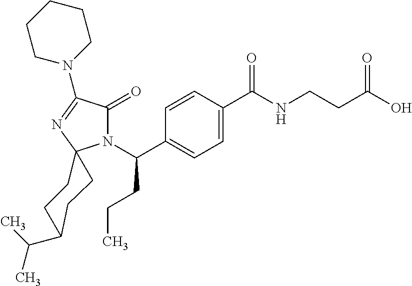 [SMILES: CCC[C@H](c1ccc(C(=O)NCCC(=O)O)cc1)N1C(=O)C(N2CCCCC2)=NC12CCC(C(C)C)CC2] |
| 10.12 | 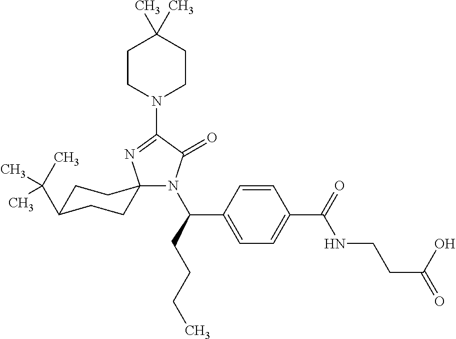 [SMILES: CCCC[C@H](c1ccc(C(=O)NCCC(=O)O)cc1)N1C(=O)C(N2CCC(C)(C)CC2)=NC12CCC(C(C)(C)C)CC2] |
| 10.13 | 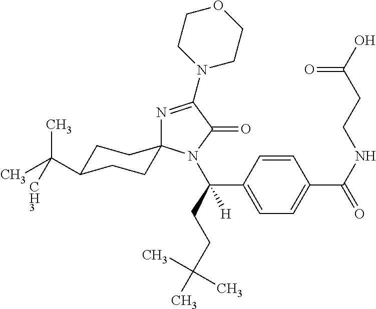 [SMILES: CC(C)(C)CC[C@H](c1ccc(C(=O)NCCC(=O)O)cc1)N1C(=O)C(N2CCOCC2)=NC12CCC(C(C)(C)C)CC2] |
| 10.14 | 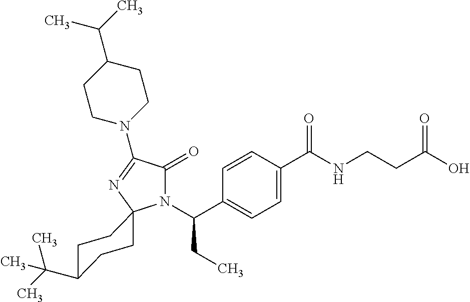 [SMILES: CC[C@H](c1ccc(C(=O)NCCC(=O)O)cc1)N1C(=O)C(N2CCC(C(C)C)CC2)=NC12CCC(C(C)(C)C)CC2] |
| 10.15 | 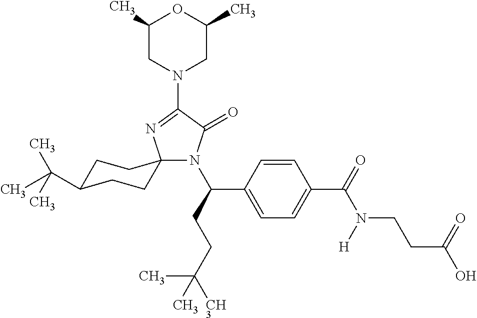 [SMILES: C[C@@H]1CN(C2=NC3(CCC(C(C)(C)C)CC3)N([C@H](CCC(C)(C)C)c3ccc(C(=O)NCCC(=O)O)cc3)C2=O)C[C@H](C)O1] |
| 10.16 | 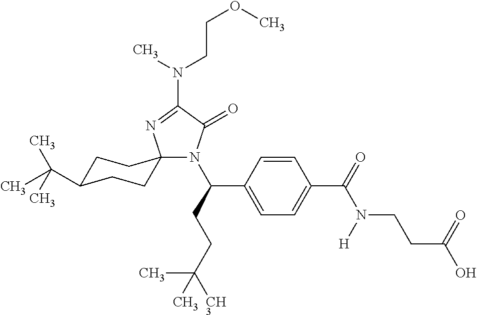 [SMILES: COCCN(C)C1=NC2(CCC(C(C)(C)C)CC2)N([C@H](CCC(C)(C)C)c2ccc(C(=O)NCCC(=O)O)cc2)C1=O] |
| 10.17 | 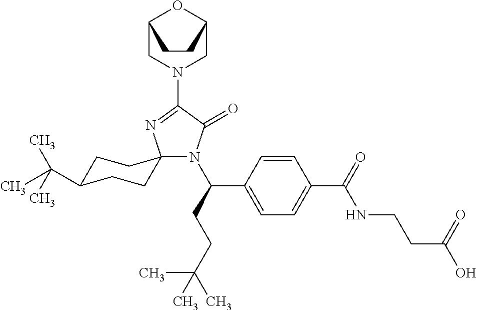 [SMILES: CC(C)(C)CC[C@H](c1ccc(C(=O)NCCC(=O)O)cc1)N1C(=O)C(N2C[C@H]3CC[C@@H](C2)O3)=NC12CCC(C(C)(C)C)CC2] |
| 10.18 | 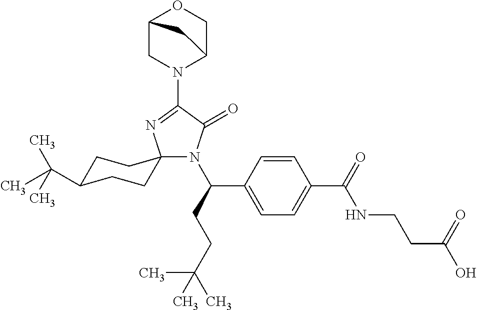 [SMILES: CC(C)(C)CC[C@H](c1ccc(C(=O)NCCC(=O)O)cc1)N1C(=O)C(N2C[C@H]3CC2CO3)=NC12CCC(C(C)(C)C)CC2] |
| 10.19 | 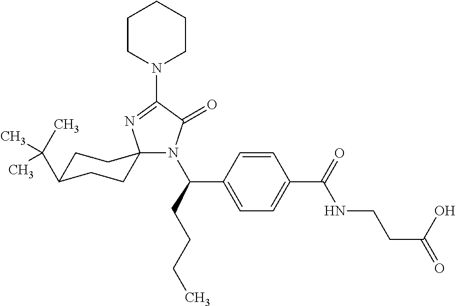 [SMILES: CCCC[C@H](c1ccc(C(=O)NCCC(=O)O)cc1)N1C(=O)C(N2CCCCC2)=NC12CCC(C(C)(C)C)CC2] |
| 10.20 | 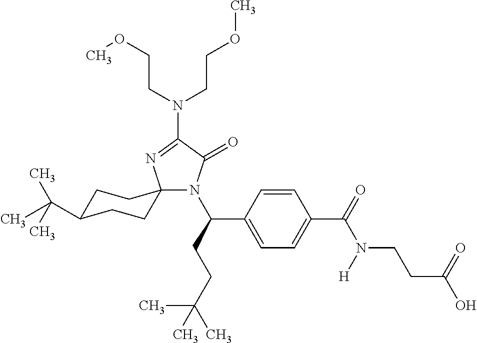 [SMILES: COCCN(CCOC)C1=NC2(CCC(C(C)(C)C)CC2)N([C@H](CCC(C)(C)C)c2ccc(C(=O)NCCC(=O)O)cc2)C1=O] |
| 10.21 | 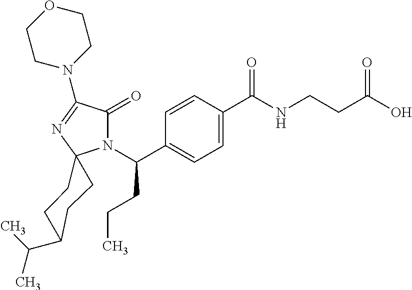 [SMILES: CCC[C@H](c1ccc(C(=O)NCCC(=O)O)cc1)N1C(=O)C(N2CCOCC2)=NC12CCC(C(C)C)CC2] |
| 10.22 | 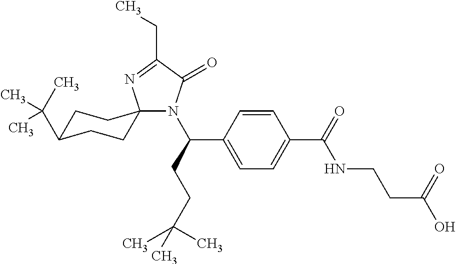 [SMILES: CCC1=NC2(CCC(C(C)(C)C)CC2)N([C@H](CCC(C)(C)C)c2ccc(C(=O)NCCC(=O)O)cc2)C1=O] |
| 10.23 | 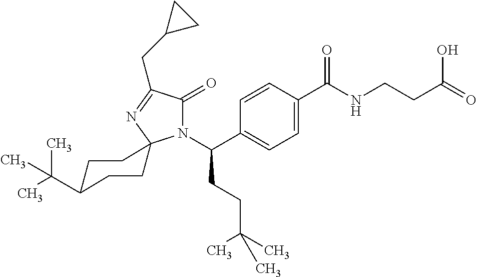 [SMILES: CC(C)(C)CC[C@H](c1ccc(C(=O)NCCC(=O)O)cc1)N1C(=O)C(CC2CC2)=NC12CCC(C(C)(C)C)CC2] |
| 10.24 | 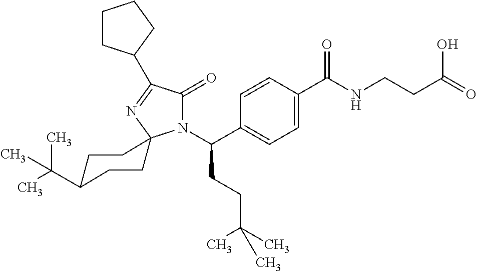 [SMILES: CC(C)(C)CC[C@H](c1ccc(C(=O)NCCC(=O)O)cc1)N1C(=O)C(C2CCCC2)=NC12CCC(C(C)(C)C)CC2] |
| 10.25 | 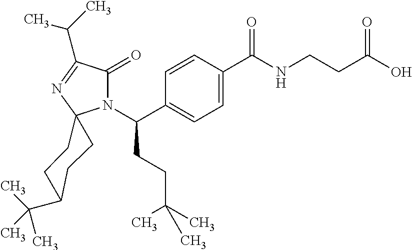 [SMILES: CC(C)C1=NC2(CCC(C(C)(C)C)CC2)N([C@H](CCC(C)(C)C)c2ccc(C(=O)NCCC(=O)O)cc2)C1=O] |
| 10.26 | 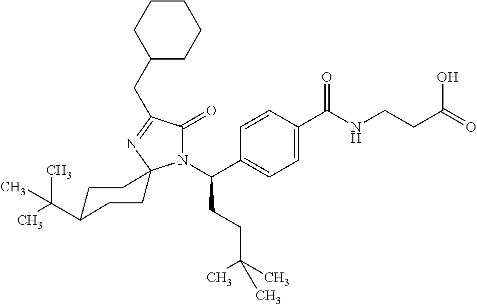 [SMILES: CC(C)(C)CC[C@H](c1ccc(C(=O)NCCC(=O)O)cc1)N1C(=O)C(CC2CCCCC2)=NC12CCC(C(C)(C)C)CC2] |
| 10.27 | 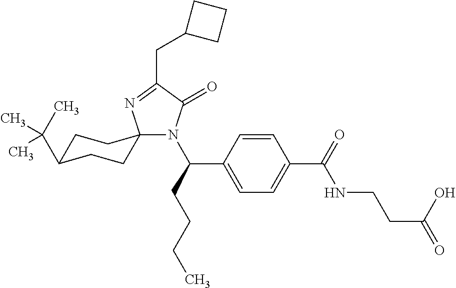 [SMILES: CCCC[C@H](c1ccc(C(=O)NCCC(=O)O)cc1)N1C(=O)C(CC2CCC2)=NC12CCC(C(C)(C)C)CC2] |
| 10.28 | 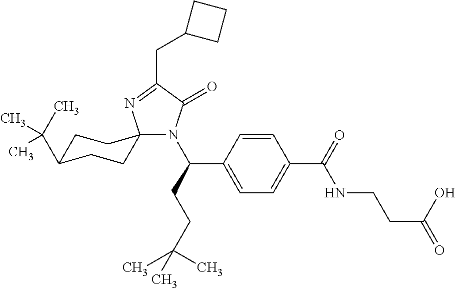 [SMILES: CC(C)(C)CC[C@H](c1ccc(C(=O)NCCC(=O)O)cc1)N1C(=O)C(CC2CCC2)=NC12CCC(C(C)(C)C)CC2] |
| 10.29 | 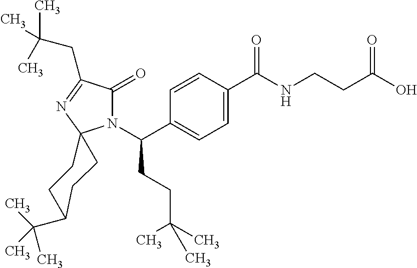 [SMILES: CC(C)(C)CC[C@H](c1ccc(C(=O)NCCC(=O)O)cc1)N1C(=O)C(CC(C)(C)C)=NC12CCC(C(C)(C)C)CC2] |
| 10.30 | 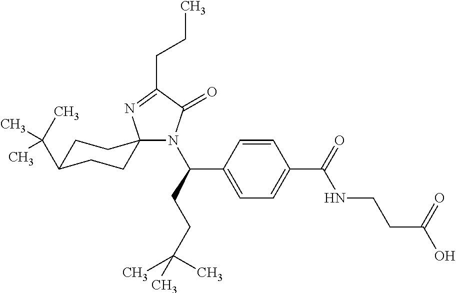 [SMILES: CCCC1=NC2(CCC(C(C)(C)C)CC2)N([C@H](CCC(C)(C)C)c2ccc(C(=O)NCCC(=O)O)cc2)C1=O] |
| 10.31 | 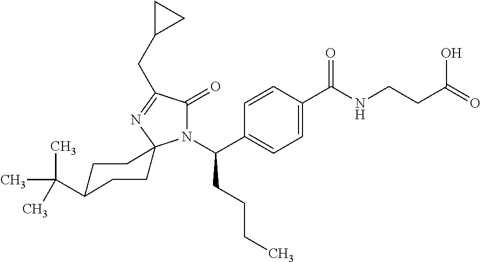 [SMILES: CCCC[C@H](c1ccc(C(=O)NCCC(=O)O)cc1)N1C(=O)C(CC2CC2)=NC12CCC(C(C)(C)C)CC2] |
| 10.32 | 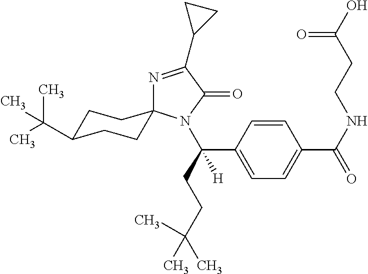 [SMILES: CC(C)(C)CC[C@H](c1ccc(C(=O)NCCC(=O)O)cc1)N1C(=O)C(C2CC2)=NC12CCC(C(C)(C)C)CC2] |
| 10.33 | 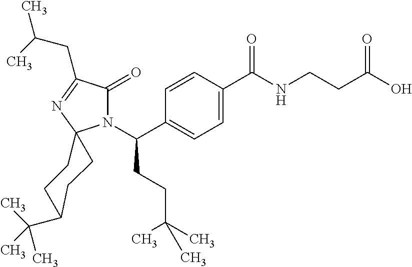 [SMILES: CC(C)CC1=NC2(CCC(C(C)(C)C)CC2)N([C@H](CCC(C)(C)C)c2ccc(C(=O)NCCC(=O)O)cc2)C1=O] |
| 10.34 | 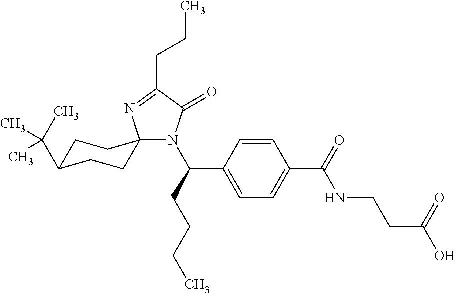 [SMILES: CCCC[C@H](c1ccc(C(=O)NCCC(=O)O)cc1)N1C(=O)C(CCC)=NC12CCC(C(C)(C)C)CC2] |
| 10.35 | 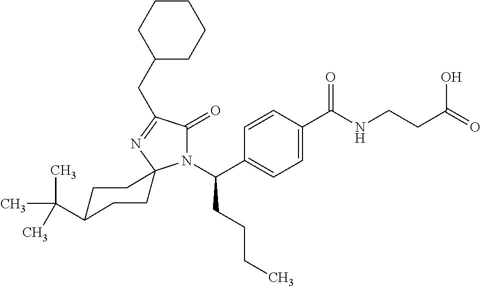 [SMILES: CCCC[C@H](c1ccc(C(=O)NCCC(=O)O)cc1)N1C(=O)C(CC2CCCCC2)=NC12CCC(C(C)(C)C)CC2] |
| 10.36 | 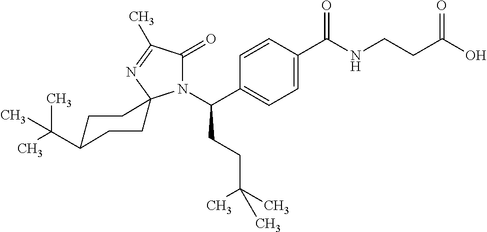 [SMILES: CC1=NC2(CCC(C(C)(C)C)CC2)N([C@H](CCC(C)(C)C)c2ccc(C(=O)NCCC(=O)O)cc2)C1=O] |
| 10.37 | 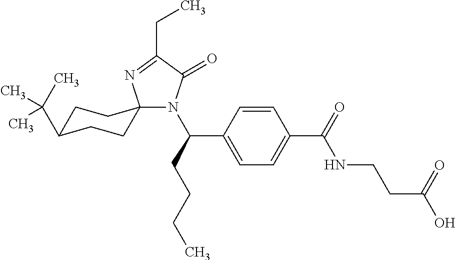 [SMILES: CCCC[C@H](c1ccc(C(=O)NCCC(=O)O)cc1)N1C(=O)C(CC)=NC12CCC(C(C)(C)C)CC2] |
| 10.38 | 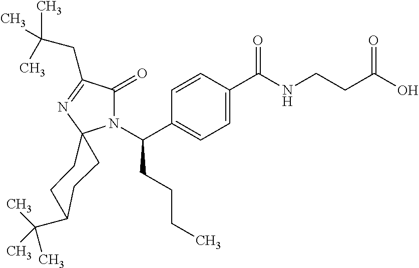 [SMILES: CCCC[C@H](c1ccc(C(=O)NCCC(=O)O)cc1)N1C(=O)C(CC(C)(C)C)=NC12CCC(C(C)(C)C)CC2] |
| 10.39 | 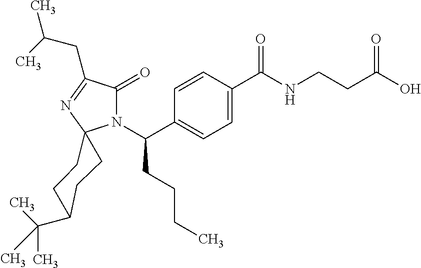 [SMILES: CCCC[C@H](c1ccc(C(=O)NCCC(=O)O)cc1)N1C(=O)C(CC(C)C)=NC12CCC(C(C)(C)C)CC2] |
| 10.40 | 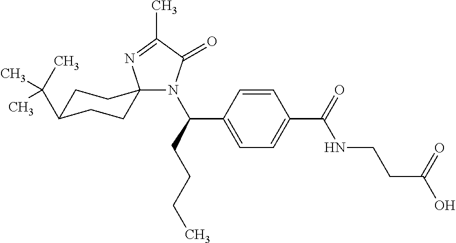 [SMILES: CCCC[C@H](c1ccc(C(=O)NCCC(=O)O)cc1)N1C(=O)C(C)=NC12CCC(C(C)(C)C)CC2] |
| 10.41 | 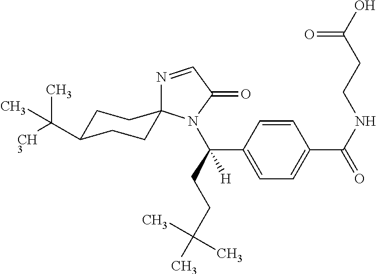 [SMILES: CC(C)(C)CC[C@H](c1ccc(C(=O)NCCC(=O)O)cc1)N1C(=O)C=NC12CCC(C(C)(C)C)CC2] |
| 10.42 | 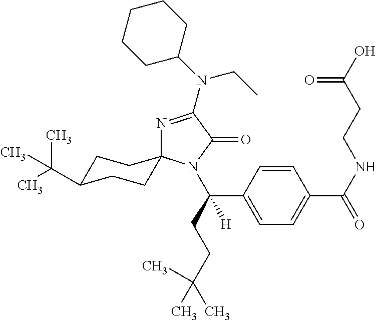 [SMILES: CCN(C1=NC2(CCC(C(C)(C)C)CC2)N([C@H](CCC(C)(C)C)c2ccc(C(=O)NCCC(=O)O)cc2)C1=O)C1CCCCC1] |
| 10.43 | 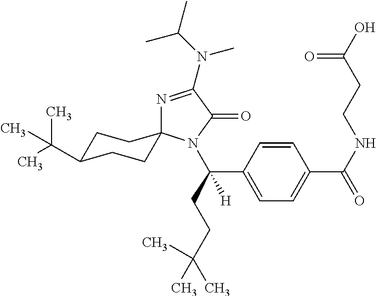 [SMILES: CC(C)N(C)C1=NC2(CCC(C(C)(C)C)CC2)N([C@H](CCC(C)(C)C)c2ccc(C(=O)NCCC(=O)O)cc2)C1=O] |
| 10.44 | 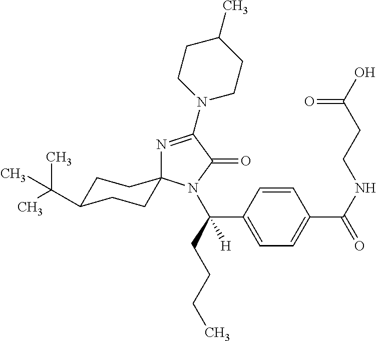 [SMILES: CCCC[C@H](c1ccc(C(=O)NCCC(=O)O)cc1)N1C(=O)C(N2CCC(C)CC2)=NC12CCC(C(C)(C)C)CC2] |
| 10.45 | 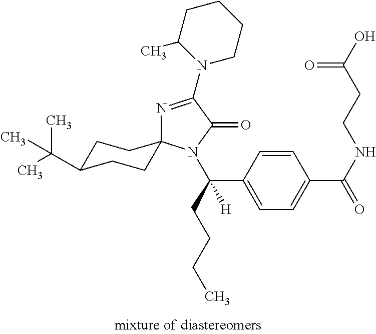 [SMILES: CCCC[C@H](c1ccc(C(=O)NCCC(=O)O)cc1)N1C(=O)C(N2CCCCC2C)=NC12CCC(C(C)(C)C)CC2] |
| 10.46 | 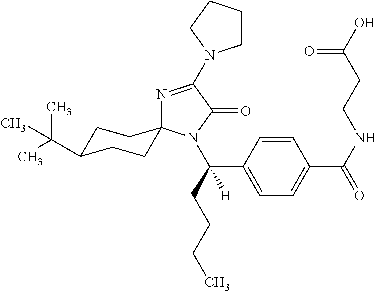 [SMILES: CCCC[C@H](c1ccc(C(=O)NCCC(=O)O)cc1)N1C(=O)C(N2CCCC2)=NC12CCC(C(C)(C)C)CC2] |
| 10.47 | 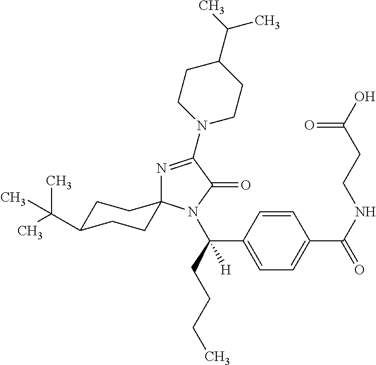 [SMILES: CCCC[C@H](c1ccc(C(=O)NCCC(=O)O)cc1)N1C(=O)C(N2CCC(C(C)C)CC2)=NC12CCC(C(C)(C)C)CC2] |
| 10.48 | 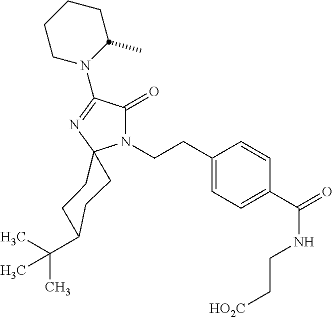 |
| 10.49 | 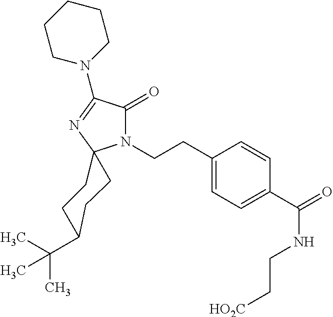 [SMILES: CC(C)(C)C1CCC2(CC1)N=C(N1CCCCC1)C(=O)N2CCc1ccc(C(=O)NCCC(=O)O)cc1] |
| 11.1 | 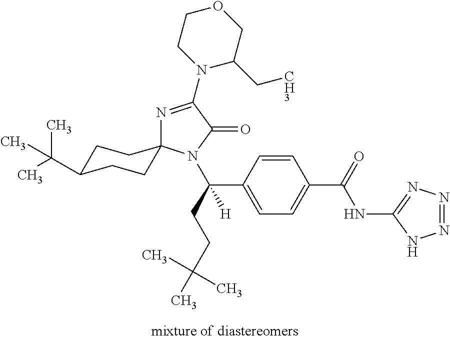 [SMILES: CCC1COCCN1C1=NC2(CCC(C(C)(C)C)CC2)N([C@H](CCC(C)(C)C)c2ccc(C(=O)Nc3nnn[nH]3)cc2)C1=O] |
| 11.2 | 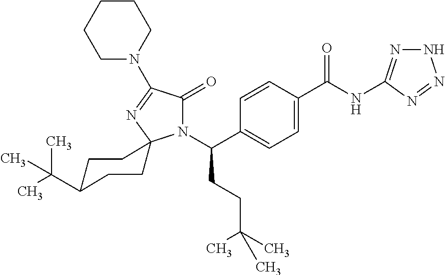 [SMILES: CC(C)(C)CC[C@H](c1ccc(C(=O)Nc2nn[nH]n2)cc1)N1C(=O)C(N2CCCCC2)=NC12CCC(C(C)(C)C)CC2] |

14. A compound of claim 1, or a pharmaceutically acceptable salt or tautomer of said compound, said compound selected from the group consisting of:

| Ex. | Structure |
|---|---|
| 9.1 | 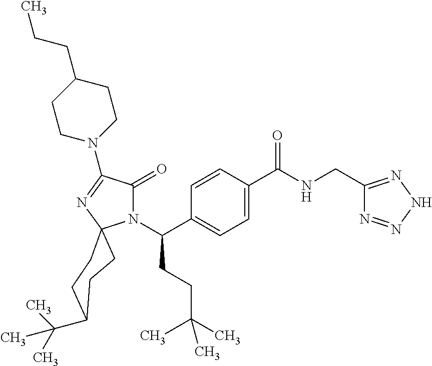 [SMILES: CCCC1CCN(C2=NC3(CCC(C(C)(C)C)CC3)N([C@H](CCC(C)(C)C)c3ccc(C(=O)NCc4nn[nH]n4)cc3)C2=O)CC1] |
| 9.2 | 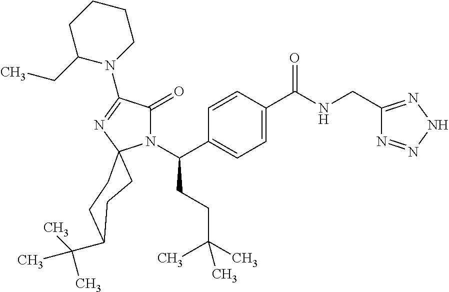 [SMILES: CCC1CCCCN1C1=NC2(CCC(C(C)(C)C)CC2)N([C@H](CCC(C)(C)C)c2ccc(C(=O)NCc3nn[nH]n3)cc2)C1=O] mixture of diastereomers |
| 9.3 | 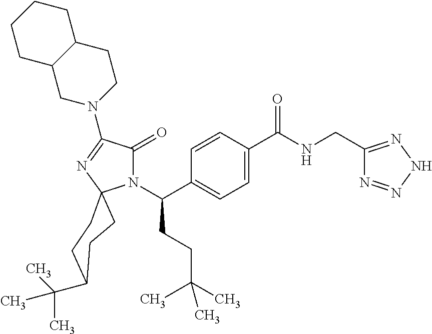 [SMILES: CC(C)(C)CC[C@H](c1ccc(C(=O)NCc2nn[nH]n2)cc1)N1C(=O)C(N2CCC3CCCCC3C2)=NC12CCC(C(C)(C)C)CC2] mixture of diastereomers |
| 9.4 | 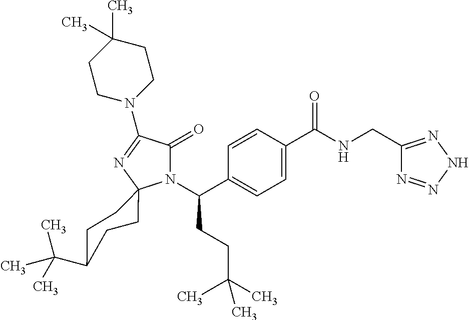 [SMILES: CC(C)(C)CC[C@H](c1ccc(C(=O)NCc2nn[nH]n2)cc1)N1C(=O)C(N2CCC(C)(C)CC2)=NC12CCC(C(C)(C)C)CC2] |
| 9.5 | 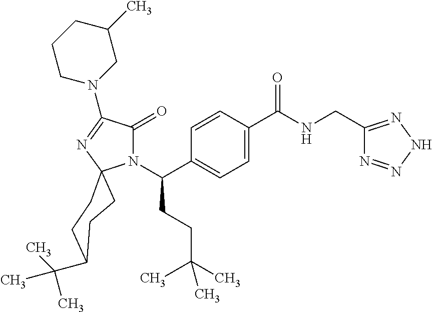 [SMILES: CC1CCCN(C2=NC3(CCC(C(C)(C)C)CC3)N([C@H](CCC(C)(C)C)c3ccc(C(=O)NCc4nn[nH]n4)cc3)C2=O)C1] mixture of diastereomers |
| 9.6 | 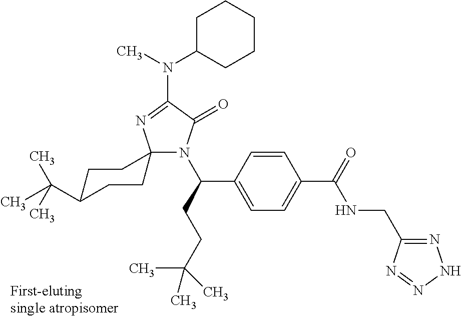 [SMILES: CN(C1=NC2(CCC(C(C)(C)C)CC2)N([C@H](CCC(C)(C)C)c2ccc(C(=O)NCc3nn[nH]n3)cc2)C1=O)C1CCCCC1] |
| 9.7 | 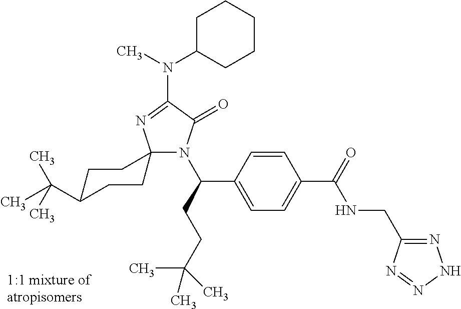 [SMILES: CN(C1=NC2(CCC(C(C)(C)C)CC2)N([C@H](CCC(C)(C)C)c2ccc(C(=O)NCc3nn[nH]n3)cc2)C1=O)C1CCCCC1] |
| 9.8 | 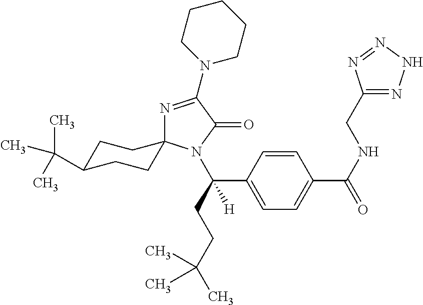 [SMILES: CC(C)(C)CC[C@H](c1ccc(C(=O)NCc2nn[nH]n2)cc1)N1C(=O)C(N2CCCCC2)=NC12CCC(C(C)(C)C)CC2] |
| 9.9 | 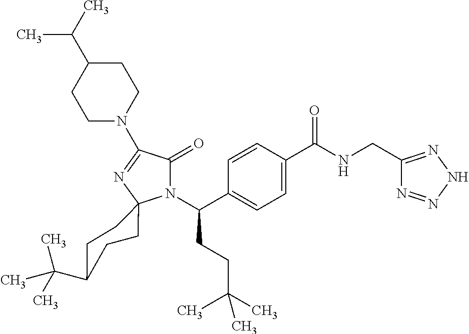 [SMILES: CC(C)C1CCN(C2=NC3(CCC(C(C)(C)C)CC3)N([C@H](CCC(C)(C)C)c3ccc(C(=O)NCc4nn[nH]n4)cc3)C2=O)CC1] |
| 9.10 | 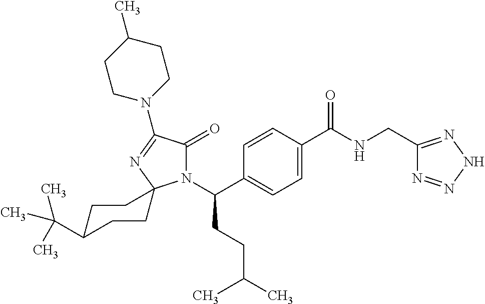 [SMILES: CC(C)CC[C@H](c1ccc(C(=O)NCc2nn[nH]n2)cc1)N1C(=O)C(N2CCC(C)CC2)=NC12CCC(C(C)(C)C)CC2] |
| 9.11 | 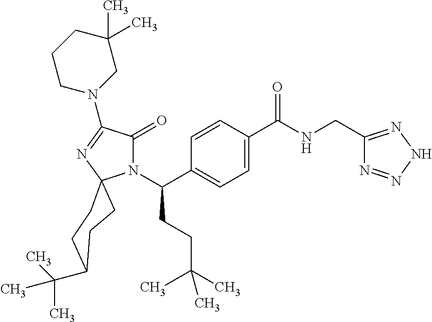 [SMILES: CC(C)(C)CC[C@H](c1ccc(C(=O)NCc2nn[nH]n2)cc1)N1C(=O)C(N2CCCC(C)(C)C2)=NC12CCC(C(C)(C)C)CC2] |
| 9.12 | 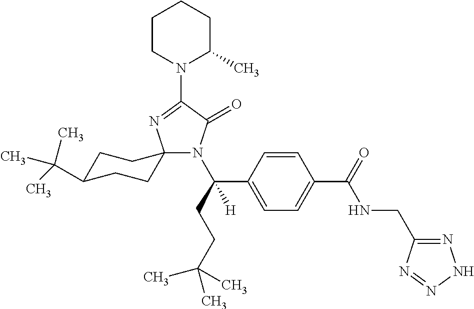 [SMILES: C[C@H]1CCCCN1C1=NC2(CCC(C(C)(C)C)CC2)N([C@H](CCC(C)(C)C)c2ccc(C(=O)NCc3nn[nH]n3)cc2)C1=O] |
| 9.13 | 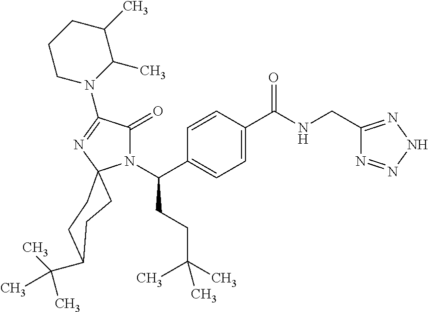 [SMILES: CC1CCCN(C2=NC3(CCC(C(C)(C)C)CC3)N([C@H](CCC(C)(C)C)c3ccc(C(=O)NCc4nn[nH]n4)cc3)C2=O)C1C] mixture of diastereomers |
| 9.14 | 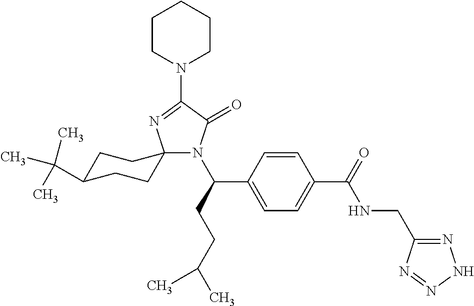 [SMILES: CC(C)CC[C@H](c1ccc(C(=O)NCc2nn[nH]n2)cc1)N1C(=O)C(N2CCCCC2)=NC12CCC(C(C)(C)C)CC2] |
| 9.15 | 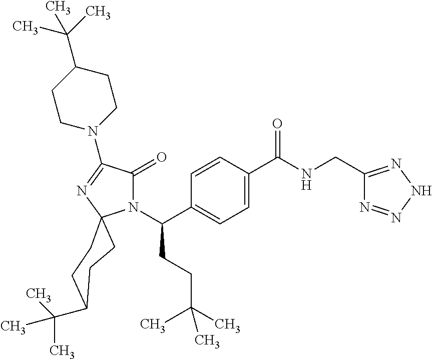 [SMILES: CC(C)(C)CC[C@H](c1ccc(C(=O)NCc2nn[nH]n2)cc1)N1C(=O)C(N2CCC(C(C)(C)C)CC2)=NC12CCC(C(C)(C)C)CC2] |
| 9.16 | 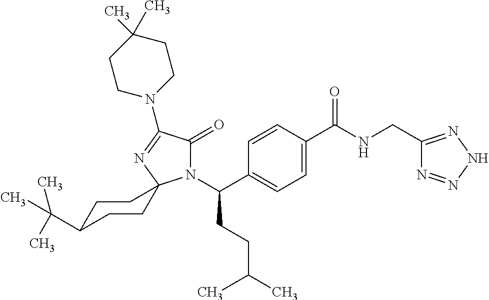 [SMILES: CC(C)CC[C@H](c1ccc(C(=O)NCc2nn[nH]n2)cc1)N1C(=O)C(N2CCC(C)(C)CC2)=NC12CCC(C(C)(C)C)CC2] |
| 9.17 | 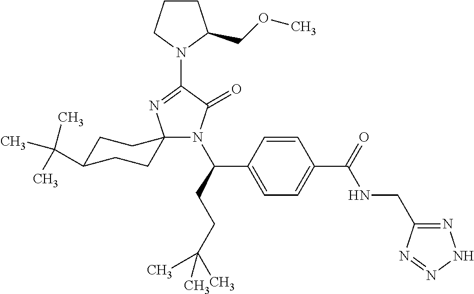 [SMILES: COC[C@@H]1CCCN1C1=NC2(CCC(C(C)(C)C)CC2)N([C@H](CCC(C)(C)C)c2ccc(C(=O)NCc3nn[nH]n3)cc2)C1=O] |
| 9.18 | 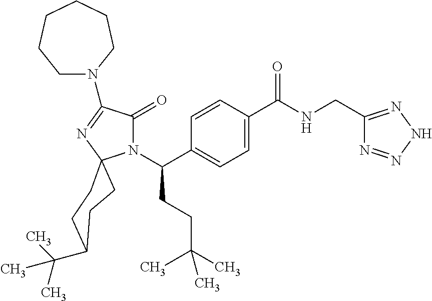 [SMILES: CC(C)(C)CC[C@H](c1ccc(C(=O)NCc2nn[nH]n2)cc1)N1C(=O)C(N2CCCCCC2)=NC12CCC(C(C)(C)C)CC2] |
| 9.19 | 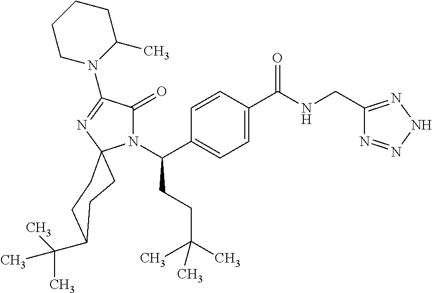 [SMILES: CC1CCCCN1C1=NC2(CCC(C(C)(C)C)CC2)N([C@H](CCC(C)(C)C)c2ccc(C(=O)NCc3nn[nH]n3)cc2)C1=O] mixture of diastereomers |
| 9.20 | 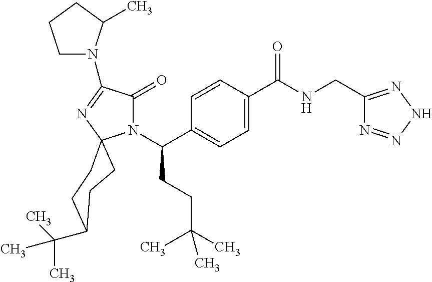 [SMILES: CC1CCCN1C1=NC2(CCC(C(C)(C)C)CC2)N([C@H](CCC(C)(C)C)c2ccc(C(=O)NCc3nn[nH]n3)cc2)C1=O] mixture of diastereomers |
| 9.21 | 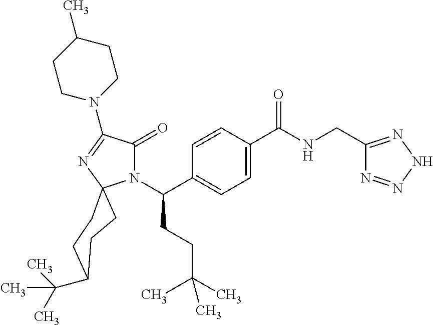 [SMILES: CC1CCN(C2=NC3(CCC(C(C)(C)C)CC3)N([C@H](CCC(C)(C)C)c3ccc(C(=O)NCc4nn[nH]n4)cc3)C2=O)CC1] |
| 9.22 | 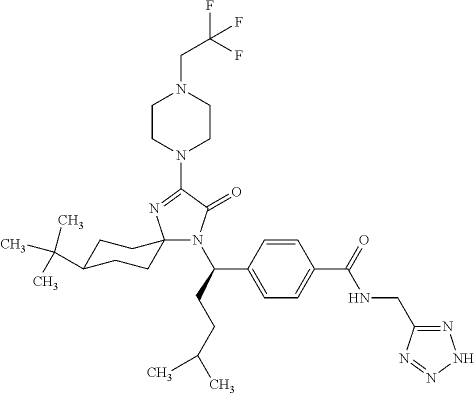 [SMILES: CC(C)CC[C@H](c1ccc(C(=O)NCc2nn[nH]n2)cc1)N1C(=O)C(N2CCN(CC(F)(F)F)CC2)=NC12CCC(C(C)(C)C)CC2] |
| 9.23 | 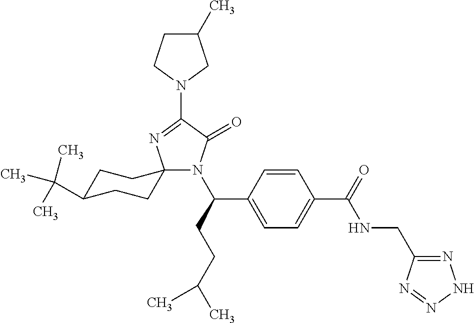 [SMILES: CC(C)CC[C@H](c1ccc(C(=O)NCc2nn[nH]n2)cc1)N1C(=O)C(N2CCC(C)C2)=NC12CCC(C(C)(C)C)CC2] mixture of diastereomers |
| 9.24 | 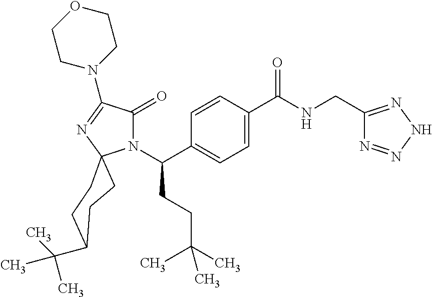 [SMILES: CC(C)(C)CC[C@H](c1ccc(C(=O)NCc2nn[nH]n2)cc1)N1C(=O)C(N2CCOCC2)=NC12CCC(C(C)(C)C)CC2] |
| 9.25 | 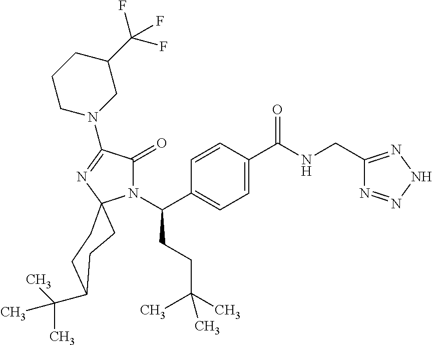 [SMILES: CC(C)(C)CC[C@H](c1ccc(C(=O)NCc2nn[nH]n2)cc1)N1C(=O)C(N2CCCC(C(F)(F)F)C2)=NC12CCC(C(C)(C)C)CC2] mixture of diastereomers |
| 9.26 | 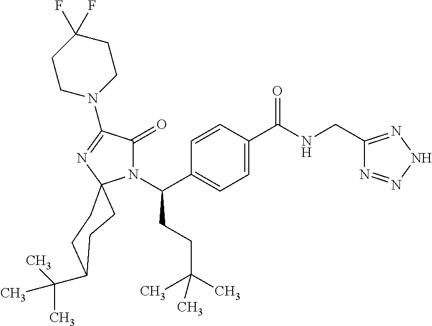 [SMILES: CC(C)(C)CC[C@H](c1ccc(C(=O)NCc2nn[nH]n2)cc1)N1C(=O)C(N2CCC(F)(F)CC2)=NC12CCC(C(C)(C)C)CC2] |
| 9.27 | 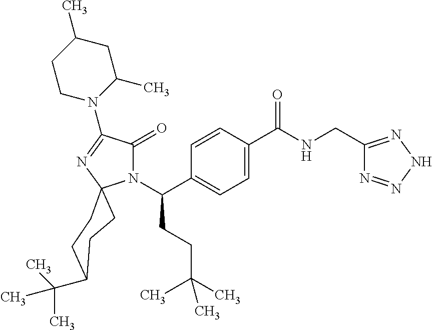 [SMILES: CC1CCN(C2=NC3(CCC(C(C)(C)C)CC3)N([C@H](CCC(C)(C)C)c3ccc(C(=O)NCc4nn[nH]n4)cc3)C2=O)C(C)C1] mixture of diastereomers |
| 9.28 | 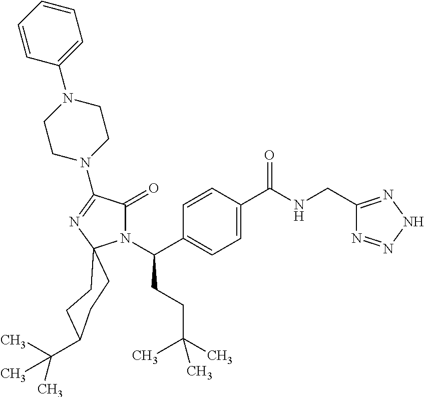 [SMILES: CC(C)(C)CC[C@H](c1ccc(C(=O)NCc2nn[nH]n2)cc1)N1C(=O)C(N2CCN(c3ccccc3)CC2)=NC12CCC(C(C)(C)C)CC2] |
| 9.29 | 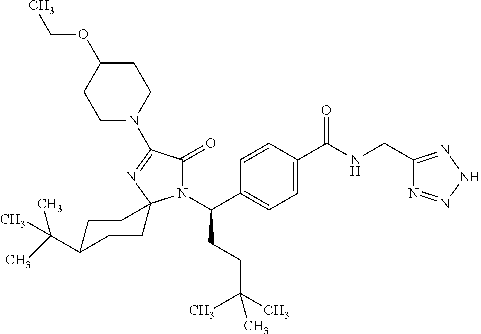 [SMILES: CCOC1CCN(C2=NC3(CCC(C(C)(C)C)CC3)N([C@H](CCC(C)(C)C)c3ccc(C(=O)NCc4nn[nH]n4)cc3)C2=O)CC1] |
| 9.30 | 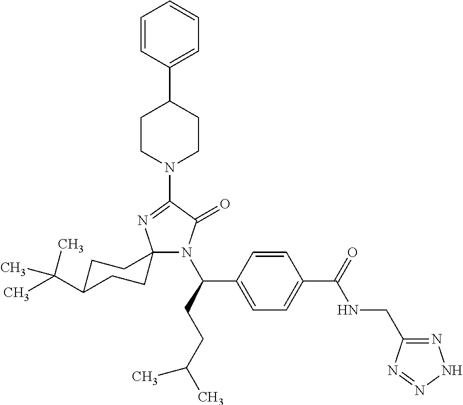 [SMILES: CC(C)CC[C@H](c1ccc(C(=O)NCc2nn[nH]n2)cc1)N1C(=O)C(N2CCC(c3ccccc3)CC2)=NC12CCC(C(C)(C)C)CC2] |
| 9.31 | 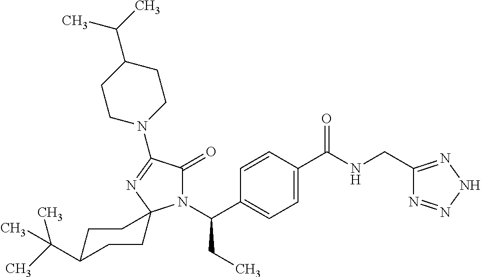 [SMILES: CC[C@H](c1ccc(C(=O)NCc2nn[nH]n2)cc1)N1C(=O)C(N2CCC(C(C)C)CC2)=NC12CCC(C(C)(C)C)CC2] |
| 9.32 | 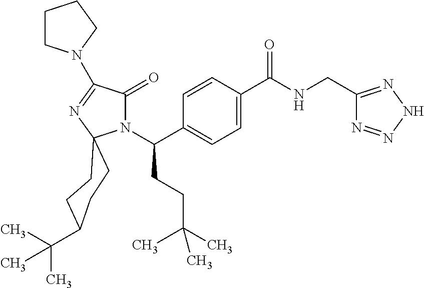 [SMILES: CC(C)(C)CC[C@H](c1ccc(C(=O)NCc2nn[nH]n2)cc1)N1C(=O)C(N2CCCC2)=NC12CCC(C(C)(C)C)CC2] |
| 9.33 | 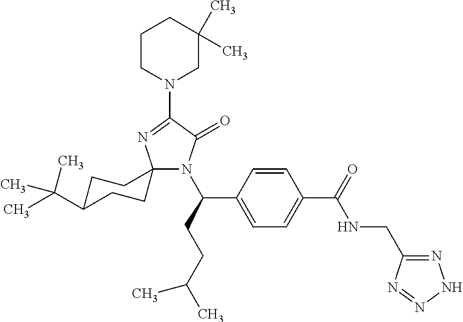 [SMILES: CC(C)CC[C@H](c1ccc(C(=O)NCc2nn[nH]n2)cc1)N1C(=O)C(N2CCCC(C)(C)C2)=NC12CCC(C(C)(C)C)CC2] |
| 9.34 | 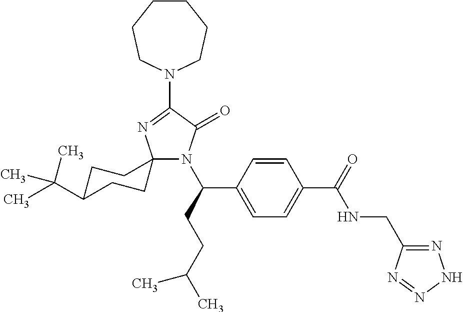 [SMILES: CC(C)CC[C@H](c1ccc(C(=O)NCc2nn[nH]n2)cc1)N1C(=O)C(N2CCCCCC2)=NC12CCC(C(C)(C)C)CC2] |
| 9.35 | 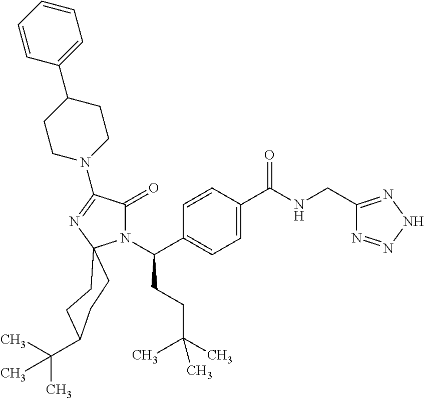 [SMILES: CC(C)(C)CC[C@H](c1ccc(C(=O)NCc2nn[nH]n2)cc1)N1C(=O)C(N2CCC(c3ccccc3)CC2)=NC12CCC(C(C)(C)C)CC2] |
| 9.36 | 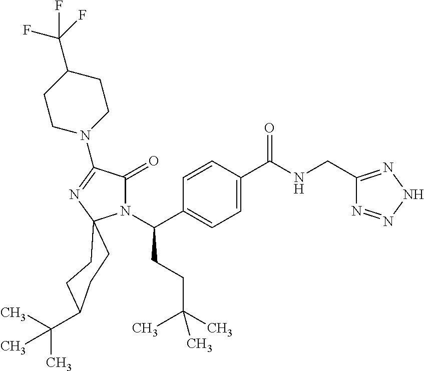 [SMILES: CC(C)(C)CC[C@H](c1ccc(C(=O)NCc2nn[nH]n2)cc1)N1C(=O)C(N2CCC(C(F)(F)F)CC2)=NC12CCC(C(C)(C)C)CC2] |
| 9.37 | 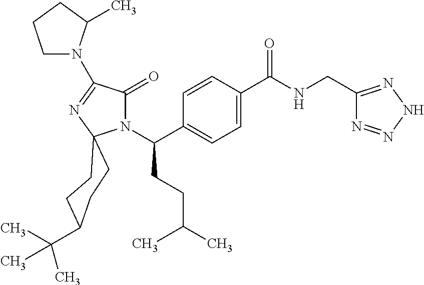 [SMILES: CC(C)CC[C@H](c1ccc(C(=O)NCc2nn[nH]n2)cc1)N1C(=O)C(N2CCCC2C)=NC12CCC(C(C)(C)C)CC2] mixture of diastereomers |
| 9.38 | 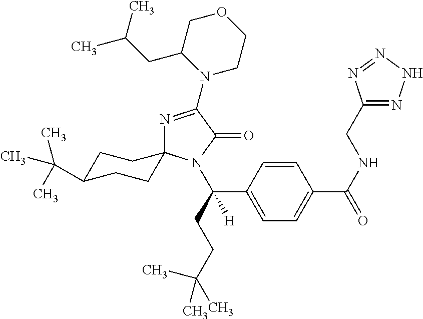 [SMILES: CC(C)CC1COCCN1C1=NC2(CCC(C(C)(C)C)CC2)N([C@H](CCC(C)(C)C)c2ccc(C(=O)NCc3nn[nH]n3)cc2)C1=O] mixture of diastereomers |
| 9.39 | 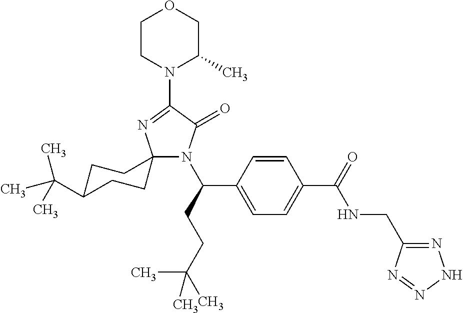 [SMILES: C[C@H]1COCCN1C1=NC2(CCC(C(C)(C)C)CC2)N([C@H](CCC(C)(C)C)c2ccc(C(=O)NCc3nn[nH]n3)cc2)C1=O] |
| 9.40 | 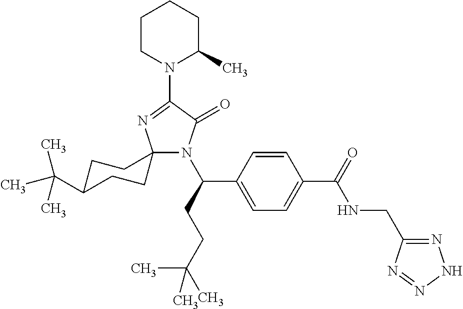 [SMILES: C[C@@H]1CCCCN1C1=NC2(CCC(C(C)(C)C)CC2)N([C@H](CCC(C)(C)C)c2ccc(C(=O)NCc3nn[nH]n3)cc2)C1=O] |
| 9.41 | 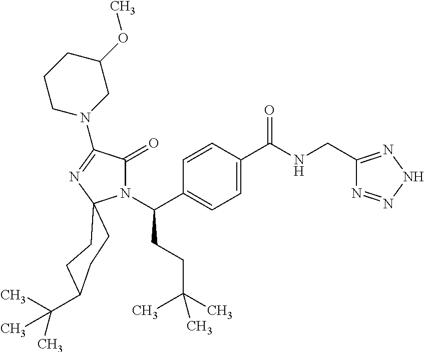 [SMILES: COC1CCCN(C2=NC3(CCC(C(C)(C)C)CC3)N([C@H](CCC(C)(C)C)c3ccc(C(=O)NCc4nn[nH]n4)cc3)C2=O)C1] mixture of diastereomers |
| 9.42 | 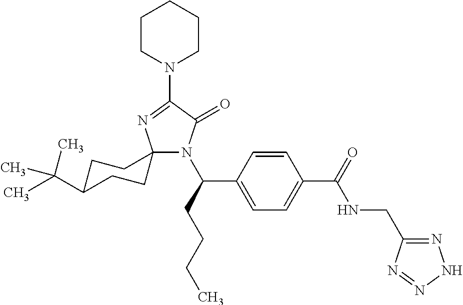 [SMILES: CCCC[C@H](c1ccc(C(=O)NCc2nn[nH]n2)cc1)N1C(=O)C(N2CCCCC2)=NC12CCC(C(C)(C)C)CC2] |
| 9.43 | 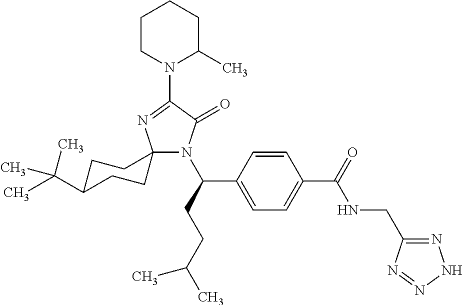 [SMILES: CC(C)CC[C@H](c1ccc(C(=O)NCc2nn[nH]n2)cc1)N1C(=O)C(N2CCCCC2C)=NC12CCC(C(C)(C)C)CC2] mixture of diastereomers |
| 9.44 | 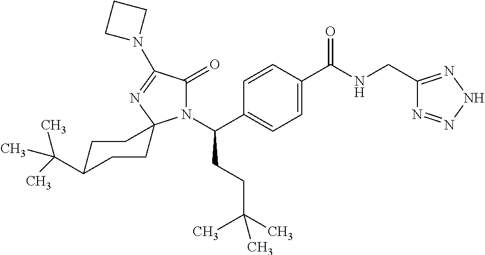 [SMILES: CC(C)(C)CC[C@H](c1ccc(C(=O)NCc2nn[nH]n2)cc1)N1C(=O)C(N2CCC2)=NC12CCC(C(C)(C)C)CC2] |
| 9.45 | 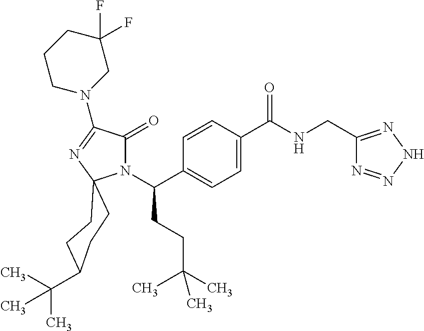 [SMILES: CC(C)(C)CC[C@H](c1ccc(C(=O)NCc2nn[nH]n2)cc1)N1C(=O)C(N2CCCC(F)(F)C2)=NC12CCC(C(C)(C)C)CC2] |
| 9.46 | 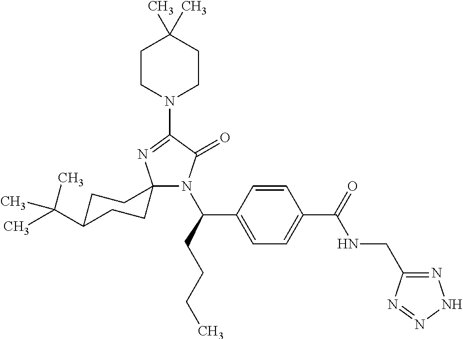 [SMILES: CCCC[C@H](c1ccc(C(=O)NCc2nn[nH]n2)cc1)N1C(=O)C(N2CCC(C)(C)CC2)=NC12CCC(C(C)(C)C)CC2] |
| 9.47 | 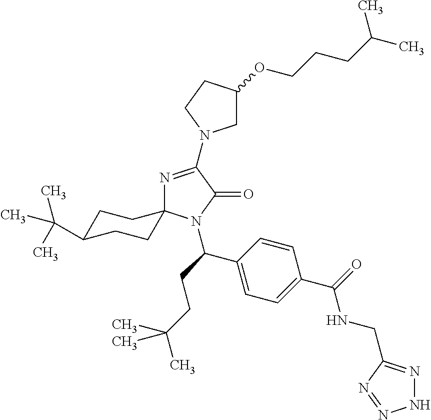 [SMILES: CC(C)CCCOC1CCN(C2=NC3(CCC(C(C)(C)C)CC3)N([C@H](CCC(C)(C)C)c3ccc(C(=O)NCc4nn[nH]n4)cc3)C2=O)C1] mixture of diastereomers |
| 9.48 | 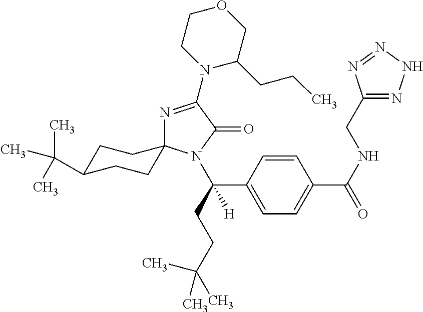 [SMILES: CCCC1COCCN1C1=NC2(CCC(C(C)(C)C)CC2)N([C@H](CCC(C)(C)C)c2ccc(C(=O)NCc3nn[nH]n3)cc2)C1=O] mixture of diastereomers |
| 9.49 | 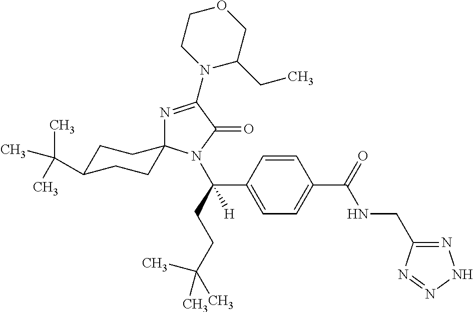 [SMILES: CCC1COCCN1C1=NC2(CCC(C(C)(C)C)CC2)N([C@H](CCC(C)(C)C)c2ccc(C(=O)NCc3nn[nH]n3)cc2)C1=O] mixture of diastereomers |
| 9.50 | 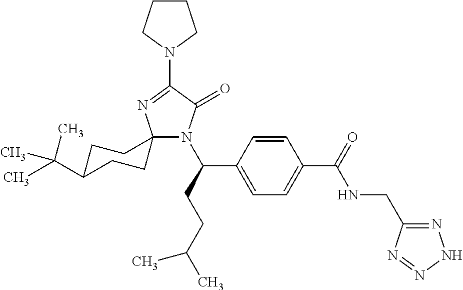 [SMILES: CC(C)CC[C@H](c1ccc(C(=O)NCc2nn[nH]n2)cc1)N1C(=O)C(N2CCCC2)=NC12CCC(C(C)(C)C)CC2] |
| 9.51 | 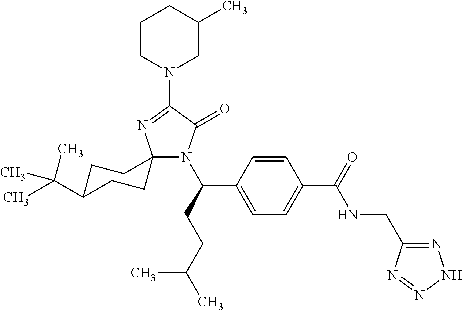 [SMILES: CC(C)CC[C@H](c1ccc(C(=O)NCc2nn[nH]n2)cc1)N1C(=O)C(N2CCCC(C)C2)=NC12CCC(C(C)(C)C)CC2] mixture of diastereomers |
| 9.52 | 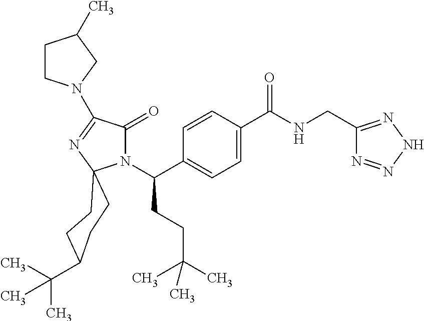 [SMILES: CC1CCN(C2=NC3(CCC(C(C)(C)C)CC3)N([C@H](CCC(C)(C)C)c3ccc(C(=O)NCc4nn[nH]n4)cc3)C2=O)C1] mixture of diastereomers |
| 9.54 | 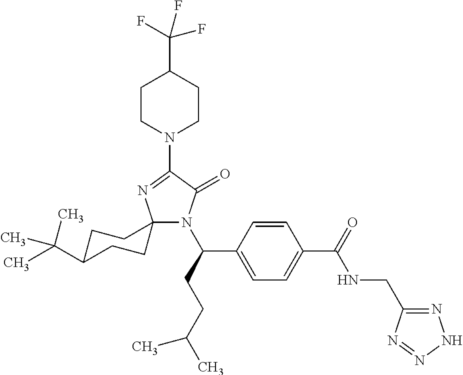 [SMILES: CC(C)CC[C@H](c1ccc(C(=O)NCc2nn[nH]n2)cc1)N1C(=O)C(N2CCC(C(F)(F)F)CC2)=NC12CCC(C(C)(C)C)CC2] |
| 9.55 | 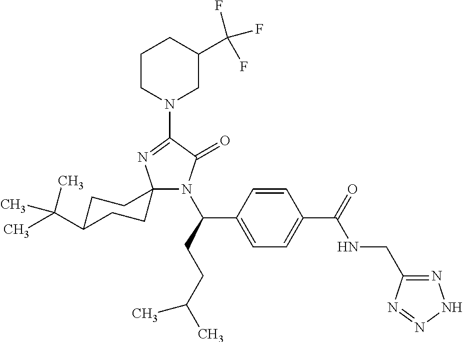 [SMILES: CC(C)CC[C@H](c1ccc(C(=O)NCc2nn[nH]n2)cc1)N1C(=O)C(N2CCCC(C(F)(F)F)C2)=NC12CCC(C(C)(C)C)CC2] mixture of diastereomers |
| 9.99 | 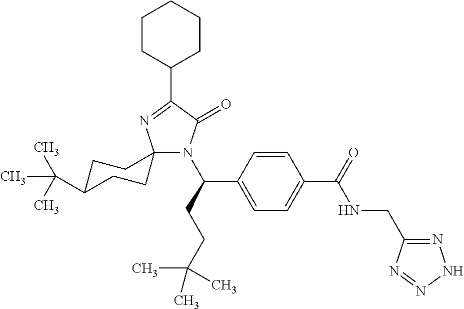 [SMILES: CC(C)(C)CC[C@H](c1ccc(C(=O)NCc2nn[nH]n2)cc1)N1C(=O)C(C2CCCCC2)=NC12CCC(C(C)(C)C)CC2] |
| 9.100 | 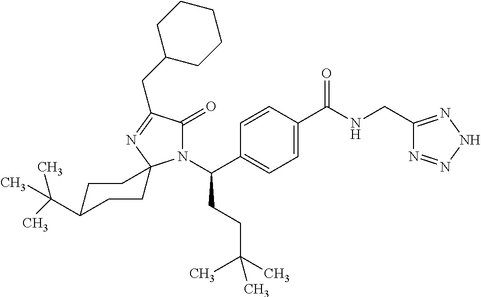 [SMILES: CC(C)(C)CC[C@H](c1ccc(C(=O)NCc2nn[nH]n2)cc1)N1C(=O)C(CC2CCCCC2)=NC12CCC(C(C)(C)C)CC2] |
| 9.101 | 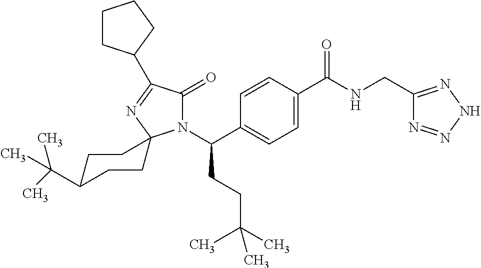 [SMILES: CC(C)(C)CC[C@H](c1ccc(C(=O)NCc2nn[nH]n2)cc1)N1C(=O)C(C2CCCC2)=NC12CCC(C(C)(C)C)CC2] |
| 9.102 | 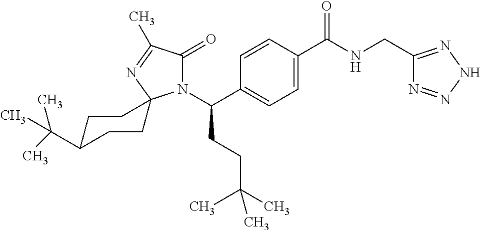 [SMILES: CC1=NC2(CCC(C(C)(C)C)CC2)N([C@H](CCC(C)(C)C)c2ccc(C(=O)NCc3nn[nH]n3)cc2)C1=O] |
| 9.103 | 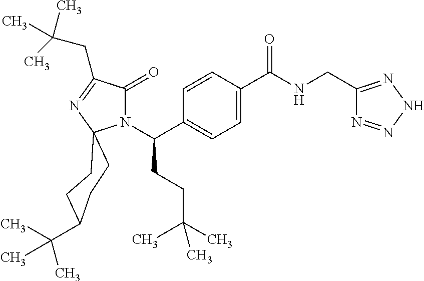 [SMILES: CC(C)(C)CC[C@H](c1ccc(C(=O)NCc2nn[nH]n2)cc1)N1C(=O)C(CC(C)(C)C)=NC12CCC(C(C)(C)C)CC2] |
| 9.104 | 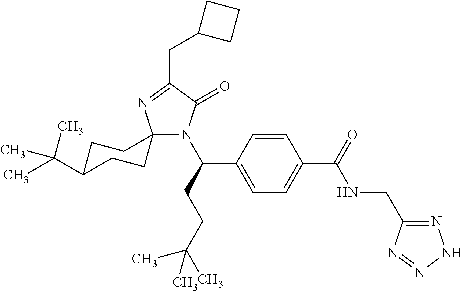 [SMILES: CC(C)(C)CC[C@H](c1ccc(C(=O)NCc2nn[nH]n2)cc1)N1C(=O)C(CC2CCC2)=NC12CCC(C(C)(C)C)CC2] |
| 9.105 | 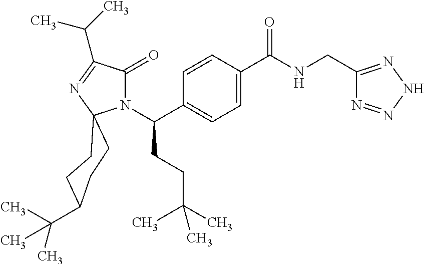 [SMILES: CC(C)C1=NC2(CCC(C(C)(C)C)CC2)N([C@H](CCC(C)(C)C)c2ccc(C(=O)NCc3nn[nH]n3)cc2)C1=O] |
| 9.106 | 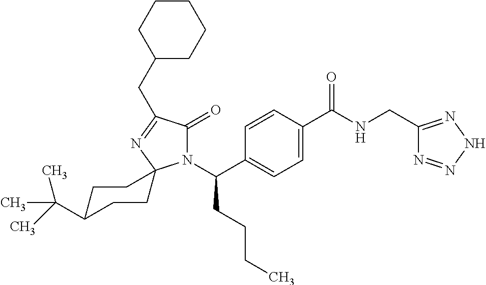 [SMILES: CCCC[C@H](c1ccc(C(=O)NCc2nn[nH]n2)cc1)N1C(=O)C(CC2CCCCC2)=NC12CCC(C(C)(C)C)CC2] |
| 9.118 | 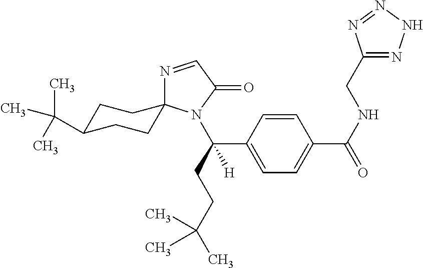 [SMILES: CC(C)(C)CC[C@H](c1ccc(C(=O)NCc2nn[nH]n2)cc1)N1C(=O)C=NC12CCC(C(C)(C)C)CC2] |
| 9.119 | 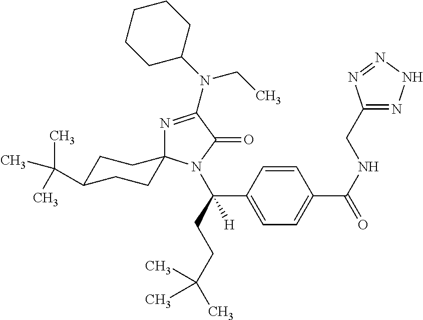 [SMILES: CCN(C1=NC2(CCC(C(C)(C)C)CC2)N([C@H](CCC(C)(C)C)c2ccc(C(=O)NCc3nn[nH]n3)cc2)C1=O)C1CCCCC1] |
| 9.120 | 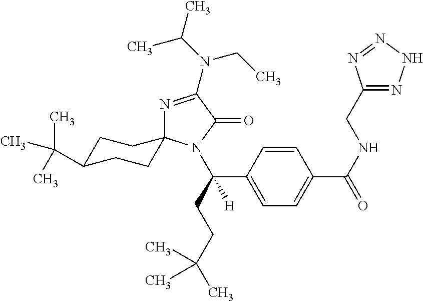 [SMILES: CCN(C1=NC2(CCC(C(C)(C)C)CC2)N([C@H](CCC(C)(C)C)c2ccc(C(=O)NCc3nn[nH]n3)cc2)C1=O)C(C)C] |
| 10.1 | 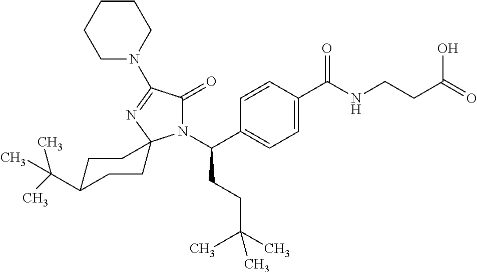 [SMILES: CC(C)(C)CC[C@H](c1ccc(C(=O)NCCC(=O)O)cc1)N1C(=O)C(N2CCCCC2)=NC12CCC(C(C)(C)C)CC2] |
| 10.2 | 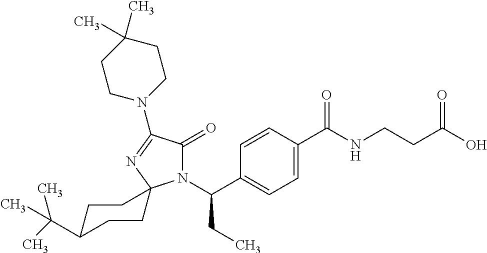 [SMILES: CC[C@H](c1ccc(C(=O)NCCC(=O)O)cc1)N1C(=O)C(N2CCC(C)(C)CC2)=NC12CCC(C(C)(C)C)CC2] |
| 10.3 | 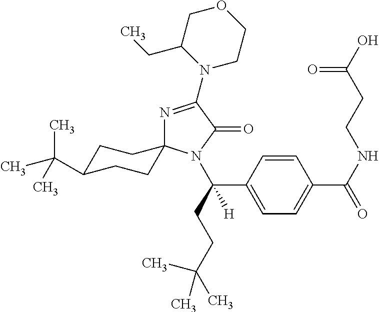 [SMILES: CCC1COCCN1C1=NC2(CCC(C(C)(C)C)CC2)N([C@H](CCC(C)(C)C)c2ccc(C(=O)NCCC(=O)O)cc2)C1=O] mixture of diastereomers |
| 10.4 | 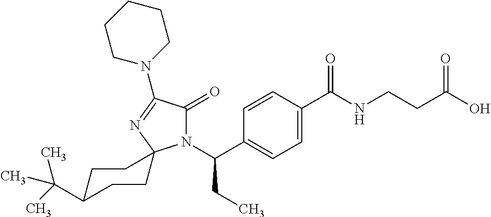 [SMILES: CC[C@H](c1ccc(C(=O)NCCC(=O)O)cc1)N1C(=O)C(N2CCCCC2)=NC12CCC(C(C)(C)C)CC2] |
| 10.5 | 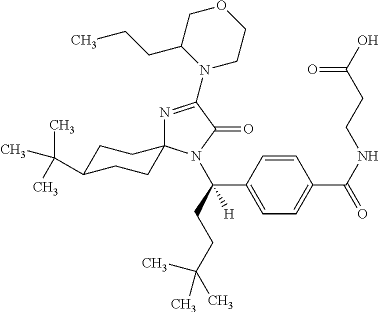 [SMILES: CCCC1COCCN1C1=NC2(CCC(C(C)(C)C)CC2)N([C@H](CCC(C)(C)C)c2ccc(C(=O)NCCC(=O)O)cc2)C1=O] mixture of diastereomers |
| 10.6 | 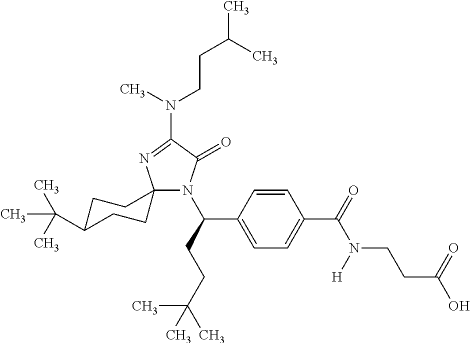 [SMILES: CC(C)CCN(C)C1=NC2(CCC(C(C)(C)C)CC2)N([C@H](CCC(C)(C)C)c2ccc(C(=O)NCCC(=O)O)cc2)C1=O] |
| 10.7 | 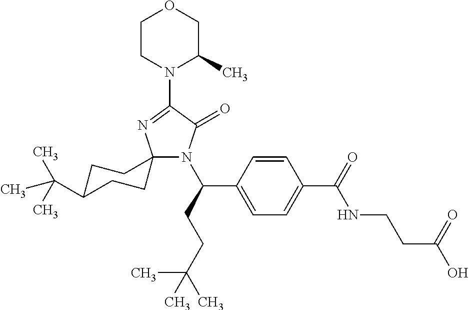 [SMILES: C[C@@H]1COCCN1C1=NC2(CCC(C(C)(C)C)CC2)N([C@H](CCC(C)(C)C)c2ccc(C(=O)NCCC(=O)O)cc2)C1=O] |
| 10.8 | 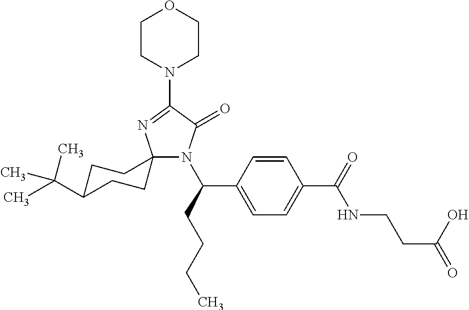 [SMILES: CCCC[C@H](c1ccc(C(=O)NCCC(=O)O)cc1)N1C(=O)C(N2CCOCC2)=NC12CCC(C(C)(C)C)CC2] |
| 10.9 | 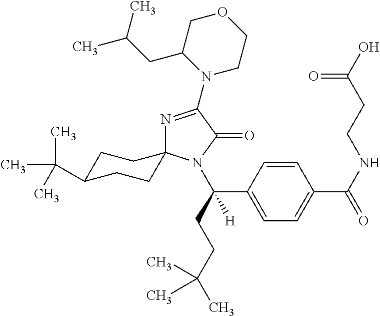 [SMILES: CC(C)CC1COCCN1C1=NC2(CCC(C(C)(C)C)CC2)N([C@H](CCC(C)(C)C)c2ccc(C(=O)NCCC(=O)O)cc2)C1=O] mixture of diastereomers |
| 10.10 | 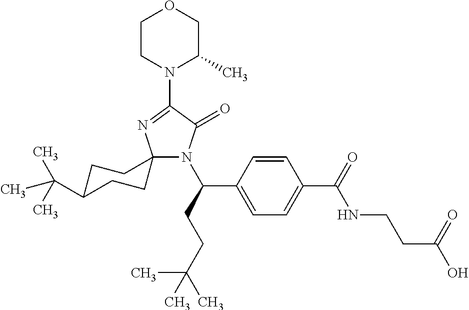 [SMILES: C[C@H]1COCCN1C1=NC2(CCC(C(C)(C)C)CC2)N([C@H](CCC(C)(C)C)c2ccc(C(=O)NCCC(=O)O)cc2)C1=O] |
| 10.11 | 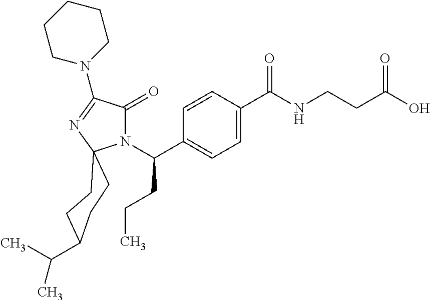 [SMILES: CCC[C@H](c1ccc(C(=O)NCCC(=O)O)cc1)N1C(=O)C(N2CCCCC2)=NC12CCC(C(C)C)CC2] |
| 10.12 | 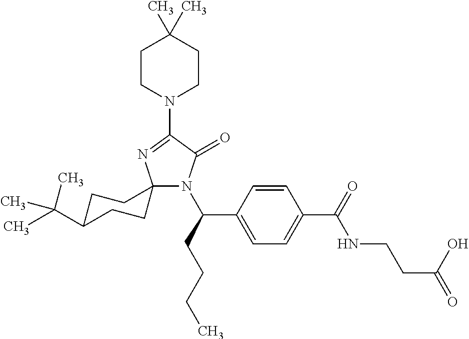 [SMILES: CCCC[C@H](c1ccc(C(=O)NCCC(=O)O)cc1)N1C(=O)C(N2CCC(C)(C)CC2)=NC12CCC(C(C)(C)C)CC2] |
| 10.13 | 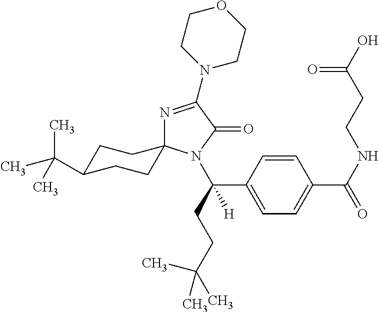 [SMILES: CC(C)(C)CC[C@H](c1ccc(C(=O)NCCC(=O)O)cc1)N1C(=O)C(N2CCOCC2)=NC12CCC(C(C)(C)C)CC2] |
| 10.14 | 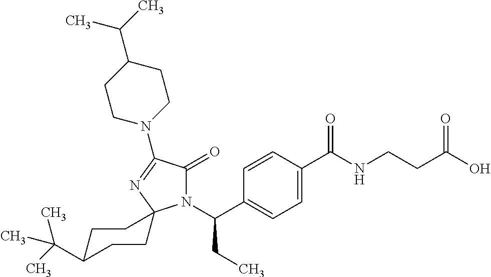 [SMILES: CC[C@H](c1ccc(C(=O)NCCC(=O)O)cc1)N1C(=O)C(N2CCC(C(C)C)CC2)=NC12CCC(C(C)(C)C)CC2] |
| 10.15 | 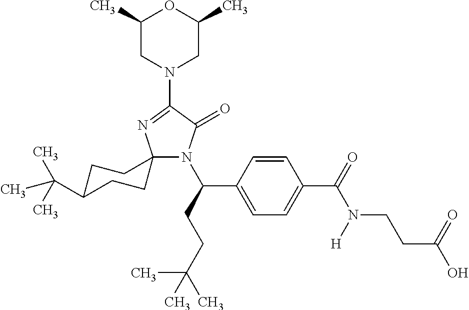 [SMILES: C[C@@H]1CN(C2=NC3(CCC(C(C)(C)C)CC3)N([C@H](CCC(C)(C)C)c3ccc(C(=O)NCCC(=O)O)cc3)C2=O)C[C@H](C)O1] |
| 10.22 | 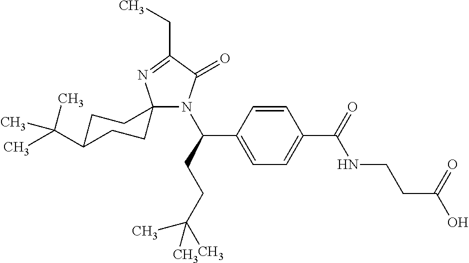 [SMILES: CCC1=NC2(CCC(C(C)(C)C)CC2)N([C@H](CCC(C)(C)C)c2ccc(C(=O)NCCC(=O)O)cc2)C1=O] |
| 10.23 | 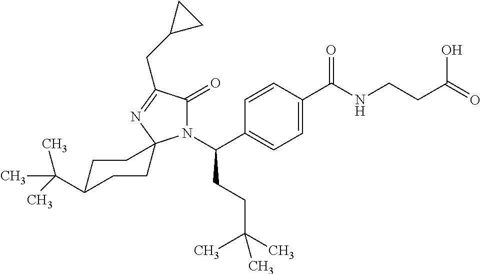 [SMILES: CC(C)(C)CC[C@H](c1ccc(C(=O)NCCC(=O)O)cc1)N1C(=O)C(CC2CC2)=NC12CCC(C(C)(C)C)CC2] |
| 10.24 | 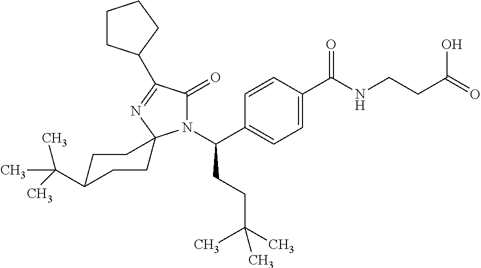 [SMILES: CC(C)(C)CC[C@H](c1ccc(C(=O)NCCC(=O)O)cc1)N1C(=O)C(C2CCCC2)=NC12CCC(C(C)(C)C)CC2] |
| 10.25 | 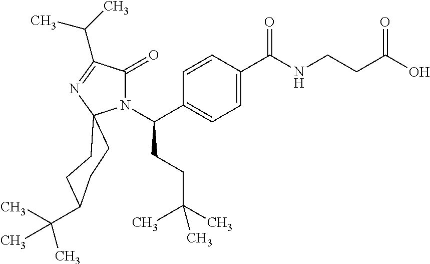 [SMILES: CC(C)C1=NC2(CCC(C(C)(C)C)CC2)N([C@H](CCC(C)(C)C)c2ccc(C(=O)NCCC(=O)O)cc2)C1=O] |
| 10.26 | 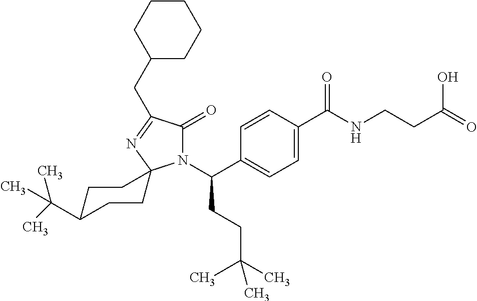 [SMILES: CC(C)(C)CC[C@H](c1ccc(C(=O)NCCC(=O)O)cc1)N1C(=O)C(CC2CCCCC2)=NC12CCC(C(C)(C)C)CC2] |
| 10.27 | 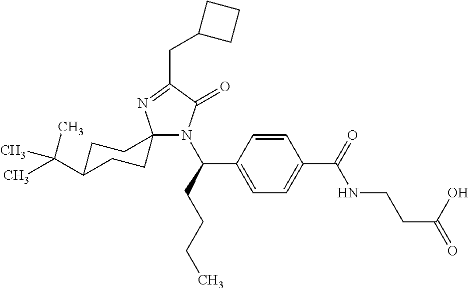 [SMILES: CCCC[C@H](c1ccc(C(=O)NCCC(=O)O)cc1)N1C(=O)C(CC2CCC2)=NC12CCC(C(C)(C)C)CC2] |
| 10.28 | 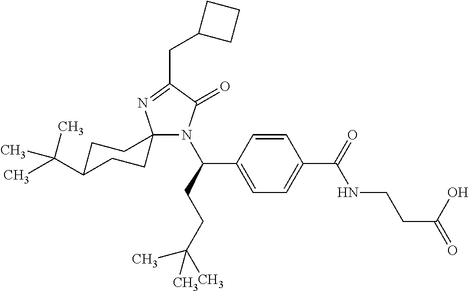 [SMILES: CC(C)(C)CC[C@H](c1ccc(C(=O)NCCC(=O)O)cc1)N1C(=O)C(CC2CCC2)=NC12CCC(C(C)(C)C)CC2] |
| 10.29 | 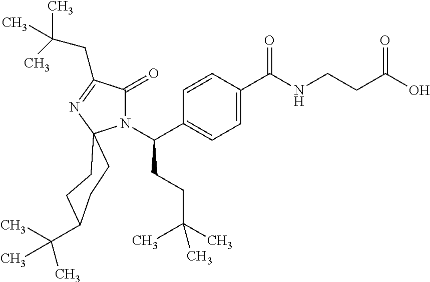 [SMILES: CC(C)(C)CC[C@H](c1ccc(C(=O)NCCC(=O)O)cc1)N1C(=O)C(CC(C)(C)C)=NC12CCC(C(C)(C)C)CC2] |
| 10.30 | 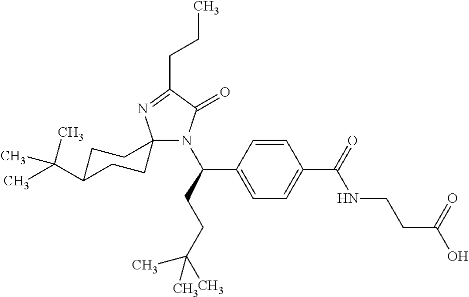 [SMILES: CCCC1=NC2(CCC(C(C)(C)C)CC2)N([C@H](CCC(C)(C)C)c2ccc(C(=O)NCCC(=O)O)cc2)C1=O] |
| 10.31 | 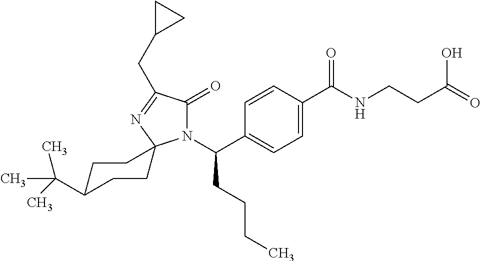 [SMILES: CCCC[C@H](c1ccc(C(=O)NCCC(=O)O)cc1)N1C(=O)C(CC2CC2)=NC12CCC(C(C)(C)C)CC2] |
| 10.32 | 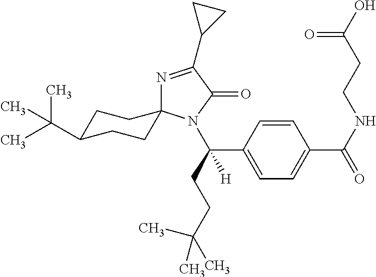 [SMILES: CC(C)(C)CC[C@H](c1ccc(C(=O)NCCC(=O)O)cc1)N1C(=O)C(C2CC2)=NC12CCC(C(C)(C)C)CC2] |
| 10.33 | 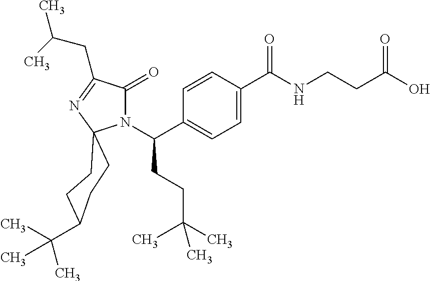 [SMILES: CC(C)CC1=NC2(CCC(C(C)(C)C)CC2)N([C@H](CCC(C)(C)C)c2ccc(C(=O)NCCC(=O)O)cc2)C1=O] |
| 10.34 | 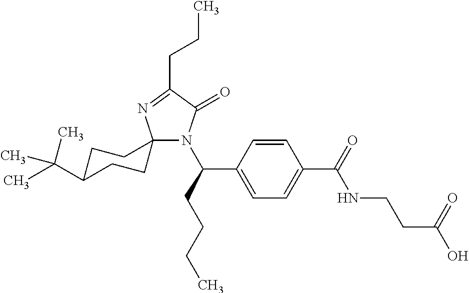 [SMILES: CCCC[C@H](c1ccc(C(=O)NCCC(=O)O)cc1)N1C(=O)C(CCC)=NC12CCC(C(C)(C)C)CC2] |
| 10.35 | 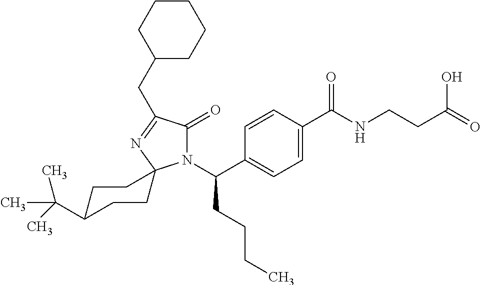 [SMILES: CCCC[C@H](c1ccc(C(=O)NCCC(=O)O)cc1)N1C(=O)C(CC2CCCCC2)=NC12CCC(C(C)(C)C)CC2] |
| 10.36 | 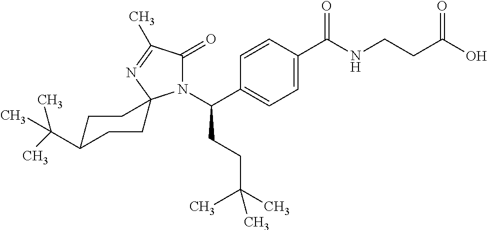 [SMILES: CC1=NC2(CCC(C(C)(C)C)CC2)N([C@H](CCC(C)(C)C)c2ccc(C(=O)NCCC(=O)O)cc2)C1=O] |
| 10.41 | 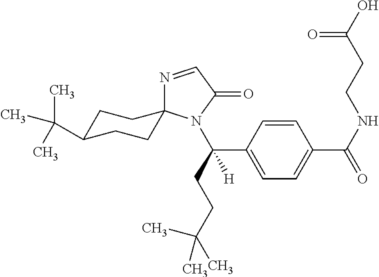 [SMILES: CC(C)(C)CC[C@H](c1ccc(C(=O)NCCC(=O)O)cc1)N1C(=O)C=NC12CCC(C(C)(C)C)CC2] |
| 10.42 | 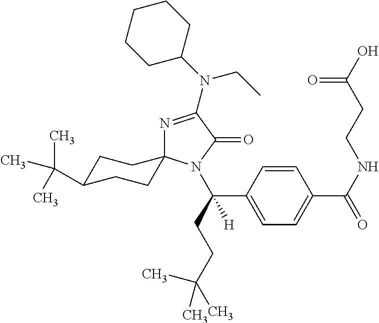 [SMILES: CCN(C1=NC2(CCC(C(C)(C)C)CC2)N([C@H](CCC(C)(C)C)c2ccc(C(=O)NCCC(=O)O)cc2)C1=O)C1CCCCC1] |
| 10.43 | 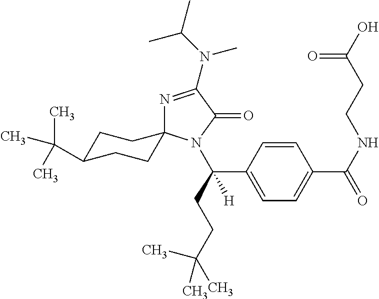 [SMILES: CC(C)N(C)C1=NC2(CCC(C(C)(C)C)CC2)N([C@H](CCC(C)(C)C)c2ccc(C(=O)NCCC(=O)O)cc2)C1=O] |
| 10.45 | 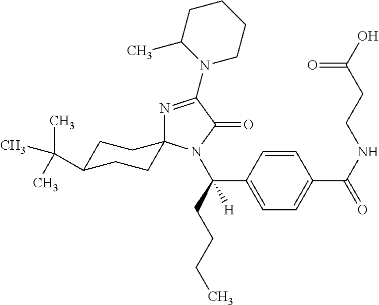 [SMILES: CCCC[C@H](c1ccc(C(=O)NCCC(=O)O)cc1)N1C(=O)C(N2CCCCC2C)=NC12CCC(C(C)(C)C)CC2] mixture of diastereomers |
| 10.46 | 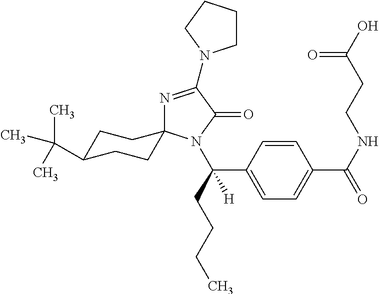 [SMILES: CCCC[C@H](c1ccc(C(=O)NCCC(=O)O)cc1)N1C(=O)C(N2CCCC2)=NC12CCC(C(C)(C)C)CC2] |
| 10.48 | 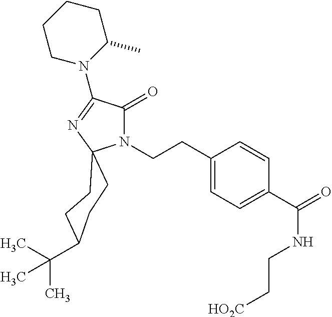 [SMILES: C[C@H]1CCCCN1C1=NC2(CCC(C(C)(C)C)CC2)N(CCc2ccc(C(=O)NCCC(=O)O)cc2)C1=O] |
| 10.49 | 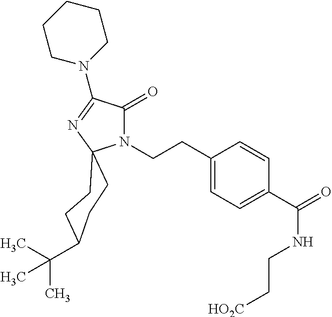 [SMILES: CC(C)(C)C1CCC2(CC1)N=C(N1CCCCC1)C(=O)N2CCc1ccc(C(=O)NCCC(=O)O)cc1] |

15. A compound of claim 1, or a pharmaceutically acceptable salt or tautomer of said compound, said compound selected from the group consisting of:

| Ex. | Structure |
|---|---|
| 9.1 | 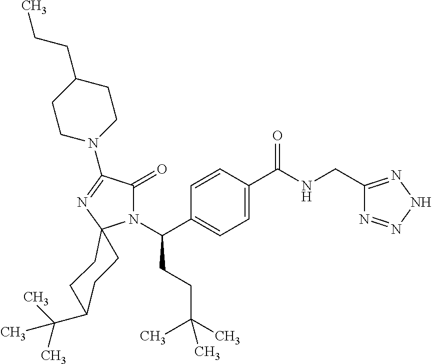 [SMILES: CCCC1CCN(C2=NC3(CCC(C(C)(C)C)CC3)N([C@H](CCC(C)(C)C)c3ccc(C(=O)NCc4nn[nH]n4)cc3)C2=O)CC1] |
| 9.2 | 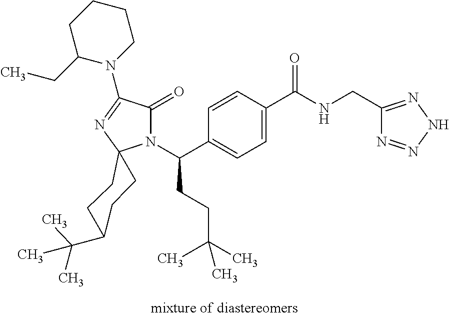 [SMILES: CCC1CCCCN1C1=NC2(CCC(C(C)(C)C)CC2)N([C@H](CCC(C)(C)C)c2ccc(C(=O)NCc3nn[nH]n3)cc2)C1=O] |
| 9.3 | 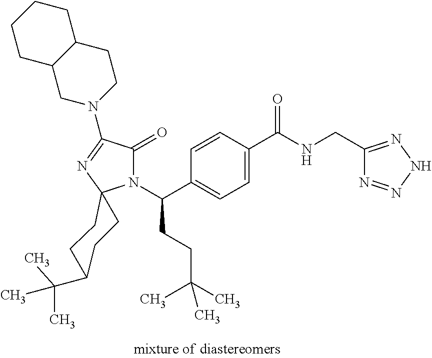 [SMILES: CC(C)(C)CC[C@H](c1ccc(C(=O)NCc2nn[nH]n2)cc1)N1C(=O)C(N2CCC3CCCCC3C2)=NC12CCC(C(C)(C)C)CC2] |
| 9.4 | 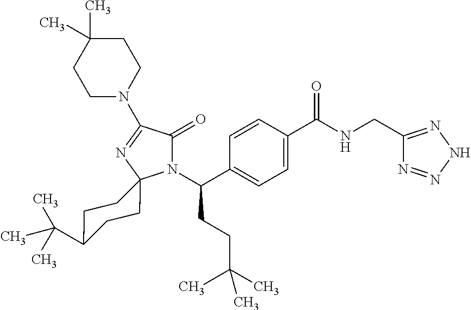 [SMILES: CC(C)(C)CC[C@H](c1ccc(C(=O)NCc2nn[nH]n2)cc1)N1C(=O)C(N2CCC(C)(C)CC2)=NC12CCC(C(C)(C)C)CC2] |
| 9.5 | 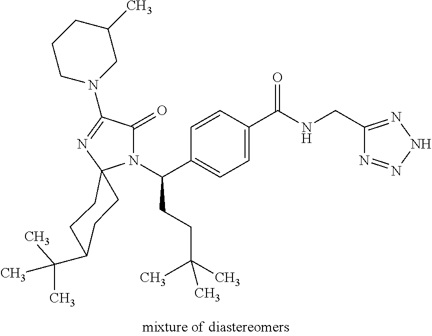 [SMILES: CC1CCCN(C2=NC3(CCC(C(C)(C)C)CC3)N([C@H](CCC(C)(C)C)c3ccc(C(=O)NCc4nn[nH]n4)cc3)C2=O)C1] |
| 9.6 | 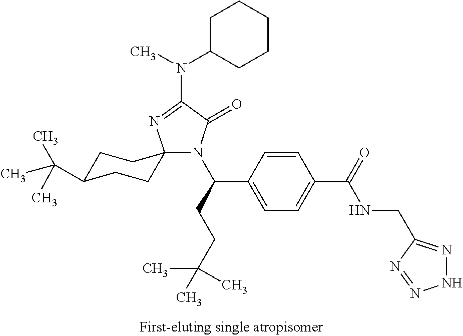 [SMILES: CN(C1=NC2(CCC(C(C)(C)C)CC2)N([C@H](CCC(C)(C)C)c2ccc(C(=O)NCc3nn[nH]n3)cc2)C1=O)C1CCCCC1] |
| 9.7 | 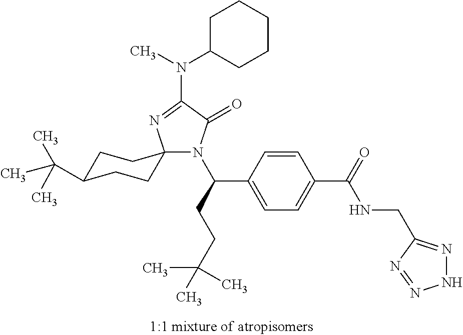 [SMILES: CN(C1=NC2(CCC(C(C)(C)C)CC2)N([C@H](CCC(C)(C)C)c2ccc(C(=O)NCc3nn[nH]n3)cc2)C1=O)C1CCCCC1] |
| 9.8 | 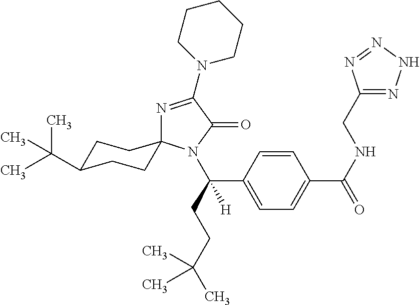 [SMILES: CC(C)(C)CC[C@H](c1ccc(C(=O)NCc2nn[nH]n2)cc1)N1C(=O)C(N2CCCCC2)=NC12CCC(C(C)(C)C)CC2] |
| 9.9 | 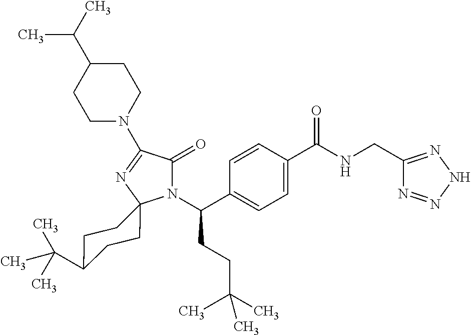 [SMILES: CC(C)C1CCN(C2=NC3(CCC(C(C)(C)C)CC3)N([C@H](CCC(C)(C)C)c3ccc(C(=O)NCc4nn[nH]n4)cc3)C2=O)CC1] |
| 9.10 | 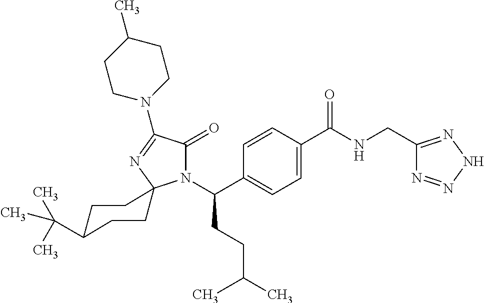 [SMILES: CC(C)CC[C@H](c1ccc(C(=O)NCc2nn[nH]n2)cc1)N1C(=O)C(N2CCC(C)CC2)=NC12CCC(C(C)(C)C)CC2] |
| 9.11 | 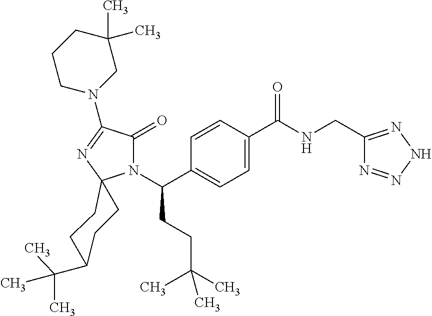 [SMILES: CC(C)(C)CC[C@H](c1ccc(C(=O)NCc2nn[nH]n2)cc1)N1C(=O)C(N2CCCC(C)(C)C2)=NC12CCC(C(C)(C)C)CC2] |
| 9.12 | 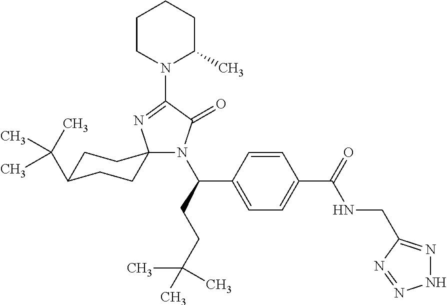 [SMILES: C[C@H]1CCCCN1C1=NC2(CCC(C(C)(C)C)CC2)N([C@H](CCC(C)(C)C)c2ccc(C(=O)NCc3nn[nH]n3)cc2)C1=O] |
| 9.13 | 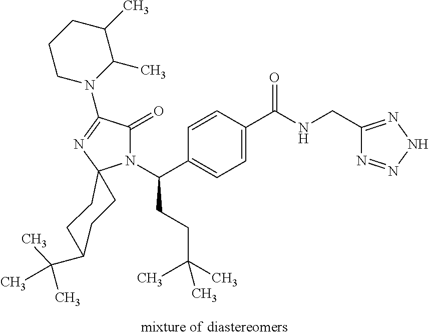 [SMILES: CC1CCCN(C2=NC3(CCC(C(C)(C)C)CC3)N([C@H](CCC(C)(C)C)c3ccc(C(=O)NCc4nn[nH]n4)cc3)C2=O)C1C] |
| 9.14 | 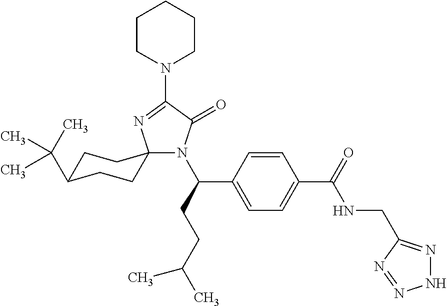 [SMILES: CC(C)CC[C@H](c1ccc(C(=O)NCc2nn[nH]n2)cc1)N1C(=O)C(N2CCCCC2)=NC12CCC(C(C)(C)C)CC2] |
| 9.15 | 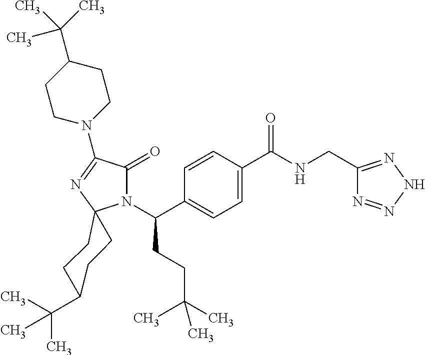 [SMILES: CC(C)(C)CC[C@H](c1ccc(C(=O)NCc2nn[nH]n2)cc1)N1C(=O)C(N2CCC(C(C)(C)C)CC2)=NC12CCC(C(C)(C)C)CC2] |
| 9.16 | 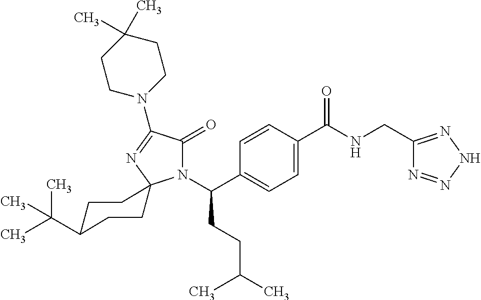 [SMILES: CC(C)CC[C@H](c1ccc(C(=O)NCc2nn[nH]n2)cc1)N1C(=O)C(N2CCC(C)(C)CC2)=NC12CCC(C(C)(C)C)CC2] |
| 9.17 | 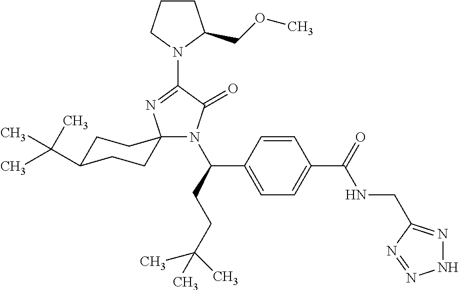 [SMILES: COC[C@@H]1CCCN1C1=NC2(CCC(C(C)(C)C)CC2)N([C@H](CCC(C)(C)C)c2ccc(C(=O)NCc3nn[nH]n3)cc2)C1=O] |
| 9.18 | 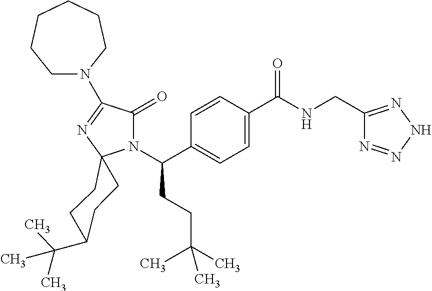 [SMILES: CC(C)(C)CC[C@H](c1ccc(C(=O)NCc2nn[nH]n2)cc1)N1C(=O)C(N2CCCCCC2)=NC12CCC(C(C)(C)C)CC2] |
| 9.19 | 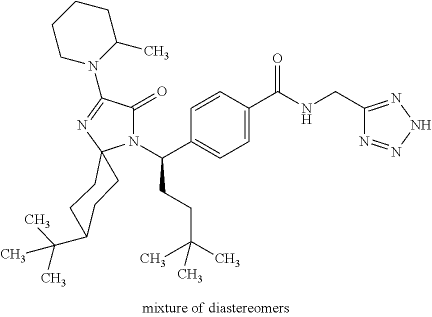 [SMILES: CC1CCCCN1C1=NC2(CCC(C(C)(C)C)CC2)N([C@H](CCC(C)(C)C)c2ccc(C(=O)NCc3nn[nH]n3)cc2)C1=O] |
| 9.20 | 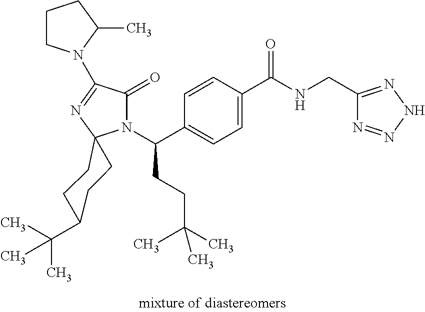 [SMILES: CC1CCCN1C1=NC2(CCC(C(C)(C)C)CC2)N([C@H](CCC(C)(C)C)c2ccc(C(=O)NCc3nn[nH]n3)cc2)C1=O] |
| 9.21 | 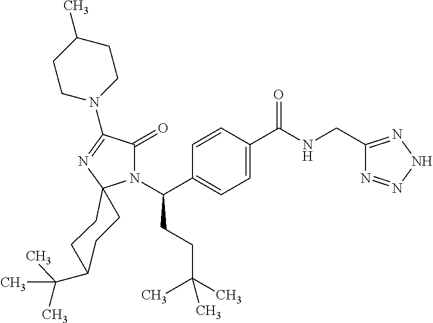 [SMILES: CC1CCN(C2=NC3(CCC(C(C)(C)C)CC3)N([C@H](CCC(C)(C)C)c3ccc(C(=O)NCc4nn[nH]n4)cc3)C2=O)CC1] |
| 9.22 | 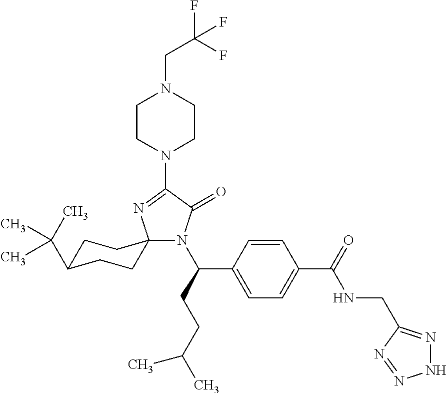 [SMILES: CC(C)CC[C@H](c1ccc(C(=O)NCc2nn[nH]n2)cc1)N1C(=O)C(N2CCN(CC(F)(F)F)CC2)=NC12CCC(C(C)(C)C)CC2] |
| 9.23 | 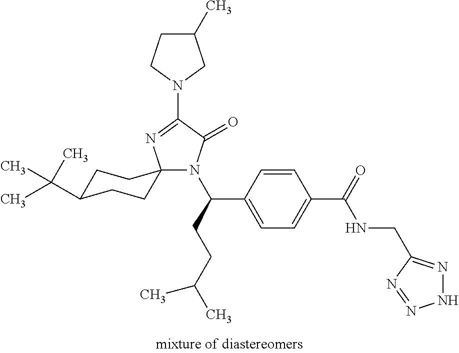 [SMILES: CC(C)CC[C@H](c1ccc(C(=O)NCc2nn[nH]n2)cc1)N1C(=O)C(N2CCC(C)C2)=NC12CCC(C(C)(C)C)CC2] |
| 9.24 | 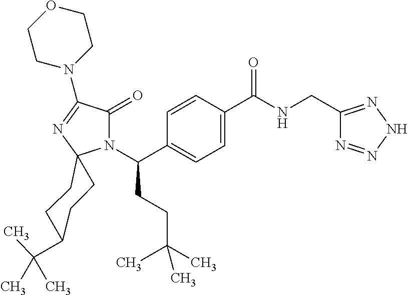 [SMILES: CC(C)(C)CC[C@H](c1ccc(C(=O)NCc2nn[nH]n2)cc1)N1C(=O)C(N2CCOCC2)=NC12CCC(C(C)(C)C)CC2] |
| 9.99 | 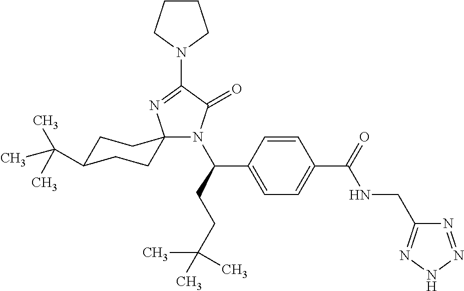 [SMILES: CC(C)(C)CC[C@H](c1ccc(C(=O)NCc2nn[nH]n2)cc1)N1C(=O)C(N2CCCC2)=NC12CCC(C(C)(C)C)CC2] |
| 9.100 | 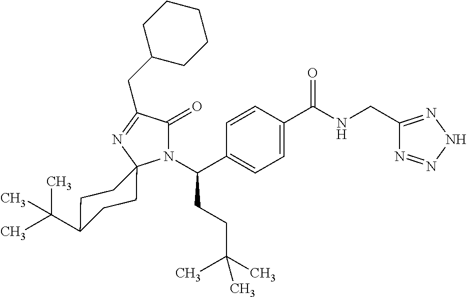 [SMILES: CC(C)(C)CC[C@H](c1ccc(C(=O)NCc2nn[nH]n2)cc1)N1C(=O)C(CC2CCCCC2)=NC12CCC(C(C)(C)C)CC2] |
| 9.101 | 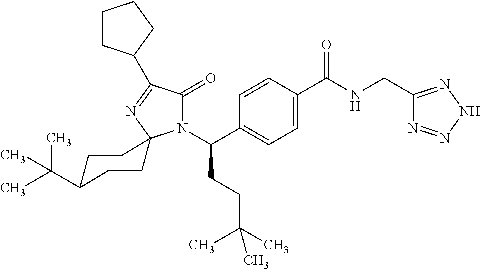 [SMILES: CC(C)(C)CC[C@H](c1ccc(C(=O)NCc2nn[nH]n2)cc1)N1C(=O)C(C2CCCC2)=NC12CCC(C(C)(C)C)CC2] |
| 9.119 | 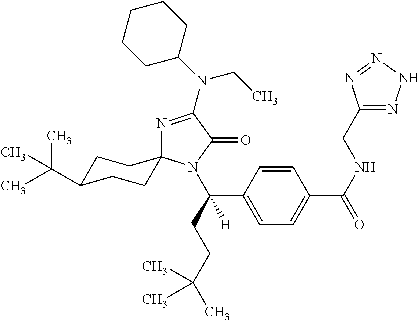 [SMILES: CCN(C1=NC2(CCC(C(C)(C)C)CC2)N([C@H](CCC(C)(C)C)c2ccc(C(=O)NCc3nn[nH]n3)cc2)C1=O)C1CCCCC1] |
| 10.1 | 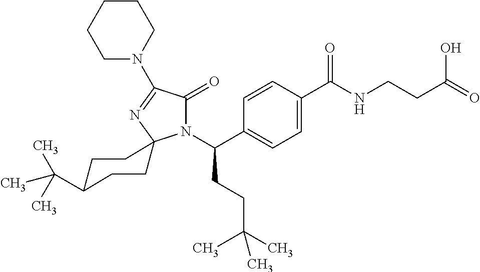 [SMILES: CC(C)(C)CC[C@H](c1ccc(C(=O)NCCC(=O)O)cc1)N1C(=O)C(N2CCCCC2)=NC12CCC(C(C)(C)C)CC2] |
| 10.2 | 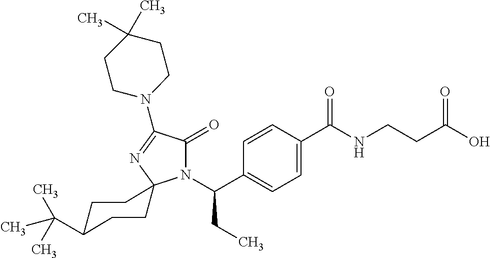 [SMILES: CC[C@H](c1ccc(C(=O)NCCC(=O)O)cc1)N1C(=O)C(N2CCC(C)(C)CC2)=NC12CCC(C(C)(C)C)CC2] |
| 10.3 | 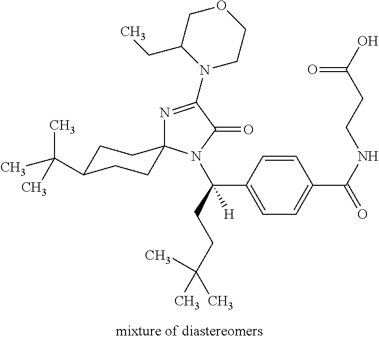 [SMILES: CCC1COCCN1C1=NC2(CCC(C(C)(C)C)CC2)N([C@H](CCC(C)(C)C)c2ccc(C(=O)NCCC(=O)O)cc2)C1=O] |
| 10.4 | 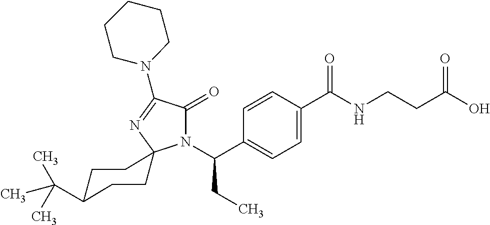 [SMILES: CC[C@H](c1ccc(C(=O)NCCC(=O)O)cc1)N1C(=O)C(N2CCCCC2)=NC12CCC(C(C)(C)C)CC2] |
| 10.5 | 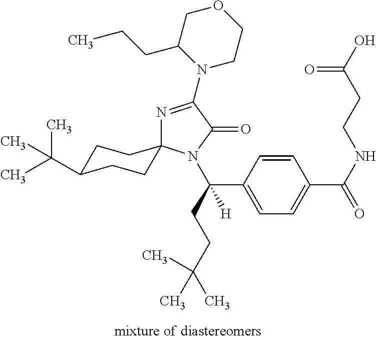 [SMILES: CCCC1COCCN1C1=NC2(CCC(C(C)(C)C)CC2)N([C@H](CCC(C)(C)C)c2ccc(C(=O)NCCC(=O)O)cc2)C1=O] |
| 10.6 | 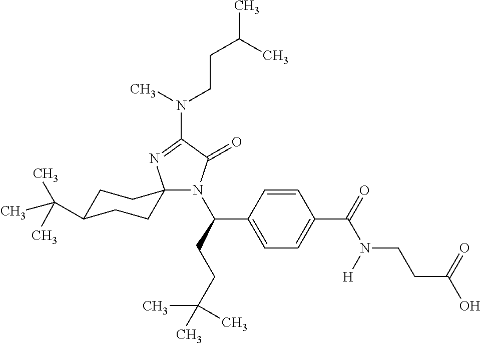 [SMILES: CC(C)CCN(C)C1=NC2(CCC(C(C)(C)C)CC2)N([C@H](CCC(C)(C)C)c2ccc(C(=O)NCCC(=O)O)cc2)C1=O] |
| 10.7 | 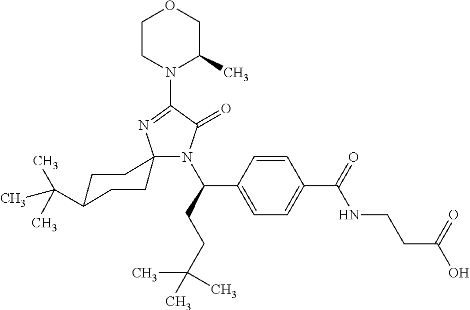 [SMILES: C[C@@H]1COCCN1C1=NC2(CCC(C(C)(C)C)CC2)N([C@H](CCC(C)(C)C)c2ccc(C(=O)NCCC(=O)O)cc2)C1=O] |
| 10.8 | 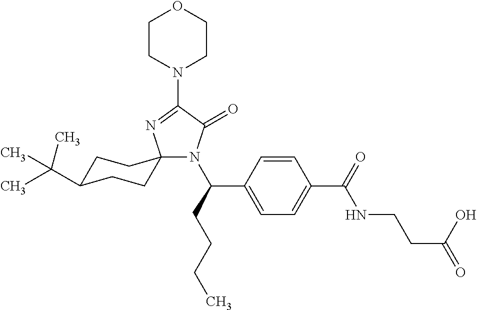 [SMILES: CCCC[C@H](c1ccc(C(=O)NCCC(=O)O)cc1)N1C(=O)C(N2CCOCC2)=NC12CCC(C(C)(C)C)CC2] |
| 10.9 | 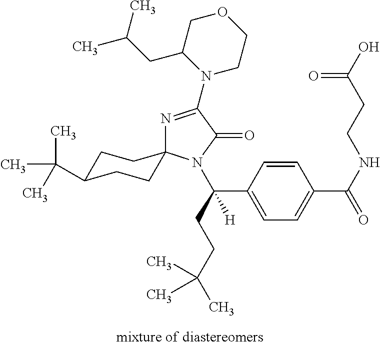 [SMILES: CC(C)CC1COCCN1C1=NC2(CCC(C(C)(C)C)CC2)N([C@H](CCC(C)(C)C)c2ccc(C(=O)NCCC(=O)O)cc2)C1=O] |
| 10.10 | 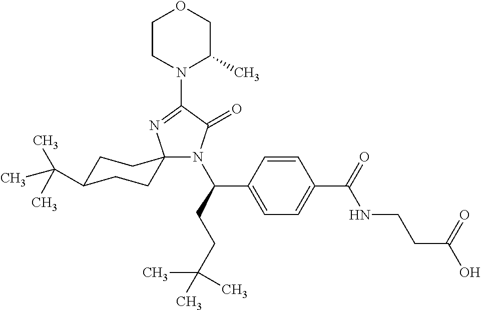 [SMILES: C[C@H]1COCCN1C1=NC2(CCC(C(C)(C)C)CC2)N([C@H](CCC(C)(C)C)c2ccc(C(=O)NCCC(=O)O)cc2)C1=O] |
| 10.11 | 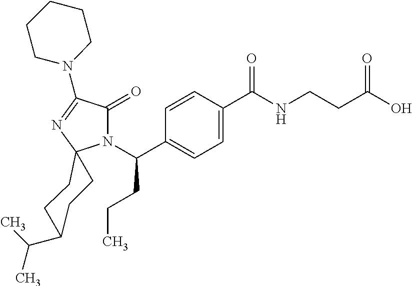 [SMILES: CCC[C@H](c1ccc(C(=O)NCCC(=O)O)cc1)N1C(=O)C(N2CCCCC2)=NC12CCC(C(C)C)CC2] |
| 10.22 | 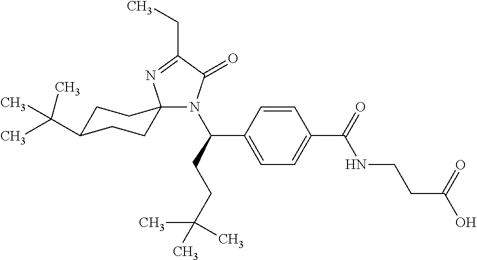 [SMILES: CCC1=NC2(CCC(C(C)(C)C)CC2)N([C@H](CCC(C)(C)C)c2ccc(C(=O)NCCC(=O)O)cc2)C1=O] |
| 10.23 | 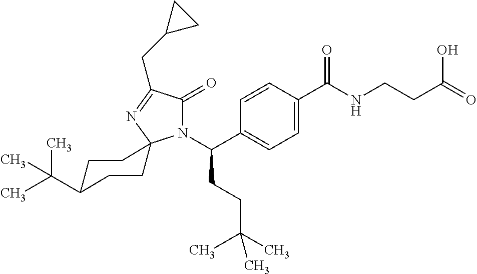 [SMILES: CC(C)(C)CC[C@H](c1ccc(C(=O)NCCC(=O)O)cc1)N1C(=O)C(CC2CC2)=NC12CCC(C(C)(C)C)CC2] |
| 10.24 | 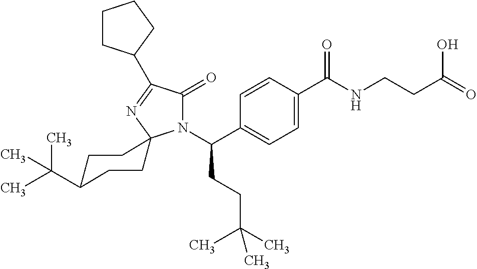 [SMILES: CC(C)(C)CC[C@H](c1ccc(C(=O)NCCC(=O)O)cc1)N1C(=O)C(C2CCCC2)=NC12CCC(C(C)(C)C)CC2] |
| 10.25 | 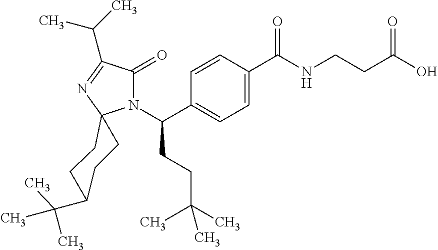 [SMILES: CC(C)C1=NC2(CCC(C(C)(C)C)CC2)N([C@H](CCC(C)(C)C)c2ccc(C(=O)NCCC(=O)O)cc2)C1=O] |
| 10.26 | 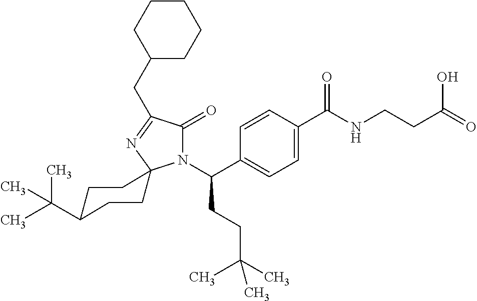 [SMILES: CC(C)(C)CC[C@H](c1ccc(C(=O)NCCC(=O)O)cc1)N1C(=O)C(CC2CCCCC2)=NC12CCC(C(C)(C)C)CC2] |
| 10.27 | 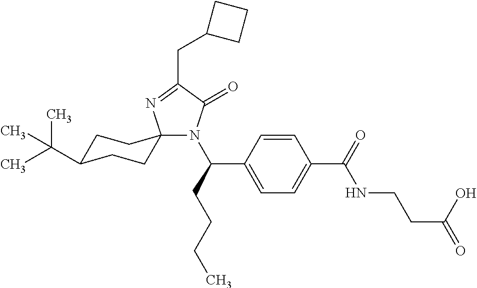 [SMILES: CCCC[C@H](c1ccc(C(=O)NCCC(=O)O)cc1)N1C(=O)C(CC2CCC2)=NC12CCC(C(C)(C)C)CC2] |
| 10.28 | 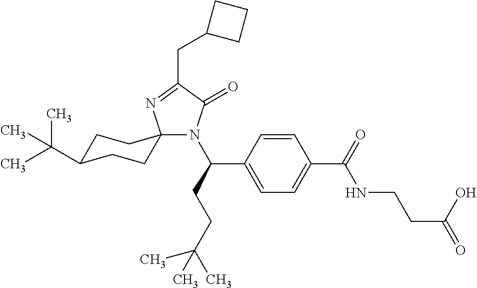 [SMILES: CC(C)(C)CC[C@H](c1ccc(C(=O)NCCC(=O)O)cc1)N1C(=O)C(CC2CCC2)=NC12CCC(C(C)(C)C)CC2] |
| 10.29 | 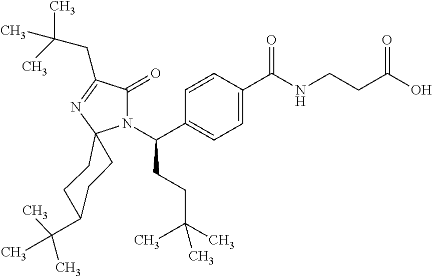 [SMILES: CC(C)(C)CC[C@H](c1ccc(C(=O)NCCC(=O)O)cc1)N1C(=O)C(CC(C)(C)C)=NC12CCC(C(C)(C)C)CC2] |
| 10.42 | 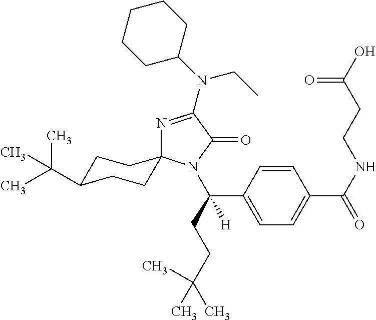 [SMILES: CCN(C1=NC2(CCC(C(C)(C)C)CC2)N([C@H](CCC(C)(C)C)c2ccc(C(=O)NCCC(=O)O)cc2)C1=O)C1CCCCC1] |
| 10.46 | 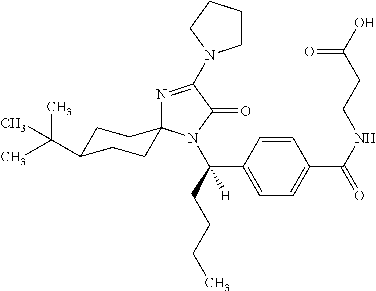 [SMILES: CCCC[C@H](c1ccc(C(=O)NCCC(=O)O)cc1)N1C(=O)C(N2CCCC2)=NC12CCC(C(C)(C)C)CC2] |
| 10.48 | 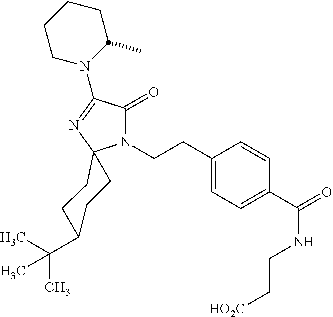 |
| 10.49 | 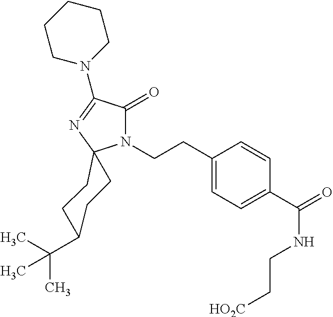 [SMILES: CC(C)(C)C1CCC2(CC1)N=C(N1CCCCC1)C(=O)N2CCc1ccc(C(=O)NCCC(=O)O)cc1] |

16. A compound of claim 1, or a pharmaceutically acceptable salt or tautomer of said compound, said compound having the structure:

| Ex. | Structure |
|---|---|
| 9.3 | 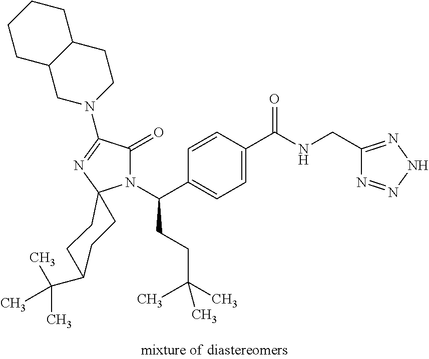 [SMILES: CC(C)(C)CC[C@H](c1ccc(C(=O)NCc2nn[nH]n2)cc1)N1C(=O)C(N2CCC3CCCCC3C2)=NC12CCC(C(C)(C)C)CC2] |

17. A composition comprising a compound according to claim 1 and a pharmaceutically acceptable carrier.

18. A composition of claim 17, further comprising one or more other antidiabetic agents.

19. (canceled)

20. A composition of claim 17, further comprising at least one additional therapeutic agent selected from the group consisting of: DPP-IV inhibitor, an insulin sensitizer, insulin, an insulin mimetic, an insulin secretagogue, a GLP-1 mimetic, a glucosidase inhibitor, an alpha glucosidase inhibitor, a glucagon receptor antagonist other than a compound of claim 1, glucophage, glucophage XR, an antihypertensive agent, a meglitinide, an alpha-glucosidase inhibitor, amlintide, pramlintide, exendin, a histamine H₃ receptor antagonist, dapagliflozin, sergliflozin, AVE2268 (Sanofi-Aventis) and T-1095 (Tanabe Seiyaku), a cholesterol lowering agent, a PACAP, a PACAP mimetic, a PACAP receptor 3 agonist, a PPAR delta agonist, an antiobesity agent, an ileal bile acid transporter inhibitor, an NSAID, and a CB1 receptor antagonist, and a CB1 receptor inverse agonist.

21. A method for treating type 2 diabetes mellitus in a patient in need thereof, comprising administering to said patient at least one compound according to claim 1 in an amount that is effective to treat type 2 diabetes mellitus.

22-31. (canceled)
